SEQUENCE LISTING (425 sequences)

SEQ ID NO: 1 - DNA, 125 bp, Synthetic construct
  source 1..125: mol_type = other DNA; note = source: AAV1; Recogn. Site: Nb.BbvCI; Format: bl
ttgcccactccccctcagcgcgctcgctcgctcggtggggcctgcggaccaaaggtccgcagacggcagagctctgctctgccggccccaccgagcgagcgagcgcgctgagggggagtgggcaa

SEQ ID NO: 2 - DNA, 125 bp, Synthetic construct
  source 1..125: mol_type = other DNA; note = source: AAV1; Recogn. Site: Nb.BbvCI; Format: tl
ttgcccactcccgctgagggcgctcgctcgctcggtggggcctgcggaccaaaggtccgcagacggcagagctctgctctgccggccccaccgagcgagcgagcgccctcagcgggagtgggcaa

SEQ ID NO: 3 - DNA, 85 bp, Synthetic construct
  source 1..85: mol_type = other DNA
gctcgactcgctcactgaggccgggcgaccaaaggtcgcccgacgcccgggctttgcccgggcggcctcagtgagcgagtcgagc

SEQ ID NO: 4 - DNA, 77 bp, Synthetic construct
  source 1..77: mol_type = other DNA
gctcgactcactgaggccgggcgaccaaaggtcgcccgacgcccgggctttgcccgggcggcctcagtgagtcgagc

SEQ ID NO: 5 - DNA, 69 bp, Synthetic construct
  source 1..69: mol_type = other DNA
cgctgactcaggccgggcgaccaaaggtcgcccgacgcccgggctttgcccgggcggcctgagtcagcg

SEQ ID NO: 6 - skipped

SEQ ID NO: 7 - DNA, 89 bp, Synthetic construct
  source 1..89: mol_type = other DNA
cgcgctcgctcgctcactgaggccgggcgaccaaaggtcgcccgacgcccgggctttgcccgggcggcctcagtgagcgagcgagcgcg

SEQ ID NO: 8 - DNA, 71 bp, Synthetic construct
  source 1..71: mol_type = other DNA
tcgctcactgaggccgggcgaccaaaggtcgcccgacgcccgggctttgcccgggcggcctcagtgagcga

SEQ ID NO: 9 - DNA, 59 bp, Synthetic construct
  source 1..59: mol_type = other DNA
actgaggccgggcgaccaaaggtcgcccgacgcccgggctttgcccgggcggcctcagt

SEQ ID NO: 10 - DNA, 51 bp, Synthetic construct
  source 1..51: mol_type = other DNA
aggccgggcgaccaaaggtcgcccgacgcccgggctttgcccgggcggcct

SEQ ID NO: 11 - DNA, 125 bp, Synthetic construct
  source 1..125: mol_type = other DNA; note = AAV1 Left ITR
ttgcccactccctctctgcgcgctcgctcgctcggtggggcctgcggaccaaaggtccgcagacggcagagctctgctctgccggccccaccgagcgagcgagcgcgcagagagggagtgggcaa

SEQ ID NO: 12 - DNA, 125 bp, Synthetic construct; identical to SEQ ID NO: 11
  source 1..125: mol_type = other DNA; note = AAV1 Right ITR

SEQ ID NO: 13 - DNA, 125 bp, Synthetic construct
  source 1..125: mol_type = other DNA; note = AAV2 Left ITR
ttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 14 - DNA, 125 bp, Synthetic construct; identical to SEQ ID NO: 13
  source 1..125: mol_type = other DNA; note = AAV2 Right ITR

SEQ ID NO: 15 - DNA, 125 bp, Synthetic construct
  source 1..125: mol_type = other DNA; note = AAV3 Left ITR
ttggccactccctctatgcgcactcgctcgctcggtggggcctggcgaccaaaggtcgccagacggacgtgctttgcacgtccggccccaccgagcgagcgagtgcgcatagagggagtggccaa

SEQ ID NO: 16 - DNA, 125 bp, Synthetic construct; identical to SEQ ID NO: 15
  source 1..125: mol_type = other DNA; note = AAV3 Right ITR

SEQ ID NO: 17 - DNA, 125 bp, Synthetic construct
  source 1..125: mol_type = other DNA; note = AAV4 Left ITR
ttggccactccctctatgcgcgctcgctcactcactcggccctggagaccaaaggtctccagactgccggcctctggccggcagggccgagtgagtgagcgagcgcgcatagagggagtggccaa

SEQ ID NO: 18 - DNA, 112 bp, Synthetic construct
  source 1..112: mol_type = other DNA; note = AAV4 Right ITR
ctatgcgcgctcgctcactcactcggccctggagaccaaaggtctccagactgccggcctctggccggcagggccgagtgagtgagcgagcgcgcatagagggagtggccaa

SEQ ID NO: 19 - DNA, 137 bp, Synthetic construct
  source 1..137: mol_type = other DNA; note = AAV5 (NC_006152) Left ITR
ctctcccccctgtcgcgttcgctcgctcgctggctcgtttgggggggtggcagctcaaagagctgccagacgacggccctctggccgtcgcccccccaaacgagccagcgagcgagcgaacgcgacaggggggagag

SEQ ID NO: 20 - DNA, 137 bp, Synthetic construct; identical to SEQ ID NO: 19
  source 1..137: mol_type = other DNA; note = AAV5 (NC_006152) Right ITR

SEQ ID NO: 21 - DNA, 125 bp, Synthetic construct
  source 1..125: mol_type = other DNA; note = AAV7 (NC_006260) Left ITR
ttggccactccctctatgcgcgctcgctcgctcggtggggcctgcggaccaaaggtccgcagacggcagagctctgctctgccggccccaccgagcgagcgagcgcgcatagagggagtggccaa

SEQ ID NO: 22 - DNA, 125 bp, Synthetic construct; identical to SEQ ID NO: 21
  source 1..125: mol_type = other DNA; note = AAV7 (NC_006260) Right ITR

SEQ ID NO: 23 - DNA, 140 bp, Synthetic construct
  source 1..140: mol_type = other DNA; note = HBOV (JQ923422) Left ITR
gtggttgtacagacgccatcttggaatccaatatgtctgccggctcagtcatgcctgcgctgcgcgcagcgcgctgcgcgcgcgcatgatctaatcgccggcagacatattggattccaagatggcgtctgtacaaccac

SEQ ID NO: 24 - DNA, 200 bp, Synthetic construct
  source 1..200: mol_type = other DNA; note = HBOV (JQ923422) Right ITR
ttgcttatgcaatcgcgaaactctatatcttttaatgtgttgttgttgtacatgcgccatcttagttttatatcagctggcgccttagttatataacatgcatgttatataactaaggcgccagctgatataaaactaagatggcgcatgtacaacaacaacacattaaaagatatagagtttcgcgattgcataagcaa

SEQ ID NO: 25 - DNA, 89 bp, Synthetic construct
  source 1..89: mol_type = other DNA; note = hB19 (AY386330) Left ITR
tgggccagcttgcttggggttgccttgacactaagacaagcggcgcgccgcttgatcttagtggcacgtcaaccccaagcgctggccca

SEQ ID NO: 26 - DNA, 89 bp, Synthetic construct
  source 1..89: mol_type = other DNA; note = hB19 (AY386330) Right ITR
tgggccagcgcttggggttgacgtgccactaagatcaagcggcgcgccgcttgtcttagtgtcaaggcaaccccaagcaagctggccca

SEQ ID NO: 27 - DNA, 70 bp, Synthetic construct
  source 1..70: mol_type = other DNA
ccatgcatccggctttaaacgggcaactgcgtctcattcacgttagagactacaaccgtcggatgcatgg

SEQ ID NO: 28 - DNA, 102 bp, Synthetic construct
  source 1..102: mol_type = other DNA
ttcaaacctgccgggggagaagcggcgttttttcccggccgccgcttctcttcttctcccgccgccgggaaaaaaggcgggagaagccccggcaggtttgaa

SEQ ID NO: 29 - DNA, 92 bp, Synthetic construct
  source 1..92: mol_type = other DNA
gtccgggccatgcttcaaacctgccggggcttctcccgccttttttcccggcggcgggagaagtagatttctcgtacctgcatggcccggac

SEQ ID NO: 30 - DNA, 50 bp, Synthetic construct
  source 1..50: mol_type = other DNA; note = OriL hairpin sequence
gaagagggcggcggcccttttttccgccctcttcggggccgtccaaactt

SEQ ID NO: 31 - DNA, 87 bp, Synthetic construct
  source 1..87: mol_type = other DNA
ccagcgcttggggttgacgtgccactaagatcaagcggcgcgcgcgcgccgcttgtcttagtgtcaaggcaaccccaagcaagctgg

SEQ ID NO: 32 - DNA, 113 bp, Synthetic construct
  source 1..113: mol_type = other DNA
ggttgactctgggccagcttgcttggggttgccttgacactaagacaagcggcgcgcgcgcgccgcttgatcttagtggcacgtcaaccccaagcgctggcccagagtcaacc

SEQ ID NO: 33 - DNA, 83 bp, Synthetic construct
  source 1..83: mol_type = other DNA
cgcgctcgctcgctcactgaggccgggccaaaggcccgacgcccgggctttgcccgggcggcctcagtgagcgagcgagcgcg

SEQ ID NO: 34 - DNA, 83 bp, Synthetic construct
  source 1..83: mol_type = other DNA
cgcgctcgctcgctcactgaggccgggcgaccaaaggtcgcccgacgcccgtttcgggcggcctcagtgagcgagcgagcgcg

SEQ ID NO: 35 - DNA, 67 bp, Synthetic construct
  source 1..67: mol_type = other DNA
cgcgctcgctcgctcactgaggccgcccgggctttgcccgggcggcctcagtgagcgagcgagcgcg

SEQ ID NO: 36 - DNA, 125 bp, Synthetic construct
  source 1..125: mol_type = other DNA; note = source: AAV2; Recogn. Site: Nb.BbvCI; Format: bl
ttggccactccccctcagcgcgctcgctcgctcactgaggccgggcgaccaaaggtcgcccgacgcccgggctttgcccgggcggcctcagtgagcgagcgagcgcgctgagggggagtggccaa

SEQ ID NO: 37 - DNA, 125 bp, Synthetic construct
  source 1..125: mol_type = other DNA; note = source: AAV2; Recogn. Site: Nb.BbvCI; Format: tl
ttggccactcccgctgagggcgctcgctcgctcactgaggccgggcgaccaaaggtcgcccgacgcccgggctttgcccgggcggcctcagtgagcgagcgagcgccctcagcgggagtggccaa

SEQ ID NO: 38 - DNA, 125 bp, Synthetic construct
  source 1..125: mol_type = other DNA; note = source: AAV3; Recogn. Site: Nb.BbvCI; Format: bl
ttggccactccccctcagcgcactcgctcgctcggtggggcctggcgaccaaaggtcgccagacggacgtgctttgcacgtccggccccaccgagcgagcgagtgcgctgagggggagtggccaa

SEQ ID NO: 39 - DNA, 125 bp, Synthetic construct
  source 1..125: mol_type = other DNA; note = source: AAV3; Recogn. Site: Nb.BbvCI; Format: tl
ttggccactcccgctgagggcactcgctcgctcggtggggcctggcgaccaaaggtcgccagacggacgtgctttgcacgtccggccccaccgagcgagcgagtgccctcagcgggagtggccaa

SEQ ID NO: 40 - DNA, 125 bp, Synthetic construct
  source 1..125: mol_type = other DNA; note = source: AAV4 left; Recogn. Site: Nb.BbvCI; Format: bl
ttggccactccccctcagcgcgctcgctcactcactcggccctggagaccaaaggtctccagactgccggcctctggccggcagggccgagtgagtgagcgagcgcgctgagggggagtggccaa

SEQ ID NO: 41 - DNA, 125 bp, Synthetic construct
  source 1..125: mol_type = other DNA; note = source: AAV4 left; Recogn. Site: Nb.BbvCI; Format: tl
ttggccactcccgctgagggcgctcgctcactcactcggccctggagaccaaaggtctccagactgccggcctctggccggcagggccgagtgagtgagcgagcgccctcagcgggagtggccaa

SEQ ID NO: 42 - DNA, 125 bp, Synthetic construct
  source 1..125: mol_type = other DNA; note = source: AAV4 right; Recogn. Site: Nb.BbvCI; Format: bl
ttggccacattacctcagcgcgctcgctcactcactcggccctggagaccaaaggtctccagactgccggcctctggccggcagggccgagtgagtgagcgagcgcgctgagggggagtggccaa

SEQ ID NO: 43 - DNA, 125 bp, Synthetic construct
  source 1..125: mol_type = other DNA; note = source: AAV4 right; Recogn. Site: Nb.BbvCI; Format: tl
ttggccacattagctgagggcgctcgctcactcactcggccctggagaccaaaggtctccagactgccggcctctggccggcagggccgagtgagtgagcgagcgccctcagcgggagtggccaa

SEQ ID NO: 44 - DNA, 137 bp, Synthetic construct
  source 1..137: mol_type = other DNA; note = source: AAV5; Recogn. Site: Nb.BbvCI; Format: bl
ctctcccctcagccgcgttcgctcgctcgctggctcgtttgggggggtggcagctcaaagagctgccagacgacggccctctggccgtcgcccccccaaacgagccagcgagcgagcgaacgcggctgaggggagag

SEQ ID NO: 45 - DNA, 137 bp, Synthetic construct
  source 1..137: mol_type = other DNA; note = source: AAV5; Recogn. Site: Nb.BbvCI; Format: tl
ctctccccgctgaggcgttcgctcgctcgctggctcgtttgggggggtggcagctcaaagagctgccagacgacggccctctggccgtcgcccccccaaacgagccagcgagcgagcgaacgcctcagcggggagag

SEQ ID NO: 46 - DNA, 125 bp, Synthetic construct
  source 1..125: mol_type = other DNA; note = source: AAV7; Recogn. Site: Nb.BbvCI; Format: bl
ttggccactccccctcagcgcgctcgctcgctcggtggggcctgcggaccaaaggtccgcagacggcagagctctgctctgccggccccaccgagcgagcgagcgcgctgagggggagtggccaa

SEQ ID NO: 47 - DNA, 125 bp, Synthetic construct
  source 1..125: mol_type = other DNA; note = source: AAV7; Recogn. Site: Nb.BbvCI; Format: tl
ttggccactcccgctgagggcgctcgctcgctcggtggggcctgcggaccaaaggtccgcagacggcagagctctgctctgccggccccaccgagcgagcgagcgccctcagcgggagtggccaa

SEQ ID NO: 48 - DNA, 125 bp, Synthetic construct
  source 1..125: mol_type = other DNA; note = source: AAV1; Recogn. Site: Nb.BsmI; Format: bl
ttgcccactccctgaatgcgcgctcgctcgctcggtggggcctgcggaccaaaggtccgcagacggcagagctctgctctgccggccccaccgagcgagcgagcgcgcattcagggagtgggcaa

SEQ ID NO: 49 - DNA, 125 bp, Synthetic construct
  source 1..125: mol_type = other DNA; note = source: AAV1; Recogn. Site: Nb.BsmI; Format: tl
ttgcccactccctctctgcgcattcgctcgctcggtggggcctgcggaccaaaggtccgcagacggcagagctctgctctgccggccccaccgagcgagcgaatgcgcagagagggagtgggcaa

SEQ ID NO: 50 - DNA, 125 bp, Synthetic construct
  source 1..125: mol_type = other DNA; note = source: AAV2; Recogn. Site: Nb.BsmI; Format: bl
ttggccactccctgaatgcgcgctcgctcgctcactgaggccgggcgaccaaaggtcgcccgacgcccgggctttgcccgggcggcctcagtgagcgagcgagcgcgcattcagggagtggccaa

SEQ ID NO: 51 - DNA, 125 bp, Synthetic construct
  source 1..125: mol_type = other DNA; note = source: AAV2; Recogn. Site: Nb.BsmI; Format: tl
ttggccactccctctctgcgcattcgctcgctcactgaggccgggcgaccaaaggtcgcccgacgcccgggctttgcccgggcggcctcagtgagcgagcgaatgcgcagagagggagtggccaa

SEQ ID NO: 52 - DNA, 125 bp, Synthetic construct
  source 1..125: mol_type = other DNA; note = source: AAV3; Recogn. Site: Nb.BsmI; Format: bl
ttggccactccctgaatgcgcactcgctcgctcggtggggcctggcgaccaaaggtcgccagacggacgtgctttgcacgtccggccccaccgagcgagcgagtgcgcattcagggagtggccaa

SEQ ID NO: 53 - DNA, 125 bp, Synthetic construct
  source 1..125: mol_type = other DNA; note = source: AAV3; Recogn. Site: Nb.BsmI; Format: tl
ttggccactccctctatgcgcattcgctcgctcggtggggcctggcgaccaaaggtcgccagacggacgtgctttgcacgtccggccccaccgagcgagcgaatgcgcatagagggagtggccaa

SEQ ID NO: 54 - DNA, 125 bp, Synthetic construct
  source 1..125: mol_type = other DNA; note = source: AAV4 left; Recogn. Site: Nb.BsmI; Format: bl
ttggccactccctgaatgcgcgctcgctcactcactcggccctggagaccaaaggtctccagactgccggcctctggccggcagggccgagtgagtgagcgagcgcgcattcagggagtggccaa

SEQ ID NO: 55 - DNA, 125 bp, Synthetic construct
  source 1..125: mol_type = other DNA; note = source: AAV4 left; Recogn. Site: Nb.BsmI; Format: tl
ttggccactccctctatgcgcattcgctcactcactcggccctggagaccaaaggtctccagactgccggcctctggccggcagggccgagtgagtgagcgaatgcgcatagagggagtggccaa

SEQ ID NO: 56 - DNA, 125 bp, Synthetic construct
  source 1..125: mol_type = other DNA; note = source: AAV4 right; Recogn. Site: Nb.BsmI; Format: bl
ttggccacattaggaatgcgcgctcgctcactcactcggccctggagaccaaaggtctccagactgccggcctctggccggcagggccgagtgagtgagcgagcgcgcattcagggagtggccaa

SEQ ID NO: 57 - DNA, 125 bp, Synthetic construct
  source 1..125: mol_type = other DNA; note = source: AAV4 right; Recogn. Site: Nb.BsmI; Format: tl
ttggccacattagctatgcgcattcgctcactcactcggccctggagaccaaaggtctccagactgccggcctctggccggcagggccgagtgagtgagcgaatgcgcatagagggagtggccaa

SEQ ID NO: 58 - DNA, 137 bp, Synthetic construct
  source 1..137: mol_type = other DNA; note = source: AAV5; Recogn. Site: Nb.BsmI; Format: bl
ctctccccgaatgcgcgttcgctcgctcgctggctcgtttgggggggtggcagctcaaagagctgccagacgacggccctctggccgtcgcccccccaaacgagccagcgagcgagcgaacgcgcattcggggagag

SEQ ID NO: 59 - DNA, 137 bp, Synthetic construct
  source 1..137: mol_type = other DNA; note = source: AAV5; Recogn. Site: Nb.BsmI; Format: tl
ctctcccccctgtcgcattcgctcgctcgctggctcgtttgggggggtggcagctcaaagagctgccagacgacggccctctggccgtcgcccccccaaacgagccagcgagcgagcgaatgcgacaggggggagag

SEQ ID NO: 60 - DNA, 125 bp, Synthetic construct
  source 1..125: mol_type = other DNA; note = source: AAV7; Recogn. Site: Nb.BsmI; Format: bl
ttggccactccctgaatgcgcgctcgctcgctcggtggggcctgcggaccaaaggtccgcagacggcagagctctgctctgccggccccaccgagcgagcgagcgcgcattcagggagtggccaa

SEQ ID NO: 61 - DNA, 125 bp, Synthetic construct
  source 1..125: mol_type = other DNA; note = source: AAV7; Recogn. Site: Nb.BsmI; Format: tl
ttggccactccctctatgcgcattcgctcgctcggtggggcctgcggaccaaaggtccgcagacggcagagctctgctctgccggccccaccgagcgagcgaatgcgcatagagggagtggccaa

SEQ ID NO: 62 - DNA, 125 bp, Synthetic construct
  source 1..125: mol_type = other DNA; note = source: AAV1; Recogn. Site: Nb.BsrDI; Format: bl
ttgcccactcccgcaatgcgcgctcgctcgctcggtggggcctgcggaccaaaggtccgcagacggcagagctctgctctgccggccccaccgagcgagcgagcgcgcattgcgggagtgggcaa

SEQ ID NO: 63 - DNA, 125 bp, Synthetic construct
  source 1..125: mol_type = other DNA; note = source: AAV1; Recogn. Site: Nb.BsrDI; Format: tl
ttgcccactccctcattgcgcgctcgctcgctcggtggggcctgcggaccaaaggtccgcagacggcagagctctgctctgccggccccaccgagcgagcgagcgcgcaatgagggagtgggcaa

SEQ ID NO: 64 - DNA, 125 bp, Synthetic construct
  source 1..125: mol_type = other DNA; note = source: AAV2; Recogn. Site: Nb.BsrDI; Format: bl
ttggccactcccgcaatgcgcgctcgctcgctcactgaggccgggcgaccaaaggtcgcccgacgcccgggctttgcccgggcggcctcagtgagcgagcgagcgcgcattgcgggagtggccaa

SEQ ID NO: 65 - DNA, 125 bp, Synthetic construct
  source 1..125: mol_type = other DNA; note = source: AAV2; Recogn. Site: Nb.BsrDI; Format: tl
ttggccactccctcattgcgcgctcgctcgctcactgaggccgggcgaccaaaggtcgcccgacgcccgggctttgcccgggcggcctcagtgagcgagcgagcgcgcaatgagggagtggccaa

SEQ ID NO: 66 - DNA, 125 bp, Synthetic construct
  source 1..125: mol_type = other DNA; note = source: AAV3; Recogn. Site: Nb.BsrDI; Format: bl
ttggccactcccgcaatgcgcactcgctcgctcggtggggcctggcgaccaaaggtcgccagacggacgtgctttgcacgtccggccccaccgagcgagcgagtgcgcattgcgggagtggccaa

SEQ ID NO: 67 - DNA, 125 bp, Synthetic construct
  source 1..125: mol_type = other DNA; note = source: AAV3; Recogn. Site: Nb.BsrDI; Format: tl
ttggccactccctcattgcgcactcgctcgctcggtggggcctggcgaccaaaggtcgccagacggacgtgctttgcacgtccggccccaccgagcgagcgagtgcgcaatgagggagtggccaa

SEQ ID NO: 68 - DNA, 125 bp, Synthetic construct
  source 1..125: mol_type = other DNA; note = source: AAV4 left; Recogn. Site: Nb.BsrDI; Format: bl
ttggccactcccgcaatgcgcgctcgctcactcactcggccctggagaccaaaggtctccagactgccggcctctggccggcagggccgagtgagtgagcgagcgcgcattgcgggagtggccaa

SEQ ID NO: 69 - DNA, 125 bp, Synthetic construct
  source 1..125: mol_type = other DNA; note = source: AAV4 left; Recogn. Site: Nb.BsrDI; Format: tl
ttggccactccctcattgcgcgctcgctcactcactcggccctggagaccaaaggtctccagactgccggcctctggccggcagggccgagtgagtgagcgagcgcgcaatgagggagtggccaa

SEQ ID NO: 70 - DNA, 125 bp, Synthetic construct
  source 1..125: mol_type = other DNA; note = source: AAV4 right; Recogn. Site: Nb.BsrDI; Format: bl
ttggccacattagcaatgcgcgctcgctcactcactcggccctggagaccaaaggtctccagactgccggcctctggccggcagggccgagtgagtgagcgagcgcgcattgcgggagtggccaa

SEQ ID NO: 71 - DNA, 125 bp, Synthetic construct
  source 1..125: mol_type = other DNA; note = source: AAV4 right; Recogn. Site: Nb.BsrDI; Format: tl
ttggccacattagcattgcgcgctcgctcactcactcggccctggagaccaaaggtctccagactgccggcctctggccggcagggccgagtgagtgagcgagcgcgcaatgagggagtggccaa

SEQ ID NO: 72 - DNA, 137 bp, Synthetic construct
  source 1..137: mol_type = other DNA; note = source: AAV5; Recogn. Site: Nb.BsrDI; Format: bl
ctctccgcaatgtcgcgttcgctcgctcgctggctcgtttgggggggtggcagctcaaagagctgccagacgacggccctctggccgtcgcccccccaaacgagccagcgagcgagcgaacgcgacattgcggagag

SEQ ID NO: 73 - DNA, 137 bp, Synthetic construct
  source 1..137: mol_type = other DNA; note = source: AAV5; Recogn. Site: Nb.BsrDI; Format: tl
ctctcccccattgcgcgttcgctcgctcgctggctcgtttgggggggtggcagctcaaagagctgccagacgacggccctctggccgtcgcccccccaaacgagccagcgagcgagcgaacgcgcaatgggggagag

SEQ ID NO: 74 - DNA, 125 bp, Synthetic construct
  source 1..125: mol_type = other DNA; note = source: AAV7; Recogn. Site: Nb.BsrDI; Format: bl
ttggccactcccgcaatgcgcgctcgctcgctcggtggggcctgcggaccaaaggtccgcagacggcagagctctgctctgccggccccaccgagcgagcgagcgcgcattgcgggagtggccaa

SEQ ID NO: 75 - DNA, 125 bp, Synthetic construct
  source 1..125: mol_type = other DNA; note = source: AAV7; Recogn. Site: Nb.BsrDI; Format: tl
ttggccactccctcattgcgcgctcgctcgctcggtggggcctgcggaccaaaggtccgcagacggcagagctctgctctgccggccccaccgagcgagcgagcgcgcaatgagggagtggccaa

SEQ ID NO: 76 - DNA, 125 bp, Synthetic construct
  source 1..125: mol_type = other DNA; note = source: AAV1; Recogn. Site: Nb.BssSI; Format: bl
ttgcccacgagctctctgcgcgctcgctcgctcggtggggcctgcggaccaaaggtccgcagacggcagagctctgctctgccggccccaccgagcgagcgagcgcgcagagagctcgtgggcaa

SEQ ID NO: 77 - DNA, 125 bp, Synthetic construct
  source 1..125: mol_type = other DNA; note = source: AAV1; Recogn. Site: Nb.BssSI; Format: tl
ttgcccactccctcgtggcgcgctcgctcgctcggtggggcctgcggaccaaaggtccgcagacggcagagctctgctctgccggccccaccgagcgagcgagcgcgccacgagggagtgggcaa

SEQ ID NO: 78 - DNA, 125 bp, Synthetic construct
  source 1..125: mol_type = other DNA; note = source: AAV2; Recogn. Site: Nb.BssSI; Format: bl
ttggccacgagctctctgcgcgctcgctcgctcactgaggccgggcgaccaaaggtcgcccgacgcccgggctttgcccgggcggcctcagtgagcgagcgagcgcgcagagagctcgtggccaa

SEQ ID NO: 79 - DNA, 125 bp, Synthetic construct
  source 1..125: mol_type = other DNA; note = source: AAV2; Recogn. Site: Nb.BssSI; Format: tl
ttggccactccctcgtggcgcgctcgctcgctcactgaggccgggcgaccaaaggtcgcccgacgcccgggctttgcccgggcggcctcagtgagcgagcgagcgcgccacgagggagtggccaa

SEQ ID NO: 80 - DNA, 125 bp, Synthetic construct
  source 1..125: mol_type = other DNA; note = source: AAV3; Recogn. Site: Nb.BssSI; Format: bl
ttggccacgagctctatgcgcactcgctcgctcggtggggcctggcgaccaaaggtcgccagacggacgtgctttgcacgtccggccccaccgagcgagcgagtgcgcatagagctcgtggccaa

SEQ ID NO: 81 - DNA, 125 bp, Synthetic construct
  source 1..125: mol_type = other DNA; note = source: AAV3; Recogn. Site: Nb.BssSI; Format: tl
ttggccactccctcgtggcgcactcgctcgctcggtggggcctggcgaccaaaggtcgccagacggacgtgctttgcacgtccggccccaccgagcgagcgagtgcgccacgagggagtggccaa

SEQ ID NO: 82 - DNA, 125 bp, Synthetic construct
  source 1..125: mol_type = other DNA; note = source: AAV4 left; Recogn. Site: Nb.BssSI; Format: bl
ttggccacgagctctatgcgcgctcgctcactcactcggccctggagaccaaaggtctccagactgccggcctctggccggcagggccgagtgagtgagcgagcgcgcatagagctcgtggccaa

SEQ ID NO: 83 - DNA, 125 bp, Synthetic construct
  source 1..125: mol_type = other DNA; note = source: AAV4 left; Recogn. Site: Nb.BssSI; Format: tl
ttggccactccctcgtggcgcgctcgctcactcactcggccctggagaccaaaggtctccagactgccggcctctggccggcagggccgagtgagtgagcgagcgcgccacgagggagtggccaa

SEQ ID NO: 84 - DNA, 125 bp, Synthetic construct
  source 1..125: mol_type = other DNA; note = source: AAV4 right; Recogn. Site: Nb.BssSI; Format: bl
ttggccacgagagctatgcgcgctcgctcactcactcggccctggagaccaaaggtctccagactgccggcctctggccggcagggccgagtgagtgagcgagcgcgcatagagctcgtggccaa

SEQ ID NO: 85 - DNA, 125 bp, Synthetic construct
  source 1..125: mol_type = other DNA; note = source: AAV4 right; Recogn. Site: Nb.BssSI; Format: tl
ttggccacattctcgtggcgcgctcgctcactcactcggccctggagaccaaaggtctccagactgccggcctctggccggcagggccgagtgagtgagcgagcgcgccacgagggagtggccaa

SEQ ID NO: 86 - DNA, 137 bp, Synthetic construct
  source 1..137: mol_type = other DNA; note = source: AAV5; Recogn. Site: Nb.BssSI; Format: bl
ctcacgagcctgtcgcgttcgctcgctcgctggctcgtttgggggggtggcagctcaaagagctgccagacgacggccctctggccgtcgcccccccaaacgagccagcgagcgagcgaacgcgacaggctcgtgag

SEQ ID NO: 87 - DNA, 137 bp, Synthetic construct
  source 1..137: mol_type = other DNA; note = source: AAV5; Recogn. Site: Nb.BssSI; Format: tl
ctctccctcgtgtcgcgttcgctcgctcgctggctcgtttgggggggtggcagctcaaagagctgccagacgacggccctctggccgtcgcccccccaaacgagccagcgagcgagcgaacgcgacacgagggagag

SEQ ID NO: 88 - DNA, 125 bp, Synthetic construct
  source 1..125: mol_type = other DNA; note = source: AAV7; Recogn. Site: Nb.BssSI; Format: bl
ttggccacgagctctatgcgcgctcgctcgctcggtggggcctgcggaccaaaggtccgcagacggcagagctctgctctgccggccccaccgagcgagcgagcgcgcatagagctcgtggccaa

SEQ ID NO: 89 - DNA, 125 bp, Synthetic construct
  source 1..125: mol_type = other DNA; note = source: AAV7; Recogn. Site: Nb.BssSI; Format: tl
ttggccactccctcgtggcgcgctcgctcgctcggtggggcctgcggaccaaaggtccgcagacggcagagctctgctctgccggccccaccgagcgagcgagcgcgccacgagggagtggccaa

SEQ ID NO: 90 - DNA, 125 bp, Synthetic construct
  source 1..125: mol_type = other DNA; note = source: AAV1; Recogn. Site: Nb.BtsI; Format: bl
ttgcccactcccgcagtgcgcgctcgctcgctcggtggggcctgcggaccaaaggtccgcagacggcagagctctgctctgccggccccaccgagcgagcgagcgcgcactgcgggagtgggcaa

SEQ ID NO: 91 - DNA, 125 bp, Synthetic construct
  source 1..125: mol_type = other DNA; note = source: AAV1; Recogn. Site: Nb.BtsI; Format: tl
ttgcccactccctcactgcgcgctcgctcgctcggtggggcctgcggaccaaaggtccgcagacggcagagctctgctctgccggccccaccgagcgagcgagcgcgcagtgagggagtgggcaa

SEQ ID NO: 92 - DNA, 125 bp, Synthetic construct
  source 1..125: mol_type = other DNA; note = source: AAV2; Recogn. Site: Nb.BtsI; Format: bl
ttggccactcccgcagtgcgcgctcgctcgctcactgaggccgggcgaccaaaggtcgcccgacgcccgggctttgcccgggcggcctcagtgagcgagcgagcgcgcactgcgggagtggccaa

SEQ ID NO: 93 - DNA, 125 bp, Synthetic construct
  source 1..125: mol_type = other DNA; note = source: AAV2; Recogn. Site: Nb.BtsI; Format: tl
ttggccactccctcactgcgcgctcgctcgctcactgaggccgggcgaccaaaggtcgcccgacgcccgggctttgcccgggcggcctcagtgagcgagcgagcgcgcagtgagggagtggccaa

SEQ ID NO: 94 - DNA, 125 bp, Synthetic construct
  source 1..125: mol_type = other DNA; note = source: AAV3; Recogn. Site: Nb.BtsI; Format: bl
ttggccactcccgcagtgcgcactcgctcgctcggtggggcctggcgaccaaaggtcgccagacggacgtgctttgcacgtccggccccaccgagcgagcgagtgcgcactgcgggagtggccaa

SEQ ID NO: 95 - DNA, 125 bp, Synthetic construct
  source 1..125: mol_type = other DNA; note = source: AAV3; Recogn. Site: Nb.BtsI; Format: tl
ttggccactccctcactgcgcactcgctcgctcggtggggcctggcgaccaaaggtcgccagacggacgtgctttgcacgtccggccccaccgagcgagcgagtgcgcagtgagggagtggccaa

SEQ ID NO: 96 - DNA, 125 bp, Synthetic construct
  source 1..125: mol_type = other DNA; note = source: AAV4 left; Recogn. Site: Nb.BtsI; Format: bl
ttggccactcccgcagtgcgcgctcgctcactcactcggccctggagaccaaaggtctccagactgccggcctctggccggcagggccgagtgagtgagcgagcgcgcactgcgggagtggccaa

SEQ ID NO: 97 - DNA, 125 bp, Synthetic construct
  source 1..125: mol_type = other DNA; note = source: AAV4 left; Recogn. Site: Nb.BtsI; Format: tl
ttggccactccctcactgcgcgctcgctcactcactcggccctggagaccaaaggtctccagactgccggcctctggccggcagggccgagtgagtgagcgagcgcgcagtgagggagtggccaa

SEQ ID NO: 98 - DNA, 125 bp, Synthetic construct
  source 1..125: mol_type = other DNA; note = source: AAV4 right; Recogn. Site: Nb.BtsI; Format: bl
ttggccacattagcagtgcgcgctcgctcactcactcggccctggagaccaaaggtctccagactgccggcctctggccggcagggccgagtgagtgagcgagcgcgcactgcgggagtggccaa

SEQ ID NO: 99 - DNA, 125 bp, Synthetic construct
  source 1..125: mol_type = other DNA; note = source: AAV4 right; Recogn. Site: Nb.BtsI; Format: tl
ttggccacattagcactgcgcgctcgctcactcactcggccctggagaccaaaggtctccagactgccggcctctggccggcagggccgagtgagtgagcgagcgcgcagtgagggagtggccaa

SEQ ID NO: 100 - DNA, 137 bp, Synthetic construct
  source 1..137: mol_type = other DNA; note = source: AAV5; Recogn. Site: Nb.BtsI; Format: bl
ctctccgcagtgtcgcgttcgctcgctcgctggctcgtttgggggggtggcagctcaaagagctgccagacgacggccctctggccgtcgcccccccaaacgagccagcgagcgagcgaacgcgacactgcggagag

SEQ ID NO: 101 - DNA, 137 bp, Synthetic construct
  source 1..137: mol_type = other DNA; note = source: AAV5; Recogn. Site: Nb.BtsI; Format: tl
ctctccccactgccgcgttcgctcgctcgctggctcgtttgggggggtggcagctcaaagagctgccagacgacggccctctggccgtcgcccccccaaacgagccagcgagcgagcgaacgcggcagtggggagag

SEQ ID NO: 102 - DNA, 125 bp, Synthetic construct
  source 1..125: mol_type = other DNA; note = source: AAV7; Recogn. Site: Nb.BtsI; Format: bl
ttggccactcccgcagtgcgcgctcgctcgctcggtggggcctgcggaccaaaggtccgcagacggcagagctctgctctgccggccccaccgagcgagcgagcgcgcactgcgggagtggccaa

SEQ ID NO: 103 - DNA, 125 bp, Synthetic construct
  source 1..125: mol_type = other DNA; note = source: AAV7; Recogn. Site: Nb.BtsI; Format: tl
ttggccactccctcactgcgcgctcgctcgctcggtggggcctgcggaccaaaggtccgcagacggcagagctctgctctgccggccccaccgagcgagcgagcgcgcagtgagggagtggccaa

SEQ ID NO: 104 - DNA, 125 bp, Synthetic construct
  source 1..125: mol_type = other DNA; note = source: AAV1; Recogn. Site: Nt.AlwI; Format: bl
ttgcccactccctcgatccgcgctcgctcgctcggtggggcctgcggaccaaaggtccgcagacggcagagctctgctctgccggccccaccgagcgagcgagcgcggatcgagggagtgggcaa

SEQ ID NO: 105 - DNA, 125 bp, Synthetic construct
  source 1..125: mol_type = other DNA; note = source: AAV1; Recogn. Site: Nt.AlwI; Format: tl
ttgcccactggatctctgcgcgctcgctcgctcggtggggcctgcggaccaaaggtccgcagacggcagagctctgctctgccggccccaccgagcgagcgagcgcgcagagatccagtgggcaa

SEQ ID NO: 106 - DNA, 125 bp, Synthetic construct
  source 1..125: mol_type = other DNA; note = source: AAV2; Recogn. Site: Nt.AlwI; Format: bl
ttggccactccctcgatccgcgctcgctcgctcactgaggccgggcgaccaaaggtcgcccgacgcccgggctttgcccgggcggcctcagtgagcgagcgagcgcggatcgagggagtggccaa

SEQ ID NO: 107 - DNA, 125 bp, Synthetic construct
  source 1..125: mol_type = other DNA; note = source: AAV2; Recogn. Site: Nt.AlwI; Format: tl
ttggccactggatctctgcgcgctcgctcgctcactgaggccgggcgaccaaaggtcgcccgacgcccgggctttgcccgggcggcctcagtgagcgagcgagcgcgcagagatccagtggccaa

SEQ ID NO: 108 - DNA, 125 bp, Synthetic construct
  source 1..125: mol_type = other DNA; note = source: AAV3; Recogn. Site: Nt.AlwI; Format: bl
ttggccactccctcgatccgcactcgctcgctcggtggggcctggcgaccaaaggtcgccagacggacgtgctttgcacgtccggccccaccgagcgagcgagtgcggatcgagggagtggccaa

SEQ ID NO: 109 - DNA, 125 bp, Synthetic construct
  source 1..125: mol_type = other DNA; note = source: AAV3; Recogn. Site: Nt.AlwI; Format: tl
ttggccactggatctatgcgcactcgctcgctcggtggggcctggcgaccaaaggtcgccagacggacgtgctttgcacgtccggccccaccgagcgagcgagtgcgcatagatccagtggccaa

SEQ ID NO: 110 - DNA, 125 bp, Synthetic construct
  source 1..125: mol_type = other DNA; note = source: AAV4 left; Recogn. Site: Nt.AlwI; Format: bl
ttggccactccctcgatccgcgctcgctcactcactcggccctggagaccaaaggtctccagactgccggcctctggccggcagggccgagtgagtgagcgagcgcggatcgagggagtggccaa

SEQ ID NO: 111 - DNA, 125 bp, Synthetic construct
  source 1..125: mol_type = other DNA; note = source: AAV4 left; Recogn. Site: Nt.AlwI; Format: tl
ttggccactggatctatgcgcgctcgctcactcactcggccctggagaccaaaggtctccagactgccggcctctggccggcagggccgagtgagtgagcgagcgcgcatagatccagtggccaa

SEQ ID NO: 112 - DNA, 125 bp, Synthetic construct
  source 1..125: mol_type = other DNA; note = source: AAV4 right; Recogn. Site: Nt.AlwI; Format: bl
ttggccacattagcgatccgcgctcgctcactcactcggccctggagaccaaaggtctccagactgccggcctctggccggcagggccgagtgagtgagcgagcgcggatcgagggagtggccaa

SEQ ID NO: 113 - DNA, 125 bp, Synthetic construct
  source 1..125: mol_type = other DNA; note = source: AAV4 right; Recogn. Site: Nt.AlwI; Format: tl
ttggccacaggatctatgcgcgctcgctcactcactcggccctggagaccaaaggtctccagactgccggcctctggccggcagggccgagtgagtgagcgagcgcgcatagatccagtggccaa

SEQ ID NO: 114 - DNA, 137 bp, Synthetic construct
  source 1..137: mol_type = other DNA; note = source: AAV5; Recogn. Site: Nt.AlwI; Format: bl
ctctcccccctgtcgcgatccctcgctcgctggctcgtttgggggggtggcagctcaaagagctgccagacgacggccctctggccgtcgcccccccaaacgagccagcgagcgagggatcgcgacaggggggagag

SEQ ID NO: 115 - DNA, 137 bp, Synthetic construct
  source 1..137: mol_type = other DNA; note = source: AAV5; Recogn. Site: Nt.AlwI; Format: tl
ctctcccccggatcgcgttcgctcgctcgctggctcgtttgggggggtggcagctcaaagagctgccagacgacggccctctggccgtcgcccccccaaacgagccagcgagcgagcgaacgcgatccgggggagag

SEQ ID NO: 116 - DNA, 125 bp, Synthetic construct
  source 1..125: mol_type = other DNA; note = source: AAV7; Recogn. Site: Nt.AlwI; Format: bl
ttggccactccctcgatccgcgctcgctcgctcggtggggcctgcggaccaaaggtccgcagacggcagagctctgctctgccggccccaccgagcgagcgagcgcggatcgagggagtggccaa

SEQ ID NO: 117 - DNA, 125 bp, Synthetic construct
  source 1..125: mol_type = other DNA; note = source: AAV7; Recogn. Site: Nt.AlwI; Format: tl
ttggccactggatctatgcgcgctcgctcgctcggtggggcctgcggaccaaaggtccgcagacggcagagctctgctctgccggccccaccgagcgagcgagcgcgcatagatccagtggccaa

SEQ ID NO: 118 - DNA, 125 bp, Synthetic construct; identical to SEQ ID NO: 2
  source 1..125: mol_type = other DNA; note = source: AAV1; Recogn. Site: Nt.BbvCI; Format: bl

SEQ ID NO: 119 - DNA, 125 bp, Synthetic construct; identical to SEQ ID NO: 1
  source 1..125: mol_type = other DNA; note = source: AAV1; Recogn. Site: Nt.BbvCI; Format: tl

SEQ ID NO: 120 - DNA, 125 bp, Synthetic construct; identical to SEQ ID NO: 37
  source 1..125: mol_type = other DNA; note = source: AAV2; Recogn. Site: Nt.BbvCI; Format: bl

SEQ ID NO: 121 - DNA, 125 bp, Synthetic construct; identical to SEQ ID NO: 36
  source 1..125: mol_type = other DNA; note = source: AAV2; Recogn. Site: Nt.BbvCI; Format: tl

SEQ ID NO: 122 - DNA, 125 bp, Synthetic construct; identical to SEQ ID NO: 39
  source 1..125: mol_type = other DNA; note = source: AAV3; Recogn. Site: Nt.BbvCI; Format: bl

SEQ ID NO: 123 - DNA, 125 bp, Synthetic construct; identical to SEQ ID NO: 38
  source 1..125: mol_type = other DNA; note = source: AAV3; Recogn. Site: Nt.BbvCI; Format: tl

SEQ ID NO: 124 - DNA, 125 bp, Synthetic construct; identical to SEQ ID NO: 41
  source 1..125: mol_type = other DNA; note = source: AAV4 left; Recogn. Site: Nt.BbvCI; Format: bl

SEQ ID NO: 125 - DNA, 125 bp, Synthetic construct; identical to SEQ ID NO: 40
  source 1..125: mol_type = other DNA; note = source: AAV4 left; Recogn. Site: Nt.BbvCI; Format: tl

SEQ ID NO: 126 - DNA, 125 bp, Synthetic construct; identical to SEQ ID NO: 43
  source 1..125: mol_type = other DNA; note = source: AAV4 right; Recogn. Site: Nt.BbvCI; Format: bl

SEQ ID NO: 127 - DNA, 125 bp, Synthetic construct; identical to SEQ ID NO: 42
  source 1..125: mol_type = other DNA; note = source: AAV4 right; Recogn. Site: Nt.BbvCI; Format: tl

SEQ ID NO: 128 - DNA, 137 bp, Synthetic construct; identical to SEQ ID NO: 45
  source 1..137: mol_type = other DNA; note = source: AAV5; Recogn. Site: Nt.BbvCI; Format: bl

SEQ ID NO: 129 - DNA, 137 bp, Synthetic construct; identical to SEQ ID NO: 44
  source 1..137: mol_type = other DNA; note = source: AAV5; Recogn. Site: Nt.BbvCI; Format: tl

SEQ ID NO: 130 - DNA, 125 bp, Synthetic construct; identical to SEQ ID NO: 47
  source 1..125: mol_type = other DNA; note = source: AAV7; Recogn. Site: Nt.BbvCI; Format: bl

SEQ ID NO: 131 - DNA, 125 bp, Synthetic construct; identical to SEQ ID NO: 46
  source 1..125: mol_type = other DNA; note = source: AAV7; Recogn. Site: Nt.BbvCI; Format: tl

SEQ ID NO: 132 - DNA, 125 bp, Synthetic construct
  source 1..125: mol_type = other DNA; note = source: AAV1; Recogn. Site: Nt.BsmAI; Format: bl
ttgcccactgagactctgcgcgctcgctcgctcggtggggcctgcggaccaaaggtccgcagacggcagagctctgctctgccggccccaccgagcgagcgagcgcgcagagtctcagtgggcaa

SEQ ID NO: 133 - DNA, 125 bp, Synthetic construct
  source 1..125: mol_type = other DNA; note = source: AAV1; Recogn. Site: Nt.BsmAI; Format: tl
ttgcccactccgtctctgcgcgctcgctcgctcggtggggcctgcggaccaaaggtccgcagacggcagagctctgctctgccggccccaccgagcgagcgagcgcgcagagacggagtgggcaa

SEQ ID NO: 134 - DNA, 125 bp, Synthetic construct
  source 1..125: mol_type = other DNA; note = source: AAV2; Recogn. Site: Nt.BsmAI; Format: bl
ttggccactgagactctgcgcgctcgctcgctcactgaggccgggcgaccaaaggtcgcccgacgcccgggctttgcccgggcggcctcagtgagcgagcgagcgcgcagagtctcagtggccaa

SEQ ID NO: 135 - DNA, 125 bp, Synthetic construct
  source 1..125: mol_type = other DNA; note = source: AAV2; Recogn. Site: Nt.BsmAI; Format: tl
ttggccactccgtctctgcgcgctcgctcgctcactgaggccgggcgaccaaaggtcgcccgacgcccgggctttgcccgggcggcctcagtgagcgagcgagcgcgcagagacggagtggccaa

SEQ ID NO: 136 - DNA, 125 bp, Synthetic construct
  source 1..125: mol_type = other DNA; note = source: AAV3; Recogn. Site: Nt.BsmAI; Format: bl
ttggccactgagactatgcgcactcgctcgctcggtggggcctggcgaccaaaggtcgccagacggacgtgctttgcacgtccggccccaccgagcgagcgagtgcgcatagtctcagtggccaa

SEQ ID NO: 137 - DNA, 125 bp, Synthetic construct
  source 1..125: mol_type = other DNA; note = source: AAV3; Recogn. Site: Nt.BsmAI; Format: tl
ttggccactccgtctctgcgcactcgctcgctcggtggggcctggcgaccaaaggtcgccagacggacgtgctttgcacgtccggccccaccgagcgagcgagtgcgcagagacggagtggccaa

SEQ ID NO: 138 - DNA, 125 bp, Synthetic construct
  source 1..125: mol_type = other DNA; note = source: AAV4 left; Recogn. Site: Nt.BsmAI; Format: bl
ttggccactgagactatgcgcgctcgctcactcactcggccctggagaccaaaggtctccagactgccggcctctggccggcagggccgagtgagtgagcgagcgcgcatagtctcagtggccaa

SEQ ID NO: 139 - DNA, 125 bp, Synthetic construct
  source 1..125: mol_type = other DNA; note = source: AAV4 left; Recogn. Site: Nt.BsmAI; Format: tl
ttggccactccgtctctgcgcgctcgctcactcactcggccctggagaccaaaggtctccagactgccggcctctggccggcagggccgagtgagtgagcgagcgcgcagagacggagtggccaa

SEQ ID NO: 140 - DNA, 125 bp, Synthetic construct
  source 1..125: mol_type = other DNA; note = source: AAV4 right; Recogn. Site: Nt.BsmAI; Format: bl
ttggccacagagactatgcgcgctcgctcactcactcggccctggagaccaaaggtctccagactgccggcctctggccggcagggccgagtgagtgagcgagcgcgcatagtctcagtggccaa

SEQ ID NO: 141 - DNA, 125 bp, Synthetic construct
  source 1..125: mol_type = other DNA; note = source: AAV4 right; Recogn. Site: Nt.BsmAI; Format: tl
ttggccacattgtctctgcgcgctcgctcactcactcggccctggagaccaaaggtctccagactgccggcctctggccggcagggccgagtgagtgagcgagcgcgcagagacggagtggccaa

SEQ ID NO: 142 - DNA, 137 bp, Synthetic construct
  source 1..137: mol_type = other DNA; note = source: AAV5; Recogn. Site: Nt.BsmAI; Format: bl
ctctcccccgagacgcgttcgctcgctcgctggctcgtttgggggggtggcagctcaaagagctgccagacgacggccctctggccgtcgcccccccaaacgagccagcgagcgagcgaacgcgtctcgggggagag

SEQ ID NO: 143 - DNA, 137 bp, Synthetic construct
  source 1..137: mol_type = other DNA; note = source: AAV5; Recogn. Site: Nt.BsmAI; Format: tl
ctctcccccgtctcgcgttcgctcgctcgctggctcgtttgggggggtggcagctcaaagagctgccagacgacggccctctggccgtcgcccccccaaacgagccagcgagcgagcgaacgcgagacgggggagag

SEQ ID NO: 144 - DNA, 125 bp, Synthetic construct
  source 1..125: mol_type = other DNA; note = source: AAV7; Recogn. Site: Nt.BsmAI; Format: bl
ttggccactgagactatgcgcgctcgctcgctcggtggggcctgcggaccaaaggtccgcagacggcagagctctgctctgccggccccaccgagcgagcgagcgcgcatagtctcagtggccaa

SEQ ID NO: 145 - DNA, 125 bp, Synthetic construct
  source 1..125: mol_type = other DNA; note = source: AAV7; Recogn. Site: Nt.BsmAI; Format: tl
ttggccactccgtctctgcgcgctcgctcgctcggtggggcctgcggaccaaaggtccgcagacggcagagctctgctctgccggccccaccgagcgagcgagcgcgcagagacggagtggccaa

SEQ ID NO: 146 - DNA, 125 bp, Synthetic construct
  source 1..125: mol_type = other DNA; note = source: AAV1; Recogn. Site: Nt.BspQI; Format: bl
ttgcccactcccgaagagcgcgctcgctcgctcggtggggcctgcggaccaaaggtccgcagacggcagagctctgctctgccggccccaccgagcgagcgagcgcgctcttcgggagtgggcaa

SEQ ID NO: 147 - DNA, 125 bp, Synthetic construct
  source 1..125: mol_type = other DNA; note = source: AAV1; Recogn. Site: Nt.BspQI; Format: tl
ttgcccactcccgctcttcgcgctcgctcgctcggtggggcctgcggaccaaaggtccgcagacggcagagctctgctctgccggccccaccgagcgagcgagcgcgaagagcgggagtgggcaa

SEQ ID NO: 148 - DNA, 125 bp, Synthetic construct
  source 1..125: mol_type = other DNA; note = source: AAV2; Recogn. Site: Nt.BspQI; Format: bl
ttggccactcccgaagagcgcgctcgctcgctcactgaggccgggcgaccaaaggtcgcccgacgcccgggctttgcccgggcggcctcagtgagcgagcgagcgcgctcttcgggagtggccaa

SEQ ID NO: 149 - DNA, 125 bp, Synthetic construct
  source 1..125: mol_type = other DNA; note = source: AAV2; Recogn. Site: Nt.BspQI; Format: tl
ttggccactcccgctcttcgcgctcgctcgctcactgaggccgggcgaccaaaggtcgcccgacgcccgggctttgcccgggcggcctcagtgagcgagcgagcgcgaagagcgggagtggccaa

SEQ ID NO: 150 - DNA, 125 bp, Synthetic construct
  source 1..125: mol_type = other DNA; note = source: AAV3; Recogn. Site: Nt.BspQI; Format: bl
ttggccactcccgaagagcgcactcgctcgctcggtggggcctggcgaccaaaggtcgccagacggacgtgctttgcacgtccggccccaccgagcgagcgagtgcgctcttcgggagtggccaa

SEQ ID NO: 151 - DNA, 125 bp, Synthetic construct
  source 1..125: mol_type = other DNA; note = source: AAV3; Recogn. Site: Nt.BspQI; Format: tl
ttggccactcccgctcttcgcactcgctcgctcggtggggcctggcgaccaaaggtcgccagacggacgtgctttgcacgtccggccccaccgagcgagcgagtgcgaagagcgggagtggccaa

SEQ ID NO: 152 - DNA, 125 bp, Synthetic construct
  source 1..125: mol_type = other DNA; note = source: AAV4 left; Recogn. Site: Nt.BspQI; Format: bl
ttggccactcccgaagagcgcgctcgctcactcactcggccctggagaccaaaggtctccagactgccggcctctggccggcagggccgagtgagtgagcgagcgcgctcttcgggagtggccaa

SEQ ID NO: 153 - DNA, 125 bp, Synthetic construct
  source 1..125: mol_type = other DNA; note = source: AAV4 left; Recogn. Site: Nt.BspQI; Format: tl
ttggccactcccgctcttcgcgctcgctcactcactcggccctggagaccaaaggtctccagactgccggcctctggccggcagggccgagtgagtgagcgagcgcgaagagcgggagtggccaa

SEQ ID NO: 154 - DNA, 125 bp, Synthetic construct
  source 1..125: mol_type = other DNA; note = source: AAV4 right; Recogn. Site: Nt.BspQI; Format: bl
ttggccacattagaagagcgcgctcgctcactcactcggccctggagaccaaaggtctccagactgccggcctctggccggcagggccgagtgagtgagcgagcgcgctcttcgggagtggccaa

SEQ ID NO: 155 - DNA, 125 bp, Synthetic construct
  source 1..125: mol_type = other DNA; note = source: AAV4 right; Recogn. Site: Nt.BspQI; Format: tl
ttggccacattagctcttcgcgctcgctcactcactcggccctggagaccaaaggtctccagactgccggcctctggccggcagggccgagtgagtgagcgagcgcgaagagcgggagtggccaa

SEQ ID NO: 156 - DNA, 137 bp, Synthetic construct
  source 1..137: mol_type = other DNA; note = source: AAV5; Recogn. Site: Nt.BspQI; Format: bl
ctctcccgaagagcgcgttcgctcgctcgctggctcgtttgggggggtggcagctcaaagagctgccagacgacggccctctggccgtcgcccccccaaacgagccagcgagcgagcgaacgcgctcttcgggagag

SEQ ID NO: 157 - DNA, 137 bp, Synthetic construct
  source 1..137: mol_type = other DNA; note = source: AAV5; Recogn. Site: Nt.BspQI; Format: tl
ctctcccgctcttcgcgttcgctcgctcgctggctcgtttgggggggtggcagctcaaagagctgccagacgacggccctctggccgtcgcccccccaaacgagccagcgagcgagcgaacgcgaagagcgggagag

SEQ ID NO: 158 - DNA, 125 bp, Synthetic construct
  source 1..125: mol_type = other DNA; note = source: AAV7; Recogn. Site: Nt.BspQI; Format: bl
ttggccactcccgaagagcgcgctcgctcgctcggtggggcctgcggaccaaaggtccgcagacggcagagctctgctctgccggccccaccgagcgagcgagcgcgctcttcgggagtggccaa

SEQ ID NO: 159 - DNA, 125 bp, Synthetic construct
  source 1..125: mol_type = other DNA; note = source: AAV7; Recogn. Site: Nt.BspQI; Format: tl
ttggccactcccgctcttcgcgctcgctcgctcggtggggcctgcggaccaaaggtccgcagacggcagagctctgctctgccggccccaccgagcgagcgagcgcgaagagcgggagtggccaa

SEQ ID NO: 160 - DNA, 127 bp, Synthetic construct
  source 1..127: mol_type = other DNA; note = source: AAV1; Recogn. Site: Nt.BstNBI; Format: bl
ttgcccactccctctctgcgcgactcgctcgctcggtggggcctgcggaccaaaggtccgcagacggcagagctctgctctgccggccccaccgagcgagcgagtcgcgcagagagggagtgggcaa

SEQ ID NO: 161 - DNA, 125 bp, Synthetic construct
  source 1..125: mol_type = other DNA; note = source: AAV1; Recogn. Site: Nt.BstNBI; Format: tl
ttgccgagtccctctctgcgcgctcgctcgctcggtggggcctgcggaccaaaggtccgcagacggcagagctctgctctgccggccccaccgagcgagcgagcgcgcagagagggactcggcaa

SEQ ID NO: 162 - DNA, 127 bp, Synthetic construct
  source 1..127: mol_type = other DNA; note = source: AAV2; Recogn. Site: Nt.BstNBI; Format: bl
ttggccactccctctctgcgcgactcgctcgctcactgaggccgggcgaccaaaggtcgcccgacgcccgggctttgcccgggcggcctcagtgagcgagcgagtcgcgcagagagggagtggccaa

SEQ ID NO: 163 - DNA, 125 bp, Synthetic construct
  source 1..125: mol_type = other DNA; note = source: AAV2; Recogn. Site: Nt.BstNBI; Format: tl
ttggcgagtccctctctgcgcgctcgctcgctcactgaggccgggcgaccaaaggtcgcccgacgcccgggctttgcccgggcggcctcagtgagcgagcgagcgcgcagagagggactcgccaa

SEQ ID NO: 164 - DNA, 127 bp, Synthetic construct
  source 1..127: mol_type = other DNA; note = source: AAV3; Recogn. Site: Nt.BstNBI; Format: bl
ttggccactccctctatgcgcgactcgctcgctcggtggggcctggcgaccaaaggtcgccagacggacgtgctttgcacgtccggccccaccgagcgagcgagtcgcgcatagagggagtggccaa

SEQ ID NO: 165 - DNA, 125 bp, Synthetic construct
  source 1..125: mol_type = other DNA; note = source: AAV3; Recogn. Site: Nt.BstNBI; Format: tl
ttggcgagtccctctatgcgcactcgctcgctcggtggggcctggcgaccaaaggtcgccagacggacgtgctttgcacgtccggccccaccgagcgagcgagtgcgcatagagggactcgccaa

SEQ ID NO: 166 - DNA, 127 bp, Synthetic construct
  source 1..127: mol_type = other DNA; note = source: AAV4 left; Recogn. Site: Nt.BstNBI; Format: bl
ttggccactccctctatgcgcgactcgctcactcactcggccctggagaccaaaggtctccagactgccggcctctggccggcagggccgagtgagtgagcgagtcgcgcatagagggagtggccaa

SEQ ID NO: 167 - DNA, 125 bp, Synthetic construct
  source 1..125: mol_type = other DNA; note = source: AAV4 left; Recogn. Site: Nt.BstNBI; Format: tl
ttggcgagtccctctatgcgcgctcgctcactcactcggccctggagaccaaaggtctccagactgccggcctctggccggcagggccgagtgagtgagcgagcgcgcatagagggactcgccaa

SEQ ID NO: 168 - DNA, 127 bp, Synthetic construct
  source 1..127: mol_type = other DNA; note = source: AAV4 right; Recogn. Site: Nt.BstNBI; Format: bl
ttggccacattagctatgcgcgactcgctcactcactcggccctggagaccaaaggtctccagactgccggcctctggccggcagggccgagtgagtgagcgagtcgcgcatagagggagtggccaa

SEQ ID NO: 169 - DNA, 125 bp, Synthetic construct
  source 1..125: mol_type = other DNA; note = source: AAV4 right; Recogn. Site: Nt.BstNBI; Format: tl
ttggccagagtcgctatgcgcgctcgctcactcactcggccctggagaccaaaggtctccagactgccggcctctggccggcagggccgagtgagtgagcgagcgcgcatagagactctggccaa

SEQ ID NO: 170 - DNA, 139 bp, Synthetic construct
  source 1..139: mol_type = other DNA; note = source: AAV5; Recogn. Site: Nt.BstNBI; Format: bl
ctctcccccctgtcgcgactcgctcgctcgctggctcgtttgggggggtggcagctcaaagagctgccagacgacggccctctggccgtcgcccccccaaacgagccagcgagcgagcgagtcgcgacaggggggagag

SEQ ID NO: 171 - DNA, 137 bp, Synthetic construct
  source 1..137: mol_type = other DNA; note = source: AAV5; Recogn. Site: Nt.BstNBI; Format: tl
ctctcccccgagtcgcgttcgctcgctcgctggctcgtttgggggggtggcagctcaaagagctgccagacgacggccctctggccgtcgcccccccaaacgagccagcgagcgagcgaacgcgactcgggggagag

SEQ ID NO: 172 - DNA, 127 bp, Synthetic construct
  source 1..127: mol_type = other DNA; note = source: AAV7; Recogn. Site: Nt.BstNBI; Format: bl
ttggccactccctctatgcgcgactcgctcgctcggtggggcctgcggaccaaaggtccgcagacggcagagctctgctctgccggccccaccgagcgagcgagtcgcgcatagagggagtggccaa

SEQ ID NO: 173 - DNA, 125 bp, Synthetic construct
  source 1..125: mol_type = other DNA; note = source: AAV7; Recogn. Site: Nt.BstNBI; Format: tl
ttggcgagtccctctatgcgcgctcgctcgctcggtggggcctgcggaccaaaggtccgcagacggcagagctctgctctgccggccccaccgagcgagcgagcgcgcatagagggactcgccaa

SEQ ID NO: 174 - DNA, 7056 bp, Homo sapiens
  source 1..7056: mol_type = genomic DNA; note = Native FVIII isoform a
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagaattcaagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgcgacagagtcctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagtcaggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagtctggtggacctctgagcttgagtgaagaaaataatgattcaaagttgttagaatcaggtttaatgaatagccaagaaagttcatggggaaaaaatgtatcgtcaacagagagtggtaggttatttaaagggaaaagagctcatggacctgctttgttgactaaagataatgccttattcaaagttagcatctctttgttaaagacaaacaaaacttccaataattcagcaactaatagaaagactcacattgatggcccatcattattaattgagaatagtccatcagtctggcaaaatatattagaaagtgacactgagtttaaaaaagtgacacctttgattcatgacagaatgcttatggacaaaaatgctacagctttgaggctaaatcatatgtcaaataaaactacttcatcaaaaaacatggaaatggtccaacagaaaaaagagggccccattccaccagatgcacaaaatccagatatgtcgttctttaagatgctattcttgccagaatcagcaaggtggatacaaaggactcatggaaagaactctctgaactctgggcaaggccccagtccaaagcaattagtatccttaggaccagaaaaatctgtggaaggtcagaatttcttgtctgagaaaaacaaagtggtagtaggaaagggtgaatttacaaaggacgtaggactcaaagagatggtttttccaagcagcagaaacctatttcttactaacttggataatttacatgaaaataatacacacaatcaagaaaaaaaaattcaggaagaaatagaaaagaaggaaacattaatccaagagaatgtagttttgcctcagatacatacagtgactggcactaagaatttcatgaagaaccttttcttactgagcactaggcaaaatgtagaaggttcatatgacggggcatatgctccagtacttcaagattttaggtcattaaatgattcaacaaatagaacaaagaaacacacagctcatttctcaaaaaaaggggaggaagaaaacttggaaggcttgggaaatcaaaccaagcaaattgtagagaaatatgcatgcaccacaaggatatctcctaatacaagccagcagaattttgtcacgcaacgtagtaagagagctttgaaacaattcagactcccactagaagaaacagaacttgaaaaaaggataattgtggatgacacctcaacccagtggtccaaaaacatgaaacatttgaccccgagcaccctcacacagatagactacaatgagaaggagaaaggggccattactcagtctcccttatcagattgccttacgaggagtcatagcatccctcaagcaaatagatctccattacccattgcaaaggtatcatcatttccatctattagacctatatatctgaccagggtcctattccaagacaactcttctcatcttccagcagcatcttatagaaagaaagattctggggtccaagaaagcagtcatttcttacaaggagccaaaaaaaataacctttctttagccattctaaccttggagatgactggtgatcaaagagaggttggctccctggggacaagtgccacaaattcagtcacatacaagaaagttgagaacactgttctcccgaaaccagacttgcccaaaacatctggcaaagttgaattgcttccaaaagttcacatttatcagaaggacctattccctacggaaactagcaatgggtctcctggccatctggatctcgtggaagggagccttcttcagggaacagagggagcgattaagtggaatgaagcaaacagacctggaaaagttccctttctgagagtagcaacagaaagctctgcaaagactccctccaagctattggatcctcttgcttgggataaccactatggtactcagataccaaaagaagagtggaaatcccaagagaagtcaccagaaaaaacagcttttaagaaaaaggataccattttgtccctgaacgcttgtgaaagcaatcatgcaatagcagcaataaatgagggacaaaataagcccgaaatagaagtcacctgggcaaagcaaggtaggactgaaaggctgtgctctcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 175 - DNA, 7056 bp, Synthetic construct
  source 1..7056: mol_type = other DNA; note = FVIII v5
atgcagattgaactgtctacctgcttctttctgtgcctcctgaggttctgttttagtgccacaaggagatattacctaggagcagttgagctgtcctgggattacatgcagagtgatctgggggagctgccagttgatgctaggttccctccaagggtgcctaagtccttcccctttaatacctcagtggtgtataagaagaccctttttgtagagttcactgatcacctgttcaatatagctaaacccaggccaccttggatggggttgttgggcccaacaattcaggcagaggtttatgacacagtagtgataaccctcaagaatatggctagtcatcctgtgagcctgcatgcagtaggtgtgtcttactggaaggcttcagagggtgctgagtatgatgatcagacttctcagagagagaaggaagatgacaaagtctttccagggggatcacatacatatgtttggcaggttcttaaagagaatggtcctatggccagtgaccccctgtgtctgacttactcctacctgagccatgtggacctggtgaaggatctgaactctgggctgataggtgcactgctggtgtgtagggaaggtagccttgccaaagagaagacccaaacactccacaagttcatcttgctctttgcagtttttgatgaaggaaaaagctggcatagtgaaaccaaaaactctttgatgcaggatagggatgcagctagtgctagggcctggccaaagatgcacacagtcaatggttatgtgaacagaagtcttccaggtctgataggatgccacaggaaaagtgtgtactggcatgtgataggcatgggaaccaccccagaagtgcactcaatattccttgagggtcataccttccttgtgagaaatcataggcaggcatccttggagatcagtcccatcaccttcttaactgcccaaacccttctgatggaccttggtcaatttctgctgttttgccatatttcttcccatcagcatgatgggatggaggcctatgtaaaggtggattcttgcccagaggaaccccagctgaggatgaagaacaatgaggaagcagaggattatgatgatgatcttactgatagtgagatggatgtggttaggtttgatgatgacaacagccccagctttatccagattaggtctgttgctaagaagcatccaaagacatgggttcattacatagctgcagaagaagaggactgggattatgcccccctagtccttgccccagatgatagatcctataaatctcagtatctgaacaatggacctcagaggataggcaggaagtataagaaagtgagattcatggcatatacagatgagaccttcaagaccagagaagcaatccaacatgaaagtgggattctgggcccactgctgtatggtgaggtgggtgacaccctgctgataatcttcaagaatcaggccagcaggccttataacatctatccacatgggataacagatgtaaggcctctgtatagtagaaggctgcccaaaggggtgaaacacctgaaagactttcctattcttccaggtgaaatcttcaaatacaagtggacagtcacagtggaggatgggcctactaaaagtgaccccaggtgcctgactaggtactatagttcctttgttaacatggaaagggacctagctagtgggcttataggcccccttctgatctgttataaagaatctgttgatcagaggggcaaccagatcatgtcagacaaaaggaatgtgatactttttagtgtgtttgatgagaataggagctggtacctgactgagaacatccagaggtttttgcccaacccagctggtgtgcagcttgaggacccagagtttcaagcctcaaacatcatgcacagtatcaatggctatgtttttgattctcttcagctgagtgtttgcctgcatgaagtagcttactggtacatcctctcaattggggctcagacagatttcctttcagtgttcttcagtggctacactttcaagcacaaaatggtctatgaggacactctgaccttattcccttttagtggagagacagtctttatgagtatggagaatcctgggctgtggattcttgggtgccacaactctgatttcagaaataggggaatgactgccctgctaaaggtgagtagttgtgacaaaaacacaggggattactatgaggacagctatgaggacatcagtgcctatctgctctcaaagaacaatgcaatagagcctaggtccttctcacagaatagcaggcatccctcaaccaggcaaaaacagtttaatgccaccaccataccagagaatgatattgaaaagactgacccttggtttgctcacaggaccccaatgcccaaaatccaaaatgtgtcttcaagtgacctgttaatgctgttgaggcagtcacccaccccacatgggctgtccctgtcagatcttcaagaggccaaatatgagaccttcagtgatgatccttcaccaggagccatagattctaacaactccctgtcagaaatgacacacttcagacctcagttacatcattctggggatatggtgttcacacctgaatctggtctccagctgaggttaaatgagaaacttgggactacagcagctactgagctgaaaaaacttgattttaaggtgagctcaacaagcaataacctgatttccaccattcctagtgacaacctagctgctggcactgataacacctcttctctgggccctccttcaatgccagtacattatgattcccaacttgataccaccctctttgggaagaagtctagcccacttacagaatcaggtggcccacttagtctctcagaggagaataatgattccaaacttttggagagtgggctgatgaactctcaagagagcagttgggggaagaatgtatcctccacagaaagtgggaggttgtttaaggggaagagagctcatggacctgctctgctgaccaaggacaatgctcttttcaaggtttccatttccctcctgaaaacaaataagacttccaacaactcagcaactaataggaaaacacatatagatggacctagcctgctgattgaaaacagcccctcagtgtggcagaacatattggaatctgatacagagttcaagaaggtgacccctctcatccatgataggatgctgatggataagaatgccactgctttgaggctcaatcacatgtctaataaaactacctccagtaaaaacatggagatggtccagcagaaaaaggagggtcccatacctcctgatgcccagaatccagatatgtctttctttaaaatgttgttcctgccagaatctgctaggtggatacaaaggacacatggaaaaaattccttgaactctggacagggccctagtcctaaacagttagttagcttgggtccagagaagagtgtagaagggcagaatttcctcagtgagaagaacaaggtggttgtaggcaagggagagttcacaaaggatgttggccttaaggaaatggtattccctagcagcaggaacttgtttcttaccaacctggataatctgcatgaaaacaacactcacaaccaagagaagaaaattcaggaggagatagagaaaaaggagaccctcattcaggaaaatgtggtgctgccacagattcacacagtaacaggcacaaagaatttcatgaagaatctttttttgttgtcaactaggcagaatgtggagggctcctatgatggggcctatgcaccagtactgcaagatttcaggtccctcaatgacagcacaaataggaccaaaaaacatactgcccacttttccaaaaaaggggaagaggagaatctggaggggctggggaatcagaccaagcagatagtagaaaaatatgcttgtactaccagaatcagccctaacacttcccagcagaattttgtgacccagagaagcaaaagggctctgaagcagtttaggctccccctggaggagactgagttggaaaaaagaatcatagtagatgacacctcaacccaatggtctaagaacatgaagcacttgaccccttccactctcacacagatagattataatgagaaagagaagggagctatcacacaatcccccctcagtgattgtctcactaggtcacactctatccctcaggctaatagaagcccactcccaattgccaaggtgtcatcattcccttctatcaggcctatatatttaacaagggttttgttccaggataattcatctcatctcccagcagcctcttataggaagaaggattcaggggtccaagaatccagccactttctgcagggagctaagaagaacaacttgtcccttgctatattgaccctggagatgactggggatcagagggaggtaggttctttggggacctcagccactaactctgtaacatacaaaaaggtagaaaatacagtgttaccaaaaccagatcttccaaagacctcaggaaaagtggaactgctccctaaagtgcacatttaccagaaggatttgtttcctactgagacaagtaatggaagccctgggcacctagacctggtggagggcagtcttctccaggggacagagggagccattaaatggaatgaggccaacagaccaggtaaggttccatttctgagggttgctacagaatccagtgccaagaccccaagtaaactgctggacccattggcatgggacaaccattatggcacccaaattcccaaggaggaatggaagtctcaggaaaagtcacctgaaaagacagctttcaagaaaaaagacaccattttgagtttgaatgcttgtgaatccaatcatgccatagctgctattaatgagggacagaacaaacctgaaattgaggttacatgggcaaagcagggaagaactgagagactgtgctctcagaaccctccagttttgaaaaggcaccagagagagataaccagaacaacccttcagtctgaccaggaggagatagattatgatgacactatctcagttgagatgaaaaaggaggactttgatatctatgatgaagatgaaaatcagtctcccaggtccttccagaaaaaaacaaggcattacttcatagcagcagtagagagactgtgggattatgggatgtcatccagtcctcatgtcctgaggaacagagctcagagtggctcagtgcctcaattcaagaaagtggtgttccaggagttcacagatggctccttcacacagcctctctacagaggagagctcaatgaacacttggggctgctggggccctatatcagggcagaggtggaggataatattatggtgacatttaggaaccaagcctcaaggccatactcattttattcttccctgatctcatatgaagaggatcagaggcagggggcagaacctaggaagaattttgtaaagcctaatgagactaagacttacttttggaaggtgcaacaccacatggctcccaccaaagatgaatttgattgcaaggcttgggcctacttctctgatgtggatctggagaaagatgtgcattctggcttgataggtcccctactggtttgtcatacaaataccctgaaccctgcacatggcaggcaggtgactgtgcaggagtttgctctcttcttcactatatttgatgagaccaagtcctggtacttcacagagaatatggaaagaaactgcagggccccatgtaatatccagatggaagaccccacctttaaggaaaattataggtttcatgctattaatggatatataatggacaccctcccaggtctggtcatggcacaggaccagaggataaggtggtatctgctttctatgggctctaatgaaaatattcatagcatccactttagtgggcatgtgttcactgtcaggaaaaaagaggagtataaaatggccctgtataacctgtacccaggagtgtttgagacagtagagatgcttcctagtaaggctggcatttggagagtagagtgcctgattggagaacatctccatgctggaatgagcaccctgttcctggtctatagtaacaagtgtcagaccccactgggcatggcctcaggtcatattagggacttccaaatcactgccagtggccaatatgggcagtgggcccccaaactagccaggttgcactattctgggagtattaatgcctggtctacaaaggaaccattttcctggatcaaagtggatctgctggccccaatgatcatacatggaattaagacccagggagccaggcagaagttttcctccctgtacattagtcagttcatcattatgtacagtttggatgggaaaaagtggcagacatataggggtaacagtacaggaacccttatggtgttctttggtaatgttgacagctcaggaatcaagcacaatattttcaaccccccaatcatagccaggtacatcaggctccaccctactcattatagcatcaggagcaccctgagaatggagcttatgggatgtgacctaaactcctgtagtatgcctctgggaatggaatcaaaggccatctctgatgcccaaattacagcctcatcctactttacaaacatgtttgctacttggtcaccatccaaagctagactgcacctgcaggggaggtcaaatgcctggaggccacaagtcaacaaccccaaagaatggctccaagttgactttcagaagactatgaaagtgactggggtgactactcaaggggtaaagtctcttcttaccagtatgtatgtcaaagagtttttgataagctcctcccaggatggccaccagtggacactgttttttcagaatggcaaggtgaaagtgttccaaggaaaccaggattcttttactccagtggtgaacagcctggaccccccccttctgaccagatacttgagaatccacccccagtcctgggttcatcagattgctctgaggatggaagtgctgggatgtgaggcccaggacctctactaa

SEQ ID NO: 176 - DNA, 4392 bp, Synthetic construct
  source 1..4392: mol_type = other DNA; note = CpG free gene encoding F8 truncation
atgcagattgaactgtctacctgcttctttctgtgcctcctgaggttctgttttagtgccacaaggagatattacctaggagcagttgagctgtcctgggattacatgcagagtgatctgggggagctgccagttgatgctaggttccctccaagggtgcctaagtccttcccctttaatacctcagtggtgtataagaagaccctttttgtagagttcactgatcacctgttcaatatagctaaacccaggccaccttggatggggttgttgggcccaacaattcaggcagaggtttatgacacagtagtgataaccctcaagaatatggctagtcatcctgtgagcctgcatgcagtaggtgtgtcttactggaaggcttcagagggtgctgagtatgatgatcagacttctcagagagagaaggaagatgacaaagtctttccagggggatcacatacatatgtttggcaggttcttaaagagaatggtcctatggccagtgaccccctgtgtctgacttactcctacctgagccatgtggacctggtgaaggatctgaactctgggctgataggtgcactgctggtgtgtagggaaggtagccttgccaaagagaagacccaaacactccacaagttcatcttgctctttgcagtttttgatgaaggaaaaagctggcatagtgaaaccaaaaactctttgatgcaggatagggatgcagctagtgctagggcctggccaaagatgcacacagtcaatggttatgtgaacagaagtcttccaggtctgataggatgccacaggaaaagtgtgtactggcatgtgataggcatgggaaccaccccagaagtgcactcaatattccttgagggtcataccttccttgtgagaaatcataggcaggcatccttggagatcagtcccatcaccttcttaactgcccaaacccttctgatggaccttggtcaatttctgctgagctgccatatttcttcccatcagcatgatgggatggaggcctatgtaaaggtggattcttgcccagaggaaccccagctgaggatgaagaacaatgaggaagcagaggattatgatgatgaccttactgatagtgagatggatgtggttaggtttgatgatgacaacagccccagctttatccagattaggtctgttgctaagaagcatccaaagacatgggttcattacatagctgcagaagaagaggactgggattatgcccccctagtccttgccccagatgatagatcctataaatctcagtatctgaacaatggacctcagaggataggcaggaagtataagaaagtgagattcatggcatatacagatgagaccttcaagaccagagaagcaatccaacatgaaagtgggattctgggcccactgctgtatggtgaggtgggtgacaccctgctgataatcttcaagaatcaggccagcaggccttataacatctatccacatgggataacagatgtaaggcctctgtatagtagaaggctgcccaaaggggtgaaacacctgaaagactttcctattcttccaggtgaaatcttcaaatacaagtggacagtcacagtggaggatgggcctactaaaagtgaccccaggtgcctgactaggtactatagttcctttgttaacatggaaagggacctagctagtgggcttataggcccccttctgatctgttataaagaatctgttgatcagaggggcaaccagatcatgtcagacaaaaggaatgtgatactttttagtgtgtttgatgagaataggagctggtacctgactgagaacatccagaggtttttgcccaacccagctggtgtgcagcttgaggacccagagtttcaagcctcaaacatcatgcacagtatcaatggctatgtttttgattctcttcagctgagtgtttgcctgcatgaagtagcttactggtacatcctctcaattggggctcagacagatttcctttcagtgttcttcagtggctacactttcaagcacaaaatggtctatgaggacactctgaccttattcccttttagtggagagacagtctttatgagtatggagaatcctgggctgtggattcttgggtgccacaactctgatttcagaaataggggaatgactgccctgctaaaggtgagtagttgtgacaaaaacacaggggattactatgaggacagctatgaggacatcagtgcctatctgctctcaaagaacaatgcaatagagcctaggtccttctcacagaatagcaggcatccctcaaccaggcaaaaacagtttaatgccaccaccataccagagaatacaacccttcagtctgaccaggaggagatagattatgatgacactatctcagttgagatgaaaaaggaggactttgatatctatgatgaagatgaaaaccagtctcccaggtccttccagaaaaaaacaaggcattacttcatagcagcagtagagagactgtgggattatgggatgtcatccagtcctcatgtcctgaggaacagagctcagagtggctcagtgcctcaattcaagaaagtggtgttccaggagttcacagatggctccttcacacagcctctctacagaggagagctcaatgaacacttggggctgctggggccctatatcagggcagaggtggaggataatattatggtgacatttaggaaccaagcctcaaggccatactcattttattcttccctgatctcatatgaagaggatcagaggcagggggcagaacctaggaagaattttgtaaagcctaatgagactaagacttacttttggaaggtgcaacaccacatggctcccaccaaagatgaatttgattgcaaggcttgggcctacttctctgatgtggacctggagaaagatgtgcattctggcttgataggtcccctactggtttgtcatacaaataccctgaaccctgcacatggcaggcaggtgactgtgcaggagtttgctctcttcttcactatatttgatgagaccaagtcctggtacttcacagagaatatggaaagaaactgcagggccccatgtaatatccagatggaagaccccacctttaaggaaaattataggtttcatgctattaatggatatataatggacaccctcccaggtctggtcatggcacaggaccagaggataaggtggtatctgctttctatgggctctaatgaaaatattcatagcatccactttagtgggcatgtgttcactgtcaggaaaaaagaggagtataaaatggccctgtataacctgtacccaggagtgtttgagacagtagagatgcttcctagtaaggctggcatttggagagtagagtgcctgattggagaacatctccatgctggaatgagcaccctgttcctggtctatagtaacaagtgtcagaccccactgggcatggcctcaggtcatattagggacttccaaatcactgccagtggccaatatgggcagtgggcccccaaactagccaggttgcactattctgggagtattaatgcctggtctacaaaggaaccattttcctggatcaaagtggatctgctggccccaatgatcatacatggaattaagacccagggagccaggcagaagttttcctccctgtacattagtcagttcatcattatgtacagtttggatgggaaaaagtggcagacatataggggtaacagtacaggaacccttatggtgttctttggtaatgttgacagctcaggaatcaagcacaatattttcaaccccccaatcatagccaggtacatcaggctccaccctactcattatagcatcaggagcaccctgagaatggagcttatgggatgtgacctaaactcctgtagtatgcctctgggaatggaatcaaaggccatctctgatgcccaaattacagcctcatcctactttacaaacatgtttgctacttggtcaccatccaaagctagactgcacctgcaggggaggtcaaatgcctggaggccacaagtcaacaaccccaaagaatggctccaagttgactttcagaagactatgaaagtgactggggtgactactcaaggggtaaagtctcttcttaccagtatgtatgtcaaagagtttttgataagctcctcccaggatggccaccagtggacactgttttttcagaatggcaaggtgaaagtgttccaaggaaaccaggattcttttactccagtggtgaacagcctggaccccccccttctgaccagatacttgagaatccacccccagtcctgggttcatcagattgctctgaggatggaagtgctgggatgtgaggcccaggacctctactag

SEQ ID NO: 177 - skipped

SEQ ID NO: 178 - DNA, 4374 bp, Synthetic construct
  source 1..4374: mol_type = other DNA; note = FVIII v7
atgcagattgaactgtctacctgcttctttctgtgcctcctgaggttctgttttagtgccacaaggagatattacctaggagcagttgagctgtcctgggattacatgcagagtgatctgggggagctgccagttgatgctaggttccctccaagggtgcctaagtccttcccctttaatacctcagtggtgtataagaagaccctttttgtagagttcactgatcacctgttcaatatagctaaacccaggccaccttggatggggttgttgggcccaacaattcaggcagaggtttatgacacagtagtgataaccctcaagaatatggctagtcatcctgtgagcctgcatgcagtaggtgtgtcttactggaaggcttcagagggtgctgagtatgatgatcagacttctcagagagagaaggaagatgacaaagtctttccagggggatcacatacatatgtttggcaggttcttaaagagaatggtcctatggccagtgaccccctgtgtctgacttactcctacctgagccatgtggacctggtgaaggatctgaactctgggctgataggtgcactgctggtgtgtagggaaggtagccttgccaaagagaagacccaaacactccacaagttcatcttgctctttgcagtttttgatgaaggaaaaagctggcatagtgaaaccaaaaactctttgatgcaggatagggatgcagctagtgctagggcctggccaaagatgcacacagtcaatggttatgtgaacagaagtcttccaggtctgataggatgccacaggaaaagtgtgtactggcatgtgataggcatgggaaccaccccagaagtgcactcaatattccttgagggtcataccttccttgtgagaaatcataggcaggcatccttggagatcagtcccatcaccttcttaactgcccaaacccttctgatggaccttggtcaatttctgctgtcctgccatatttcttcccatcagcatgatgggatggaggcctatgtaaaggtggattcttgcccagaggaaccccagctgaggatgaagaacaatgaggaagcagaggattatgatgatgatcttactgatagtgagatggatgtggttaggtttgatgatgacaacagccccagctttatccagattaggtctgttgctaagaagcatccaaagacatgggttcattacatagctgcagaagaagaggactgggattatgcccccctagtccttgccccagatgatagatcctataaatctcagtatctgaacaatggacctcagaggataggcaggaagtataagaaagtgagattcatggcatatacagatgagaccttcaagaccagagaagcaatccaacatgaaagtgggattctgggcccactgctgtatggtgaggtgggtgacaccctgctgataatcttcaagaatcaggccagcaggccttataacatctatccacatgggataacagatgtaaggcctctgtatagtagaaggctgcccaaaggggtgaaacacctgaaagactttcctattcttccaggtgaaatcttcaaatacaagtggacagtcacagtggaggatgggcctactaaaagtgaccccaggtgcctgactaggtactatagttcctttgttaacatggaaagggacctagctagtgggcttataggcccccttctgatctgttataaagaatctgttgatcagaggggcaaccagatcatgtcagacaaaaggaatgtgatactttttagtgtgtttgatgagaataggagctggtacctgactgagaacatccagaggtttttgcccaacccagctggtgtgcagcttgaggacccagagtttcaagcctcaaacatcatgcacagtatcaatggctatgtttttgattctcttcagctgagtgtttgcctgcatgaagtagcttactggtacatcctctcaattggggctcagacagatttcctttcagtgttcttcagtggctacactttcaagcacaaaatggtctatgaggacactctgaccttattcccttttagtggagagacagtctttatgagtatggagaatcctgggctgtggattcttgggtgccacaactctgatttcagaaataggggaatgactgccctgctaaaggtgagtagttgtgacaaaaacacaggggattactatgaggacagctatgaggacatcagtgcctatctgctctcaaagaacaatgcaatagagcctaggtccttctcacagaatcctccagttttgaaacaccaccagagagagataaccagaacaacccttcagtctgaccaggaggagatagattatgatgacactatctcagttgagatgaaaaaggaggactttgatatctatgatgaagatgaaaatcagtctcccaggtccttccagaaaaaaacaaggcattacttcatagcagcagtagagagactgtgggattatgggatgtcatccagtcctcatgtcctgaggaacagagctcagagtggctcagtgcctcaattcaagaaagtggtgttccaggagttcacagatggctccttcacacagcctctctacagaggagagctcaatgaacacttggggctgctggggccctatatcagggcagaggtggaggataatattatggtgacatttaggaaccaagcctcaaggccatactcattttattcttccctgatctcatatgaagaggatcagaggcagggggcagaacctaggaagaattttgtaaagcctaatgagactaagacttacttttggaaggtgcaacaccacatggctcccaccaaagatgaatttgattgcaaggcttgggcctacttctctgatgtggatctggagaaagatgtgcattctggcttgataggtcccctactggtttgtcatacaaataccctgaaccctgcacatggcaggcaggtgactgtgcaggagtttgctctcttcttcactatatttgatgagaccaagtcctggtacttcacagagaatatggaaagaaactgcagggccccatgtaatatccagatggaagaccccacctttaaggaaaattataggtttcatgctattaatggatatataatggacaccctcccaggtctggtcatggcacaggaccagaggataaggtggtatctgctttctatgggctctaatgaaaatattcatagcatccactttagtgggcatgtgttcactgtcaggaaaaaagaggagtataaaatggccctgtataacctgtacccaggagtgtttgagacagtagagatgcttcctagtaaggctggcatttggagagtagagtgcctgattggagaacatctccatgctggaatgagcaccctgttcctggtctatagtaacaagtgtcagaccccactgggcatggcctcaggtcatattagggacttccaaatcactgccagtggccaatatgggcagtgggcccccaaactagccaggttgcactattctgggagtattaatgcctggtctacaaaggaaccattttcctggatcaaagtggatctgctggccccaatgatcatacatggaattaagacccagggagccaggcagaagttttcctccctgtacattagtcagttcatcattatgtacagtttggatgggaaaaagtggcagacatataggggtaacagtacaggaacccttatggtgttctttggtaatgttgacagctcaggaatcaagcacaatattttcaaccccccaatcatagccaggtacatcaggctccaccctactcattatagcatcaggagcaccctgagaatggagcttatgggatgtgacctaaactcctgtagtatgcctctgggaatggaatcaaaggccatctctgatgcccaaattacagcctcatcctactttacaaacatgtttgctacttggtcaccatccaaagctagactgcacctgcaggggaggtcaaatgcctggaggccacaagtcaacaaccccaaagaatggctccaagttgactttcagaagactatgaaagtgactggggtgactactcaaggggtaaagtctcttcttaccagtatgtatgtcaaagagtttttgataagctcctcccaggatggccaccagtggacactgttttttcagaatggcaaggtgaaagtgttccaaggaaaccaggattcttttactccagtggtgaacagcctggaccctcctcttctgaccagatacttgagaatccacccccagtcctgggttcatcagattgctctgaggatggaagtgctgggatgtgaggcccaggacctctactaa

SEQ ID NO: 179 - DNA, 4887 bp, Synthetic construct
  source 1..4887: mol_type = other DNA; note = FVIII v8
atgcagattgaactgtctacctgcttctttctgtgcctcctgaggttctgttttagtgccacaaggagatattacctaggagcagttgagctgtcctgggattacatgcagagtgatctgggggagctgccagttgatgctaggttccctccaagggtgcctaagtccttcccctttaatacctcagtggtgtataagaagaccctttttgtagagttcactgatcacctgttcaatatagctaaacccaggccaccttggatggggttgttgggcccaacaattcaggcagaggtttatgacacagtagtgataaccctcaagaatatggctagtcatcctgtgagcctgcatgcagtaggtgtgtcttactggaaggcttcagagggtgctgagtatgatgatcagacttctcagagagagaaggaagatgacaaagtctttccagggggatcacatacatatgtttggcaggttcttaaagagaatggtcctatggccagtgaccccctgtgtctgacttactcctacctgagccatgtggacctggtgaaggatctgaactctgggctgataggtgcactgctggtgtgtagggaaggtagccttgccaaagagaagacccaaacactccacaagttcatcttgctctttgcagtttttgatgaaggaaaaagctggcatagtgaaaccaaaaactctttgatgcaggatagggatgcagctagtgctagggcctggccaaagatgcacacagtcaatggttatgtgaacagaagtcttccaggtctgataggatgccacaggaaaagtgtgtactggcatgtgataggcatgggaaccaccccagaagtgcactcaatattccttgagggtcataccttccttgtgagaaatcataggcaggcatccttggagatcagtcccatcaccttcttaactgcccaaacccttctgatggaccttggtcaatttctgctgtcctgccatatttcttcccatcagcatgatgggatggaggcctatgtaaaggtggattcttgcccagaggaaccccagctgaggatgaagaacaatgaggaagcagaggattatgatgatgatcttactgatagtgagatggatgtggttaggtttgatgatgacaacagccccagctttatccagattaggtctgttgctaagaagcatccaaagacatgggttcattacatagctgcagaagaagaggactgggattatgcccccctagtccttgccccagatgatagatcctataaatctcagtatctgaacaatggacctcagaggataggcaggaagtataagaaagtgagattcatggcatatacagatgagaccttcaagaccagagaagcaatccaacatgaaagtgggattctgggcccactgctgtatggtgaggtgggtgacaccctgctgataatcttcaagaatcaggccagcaggccttataacatctatccacatgggataacagatgtaaggcctctgtatagtagaaggctgcccaaaggggtgaaacacctgaaagactttcctattcttccaggtgaaatcttcaaatacaagtggacagtcacagtggaggatgggcctactaaaagtgaccccaggtgcctgactaggtactatagttcctttgttaacatggaaagggacctagctagtgggcttataggcccccttctgatctgttataaagaatctgttgatcagaggggcaaccagatcatgtcagacaaaaggaatgtgatactttttagtgtgtttgatgagaataggagctggtacctgactgagaacatccagaggtttttgcccaacccagctggtgtgcagcttgaggacccagagtttcaagcctcaaacatcatgcacagtatcaatggctatgtttttgattctcttcagctgagtgtttgcctgcatgaagtagcttactggtacatcctctcaattggggctcagacagatttcctttcagtgttcttcagtggctacactttcaagcacaaaatggtctatgaggacactctgaccttattcccttttagtggagagacagtctttatgagtatggagaatcctgggctgtggattcttgggtgccacaactctgatttcagaaataggggaatgactgccctgctaaaggtgagtagttgtgacaaaaacacaggggattactatgaggacagctatgaggacatcagtgcctatctgctctcaaagaacaatgcaatagagcctaggtccttctcacagaatagcaggcatccctcaaccaggcaaaaacagtttaatgccaccaccataccagagaatgatattgaaaagactgacccttggtttgctcacaggaccccaatgcccaaaatccaaaatgtgtcttcaagtgacctgttaatgctgttgaggcagtcacccaccccacatgggctgtccctgtcagatcttcaagaggccaaatatgagaccttcagtgatgatccttcaccaggagccatagattctaacaactccctgtcagaaatgacacacttcagacctcagttacatcattctggggatatggtgttcacacctgaatctggtctccagctgaggttaaatgagaaacttgggactacagcagctactgagctgaaaaaacttgattttaaggtgagctcaacaagcaataacctgatttccaccattcctagtgacaacctagctgctggcactgataacacctcttctctgggccctccttcaatgccagtacattatgattcccaacttgataccaccctctttgggaagaagtctagcccacttacagaatcaggtggcccacttagtctctcagaggagaataatgattccaaacttttggagagtgggctgatgaactctcaagagagcagttgggggaagaatgtatccaggtccttccagaaaaaaacaaggcattacttcatagcagcagtagagagactgtgggattatgggatgtcatccagtcctcatgtcctgaggaacagagctcagagtggctcagtgcctcaattcaagaaagtggtgttccaggagttcacagatggctccttcacacagcctctctacagaggagagctcaatgaacacttggggctgctggggccctatatcagggcagaggtggaggataatattatggtgacatttaggaaccaagcctcaaggccatactcattttattcttccctgatctcatatgaagaggatcagaggcagggggcagaacctaggaagaattttgtaaagcctaatgagactaagacttacttttggaaggtgcaacaccacatggctcccaccaaagatgaatttgattgcaaggcttgggcctacttctctgatgtggatctggagaaagatgtgcattctggcttgataggtcccctactggtttgtcatacaaataccctgaaccctgcacatggcaggcaggtgactgtgcaggagtttgctctcttcttcactatatttgatgagaccaagtcctggtacttcacagagaatatggaaagaaactgcagggccccatgtaatatccagatggaagaccccacctttaaggaaaattataggtttcatgctattaatggatatataatggacaccctcccaggtctggtcatggcacaggaccagaggataaggtggtatctgctttctatgggctctaatgaaaatattcatagcatccactttagtgggcatgtgttcactgtcaggaaaaaagaggagtataaaatggccctgtataacctgtacccaggagtgtttgagacagtagagatgcttcctagtaaggctggcatttggagagtagagtgcctgattggagaacatctccatgctggaatgagcaccctgttcctggtctatagtaacaagtgtcagaccccactgggcatggcctcaggtcatattagggacttccaaatcactgccagtggccaatatgggcagtgggcccccaaactagccaggttgcactattctgggagtattaatgcctggtctacaaaggaaccattttcctggatcaaagtggatctgctggccccaatgatcatacatggaattaagacccagggagccaggcagaagttttcctccctgtacattagtcagttcatcattatgtacagtttggatgggaaaaagtggcagacatataggggtaacagtacaggaacccttatggtgttctttggtaatgttgacagctcaggaatcaagcacaatattttcaaccccccaatcatagccaggtacatcaggctccaccctactcattatagcatcaggagcaccctgagaatggagcttatgggatgtgacctaaactcctgtagtatgcctctgggaatggaatcaaaggccatctctgatgcccaaattacagcctcatcctactttacaaacatgtttgctacttggtcaccatccaaagctagactgcacctgcaggggaggtcaaatgcctggaggccacaagtcaacaaccccaaagaatggctccaagttgactttcagaagactatgaaagtgactggggtgactactcaaggggtaaagtctcttcttaccagtatgtatgtcaaagagtttttgataagctcctcccaggatggccaccagtggacactgttttttcagaatggcaaggtgaaagtgttccaaggaaaccaggattcttttactccagtggtgaacagcctggaccctcctcttctgaccagatacttgagaatccacccccagtcctgggttcatcagattgctctgaggatggaagtgctgggatgtgaggcccaggacctctactaa

SEQ ID NO: 180 - DNA, 5013 bp, Synthetic construct
  source 1..5013: mol_type = other DNA; note = FVIII v9
atgcagattgaactgtctacctgcttctttctgtgcctcctgaggttctgttttagtgccacaaggagatattacctaggagcagttgagctgtcctgggattacatgcagagtgatctgggggagctgccagttgatgctaggttccctccaagggtgcctaagtccttcccctttaatacctcagtggtgtataagaagaccctttttgtagagttcactgatcacctgttcaatatagctaaacccaggccaccttggatggggttgttgggcccaacaattcaggcagaggtttatgacacagtagtgataaccctcaagaatatggctagtcatcctgtgagcctgcatgcagtaggtgtgtcttactggaaggcttcagagggtgctgagtatgatgatcagacttctcagagagagaaggaagatgacaaagtctttccagggggatcacatacatatgtttggcaggttcttaaagagaatggtcctatggccagtgaccccctgtgtctgacttactcctacctgagccatgtggacctggtgaaggatctgaactctgggctgataggtgcactgctggtgtgtagggaaggtagccttgccaaagagaagacccaaacactccacaagttcatcttgctctttgcagtttttgatgaaggaaaaagctggcatagtgaaaccaaaaactctttgatgcaggatagggatgcagctagtgctagggcctggccaaagatgcacacagtcaatggttatgtgaacagaagtcttccaggtctgataggatgccacaggaaaagtgtgtactggcatgtgataggcatgggaaccaccccagaagtgcactcaatattccttgagggtcataccttccttgtgagaaatcataggcaggcatccttggagatcagtcccatcaccttcttaactgcccaaacccttctgatggaccttggtcaatttctgctgtcctgccatatttcttcccatcagcatgatgggatggaggcctatgtaaaggtggattcttgcccagaggaaccccagctgaggatgaagaacaatgaggaagcagaggattatgatgatgatcttactgatagtgagatggatgtggttaggtttgatgatgacaacagccccagctttatccagattaggtctgttgctaagaagcatccaaagacatgggttcattacatagctgcagaagaagaggactgggattatgcccccctagtccttgccccagatgatagatcctataaatctcagtatctgaacaatggacctcagaggataggcaggaagtataagaaagtgagattcatggcatatacagatgagaccttcaagaccagagaagcaatccaacatgaaagtgggattctgggcccactgctgtatggtgaggtgggtgacaccctgctgataatcttcaagaatcaggccagcaggccttataacatctatccacatgggataacagatgtaaggcctctgtatagtagaaggctgcccaaaggggtgaaacacctgaaagactttcctattcttccaggtgaaatcttcaaatacaagtggacagtcacagtggaggatgggcctactaaaagtgaccccaggtgcctgactaggtactatagttcctttgttaacatggaaagggacctagctagtgggcttataggcccccttctgatctgttataaagaatctgttgatcagaggggcaaccagatcatgtcagacaaaaggaatgtgatactttttagtgtgtttgatgagaataggagctggtacctgactgagaacatccagaggtttttgcccaacccagctggtgtgcagcttgaggacccagagtttcaagcctcaaacatcatgcacagtatcaatggctatgtttttgattctcttcagctgagtgtttgcctgcatgaagtagcttactggtacatcctctcaattggggctcagacagatttcctttcagtgttcttcagtggctacactttcaagcacaaaatggtctatgaggacactctgaccttattcccttttagtggagagacagtctttatgagtatggagaatcctgggctgtggattcttgggtgccacaactctgatttcagaaataggggaatgactgccctgctaaaggtgagtagttgtgacaaaaacacaggggattactatgaggacagctatgaggacatcagtgcctatctgctctcaaagaacaatgcaatagagcctaggtccttctcacagaatagcaggcatccctcaaccaggcaaaaacagtttaatgccaccaccataccagagaatgatattgaaaagactgacccttggtttgctcacaggaccccaatgcccaaaatccaaaatgtgtcttcaagtgacctgttaatgctgttgaggcagtcacccaccccacatgggctgtccctgtcagatcttcaagaggccaaatatgagaccttcagtgatgatccttcaccaggagccatagattctaacaactccctgtcagaaatgacacacttcagacctcagttacatcattctggggatatggtgttcacacctgaatctggtctccagctgaggttaaatgagaaacttgggactacagcagctactgagctgaaaaaacttgattttaaggtgagctcaacaagcaataacctgatttccaccattcctagtgacaacctagctgctggcactgataacacctcttctctgggccctccttcaatgccagtacattatgattcccaacttgataccaccctctttgggaagaagtctagcccacttacagaatcaggtggcccacttagtctctcagaggagaataatgattccaaacttttggagagtgggctgatgaactctcaagagagcagttgggggaagaatgtatcccagagagagataaccagaacaacccttcagtctgaccaggaggagatagattatgatgacactatctcagttgagatgaaaaaggaggactttgatatctatgatgaagatgaaaatcagtctcccaggtccttccagaaaaaaacaaggcattacttcatagcagcagtagagagactgtgggattatgggatgtcatccagtcctcatgtcctgaggaacagagctcagagtggctcagtgcctcaattcaagaaagtggtgttccaggagttcacagatggctccttcacacagcctctctacagaggagagctcaatgaacacttggggctgctggggccctatatcagggcagaggtggaggataatattatggtgacatttaggaaccaagcctcaaggccatactcattttattcttccctgatctcatatgaagaggatcagaggcagggggcagaacctaggaagaattttgtaaagcctaatgagactaagacttacttttggaaggtgcaacaccacatggctcccaccaaagatgaatttgattgcaaggcttgggcctacttctctgatgtggatctggagaaagatgtgcattctggcttgataggtcccctactggtttgtcatacaaataccctgaaccctgcacatggcaggcaggtgactgtgcaggagtttgctctcttcttcactatatttgatgagaccaagtcctggtacttcacagagaatatggaaagaaactgcagggccccatgtaatatccagatggaagaccccacctttaaggaaaattataggtttcatgctattaatggatatataatggacaccctcccaggtctggtcatggcacaggaccagaggataaggtggtatctgctttctatgggctctaatgaaaatattcatagcatccactttagtgggcatgtgttcactgtcaggaaaaaagaggagtataaaatggccctgtataacctgtacccaggagtgtttgagacagtagagatgcttcctagtaaggctggcatttggagagtagagtgcctgattggagaacatctccatgctggaatgagcaccctgttcctggtctatagtaacaagtgtcagaccccactgggcatggcctcaggtcatattagggacttccaaatcactgccagtggccaatatgggcagtgggcccccaaactagccaggttgcactattctgggagtattaatgcctggtctacaaaggaaccattttcctggatcaaagtggatctgctggccccaatgatcatacatggaattaagacccagggagccaggcagaagttttcctccctgtacattagtcagttcatcattatgtacagtttggatgggaaaaagtggcagacatataggggtaacagtacaggaacccttatggtgttctttggtaatgttgacagctcaggaatcaagcacaatattttcaaccccccaatcatagccaggtacatcaggctccaccctactcattatagcatcaggagcaccctgagaatggagcttatgggatgtgacctaaactcctgtagtatgcctctgggaatggaatcaaaggccatctctgatgcccaaattacagcctcatcctactttacaaacatgtttgctacttggtcaccatccaaagctagactgcacctgcaggggaggtcaaatgcctggaggccacaagtcaacaaccccaaagaatggctccaagttgactttcagaagactatgaaagtgactggggtgactactcaaggggtaaagtctcttcttaccagtatgtatgtcaaagagtttttgataagctcctcccaggatggccaccagtggacactgttttttcagaatggcaaggtgaaagtgttccaaggaaaccaggattcttttactccagtggtgaacagcctggaccccccccttctgaccagatacttgagaatccacccccagtcctgggttcatcagattgctctgaggatggaagtgctgggatgtgaggcccaggacctctactaa

SEQ ID NO: 181 - DNA, 5013 bp, Synthetic construct
  source 1..5013: mol_type = other DNA; note = FVIII v10 (truncated CpG free, GATC minimized)
atgcagattgaactgtctacctgcttctttctgtgcctcctgaggttctgttttagtgccacaaggagatattacttaggggcagttgagctgtcctgggattacatgcagagtgatttaggggagctgccagttgatgctaggttccctccaagggtgcctaagtccttcccctttaatacctcagtggtgtataagaagaccctttttgtagagttcactgaccacctgttcaatatagctaaacccaggccaccttggatggggttgttagggccaacaattcaggcagaggtttatgacacagtagtgataaccctcaagaatatggctagtcaccctgtgagcctgcatgcagtaggtgtgtcttactggaaggcttcagagggtgctgagtatgatgaccagacttctcagagagagaaggaagatgacaaagtctttccagggggaagtcatacatatgtttggcaggttcttaaagagaatggtcctatggccagtgaccccctgtgtctgacttactcctacctgagccatgtggacctggtgaaggatttaaactctgggctgataggtgcactgctggtgtgtagggaaggtagccttgccaaagagaagacccaaacactccacaagttcatattgctctttgcagtttttgatgaaggaaaaagctggcatagtgaaaccaaaaactctttaatgcaggatagggatgcagctagtgctagggcctggccaaaaatgcacacagtcaatggttatgtgaacagaagtcttccaggtctgataggctgccacaggaaaagtgtgtactggcatgtgataggcatgggaaccaccccagaagtgcactcaatattccttgagggtcataccttccttgtgagaaatcataggcaggcaagtttggagataagtcccataaccttcttaactgcccaaacccttctcatggacttagggcaatttctgctgtcctgccatatctcttcccaccagcatgatggcatggaggcctatgtaaaggtggattcttgcccagaggaaccccagctgagaatgaagaacaatgaggaagcagaggattatgatgatgacttaactgatagtgaaatggatgtggttaggtttgatgatgacaacagccccagctttatccagattaggtctgttgctaagaagcacccaaagacatgggttcattacatagctgcagaagaagaggactgggattatgccccccttgtccttgccccagatgatagaagttacaaatctcagtatctgaacaatggacctcagaggataggcaggaagtataagaaagtgagattcatggcatatacagatgagaccttcaagaccagagaagcaatccaacatgaaagtgggattttagggccactgctgtatggtgaggtgggtgacaccctgctgataatcttcaagaatcaggccagcaggccttataacatatatccacatgggataacagatgtaaggcctctgtatagtagaaggctgcccaaaggggtgaaacacctgaaagactttcctattcttccaggtgaaatcttcaaatacaagtggacagtcacagtggaggatgggcctactaaaagtgaccccaggtgcctgactaggtactatagttcctttgttaacatggaaagggacctagctagtgggcttataggcccccttctgatatgttacaaagaatctgttgaccagaggggcaaccagataatgtcagacaaaaggaatgtgatactttttagtgtgtttgatgagaataggagctggtacctgactgagaacatacagaggtttttgcccaacccagctggtgtgcagcttgaggacccagagtttcaagcctcaaacataatgcacagtatcaatggctatgtttttgattctcttcagctgagtgtttgcctgcatgaagtagcttactggtacatactctcaattggggctcagacagatttcctttcagtgttcttcagtggctacactttcaagcacaaaatggtctatgaggacactctgaccttattcccttttagtggagagacagtctttatgagtatggagaatcctgggctgtggattttagggtgccacaactctgatttcagaaataggggaatgactgccctgctaaaggtgagtagttgtgacaaaaacacaggggattactatgaggacagctatgaggacataagtgcctatctgctctcaaagaacaatgcaatagagcctaggtccttctcacagaatagcaggcacccctcaaccaggcaaaaacagtttaatgccaccaccataccagagaatgatattgaaaagactgacccttggtttgctcacaggaccccaatgcccaaaatccaaaatgtgtcttcaagtgacctgttaatgctgttgaggcagtcacccaccccacatgggctgtccctgtcagatttacaagaggccaagtatgagaccttcagtgatgacccttcaccaggagccatagattctaacaactccctgtcagaaatgacacacttcagacctcagttacaccattctggggatatggtgttcacacctgaatctggtctccagctgaggttaaatgagaaattagggactacagcagctactgagctgaagaaacttgattttaaggtgagctcaacaagcaataacctgatttccaccattcctagtgacaacctagctgctggcactgataacacctcttctttagggcctccttcaatgccagtacattatgattcccaacttgataccaccctctttgggaagaagtctagcccacttacagaatcaggtggcccacttagtctctcagaggagaataatgattccaaacttttggagagtgggctcatgaactctcaagagagcagttgggggaagaatgtatcccagagagagataaccagaacaacccttcagtctgaccaggaggagatagattatgatgacactatctcagttgaaatgaaaaaggaggactttgatatctatgatgaggatgaaaatcagtctcccaggtccttccagaagaaaacaaggcattacttcatagcagcagtagagagactgtgggattatggcatgtcaagtagtcctcatgtcctgaggaacagagctcagagtggctcagtgcctcaattcaagaaagtggtgttccaggagttcacagatggctccttcacacagcctctctacagaggagagctcaatgaacacttagggctgttagggccctatattagggcagaggtggaggataatattatggtgacatttaggaaccaagcctcaaggccatactcattctattcttccctgatatcatatgaagaggaccagaggcagggggcagaacctaggaagaattttgtaaagcctaatgagactaagacttacttttggaaggtgcaacaccacatggctcccaccaaagatgaatttgattgcaaggcttgggcctacttctctgatgtggatctggagaaagatgtgcattctggcttgataggtcccctactggtttgtcatacaaataccctgaaccctgcacatggcaggcaggtgactgtgcaggagtttgctctcttcttcactatctttgatgagaccaagtcctggtacttcacagagaatatggaaagaaactgcagggccccatgtaatatccaaatggaagaccccacctttaaggaaaattataggtttcatgctattaatggatatataatggacaccctcccaggtctggtcatggcacaggaccagaggataaggtggtatctgctttctatgggctctaatgagaatatccatagcatacactttagtgggcatgtgttcactgtcaggaagaaagaggagtacaaaatggccctgtataacctgtacccaggagtgtttgagacagtagaaatgcttcctagtaaggctggcatttggagagtagagtgcctgattggagaacatttacatgctggaatgagcaccctgttcctggtctatagtaacaagtgtcagaccccattagggatggcctcaggtcatattagggacttccaaatcactgccagtggccaatatgggcagtgggcccccaaactagccaggttgcactattctgggagtattaatgcctggtctacaaaggaaccattttcctggataaaagtggatttactggccccaatgataatacatggaattaagacccagggagccaggcagaagttttcctccctgtacattagtcagttcataattatgtacagtttggatgggaaaaagtggcagacatataggggtaacagtacaggaacccttatggtgttctttggtaatgttgacagctcaggaatcaagcacaatatcttcaaccccccaatcatagccaggtacataaggctccaccctactcattatagcataaggagcaccctgagaatggagcttatgggctgtgacctaaactcctgtagtatgcctttagggatggaatcaaaggccatatctgatgcccaaattacagcctcaagttactttacaaacatgtttgctacttggtcaccaagtaaagctagactgcacctgcaggggaggtcaaatgcctggaggccacaagtcaacaaccccaaagaatggctccaagttgactttcagaagactatgaaagtgactggggtgactactcaaggggtaaagtctcttcttaccagtatgtatgtcaaagagtttttgataagctcctcccaggatggccaccagtggacactgttctttcagaatggcaaggtgaaagtgttccaaggaaaccaggattcttttactccagtggtgaacagcctggacccccctcttctgaccagatacttgagaatccacccccagtcctgggttcaccagattgctctgagaatggaagtgttagggtgtgaggcccaggacctctactaa

SEQ ID NO: 182 - skipped

SEQ ID NO: 183 - DNA, 104 bp, Synthetic construct
  source 1..104: mol_type = other DNA; note = HBoV genome (accession number JQ923422) nucleotides 19-122
tcttggaatccaatatgtctgccggctcagtcatgcctgcgctgcgcgcagcgcgctgcgcgcgcgcatgatctaatcgccggcagacatattggattccaaga

SEQ ID NO: 184 - DNA, 109 bp, Synthetic construct
  source 1..109: mol_type = other DNA; note = B19 genome (accession number AY386330) nucleotides 129 to 237
gggttggctctgggccagcttgcttggggttgccttgacactaagacaagcggcgcgccgcttgatcttagtggcacgtcaaccccaagcgctggcccagagccaaccc

SEQ ID NO: 185 - skipped

SEQ ID NO: 186 - DNA, 99 bp, Synthetic construct
  source 1..99: mol_type = other DNA; note = wt_AAV1
aacgggtgagggagagacgcgcgagcgagcgagccaccccggacgcctggtttccaggcgtctgccgtctcgagacgagacggccggggtggctcgctc

SEQ ID NO: 187 - DNA, 99 bp, Synthetic construct
  source 1..99: mol_type = other DNA; note = AAV1_Nb.BbvCI_BL
aacgggtgagggggagtcgcgcgagcgagcgagccaccccggacgcctggtttccaggcgtctgccgtctcgagacgagacggccggggtggctcgctc

SEQ ID NO: 188 - DNA, 99 bp, Synthetic construct
  source 1..99: mol_type = other DNA; note = AAV1_Nb.BbvCI_TL
aacgggtgagggcgactcccgcgagcgagcgagccaccccggacgcctggtttccaggcgtctgccgtctcgagacgagacggccggggtggctcgctc

SEQ ID NO: 189 - DNA, 99 bp, Synthetic construct
  source 1..99: mol_type = other DNA; note = AAV1_Nb.BsmI_BL
aacgggtgagggacttacgcgcgagcgagcgagccaccccggacgcctggtttccaggcgtctgccgtctcgagacgagacggccggggtggctcgctc

SEQ ID NO: 190 - DNA, 99 bp, Synthetic construct
  source 1..99: mol_type = other DNA; note = AAV1_Nb.BsmI_TL
aacgggtgagggagagacgcgtaagcgagcgagccaccccggacgcctggtttccaggcgtctgccgtctcgagacgagacggccggggtggctcgctc

SEQ ID NO: 191 - DNA, 99 bp, Synthetic construct
  source 1..99: mol_type = other DNA; note = AAV1_Nb.BsrDI_BL
aacgggtgagggcgttacgcgcgagcgagcgagccaccccggacgcctggtttccaggcgtctgccgtctcgagacgagacggccggggtggctcgctc

SEQ ID NO: 192 - DNA, 99 bp, Synthetic construct
  source 1..99: mol_type = other DNA; note = AAV1_Nb.BsrDI_TL
aacgggtgagggagtaacgcgcgagcgagcgagccaccccggacgcctggtttccaggcgtctgccgtctcgagacgagacggccggggtggctcgctc

SEQ ID NO: 193 - DNA, 99 bp, Synthetic construct
  source 1..99: mol_type = other DNA; note = AAV1_Nb.BssSI_BL
aacgggtgctcgagagacgcgcgagcgagcgagccaccccggacgcctggtttccaggcgtctgccgtctcgagacgagacggccggggtggctcgctc

SEQ ID NO: 194 - DNA, 99 bp, Synthetic construct
  source 1..99: mol_type = other DNA; note = AAV1_Nb.BssSI_TL
aacgggtgagggagcaccgcgcgagcgagcgagccaccccggacgcctggtttccaggcgtctgccgtctcgagacgagacggccggggtggctcgctc

SEQ ID NO: 195 - DNA, 99 bp, Synthetic construct
  source 1..99: mol_type = other DNA; note = AAV1_Nb.BtsI_BL
aacgggtgagggcgtcacgcgcgagcgagcgagccaccccggacgcctggtttccaggcgtctgccgtctcgagacgagacggccggggtggctcgctc

SEQ ID NO: 196 - DNA, 99 bp, Synthetic construct
  source 1..99: mol_type = other DNA; note = AAV1_Nb.BtsI_TL
aacgggtgagggagtgacgcgcgagcgagcgagccaccccggacgcctggtttccaggcgtctgccgtctcgagacgagacggccggggtggctcgctc

SEQ ID NO: 197 - DNA, 99 bp, Synthetic construct
  source 1..99: mol_type = other DNA; note = AAV1_Nt.AlwI_BL
aacgggtgagggagctaggcgcgagcgagcgagccaccccggacgcctggtttccaggcgtctgccgtctcgagacgagacggccggggtggctcgctc

SEQ ID NO: 198 - DNA, 99 bp, Synthetic construct
  source 1..99: mol_type = other DNA; note = AAV1_Nt.AlwI_BL
aacgggtgacctagagacgcgcgagcgagcgagccaccccggacgcctggtttccaggcgtctgccgtctcgagacgagacggccggggtggctcgctc

SEQ ID NO: 199 - DNA, 99 bp, Synthetic construct; identical to SEQ ID NO: 188
  source 1..99: mol_type = other DNA; note = AAV1_Nt.BbvCI_TL

SEQ ID NO: 200 - DNA, 99 bp, Synthetic construct; identical to SEQ ID NO: 187
  source 1..99: mol_type = other DNA; note = AAV1_Nt.BbvCI_BL

SEQ ID NO: 201 - DNA, 99 bp, Synthetic construct
  source 1..99: mol_type = other DNA; note = AAV1_Nt.BsmAI_TL
aacgggtgactctgagacgcgcgagcgagcgagccaccccggacgcctggtttccaggcgtctgccgtctcgagacgagacggccggggtggctcgctc

SEQ ID NO: 202 - DNA, 99 bp, Synthetic construct
  source 1..99: mol_type = other DNA; note = AAV1_Nt.BsmAI_BL
aacgggtgaggcagagacgcgcgagcgagcgagccaccccggacgcctggtttccaggcgtctgccgtctcgagacgagacggccggggtggctcgctc

SEQ ID NO: 203 - DNA, 99 bp, Synthetic construct
  source 1..99: mol_type = other DNA; note = AAV1_Nt.BspQI_TL
aacgggtgagggcttctcgcgcgagcgagcgagccaccccggacgcctggtttccaggcgtctgccgtctcgagacgagacggccggggtggctcgctc

SEQ ID NO: 204 - DNA, 99 bp, Synthetic construct
  source 1..99: mol_type = other DNA; note = AAV1_Nt.BspQI_BL
aacgggtgagggcgagaagcgcgagcgagcgagccaccccggacgcctggtttccaggcgtctgccgtctcgagacgagacggccggggtggctcgctc

SEQ ID NO: 205 - DNA, 100 bp, Synthetic construct
  source 1..100: mol_type = other DNA; note = AAV1_Nt.BstNBI_TL
aacgggtgagggagagacgcgctgagcgagcgagccaccccggacgcctggtttccaggcgtctgccgtctcgagacgagacggccggggtggctcgctc

SEQ ID NO: 206 - DNA, 99 bp, Synthetic construct
  source 1..99: mol_type = other DNA; note = AAV1_Nt.BstNBI_BL
aacggctcagggagagacgcgcgagcgagcgagccaccccggacgcctggtttccaggcgtctgccgtctcgagacgagacggccggggtggctcgctc

SEQ ID NO: 207 - DNA, 99 bp, Synthetic construct
  source 1..99: mol_type = other DNA; note = wt_AAV2
aaccggtgagggagagacgcgcgagcgagcgagtgactccggcccgctggtttccagcgggctgcgggcccgaaacgggcccgccggagtcactcgctc

SEQ ID NO: 208 - DNA, 99 bp, Synthetic construct
  source 1..99: mol_type = other DNA; note = AAV2_Nb.BbvCI_BL
aaccggtgagggggagtcgcgcgagcgagcgagtgactccggcccgctggtttccagcgggctgcgggcccgaaacgggcccgccggagtcactcgctc

SEQ ID NO: 209 - DNA, 99 bp, Synthetic construct
  source 1..99: mol_type = other DNA; note = AAV2_Nb.BbvCI_TL
aaccggtgagggcgactcccgcgagcgagcgagtgactccggcccgctggtttccagcgggctgcgggcccgaaacgggcccgccggagtcactcgctc

SEQ ID NO: 210 - DNA, 99 bp, Synthetic construct
  source 1..99: mol_type = other DNA; note = AAV2_Nb.BsmI_BL
aaccggtgagggacttacgcgcgagcgagcgagtgactccggcccgctggtttccagcgggctgcgggcccgaaacgggcccgccggagtcactcgctc

SEQ ID NO: 211 - DNA, 99 bp, Synthetic construct
  source 1..99: mol_type = other DNA; note = AAV2_Nb.BsmI_TL
aaccggtgagggagagacgcgtaagcgagcgagtgactccggcccgctggtttccagcgggctgcgggcccgaaacgggcccgccggagtcactcgctc

SEQ ID NO: 212 - DNA, 99 bp, Synthetic construct
  source 1..99: mol_type = other DNA; note = AAV2_Nb.BsrDI_BL
aaccggtgagggcgttacgcgcgagcgagcgagtgactccggcccgctggtttccagcgggctgcgggcccgaaacgggcccgccggagtcactcgctc

SEQ ID NO: 213 - DNA, 99 bp, Synthetic construct
  source 1..99: mol_type = other DNA; note = AAV2_Nb.BsrDI_TL
aaccggtgagggagtaacgcgcgagcgagcgagtgactccggcccgctggtttccagcgggctgcgggcccgaaacgggcccgccggagtcactcgctc

SEQ ID NO: 214 - DNA, 99 bp, Synthetic construct
  source 1..99: mol_type = other DNA; note = AAV2_Nb.BssSI_BL
aaccggtgctcgagagacgcgcgagcgagcgagtgactccggcccgctggtttccagcgggctgcgggcccgaaacgggcccgccggagtcactcgctc

SEQ ID NO: 215 - DNA, 99 bp, Synthetic construct
  source 1..99: mol_type = other DNA; note = AAV2_Nb.BssSI_TL
aaccggtgagggagcaccgcgcgagcgagcgagtgactccggcccgctggtttccagcgggctgcgggcccgaaacgggcccgccggagtcactcgctc

SEQ ID NO: 216 - DNA, 99 bp, Synthetic construct
  source 1..99: mol_type = other DNA; note = AAV2_Nb.BtsI_BL
aaccggtgagggcgtcacgcgcgagcgagcgagtgactccggcccgctggtttccagcgggctgcgggcccgaaacgggcccgccggagtcactcgctc

SEQ ID NO: 217 - DNA, 99 bp, Synthetic construct
  source 1..99: mol_type = other DNA; note = AAV2_Nb.BtsI_TL
aaccggtgagggagtgacgcgcgagcgagcgagtgactccggcccgctggtttccagcgggctgcgggcccgaaacgggcccgccggagtcactcgctc

SEQ ID NO: 218 - DNA, 99 bp, Synthetic construct
  source 1..99: mol_type = other DNA; note = AAV2_Nt.AlwI_BL
aaccggtgagggagctaggcgcgagcgagcgagtgactccggcccgctggtttccagcgggctgcgggcccgaaacgggcccgccggagtcactcgctc

SEQ ID NO: 219 - DNA, 99 bp, Synthetic construct
  source 1..99: mol_type = other DNA; note = AAV2_Nt.AlwI_BL
aaccggtgacctagagacgcgcgagcgagcgagtgactccggcccgctggtttccagcgggctgcgggcccgaaacgggcccgccggagtcactcgctc

SEQ ID NO: 220 - DNA, 99 bp, Synthetic construct; identical to SEQ ID NO: 209
  source 1..99: mol_type = other DNA; note = AAV2_Nt.BbvCI_TL

SEQ ID NO: 221 - DNA, 99 bp, Synthetic construct; identical to SEQ ID NO: 208
  source 1..99: mol_type = other DNA; note = AAV2_Nt.BbvCI_BL

SEQ ID NO: 222 - DNA, 99 bp, Synthetic construct
  source 1..99: mol_type = other DNA; note = AAV2_Nt.BsmAI_TL
aaccggtgactctgagacgcgcgagcgagcgagtgactccggcccgctggtttccagcgggctgcgggcccgaaacgggcccgccggagtcactcgctc

SEQ ID NO: 223 - DNA, 99 bp, Synthetic construct
  source 1..99: mol_type = other DNA; note = AAV2_Nt.BsmAI_BL
aaccggtgaggcagagacgcgcgagcgagcgagtgactccggcccgctggtttccagcgggctgcgggcccgaaacgggcccgccggagtcactcgctc

SEQ ID NO: 224 - DNA, 99 bp, Synthetic construct
  source 1..99: mol_type = other DNA; note = AAV2_Nt.BspQI_TL
aaccggtgagggcttctcgcgcgagcgagcgagtgactccggcccgctggtttccagcgggctgcgggcccgaaacgggcccgccggagtcactcgctc

SEQ ID NO: 225 - DNA, 99 bp, Synthetic construct
  source 1..99: mol_type = other DNA; note = AAV2_Nt.BspQI_BL
aaccggtgagggcgagaagcgcgagcgagcgagtgactccggcccgctggtttccagcgggctgcgggcccgaaacgggcccgccggagtcactcgctc

SEQ ID NO: 226 - DNA, 100 bp, Synthetic construct
  source 1..100: mol_type = other DNA; note = AAV2_Nt.BstNBI_TL
aaccggtgagggagagacgcgctgagcgagcgagtgactccggcccgctggtttccagcgggctgcgggcccgaaacgggcccgccggagtcactcgctc

SEQ ID NO: 227 - DNA, 99 bp, Synthetic construct
  source 1..99: mol_type = other DNA; note = AAV2_Nt.BstNBI_BL
aaccgctcagggagagacgcgcgagcgagcgagtgactccggcccgctggtttccagcgggctgcgggcccgaaacgggcccgccggagtcactcgctc

SEQ ID NO: 228 - DNA, 99 bp, Synthetic construct
  source 1..99: mol_type = other DNA; note = wt_AAV3
aaccggtgagggagatacgcgtgagcgagcgagccaccccggaccgctggtttccagcggtctgcctgcacgaaacgtgcaggccggggtggctcgctc

SEQ ID NO: 229 - DNA, 99 bp, Synthetic construct
  source 1..99: mol_type = other DNA; note = AAV3_Nb.BbvCI_BL
aaccggtgagggggagtcgcgtgagcgagcgagccaccccggaccgctggtttccagcggtctgcctgcacgaaacgtgcaggccggggtggctcgctc

SEQ ID NO: 230 - DNA, 99 bp, Synthetic construct
  source 1..99: mol_type = other DNA; note = AAV3_Nb.BbvCI_TL
aaccggtgagggcgactcccgtgagcgagcgagccaccccggaccgctggtttccagcggtctgcctgcacgaaacgtgcaggccggggtggctcgctc

SEQ ID NO: 231 - DNA, 99 bp, Synthetic construct
  source 1..99: mol_type = other DNA; note = AAV3_Nb.BsmI_BL
aaccggtgagggacttacgcgtgagcgagcgagccaccccggaccgctggtttccagcggtctgcctgcacgaaacgtgcaggccggggtggctcgctc

SEQ ID NO: 232 - DNA, 99 bp, Synthetic construct
  source 1..99: mol_type = other DNA; note = AAV3_Nb.BsmI_TL
aaccggtgagggagatacgcgtaagcgagcgagccaccccggaccgctggtttccagcggtctgcctgcacgaaacgtgcaggccggggtggctcgctc

SEQ ID NO: 233 - DNA, 99 bp, Synthetic construct
  source 1..99: mol_type = other DNA; note = AAV3_Nb.BsrDI_BL
aaccggtgagggcgttacgcgtgagcgagcgagccaccccggaccgctggtttccagcggtctgcctgcacgaaacgtgcaggccggggtggctcgctc

SEQ ID NO: 234 - DNA, 99 bp, Synthetic construct
  source 1..99: mol_type = other DNA; note = AAV3_Nb.BsrDI_TL
aaccggtgagggagtaacgcgtgagcgagcgagccaccccggaccgctggtttccagcggtctgcctgcacgaaacgtgcaggccggggtggctcgctc

SEQ ID NO: 235 - DNA, 99 bp, Synthetic construct
  source 1..99: mol_type = other DNA; note = AAV3_Nb.BssSI_BL
aaccggtgctcgagatacgcgtgagcgagcgagccaccccggaccgctggtttccagcggtctgcctgcacgaaacgtgcaggccggggtggctcgctc

SEQ ID NO: 236 - DNA, 99 bp, Synthetic construct
  source 1..99: mol_type = other DNA; note = AAV3_Nb.BssSI_TL
aaccggtgagggagcaccgcgtgagcgagcgagccaccccggaccgctggtttccagcggtctgcctgcacgaaacgtgcaggccggggtggctcgctc

SEQ ID NO: 237 - DNA, 99 bp, Synthetic construct
  source 1..99: mol_type = other DNA; note = AAV3_Nb.BtsI_BL
aaccggtgagggcgtcacgcgtgagcgagcgagccaccccggaccgctggtttccagcggtctgcctgcacgaaacgtgcaggccggggtggctcgctc

SEQ ID NO: 238 - DNA, 99 bp, Synthetic construct
  source 1..99: mol_type = other DNA; note = AAV3_Nb.BtsI_TL
aaccggtgagggagtgacgcgtgagcgagcgagccaccccggaccgctggtttccagcggtctgcctgcacgaaacgtgcaggccggggtggctcgctc

SEQ ID NO: 239 - DNA, 99 bp, Synthetic construct
  source 1..99: mol_type = other DNA; note = AAV3_Nt.AlwI_BL
aaccggtgagggagctaggcgtgagcgagcgagccaccccggaccgctggtttccagcggtctgcctgcacgaaacgtgcaggccggggtggctcgctc

SEQ ID NO: 240 - DNA, 99 bp, Synthetic construct
  source 1..99: mol_type = other DNA; note = AAV3_Nt.AlwI_BL
aaccggtgacctagatacgcgtgagcgagcgagccaccccggaccgctggtttccagcggtctgcctgcacgaaacgtgcaggccggggtggctcgctc

SEQ ID NO: 241 - DNA, 99 bp, Synthetic construct; identical to SEQ ID NO: 230
  source 1..99: mol_type = other DNA; note = AAV3_Nt.BbvCI_TL

SEQ ID NO: 242 - DNA, 99 bp, Synthetic construct; identical to SEQ ID NO: 229
  source 1..99: mol_type = other DNA; note = AAV3_Nt.BbvCI_BL

SEQ ID NO: 243 - DNA, 99 bp, Synthetic construct
  source 1..99: mol_type = other DNA; note = AAV3_Nt.BsmAI_TL
aaccggtgactctgatacgcgtgagcgagcgagccaccccggaccgctggtttccagcggtctgcctgcacgaaacgtgcaggccggggtggctcgctc

SEQ ID NO: 244 - DNA, 99 bp, Synthetic construct
  source 1..99: mol_type = other DNA; note = AAV3_Nt.BsmAI_BL
aaccggtgaggcagagacgcgtgagcgagcgagccaccccggaccgctggtttccagcggtctgcctgcacgaaacgtgcaggccggggtggctcgctc

SEQ ID NO: 245 - DNA, 99 bp, Synthetic construct
  source 1..99: mol_type = other DNA; note = AAV3_Nt.BspQI_TL
aaccggtgagggcttctcgcgtgagcgagcgagccaccccggaccgctggtttccagcggtctgcctgcacgaaacgtgcaggccggggtggctcgctc

SEQ ID NO: 246 - DNA, 99 bp, Synthetic construct
  source 1..99: mol_type = other DNA; note = AAV3_Nt.BspQI_BL
aaccggtgagggcgagaagcgtgagcgagcgagccaccccggaccgctggtttccagcggtctgcctgcacgaaacgtgcaggccggggtggctcgctc

SEQ ID NO: 247 - DNA, 100 bp, Synthetic construct
  source 1..100: mol_type = other DNA; note = AAV3_Nt.BstNBI_TL
aaccggtgagggagatacgcgctgagcgagcgagccaccccggaccgctggtttccagcggtctgcctgcacgaaacgtgcaggccggggtggctcgctc

SEQ ID NO: 248 - DNA, 99 bp, Synthetic construct
  source 1..99: mol_type = other DNA; note = AAV3_Nt.BstNBI_BL
aaccgctcagggagatacgcgtgagcgagcgagccaccccggaccgctggtttccagcggtctgcctgcacgaaacgtgcaggccggggtggctcgctc

SEQ ID NO: 249 - DNA, 125 bp, Synthetic construct
  source 1..125: mol_type = other DNA; note = wt_AAV4_left
aaccggtgagggagatacgcgcgagcgagtgagtgagccgggacctctggtttccagaggtctgacggccggagaccggccgtcccggctcactcactcgctcgcgcgtatctccctcaccggtt

SEQ ID NO: 250 - DNA, 125 bp, Synthetic construct
  source 1..125: mol_type = other DNA; note = AAV4_left_Nb.BbvCI_BL
aaccggtgagggggagtcgcgcgagcgagtgagtgagccgggacctctggtttccagaggtctgacggccggagaccggccgtcccggctcactcactcgctcgcgcgactccccctcaccggtt

SEQ ID NO: 251 - DNA, 125 bp, Synthetic construct
  source 1..125: mol_type = other DNA; note = AAV4_left_Nb.BbvCI_TL
aaccggtgagggcgactcccgcgagcgagtgagtgagccgggacctctggtttccagaggtctgacggccggagaccggccgtcccggctcactcactcgctcgcgggagtcgccctcaccggtt

SEQ ID NO: 252 - DNA, 125 bp, Synthetic construct
  source 1..125: mol_type = other DNA; note = AAV4_left_Nb.BsmI_BL
aaccggtgagggacttacgcgcgagcgagtgagtgagccgggacctctggtttccagaggtctgacggccggagaccggccgtcccggctcactcactcgctcgcgcgtaagtccctcaccggtt

SEQ ID NO: 253 - DNA, 125 bp, Synthetic construct
  source 1..125: mol_type = other DNA; note = AAV4_left_Nb.BsmI_TL
aaccggtgagggagatacgcgtaagcgagtgagtgagccgggacctctggtttccagaggtctgacggccggagaccggccgtcccggctcactcactcgcttacgcgtatctccctcaccggtt

SEQ ID NO: 254 - DNA, 125 bp, Synthetic construct
  source 1..125: mol_type = other DNA; note = AAV4_left_Nb.BsrDI_BL
aaccggtgagggcgttacgcgcgagcgagtgagtgagccgggacctctggtttccagaggtctgacggccggagaccggccgtcccggctcactcactcgctcgcgcgtaacgccctcaccggtt

SEQ ID NO: 255 - DNA, 125 bp, Synthetic construct
  source 1..125: mol_type = other DNA; note = AAV4_left_Nb.BsrDI_TL
aaccggtgagggagtaacgcgcgagcgagtgagtgagccgggacctctggtttccagaggtctgacggccggagaccggccgtcccggctcactcactcgctcgcgcgttactccctcaccggtt

SEQ ID NO: 256 - DNA, 125 bp, Synthetic construct
  source 1..125: mol_type = other DNA; note = AAV4_left_Nb.BssSI_BL
aaccggtgctcgagatacgcgcgagcgagtgagtgagccgggacctctggtttccagaggtctgacggccggagaccggccgtcccggctcactcactcgctcgcgcgtatctcgagcaccggtt

SEQ ID NO: 257 - DNA, 125 bp, Synthetic construct
  source 1..125: mol_type = other DNA; note = AAV4_left_Nb.BssSI_TL
aaccggtgagggagcaccgcgcgagcgagtgagtgagccgggacctctggtttccagaggtctgacggccggagaccggccgtcccggctcactcactcgctcgcgcggtgctccctcaccggtt

SEQ ID NO: 258 - DNA, 125 bp, Synthetic construct
  source 1..125: mol_type = other DNA; note = AAV4_left_Nb.BtsI_BL
aaccggtgagggcgtcacgcgcgagcgagtgagtgagccgggacctctggtttccagaggtctgacggccggagaccggccgtcccggctcactcactcgctcgcgcgtgacgccctcaccggtt

SEQ ID NO: 259 - DNA, 125 bp, Synthetic construct
  source 1..125: mol_type = other DNA; note = AAV4_left_Nb.BtsI_TL
aaccggtgagggagtgacgcgcgagcgagtgagtgagccgggacctctggtttccagaggtctgacggccggagaccggccgtcccggctcactcactcgctcgcgcgtcactccctcaccggtt

SEQ ID NO: 260 - DNA, 125 bp, Synthetic construct
  source 1..125: mol_type = other DNA; note = AAV4_left_Nt.AlwI_BL
aaccggtgagggagctaggcgcgagcgagtgagtgagccgggacctctggtttccagaggtctgacggccggagaccggccgtcccggctcactcactcgctcgcgcctagctccctcaccggtt

SEQ ID NO: 261 - DNA, 125 bp, Synthetic construct
  source 1..125: mol_type = other DNA; note = AAV4_left_Nt.AlwI_BL
aaccggtgacctagatacgcgcgagcgagtgagtgagccgggacctctggtttccagaggtctgacggccggagaccggccgtcccggctcactcactcgctcgcgcgtatctaggtcaccggtt

SEQ ID NO: 262 - DNA, 125 bp, Synthetic construct; identical to SEQ ID NO: 251
  source 1..125: mol_type = other DNA; note = AAV4_left_Nt.BbvCI_TL

SEQ ID NO: 263 - DNA, 125 bp, Synthetic construct; identical to SEQ ID NO: 250
  source 1..125: mol_type = other DNA; note = AAV4_left_Nt.BbvCI_BL

SEQ ID NO: 264 - DNA, 125 bp, Synthetic construct
  source 1..125: mol_type = other DNA; note = AAV4_left_Nt.BsmAI_TL
aaccggtgactctgatacgcgcgagcgagtgagtgagccgggacctctggtttccagaggtctgacggccggagaccggccgtcccggctcactcactcgctcgcgcgtatcagagtcaccggtt

SEQ ID NO: 265 - DNA, 125 bp, Synthetic construct
  source 1..125: mol_type = other DNA; note = AAV4_left_Nt.BsmAI_BL
aaccggtgaggcagagacgcgcgagcgagtgagtgagccgggacctctggtttccagaggtctgacggccggagaccggccgtcccggctcactcactcgctcgcgcgtctctgcctcaccggtt

SEQ ID NO: 266 - DNA, 125 bp, Synthetic construct
  source 1..125: mol_type = other DNA; note = AAV4_left_Nt.BspQI_TL
aaccggtgagggcttctcgcgcgagcgagtgagtgagccgggacctctggtttccagaggtctgacggccggagaccggccgtcccggctcactcactcgctcgcgcgagaagccctcaccggtt

SEQ ID NO: 267 - DNA, 125 bp, Synthetic construct
  source 1..125: mol_type = other DNA; note = AAV4_left_Nt.BspQI_BL
aaccggtgagggcgagaagcgcgagcgagtgagtgagccgggacctctggtttccagaggtctgacggccggagaccggccgtcccggctcactcactcgctcgcgcttctcgccctcaccggtt

SEQ ID NO: 268 - DNA, 127 bp, Synthetic construct
  source 1..127: mol_type = other DNA; note = AAV4_left_Nt.BstNBI_TL
aaccggtgagggagatacgcgctgagcgagtgagtgagccgggacctctggtttccagaggtctgacggccggagaccggccgtcccggctcactcactcgctcagcgcgtatctccctcaccggtt

SEQ ID NO: 269 - DNA, 125 bp, Synthetic construct
  source 1..125: mol_type = other DNA; note = AAV4_left_Nt.BstNBI_BL
aaccgctcagggagatacgcgcgagcgagtgagtgagccgggacctctggtttccagaggtctgacggccggagaccggccgtcccggctcactcactcgctcgcgcgtatctccctgagcggtt

SEQ ID NO: 270 - DNA, 125 bp, Synthetic construct
  source 1..125: mol_type = other DNA; note = wt_AAV4_Right
aaccggtgtaatcgatacgcgcgagcgagtgagtgagccgggacctctggtttccagaggtctgacggccggagaccggccgtcccggctcactcactcgctcgcgcgtatctccctcaccggtt

SEQ ID NO: 271 - DNA, 125 bp, Synthetic construct
  source 1..125: mol_type = other DNA; note = AAV4_Right_Nb.BbvCI_BL
aaccggtgtaatggagtcgcgcgagcgagtgagtgagccgggacctctggtttccagaggtctgacggccggagaccggccgtcccggctcactcactcgctcgcgcgactccccctcaccggtt

SEQ ID NO: 272 - DNA, 125 bp, Synthetic construct
  source 1..125: mol_type = other DNA; note = AAV4_Right_Nb.BbvCI_TL
aaccggtgtaatcgactcccgcgagcgagtgagtgagccgggacctctggtttccagaggtctgacggccggagaccggccgtcccggctcactcactcgctcgcgggagtcgccctcaccggtt

SEQ ID NO: 273 - DNA, 125 bp, Synthetic construct
  source 1..125: mol_type = other DNA; note = AAV4_Right_Nb.BsmI_BL
aaccggtgtaatccttacgcgcgagcgagtgagtgagccgggacctctggtttccagaggtctgacggccggagaccggccgtcccggctcactcactcgctcgcgcgtaagtccctcaccggtt

SEQ ID NO: 274 - DNA, 125 bp, Synthetic construct
  source 1..125: mol_type = other DNA; note = AAV4_Right_Nb.BsmI_TL
aaccggtgtaatcgatacgcgtaagcgagtgagtgagccgggacctctggtttccagaggtctgacggccggagaccggccgtcccggctcactcactcgcttacgcgtatctccctcaccggtt

SEQ ID NO: 275 - DNA, 125 bp, Synthetic construct
  source 1..125: mol_type = other DNA; note = AAV4_Right_Nb.BsrDI_BL
aaccggtgtaatcgttacgcgcgagcgagtgagtgagccgggacctctggtttccagaggtctgacggccggagaccggccgtcccggctcactcactcgctcgcgcgtaacgccctcaccggtt

SEQ ID NO: 276 - DNA, 125 bp, Synthetic construct
  source 1..125: mol_type = other DNA; note = AAV4_Right_Nb.BsrDI_TL
aaccggtgtaatcgtaacgcgcgagcgagtgagtgagccgggacctctggtttccagaggtctgacggccggagaccggccgtcccggctcactcactcgctcgcgcgttactccctcaccggtt

SEQ ID NO: 277 - DNA, 125 bp, Synthetic construct
  source 1..125: mol_type = other DNA; note = AAV4_Right_Nb.BssSI_BL
aaccggtgctctcgatacgcgcgagcgagtgagtgagccgggacctctggtttccagaggtctgacggccggagaccggccgtcccggctcactcactcgctcgcgcgtatctcgagcaccggtt

SEQ ID NO: 278 - DNA, 125 bp, Synthetic construct
  source 1..125: mol_type = other DNA; note = AAV4_Right_Nb.BssSI_TL
aaccggtgtaagagcaccgcgcgagcgagtgagtgagccgggacctctggtttccagaggtctgacggccggagaccggccgtcccggctcactcactcgctcgcgcggtgctccctcaccggtt

SEQ ID NO: 279 - DNA, 125 bp, Synthetic construct
  source 1..125: mol_type = other DNA; note = AAV4_Right_Nb.BtsI_BL
aaccggtgtaatcgtcacgcgcgagcgagtgagtgagccgggacctctggtttccagaggtctgacggccggagaccggccgtcccggctcactcactcgctcgcgcgtgacgccctcaccggtt

SEQ ID NO: 280 - DNA, 125 bp, Synthetic construct
  source 1..125: mol_type = other DNA; note = AAV4_Right_Nb.BtsI_TL
aaccggtgtaatcgtgacgcgcgagcgagtgagtgagccgggacctctggtttccagaggtctgacggccggagaccggccgtcccggctcactcactcgctcgcgcgtcactccctcaccggtt

SEQ ID NO: 281 - DNA, 125 bp, Synthetic construct
  source 1..125: mol_type = other DNA; note = AAV4_Right_Nt.AlwI_BL
aaccggtgtaatcgctaggcgcgagcgagtgagtgagccgggacctctggtttccagaggtctgacggccggagaccggccgtcccggctcactcactcgctcgcgcctagctccctcaccggtt

SEQ ID NO: 282 - DNA, 125 bp, Synthetic construct
  source 1..125: mol_type = other DNA; note = AAV4_Right_Nt.AlwI_BL
aaccggtgtcctagatacgcgcgagcgagtgagtgagccgggacctctggtttccagaggtctgacggccggagaccggccgtcccggctcactcactcgctcgcgcgtatctaggtcaccggtt

SEQ ID NO: 283 - DNA, 125 bp, Synthetic construct; identical to SEQ ID NO: 272
  source 1..125: mol_type = other DNA; note = AAV4_Right_Nt.BbvCI_TL

SEQ ID NO: 284 - DNA, 125 bp, Synthetic construct; identical to SEQ ID NO: 271
  source 1..125: mol_type = other DNA; note = AAV4_Right_Nt.BbvCI_BL

SEQ ID NO: 285 - DNA, 125 bp, Synthetic construct
  source 1..125: mol_type = other DNA; note = AAV4_Right_Nt.BsmAI_TL
aaccggtgtctctgatacgcgcgagcgagtgagtgagccgggacctctggtttccagaggtctgacggccggagaccggccgtcccggctcactcactcgctcgcgcgtatcagagtcaccggtt

SEQ ID NO: 286 - DNA, 125 bp, Synthetic construct
  source 1..125: mol_type = other DNA; note = AAV4_Right_Nt.BsmAI_BL
aaccggtgtaacagagacgcgcgagcgagtgagtgagccgggacctctggtttccagaggtctgacggccggagaccggccgtcccggctcactcactcgctcgcgcgtctctgcctcaccggtt

SEQ ID NO: 287 - DNA, 125 bp, Synthetic construct
  source 1..125: mol_type = other DNA; note = AAV4_Right_Nt.BspQI_TL
aaccggtgtaatcttctcgcgcgagcgagtgagtgagccgggacctctggtttccagaggtctgacggccggagaccggccgtcccggctcactcactcgctcgcgcgagaagccctcaccggtt

SEQ ID NO: 288 - DNA, 125 bp, Synthetic construct
  source 1..125: mol_type = other DNA; note = AAV4_Right_Nt.BspQI_BL
aaccggtgtaatcgagaagcgcgagcgagtgagtgagccgggacctctggtttccagaggtctgacggccggagaccggccgtcccggctcactcactcgctcgcgcttctcgccctcaccggtt

SEQ ID NO: 289 - DNA, 127 bp, Synthetic construct
  source 1..127: mol_type = other DNA; note = AAV4_Right_Nt.BstNBI_TL
aaccggtgtaatcgatacgcgctgagcgagtgagtgagccgggacctctggtttccagaggtctgacggccggagaccggccgtcccggctcactcactcgctcagcgcgtatctccctcaccggtt

SEQ ID NO: 290 - DNA, 125 bp, Synthetic construct
  source 1..125: mol_type = other DNA; note = AAV4_Right_Nt.BstNBI_BL
aaccggtctcagcgatacgcgcgagcgagtgagtgagccgggacctctggtttccagaggtctgacggccggagaccggccgtcccggctcactcactcgctcgcgcgtatctctgagaccggtt

SEQ ID NO: 291 - DNA, 137 bp, Synthetic construct
  source 1..137: mol_type = other DNA; note = wt_AAV5
gagaggggggacagcgcaagcgagcgagcgaccgagcaaacccccccaccgtcgagtttctcgacggtctgctgccgggagaccggcagcgggggggtttgctcggtcgctcgctcgcttgcgctgtcccccctctc

SEQ ID NO: 292 - DNA, 137 bp, Synthetic construct
  source 1..137: mol_type = other DNA; note = AAV5_Nb.BbvCI_BL
gagaggggagtcggcgcaagcgagcgagcgaccgagcaaacccccccaccgtcgagtttctcgacggtctgctgccgggagaccggcagcgggggggtttgctcggtcgctcgctcgcttgcgccgactcccctctc

SEQ ID NO: 293 - DNA, 137 bp, Synthetic construct
  source 1..137: mol_type = other DNA; note = AAV5_Nb.BbvCI_TL
gagaggggcgactccgcaagcgagcgagcgaccgagcaaacccccccaccgtcgagtttctcgacggtctgctgccgggagaccggcagcgggggggtttgctcggtcgctcgctcgcttgcggagtcgcccctctc

SEQ ID NO: 294 - DNA, 137 bp, Synthetic construct
  source 1..137: mol_type = other DNA; note = AAV5_Nb.BsmI_BL
gagaggggcttacgcgcaagcgagcgagcgaccgagcaaacccccccaccgtcgagtttctcgacggtctgctgccgggagaccggcagcgggggggtttgctcggtcgctcgctcgcttgcgcgtaagcccctctc

SEQ ID NO: 295 - DNA, 137 bp, Synthetic construct
  source 1..137: mol_type = other DNA; note = AAV5_Nb.BsmI_TL
gagaggggggacagcgtaagcgagcgagcgaccgagcaaacccccccaccgtcgagtttctcgacggtctgctgccgggagaccggcagcgggggggtttgctcggtcgctcgctcgcttacgctgtcccccctctc

SEQ ID NO: 296 - DNA, 137 bp, Synthetic construct
  source 1..137: mol_type = other DNA; note = AAV5_Nb.BsrDI_BL
gagaggcgttacagcgcaagcgagcgagcgaccgagcaaacccccccaccgtcgagtttctcgacggtctgctgccgggagaccggcagcgggggggtttgctcggtcgctcgctcgcttgcgctgtaacgcctctc

SEQ ID NO: 297 - DNA, 137 bp, Synthetic construct
  source 1..137: mol_type = other DNA; note = AAV5_Nb.BsrDI_TL
gagagggggtaacgcgcaagcgagcgagcgaccgagcaaacccccccaccgtcgagtttctcgacggtctgctgccgggagaccggcagcgggggggtttgctcggtcgctcgctcgcttgcgcgttaccccctctc

SEQ ID NO: 298 - DNA, 137 bp, Synthetic construct
  source 1..137: mol_type = other DNA; note = AAV5_Nb.BssSI_BL
gagtgctcggacagcgcaagcgagcgagcgaccgagcaaacccccccaccgtcgagtttctcgacggtctgctgccgggagaccggcagcgggggggtttgctcggtcgctcgctcgcttgcgctgtccgagcactc

SEQ ID NO: 299 - DNA, 137 bp, Synthetic construct
  source 1..137: mol_type = other DNA; note = AAV5_Nb.BssSI_TL
gagagggagcacagcgcaagcgagcgagcgaccgagcaaacccccccaccgtcgagtttctcgacggtctgctgccgggagaccggcagcgggggggtttgctcggtcgctcgctcgcttgcgctgtgctccctctc

SEQ ID NO: 300 - DNA, 137 bp, Synthetic construct
  source 1..137: mol_type = other DNA; note = AAV5_Nb.BtsI_BL
gagaggcgtcacagcgcaagcgagcgagcgaccgagcaaacccccccaccgtcgagtttctcgacggtctgctgccgggagaccggcagcgggggggtttgctcggtcgctcgctcgcttgcgctgtgacgcctctc

SEQ ID NO: 301 - DNA, 137 bp, Synthetic construct
  source 1..137: mol_type = other DNA; note = AAV5_Nb.BtsI_TL
gagaggggtgacggcgcaagcgagcgagcgaccgagcaaacccccccaccgtcgagtttctcgacggtctgctgccgggagaccggcagcgggggggtttgctcggtcgctcgctcgcttgcgccgtcacccctctc

SEQ ID NO: 302 - DNA, 137 bp, Synthetic construct
  source 1..137: mol_type = other DNA; note = AAV5_Nt.AlwI_BL
gagaggggggacagcgctagggagcgagcgaccgagcaaacccccccaccgtcgagtttctcgacggtctgctgccgggagaccggcagcgggggggtttgctcggtcgctcgctccctagcgctgtcccccctctc

SEQ ID NO: 303 - DNA, 137 bp, Synthetic construct
  source 1..137: mol_type = other DNA; note = AAV5_Nt.AlwI_BL
gagagggggcctagcgcaagcgagcgagcgaccgagcaaacccccccaccgtcgagtttctcgacggtctgctgccgggagaccggcagcgggggggtttgctcggtcgctcgctcgcttgcgctaggccccctctc

SEQ ID NO: 304 - DNA, 137 bp, Synthetic construct; identical to SEQ ID NO: 293
  source 1..137: mol_type = other DNA; note = AAV5_Nt.BbvCI_TL

SEQ ID NO: 305 - DNA, 137 bp, Synthetic construct; identical to SEQ ID NO: 292
  source 1..137: mol_type = other DNA; note = AAV5_Nt.BbvCI_BL

SEQ ID NO: 306 - DNA, 137 bp, Synthetic construct
  source 1..137: mol_type = other DNA; note = AAV5_Nt.BsmAI_TL
gagagggggctctgcgcaagcgagcgagcgaccgagcaaacccccccaccgtcgagtttctcgacggtctgctgccgggagaccggcagcgggggggtttgctcggtcgctcgctcgcttgcgcagagccccctctc

SEQ ID NO: 307 - DNA, 137 bp, Synthetic construct
  source 1..137: mol_type = other DNA; note = AAV5_Nt.BsmAI_BL
gagagggggcagagcgcaagcgagcgagcgaccgagcaaacccccccaccgtcgagtttctcgacggtctgctgccgggagaccggcagcgggggggtttgctcggtcgctcgctcgcttgcgctctgccccctctc

SEQ ID NO: 308 - DNA, 137 bp, Synthetic construct
  source 1..137: mol_type = other DNA; note = AAV5_Nt.BspQI_TL
gagagggcttctcgcgcaagcgagcgagcgaccgagcaaacccccccaccgtcgagtttctcgacggtctgctgccgggagaccggcagcgggggggtttgctcggtcgctcgctcgcttgcgcgagaagccctctc

SEQ ID NO: 309 - DNA, 137 bp, Synthetic construct
  source 1..137: mol_type = other DNA; note = AAV5_Nt.BspQI_BL
gagagggcgagaagcgcaagcgagcgagcgaccgagcaaacccccccaccgtcgagtttctcgacggtctgctgccgggagaccggcagcgggggggtttgctcggtcgctcgctcgcttgcgcttctcgccctctc

SEQ ID NO: 310 - DNA, 139 bp, Synthetic construct
  source 1..139: mol_type = other DNA; note = AAV5_Nt.BstNBI_TL
gagaggggggacagcgctgagcgagcgagcgaccgagcaaacccccccaccgtcgagtttctcgacggtctgctgccgggagaccggcagcgggggggtttgctcggtcgctcgctcgctcagcgctgtcccccctctc

SEQ ID NO: 311 - DNA, 137 bp, Synthetic construct
  source 1..137: mol_type = other DNA; note = AAV5_Nt.BstNBI_BL
gagagggggctcagcgcaagcgagcgagcgaccgagcaaacccccccaccgtcgagtttctcgacggtctgctgccgggagaccggcagcgggggggtttgctcggtcgctcgctcgcttgcgctgagccccctctc

SEQ ID NO: 312 - DNA, 125 bp, Synthetic construct
  source 1..125: mol_type = other DNA; note = wt_AAV7
aaccggtgagggagatacgcgcgagcgagcgagccaccccggacgcctggtttccaggcgtctgccgtctcgagacgagacggccggggtggctcgctcgctcgcgcgtatctccctcaccggtt

SEQ ID NO: 313 - DNA, 125 bp, Synthetic construct
  source 1..125: mol_type = other DNA; note = AAV7_Nb.BbvCI_BL
aaccggtgagggggagtcgcgcgagcgagcgagccaccccggacgcctggtttccaggcgtctgccgtctcgagacgagacggccggggtggctcgctcgctcgcgcgactccccctcaccggtt

SEQ ID NO: 314 - DNA, 125 bp, Synthetic construct
  source 1..125: mol_type = other DNA; note = AAV7_Nb.BbvCI_TL
aaccggtgagggcgactcccgcgagcgagcgagccaccccggacgcctggtttccaggcgtctgccgtctcgagacgagacggccggggtggctcgctcgctcgcgggagtcgccctcaccggtt

SEQ ID NO: 315 - DNA, 125 bp, Synthetic construct
  source 1..125: mol_type = other DNA; note = AAV7_Nb.BsmI_BL
aaccggtgagggacttacgcgcgagcgagcgagccaccccggacgcctggtttccaggcgtctgccgtctcgagacgagacggccggggtggctcgctcgctcgcgcgtaagtccctcaccggtt

SEQ ID NO: 316 - DNA, 125 bp, Synthetic construct
  source 1..125: mol_type = other DNA; note = AAV7_Nb.BsmI_TL
aaccggtgagggagatacgcgtaagcgagcgagccaccccggacgcctggtttccaggcgtctgccgtctcgagacgagacggccggggtggctcgctcgcttacgcgtatctccctcaccggtt

SEQ ID NO: 317 - DNA, 125 bp, Synthetic construct
  source 1..125: mol_type = other DNA; note = AAV7_Nb.BsrDI_BL
aaccggtgagggcgttacgcgcgagcgagcgagccaccccggacgcctggtttccaggcgtctgccgtctcgagacgagacggccggggtggctcgctcgctcgcgcgtaacgccctcaccggtt

SEQ ID NO: 318 - DNA, 125 bp, Synthetic construct
  source 1..125: mol_type = other DNA; note = AAV7_Nb.BsrDI_TL
aaccggtgagggagtaacgcgcgagcgagcgagccaccccggacgcctggtttccaggcgtctgccgtctcgagacgagacggccggggtggctcgctcgctcgcgcgttactccctcaccggtt

SEQ ID NO: 319 - DNA, 125 bp, Synthetic construct
  source 1..125: mol_type = other DNA; note = AAV7_Nb.BssSI_BL
aaccggtgctcgagatacgcgcgagcgagcgagccaccccggacgcctggtttccaggcgtctgccgtctcgagacgagacggccggggtggctcgctcgctcgcgcgtatctcgagcaccggtt

SEQ ID NO: 320 - DNA, 125 bp, Synthetic construct
  source 1..125: mol_type = other DNA; note = AAV7_Nb.BssSI_TL
aaccggtgagggagcaccgcgcgagcgagcgagccaccccggacgcctggtttccaggcgtctgccgtctcgagacgagacggccggggtggctcgctcgctcgcgcggtgctccctcaccggtt

SEQ ID NO: 321 - DNA, 125 bp, Synthetic construct
  source 1..125: mol_type = other DNA; note = AAV7_Nb.BtsI_BL
aaccggtgagggcgtcacgcgcgagcgagcgagccaccccggacgcctggtttccaggcgtctgccgtctcgagacgagacggccggggtggctcgctcgctcgcgcgtgacgccctcaccggtt

SEQ ID NO: 322 - DNA, 125 bp, Synthetic construct
  source 1..125: mol_type = other DNA; note = AAV7_Nb.BtsI_TL
aaccggtgagggagtgacgcgcgagcgagcgagccaccccggacgcctggtttccaggcgtctgccgtctcgagacgagacggccggggtggctcgctcgctcgcgcgtcactccctcaccggtt

SEQ ID NO: 323 - DNA, 125 bp, Synthetic construct
  source 1..125: mol_type = other DNA; note = AAV7_Nt.AlwI_BL
aaccggtgagggagctaggcgcgagcgagcgagccaccccggacgcctggtttccaggcgtctgccgtctcgagacgagacggccggggtggctcgctcgctcgcgcctagctccctcaccggtt

SEQ ID NO: 324 - DNA, 125 bp, Synthetic construct
  source 1..125: mol_type = other DNA; note = AAV7_Nt.AlwI_BL
aaccggtgacctagatacgcgcgagcgagcgagccaccccggacgcctggtttccaggcgtctgccgtctcgagacgagacggccggggtggctcgctcgctcgcgcgtatctaggtcaccggtt

SEQ ID NO: 325 - DNA, 125 bp, Synthetic construct; identical to SEQ ID NO: 314
  source 1..125: mol_type = other DNA; note = AAV7_Nt.BbvCI_TL

SEQ ID NO: 326 - DNA, 125 bp, Synthetic construct; identical to SEQ ID NO: 313
  source 1..125: mol_type = other DNA; note = AAV7_Nt.BbvCI_BL

SEQ ID NO: 327 - DNA, 125 bp, Synthetic construct
  source 1..125: mol_type = other DNA; note = AAV7_Nt.BsmAI_TL
aaccggtgactctgatacgcgcgagcgagcgagccaccccggacgcctggtttccaggcgtctgccgtctcgagacgagacggccggggtggctcgctcgctcgcgcgtatcagagtcaccggtt

SEQ ID NO: 328 - DNA, 125 bp, Synthetic construct
  source 1..125: mol_type = other DNA; note = AAV7_Nt.BsmAI_BL
aaccggtgaggcagagacgcgcgagcgagcgagccaccccggacgcctggtttccaggcgtctgccgtctcgagacgagacggccggggtggctcgctcgctcgcgcgtctctgcctcaccggtt

SEQ ID NO: 329 - DNA, 125 bp, Synthetic construct
  source 1..125: mol_type = other DNA; note = AAV7_Nt.BspQI_TL
aaccggtgagggcttctcgcgcgagcgagcgagccaccccggacgcctggtttccaggcgtctgccgtctcgagacgagacggccggggtggctcgctcgctcgcgcgagaagccctcaccggtt

SEQ ID NO: 330 - DNA, 125 bp, Synthetic construct
  source 1..125: mol_type = other DNA; note = AAV7_Nt.BspQI_BL
aaccggtgagggcgagaagcgcgagcgagcgagccaccccggacgcctggtttccaggcgtctgccgtctcgagacgagacggccggggtggctcgctcgctcgcgcttctcgccctcaccggtt

SEQ ID NO: 331 - DNA, 127 bp, Synthetic construct
  source 1..127: mol_type = other DNA; note = AAV7_Nt.BstNBI_TL
aaccggtgagggagatacgcgctgagcgagcgagccaccccggacgcctggtttccaggcgtctgccgtctcgagacgagacggccggggtggctcgctcgctcagcgcgtatctccctcaccggtt

SEQ ID NO: 332 - DNA, 125 bp, Synthetic construct
  source 1..125: mol_type = other DNA; note = AAV7_Nt.BstNBI_BL
aaccgctcagggagatacgcgcgagcgagcgagccaccccggacgcctggtttccaggcgtctgccgtctcgagacgagacggccggggtggctcgctcgctcgcgcgtatctccctgagcggtt

SEQ ID NO: 333 - skipped

SEQ ID NO: 334 - DNA, 121 bp, Synthetic construct
  source 1..121: mol_type = other DNA
ggccactcccgaagagcgcgctcgctatctcactgaggccgggcgaccaaaggtcgcccgacgcccgggctttgcccgggcggcctcagtgagatagcgagcgcgctcttcgggagtggcc

SEQ ID NO: 335 - DNA, 1656 bp, Synthetic construct
  source 1..1656: mol_type = other DNA; note = Construct 3
tgcgcgactcgctcgctcactgaggccgggcgaccaaaggtcgcccgacgcccgggctttgcccgggcggcctcagtgagcgagcgagtcgcgcagagaggttaaaaccaactagacaactttgtatatctagagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgattgactgagtgcggccgcggcaggctgccaccatggaaaccgatacactgctgctttgggtacttttgctgtgggtgcccggcagtacgggcgacgccgcacaaccagctaggagagcagtccggagcctggtcccctcttccgaggccgaggctgaacggggaaagctgccaggcaagaaactccccttagaggttctcatcgagctggaagcaaacgcacgcaaggccggttgcactaggggctgcctcatctgcctcagcaagatcaaatgtacagcaaagatgaagaagtatattcctggccgctgtgcagactacggaggcgacaaaaaaacaggacaagctggtatagttggcgccatcgtagatatccccgagattagtgggttcaaggaaatggagccaatggagcaattcattgcacaggttgatagatgtgcggattgcactactggttgccttaagggcttggcgaatgtcaagtgtagtgatcttctgaaaaagtggctacccggccggtgtgccaccttcgctgacaaaatacagagcgaagtcgacaatattaaaggacttgccggggactaatgatagtgacacaaagtgacgcgtcctagagctcgcactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggcgctagcgcaggaaccccttttaatggagttggcgagtccctctctgcgcgctcgctcgctcactgaggccgggcgaccaaaggtcgcccgacgcccgggctttgcccgggcggcctcagtgagcgagcgagcgcgcagagatcgactcctcggccacttggaggggccggggggacgacgcaatctggagtggaaagaacccccgtctatgcggcttaaagcacggccagggaatagtggatcaagtgtactgacatgtgccggagtccctccatgcccagatcgactccctcgag

SEQ ID NO: 336 - DNA, 125 bp, Synthetic construct
  source 1..125: mol_type = other DNA
ttggtcactccctctctgtacactcactcactcactgatccctggataccaaaggtatccagacacccagtctttgactgggtgggatcagtgagtgagtgagtgtacagagagggagtgaccaa

SEQ ID NO: 337 - DNA, 6433 bp, Synthetic construct
  source 1..6433: mol_type = other DNA
gttcgagatcgctcttcagaagagcgcggagtcacattcgcgactcgcaatgcattgcagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatcccagctcggtagccacctgacgtcgctcttcgcgctcgctcgctcactgaggccgggcgaccaaaggtcgcccgacgcccgggctttgcccgggcggcctcagtgagcgagcgagcgcgaagagcgggagtgagtggccaaaaagtacccggccagaaaggaaccgtaaaaccaactagacagccggtgactttgttccccagctcggttcattctcaagcctcagacaggagaaccgggactagtatatgagctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagacacccacacaaaggaaaagggcctttccgtcctcagccgtcgcttcatgtgacaccacggagtaccgggcgccgtccaggcacctcgattagttctcgatcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagcggccgcggcaggctgccaccatggaggatgccaagaatattaagaaaggccctgccccattctaccctctggaagatggcactgctggtgagcaactgcacaaggccatgaagaggtatgccctggtccctggcacaattgccttcactgatgctcacattgaggtggacatcacctatgctgaatactttgagatgtctgtgaggctggcagaagccatgaaaagatatggactgaacaccaaccacaggattgtggtgtgctctgagaactctctccagttcttcatgcctgtgttgggagccctgttcattggagtggctgtggcccctgccaatgacatctacaatgagagagagctcctgaacagcatgggcatcagccagccaactgtggtctttgtgagcaagaagggcctgcaaaagatcctgaatgtgcagaagaagctgcccatcatccagaagatcatcatcatggacagcaagactgactaccagggcttccagagcatgtatacctttgtgaccagccacctcccccctggcttcaatgagtatgactttgtgcctgagagctttgacagggacaagacaattgctctgattatgaacagctctggctccactggactgcccaaaggtgtggctctgccccacagaactgcttgtgtgagattcagccatgccagagaccccatctttggcaaccagatcatccctgacactgccatcctgtctgtggttccattccatcatggctttggcatgttcacaacactggggtacctgatctgtggcttcagagtggtgctgatgtataggtttgaggaggagctgtttctgaggagcttgcaagactacaagatccagtctgccctgctggtgcccactctgttcagcttctttgccaagagcaccctcattgacaagtatgacctgagcaacctgcatgagattgcctctggaggagcacccctgagcaaggaggtgggtgaggctgtggcaaagaggttccatctcccaggaatcagacagggctatggcctgactgagaccacctctgccatcctcatcacccctgaaggagatgacaagcctggtgctgtgggcaaggtggttcccttttttgaggccaaggtggtggacctggacactggcaagaccctgggagtgaaccagaggggtgagctgtgtgtgaggggtcccatgatcatgtctggctatgtgaacaaccctgaggccaccaatgccctgattgacaaggatggctggctgcactctggtgatattgcctactgggatgaggatgagcactttttcattgtggacaggctgaagagtctcatcaagtacaaaggctaccaagtggcacctgctgagcttgagagcatcctgctccagcaccccaacatctttgatgctggtgtggctggcctgcctgatgatgatgctggagagctgcctgctgctgttgtggttctggagcatggaaagaccatgactgagaaggagattgtggactatgtggccagtcaggtgaccactgccaagaagctgaggggaggtgtggtgtttgtggatgaggtgccaaagggtctgactggcaagctggatgccagaaagatcagagagatcctgatcaaggccaagaagggaggaaagattgcagtttaaggatcctgacacaaagtgacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggctttgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgcgtcctagagctcgcactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggcgctagcgcagggggaccccaagtgacgattgaaccccgattcaggtacccattggaaattttaatggagtaacataactgctggagtagatgaagaacctcttggccactccgctcccgaagagcgcactcgctcgctcggtggggcctggcgaccaaaggtcgccagacggacgtgctttgcacgtccggccccaccgagcgagcgagtgcgctcttcgggccacttggaggggccggggggacgacgcaatctggagtggaaagaacccccgtctatgcggcttaaagcacggccagggaatagtggatcaagtgtactgacatgtgccggcaatgcattgcgtcccgagtcacattcgcgactctcgacgctcttcagaagagcagcttagcttcaatagctcaatgatatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaaggggctcttcagaagagcgagtcacattcgcgactctgcaatgcattgccatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagta

SEQ ID NO: 338 - DNA, 6545 bp, Synthetic construct
  source 1..6545: mol_type = other DNA
gttcgagatcgctcttcagaagagcgcggagtcacattcgcgactcgcaatgcattgcagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatcccagctcggtagccacctgacgtcgctcttcggtggttgtacagacgccatcttggaatccaatatgtctgccggctcagtcatgcctgcgctgcgcgcagcgcgctgcgcgcgcgcatgatctaatcgccggcagacatattggattccaagatggcgtctgtacaaccaccgaagagcggagtggccaaaaagtacccggccagaaaggaaccgtaaaaccaactagacagccggtgactttgttccccagctcggttcattctcaagcctcagacaggagaaccgggactagtatatgagctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagacacccacacaaaggaaaagggcctttccgtcctcagccgtcgcttcatgtgacaccacggagtaccgggcgccgtccaggcacctcgattagttctcgatcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagcggccgcggcaggctgccaccatggaggatgccaagaatattaagaaaggccctgccccattctaccctctggaagatggcactgctggtgagcaactgcacaaggccatgaagaggtatgccctggtccctggcacaattgccttcactgatgctcacattgaggtggacatcacctatgctgaatactttgagatgtctgtgaggctggcagaagccatgaaaagatatggactgaacaccaaccacaggattgtggtgtgctctgagaactctctccagttcttcatgcctgtgttgggagccctgttcattggagtggctgtggcccctgccaatgacatctacaatgagagagagctcctgaacagcatgggcatcagccagccaactgtggtctttgtgagcaagaagggcctgcaaaagatcctgaatgtgcagaagaagctgcccatcatccagaagatcatcatcatggacagcaagactgactaccagggcttccagagcatgtatacctttgtgaccagccacctcccccctggcttcaatgagtatgactttgtgcctgagagctttgacagggacaagacaattgctctgattatgaacagctctggctccactggactgcccaaaggtgtggctctgccccacagaactgcttgtgtgagattcagccatgccagagaccccatctttggcaaccagatcatccctgacactgccatcctgtctgtggttccattccatcatggctttggcatgttcacaacactggggtacctgatctgtggcttcagagtggtgctgatgtataggtttgaggaggagctgtttctgaggagcttgcaagactacaagatccagtctgccctgctggtgcccactctgttcagcttctttgccaagagcaccctcattgacaagtatgacctgagcaacctgcatgagattgcctctggaggagcacccctgagcaaggaggtgggtgaggctgtggcaaagaggttccatctcccaggaatcagacagggctatggcctgactgagaccacctctgccatcctcatcacccctgaaggagatgacaagcctggtgctgtgggcaaggtggttcccttttttgaggccaaggtggtggacctggacactggcaagaccctgggagtgaaccagaggggtgagctgtgtgtgaggggtcccatgatcatgtctggctatgtgaacaaccctgaggccaccaatgccctgattgacaaggatggctggctgcactctggtgatattgcctactgggatgaggatgagcactttttcattgtggacaggctgaagagtctcatcaagtacaaaggctaccaagtggcacctgctgagcttgagagcatcctgctccagcaccccaacatctttgatgctggtgtggctggcctgcctgatgatgatgctggagagctgcctgctgctgttgtggttctggagcatggaaagaccatgactgagaaggagattgtggactatgtggccagtcaggtgaccactgccaagaagctgaggggaggtgtggtgtttgtggatgaggtgccaaagggtctgactggcaagctggatgccagaaagatcagagagatcctgatcaaggccaagaagggaggaaagattgcagtttaaggatcctgacacaaagtgacgcgtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggctttgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcacgcgtcctagagctcgcactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggcgctagcgcagggggaccccaagtgacgattgaaccccgattcaggtacccattggaaattttaatggagtaacataactgctggagtagatgaagaacctcttggccactccgttgcttatgcaatcgcgaaactctatatcttgctcttcttaatgtgttgttgttgtacatgcgccatcttagttttatatcagctggcgccttagttatataacatgcatgttatataactaaggcgccagctgatataaaactaagatggcgcatgtacaacaacaacacattaagaagagcgccggggggacgacgcaatctggagtggaaagaacccccgtctatgcggcttaaagcacggccagggaatagtggatcaagtgtactgacatgtgccggcaatgcattgcgtcccgagtcacattcgcgactctcgacgctcttcagaagagcagcttagcttcaatagctcaatgatatcggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaaggggctcttcagaagagcgagtcacattcgcgactctgcaatgcattgccatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagta

SEQ ID NO: 339 - DNA, 101 bp, Synthetic construct
  source 1..101: mol_type = other DNA
tttgagggttagggttagggttagggtccctttagggcaaagccctaaagacaccctaactttgttagggtgggaccctaaccctaaccctaaccctcaaa

SEQ ID NO: 340 - DNA, 1324 bp, Synthetic construct
  source 1..1324: mol_type = other DNA; note = Endogenous F8 Promoter
taacacattcttgtttgtgcagaactgagctcaatgataacatttttatagcaaccctgatcaagtttcttctcataatctcttacactttgaggcccctgcaggggccctcactctccctaataaacattaacctgagtagggtgtttgagctcaccatggctacattctgatgtaaagagatatatcctatacctgggccaaatgtaaacagcctggaaaagtgttaggttaaaaacaaaacaaaataaataaatgaataaatgccaggtggttatgagtgctattgagaaaaatgaagccaagagggatatcagtgatgcaggtgggggtaaagagcttacaacataaatgtggtgttccatatttaaacctcattcaacagggaagattggagctgaaatgtgaaggagttgtgggagtggaactacgtggaaatctgggggaaaggtgttttgggtaaaagaaatagcaagtgttgaggtccaggggcatgagtgtgcttgatattttagggaagagtaaggagaccagtataaccagagtgagatgagactacagaggtcaggagaaagggcatgcagaccatgtgggatgctctaggacctaggccatggtaaagatgtagggttttaccctgatggaggtcagaagccattggaggattctgagaagaggagtgacaggactcgctttatagttttaaattataactataaattatagtttttaaaacaatagttgcctaacctcatgttatatgtaaaactacagttttaaaaactataaattcctcatactggcagcagtgtgaggggcaagggcaaaagcagagagactaacaggttgctggttactcttgctagtgcaagtgaattctagaatcttcgacaacatccagaacttctcttgctgctgccactcaggaagagggttggagtaggctaggaataggagcacaaattaaagctcctgttcactttgacttctccatccctctcctcctttccttaaaggttctgattaaagcagacttatgcccctactgctctcagaagtgaatgggttaagtttagcagcctcccttttgctacttcagttcttcctgtggctgcttcccactgataaaaaggaagcaatcctatcggttactgcttagtgctgagcacatccagtgggtaaagttccttaaaatgctctgcaaagaaattgggacttttcattaaatcagaaattttacttttttcccctcctgggagctaaagatattttagagaagaattaaccttttgcttctccagttgaacatttgtagcaataagtc

SEQ ID NO: 341 - DNA, 734 bp, Synthetic construct
  source 1..734: mol_type = other DNA; note = Endogenous F8 promoter 2
tgggatgctctaggacctaggccatggtaaagatgtagggttttaccctgatggaggtcagaagccattggaggattctgagaagaggagtgacaggactcgctttatagttttaaattataactataaattatagtttttaaaacaatagttgcctaacctcatgttatatgtaaaactacagttttaaaaactataaattcctcatactggcagcagtgtgaggggcaagggcaaaagcagagagactaacaggttgctggttactcttgctagtgcaagtgaattctagaatcttcgacaacatccagaacttctcttgctgctgccactcaggaagagggttggagtaggctaggaataggagcacaaattaaagctcctgttcactttgacttctccatccctctcctcctttccttaaaggttctgattaaagcagacttatgcccctactgctctcagaagtgaatgggttaagtttagcagcctcccttttgctacttcagttcttcctgtggctgcttcccactgataaaaaggaagcaatcctatcggttactgcttagtgctgagcacatccagtgggtaaagttccttaaaatgctctgcaaagaaattgggacttttcattaaatcagaaattttacttttttcccctcctgggagctaaagatattttagagaagaattaaccttttgcttctccagttgaacatttgtagcaataagtc

SEQ ID NO: 342 - DNA, 424 bp, Synthetic construct
  source 1..424: mol_type = other DNA; note = Endogenous F8 promoter 3
gaacttctcttgctgctgccactcaggaagagggttggagtaggctaggaataggagcacaaattaaagctcctgttcactttgacttctccatccctctcctcctttccttaaaggttctgattaaagcagacttatgcccctactgctctcagaagtgaatgggttaagtttagcagcctcccttttgctacttcagttcttcctgtggctgcttcccactgataaaaaggaagcaatcctatcggttactgcttagtgctgagcacatccagtgggtaaagttccttaaaatgctctgcaaagaaattgggacttttcattaaatcagaaattttacttttttcccctcctgggagctaaagatattttagagaagaattaaccttttgcttctccagttgaacatttgtagcaataagtc

SEQ ID NO: 343 - DNA, 7035 bp, Synthetic construct
  source 1..7035: mol_type = other DNA
gaattcgagctcggtagccgaagagcgcgctcgctatctcactgaggccgggcgaccaaaggtcgcccgacgcccgggctttgcccgggcggcctcagtgagatagcgagcgcgctcttcgggagtggccaaaaccaactagacaactttgtatatctagagctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatcgatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagacacccacacaaaggaaaagggcctttccgtcctcagccgtcgcttcatgtgacaccacggagtaccgggcgccgtccaggcacctcgattagttctcgatcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagcggccgcggcaggctgccaccatgcagattgaactgtctacctgcttctttctgtgcctcctgaggttctgttttagtgccacaaggagatattacctaggagcagttgagctgtcctgggattacatgcagagtgatctgggggagctgccagttgatgctaggttccctccaagggtgcctaagtccttcccctttaatacctcagtggtgtataagaagaccctttttgtagagttcactgatcacctgttcaatatagctaaacccaggccaccttggatggggttgttgggcccaacaattcaggcagaggtttatgacacagtagtgataaccctcaagaatatggctagtcatcctgtgagcctgcatgcagtaggtgtgtcttactggaaggcttcagagggtgctgagtatgatgatcagacttctcagagagagaaggaagatgacaaagtctttccagggggatcacatacatatgtttggcaggttcttaaagagaatggtcctatggccagtgaccccctgtgtctgacttactcctacctgagccatgtggacctggtgaaggatctgaactctgggctgataggtgcactgctggtgtgtagggaaggtagccttgccaaagagaagacccaaacactccacaagttcatcttgctctttgcagtttttgatgaaggaaaaagctggcatagtgaaaccaaaaactctttgatgcaggatagggatgcagctagtgctagggcctggccaaagatgcacacagtcaatggttatgtgaacagaagtcttccaggtctgataggatgccacaggaaaagtgtgtactggcatgtgataggcatgggaaccaccccagaagtgcactcaatattccttgagggtcataccttccttgtgagaaatcataggcaggcatccttggagatcagtcccatcaccttcttaactgcccaaacccttctgatggaccttggtcaatttctgctgtcctgccatatttcttcccatcagcatgatgggatggaggcctatgtaaaggtggattcttgcccagaggaaccccagctgaggatgaagaacaatgaggaagcagaggattatgatgatgatcttactgatagtgagatggatgtggttaggtttgatgatgacaacagccccagctttatccagattaggtctgttgctaagaagcatccaaagacatgggttcattacatagctgcagaagaagaggactgggattatgcccccctagtccttgccccagatgatagatcctataaatctcagtatctgaacaatggacctcagaggataggcaggaagtataagaaagtgagattcatggcatatacagatgagaccttcaagaccagagaagcaatccaacatgaaagtgggattctgggcccactgctgtatggtgaggtgggtgacaccctgctgataatcttcaagaatcaggccagcaggccttataacatctatccacatgggataacagatgtaaggcctctgtatagtagaaggctgcccaaaggggtgaaacacctgaaagactttcctattcttccaggtgaaatcttcaaatacaagtggacagtcacagtggaggatgggcctactaaaagtgaccccaggtgcctgactaggtactatagttcctttgttaacatggaaagggacctagctagtgggcttataggcccccttctgatctgttataaagaatctgttgatcagaggggcaaccagatcatgtcagacaaaaggaatgtgatactttttagtgtgtttgatgagaataggagctggtacctgactgagaacatccagaggtttttgcccaacccagctggtgtgcagcttgaggacccagagtttcaagcctcaaacatcatgcacagtatcaatggctatgtttttgattctcttcagctgagtgtttgcctgcatgaagtagcttactggtacatcctctcaattggggctcagacagatttcctttcagtgttcttcagtggctacactttcaagcacaaaatggtctatgaggacactctgaccttattcccttttagtggagagacagtctttatgagtatggagaatcctgggctgtggattcttgggtgccacaactctgatttcagaaataggggaatgactgccctgctaaaggtgagtagttgtgacaaaaacacaggggattactatgaggacagctatgaggacatcagtgcctatctgctctcaaagaacaatgcaatagagcctaggtccttctcacagaatagcaggcatccctcaaccaggcaaaaacagtttaatgccaccaccataccagagaatgatattgaaaagactgacccttggtttgctcacaggaccccaatgcccaaaatccaaaatgtgtcttcaagtgacctgttaatgctgttgaggcagtcacccaccccacatgggctgtccctgtcagatcttcaagaggccaaatatgagaccttcagtgatgatccttcaccaggagccatagattctaacaactccctgtcagaaatgacacacttcagacctcagttacatcattctggggatatggtgttcacacctgaatctggtctccagctgaggttaaatgagaaacttgggactacagcagctactgagctgaaaaaacttgattttaaggtgagctcaacaagcaataacctgatttccaccattcctagtgacaacctagctgctggcactgataacacctcttctctgggccctccttcaatgccagtacattatgattcccaacttgataccaccctctttgggaagaagtctagcccacttacagaatcaggtggcccacttagtctctcagaggagaataatgattccaaacttttggagagtgggctgatgaactctcaagagagcagttgggggaagaatgtatcccagagagagataaccagaacaacccttcagtctgaccaggaggagatagattatgatgacactatctcagttgagatgaaaaaggaggactttgatatctatgatgaagatgaaaatcagtctcccaggtccttccagaaaaaaacaaggcattacttcatagcagcagtagagagactgtgggattatgggatgtcatccagtcctcatgtcctgaggaacagagctcagagtggctcagtgcctcaattcaagaaagtggtgttccaggagttcacagatggctccttcacacagcctctctacagaggagagctcaatgaacacttggggctgctggggccctatatcagggcagaggtggaggataatattatggtgacatttaggaaccaagcctcaaggccatactcattttattcttccctgatctcatatgaagaggatcagaggcagggggcagaacctaggaagaattttgtaaagcctaatgagactaagacttacttttggaaggtgcaacaccacatggctcccaccaaagatgaatttgattgcaaggcttgggcctacttctctgatgtggatctggagaaagatgtgcattctggcttgataggtcccctactggtttgtcatacaaataccctgaaccctgcacatggcaggcaggtgactgtgcaggagtttgctctcttcttcactatatttgatgagaccaagtcctggtacttcacagagaatatggaaagaaactgcagggccccatgtaatatccagatggaagaccccacctttaaggaaaattataggtttcatgctattaatggatatataatggacaccctcccaggtctggtcatggcacaggaccagaggataaggtggtatctgctttctatgggctctaatgaaaatattcatagcatccactttagtgggcatgtgttcactgtcaggaaaaaagaggagtataaaatggccctgtataacctgtacccaggagtgtttgagacagtagagatgcttcctagtaaggctggcatttggagagtagagtgcctgattggagaacatctccatgctggaatgagcaccctgttcctggtctatagtaacaagtgtcagaccccactgggcatggcctcaggtcatattagggacttccaaatcactgccagtggccaatatgggcagtgggcccccaaactagccaggttgcactattctgggagtattaatgcctggtctacaaaggaaccattttcctggatcaaagtggatctgctggccccaatgatcatacatggaattaagacccagggagccaggcagaagttttcctccctgtacattagtcagttcatcattatgtacagtttggatgggaaaaagtggcagacatataggggtaacagtacaggaacccttatggtgttctttggtaatgttgacagctcaggaatcaagcacaatattttcaaccccccaatcatagccaggtacatcaggctccaccctactcattatagcatcaggagcaccctgagaatggagcttatgggatgtgacctaaactcctgtagtatgcctctgggaatggaatcaaaggccatctctgatgcccaaattacagcctcatcctactttacaaacatgtttgctacttggtcaccatccaaagctagactgcacctgcaggggaggtcaaatgcctggaggccacaagtcaacaaccccaaagaatggctccaagttgactttcagaagactatgaaagtgactggggtgactactcaaggggtaaagtctcttcttaccagtatgtatgtcaaagagtttttgataagctcctcccaggatggccaccagtggacactgttttttcagaatggcaaggtgaaagtgttccaaggaaaccaggattcttttactccagtggtgaacagcctggaccccccccttctgaccagatacttgagaatccacccccagtcctgggttcatcagattgctctgaggatggaagtgctgggatgtgaggcccaggacctctactaaggatcctgacacaaagtgacgcgtcctagagctcgcactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggcgctagcgcaggaaccccttttaatggagggccactcccgaagagcgcgctcgctatctcactgaggccgggcgaccaaaggtcgcccgacgcccgggctttgcccgggcggcctcagtgagatagcgagcgcgctcttcgggcgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccggggaagagcc

SEQ ID NO: 344 - DNA, 117 bp, Synthetic construct
  source 1..117: mol_type = other DNA; note = L-ITR CpG Free
agaagagctctctgtacactcactcactcactgatccctggataccaaaggtatccagacacccagtctttgactgggtgggatcagtgagtgagtgagtgtacagagagctcttct

SEQ ID NO: 345 - DNA, 118 bp, Synthetic construct
  source 1..118: mol_type = other DNA; note = R-ITR CpG Free
gctcttcttctctgtacactcactcactcactgatcccacccagtcaaagactgggtgtctggatacctttggtatccagggatcagtgagtgagtgagtgtacagagaagaagagct

SEQ ID NO: 346 - DNA, 58 bp, Synthetic construct
  source 1..58: mol_type = other DNA; note = Aptamer
atccggctttaaacgggcaactgcgtctcattcacgttagagactacaaccgtcggat

SEQ ID NO: 347 - DNA, 544 bp, Synthetic construct
  source 1..544: mol_type = other DNA; note = ApoE enhancer-TTR promoter
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaactttgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctg

SEQ ID NO: 348-351 - skipped (4 entries)

SEQ ID NO: 352 - RNA, 23 nt, Synthetic construct
  source 1..23: mol_type = other RNA; note = AAV2 wt gRNA for Nicking Cas9
agcgagcgagcgcgcagagaggg

SEQ ID NO: 353 - DNA, 16 bp, Synthetic construct
  source 1..16: mol_type = other DNA; note = AAV2 wt gDNA for PfAgo
gctcgctcgctcggtg

SEQ ID NO: 354 - protein, 1670 aa, Synthetic construct
  source 1..1670: note = Truncated F8 v1
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLSCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 355 - protein, 2351 aa, Synthetic construct
  source 1..2351: note = Truncated F8 v2
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLSCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 356 - protein, 1457 aa, Synthetic construct
  source 1..1457: note = Truncated F8 v3
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLSCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKHHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 357 - protein, 1628 aa, Synthetic construct
  source 1..1628: note = Truncated F8 v4
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLSCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 358 - protein, 1658 aa, Synthetic construct
  source 1..1658: note = Truncated F8 (partial deletion of B domain and linker a3 )
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLSCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 359 - protein, 2351 aa, Homo sapiens
  source 1..2351: note = FVIII (accession number P00451)
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 360 - DNA, 4980 bp, Synthetic construct
  source 1..4980: mol_type = other DNA; note = Truncated F8 (partial deletion of B domain and linker a3 )
atgcaaatagagctctccacctgcttctttctgtgccttttgaggttctgctttagtgccaccagaagatactacctgggtgcagtggaactgtcatgggactatatgcaaagtgatctaggtgagctgcctgtggatgcaagatttcctcctagagtgccaaaatcttttccattcaacacctcagtagtgtacaaaaagactttatttgtagaatttactgatcaccttttcaacatagctaagccaaggccaccctggatgggtctgctaggtcctaccatccaggctgaggtttatgatacagtggtcattacacttaagaacatggcttcccatcctgtcagtcttcatgctgttggtgtatcctactggaaagctagtgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaatagtggcctcattggagccctactggtatgtagagaagggagtttagccaaggaaaagacacagaccttgcacaaatttatactactttttgctgtatttgatgaagggaaaagttggcactcagaaacaaagaactccttgatgcaggatagggatgctgcatctgctagggcctggcctaaaatgcacacagtcaatggttatgtaaacaggtctctgccaggtctgattggatgccacaggaaatcagtctattggcatgtgattggaatgggcaccactcctgaagtgcactcaatattcctagaaggtcacacatttcttgtgaggaaccataggcaggcctccttggaaatctccccaataactttccttactgctcaaacactcttgatggaccttggacagtttctactgtcttgtcatatctcttcccaccaacatgatggcatggaagcctatgtcaaagtagacagttgtccagaggaaccccaactaaggatgaaaaataatgaagaagctgaagactatgatgatgatcttactgattctgaaatggatgtggtcaggtttgatgatgacaactctccttcctttatccaaattaggtcagttgccaagaagcatcctaaaacttgggtacattacatagctgctgaagaggaggactgggactatgctcccttagtcctagccccagatgacagaagttataaaagtcaatatttgaacaatggccctcagaggattggtaggaagtacaaaaaagtcaggtttatggcatacacagatgaaacctttaagactagggaagctattcagcatgaatcaggaatcttgggacctttactttatggggaagttggagacacactgttgattatatttaagaatcaagcaagcagaccatataacatctaccctcatggaatcactgatgtcaggcctttgtattcaaggagattaccaaaaggtgtaaaacatttgaaggattttccaattctgccaggagaaatattcaaatataaatggacagtgactgtagaagatgggccaactaaatcagatcctaggtgcctgaccaggtattactctagttttgttaatatggagagagatctagcttcaggactaattggccctctcctcatctgctacaaagaatctgtagatcaaagaggaaaccagataatgtcagacaagaggaatgtcatcctgttttctgtatttgatgagaacaggagctggtacctcacagagaatatacaaaggtttctccccaatccagcaggagtgcagcttgaggacccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtctatggaaaacccaggtctatggattctggggtgccacaactcagactttaggaacagaggcatgacagccttactgaaggtttctagttgtgacaagaacactggtgattattatgaggacagttatgaagatatttcagcatacttgctgagtaaaaacaatgccattgaaccaagaagtttctcccagaatagtagacaccctagcactaggcaaaagcaatttaatgccaccacaattccagaaaatgacatagagaagactgacccttggtttgcacacagaacacctatgcctaaaatacaaaatgtctcctctagtgatttgttgatgctcttgaggcagagccctactccacatgggctatccttatctgatctccaagaagccaaatatgagactttttctgatgatccatcacctggagcaatagacagtaataacagcctgtctgaaatgacacacttcaggccacagctccatcacagtggggacatggtatttacccctgagagtggcctccaattaagattaaatgagaaactggggacaactgcagcaacagagttgaagaaacttgatttcaaagtttctagtacatcaaataatctgatttcaacaattccatcagacaatttggcagcaggtactgataatacaagttccttaggacccccaagtatgccagttcattatgatagtcaattagataccactctatttggcaaaaagtcatctccccttactgagagtggtggacctctgagcttgagtgaagaaaataatgattcaaagttgttagaatcaggtttaatgaatagccaagaaagttcatggggaaaaaatgtatctcaagaggaaattgactatgatgataccatatcagttgaaatgaagaaggaagattttgacatttatgatgaggatgaaaatcagagccccaggagctttcaaaagaaaacaaggcactattttattgctgcagtggagaggctctgggattatgggatgagtagctccccacatgttctaagaaacagggctcagagtggcagtgtccctcagttcaagaaagttgttttccaggaatttactgatggctcctttactcagcccttatacaggggagaactaaatgaacatttgggactactggggccatatataagagcagaagttgaagataatatcatggtaactttcagaaatcaggcctctaggccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattataggttccatgcaatcaatggctacataatggatacactacctggcttagtaatggctcaggatcaaaggattaggtggtatctgctcagcatgggcagtaatgaaaacatccattctattcatttcagtggacatgtgttcactgtaaggaaaaaagaggagtataaaatggcactgtacaatctctatccaggtgtttttgagacagtggaaatgttaccatccaaagctggaatttggagggtggaatgccttattggagagcatctacatgctgggatgagcacactttttctggtgtacagcaataagtgtcagacccccctgggaatggcttctggacacattagagattttcagattacagcttcaggacaatatggacagtgggccccaaagctggccagacttcattattcaggatcaatcaatgcctggagcaccaaggagcccttttcttggatcaaggtggatctgttggcaccaatgattattcatggcatcaagacccagggtgccaggcagaagttctccagcctctacatctctcagtttatcatcatgtatagtcttgatgggaagaagtggcagacttataggggaaattccactggaaccttaatggtcttctttggaaatgtggattcatctgggataaaacacaatatttttaaccctccaattattgctaggtacatcaggttgcacccaactcattatagcattaggagcactcttaggatggagttgatgggctgtgatttaaatagttgcagcatgccattgggaatggagagtaaagcaatatcagatgcacagattactgcttcatcctactttaccaatatgtttgccacctggtctccttcaaaagctaggcttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactttattttttcagaatggcaaagtaaaggtttttcagggaaatcaagacagtttcacacctgtggtgaactctctggacccacccttactgactaggtaccttaggattcacccccagagttgggtgcaccagattgccctgaggatggaggttctgggctgtgaggcacaggacctctactgatga

SEQ ID NO: 361 - DNA, 1636 bp, Synthetic construct
  source 1..1636: mol_type = other DNA; note = Construct 1
tgcgcgactcgctcgctcactgaggccgggcgaccaaaggtcgcccgacgcccgggctttgcccgggcggcctcagtgagcgagcgagtcgcgcagagaggttaaaaccaactagacaactttgtatatctagagttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggcaagtttgtacaaaaaagcgcggccgcggcaggctgccaccatggaaaccgatacactgctgctttgggtacttttgctgtgggtgcccggcagtacgggcgacgccgcacaaccagctaggagagcagtccggagcctggtcccctcttccgaggccgaggctgaacggggaaagctgccaggcaagaaactccccttagaggttctcatcgagctggaagcaaacgcacgcaaggccggttgcactaggggctgcctcatctgcctcagcaagatcaaatgtacagcaaagatgaagaagtatattcctggccgctgtgcagactacggaggcgacaaaaaaacaggacaagctggtatagttggcgccatcgtagatatccccgagattagtgggttcaaggaaatggagccaatggagcaattcattgcacaggttgatagatgtgcggattgcactactggttgccttaagggcttggcgaatgtcaagtgtagtgatcttctgaaaaagtggctacccggccggtgtgccaccttcgctgacaaaatacagagcgaagtcgacaatattaaaggacttgccggggactaatgatagtgacacaaagtgacgcgtcctagagctcgcactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggcgctagcgcaggaaccccttttaatggagttggcgagtccctctctgcgcgctcgctcgctcactgaggccgggcgaccaaaggtcgcccgacgcccgggctttgcccgggcggcctcagtgagcgagcgagcgcgcagagatcgactc

SEQ ID NO: 362 - DNA, 1796 bp, Synthetic construct
  source 1..1796: mol_type = other DNA; note = Construct 2
tgcgcgactcgctcgctcactgaggccgggcgaccaaaggtcgcccgacgcccgggctttgcccgggcggcctcagtgagcgagcgagtcgcgcagagaggttaaaaccaactagacaactttgtatatctagagttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggcaagtttgtacaaaaaagcgcggccgcggcaggctgccaccatggaaaccgatacactgctgctttgggtacttttgctgtgggtgcccggcagtacgggcgacgccgcacaaccagctaggagagcagtccggagcctggtcccctcttccgaggccgaggctgaacggggaaagctgccaggcaagaaactccccttagaggttctcatcgagctggaagcaaacgcacgcaaggccggttgcactaggggctgcctcatctgcctcagcaagatcaaatgtacagcaaagatgaagaagtatattcctggccgctgtgcagactacggaggcgacaaaaaaacaggacaagctggtatagttggcgccatcgtagatatccccgagattagtgggttcaaggaaatggagccaatggagcaattcattgcacaggttgatagatgtgcggattgcactactggttgccttaagggcttggcgaatgtcaagtgtagtgatcttctgaaaaagtggctacccggccggtgtgccaccttcgctgacaaaatacagagcgaagtcgacaatattaaaggacttgccggggactaatgatagtgacacaaagtgacgcgtcctagagctcgcactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggcgctagcgcaggaaccccttttaatggagttggcgagtccctctctgcgcgctcgctcgctcactgaggccgggcgaccaaaggtcgcccgacgcccgggctttgcccgggcggcctcagtgagcgagcgagcgcgcagagatcgactcctcggccacttggaggggccggggggacgacgcaatctggagtggaaagaacccccgtctatgcggcttaaagcacggccagggaatagtggatcaagtgtactgacatgtgccggagtccctccatgcccagatcgactccctcgagatatatggatcc

SEQ ID NO: 363 - DNA, 16 bp, Synthetic construct
  source 1..16: mol_type = other DNA; note = Rep (AAV1,2,7)
gcgcgctcgctcgctc

SEQ ID NO: 364 - DNA, 17 bp, Synthetic construct
  source 1..17: mol_type = other DNA; note = Rep (AAV3)
tgcgcactcgctcgctc

SEQ ID NO: 365 - DNA, 16 bp, Synthetic construct
  source 1..16: mol_type = other DNA; note = Rep (AAV4)
gcgcgctcgctcactc

SEQ ID NO: 366 - DNA, 20 bp, Synthetic construct
  source 1..20: mol_type = other DNA; note = Rep (AAV5)
gttcgctcgctcgctggctc

SEQ ID NO: 367 - DNA, 10 bp, Synthetic construct
  source 1..10: mol_type = other DNA; note = NS1-NSBE3 (B19V)
ttccggtaca

SEQ ID NO: 368 - DNA, 120 bp, Synthetic construct
  source 1..120: mol_type = other DNA; note = Plasmid 1 AAV2 Left ITR RBE
gagtccctctctgcgcgctcgctcgctcactgaggccgggcgaccaaaggtcgcccgacgcccgggctttgcccgggcggcctcagtgagcgagcgagcgcgcagagagggactcgccaa

SEQ ID NO: 369 - DNA, 120 bp, Synthetic construct
  source 1..120: mol_type = other DNA; note = Plasmid 1 AAV2 Right ITR RBE
ttggcgagtccctctctgcgcgctcgctcgctcactgaggccgggcgaccaaaggtcgcccgacgcccgggctttgcccgggcggcctcagtgagcgagcgagcgcgcagagagggactc

SEQ ID NO: 370 - DNA, 120 bp, Synthetic construct
  source 1..120: mol_type = other DNA; note = Plasmid 2 ITR1 (Left ITR)
gagtccctctctgcgcggtccttcgctcactgaggccgggcgaccaaaggtcgcccgacgcccgggctttgcccgggcggcctcagtgagcgaaggaccgcgcagagagggactcgccaa

SEQ ID NO: 371 - DNA, 120 bp, Synthetic construct
  source 1..120: mol_type = other DNA; note = Plasmid 2 ITR1 (Right ITR)
ttggcgagtccctctctgcgcggtccttcgctcactgaggccgggcgaccaaaggtcgcccgacgcccgggctttgcccgggcggcctcagtgagcgaaggaccgcgcagagagggactc

SEQ ID NO: 372 - DNA, 120 bp, Synthetic construct
  source 1..120: mol_type = other DNA; note = Plasmid 3 ITR2 (Left ITR)
gagtccctctctgcgcgctccttcggtcactgaggccgggcgaccaaaggtcgcccgacgcccgggctttgcccgggcggcctcagtgaccgaaggagcgcgcagagagggactcgccaa

SEQ ID NO: 373 - DNA, 120 bp, Synthetic construct
  source 1..120: mol_type = other DNA; note = Plasmid 3 ITR2 (Right ITR)
ttggcgagtccctctctgcgcgctccttcggtcactgaggccgggcgaccaaaggtcgcccgacgcccgggctttgcccgggcggcctcagtgaccgaaggagcgcgcagagagggactc

SEQ ID NO: 374 - DNA, 120 bp, Synthetic construct
  source 1..120: mol_type = other DNA; note = Plasmid 4 ITR3 (Left ITR)
gagtccctctctgcgcgatcactcactcactgaggccgggcgaccaaaggtcgcccgacgcccgggctttgcccgggcggcctcagtgagtgagtgatcgcgcagagagggactcgccaa

SEQ ID NO: 375 - DNA, 120 bp, Synthetic construct
  source 1..120: mol_type = other DNA; note = Plasmid 4 ITR3 (Right ITR)
ttggcgagtccctctctgcgcgatcactcactcactgaggccgggcgaccaaaggtcgcccgacgcccgggctttgcccgggcggcctcagtgagtgagtgatcgcgcagagagggactc

SEQ ID NO: 376 - DNA, 120 bp, Synthetic construct
  source 1..120: mol_type = other DNA; note = Plasmid 5 ITR4 (Left ITR)
gagtccctctctgcgcgcccgaacgcacactgaggccgggcgaccaaaggtcgcccgacgcccgggctttgcccgggcggcctcagtgtgcgttcgggcgcgcagagagggactcgccaa

SEQ ID NO: 377 - DNA, 120 bp, Synthetic construct
  source 1..120: mol_type = other DNA; note = Plasmid 5 ITR4 (Right ITR)
ttggcgagtccctctctgcgcgcccgaacgcacactgaggccgggcgaccaaaggtcgcccgacgcccgggctttgcccgggcggcctcagtgtgcgttcgggcgcgcagagagggactc

SEQ ID NO: 378 - protein, 1457 aa, Synthetic construct
  source 1..1457: note = Truncated FVIIIv11
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 379 - DNA, 4374 bp, Synthetic construct
  source 1..4374: mol_type = other DNA; note = Gene codifying FVIII v12
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcttctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggatctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcttatgtgaaggtggatagctgccctgaggagccccaactgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcaactggaggaccctgagttccaggccagcaacatcatgcacagcatcaatggctatgtgtttgacagcctgcaactgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctctagctgtgacaagaacactggggactactatgaggacagctatgaggacatctctgcctacctgctgagcaagaacaatgccattgagcccaggagcttcagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcttgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagccctttagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaatagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagtctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactag

SEQ ID NO: 380 - DNA, 4374 bp, Synthetic construct
  source 1..4374: mol_type = other DNA; note = Gene codifying FVIII v13
atgcagatcgagctgtccacctgcttcttcctgtgcctgctgcggttctgcttttctgccacacggagatactatctgggagccgtggagctgtcttgggattacatgcagagcgacctgggagagctgccagtggacgcaaggtttccacctagagtgcctaagtccttccccttcaacacctctgtggtgtataagaaaaccctgttcgtggagtttacagatcacctgttcaacatcgccaagccaaggccaccatggatgggcctgctgggaccaacaatccaggcagaggtgtacgacaccgtggtcatcaccctgaagaacatggccagccaccccgtgagcctgcacgcagtgggcgtgtcctactggaaggccagcgagggagccgagtatgacgatcagacctctcagcgcgagaaggaggacgataaggtgtttccaggcggcagccacacatacgtgtggcaggtgctgaaggagaatggccccatggccagcgatcctctgtgcctgacctacagctatctgtcccacgtggatctggtgaaggatctgaacagcggcctgatcggggccctgctggtgtgcagagagggctccctggccaaggagaaaacccagacactgcacaagttcatcctgctgttcgccgtgtttgacgagggcaagtcttggcacagcgagacaaagaattctctgatgcaggacagggatgccgcaagcgccagggcatggccaaagatgcacacagtgaacggctacgtgaataggtccctgcccggcctgatcggctgtcacagaaagagcgtgtactggcacgtgatcggcatgggcaccacaccagaggtgcactccatcttcctggagggccacacctttctggtgaggaaccacaggcaggccagcctggagatcagccccatcaccttcctgacagcccagaccctgctgatggatctgggccagttcctgctgttttgccacatcagctcccaccagcacgatggcatggaagcctacgtgaaggtggactcctgccccgaggagccccagctcagaatgaagaacaatgaggaggccgaggactatgacgatgacctgaccgactccgagatggatgtggtgcggttcgatgacgataacagcccatcctttatccagatcagaagcgtggccaagaagcaccccaagacatgggtgcactacatcgccgccgaggaggaggactgggattatgccccactggtgctggcccccgacgatagatcctacaagtctcagtatctgaacaatggcccccagcggatcggcagaaagtacaagaaggtgaggttcatggcctatacagatgagacattcaagacacgcgaggccatccagcacgagagcggcatcctgggacctctgctgtacggagaagtgggcgacaccctgctgatcatctttaagaaccaggccagccggccctacaatatctatccacacggcatcacagatgtgcgccctctgtacagccggcggctgcccaagggagtgaagcacctgaaggacttcccaatcctgcccggcgagatctttaagtataagtggaccgtgacagtggaggatggccccaccaagtccgaccctaggtgcctgacacgctactattctagcttcgtgaatatggagagggatctggcaagcggcctgatcggacctctgctgatctgttacaaggagagcgtggatcagcggggcaaccagatcatgtccgacaagagaaatgtgatcctgttcagcgtgttcgacgagaaccggagctggtatctgaccgagaacatccagagattcctgcctaatccagccggcgtgcagctcgaggatccagagtttcaggccagcaacatcatgcactccatcaatggctacgtgttcgacagcctgcagctcagcgtgtgcctgcacgaggtggcctactggtatatcctgtccatcggagcccagaccgatttcctgagcgtgttcttttccggctacaccttcaagcacaagatggtgtatgaggacaccctgacactgttccctttttccggcgagacagtgttcatgtctatggagaatcccggcctgtggatcctgggctgccacaacagcgacttcagaaacagaggcatgaccgccctgctgaaggtgtcctcttgtgacaagaacacaggcgactactatgaggattcttacgaggacatcagcgcctatctgctgtccaagaacaatgccatcgagcctcgctcttttagccagaatcctccagtgctgaagcggcaccagcgggagatcaccagaaccacactgcagtctgatcaggaggagatcgactacgacgatacaatcagcgtggagatgaagaaggaggacttcgacatctacgacgaggatgagaaccagagccccaggtccttccagaagaaaacccgccactactttatcgccgccgtggagagactgtgggattatggcatgtcctctagcccccacgtgctgaggaatcgcgcccagtccggctctgtgcctcagttcaagaaggtggtgttccaggagtttaccgacggctcctttacacagcctctgtacaggggagagctgaacgagcacctgggcctgctgggaccatatatcagagccgaggtggaggataacatcatggtgaccttcaggaatcaggccagccggccctatagcttttattcctctctgatcagctacgaggaggaccagaggcagggagccgagccaagaaagaacttcgtgaagcccaatgagacaaagacctacttttggaaggtgcagcaccacatggcccccaccaaggacgagttcgattgcaaagcctgggcctatttttctgacgtggatctggagaaggacgtgcacagcggcctgatcggaccactgctggtgtgccacaccaacacactgaatccagcacacggccggcaggtcaccgtgcaggagttcgccctgttctttaccatctttgatgagacaaagagctggtacttcacagagaacatggagaggaattgccgcgccccttgtaatatccagatggaggacccaaccttcaaggagaactacagatttcacgccatcaatggctatatcatggatacactgcccggcctggtcatggcacaggaccagcggatcagatggtatctgctgtctatgggcagcaacgagaatatccactccatccacttctctggccacgtgtttaccgtgaggaagaaggaggagtataagatggccctgtacaacctgtatcccggcgtgttcgagacagtggagatgctgcctagcaaggcaggaatctggagggtggagtgcctgatcggagagcacctgcacgcaggcatgtctaccctgtttctggtgtacagcaataagtgtcagacacctctgggaatggcaagcggccacatcagggatttccagatcaccgcatccggccagtacggacagtgggccccaaagctggccagactgcactatagcggctccatcaacgcctggagcaccaaggagcctttctcctggatcaaggtggacctgctggccccaatgatcatccacggcatcaagacacagggagcccggcagaagttcagctccctgtacatctcccagtttatcatcatgtattctctggatggcaagaagtggcagacctacagaggcaatagcaccggcacactgatggtgttctttggcaacgtggactctagcggcatcaagcacaacatcttcaatccccctatcatcgcccggtacatcagactgcaccccacccactattccatcaggtctacactgcgcatggagctgatgggctgcgatctgaactcttgtagcatgcctctgggcatggagagcaaggccatctccgacgcccagatcaccgcctcctcttacttcaccaatatgtttgccacatggtccccctctaaggccaggctgcacctgcagggccggtccaacgcatggcggccccaggtgaacaatcccaaggagtggctgcaggtggattttcagaaaaccatgaaggtgacaggcgtgaccacacagggcgtgaagtctctgctgaccagcatgtatgtgaaggagttcctgatcagctcctctcaggacggccaccagtggacactgttctttcagaacggcaaggtgaaggtgttccagggcaatcaggatagctttaccccagtggtgaactccctggacccaccactgctgaccagatacctgcggatccaccctcagagctgggtgcaccagatcgccctgcgcatggaggtgctgggatgcgaggcacaggacctgtattag

SEQ ID NO: 381 - DNA, 4374 bp, Synthetic construct
  source 1..4374: mol_type = other DNA; note = Gene codifying FVIII v14
atgcagatagaactgagcacgtgcttttttttatgtctgctgcgattttgcttcagcgctacccgtcgatattacctgggggctgttgaattgtcttgggattacatgcagtcagatttaggcgaactccctgtggacgcccggttccccccccgcgtcccaaaaagcttcccttttaacacttccgttgtgtataagaagactttatttgtcgagtttacagaccacctctttaatattgccaagcccaggccaccctggatgggactgctcgggcctaccatccaggctgaggtgtatgacacggtggtgatcaccctcaaaaacatggccagccacccggtgtcactccacgctgtcggagtgtcttactggaaggcctccgaaggggccgagtacgatgaccagacctctcagagggagaaagaggacgataaggttttccccggcgggtcccacacttatgtgtggcaggttctgaaggagaacggcccaatggcgtccgatccattgtgtttgacttactcctacttgtctcacgtcgatctggttaaagacttaaactcaggcctgataggggcccttctcgtgtgtagagaaggtagcctcgctaaagagaagactcagaccctgcacaagtttattctgctctttgccgtgtttgatgagggtaagagctggcactccgaaacgaaaaattccctcatgcaggaccgggacgccgcttcagctcgggcctggcctaagatgcacacagtgaacgggtacgtgaatcgcagcctgcctggtttgatcggctgccatcgaaagagcgtgtattggcacgtgatcggaatgggcacaacccctgaggttcatagcatcttcttggagggccatacattcctggttaggaatcataggcaggcttctttggagatcagccccatcacctttctgacagcacaaacactgctgatggacctcggacaatttttgctgttttgtcacatctctagtcaccaacacgacgggatggaagcctatgtcaaagtggactcctgccccgaagaaccccagctccggatgaagaataatgaagaagccgaagactatgatgacgacttgaccgactccgagatggacgtggtgaggtttgacgatgataactctccttcctttatacagatcaggagtgtggctaaaaagcatccgaagacctgggtgcactatatcgccgcagaggaggaagattgggactatgccccactcgtgctggctcctgacgaccgcagttacaaatctcagtacctcaataacggcccccagaggattggtcgaaaatataagaaggtcagatttatggcctatacggacgagacattcaaaacaagggaagccatccagcacgaatccggcatcttgggcccactcctgtacggagaagtcggtgacaccctcctgattatttttaagaaccaggccagccgcccttacaatatatacccccatggcatcaccgatgttaggcctctgtattccaggcgcctgcccaaaggggtaaagcaccttaaagattttcccatcctgcccggcgaaatctttaagtataagtggaccgtgactgttgaagatgggcccactaaatcagatccaaggtgcctgactcggtattacagcagtttcgtcaatatggagagggatctcgcatcaggcctgatcggacctctgctgatctgctataaggagtccgtagatcagaggggaaatcagattatgagcgataagagaaatgtgattctgtttagtgtgtttgatgaaaaccgctcatggtacctgacggagaatatccagcgcttcctcccaaatcccgcaggcgtccagctcgaagaccccgagttccaggccagtaatatcatgcacagcatcaacgggtatgtttttgactccttgcagttgtccgtctgtctgcacgaggtcgcctactggtatattcttagcatcggagctcagacagatttcctcagcgttttctttagtgggtatacatttaagcacaaaatggtatacgaggatactctcaccttgttccctttctcaggcgagactgtctttatgtctatggaaaatcccggcctctggattttagggtgtcataattctgatttccgcaacagaggcatgacagccctgttaaaggtttcctcctgcgataagaatacaggcgattattatgaggattcttacgaggatattagtgcgtatttgctgagtaagaacaacgccatcgagccaaggagcttctcccagaacccgccggttttgaaaagacatcaaagagagatcacacggaccacgctgcaatcagatcaggaggagattgattacgacgatactattagcgtggaaatgaagaaagaagatttcgatatatacgacgaggatgaaaaccagtcccctcgcagcttccagaaaaagaccaggcactattttatcgctgccgtggagagactgtgggattacggtatgagtagtagcccacatgtgttgcgaaaccgcgcacagtctggatctgttccccagtttaaaaaggtggtgtttcaagagttcaccgacgggtcttttacccagcctctgtaccgaggtgaactcaacgagcacctcgggctcctgggcccatatattcgagctgaggtagaggataacatcatggtgacatttaggaaccaagcctctcgaccatactccttctacagctcactcatcagctatgaggaggaccaacggcaaggtgccgaaccccggaagaatttcgtgaagcctaatgaaaccaagacatacttctggaaagttcaacatcatatggctcctactaaagacgaatttgattgtaaggcctgggcgtactttagcgacgtcgatctggaaaaggacgtgcatagcgggctcattggtccgcttttggtttgccacaccaacacactgaaccccgctcacgggcgccaggtcaccgtacaggagtttgcactcttcttcaccatattcgatgagactaagtcttggtatttcacagaaaatatggagcgaaactgccgggccccgtgcaacattcagatggaagacccaacgtttaaggagaactaccgctttcacgctattaatgggtacatcatggatacattgccaggcctggtgatggcccaggaccagaggattcgatggtacctgctgagcatgggtagcaatgaaaatattcacagcatccacttctccggtcacgtattcactgtgcgaaagaaagaagagtacaaaatggcgctttacaacctgtacccaggggtgtttgagacagtggagatgctgccaagtaaagccggcatctggagagtcgagtgcttgatcggcgagcatctccacgccggcatgtctactctgtttttagtatattcaaataaatgtcagacccccttagggatggcatctggccacattagggactttcaaattacagcctcaggtcagtacggtcagtgggcccctaaactggctcggcttcactattccggctcaattaatgcgtggtcaacaaaagagccgtttagttggataaaggttgacctgttagcccctatgatcatacatggcattaagacccagggggcacggcaaaagtttagctcactttacatcagtcaatttatcataatgtatagtttggacgggaaaaaatggcagacctaccggggaaattccaccgggacccttatggtattctttggtaatgttgattccagcggcatcaaacacaacatcttcaatcctccaatcatcgcccgctatattcggctgcatcctactcattacagcatccggtctacactgcggatggaactgatgggttgcgatctgaactcatgctccatgcctctcggcatggagtctaaagcaatttccgatgcgcagataactgccagctcatattttaccaacatgttcgctacttggagtccaagcaaggcacgcctgcatttacagggcaggtctaacgcctggcggcctcaggtgaataatccgaaagagtggcttcaggtggattttcagaaaacgatgaaggttactggtgtcacgacccaaggtgtgaagtcccttctgacgtccatgtatgtcaaggaattcttgatcagttcaagccaagacggacatcagtggaccctgttttttcaaaatggcaaggtgaaagtttttcaaggcaatcaggacagcttcacccctgttgttaacagccttgatccacccttactcacaagatacttgagaatccatccccagagttgggtgcaccaaatcgctctccggatggaggtgctgggttgtgaggcccaggacctgtactag

SEQ ID NO: 382 - protein, 1454 aa, Synthetic construct
  source 1..1454: note = Truncated FVIII v15
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 383 - DNA, 4365 bp, Synthetic construct
  source 1..4365: mol_type = other DNA; note = Gene codifying FVIII v16
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcttctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggatctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcttatgtgaaggtggatagctgccctgaggagccccaactgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcaactggaggaccctgagttccaggccagcaacatcatgcacagcatcaatggctatgtgtttgacagcctgcaactgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctctagctgtgacaagaacactggggactactatgaggacagctatgaggacatctctgcctacctgctgagcaagaacaatgccattgagcccaggagcttcagccagaaccccccagtgctgaagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcttgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagccctttagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaatagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagtctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactag

SEQ ID NO: 384 - DNA, 4365 bp, Synthetic construct
  source 1..4365: mol_type = other DNA; note = Gene codifying FVIII v17
atgcagatcgagctgtccacctgcttcttcctgtgcctgctgcggttctgcttttctgccacacggagatactatctgggagccgtggagctgtcttgggattacatgcagagcgacctgggagagctgccagtggacgcaaggtttccacctagagtgcctaagtccttccccttcaacacctctgtggtgtataagaaaaccctgttcgtggagtttacagatcacctgttcaacatcgccaagccaaggccaccatggatgggcctgctgggaccaacaatccaggcagaggtgtacgacaccgtggtcatcaccctgaagaacatggccagccaccccgtgagcctgcacgcagtgggcgtgtcctactggaaggccagcgagggagccgagtatgacgatcagacctctcagcgcgagaaggaggacgataaggtgtttccaggcggcagccacacatacgtgtggcaggtgctgaaggagaatggccccatggccagcgatcctctgtgcctgacctacagctatctgtcccacgtggatctggtgaaggatctgaacagcggcctgatcggggccctgctggtgtgcagagagggctccctggccaaggagaaaacccagacactgcacaagttcatcctgctgttcgccgtgtttgacgagggcaagtcttggcacagcgagacaaagaattctctgatgcaggacagggatgccgcaagcgccagggcatggccaaagatgcacacagtgaacggctacgtgaataggtccctgcccggcctgatcggctgtcacagaaagagcgtgtactggcacgtgatcggcatgggcaccacaccagaggtgcactccatcttcctggagggccacacctttctggtgaggaaccacaggcaggccagcctggagatcagccccatcaccttcctgacagcccagaccctgctgatggatctgggccagttcctgctgttttgccacatcagctcccaccagcacgatggcatggaagcctacgtgaaggtggactcctgccccgaggagccccagctcagaatgaagaacaatgaggaggccgaggactatgacgatgacctgaccgactccgagatggatgtggtgcggttcgatgacgataacagcccatcctttatccagatcagaagcgtggccaagaagcaccccaagacatgggtgcactacatcgccgccgaggaggaggactgggattatgccccactggtgctggcccccgacgatagatcctacaagtctcagtatctgaacaatggcccccagcggatcggcagaaagtacaagaaggtgaggttcatggcctatacagatgagacattcaagacacgcgaggccatccagcacgagagcggcatcctgggacctctgctgtacggagaagtgggcgacaccctgctgatcatctttaagaaccaggccagccggccctacaatatctatccacacggcatcacagatgtgcgccctctgtacagccggcggctgcccaagggagtgaagcacctgaaggacttcccaatcctgcccggcgagatctttaagtataagtggaccgtgacagtggaggatggccccaccaagtccgaccctaggtgcctgacacgctactattctagcttcgtgaatatggagagggatctggcaagcggcctgatcggacctctgctgatctgttacaaggagagcgtggatcagcggggcaaccagatcatgtccgacaagagaaatgtgatcctgttcagcgtgttcgacgagaaccggagctggtatctgaccgagaacatccagagattcctgcctaatccagccggcgtgcagctcgaggatccagagtttcaggccagcaacatcatgcactccatcaatggctacgtgttcgacagcctgcagctcagcgtgtgcctgcacgaggtggcctactggtatatcctgtccatcggagcccagaccgatttcctgagcgtgttcttttccggctacaccttcaagcacaagatggtgtatgaggacaccctgacactgttccctttttccggcgagacagtgttcatgtctatggagaatcccggcctgtggatcctgggctgccacaacagcgacttcagaaacagaggcatgaccgccctgctgaaggtgtcctcttgtgacaagaacacaggcgactactatgaggattcttacgaggacatcagcgcctatctgctgtccaagaacaatgccatcgagcctcgctcttttagccagaatcctccagtgctgaagcgggagatcaccagaaccacactgcagtctgatcaggaggagatcgactacgacgatacaatcagcgtggagatgaagaaggaggacttcgacatctacgacgaggatgagaaccagagccccaggtccttccagaagaaaacccgccactactttatcgccgccgtggagagactgtgggattatggcatgtcctctagcccccacgtgctgaggaatcgcgcccagtccggctctgtgcctcagttcaagaaggtggtgttccaggagtttaccgacggctcctttacacagcctctgtacaggggagagctgaacgagcacctgggcctgctgggaccatatatcagagccgaggtggaggataacatcatggtgaccttcaggaatcaggccagccggccctatagcttttattcctctctgatcagctacgaggaggaccagaggcagggagccgagccaagaaagaacttcgtgaagcccaatgagacaaagacctacttttggaaggtgcagcaccacatggcccccaccaaggacgagttcgattgcaaagcctgggcctatttttctgacgtggatctggagaaggacgtgcacagcggcctgatcggaccactgctggtgtgccacaccaacacactgaatccagcacacggccggcaggtcaccgtgcaggagttcgccctgttctttaccatctttgatgagacaaagagctggtacttcacagagaacatggagaggaattgccgcgccccttgtaatatccagatggaggacccaaccttcaaggagaactacagatttcacgccatcaatggctatatcatggatacactgcccggcctggtcatggcacaggaccagcggatcagatggtatctgctgtctatgggcagcaacgagaatatccactccatccacttctctggccacgtgtttaccgtgaggaagaaggaggagtataagatggccctgtacaacctgtatcccggcgtgttcgagacagtggagatgctgcctagcaaggcaggaatctggagggtggagtgcctgatcggagagcacctgcacgcaggcatgtctaccctgtttctggtgtacagcaataagtgtcagacacctctgggaatggcaagcggccacatcagggatttccagatcaccgcatccggccagtacggacagtgggccccaaagctggccagactgcactatagcggctccatcaacgcctggagcaccaaggagcctttctcctggatcaaggtggacctgctggccccaatgatcatccacggcatcaagacacagggagcccggcagaagttcagctccctgtacatctcccagtttatcatcatgtattctctggatggcaagaagtggcagacctacagaggcaatagcaccggcacactgatggtgttctttggcaacgtggactctagcggcatcaagcacaacatcttcaatccccctatcatcgcccggtacatcagactgcaccccacccactattccatcaggtctacactgcgcatggagctgatgggctgcgatctgaactcttgtagcatgcctctgggcatggagagcaaggccatctccgacgcccagatcaccgcctcctcttacttcaccaatatgtttgccacatggtccccctctaaggccaggctgcacctgcagggccggtccaacgcatggcggccccaggtgaacaatcccaaggagtggctgcaggtggattttcagaaaaccatgaaggtgacaggcgtgaccacacagggcgtgaagtctctgctgaccagcatgtatgtgaaggagttcctgatcagctcctctcaggacggccaccagtggacactgttctttcagaacggcaaggtgaaggtgttccagggcaatcaggatagctttaccccagtggtgaactccctggacccaccactgctgaccagatacctgcggatccaccctcagagctgggtgcaccagatcgccctgcgcatggaggtgctgggatgcgaggcacaggacctgtattag

SEQ ID NO: 385 - DNA, 4365 bp, Synthetic construct
  source 1..4365: mol_type = other DNA; note = Gene codifying FVIII v18
atgcagatagaactgagcacgtgcttttttttatgtctgctgcgattttgcttcagcgctacccgtcgatattacctgggggctgttgaattgtcttgggattacatgcagtcagatttaggcgaactccctgtggacgcccggttccccccccgcgtcccaaaaagcttcccttttaacacttccgttgtgtataagaagactttatttgtcgagtttacagaccacctctttaatattgccaagcccaggccaccctggatgggactgctcgggcctaccatccaggctgaggtgtatgacacggtggtgatcaccctcaaaaacatggccagccacccggtgtcactccacgctgtcggagtgtcttactggaaggcctccgaaggggccgagtacgatgaccagacctctcagagggagaaagaggacgataaggttttccccggcgggtcccacacttatgtgtggcaggttctgaaggagaacggcccaatggcgtccgatccattgtgtttgacttactcctacttgtctcacgtcgatctggttaaagacttaaactcaggcctgataggggcccttctcgtgtgtagagaaggtagcctcgctaaagagaagactcagaccctgcacaagtttattctgctctttgccgtgtttgatgagggtaagagctggcactccgaaacgaaaaattccctcatgcaggaccgggacgccgcttcagctcgggcctggcctaagatgcacacagtgaacgggtacgtgaatcgcagcctgcctggtttgatcggctgccatcgaaagagcgtgtattggcacgtgatcggaatgggcacaacccctgaggttcatagcatcttcttggagggccatacattcctggttaggaatcataggcaggcttctttggagatcagccccatcacctttctgacagcacaaacactgctgatggacctcggacaatttttgctgttttgtcacatctctagtcaccaacacgacgggatggaagcctatgtcaaagtggactcctgccccgaagaaccccagctccggatgaagaataatgaagaagccgaagactatgatgacgacttgaccgactccgagatggacgtggtgaggtttgacgatgataactctccttcctttatacagatcaggagtgtggctaaaaagcatccgaagacctgggtgcactatatcgccgcagaggaggaagattgggactatgccccactcgtgctggctcctgacgaccgcagttacaaatctcagtacctcaataacggcccccagaggattggtcgaaaatataagaaggtcagatttatggcctatacggacgagacattcaaaacaagggaagccatccagcacgaatccggcatcttgggcccactcctgtacggagaagtcggtgacaccctcctgattatttttaagaaccaggccagccgcccttacaatatatacccccatggcatcaccgatgttaggcctctgtattccaggcgcctgcccaaaggggtaaagcaccttaaagattttcccatcctgcccggcgaaatctttaagtataagtggaccgtgactgttgaagatgggcccactaaatcagatccaaggtgcctgactcggtattacagcagtttcgtcaatatggagagggatctcgcatcaggcctgatcggacctctgctgatctgctataaggagtccgtagatcagaggggaaatcagattatgagcgataagagaaatgtgattctgtttagtgtgtttgatgaaaaccgctcatggtacctgacggagaatatccagcgcttcctcccaaatcccgcaggcgtccagctcgaagaccccgagttccaggccagtaatatcatgcacagcatcaacgggtatgtttttgactccttgcagttgtccgtctgtctgcacgaggtcgcctactggtatattcttagcatcggagctcagacagatttcctcagcgttttctttagtgggtatacatttaagcacaaaatggtatacgaggatactctcaccttgttccctttctcaggcgagactgtctttatgtctatggaaaatcccggcctctggattttagggtgtcataattctgatttccgcaacagaggcatgacagccctgttaaaggtttcctcctgcgataagaatacaggcgattattatgaggattcttacgaggatattagtgcgtatttgctgagtaagaacaacgccatcgagccaaggagcttctcccagaacccgccggttttgaaaagagagatcacacggaccacgctgcaatcagatcaggaggagattgattacgacgatactattagcgtggaaatgaagaaagaagatttcgatatatacgacgaggatgaaaaccagtcccctcgcagcttccagaaaaagaccaggcactattttatcgctgccgtggagagactgtgggattacggtatgagtagtagcccacatgtgttgcgaaaccgcgcacagtctggatctgttccccagtttaaaaaggtggtgtttcaagagttcaccgacgggtcttttacccagcctctgtaccgaggtgaactcaacgagcacctcgggctcctgggcccatatattcgagctgaggtagaggataacatcatggtgacatttaggaaccaagcctctcgaccatactccttctacagctcactcatcagctatgaggaggaccaacggcaaggtgccgaaccccggaagaatttcgtgaagcctaatgaaaccaagacatacttctggaaagttcaacatcatatggctcctactaaagacgaatttgattgtaaggcctgggcgtactttagcgacgtcgatctggaaaaggacgtgcatagcgggctcattggtccgcttttggtttgccacaccaacacactgaaccccgctcacgggcgccaggtcaccgtacaggagtttgcactcttcttcaccatattcgatgagactaagtcttggtatttcacagaaaatatggagcgaaactgccgggccccgtgcaacattcagatggaagacccaacgtttaaggagaactaccgctttcacgctattaatgggtacatcatggatacattgccaggcctggtgatggcccaggaccagaggattcgatggtacctgctgagcatgggtagcaatgaaaatattcacagcatccacttctccggtcacgtattcactgtgcgaaagaaagaagagtacaaaatggcgctttacaacctgtacccaggggtgtttgagacagtggagatgctgccaagtaaagccggcatctggagagtcgagtgcttgatcggcgagcatctccacgccggcatgtctactctgtttttagtatattcaaataaatgtcagacccccttagggatggcatctggccacattagggactttcaaattacagcctcaggtcagtacggtcagtgggcccctaaactggctcggcttcactattccggctcaattaatgcgtggtcaacaaaagagccgtttagttggataaaggttgacctgttagcccctatgatcatacatggcattaagacccagggggcacggcaaaagtttagctcactttacatcagtcaatttatcataatgtatagtttggacgggaaaaaatggcagacctaccggggaaattccaccgggacccttatggtattctttggtaatgttgattccagcggcatcaaacacaacatcttcaatcctccaatcatcgcccgctatattcggctgcatcctactcattacagcatccggtctacactgcggatggaactgatgggttgcgatctgaactcatgctccatgcctctcggcatggagtctaaagcaatttccgatgcgcagataactgccagctcatattttaccaacatgttcgctacttggagtccaagcaaggcacgcctgcatttacagggcaggtctaacgcctggcggcctcaggtgaataatccgaaagagtggcttcaggtggattttcagaaaacgatgaaggttactggtgtcacgacccaaggtgtgaagtcccttctgacgtccatgtatgtcaaggaattcttgatcagttcaagccaagacggacatcagtggaccctgttttttcaaaatggcaaggtgaaagtttttcaaggcaatcaggacagcttcacccctgttgttaacagccttgatccacccttactcacaagatacttgagaatccatccccagagttgggtgcaccaaatcgctctccggatggaggtgctgggttgtgaggcccaggacctgtactag

SEQ ID NO: 386 - protein, 1463 aa, Synthetic construct
  source 1..1463: note = Truncated FVIII v19
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 387 - DNA, 4392 bp, Synthetic construct
  source 1..4392: mol_type = other DNA; note = Gene codifying FVIII v20
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcttctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggatctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcttatgtgaaggtggatagctgccctgaggagccccaactgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcaactggaggaccctgagttccaggccagcaacatcatgcacagcatcaatggctatgtgtttgacagcctgcaactgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctctagctgtgacaagaacactggggactactatgaggacagctatgaggacatctctgcctacctgctgagcaagaacaatgccattgagcccaggagcttcagccagaacagcaggcaccccagcaccaggcagaagcagttcaacgccaccaccatccccgagaacaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcttgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagccctttagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaatagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagtctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactag

SEQ ID NO: 388 - DNA, 4392 bp, Synthetic construct
  source 1..4392: mol_type = other DNA; note = Gene codifying FVIII v21
atgcagatcgagctgtccacctgcttcttcctgtgcctgctgcggttctgcttttctgccacacggagatactatctgggagccgtggagctgtcttgggattacatgcagagcgacctgggagagctgccagtggacgcaaggtttccacctagagtgcctaagtccttccccttcaacacctctgtggtgtataagaaaaccctgttcgtggagtttacagatcacctgttcaacatcgccaagccaaggccaccatggatgggcctgctgggaccaacaatccaggcagaggtgtacgacaccgtggtcatcaccctgaagaacatggccagccaccccgtgagcctgcacgcagtgggcgtgtcctactggaaggccagcgagggagccgagtatgacgatcagacctctcagcgcgagaaggaggacgataaggtgtttccaggcggcagccacacatacgtgtggcaggtgctgaaggagaatggccccatggccagcgatcctctgtgcctgacctacagctatctgtcccacgtggatctggtgaaggatctgaacagcggcctgatcggggccctgctggtgtgcagagagggctccctggccaaggagaaaacccagacactgcacaagttcatcctgctgttcgccgtgtttgacgagggcaagtcttggcacagcgagacaaagaattctctgatgcaggacagggatgccgcaagcgccagggcatggccaaagatgcacacagtgaacggctacgtgaataggtccctgcccggcctgatcggctgtcacagaaagagcgtgtactggcacgtgatcggcatgggcaccacaccagaggtgcactccatcttcctggagggccacacctttctggtgaggaaccacaggcaggccagcctggagatcagccccatcaccttcctgacagcccagaccctgctgatggatctgggccagttcctgctgttttgccacatcagctcccaccagcacgatggcatggaagcctacgtgaaggtggactcctgccccgaggagccccagctcagaatgaagaacaatgaggaggccgaggactatgacgatgacctgaccgactccgagatggatgtggtgcggttcgatgacgataacagcccatcctttatccagatcagaagcgtggccaagaagcaccccaagacatgggtgcactacatcgccgccgaggaggaggactgggattatgccccactggtgctggcccccgacgatagatcctacaagtctcagtatctgaacaatggcccccagcggatcggcagaaagtacaagaaggtgaggttcatggcctatacagatgagacattcaagacacgcgaggccatccagcacgagagcggcatcctgggacctctgctgtacggagaagtgggcgacaccctgctgatcatctttaagaaccaggccagccggccctacaatatctatccacacggcatcacagatgtgcgccctctgtacagccggcggctgcccaagggagtgaagcacctgaaggacttcccaatcctgcccggcgagatctttaagtataagtggaccgtgacagtggaggatggccccaccaagtccgaccctaggtgcctgacacgctactattctagcttcgtgaatatggagagggatctggcaagcggcctgatcggacctctgctgatctgttacaaggagagcgtggatcagcggggcaaccagatcatgtccgacaagagaaatgtgatcctgttcagcgtgttcgacgagaaccggagctggtatctgaccgagaacatccagagattcctgcctaatccagccggcgtgcagctcgaggatccagagtttcaggccagcaacatcatgcactccatcaatggctacgtgttcgacagcctgcagctcagcgtgtgcctgcacgaggtggcctactggtatatcctgtccatcggagcccagaccgatttcctgagcgtgttcttttccggctacaccttcaagcacaagatggtgtatgaggacaccctgacactgttccctttttccggcgagacagtgttcatgtctatggagaatcccggcctgtggatcctgggctgccacaacagcgacttcagaaacagaggcatgaccgccctgctgaaggtgtcctcttgtgacaagaacacaggcgactactatgaggattcttacgaggacatcagcgcctatctgctgtccaagaacaatgccatcgagcctcgctcttttagccagaattctcggcacccaagcaccagacagaagcagttcaacgccaccacaatccccgagaataccacactgcagtctgatcaggaggagatcgactacgacgatacaatcagcgtggagatgaagaaggaggacttcgacatctacgacgaggatgagaaccagagccccaggtccttccagaagaaaacccgccactactttatcgccgccgtggagagactgtgggattatggcatgtcctctagcccccacgtgctgaggaatcgcgcccagtccggctctgtgcctcagttcaagaaggtggtgttccaggagtttaccgacggctcctttacacagcctctgtacaggggagagctgaacgagcacctgggcctgctgggaccatatatcagagccgaggtggaggataacatcatggtgaccttcaggaatcaggccagccggccctatagcttttattcctctctgatcagctacgaggaggaccagaggcagggagccgagccaagaaagaacttcgtgaagcccaatgagacaaagacctacttttggaaggtgcagcaccacatggcccccaccaaggacgagttcgattgcaaagcctgggcctatttttctgacgtggatctggagaaggacgtgcacagcggcctgatcggaccactgctggtgtgccacaccaacacactgaatccagcacacggccggcaggtcaccgtgcaggagttcgccctgttctttaccatctttgatgagacaaagagctggtacttcacagagaacatggagaggaattgccgcgccccttgtaatatccagatggaggacccaaccttcaaggagaactacagatttcacgccatcaatggctatatcatggatacactgcccggcctggtcatggcacaggaccagcggatcagatggtatctgctgtctatgggcagcaacgagaatatccactccatccacttctctggccacgtgtttaccgtgaggaagaaggaggagtataagatggccctgtacaacctgtatcccggcgtgttcgagacagtggagatgctgcctagcaaggcaggaatctggagggtggagtgcctgatcggagagcacctgcacgcaggcatgtctaccctgtttctggtgtacagcaataagtgtcagacacctctgggaatggcaagcggccacatcagggatttccagatcaccgcatccggccagtacggacagtgggccccaaagctggccagactgcactatagcggctccatcaacgcctggagcaccaaggagcctttctcctggatcaaggtggacctgctggccccaatgatcatccacggcatcaagacacagggagcccggcagaagttcagctccctgtacatctcccagtttatcatcatgtattctctggatggcaagaagtggcagacctacagaggcaatagcaccggcacactgatggtgttctttggcaacgtggactctagcggcatcaagcacaacatcttcaatccccctatcatcgcccggtacatcagactgcaccccacccactattccatcaggtctacactgcgcatggagctgatgggctgcgatctgaactcttgtagcatgcctctgggcatggagagcaaggccatctccgacgcccagatcaccgcctcctcttacttcaccaatatgtttgccacatggtccccctctaaggccaggctgcacctgcagggccggtccaacgcatggcggccccaggtgaacaatcccaaggagtggctgcaggtggattttcagaaaaccatgaaggtgacaggcgtgaccacacagggcgtgaagtctctgctgaccagcatgtatgtgaaggagttcctgatcagctcctctcaggacggccaccagtggacactgttctttcagaacggcaaggtgaaggtgttccagggcaatcaggatagctttaccccagtggtgaactccctggacccaccactgctgaccagatacctgcggatccaccctcagagctgggtgcaccagatcgccctgcgcatggaggtgctgggatgcgaggcacaggacctgtattag

SEQ ID NO: 389 - DNA, 4392 bp, Synthetic construct
  source 1..4392: mol_type = other DNA; note = Gene codifying FVIII v22
atgcagatagaactgagcacgtgcttttttttatgtctgctgcgattttgcttcagcgctacccgtcgatattacctgggggctgttgaattgtcttgggattacatgcagtcagatttaggcgaactccctgtggacgcccggttccccccccgcgtcccaaaaagcttcccttttaacacttccgttgtgtataagaagactttatttgtcgagtttacagaccacctctttaatattgccaagcccaggccaccctggatgggactgctcgggcctaccatccaggctgaggtgtatgacacggtggtgatcaccctcaaaaacatggccagccacccggtgtcactccacgctgtcggagtgtcttactggaaggcctccgaaggggccgagtacgatgaccagacctctcagagggagaaagaggacgataaggttttccccggcgggtcccacacttatgtgtggcaggttctgaaggagaacggcccaatggcgtccgatccattgtgtttgacttactcctacttgtctcacgtcgatctggttaaagacttaaactcaggcctgataggggcccttctcgtgtgtagagaaggtagcctcgctaaagagaagactcagaccctgcacaagtttattctgctctttgccgtgtttgatgagggtaagagctggcactccgaaacgaaaaattccctcatgcaggaccgggacgccgcttcagctcgggcctggcctaagatgcacacagtgaacgggtacgtgaatcgcagcctgcctggtttgatcggctgccatcgaaagagcgtgtattggcacgtgatcggaatgggcacaacccctgaggttcatagcatcttcttggagggccatacattcctggttaggaatcataggcaggcttctttggagatcagccccatcacctttctgacagcacaaacactgctgatggacctcggacaatttttgctgttttgtcacatctctagtcaccaacacgacgggatggaagcctatgtcaaagtggactcctgccccgaagaaccccagctccggatgaagaataatgaagaagccgaagactatgatgacgacttgaccgactccgagatggacgtggtgaggtttgacgatgataactctccttcctttatacagatcaggagtgtggctaaaaagcatccgaagacctgggtgcactatatcgccgcagaggaggaagattgggactatgccccactcgtgctggctcctgacgaccgcagttacaaatctcagtacctcaataacggcccccagaggattggtcgaaaatataagaaggtcagatttatggcctatacggacgagacattcaaaacaagggaagccatccagcacgaatccggcatcttgggcccactcctgtacggagaagtcggtgacaccctcctgattatttttaagaaccaggccagccgcccttacaatatatacccccatggcatcaccgatgttaggcctctgtattccaggcgcctgcccaaaggggtaaagcaccttaaagattttcccatcctgcccggcgaaatctttaagtataagtggaccgtgactgttgaagatgggcccactaaatcagatccaaggtgcctgactcggtattacagcagtttcgtcaatatggagagggatctcgcatcaggcctgatcggacctctgctgatctgctataaggagtccgtagatcagaggggaaatcagattatgagcgataagagaaatgtgattctgtttagtgtgtttgatgaaaaccgctcatggtacctgacggagaatatccagcgcttcctcccaaatcccgcaggcgtccagctcgaagaccccgagttccaggccagtaatatcatgcacagcatcaacgggtatgtttttgactccttgcagttgtccgtctgtctgcacgaggtcgcctactggtatattcttagcatcggagctcagacagatttcctcagcgttttctttagtgggtatacatttaagcacaaaatggtatacgaggatactctcaccttgttccctttctcaggcgagactgtctttatgtctatggaaaatcccggcctctggattttagggtgtcataattctgatttccgcaacagaggcatgacagccctgttaaaggtttcctcctgcgataagaatacaggcgattattatgaggattcttacgaggatattagtgcgtatttgctgagtaagaacaacgccatcgagccaaggagcttctcccagaactctagacacccctccaccagacagaagcaattcaatgctaccacaatccctgagaacaccacgctgcaatcagatcaggaggagattgattacgacgatactattagcgtggaaatgaagaaagaagatttcgatatatacgacgaggatgaaaaccagtcccctcgcagcttccagaaaaagaccaggcactattttatcgctgccgtggagagactgtgggattacggtatgagtagtagcccacatgtgttgcgaaaccgcgcacagtctggatctgttccccagtttaaaaaggtggtgtttcaagagttcaccgacgggtcttttacccagcctctgtaccgaggtgaactcaacgagcacctcgggctcctgggcccatatattcgagctgaggtagaggataacatcatggtgacatttaggaaccaagcctctcgaccatactccttctacagctcactcatcagctatgaggaggaccaacggcaaggtgccgaaccccggaagaatttcgtgaagcctaatgaaaccaagacatacttctggaaagttcaacatcatatggctcctactaaagacgaatttgattgtaaggcctgggcgtactttagcgacgtcgatctggaaaaggacgtgcatagcgggctcattggtccgcttttggtttgccacaccaacacactgaaccccgctcacgggcgccaggtcaccgtacaggagtttgcactcttcttcaccatattcgatgagactaagtcttggtatttcacagaaaatatggagcgaaactgccgggccccgtgcaacattcagatggaagacccaacgtttaaggagaactaccgctttcacgctattaatgggtacatcatggatacattgccaggcctggtgatggcccaggaccagaggattcgatggtacctgctgagcatgggtagcaatgaaaatattcacagcatccacttctccggtcacgtattcactgtgcgaaagaaagaagagtacaaaatggcgctttacaacctgtacccaggggtgtttgagacagtggagatgctgccaagtaaagccggcatctggagagtcgagtgcttgatcggcgagcatctccacgccggcatgtctactctgtttttagtatattcaaataaatgtcagacccccttagggatggcatctggccacattagggactttcaaattacagcctcaggtcagtacggtcagtgggcccctaaactggctcggcttcactattccggctcaattaatgcgtggtcaacaaaagagccgtttagttggataaaggttgacctgttagcccctatgatcatacatggcattaagacccagggggcacggcaaaagtttagctcactttacatcagtcaatttatcataatgtatagtttggacgggaaaaaatggcagacctaccggggaaattccaccgggacccttatggtattctttggtaatgttgattccagcggcatcaaacacaacatcttcaatcctccaatcatcgcccgctatattcggctgcatcctactcattacagcatccggtctacactgcggatggaactgatgggttgcgatctgaactcatgctccatgcctctcggcatggagtctaaagcaatttccgatgcgcagataactgccagctcatattttaccaacatgttcgctacttggagtccaagcaaggcacgcctgcatttacagggcaggtctaacgcctggcggcctcaggtgaataatccgaaagagtggcttcaggtggattttcagaaaacgatgaaggttactggtgtcacgacccaaggtgtgaagtcccttctgacgtccatgtatgtcaaggaattcttgatcagttcaagccaagacggacatcagtggaccctgttttttcaaaatggcaaggtgaaagtttttcaaggcaatcaggacagcttcacccctgttgttaacagccttgatccacccttactcacaagatacttgagaatccatccccagagttgggtgcaccaaatcgctctccggatggaggtgctgggttgtgaggcccaggacctgtactag

SEQ ID NO: 390 - protein, 1443 aa, Synthetic construct
  source 1..1443: note = Truncated FVIII v23
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 391 - DNA, 4332 bp, Synthetic construct
  source 1..4332: mol_type = other DNA; note = Gene codifying FVIII v24
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcttctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggatctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcttatgtgaaggtggatagctgccctgaggagccccaactgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcaactggaggaccctgagttccaggccagcaacatcatgcacagcatcaatggctatgtgtttgacagcctgcaactgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctctagctgtgacaagaacactggggactactatgaggacagctatgaggacatctctgcctacctgctgagcaagaacaatgccattgagcccagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcttgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagccctttagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaatagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagtctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactag

SEQ ID NO: 392 - DNA, 4332 bp, Synthetic construct
  source 1..4332: mol_type = other DNA; note = Gene codifying FVIII v25
atgcagatcgagctgtccacctgcttcttcctgtgcctgctgcggttctgcttttctgccacacggagatactatctgggagccgtggagctgtcttgggattacatgcagagcgacctgggagagctgccagtggacgcaaggtttccacctagagtgcctaagtccttccccttcaacacctctgtggtgtataagaaaaccctgttcgtggagtttacagatcacctgttcaacatcgccaagccaaggccaccatggatgggcctgctgggaccaacaatccaggcagaggtgtacgacaccgtggtcatcaccctgaagaacatggccagccaccccgtgagcctgcacgcagtgggcgtgtcctactggaaggccagcgagggagccgagtatgacgatcagacctctcagcgcgagaaggaggacgataaggtgtttccaggcggcagccacacatacgtgtggcaggtgctgaaggagaatggccccatggccagcgatcctctgtgcctgacctacagctatctgtcccacgtggatctggtgaaggatctgaacagcggcctgatcggggccctgctggtgtgcagagagggctccctggccaaggagaaaacccagacactgcacaagttcatcctgctgttcgccgtgtttgacgagggcaagtcttggcacagcgagacaaagaattctctgatgcaggacagggatgccgcaagcgccagggcatggccaaagatgcacacagtgaacggctacgtgaataggtccctgcccggcctgatcggctgtcacagaaagagcgtgtactggcacgtgatcggcatgggcaccacaccagaggtgcactccatcttcctggagggccacacctttctggtgaggaaccacaggcaggccagcctggagatcagccccatcaccttcctgacagcccagaccctgctgatggatctgggccagttcctgctgttttgccacatcagctcccaccagcacgatggcatggaagcctacgtgaaggtggactcctgccccgaggagccccagctcagaatgaagaacaatgaggaggccgaggactatgacgatgacctgaccgactccgagatggatgtggtgcggttcgatgacgataacagcccatcctttatccagatcagaagcgtggccaagaagcaccccaagacatgggtgcactacatcgccgccgaggaggaggactgggattatgccccactggtgctggcccccgacgatagatcctacaagtctcagtatctgaacaatggcccccagcggatcggcagaaagtacaagaaggtgaggttcatggcctatacagatgagacattcaagacacgcgaggccatccagcacgagagcggcatcctgggacctctgctgtacggagaagtgggcgacaccctgctgatcatctttaagaaccaggccagccggccctacaatatctatccacacggcatcacagatgtgcgccctctgtacagccggcggctgcccaagggagtgaagcacctgaaggacttcccaatcctgcccggcgagatctttaagtataagtggaccgtgacagtggaggatggccccaccaagtccgaccctaggtgcctgacacgctactattctagcttcgtgaatatggagagggatctggcaagcggcctgatcggacctctgctgatctgttacaaggagagcgtggatcagcggggcaaccagatcatgtccgacaagagaaatgtgatcctgttcagcgtgttcgacgagaaccggagctggtatctgaccgagaacatccagagattcctgcctaatccagccggcgtgcagctcgaggatccagagtttcaggccagcaacatcatgcactccatcaatggctacgtgttcgacagcctgcagctcagcgtgtgcctgcacgaggtggcctactggtatatcctgtccatcggagcccagaccgatttcctgagcgtgttcttttccggctacaccttcaagcacaagatggtgtatgaggacaccctgacactgttccctttttccggcgagacagtgttcatgtctatggagaatcccggcctgtggatcctgggctgccacaacagcgacttcagaaacagaggcatgaccgccctgctgaaggtgtcctcttgtgacaagaacacaggcgactactatgaggattcttacgaggacatcagcgcctatctgctgtccaagaacaatgccatcgagccacgcgagatcaccagaaccacactgcagtctgatcaggaggagatcgactacgacgatacaatcagcgtggagatgaagaaggaggacttcgacatctacgacgaggatgagaaccagagccccaggtccttccagaagaaaacccgccactactttatcgccgccgtggagagactgtgggattatggcatgtcctctagcccccacgtgctgaggaatcgcgcccagtccggctctgtgcctcagttcaagaaggtggtgttccaggagtttaccgacggctcctttacacagcctctgtacaggggagagctgaacgagcacctgggcctgctgggaccatatatcagagccgaggtggaggataacatcatggtgaccttcaggaatcaggccagccggccctatagcttttattcctctctgatcagctacgaggaggaccagaggcagggagccgagccaagaaagaacttcgtgaagcccaatgagacaaagacctacttttggaaggtgcagcaccacatggcccccaccaaggacgagttcgattgcaaagcctgggcctatttttctgacgtggatctggagaaggacgtgcacagcggcctgatcggaccactgctggtgtgccacaccaacacactgaatccagcacacggccggcaggtcaccgtgcaggagttcgccctgttctttaccatctttgatgagacaaagagctggtacttcacagagaacatggagaggaattgccgcgccccttgtaatatccagatggaggacccaaccttcaaggagaactacagatttcacgccatcaatggctatatcatggatacactgcccggcctggtcatggcacaggaccagcggatcagatggtatctgctgtctatgggcagcaacgagaatatccactccatccacttctctggccacgtgtttaccgtgaggaagaaggaggagtataagatggccctgtacaacctgtatcccggcgtgttcgagacagtggagatgctgcctagcaaggcaggaatctggagggtggagtgcctgatcggagagcacctgcacgcaggcatgtctaccctgtttctggtgtacagcaataagtgtcagacacctctgggaatggcaagcggccacatcagggatttccagatcaccgcatccggccagtacggacagtgggccccaaagctggccagactgcactatagcggctccatcaacgcctggagcaccaaggagcctttctcctggatcaaggtggacctgctggccccaatgatcatccacggcatcaagacacagggagcccggcagaagttcagctccctgtacatctcccagtttatcatcatgtattctctggatggcaagaagtggcagacctacagaggcaatagcaccggcacactgatggtgttctttggcaacgtggactctagcggcatcaagcacaacatcttcaatccccctatcatcgcccggtacatcagactgcaccccacccactattccatcaggtctacactgcgcatggagctgatgggctgcgatctgaactcttgtagcatgcctctgggcatggagagcaaggccatctccgacgcccagatcaccgcctcctcttacttcaccaatatgtttgccacatggtccccctctaaggccaggctgcacctgcagggccggtccaacgcatggcggccccaggtgaacaatcccaaggagtggctgcaggtggattttcagaaaaccatgaaggtgacaggcgtgaccacacagggcgtgaagtctctgctgaccagcatgtatgtgaaggagttcctgatcagctcctctcaggacggccaccagtggacactgttctttcagaacggcaaggtgaaggtgttccagggcaatcaggatagctttaccccagtggtgaactccctggacccaccactgctgaccagatacctgcggatccaccctcagagctgggtgcaccagatcgccctgcgcatggaggtgctgggatgcgaggcacaggacctgtattag

SEQ ID NO: 393 - DNA, 4332 bp, Synthetic construct
  source 1..4332: mol_type = other DNA; note = Gene codifying FVIII v26
atgcagatagaactgagcacgtgcttttttttatgtctgctgcgattttgcttcagcgctacccgtcgatattacctgggggctgttgaattgtcttgggattacatgcagtcagatttaggcgaactccctgtggacgcccggttccccccccgcgtcccaaaaagcttcccttttaacacttccgttgtgtataagaagactttatttgtcgagtttacagaccacctctttaatattgccaagcccaggccaccctggatgggactgctcgggcctaccatccaggctgaggtgtatgacacggtggtgatcaccctcaaaaacatggccagccacccggtgtcactccacgctgtcggagtgtcttactggaaggcctccgaaggggccgagtacgatgaccagacctctcagagggagaaagaggacgataaggttttccccggcgggtcccacacttatgtgtggcaggttctgaaggagaacggcccaatggcgtccgatccattgtgtttgacttactcctacttgtctcacgtcgatctggttaaagacttaaactcaggcctgataggggcccttctcgtgtgtagagaaggtagcctcgctaaagagaagactcagaccctgcacaagtttattctgctctttgccgtgtttgatgagggtaagagctggcactccgaaacgaaaaattccctcatgcaggaccgggacgccgcttcagctcgggcctggcctaagatgcacacagtgaacgggtacgtgaatcgcagcctgcctggtttgatcggctgccatcgaaagagcgtgtattggcacgtgatcggaatgggcacaacccctgaggttcatagcatcttcttggagggccatacattcctggttaggaatcataggcaggcttctttggagatcagccccatcacctttctgacagcacaaacactgctgatggacctcggacaatttttgctgttttgtcacatctctagtcaccaacacgacgggatggaagcctatgtcaaagtggactcctgccccgaagaaccccagctccggatgaagaataatgaagaagccgaagactatgatgacgacttgaccgactccgagatggacgtggtgaggtttgacgatgataactctccttcctttatacagatcaggagtgtggctaaaaagcatccgaagacctgggtgcactatatcgccgcagaggaggaagattgggactatgccccactcgtgctggctcctgacgaccgcagttacaaatctcagtacctcaataacggcccccagaggattggtcgaaaatataagaaggtcagatttatggcctatacggacgagacattcaaaacaagggaagccatccagcacgaatccggcatcttgggcccactcctgtacggagaagtcggtgacaccctcctgattatttttaagaaccaggccagccgcccttacaatatatacccccatggcatcaccgatgttaggcctctgtattccaggcgcctgcccaaaggggtaaagcaccttaaagattttcccatcctgcccggcgaaatctttaagtataagtggaccgtgactgttgaagatgggcccactaaatcagatccaaggtgcctgactcggtattacagcagtttcgtcaatatggagagggatctcgcatcaggcctgatcggacctctgctgatctgctataaggagtccgtagatcagaggggaaatcagattatgagcgataagagaaatgtgattctgtttagtgtgtttgatgaaaaccgctcatggtacctgacggagaatatccagcgcttcctcccaaatcccgcaggcgtccagctcgaagaccccgagttccaggccagtaatatcatgcacagcatcaacgggtatgtttttgactccttgcagttgtccgtctgtctgcacgaggtcgcctactggtatattcttagcatcggagctcagacagatttcctcagcgttttctttagtgggtatacatttaagcacaaaatggtatacgaggatactctcaccttgttccctttctcaggcgagactgtctttatgtctatggaaaatcccggcctctggattttagggtgtcataattctgatttccgcaacagaggcatgacagccctgttaaaggtttcctcctgcgataagaatacaggcgattattatgaggattcttacgaggatattagtgcgtatttgctgagtaagaacaacgccatcgagccaagggagatcacacggaccacgctgcaatcagatcaggaggagattgattacgacgatactattagcgtggaaatgaagaaagaagatttcgatatatacgacgaggatgaaaaccagtcccctcgcagcttccagaaaaagaccaggcactattttatcgctgccgtggagagactgtgggattacggtatgagtagtagcccacatgtgttgcgaaaccgcgcacagtctggatctgttccccagtttaaaaaggtggtgtttcaagagttcaccgacgggtcttttacccagcctctgtaccgaggtgaactcaacgagcacctcgggctcctgggcccatatattcgagctgaggtagaggataacatcatggtgacatttaggaaccaagcctctcgaccatactccttctacagctcactcatcagctatgaggaggaccaacggcaaggtgccgaaccccggaagaatttcgtgaagcctaatgaaaccaagacatacttctggaaagttcaacatcatatggctcctactaaagacgaatttgattgtaaggcctgggcgtactttagcgacgtcgatctggaaaaggacgtgcatagcgggctcattggtccgcttttggtttgccacaccaacacactgaaccccgctcacgggcgccaggtcaccgtacaggagtttgcactcttcttcaccatattcgatgagactaagtcttggtatttcacagaaaatatggagcgaaactgccgggccccgtgcaacattcagatggaagacccaacgtttaaggagaactaccgctttcacgctattaatgggtacatcatggatacattgccaggcctggtgatggcccaggaccagaggattcgatggtacctgctgagcatgggtagcaatgaaaatattcacagcatccacttctccggtcacgtattcactgtgcgaaagaaagaagagtacaaaatggcgctttacaacctgtacccaggggtgtttgagacagtggagatgctgccaagtaaagccggcatctggagagtcgagtgcttgatcggcgagcatctccacgccggcatgtctactctgtttttagtatattcaaataaatgtcagacccccttagggatggcatctggccacattagggactttcaaattacagcctcaggtcagtacggtcagtgggcccctaaactggctcggcttcactattccggctcaattaatgcgtggtcaacaaaagagccgtttagttggataaaggttgacctgttagcccctatgatcatacatggcattaagacccagggggcacggcaaaagtttagctcactttacatcagtcaatttatcataatgtatagtttggacgggaaaaaatggcagacctaccggggaaattccaccgggacccttatggtattctttggtaatgttgattccagcggcatcaaacacaacatcttcaatcctccaatcatcgcccgctatattcggctgcatcctactcattacagcatccggtctacactgcggatggaactgatgggttgcgatctgaactcatgctccatgcctctcggcatggagtctaaagcaatttccgatgcgcagataactgccagctcatattttaccaacatgttcgctacttggagtccaagcaaggcacgcctgcatttacagggcaggtctaacgcctggcggcctcaggtgaataatccgaaagagtggcttcaggtggattttcagaaaacgatgaaggttactggtgtcacgacccaaggtgtgaagtcccttctgacgtccatgtatgtcaaggaattcttgatcagttcaagccaagacggacatcagtggaccctgttttttcaaaatggcaaggtgaaagtttttcaaggcaatcaggacagcttcacccctgttgttaacagccttgatccacccttactcacaagatacttgagaatccatccccagagttgggtgcaccaaatcgctctccggatggaggtgctgggttgtgaggcccaggacctgtactag

SEQ ID NO: 394 - protein, 1464 aa, Synthetic construct
  source 1..1464: note = Truncated FVIII v27
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 395 - DNA, 4395 bp, Synthetic construct
  source 1..4395: mol_type = other DNA; note = Gene codifying FVIII v28
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcttctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggatctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcttatgtgaaggtggatagctgccctgaggagccccaactgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcaactggaggaccctgagttccaggccagcaacatcatgcacagcatcaatggctatgtgtttgacagcctgcaactgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctctagctgtgacaagaacactggggactactatgaggacagctatgaggacatctctgcctacctgctgagcaagaacaatgccattgagcccaggagcttcagccagaacagcaggcaccccagccagaaccccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcttgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagccctttagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaatagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagtctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactag

SEQ ID NO: 396 - DNA, 4395 bp, Synthetic construct
  source 1..4395: mol_type = other DNA; note = Gene codifying FVIII v29
atgcagatcgagctgtccacctgcttcttcctgtgcctgctgcggttctgcttttctgccacacggagatactatctgggagccgtggagctgtcttgggattacatgcagagcgacctgggagagctgccagtggacgcaaggtttccacctagagtgcctaagtccttccccttcaacacctctgtggtgtataagaaaaccctgttcgtggagtttacagatcacctgttcaacatcgccaagccaaggccaccatggatgggcctgctgggaccaacaatccaggcagaggtgtacgacaccgtggtcatcaccctgaagaacatggccagccaccccgtgagcctgcacgcagtgggcgtgtcctactggaaggccagcgagggagccgagtatgacgatcagacctctcagcgcgagaaggaggacgataaggtgtttccaggcggcagccacacatacgtgtggcaggtgctgaaggagaatggccccatggccagcgatcctctgtgcctgacctacagctatctgtcccacgtggatctggtgaaggatctgaacagcggcctgatcggggccctgctggtgtgcagagagggctccctggccaaggagaaaacccagacactgcacaagttcatcctgctgttcgccgtgtttgacgagggcaagtcttggcacagcgagacaaagaattctctgatgcaggacagggatgccgcaagcgccagggcatggccaaagatgcacacagtgaacggctacgtgaataggtccctgcccggcctgatcggctgtcacagaaagagcgtgtactggcacgtgatcggcatgggcaccacaccagaggtgcactccatcttcctggagggccacacctttctggtgaggaaccacaggcaggccagcctggagatcagccccatcaccttcctgacagcccagaccctgctgatggatctgggccagttcctgctgttttgccacatcagctcccaccagcacgatggcatggaagcctacgtgaaggtggactcctgccccgaggagccccagctcagaatgaagaacaatgaggaggccgaggactatgacgatgacctgaccgactccgagatggatgtggtgcggttcgatgacgataacagcccatcctttatccagatcagaagcgtggccaagaagcaccccaagacatgggtgcactacatcgccgccgaggaggaggactgggattatgccccactggtgctggcccccgacgatagatcctacaagtctcagtatctgaacaatggcccccagcggatcggcagaaagtacaagaaggtgaggttcatggcctatacagatgagacattcaagacacgcgaggccatccagcacgagagcggcatcctgggacctctgctgtacggagaagtgggcgacaccctgctgatcatctttaagaaccaggccagccggccctacaatatctatccacacggcatcacagatgtgcgccctctgtacagccggcggctgcccaagggagtgaagcacctgaaggacttcccaatcctgcccggcgagatctttaagtataagtggaccgtgacagtggaggatggccccaccaagtccgaccctaggtgcctgacacgctactattctagcttcgtgaatatggagagggatctggcaagcggcctgatcggacctctgctgatctgttacaaggagagcgtggatcagcggggcaaccagatcatgtccgacaagagaaatgtgatcctgttcagcgtgttcgacgagaaccggagctggtatctgaccgagaacatccagagattcctgcctaatccagccggcgtgcagctcgaggatccagagtttcaggccagcaacatcatgcactccatcaatggctacgtgttcgacagcctgcagctcagcgtgtgcctgcacgaggtggcctactggtatatcctgtccatcggagcccagaccgatttcctgagcgtgttcttttccggctacaccttcaagcacaagatggtgtatgaggacaccctgacactgttccctttttccggcgagacagtgttcatgtctatggagaatcccggcctgtggatcctgggctgccacaacagcgacttcagaaacagaggcatgaccgccctgctgaaggtgtcctcttgtgacaagaacacaggcgactactatgaggattcttacgaggacatcagcgcctatctgctgtccaagaacaatgccatcgagcctcgctcttttagccagaattctcggcacccaagccagaaccctccagtgctgaagcggcaccagcgggagatcaccagaaccacactgcagtctgatcaggaggagatcgactacgacgatacaatcagcgtggagatgaagaaggaggacttcgacatctacgacgaggatgagaaccagagccccaggtccttccagaagaaaacccgccactactttatcgccgccgtggagagactgtgggattatggcatgtcctctagcccccacgtgctgaggaatcgcgcccagtccggctctgtgcctcagttcaagaaggtggtgttccaggagtttaccgacggctcctttacacagcctctgtacaggggagagctgaacgagcacctgggcctgctgggaccatatatcagagccgaggtggaggataacatcatggtgaccttcaggaatcaggccagccggccctatagcttttattcctctctgatcagctacgaggaggaccagaggcagggagccgagccaagaaagaacttcgtgaagcccaatgagacaaagacctacttttggaaggtgcagcaccacatggcccccaccaaggacgagttcgattgcaaagcctgggcctatttttctgacgtggatctggagaaggacgtgcacagcggcctgatcggaccactgctggtgtgccacaccaacacactgaatccagcacacggccggcaggtcaccgtgcaggagttcgccctgttctttaccatctttgatgagacaaagagctggtacttcacagagaacatggagaggaattgccgcgccccttgtaatatccagatggaggacccaaccttcaaggagaactacagatttcacgccatcaatggctatatcatggatacactgcccggcctggtcatggcacaggaccagcggatcagatggtatctgctgtctatgggcagcaacgagaatatccactccatccacttctctggccacgtgtttaccgtgaggaagaaggaggagtataagatggccctgtacaacctgtatcccggcgtgttcgagacagtggagatgctgcctagcaaggcaggaatctggagggtggagtgcctgatcggagagcacctgcacgcaggcatgtctaccctgtttctggtgtacagcaataagtgtcagacacctctgggaatggcaagcggccacatcagggatttccagatcaccgcatccggccagtacggacagtgggccccaaagctggccagactgcactatagcggctccatcaacgcctggagcaccaaggagcctttctcctggatcaaggtggacctgctggccccaatgatcatccacggcatcaagacacagggagcccggcagaagttcagctccctgtacatctcccagtttatcatcatgtattctctggatggcaagaagtggcagacctacagaggcaatagcaccggcacactgatggtgttctttggcaacgtggactctagcggcatcaagcacaacatcttcaatccccctatcatcgcccggtacatcagactgcaccccacccactattccatcaggtctacactgcgcatggagctgatgggctgcgatctgaactcttgtagcatgcctctgggcatggagagcaaggccatctccgacgcccagatcaccgcctcctcttacttcaccaatatgtttgccacatggtccccctctaaggccaggctgcacctgcagggccggtccaacgcatggcggccccaggtgaacaatcccaaggagtggctgcaggtggattttcagaaaaccatgaaggtgacaggcgtgaccacacagggcgtgaagtctctgctgaccagcatgtatgtgaaggagttcctgatcagctcctctcaggacggccaccagtggacactgttctttcagaacggcaaggtgaaggtgttccagggcaatcaggatagctttaccccagtggtgaactccctggacccaccactgctgaccagatacctgcggatccaccctcagagctgggtgcaccagatcgccctgcgcatggaggtgctgggatgcgaggcacaggacctgtattag

SEQ ID NO: 397 - DNA, 4395 bp, Synthetic construct
  source 1..4395: mol_type = other DNA; note = Gene codifying FVIII v30
atgcagatagaactgagcacgtgcttttttttatgtctgctgcgattttgcttcagcgctacccgtcgatattacctgggggctgttgaattgtcttgggattacatgcagtcagatttaggcgaactccctgtggacgcccggttccccccccgcgtcccaaaaagcttcccttttaacacttccgttgtgtataagaagactttatttgtcgagtttacagaccacctctttaatattgccaagcccaggccaccctggatgggactgctcgggcctaccatccaggctgaggtgtatgacacggtggtgatcaccctcaaaaacatggccagccacccggtgtcactccacgctgtcggagtgtcttactggaaggcctccgaaggggccgagtacgatgaccagacctctcagagggagaaagaggacgataaggttttccccggcgggtcccacacttatgtgtggcaggttctgaaggagaacggcccaatggcgtccgatccattgtgtttgacttactcctacttgtctcacgtcgatctggttaaagacttaaactcaggcctgataggggcccttctcgtgtgtagagaaggtagcctcgctaaagagaagactcagaccctgcacaagtttattctgctctttgccgtgtttgatgagggtaagagctggcactccgaaacgaaaaattccctcatgcaggaccgggacgccgcttcagctcgggcctggcctaagatgcacacagtgaacgggtacgtgaatcgcagcctgcctggtttgatcggctgccatcgaaagagcgtgtattggcacgtgatcggaatgggcacaacccctgaggttcatagcatcttcttggagggccatacattcctggttaggaatcataggcaggcttctttggagatcagccccatcacctttctgacagcacaaacactgctgatggacctcggacaatttttgctgttttgtcacatctctagtcaccaacacgacgggatggaagcctatgtcaaagtggactcctgccccgaagaaccccagctccggatgaagaataatgaagaagccgaagactatgatgacgacttgaccgactccgagatggacgtggtgaggtttgacgatgataactctccttcctttatacagatcaggagtgtggctaaaaagcatccgaagacctgggtgcactatatcgccgcagaggaggaagattgggactatgccccactcgtgctggctcctgacgaccgcagttacaaatctcagtacctcaataacggcccccagaggattggtcgaaaatataagaaggtcagatttatggcctatacggacgagacattcaaaacaagggaagccatccagcacgaatccggcatcttgggcccactcctgtacggagaagtcggtgacaccctcctgattatttttaagaaccaggccagccgcccttacaatatatacccccatggcatcaccgatgttaggcctctgtattccaggcgcctgcccaaaggggtaaagcaccttaaagattttcccatcctgcccggcgaaatctttaagtataagtggaccgtgactgttgaagatgggcccactaaatcagatccaaggtgcctgactcggtattacagcagtttcgtcaatatggagagggatctcgcatcaggcctgatcggacctctgctgatctgctataaggagtccgtagatcagaggggaaatcagattatgagcgataagagaaatgtgattctgtttagtgtgtttgatgaaaaccgctcatggtacctgacggagaatatccagcgcttcctcccaaatcccgcaggcgtccagctcgaagaccccgagttccaggccagtaatatcatgcacagcatcaacgggtatgtttttgactccttgcagttgtccgtctgtctgcacgaggtcgcctactggtatattcttagcatcggagctcagacagatttcctcagcgttttctttagtgggtatacatttaagcacaaaatggtatacgaggatactctcaccttgttccctttctcaggcgagactgtctttatgtctatggaaaatcccggcctctggattttagggtgtcataattctgatttccgcaacagaggcatgacagccctgttaaaggtttcctcctgcgataagaatacaggcgattattatgaggattcttacgaggatattagtgcgtatttgctgagtaagaacaacgccatcgagccaaggagcttctcccagaactctagacacccctcccagaacccccctgtgcttaagaggcatcagagagagatcacacggaccacgctgcaatcagatcaggaggagattgattacgacgatactattagcgtggaaatgaagaaagaagatttcgatatatacgacgaggatgaaaaccagtcccctcgcagcttccagaaaaagaccaggcactattttatcgctgccgtggagagactgtgggattacggtatgagtagtagcccacatgtgttgcgaaaccgcgcacagtctggatctgttccccagtttaaaaaggtggtgtttcaagagttcaccgacgggtcttttacccagcctctgtaccgaggtgaactcaacgagcacctcgggctcctgggcccatatattcgagctgaggtagaggataacatcatggtgacatttaggaaccaagcctctcgaccatactccttctacagctcactcatcagctatgaggaggaccaacggcaaggtgccgaaccccggaagaatttcgtgaagcctaatgaaaccaagacatacttctggaaagttcaacatcatatggctcctactaaagacgaatttgattgtaaggcctgggcgtactttagcgacgtcgatctggaaaaggacgtgcatagcgggctcattggtccgcttttggtttgccacaccaacacactgaaccccgctcacgggcgccaggtcaccgtacaggagtttgcactcttcttcaccatattcgatgagactaagtcttggtatttcacagaaaatatggagcgaaactgccgggccccgtgcaacattcagatggaagacccaacgtttaaggagaactaccgctttcacgctattaatgggtacatcatggatacattgccaggcctggtgatggcccaggaccagaggattcgatggtacctgctgagcatgggtagcaatgaaaatattcacagcatccacttctccggtcacgtattcactgtgcgaaagaaagaagagtacaaaatggcgctttacaacctgtacccaggggtgtttgagacagtggagatgctgccaagtaaagccggcatctggagagtcgagtgcttgatcggcgagcatctccacgccggcatgtctactctgtttttagtatattcaaataaatgtcagacccccttagggatggcatctggccacattagggactttcaaattacagcctcaggtcagtacggtcagtgggcccctaaactggctcggcttcactattccggctcaattaatgcgtggtcaacaaaagagccgtttagttggataaaggttgacctgttagcccctatgatcatacatggcattaagacccagggggcacggcaaaagtttagctcactttacatcagtcaatttatcataatgtatagtttggacgggaaaaaatggcagacctaccggggaaattccaccgggacccttatggtattctttggtaatgttgattccagcggcatcaaacacaacatcttcaatcctccaatcatcgcccgctatattcggctgcatcctactcattacagcatccggtctacactgcggatggaactgatgggttgcgatctgaactcatgctccatgcctctcggcatggagtctaaagcaatttccgatgcgcagataactgccagctcatattttaccaacatgttcgctacttggagtccaagcaaggcacgcctgcatttacagggcaggtctaacgcctggcggcctcaggtgaataatccgaaagagtggcttcaggtggattttcagaaaacgatgaaggttactggtgtcacgacccaaggtgtgaagtcccttctgacgtccatgtatgtcaaggaattcttgatcagttcaagccaagacggacatcagtggaccctgttttttcaaaatggcaaggtgaaagtttttcaaggcaatcaggacagcttcacccctgttgttaacagccttgatccacccttactcacaagatacttgagaatccatccccagagttgggtgcaccaaatcgctctccggatggaggtgctgggttgtgaggcccaggacctgtactag

SEQ ID NO: 398 - protein, 1670 aa, Synthetic construct
  source 1..1670: note = Truncated FVIII v31
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 399 - DNA, 5013 bp, Synthetic construct
  source 1..5013: mol_type = other DNA; note = Gene codifying FVIII v32
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcttctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggatctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcttatgtgaaggtggatagctgccctgaggagccccaactgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcaactggaggaccctgagttccaggccagcaacatcatgcacagcatcaatggctatgtgtttgacagcctgcaactgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctctagctgtgacaagaacactggggactactatgaggacagctatgaggacatctctgcctacctgctgagcaagaacaatgccattgagcccaggagcttcagccagaacagcaggcaccccagcaccaggcagaagcagttcaacgccaccaccatccccgagaacgacatcgagaagaccgacccctggttcgcccacaggacccccatgcccaagatccagaacgtgagcagcagcgacctgctgatgctgctgaggcagagccccaccccccacggcctgagcctgagcgacctgcaggaggccaagtacgaaaccttcagcgacgaccccagccccggcgccatcgacagcaacaacagcctgagcgagatgacccacttcaggccccaactgcaccacagcggcgacatggtgttcacccccgagagcggcctgcaactgaggctgaacgagaagctgggcaccaccgccgccaccgagctgaagaagctggacttcaaggtgagcagcaccagcaacaacctgatcagcaccatccccagcgacaacctggccgccggcaccgacaacaccagcagcctgggcccccccagcatgcccgtgcactacgacagccaactggacaccaccctgttcggcaagaagtctagccccctgaccgagagcggcggccccctgagcctgagcgaggagaacaacgacagcaagctgctggagagcggcctgatgaacagccaggagagctcttggggcaagaacgtgagcagcagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcttgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagccctttagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaatagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagtctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactag

SEQ ID NO: 400 - DNA, 5013 bp, Synthetic construct
  source 1..5013: mol_type = other DNA; note = Gene codifying FVIII v33
atgcagatcgagctgtccacctgcttcttcctgtgcctgctgcggttctgcttttctgccacacggagatactatctgggagccgtggagctgtcttgggattacatgcagagcgacctgggagagctgccagtggacgcaaggtttccacctagagtgcctaagtccttccccttcaacacctctgtggtgtataagaaaaccctgttcgtggagtttacagatcacctgttcaacatcgccaagccaaggccaccatggatgggcctgctgggaccaacaatccaggcagaggtgtacgacaccgtggtcatcaccctgaagaacatggccagccaccccgtgagcctgcacgcagtgggcgtgtcctactggaaggccagcgagggagccgagtatgacgatcagacctctcagcgcgagaaggaggacgataaggtgtttccaggcggcagccacacatacgtgtggcaggtgctgaaggagaatggccccatggccagcgatcctctgtgcctgacctacagctatctgtcccacgtggatctggtgaaggatctgaacagcggcctgatcggggccctgctggtgtgcagagagggctccctggccaaggagaaaacccagacactgcacaagttcatcctgctgttcgccgtgtttgacgagggcaagtcttggcacagcgagacaaagaattctctgatgcaggacagggatgccgcaagcgccagggcatggccaaagatgcacacagtgaacggctacgtgaataggtccctgcccggcctgatcggctgtcacagaaagagcgtgtactggcacgtgatcggcatgggcaccacaccagaggtgcactccatcttcctggagggccacacctttctggtgaggaaccacaggcaggccagcctggagatcagccccatcaccttcctgacagcccagaccctgctgatggatctgggccagttcctgctgttttgccacatcagctcccaccagcacgatggcatggaagcctacgtgaaggtggactcctgccccgaggagccccagctcagaatgaagaacaatgaggaggccgaggactatgacgatgacctgaccgactccgagatggatgtggtgcggttcgatgacgataacagcccatcctttatccagatcagaagcgtggccaagaagcaccccaagacatgggtgcactacatcgccgccgaggaggaggactgggattatgccccactggtgctggcccccgacgatagatcctacaagtctcagtatctgaacaatggcccccagcggatcggcagaaagtacaagaaggtgaggttcatggcctatacagatgagacattcaagacacgcgaggccatccagcacgagagcggcatcctgggacctctgctgtacggagaagtgggcgacaccctgctgatcatctttaagaaccaggccagccggccctacaatatctatccacacggcatcacagatgtgcgccctctgtacagccggcggctgcccaagggagtgaagcacctgaaggacttcccaatcctgcccggcgagatctttaagtataagtggaccgtgacagtggaggatggccccaccaagtccgaccctaggtgcctgacacgctactattctagcttcgtgaatatggagagggatctggcaagcggcctgatcggacctctgctgatctgttacaaggagagcgtggatcagcggggcaaccagatcatgtccgacaagagaaatgtgatcctgttcagcgtgttcgacgagaaccggagctggtatctgaccgagaacatccagagattcctgcctaatccagccggcgtgcagctcgaggatccagagtttcaggccagcaacatcatgcactccatcaatggctacgtgttcgacagcctgcagctcagcgtgtgcctgcacgaggtggcctactggtatatcctgtccatcggagcccagaccgatttcctgagcgtgttcttttccggctacaccttcaagcacaagatggtgtatgaggacaccctgacactgttccctttttccggcgagacagtgttcatgtctatggagaatcccggcctgtggatcctgggctgccacaacagcgacttcagaaacagaggcatgaccgccctgctgaaggtgtcctcttgtgacaagaacacaggcgactactatgaggattcttacgaggacatcagcgcctatctgctgtccaagaacaatgccatcgagcctcgctcttttagccagaattctcggcacccaagcaccagacagaagcagttcaacgccaccacaatccccgagaatgacatcgagaaaaccgacccttggtttgcccacaggacacccatgcctaagatccagaacgtgagctcctctgatctgctgatgctgctgaggcagtccccaaccccacacggcctgtccctgtctgacctgcaggaggccaagtacgagacattctccgacgatcccagccccggggccatcgacagcaacaatagcctgtccgagatgacccacttcaggccccagctccaccactccggcgacatggtgtttacacctgagagcggcctgcagctccgcctgaatgagaagctgggcaccacagccgccaccgagctgaagaagctggactttaaggtgagctccacctccaacaatctgatcagcacaatcccatccgataacctggccgccggcaccgacaatacatctagcctgggccctccaagcatgcccgtgcactacgattcccagctcgacaccacactgttcggcaagaagtcctctcccctgaccgagagcggcggacctctgtctctgagcgaggagaacaatgactctaagctgctggagagcggcctgatgaactcccaggagagctcctggggcaagaacgtgagcagccgggagatcaccagaaccacactgcagtctgatcaggaggagatcgactacgacgatacaatcagcgtggagatgaagaaggaggacttcgacatctacgacgaggatgagaaccagagccccaggtccttccagaagaaaacccgccactactttatcgccgccgtggagagactgtgggattatggcatgtcctctagcccccacgtgctgaggaatcgcgcccagtccggctctgtgcctcagttcaagaaggtggtgttccaggagtttaccgacggctcctttacacagcctctgtacaggggagagctgaacgagcacctgggcctgctgggaccatatatcagagccgaggtggaggataacatcatggtgaccttcaggaatcaggccagccggccctatagcttttattcctctctgatcagctacgaggaggaccagaggcagggagccgagccaagaaagaacttcgtgaagcccaatgagacaaagacctacttttggaaggtgcagcaccacatggcccccaccaaggacgagttcgattgcaaagcctgggcctatttttctgacgtggatctggagaaggacgtgcacagcggcctgatcggaccactgctggtgtgccacaccaacacactgaatccagcacacggccggcaggtcaccgtgcaggagttcgccctgttctttaccatctttgatgagacaaagagctggtacttcacagagaacatggagaggaattgccgcgccccttgtaatatccagatggaggacccaaccttcaaggagaactacagatttcacgccatcaatggctatatcatggatacactgcccggcctggtcatggcacaggaccagcggatcagatggtatctgctgtctatgggcagcaacgagaatatccactccatccacttctctggccacgtgtttaccgtgaggaagaaggaggagtataagatggccctgtacaacctgtatcccggcgtgttcgagacagtggagatgctgcctagcaaggcaggaatctggagggtggagtgcctgatcggagagcacctgcacgcaggcatgtctaccctgtttctggtgtacagcaataagtgtcagacacctctgggaatggcaagcggccacatcagggatttccagatcaccgcatccggccagtacggacagtgggccccaaagctggccagactgcactatagcggctccatcaacgcctggagcaccaaggagcctttctcctggatcaaggtggacctgctggccccaatgatcatccacggcatcaagacacagggagcccggcagaagttcagctccctgtacatctcccagtttatcatcatgtattctctggatggcaagaagtggcagacctacagaggcaatagcaccggcacactgatggtgttctttggcaacgtggactctagcggcatcaagcacaacatcttcaatccccctatcatcgcccggtacatcagactgcaccccacccactattccatcaggtctacactgcgcatggagctgatgggctgcgatctgaactcttgtagcatgcctctgggcatggagagcaaggccatctccgacgcccagatcaccgcctcctcttacttcaccaatatgtttgccacatggtccccctctaaggccaggctgcacctgcagggccggtccaacgcatggcggccccaggtgaacaatcccaaggagtggctgcaggtggattttcagaaaaccatgaaggtgacaggcgtgaccacacagggcgtgaagtctctgctgaccagcatgtatgtgaaggagttcctgatcagctcctctcaggacggccaccagtggacactgttctttcagaacggcaaggtgaaggtgttccagggcaatcaggatagctttaccccagtggtgaactccctggacccaccactgctgaccagatacctgcggatccaccctcagagctgggtgcaccagatcgccctgcgcatggaggtgctgggatgcgaggcacaggacctgtattag

SEQ ID NO: 401 - DNA, 5013 bp, Synthetic construct
  source 1..5013: mol_type = other DNA; note = Gene codifying FVIII v34
atgcagatagaactgagcacgtgcttttttttatgtctgctgcgattttgcttcagcgctacccgtcgatattacctgggggctgttgaattgtcttgggattacatgcagtcagatttaggcgaactccctgtggacgcccggttccccccccgcgtcccaaaaagcttcccttttaacacttccgttgtgtataagaagactttatttgtcgagtttacagaccacctctttaatattgccaagcccaggccaccctggatgggactgctcgggcctaccatccaggctgaggtgtatgacacggtggtgatcaccctcaaaaacatggccagccacccggtgtcactccacgctgtcggagtgtcttactggaaggcctccgaaggggccgagtacgatgaccagacctctcagagggagaaagaggacgataaggttttccccggcgggtcccacacttatgtgtggcaggttctgaaggagaacggcccaatggcgtccgatccattgtgtttgacttactcctacttgtctcacgtcgatctggttaaagacttaaactcaggcctgataggggcccttctcgtgtgtagagaaggtagcctcgctaaagagaagactcagaccctgcacaagtttattctgctctttgccgtgtttgatgagggtaagagctggcactccgaaacgaaaaattccctcatgcaggaccgggacgccgcttcagctcgggcctggcctaagatgcacacagtgaacgggtacgtgaatcgcagcctgcctggtttgatcggctgccatcgaaagagcgtgtattggcacgtgatcggaatgggcacaacccctgaggttcatagcatcttcttggagggccatacattcctggttaggaatcataggcaggcttctttggagatcagccccatcacctttctgacagcacaaacactgctgatggacctcggacaatttttgctgttttgtcacatctctagtcaccaacacgacgggatggaagcctatgtcaaagtggactcctgccccgaagaaccccagctccggatgaagaataatgaagaagccgaagactatgatgacgacttgaccgactccgagatggacgtggtgaggtttgacgatgataactctccttcctttatacagatcaggagtgtggctaaaaagcatccgaagacctgggtgcactatatcgccgcagaggaggaagattgggactatgccccactcgtgctggctcctgacgaccgcagttacaaatctcagtacctcaataacggcccccagaggattggtcgaaaatataagaaggtcagatttatggcctatacggacgagacattcaaaacaagggaagccatccagcacgaatccggcatcttgggcccactcctgtacggagaagtcggtgacaccctcctgattatttttaagaaccaggccagccgcccttacaatatatacccccatggcatcaccgatgttaggcctctgtattccaggcgcctgcccaaaggggtaaagcaccttaaagattttcccatcctgcccggcgaaatctttaagtataagtggaccgtgactgttgaagatgggcccactaaatcagatccaaggtgcctgactcggtattacagcagtttcgtcaatatggagagggatctcgcatcaggcctgatcggacctctgctgatctgctataaggagtccgtagatcagaggggaaatcagattatgagcgataagagaaatgtgattctgtttagtgtgtttgatgaaaaccgctcatggtacctgacggagaatatccagcgcttcctcccaaatcccgcaggcgtccagctcgaagaccccgagttccaggccagtaatatcatgcacagcatcaacgggtatgtttttgactccttgcagttgtccgtctgtctgcacgaggtcgcctactggtatattcttagcatcggagctcagacagatttcctcagcgttttctttagtgggtatacatttaagcacaaaatggtatacgaggatactctcaccttgttccctttctcaggcgagactgtctttatgtctatggaaaatcccggcctctggattttagggtgtcataattctgatttccgcaacagaggcatgacagccctgttaaaggtttcctcctgcgataagaatacaggcgattattatgaggattcttacgaggatattagtgcgtatttgctgagtaagaacaacgccatcgagccaaggagcttctcccagaactctagacacccctccaccagacagaagcaattcaatgctaccacaatccctgagaacgacatcgaaaagactgacccctggtttgcgcacaggactcccatgcccaagattcaaaatgtgtccagttctgatctgctgatgctgctgaggcagtctccaactccccacgggcttagcctgtctgacctgcaggaagccaagtatgagacatttagcgatgatccctcccctggcgctattgatagtaataacagcctctccgagatgactcacttcaggccccagttacaccattccggcgacatggtcttcacgccagaatccgggcttcaactgcgcctcaacgaaaagctcgggacaaccgccgcaaccgagcttaaaaagctcgacttcaaagtctctagcaccagcaataacctgattagtacaatccctagtgacaatttggccgcgggaacagacaacacctcttccctgggccctccatcaatgcctgtccactacgattcccagctcgataccactctttttggaaaaaaatcttccccactgactgagtccggcggccctctgtccttatctgaggaaaacaacgacagcaagctcctggaaagcgggcttatgaacagtcaagagtcttcttggggtaagaacgtgtctagtagagagatcacacggaccacgctgcaatcagatcaggaggagattgattacgacgatactattagcgtggaaatgaagaaagaagatttcgatatatacgacgaggatgaaaaccagtcccctcgcagcttccagaaaaagaccaggcactattttatcgctgccgtggagagactgtgggattacggtatgagtagtagcccacatgtgttgcgaaaccgcgcacagtctggatctgttccccagtttaaaaaggtggtgtttcaagagttcaccgacgggtcttttacccagcctctgtaccgaggtgaactcaacgagcacctcgggctcctgggcccatatattcgagctgaggtagaggataacatcatggtgacatttaggaaccaagcctctcgaccatactccttctacagctcactcatcagctatgaggaggaccaacggcaaggtgccgaaccccggaagaatttcgtgaagcctaatgaaaccaagacatacttctggaaagttcaacatcatatggctcctactaaagacgaatttgattgtaaggcctgggcgtactttagcgacgtcgatctggaaaaggacgtgcatagcgggctcattggtccgcttttggtttgccacaccaacacactgaaccccgctcacgggcgccaggtcaccgtacaggagtttgcactcttcttcaccatattcgatgagactaagtcttggtatttcacagaaaatatggagcgaaactgccgggccccgtgcaacattcagatggaagacccaacgtttaaggagaactaccgctttcacgctattaatgggtacatcatggatacattgccaggcctggtgatggcccaggaccagaggattcgatggtacctgctgagcatgggtagcaatgaaaatattcacagcatccacttctccggtcacgtattcactgtgcgaaagaaagaagagtacaaaatggcgctttacaacctgtacccaggggtgtttgagacagtggagatgctgccaagtaaagccggcatctggagagtcgagtgcttgatcggcgagcatctccacgccggcatgtctactctgtttttagtatattcaaataaatgtcagacccccttagggatggcatctggccacattagggactttcaaattacagcctcaggtcagtacggtcagtgggcccctaaactggctcggcttcactattccggctcaattaatgcgtggtcaacaaaagagccgtttagttggataaaggttgacctgttagcccctatgatcatacatggcattaagacccagggggcacggcaaaagtttagctcactttacatcagtcaatttatcataatgtatagtttggacgggaaaaaatggcagacctaccggggaaattccaccgggacccttatggtattctttggtaatgttgattccagcggcatcaaacacaacatcttcaatcctccaatcatcgcccgctatattcggctgcatcctactcattacagcatccggtctacactgcggatggaactgatgggttgcgatctgaactcatgctccatgcctctcggcatggagtctaaagcaatttccgatgcgcagataactgccagctcatattttaccaacatgttcgctacttggagtccaagcaaggcacgcctgcatttacagggcaggtctaacgcctggcggcctcaggtgaataatccgaaagagtggcttcaggtggattttcagaaaacgatgaaggttactggtgtcacgacccaaggtgtgaagtcccttctgacgtccatgtatgtcaaggaattcttgatcagttcaagccaagacggacatcagtggaccctgttttttcaaaatggcaaggtgaaagtttttcaaggcaatcaggacagcttcacccctgttgttaacagccttgatccacccttactcacaagatacttgagaatccatccccagagttgggtgcaccaaatcgctctccggatggaggtgctgggttgtgaggcccaggacctgtactag

SEQ ID NO: 402 - protein, 1475 aa, Synthetic construct
  source 1..1475: note = Truncated FVIII v35
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNATNVSNNSNTSNDSNVSPPVLKRHQRREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 403 - DNA, 4428 bp, Synthetic construct
  source 1..4428: mol_type = other DNA; note = Gene codifying FVIII v36
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcttctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggatctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcttatgtgaaggtggatagctgccctgaggagccccaactgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcaactggaggaccctgagttccaggccagcaacatcatgcacagcatcaatggctatgtgtttgacagcctgcaactgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctctagctgtgacaagaacactggggactactatgaggacagctatgaggacatctctgcctacctgctgagcaagaacaatgccattgagcccaggagcttcagccagaacgccaccaacgtgagcaacaacagcaacaccagcaacgacagcaacgtgagcccccccgtgctgaagaggcaccagaggagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcttgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagccctttagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaatagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagtctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactag

SEQ ID NO: 404 - DNA, 4428 bp, Synthetic construct
  source 1..4428: mol_type = other DNA; note = Gene codifying FVIII v37
atgcagatcgagctgtccacctgcttcttcctgtgcctgctgcggttctgcttttctgccacacggagatactatctgggagccgtggagctgtcttgggattacatgcagagcgacctgggagagctgccagtggacgcaaggtttccacctagagtgcctaagtccttccccttcaacacctctgtggtgtataagaaaaccctgttcgtggagtttacagatcacctgttcaacatcgccaagccaaggccaccatggatgggcctgctgggaccaacaatccaggcagaggtgtacgacaccgtggtcatcaccctgaagaacatggccagccaccccgtgagcctgcacgcagtgggcgtgtcctactggaaggccagcgagggagccgagtatgacgatcagacctctcagcgcgagaaggaggacgataaggtgtttccaggcggcagccacacatacgtgtggcaggtgctgaaggagaatggccccatggccagcgatcctctgtgcctgacctacagctatctgtcccacgtggatctggtgaaggatctgaacagcggcctgatcggggccctgctggtgtgcagagagggctccctggccaaggagaaaacccagacactgcacaagttcatcctgctgttcgccgtgtttgacgagggcaagtcttggcacagcgagacaaagaattctctgatgcaggacagggatgccgcaagcgccagggcatggccaaagatgcacacagtgaacggctacgtgaataggtccctgcccggcctgatcggctgtcacagaaagagcgtgtactggcacgtgatcggcatgggcaccacaccagaggtgcactccatcttcctggagggccacacctttctggtgaggaaccacaggcaggccagcctggagatcagccccatcaccttcctgacagcccagaccctgctgatggatctgggccagttcctgctgttttgccacatcagctcccaccagcacgatggcatggaagcctacgtgaaggtggactcctgccccgaggagccccagctcagaatgaagaacaatgaggaggccgaggactatgacgatgacctgaccgactccgagatggatgtggtgcggttcgatgacgataacagcccatcctttatccagatcagaagcgtggccaagaagcaccccaagacatgggtgcactacatcgccgccgaggaggaggactgggattatgccccactggtgctggcccccgacgatagatcctacaagtctcagtatctgaacaatggcccccagcggatcggcagaaagtacaagaaggtgaggttcatggcctatacagatgagacattcaagacacgcgaggccatccagcacgagagcggcatcctgggacctctgctgtacggagaagtgggcgacaccctgctgatcatctttaagaaccaggccagccggccctacaatatctatccacacggcatcacagatgtgcgccctctgtacagccggcggctgcccaagggagtgaagcacctgaaggacttcccaatcctgcccggcgagatctttaagtataagtggaccgtgacagtggaggatggccccaccaagtccgaccctaggtgcctgacacgctactattctagcttcgtgaatatggagagggatctggcaagcggcctgatcggacctctgctgatctgttacaaggagagcgtggatcagcggggcaaccagatcatgtccgacaagagaaatgtgatcctgttcagcgtgttcgacgagaaccggagctggtatctgaccgagaacatccagagattcctgcctaatccagccggcgtgcagctcgaggatccagagtttcaggccagcaacatcatgcactccatcaatggctacgtgttcgacagcctgcagctcagcgtgtgcctgcacgaggtggcctactggtatatcctgtccatcggagcccagaccgatttcctgagcgtgttcttttccggctacaccttcaagcacaagatggtgtatgaggacaccctgacactgttccctttttccggcgagacagtgttcatgtctatggagaatcccggcctgtggatcctgggctgccacaacagcgacttcagaaacagaggcatgaccgccctgctgaaggtgtcctcttgtgacaagaacacaggcgactactatgaggattcttacgaggacatcagcgcctatctgctgtccaagaacaatgccatcgagcctcgctcttttagccagaatgccaccaacgtgagcaacaacagcaacaccagcaacgacagcaacgtgagccctccagtgctgaagcggcaccagcggcgggagatcaccagaaccacactgcagtctgatcaggaggagatcgactacgacgatacaatcagcgtggagatgaagaaggaggacttcgacatctacgacgaggatgagaaccagagccccaggtccttccagaagaaaacccgccactactttatcgccgccgtggagagactgtgggattatggcatgtcctctagcccccacgtgctgaggaatcgcgcccagtccggctctgtgcctcagttcaagaaggtggtgttccaggagtttaccgacggctcctttacacagcctctgtacaggggagagctgaacgagcacctgggcctgctgggaccatatatcagagccgaggtggaggataacatcatggtgaccttcaggaatcaggccagccggccctatagcttttattcctctctgatcagctacgaggaggaccagaggcagggagccgagccaagaaagaacttcgtgaagcccaatgagacaaagacctacttttggaaggtgcagcaccacatggcccccaccaaggacgagttcgattgcaaagcctgggcctatttttctgacgtggatctggagaaggacgtgcacagcggcctgatcggaccactgctggtgtgccacaccaacacactgaatccagcacacggccggcaggtcaccgtgcaggagttcgccctgttctttaccatctttgatgagacaaagagctggtacttcacagagaacatggagaggaattgccgcgccccttgtaatatccagatggaggacccaaccttcaaggagaactacagatttcacgccatcaatggctatatcatggatacactgcccggcctggtcatggcacaggaccagcggatcagatggtatctgctgtctatgggcagcaacgagaatatccactccatccacttctctggccacgtgtttaccgtgaggaagaaggaggagtataagatggccctgtacaacctgtatcccggcgtgttcgagacagtggagatgctgcctagcaaggcaggaatctggagggtggagtgcctgatcggagagcacctgcacgcaggcatgtctaccctgtttctggtgtacagcaataagtgtcagacacctctgggaatggcaagcggccacatcagggatttccagatcaccgcatccggccagtacggacagtgggccccaaagctggccagactgcactatagcggctccatcaacgcctggagcaccaaggagcctttctcctggatcaaggtggacctgctggccccaatgatcatccacggcatcaagacacagggagcccggcagaagttcagctccctgtacatctcccagtttatcatcatgtattctctggatggcaagaagtggcagacctacagaggcaatagcaccggcacactgatggtgttctttggcaacgtggactctagcggcatcaagcacaacatcttcaatccccctatcatcgcccggtacatcagactgcaccccacccactattccatcaggtctacactgcgcatggagctgatgggctgcgatctgaactcttgtagcatgcctctgggcatggagagcaaggccatctccgacgcccagatcaccgcctcctcttacttcaccaatatgtttgccacatggtccccctctaaggccaggctgcacctgcagggccggtccaacgcatggcggccccaggtgaacaatcccaaggagtggctgcaggtggattttcagaaaaccatgaaggtgacaggcgtgaccacacagggcgtgaagtctctgctgaccagcatgtatgtgaaggagttcctgatcagctcctctcaggacggccaccagtggacactgttctttcagaacggcaaggtgaaggtgttccagggcaatcaggatagctttaccccagtggtgaactccctggacccaccactgctgaccagatacctgcggatccaccctcagagctgggtgcaccagatcgccctgcgcatggaggtgctgggatgcgaggcacaggacctgtattag

SEQ ID NO: 405 - DNA, 4428 bp, Synthetic construct
  source 1..4428: mol_type = other DNA; note = Gene codifying FVIII v38
atgcagatagaactgagcacgtgcttttttttatgtctgctgcgattttgcttcagcgctacccgtcgatattacctgggggctgttgaattgtcttgggattacatgcagtcagatttaggcgaactccctgtggacgcccggttccccccccgcgtcccaaaaagcttcccttttaacacttccgttgtgtataagaagactttatttgtcgagtttacagaccacctctttaatattgccaagcccaggccaccctggatgggactgctcgggcctaccatccaggctgaggtgtatgacacggtggtgatcaccctcaaaaacatggccagccacccggtgtcactccacgctgtcggagtgtcttactggaaggcctccgaaggggccgagtacgatgaccagacctctcagagggagaaagaggacgataaggttttccccggcgggtcccacacttatgtgtggcaggttctgaaggagaacggcccaatggcgtccgatccattgtgtttgacttactcctacttgtctcacgtcgatctggttaaagacttaaactcaggcctgataggggcccttctcgtgtgtagagaaggtagcctcgctaaagagaagactcagaccctgcacaagtttattctgctctttgccgtgtttgatgagggtaagagctggcactccgaaacgaaaaattccctcatgcaggaccgggacgccgcttcagctcgggcctggcctaagatgcacacagtgaacgggtacgtgaatcgcagcctgcctggtttgatcggctgccatcgaaagagcgtgtattggcacgtgatcggaatgggcacaacccctgaggttcatagcatcttcttggagggccatacattcctggttaggaatcataggcaggcttctttggagatcagccccatcacctttctgacagcacaaacactgctgatggacctcggacaatttttgctgttttgtcacatctctagtcaccaacacgacgggatggaagcctatgtcaaagtggactcctgccccgaagaaccccagctccggatgaagaataatgaagaagccgaagactatgatgacgacttgaccgactccgagatggacgtggtgaggtttgacgatgataactctccttcctttatacagatcaggagtgtggctaaaaagcatccgaagacctgggtgcactatatcgccgcagaggaggaagattgggactatgccccactcgtgctggctcctgacgaccgcagttacaaatctcagtacctcaataacggcccccagaggattggtcgaaaatataagaaggtcagatttatggcctatacggacgagacattcaaaacaagggaagccatccagcacgaatccggcatcttgggcccactcctgtacggagaagtcggtgacaccctcctgattatttttaagaaccaggccagccgcccttacaatatatacccccatggcatcaccgatgttaggcctctgtattccaggcgcctgcccaaaggggtaaagcaccttaaagattttcccatcctgcccggcgaaatctttaagtataagtggaccgtgactgttgaagatgggcccactaaatcagatccaaggtgcctgactcggtattacagcagtttcgtcaatatggagagggatctcgcatcaggcctgatcggacctctgctgatctgctataaggagtccgtagatcagaggggaaatcagattatgagcgataagagaaatgtgattctgtttagtgtgtttgatgaaaaccgctcatggtacctgacggagaatatccagcgcttcctcccaaatcccgcaggcgtccagctcgaagaccccgagttccaggccagtaatatcatgcacagcatcaacgggtatgtttttgactccttgcagttgtccgtctgtctgcacgaggtcgcctactggtatattcttagcatcggagctcagacagatttcctcagcgttttctttagtgggtatacatttaagcacaaaatggtatacgaggatactctcaccttgttccctttctcaggcgagactgtctttatgtctatggaaaatcccggcctctggattttagggtgtcataattctgatttccgcaacagaggcatgacagccctgttaaaggtttcctcctgcgataagaatacaggcgattattatgaggattcttacgaggatattagtgcgtatttgctgagtaagaacaacgccatcgagccaaggagcttctcccagaacgctactaatgtgtctaacaactctaacacaagcaacgacagcaatgtttcacccccagtgttgaagagacatcagcgaagagagatcacacggaccacgctgcaatcagatcaggaggagattgattacgacgatactattagcgtggaaatgaagaaagaagatttcgatatatacgacgaggatgaaaaccagtcccctcgcagcttccagaaaaagaccaggcactattttatcgctgccgtggagagactgtgggattacggtatgagtagtagcccacatgtgttgcgaaaccgcgcacagtctggatctgttccccagtttaaaaaggtggtgtttcaagagttcaccgacgggtcttttacccagcctctgtaccgaggtgaactcaacgagcacctcgggctcctgggcccatatattcgagctgaggtagaggataacatcatggtgacatttaggaaccaagcctctcgaccatactccttctacagctcactcatcagctatgaggaggaccaacggcaaggtgccgaaccccggaagaatttcgtgaagcctaatgaaaccaagacatacttctggaaagttcaacatcatatggctcctactaaagacgaatttgattgtaaggcctgggcgtactttagcgacgtcgatctggaaaaggacgtgcatagcgggctcattggtccgcttttggtttgccacaccaacacactgaaccccgctcacgggcgccaggtcaccgtacaggagtttgcactcttcttcaccatattcgatgagactaagtcttggtatttcacagaaaatatggagcgaaactgccgggccccgtgcaacattcagatggaagacccaacgtttaaggagaactaccgctttcacgctattaatgggtacatcatggatacattgccaggcctggtgatggcccaggaccagaggattcgatggtacctgctgagcatgggtagcaatgaaaatattcacagcatccacttctccggtcacgtattcactgtgcgaaagaaagaagagtacaaaatggcgctttacaacctgtacccaggggtgtttgagacagtggagatgctgccaagtaaagccggcatctggagagtcgagtgcttgatcggcgagcatctccacgccggcatgtctactctgtttttagtatattcaaataaatgtcagacccccttagggatggcatctggccacattagggactttcaaattacagcctcaggtcagtacggtcagtgggcccctaaactggctcggcttcactattccggctcaattaatgcgtggtcaacaaaagagccgtttagttggataaaggttgacctgttagcccctatgatcatacatggcattaagacccagggggcacggcaaaagtttagctcactttacatcagtcaatttatcataatgtatagtttggacgggaaaaaatggcagacctaccggggaaattccaccgggacccttatggtattctttggtaatgttgattccagcggcatcaaacacaacatcttcaatcctccaatcatcgcccgctatattcggctgcatcctactcattacagcatccggtctacactgcggatggaactgatgggttgcgatctgaactcatgctccatgcctctcggcatggagtctaaagcaatttccgatgcgcagataactgccagctcatattttaccaacatgttcgctacttggagtccaagcaaggcacgcctgcatttacagggcaggtctaacgcctggcggcctcaggtgaataatccgaaagagtggcttcaggtggattttcagaaaacgatgaaggttactggtgtcacgacccaaggtgtgaagtcccttctgacgtccatgtatgtcaaggaattcttgatcagttcaagccaagacggacatcagtggaccctgttttttcaaaatggcaaggtgaaagtttttcaaggcaatcaggacagcttcacccctgttgttaacagccttgatccacccttactcacaagatacttgagaatccatccccagagttgggtgcaccaaatcgctctccggatggaggtgctgggttgtgaggcccaggacctgtactag

SEQ ID NO: 406 - protein, 1670 aa, Synthetic construct
  source 1..1670: note = Truncated FVIII v39
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLSCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 407 - DNA, 5013 bp, Synthetic construct
  source 1..5013: mol_type = other DNA; note = Gene codifying FVIII v40
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcttctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggatctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgagctgccacatcagcagccaccagcatgatggcatggaggcttatgtgaaggtggatagctgccctgaggagccccaactgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcaactggaggaccctgagttccaggccagcaacatcatgcacagcatcaatggctatgtgtttgacagcctgcaactgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctctagctgtgacaagaacactggggactactatgaggacagctatgaggacatctctgcctacctgctgagcaagaacaatgccattgagcccaggagcttcagccagaacagcaggcaccccagcaccaggcagaagcagttcaacgccaccaccatccccgagaacgacatcgagaagaccgacccctggttcgcccacaggacccccatgcccaagatccagaacgtgagcagcagcgacctgctgatgctgctgaggcagagccccaccccccacggcctgagcctgagcgacctgcaggaggccaagtacgaaaccttcagcgacgaccccagccccggcgccatcgacagcaacaacagcctgagcgagatgacccacttcaggccccaactgcaccacagcggcgacatggtgttcacccccgagagcggcctgcaactgaggctgaacgagaagctgggcaccaccgccgccaccgagctgaagaagctggacttcaaggtgagcagcaccagcaacaacctgatcagcaccatccccagcgacaacctggccgccggcaccgacaacaccagcagcctgggcccccccagcatgcccgtgcactacgacagccaactggacaccaccctgttcggcaagaagtctagccccctgaccgagagcggcggccccctgagcctgagcgaggagaacaacgacagcaagctgctggagagcggcctgatgaacagccaggagagctcttggggcaagaacgtgagcagcagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcttgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagccctttagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaatagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagtctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactag

SEQ ID NO: 408 - DNA, 5013 bp, Synthetic construct
  source 1..5013: mol_type = other DNA; note = Gene codifying FVIII v41
atgcagatcgagctgtccacctgcttcttcctgtgcctgctgcggttctgcttttctgccacacggagatactatctgggagccgtggagctgtcttgggattacatgcagagcgacctgggagagctgccagtggacgcaaggtttccacctagagtgcctaagtccttccccttcaacacctctgtggtgtataagaaaaccctgttcgtggagtttacagatcacctgttcaacatcgccaagccaaggccaccatggatgggcctgctgggaccaacaatccaggcagaggtgtacgacaccgtggtcatcaccctgaagaacatggccagccaccccgtgagcctgcacgcagtgggcgtgtcctactggaaggccagcgagggagccgagtatgacgatcagacctctcagcgcgagaaggaggacgataaggtgtttccaggcggcagccacacatacgtgtggcaggtgctgaaggagaatggccccatggccagcgatcctctgtgcctgacctacagctatctgtcccacgtggatctggtgaaggatctgaacagcggcctgatcggggccctgctggtgtgcagagagggctccctggccaaggagaaaacccagacactgcacaagttcatcctgctgttcgccgtgtttgacgagggcaagtcttggcacagcgagacaaagaattctctgatgcaggacagggatgccgcaagcgccagggcatggccaaagatgcacacagtgaacggctacgtgaataggtccctgcccggcctgatcggctgtcacagaaagagcgtgtactggcacgtgatcggcatgggcaccacaccagaggtgcactccatcttcctggagggccacacctttctggtgaggaaccacaggcaggccagcctggagatcagccccatcaccttcctgacagcccagaccctgctgatggatctgggccagttcctgctgagctgccacatcagctcccaccagcacgatggcatggaagcctacgtgaaggtggactcctgccccgaggagccccagctcagaatgaagaacaatgaggaggccgaggactatgacgatgacctgaccgactccgagatggatgtggtgcggttcgatgacgataacagcccatcctttatccagatcagaagcgtggccaagaagcaccccaagacatgggtgcactacatcgccgccgaggaggaggactgggattatgccccactggtgctggcccccgacgatagatcctacaagtctcagtatctgaacaatggcccccagcggatcggcagaaagtacaagaaggtgaggttcatggcctatacagatgagacattcaagacacgcgaggccatccagcacgagagcggcatcctgggacctctgctgtacggagaagtgggcgacaccctgctgatcatctttaagaaccaggccagccggccctacaatatctatccacacggcatcacagatgtgcgccctctgtacagccggcggctgcccaagggagtgaagcacctgaaggacttcccaatcctgcccggcgagatctttaagtataagtggaccgtgacagtggaggatggccccaccaagtccgaccctaggtgcctgacacgctactattctagcttcgtgaatatggagagggatctggcaagcggcctgatcggacctctgctgatctgttacaaggagagcgtggatcagcggggcaaccagatcatgtccgacaagagaaatgtgatcctgttcagcgtgttcgacgagaaccggagctggtatctgaccgagaacatccagagattcctgcctaatccagccggcgtgcagctcgaggatccagagtttcaggccagcaacatcatgcactccatcaatggctacgtgttcgacagcctgcagctcagcgtgtgcctgcacgaggtggcctactggtatatcctgtccatcggagcccagaccgatttcctgagcgtgttcttttccggctacaccttcaagcacaagatggtgtatgaggacaccctgacactgttccctttttccggcgagacagtgttcatgtctatggagaatcccggcctgtggatcctgggctgccacaacagcgacttcagaaacagaggcatgaccgccctgctgaaggtgtcctcttgtgacaagaacacaggcgactactatgaggattcttacgaggacatcagcgcctatctgctgtccaagaacaatgccatcgagcctcgctcttttagccagaattctcggcacccaagcaccagacagaagcagttcaacgccaccacaatccccgagaatgacatcgagaaaaccgacccttggtttgcccacaggacacccatgcctaagatccagaacgtgagctcctctgatctgctgatgctgctgaggcagtccccaaccccacacggcctgtccctgtctgacctgcaggaggccaagtacgagacattctccgacgatcccagccccggggccatcgacagcaacaatagcctgtccgagatgacccacttcaggccccagctccaccactccggcgacatggtgtttacacctgagagcggcctgcagctccgcctgaatgagaagctgggcaccacagccgccaccgagctgaagaagctggactttaaggtgagctccacctccaacaatctgatcagcacaatcccatccgataacctggccgccggcaccgacaatacatctagcctgggccctccaagcatgcccgtgcactacgattcccagctcgacaccacactgttcggcaagaagtcctctcccctgaccgagagcggcggacctctgtctctgagcgaggagaacaatgactctaagctgctggagagcggcctgatgaactcccaggagagctcctggggcaagaacgtgagcagccgggagatcaccagaaccacactgcagtctgatcaggaggagatcgactacgacgatacaatcagcgtggagatgaagaaggaggacttcgacatctacgacgaggatgagaaccagagccccaggtccttccagaagaaaacccgccactactttatcgccgccgtggagagactgtgggattatggcatgtcctctagcccccacgtgctgaggaatcgcgcccagtccggctctgtgcctcagttcaagaaggtggtgttccaggagtttaccgacggctcctttacacagcctctgtacaggggagagctgaacgagcacctgggcctgctgggaccatatatcagagccgaggtggaggataacatcatggtgaccttcaggaatcaggccagccggccctatagcttttattcctctctgatcagctacgaggaggaccagaggcagggagccgagccaagaaagaacttcgtgaagcccaatgagacaaagacctacttttggaaggtgcagcaccacatggcccccaccaaggacgagttcgattgcaaagcctgggcctatttttctgacgtggatctggagaaggacgtgcacagcggcctgatcggaccactgctggtgtgccacaccaacacactgaatccagcacacggccggcaggtcaccgtgcaggagttcgccctgttctttaccatctttgatgagacaaagagctggtacttcacagagaacatggagaggaattgccgcgccccttgtaatatccagatggaggacccaaccttcaaggagaactacagatttcacgccatcaatggctatatcatggatacactgcccggcctggtcatggcacaggaccagcggatcagatggtatctgctgtctatgggcagcaacgagaatatccactccatccacttctctggccacgtgtttaccgtgaggaagaaggaggagtataagatggccctgtacaacctgtatcccggcgtgttcgagacagtggagatgctgcctagcaaggcaggaatctggagggtggagtgcctgatcggagagcacctgcacgcaggcatgtctaccctgtttctggtgtacagcaataagtgtcagacacctctgggaatggcaagcggccacatcagggatttccagatcaccgcatccggccagtacggacagtgggccccaaagctggccagactgcactatagcggctccatcaacgcctggagcaccaaggagcctttctcctggatcaaggtggacctgctggccccaatgatcatccacggcatcaagacacagggagcccggcagaagttcagctccctgtacatctcccagtttatcatcatgtattctctggatggcaagaagtggcagacctacagaggcaatagcaccggcacactgatggtgttctttggcaacgtggactctagcggcatcaagcacaacatcttcaatccccctatcatcgcccggtacatcagactgcaccccacccactattccatcaggtctacactgcgcatggagctgatgggctgcgatctgaactcttgtagcatgcctctgggcatggagagcaaggccatctccgacgcccagatcaccgcctcctcttacttcaccaatatgtttgccacatggtccccctctaaggccaggctgcacctgcagggccggtccaacgcatggcggccccaggtgaacaatcccaaggagtggctgcaggtggattttcagaaaaccatgaaggtgacaggcgtgaccacacagggcgtgaagtctctgctgaccagcatgtatgtgaaggagttcctgatcagctcctctcaggacggccaccagtggacactgttctttcagaacggcaaggtgaaggtgttccagggcaatcaggatagctttaccccagtggtgaactccctggacccaccactgctgaccagatacctgcggatccaccctcagagctgggtgcaccagatcgccctgcgcatggaggtgctgggatgcgaggcacaggacctgtattag

SEQ ID NO: 409 - DNA, 5013 bp, Synthetic construct
  source 1..5013: mol_type = other DNA; note = Gene codifying FVIII v42
atgcagatagaactgagcacgtgcttttttttatgtctgctgcgattttgcttcagcgctacccgtcgatattacctgggggctgttgaattgtcttgggattacatgcagtcagatttaggcgaactccctgtggacgcccggttccccccccgcgtcccaaaaagcttcccttttaacacttccgttgtgtataagaagactttatttgtcgagtttacagaccacctctttaatattgccaagcccaggccaccctggatgggactgctcgggcctaccatccaggctgaggtgtatgacacggtggtgatcaccctcaaaaacatggccagccacccggtgtcactccacgctgtcggagtgtcttactggaaggcctccgaaggggccgagtacgatgaccagacctctcagagggagaaagaggacgataaggttttccccggcgggtcccacacttatgtgtggcaggttctgaaggagaacggcccaatggcgtccgatccattgtgtttgacttactcctacttgtctcacgtcgatctggttaaagacttaaactcaggcctgataggggcccttctcgtgtgtagagaaggtagcctcgctaaagagaagactcagaccctgcacaagtttattctgctctttgccgtgtttgatgagggtaagagctggcactccgaaacgaaaaattccctcatgcaggaccgggacgccgcttcagctcgggcctggcctaagatgcacacagtgaacgggtacgtgaatcgcagcctgcctggtttgatcggctgccatcgaaagagcgtgtattggcacgtgatcggaatgggcacaacccctgaggttcatagcatcttcttggagggccatacattcctggttaggaatcataggcaggcttctttggagatcagccccatcacctttctgacagcacaaacactgctgatggacctcggacaatttttgctgagctgtcacatctctagtcaccaacacgacgggatggaagcctatgtcaaagtggactcctgccccgaagaaccccagctccggatgaagaataatgaagaagccgaagactatgatgacgacttgaccgactccgagatggacgtggtgaggtttgacgatgataactctccttcctttatacagatcaggagtgtggctaaaaagcatccgaagacctgggtgcactatatcgccgcagaggaggaagattgggactatgccccactcgtgctggctcctgacgaccgcagttacaaatctcagtacctcaataacggcccccagaggattggtcgaaaatataagaaggtcagatttatggcctatacggacgagacattcaaaacaagggaagccatccagcacgaatccggcatcttgggcccactcctgtacggagaagtcggtgacaccctcctgattatttttaagaaccaggccagccgcccttacaatatatacccccatggcatcaccgatgttaggcctctgtattccaggcgcctgcccaaaggggtaaagcaccttaaagattttcccatcctgcccggcgaaatctttaagtataagtggaccgtgactgttgaagatgggcccactaaatcagatccaaggtgcctgactcggtattacagcagtttcgtcaatatggagagggatctcgcatcaggcctgatcggacctctgctgatctgctataaggagtccgtagatcagaggggaaatcagattatgagcgataagagaaatgtgattctgtttagtgtgtttgatgaaaaccgctcatggtacctgacggagaatatccagcgcttcctcccaaatcccgcaggcgtccagctcgaagaccccgagttccaggccagtaatatcatgcacagcatcaacgggtatgtttttgactccttgcagttgtccgtctgtctgcacgaggtcgcctactggtatattcttagcatcggagctcagacagatttcctcagcgttttctttagtgggtatacatttaagcacaaaatggtatacgaggatactctcaccttgttccctttctcaggcgagactgtctttatgtctatggaaaatcccggcctctggattttagggtgtcataattctgatttccgcaacagaggcatgacagccctgttaaaggtttcctcctgcgataagaatacaggcgattattatgaggattcttacgaggatattagtgcgtatttgctgagtaagaacaacgccatcgagccaaggagcttctcccagaactctagacacccctccaccagacagaagcaattcaatgctaccacaatccctgagaacgacatcgaaaagactgacccctggtttgcgcacaggactcccatgcccaagattcaaaatgtgtccagttctgatctgctgatgctgctgaggcagtctccaactccccacgggcttagcctgtctgacctgcaggaagccaagtatgagacatttagcgatgatccctcccctggcgctattgatagtaataacagcctctccgagatgactcacttcaggccccagttacaccattccggcgacatggtcttcacgccagaatccgggcttcaactgcgcctcaacgaaaagctcgggacaaccgccgcaaccgagcttaaaaagctcgacttcaaagtctctagcaccagcaataacctgattagtacaatccctagtgacaatttggccgcgggaacagacaacacctcttccctgggccctccatcaatgcctgtccactacgattcccagctcgataccactctttttggaaaaaaatcttccccactgactgagtccggcggccctctgtccttatctgaggaaaacaacgacagcaagctcctggaaagcgggcttatgaacagtcaagagtcttcttggggtaagaacgtgtctagtagagagatcacacggaccacgctgcaatcagatcaggaggagattgattacgacgatactattagcgtggaaatgaagaaagaagatttcgatatatacgacgaggatgaaaaccagtcccctcgcagcttccagaaaaagaccaggcactattttatcgctgccgtggagagactgtgggattacggtatgagtagtagcccacatgtgttgcgaaaccgcgcacagtctggatctgttccccagtttaaaaaggtggtgtttcaagagttcaccgacgggtcttttacccagcctctgtaccgaggtgaactcaacgagcacctcgggctcctgggcccatatattcgagctgaggtagaggataacatcatggtgacatttaggaaccaagcctctcgaccatactccttctacagctcactcatcagctatgaggaggaccaacggcaaggtgccgaaccccggaagaatttcgtgaagcctaatgaaaccaagacatacttctggaaagttcaacatcatatggctcctactaaagacgaatttgattgtaaggcctgggcgtactttagcgacgtcgatctggaaaaggacgtgcatagcgggctcattggtccgcttttggtttgccacaccaacacactgaaccccgctcacgggcgccaggtcaccgtacaggagtttgcactcttcttcaccatattcgatgagactaagtcttggtatttcacagaaaatatggagcgaaactgccgggccccgtgcaacattcagatggaagacccaacgtttaaggagaactaccgctttcacgctattaatgggtacatcatggatacattgccaggcctggtgatggcccaggaccagaggattcgatggtacctgctgagcatgggtagcaatgaaaatattcacagcatccacttctccggtcacgtattcactgtgcgaaagaaagaagagtacaaaatggcgctttacaacctgtacccaggggtgtttgagacagtggagatgctgccaagtaaagccggcatctggagagtcgagtgcttgatcggcgagcatctccacgccggcatgtctactctgtttttagtatattcaaataaatgtcagacccccttagggatggcatctggccacattagggactttcaaattacagcctcaggtcagtacggtcagtgggcccctaaactggctcggcttcactattccggctcaattaatgcgtggtcaacaaaagagccgtttagttggataaaggttgacctgttagcccctatgatcatacatggcattaagacccagggggcacggcaaaagtttagctcactttacatcagtcaatttatcataatgtatagtttggacgggaaaaaatggcagacctaccggggaaattccaccgggacccttatggtattctttggtaatgttgattccagcggcatcaaacacaacatcttcaatcctccaatcatcgcccgctatattcggctgcatcctactcattacagcatccggtctacactgcggatggaactgatgggttgcgatctgaactcatgctccatgcctctcggcatggagtctaaagcaatttccgatgcgcagataactgccagctcatattttaccaacatgttcgctacttggagtccaagcaaggcacgcctgcatttacagggcaggtctaacgcctggcggcctcaggtgaataatccgaaagagtggcttcaggtggattttcagaaaacgatgaaggttactggtgtcacgacccaaggtgtgaagtcccttctgacgtccatgtatgtcaaggaattcttgatcagttcaagccaagacggacatcagtggaccctgttttttcaaaatggcaaggtgaaagtttttcaaggcaatcaggacagcttcacccctgttgttaacagccttgatccacccttactcacaagatacttgagaatccatccccagagttgggtgcaccaaatcgctctccggatggaggtgctgggttgtgaggcccaggacctgtactag

SEQ ID NO: 410 - DNA, 225 bp, Synthetic construct
  source 1..225: mol_type = other DNA; note = Liver-spec prom-1
gtctgtctgcacatttcgtagagcgagtgttccgatactctaatctccctaggcaaggttcatattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgac

SEQ ID NO: 411 - DNA, 305 bp, Synthetic construct
  source 1..305: mol_type = other DNA; note = Liver-spec prom-2
cgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaa

SEQ ID NO: 412 - DNA, 182 bp, Synthetic construct
  source 1..182: mol_type = other DNA; note = Liver-spec prom-3
gcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaa

SEQ ID NO: 413 - DNA, 240 bp, Synthetic construct
  source 1..240: mol_type = other DNA; note = Liver-spec prom-4
ttgtgcatgggggtgggggtggggttagaggggaacagctccagatggcaaacatacgcaagggatttagtcaaacaactttttggcaaagatggtatgattttgtaatggggtaggaaccaatgaaatgcgaggtaagtatggttaatgatctacagttattggttaaagaagtatattagagcgagtctttctgcacacagatcacctttcctatcaaccccactagcctctggcaaa

SEQ ID NO: 414 - DNA, 307 bp, Synthetic construct
  source 1..307: mol_type = other DNA; note = Liver-spec prom-5
gggggaggctgctggtgaatattaaccaagatcaccccagttaccggaggagcaaacagggactaagttcacacgcgtggtaccgtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctg

SEQ ID NO: 415 - DNA, 251 bp, Synthetic construct
  source 1..251: mol_type = other DNA; note = Liver-spec prom-6
tgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 416 - DNA, 115 bp, Synthetic construct
  source 1..115: mol_type = other DNA; note = Liver-spec enh-1
ctacctgctgatcgcccggcccctgttcaaacatgtcctaatactctgtctctgcaagggtcatcagtagttttccatcttactcaacatcctcccagtgcttcaaagcacctca

SEQ ID NO: 417 - DNA, 117 bp, Synthetic construct; identical to SEQ ID NO: 344
  source 1..117: mol_type = other DNA; note = Synthetic ITR

SEQ ID NO: 418 - DNA, 117 bp, Synthetic construct
  source 1..117: mol_type = other DNA; note = Synthetic ITR
gctcttcttctctgtacactcactcactcactgatcccacccagtcaaagactgggtgtctggatacctttggtatccagggatcagtgagtgagtgagtgtacagagaagaagagc

SEQ ID NO: 419-425 - skipped (7 entries)

(19) United States
(12) Patent Application Publication
(10) Pub. No.: US 2025/0381294 A1
(43) Pub. Date: Dec. 18, 2025

(54) Compositions of DNA Molecules Encoding Factor VIII, Methods of Making Thereof, and Methods of Use Thereof

(71) Applicant: Anjarium Biosciences AG, Schlieren (CH)

(72) Inventors: Joel De Beer, Schlieren (CH); Nicolas Meier, Schlieren (CH); Jorge Omar Yanez-Cuna, Schlieren (CH)

(21) Appl. No.: 18/728,735

(22) PCT Filed: Jan. 13, 2023
(86) PCT No.: PCT/EP2023/050777

§ 371 Date: Jul. 12, 2024

Related U.S. Application Data
(60) Provisional application No. 63/299,500, filed on Jan. 14, 2022.

(51) Int. Cl. A61K 48/00; A61K 38/37; A61K 45/06; A61P 7/04; C12N 15/85; C12N 15/88

(52) U.S. Cl. CPC A61K 48/005; A61K 38/37; A61K 45/06; A61K 48/0083; A61P 7/04; C12N 15/85; C12N 15/88

(57) ABSTRACT

Provided herein are double strand DNA molecules comprising inverted repeats, expression cassette and one or more restriction sites for nicking endonucleases, the methods of use thereof, and the methods of making therefor.

### PRIORITY

This application claims the benefit of priority to U.S. Ser. No. 63/299,500, filed Jan. 14, 2022, which is incorporated herein by reference in its entirety.

### REFERENCE TO SEQUENCE LISTING SUBMITTED ELECTRONICALLY

This application contains a computer readable Sequence Listing which has been submitted in XML file format with this application, the entire content of which is incorporated by reference herein in its entirety. The Sequence Listing XML file submitted with this application is entitled “14497-009-228_SequenceListing.xml”, was created on Jan. 12, 2023, and is 677,006 bytes in size.

### 1. FIELD

Provided herein are DNA molecules encoding Coagulation Factor VIII, the methods of use thereof, and the methods of making thereof. Also provided are methods of treating bleeding disorders.

### 2. BACKGROUND

Gene therapy aims to introduce genes into target cells to treat or prevent disease. By supplying a transcription cassette with an active gene product (sometimes referred to as a transgene), the application of gene therapy can improve clinical outcomes, as the gene product can result in a gain of positive function effect, a loss of negative function effect, or another outcome, such as in patients suffering from cancer, can have an oncolytic effect. Delivery and expression of a corrective gene in the patient's target cells can be carried out via numerous methods, including non-viral delivery (e.g., liposomal) or viral delivery methods that include the use engineered viruses and viral gene delivery vectors. Among the available virus-derived vectors, also known as viral particles, (e.g., recombinant retrovirus, recombinant lentivirus, recombinant adenovirus, and the like), AAV systems are gaining popularity as a versatile vector in gene therapy.

However, there are several major deficiencies in using viral particles as a gene delivery vector. One major drawback is the dependency on viral life cycle and viral proteins to package the transcription cassette into the viral particles. As a result, use of viral vectors has been limited in terms of size of transgenes (e.g., less than 150,000 Da protein coding capacity for AAV) or the requirement for specific viral sequences to be present to ensure efficient replication and packaging (e.g. Rep-Binding Element), which can in turn destabilize the expression cassette. Thus, more than one viral particle may be required to deliver large transgenes (e.g., transgenes encoding proteins larger than 150,000 Da, or transgenes longer than about 4.7 kb). Use of two or more AAV constructs can increase the risk of re-activation of the AAV genome. Furthermore, use of a viral Rep or Nonstructural Protein 1 Binding Element may increase the risk of vector mobilization in the patient.

The second drawback is that viral particles used for gene therapy are often derived from wild-type viruses to which a subset of the population has been exposed during their lifetime. These patients are found to carry neutralizing antibodies which can in turn hinder gene therapy efficacy as further described in Snyder, Richard O., and Philippe Moullier. Adeno-associated virus: methods and protocols. Totowa, NJ: Humana Press, 2011. For the remaining seronegative patients, the capsids of viral vectors are often immunogenic, preventing re-administration of the viral vector therapy to patients should an initial dose not be sufficient or should the therapy wear off.

As such, there is unmet need for non-viral-based gene therapies as an alternative to viral particles, particularly therapies that delivery large transgenes. There is also a need for the DNA vectors to confer greater stability in cell nuclei, allowing prolonged expression compared to circular plasmid DNA. Additionally, there is unmet need for methods to produce these DNA vectors without the co-presences of a plasmid or DNA sequences that encode for the viral replication machinery (e.g., AAV Rep genes), because these viral proteins or the viral DNA sequences encoding for them can contaminate the isolated DNA of a DNA vector.

Furthermore, there remains an important unmet need for recombinant DNA vectors with improved production and/or expression properties. There is also an unmet need for DNA-based vectors that do not elicit an anti-viral (e.g., viral capsid, toll like receptor activation, etc.) immune response allow for repeat administration without loss of efficacy due to, e.g., neutralizing antibodies) or loss of transgene-expressing cells.

Disorders related to impaired or missing function of clotting FVIII (FVIII), including Hemophilia A, cause blood coagulation defects. Due to the increased bleeding risk, with joints being the anatomical site most often involved, patients suffer from damage to joints and depending on where the bleeding occurs, it could be life-threatening. All joints can potentially be involved, but hemarthrosis usually occurs in large synovial joints (e.g. knee, ankles, and elbows), thus progressively leading to a severe and disabling arthropathy. Currently, disease management involves frequent intravenous injections of recombinant FVIII protein, the frequency being high due to its short half-life. The enzyme replacement must begin as soon as possible after birth and be continued for at least 15 years, if not lifelong. Furthermore, most Hemophilia A patients develop long-term pathologies. Despite recent successes with adeno-associated virus (AAV)-based gene replacement for metabolic diseases, current limitations of AAV-mediated gene transfer still represent a challenge for successful gene therapy in Hemophilia A, including the size of the gene (Leebeek and Miesbach, Gene Therapy for Hemophilia: a review on clinical benefit, limitations and remaining issues, Blood, 2021). Furthermore, loss of transgene over time has been observed in liver directed AAV gene therapies, possibly due to the pathological state of the treated hepatocytes.

Despite the great advances in understanding the molecular biology and diagnosis of Hemophilia A, little progress has been made in developing new treatments for the disorder. There remains a large unmet need for durable disease-modifying therapies in Hemophilia A. Classic treatment of Hemophilia A is by replacement therapy targeting restoration of Factor VIII activity. Replacement therapy for treating Hemophilia A involves restoration of Factor VIII activity to 1 to 5% of normal levels to prevent spontaneous bleeding. There are plasma-derived and recombinant Factor VIII products available to treat bleeding episodes on-demand or to prevent bleeding episodes from occurring by treating prophylactically. Based on the half-life of these products, treatment regimens require frequent intravenous administration. Such frequent administration is painful and inconvenient. Furthermore, the need to prevent long term damage to joints and chronic pain remains unaddressed. There are no approved gene therapies for Hemophilia A, and regular AAV based therapies cannot accommodate the large wildtype transgene nor can they be used by 25% to 40% of patients due to pre-existing antibodies. Other viral gene therapy vectors that may accommodate the large transgene pose the challenge that they can only be administered once, and the resulting Factor VIII (FVIII) expression levels might not be high enough to be efficacious or may be supranormal dose levels cannot be titrated.

Accordingly, there is need in the field for a technology that permits expression of a therapeutic FVIII protein in a cell, tissue, or subject in the need of a treatment of Hemophilia A.

### 3. SUMMARY

In one aspect, provided herein is a method for treating a disease associated with reduced activity of coagulation factor VIII in a human patient, the method comprising administering to the patient a biocompatible carrier (hybridosome) or lipid nanoparticle, wherein the hybridosome or the lipid nanoparticle comprises a DNA molecule comprising an expression cassette comprising a transgene encoding human FVIII or a catalytically active fragment thereof.

Provided herein is a method for treating a disease associated with reduced activity of coagulation factor VIII in a human patient, the method comprising administering to the patient a DNA molecule comprising an expression cassette comprising a transgene encoding human coagulation factor VIII or a catalytically active fragment thereof, wherein the DNA molecule is contained within a single delivery vector.

Provided herein is a method for treating a disease associated with reduced activity of FVIII in a human patient, the method comprising the steps of (i) administering a first dose of a DNA molecule comprising an expression cassette comprising a transgene encoding human FVIII or a catalytically active fragment thereof to the patient and (ii) administering a second dose of the DNA molecule to the patient.

In one embodiment, the first dose of the DNA molecule is administered to the patient at least 3 months, at least 4 months, at least 5 months, at least 6 months, at least 7 months, at least 8 months, at least 9 months, at least 10 months, or at least 11 months before the second dose of the DNA molecule.

In one embodiment, the first dose of the DNA molecule is administered to the patient at least 1 year, at least 2 years, at least 3 years, at least 4 years, at least 5 years, at least 10 years, at least 15 years, or at least 20 years before the second dose of the DNA molecule.

In one embodiment, the first dose of the double-stranded DNA molecule and the second dose of the DNA molecule contain the same amount of the DNA molecule.

In one embodiment, the first dose of the DNA molecule and the second dose of the DNA molecule contain different amounts of the DNA molecule.

In one embodiment, the method further comprises administering one or more additional doses of the DNA molecule.

In one embodiment, the DNA molecule is administered once weekly, biweekly, or monthly.

In one embodiment, the DNA molecule is administered to the patient about every 6 months, about every 12 months, about every 18 months, about every 2 years, about every 3 years, about every 5 years, about every 10 years, about every 15 years or about every 20 years.

In one embodiment, the DNA molecule is administered to the patient for the duration of the life of the patient.

In one embodiment, the patient is an adult patient.

In one embodiment, the patient is a pediatric patient.

In one embodiment, the patient is a pediatric patient when the first dose of the DNA molecule is administered.

In one embodiment, the pediatric patient is an infant.

In one embodiment, the pediatric patient is about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, or about 18 years old.

In one embodiment, the disease is Hemophilia A.

In one embodiment, the transgene comprises a sequence that is at least 60%, at least 70%, at least 80% or at least 90% identical to the sequence set forth in SEQ ID NO: 174, 175, 176, 177, 178,179, 180, 181, 379, 380, 381, 383, 384, 385, 387, 388, 389, 391, 392, 393, 395, 396, 397, 399, 400, 401, 403, 404, 405, 407, 408, or 409.

In one embodiment, the method results in an improvement of one or more of the following clinical symptoms of hemophilia A: superfluous annual bleeding rate, hemophilic arthropathy and irreversible joint damage.

In one embodiment, the method results in a reduction in the number of bleeding episodes per year of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100% in the patient.

In one embodiment, the method results in an improvement in blood coagulation cascade function of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100% in a patient as determined by coagulation function tests.

In one embodiment, the method results in a reduction in the number of joint bleeds per year of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100% in the patient.

In one embodiment, the method results in a clinical improvement of about 10%, about 20%, about 30%, about 40%, about 50%, about 60%, about 70%, about 80%, about 90% or greater than about 95% as measured by one or more of the following coagulation markers: prothrombin time test, partial thromboplastin time and clotting factor tests.

In one embodiment, the method results in a clinical improvement of about 10%, about 20%, about 30%, about 40%, about 50%, about 60%, about 70%, about 80%, about 90% or greater than about 95% as measured by the levels of FVIII in the plasma of the patient.

In one embodiment, the method results in FVIII protein activity of about 1-10%, about 10-20%, about 20-30%, about 30-40%, about 40-50%, about 50-60%, about 60-70%, about 70-80%, or about 80-90% of the biological activity level of the native FVIII protein.

In one embodiment, the DNA molecule is detectable in the hepatocytes of the patient by quantitative real-time PCR.

Provided herein is a double-stranded DNA molecule comprising in 5′ to 3′ direction of the top strand:
(a) a first inverted repeat, wherein a first and a second restriction site for nicking endonuclease are arranged on opposite strands in proximity of the first inverted repeat such that nicking results in a top strand 5′ overhang comprising the first inverted repeat upon separation of the top from the bottom strand of the first inverted repeat;
(b) an expression cassette comprising a transgene encoding human FVIII or a catalytically active fragment thereof; and
(c) a second inverted repeat, wherein a third and a fourth restriction site for nicking endonuclease are arranged on opposite strands in proximity of the second inverted repeat such that nicking results in a top strand 3′ overhang comprising the second inverted repeat upon separation of the top from the bottom strand of the second inverted repeat.

Provided herein is a double strand DNA molecule comprising in 5′ to 3′ direction of the top strand:
(a) a first inverted repeat, wherein a first and a second restriction site for nicking endonuclease are arranged on opposite strands in proximity of the first inverted repeat such that nicking results in a bottom strand 3′ overhang comprising the first inverted repeat upon separation of the top from the bottom strand of the first inverted repeat;
(b) an expression cassette comprising a transgene encoding human FVIII or a catalytically active fragment thereof; and
(c) a second inverted repeat, wherein a third and a fourth restriction site for nicking endonuclease are arranged on opposite strands in proximity of the second inverted repeat such that nicking results in a bottom strand 5′ overhang comprising the second inverted repeat upon separation of the top from the bottom strand of the second inverted repeat.

Provided herein is a double-stranded DNA molecule comprising in 5′ to 3′ direction of the top strand:
(a) a first inverted repeat, wherein a first and a second restriction site for nicking endonuclease are arranged on opposite strands in proximity of the first inverted repeat such that nicking results in a top strand 5′ overhang comprising the first inverted repeat upon separation of the top from the bottom strand of the first inverted repeat;
(b) an expression cassette comprising a transgene encoding human FVIII or a catalytically active fragment thereof; and
(c) a second inverted repeat, wherein a third and a fourth restriction site for nicking endonuclease are arranged on opposite strands in proximity of the second inverted repeat such that nicking results in a bottom strand 5′ overhang comprising the second inverted repeat upon separation of the top from the bottom strand of the second inverted repeat.

Provided herein is a double strand DNA molecule comprising in 5′ to 3′ direction of the top strand:
(a) a first inverted repeat, wherein a first and a second restriction site for nicking endonuclease are arranged on opposite strands in proximity of the first inverted repeat such that nicking results in a bottom strand 3′ overhang comprising the first inverted repeat upon separation of the top from the bottom strand of the first inverted repeat;
(b) an expression cassette comprising a transgene encoding human FVIII or a catalytically active fragment thereof; and
(c) a second inverted repeat, wherein a third and a fourth restriction site for nicking endonuclease are arranged on opposite strands in proximity of the second inverted repeat such that nicking results in a top strand 3′ overhang comprising the second inverted repeat upon separation of the top from the bottom strand of the second inverted repeat.

In one embodiment, the DNA molecule provided herein is an isolated DNA molecule.

In one embodiment, the first, second, third, and fourth restriction sites for nicking endonuclease of a DNA molecule provided herein are all restriction sites for the same nicking endonuclease.

In one embodiment, the first and the second inverted repeats of a DNA molecule provided herein are the same.

In one embodiment, the first and/or the second inverted repeat of a DNA molecule provided herein is an ITR of a parvovirus.

In one embodiment, the first and/or the second inverted repeat of a DNA molecule provided herein is a modified ITR of a parvovirus.

In one embodiment, the parvovirus is a Dependoparvovirus, a Bocaparvovirus, an Erythroparvovirus, a Protoparvovirus, or a Tetraparvovirus.

In one embodiment, the nucleotide sequence of the modified ITR of a DNA molecule provided herein is at least 50%, 60%, 70%, 80%, 90%, 95%, 98%, or at least 99% identical to the ITR of the parvovirus.

In one embodiment, the ITR of a DNA molecule provided herein comprises a viral replication-associated protein binding sequence (“RABS”).

In one embodiment, the RABS comprises a Rep binding sequence.

In one embodiment, the RABS comprises an NS1-binding sequence.

In one embodiment, the ITR of a DNA molecule provided herein does not comprise a RABS.

In one embodiment, the transgene comprises a sequence of SEQ ID NO: 174, 175, 176, 177, 178, 179, 180, 181, 379, 380, 381, 383, 384, 385, 387, 388, 389, 391, 392, 393, 395, 396, 397, 399, 400, 401, 403, 404, 405, 407, 408, or 409.

In one embodiment, a DNA molecule provided herein is such that:
(a) the first nick is within 1, 2, 3, 4, 5, 6, 7, 8, 9, 10, 11, 12, 13, 14, 15, 16, 17, 18, 19, or 20 nucleotides from the 5′ nucleotide of the ITR closing base pair of the first inverted repeat;
(b) the second nick is within 1, 2, 3, 4, 5, 6, 7, 8, 9, 10, 11, 12, 13, 14, 15, 16, 17, 18, 19, or 20 nucleotides from the 3′ nucleotide of the ITR closing base pair of the first inverted repeat;
(c) the third nick is within 1, 2, 3, 4, 5, 6, 7, 8, 9, 10, 11, 12, 13, 14, 15, 16, 17, 18, 19, or 20 nucleotides from the 5′ nucleotide of the ITR closing base pair of the second inverted repeat; and/or
(d) the fourth nick is within 1, 2, 3, 4, 5, 6, 7, 8, 9, 10, 11, 12, 13, 14, 15, 16, 17, 18, 19, or 20 nucleotides from the 3′ nucleotide of the ITR closing base pair of the second inverted repeat.

In one embodiment, a DNA molecule provided herein is such that:
(a) the first nick is within 1, 2, 3, 4, 5, 6, 7, 8, 9, 10, 11, 12, 13, 14, 15, 16, 17, 18, 19, or 20 nucleotides from the 3′ nucleotide of the ITR closing base pair of the first inverted repeat;
(b) the second nick is within 1, 2, 3, 4, 5, 6, 7, 8, 9, 10, 11, 12, 13, 14, 15, 16, 17, 18, 19, or 20 nucleotides from the 5′ nucleotide of the ITR closing base pair of the first inverted repeat;
(c) the third nick is within 1, 2, 3, 4, 5, 6, 7, 8, 9, 10, 11, 12, 13, 14, 15, 16, 17, 18, 19, or 20 nucleotides from the 3′ nucleotide of the ITR closing base pair of the second inverted repeat; and/or
(d) the fourth nick is within 1, 2, 3, 4, 5, 6, 7, 8, 9, 10, 11, 12, 13, 14, 15, 16, 17, 18, 19, or 20 nucleotides from the 5′ nucleotide of the ITR closing base pair of the second inverted repeat.

In some embodiment, a DNA molecule provided herein is such that:
(a) the first nick is within 1, 2, 3, 4, 5, 6, 7, 8, 9, 10, 11, 12, 13, 14, 15, 16, 17, 18, 19, or 20 nucleotides from the 5′ nucleotide of the ITR closing base pair of the first inverted repeat;
(b) the second nick is within 1, 2, 3, 4, 5, 6, 7, 8, 9, 10, 11, 12, 13, 14, 15, 16, 17, 18, 19, or 20 nucleotides from the 3′ nucleotide of the ITR closing base pair of the first inverted repeat;
(c) the third nick is within 1, 2, 3, 4, 5, 6, 7, 8, 9, 10, 11, 12, 13, 14, 15, 16, 17, 18, 19, or 20 nucleotides from the 3′ nucleotide of the ITR closing base pair of the second inverted repeat; and/or
(d) the fourth nick is within 1, 2, 3, 4, 5, 6, 7, 8, 9, 10, 11, 12, 13, 14, 15, 16, 17, 18, 19, or 20 nucleotides from the 5′ nucleotide of the ITR closing base pair of the second inverted repeat.

In some embodiment, a DNA molecule provided herein is such that:
(a) the first nick is within 1, 2, 3, 4, 5, 6, 7, 8, 9, 10, 11, 12, 13, 14, 15, 16, 17, 18, 19, or 20 nucleotides from the 3′ nucleotide of the ITR closing base pair of the first inverted repeat;
(b) the second nick is within 1, 2, 3, 4, 5, 6, 7, 8, 9, 10, 11, 12, 13, 14, 15, 16, 17, 18, 19, or 20 nucleotides from the 5′ nucleotide of the ITR closing base pair of the first inverted repeat;
(c) the third nick is within 1, 2, 3, 4, 5, 6, 7, 8, 9, 10, 11, 12, 13, 14, 15, 16, 17, 18, 19, or 20 nucleotides from the 5′ nucleotide of the ITR closing base pair of the second inverted repeat; and/or
(d) the fourth nick is within 1, 2, 3, 4, 5, 6, 7, 8, 9, 10, 11, 12, 13, 14, 15, 16, 17, 18, 19, or 20 nucleotides from the 3′ nucleotide of the ITR closing base pair of the second inverted repeat.

In one embodiment, the nick is inside the inverted repeat.

In one embodiment, the nick is outside the inverted repeat.

In one embodiment, the DNA molecule is a plasmid.

In one embodiment, the DNA molecule is a linear DNA molecule.

In one embodiment, the plasmid further comprises a bacterial origin of replication.

In one embodiment, the plasmid further comprises a restriction enzyme site in the region 5′ to the first inverted repeat and 3′ to the second inverted repeat wherein the restriction enzyme site is not present in any of the first inverted repeat, second inverted repeat, and the region between the first and second inverted repeats.

In one embodiment, the cleavage with the restriction enzyme results in single strand overhangs that do not anneal at detectable levels under conditions that favor annealing of the first and/or second inverted repeat.

In one embodiment, the plasmid further comprises a fifth and a sixth restriction site for nicking endonuclease in the region 5′ to the first inverted repeat and 3′ to the second inverted repeat, wherein the fifth and sixth restriction sites for nicking endonuclease are:
(a) on opposite strands; and
(b) create a break in the double stranded DNA molecule such that the single strand overhangs of the break do not anneal at detectable levels inter- or intramolecularly under conditions that favor annealing of the first and/or second inverted repeat.

In one embodiment, the fifth and the sixth nick are 1, 2, 3, 4, 5, 6, 7, 8, 9, 10, 11, 12, 13, 14, 15, 16, 17, 18, 19, or 20 nucleotides apart.

In one embodiment, the first, second, third, fourth, fifth, and sixth restriction sites for nicking endonuclease are all target sequences for the same nicking endonuclease.

In one embodiment, the nicking endonuclease that recognizes the first, second, third, and/or fourth restriction site for nicking endonuclease is Nt. BsmAI; Nt. BtsCI; N. ALwl; N. BstNBI; N. BspD6I; Nb. Mva1269I; Nb. BsrDI; Nt. BtsI; Nt. Bsal; Nt. Bpu10I; Nt. BsmBI; Nb. BbvCI; Nt. BbvCI; or Nt. BspQI.

In one embodiment, the nicking endonuclease that recognizes the fifth and sixth restriction site for nicking endonuclease is Nt. BsmAI; Nt. BtsCI; N. ALwl; N. BstNBI; N. BspD6I; Nb. Mva1269I; Nb. BsrDI; Nt. BtsI; Nt. Bsal; Nt. Bpu10I; Nt. BsmBI; Nb. BbvCI; Nt. BbvCI; or Nt. BspQI.

In one embodiment, the nicking endonuclease that recognizes the first, second, third, and/or fourth restriction site for nicking endonuclease is a programmable nicking endonuclease.

In one embodiment, the nicking endonuclease that recognizes the fifth and sixth restriction site for nicking endonuclease is a programmable nicking endonuclease.

In one embodiment, the nicking endonuclease is a Cas nuclease.

In one embodiment, the expression cassette further comprises a promoter operatively linked to a transcription unit.

In one embodiment, the transcription unit comprises an open reading frame.

In one embodiment, the expression cassette further comprises a posttranscriptional regulatory element.

In one embodiment, the expression cassette further comprises a polyadenylation and termination signal.

In one embodiment, the size of the expression cassette is at least 4 kb, at least 4.5 kb, at least 5 kb, at least 5.5 kb, at least 6 kb, at least 6.5 kb, at least 7 kb, at least 7.5 kb, at least 8 kb, at least 8.5 kb, at least 9 kb, at least 9.5 kb, or at least 10 kb.

Provided herein is a kit for expressing a human FVIII in vivo, the kit comprising 0.1 to 500 mg of a DNA molecule provided herein and a device for administering the DNA molecule.

In one embodiment, the device is an injection needle.

Provided herein is a composition comprising one or more DNA molecules provided herein, and a pharmaceutically acceptable carrier.

In one embodiment, the carrier comprises a transfection reagent, a nanoparticle, a hybridosomes, a lipid nanoparticle, or a liposome.

In one embodiment, a composition provided herein is used in medical therapy.

In one embodiment, a composition provided herein is used for preparing or manufacturing a medicament for ameliorating, preventing, delaying onset, or treating a disease or disorder associated with reduced activity of FVIII in a subject need thereof.

### 4. BRIEF DESCRIPTION OF THE DRAWINGS

FIG. 1 depicts the structures of various exemplary hairpins and the structural elements of the hairpins.

FIGS. 2A and 2B depict a linear interaction plot showing exemplary strand conformations and intramolecular forces within the overhang as well as intermolecular forces between the strands and FIG. 2C depicts the expected annealed structure of FIG. 2A and FIG. 2B.

FIG. 3 depicts various exemplary arrangements of hairpins and the location of various restriction sites as well as restriction sites for type II nicking endonucleases in the primary stem of a hairpin

FIG. 4 depicts the structures of various exemplary hairpins and the structural elements of human mitochondrial DNA OriL and OriL derived ITRs.

FIG. 5 depicts the structures of hairpins of an exemplary aptamer and aptamer ITR.

FIG. 6 depicts construct 1 and visualization of DNA products from construct 1 after performing method steps as described in Example 1.

FIG. 7 depicts construct 2 and visualization of DNA products from construct 1 after performing method steps as described in Example 1.

FIGS. 8A-8C depict multiple re-/de-nature cycles as described in Example 2.

FIGS. 9A-9B depict isothermal denaturing of construct 1 as described in Example 3.

FIG. 10 depicts expression level of luciferase from various DNA vector amounts. Cells were transfected with different concentrations of DNA vector with either Hybridosomes or lipid nanoparticles. Luciferase activity was determined 48 h after transfection.

FIGS. 11A-11D depict luciferase expression in dividing and non-dividing cells as described in Section 6.5 (Example 5 Expression in dividing and non-dividing cells). FIGS. 11A and 11B depict expression of non-secreted Turboluc (construct 1) in dividing (11A) and non-dividing (11B) cells. For non-secreted Turboluc (construct 1), luciferase activity peaks in dividing cells on day 2, while in non-dividing cells the expression continues to increase.

FIGS. 11C and 11D depict expression of secreted Turboluc (construct 2) in non-dividing (11C) and dividing cells (11D). For secreted Turboluc (construct 2), luciferase activity peaks in dividing cells on day 2, while in non-dividing cells the expression increases and then remains stable over 9 days. As a direct comparison, equimolar amounts of full circular plasmids encoding construct 2 were also transfected and as seen in FIGS. 11C and 11D, generally a lower luciferase activity was recorded, indicating improved nuclear delivery of the purified construct 2 with folded ITRs.

FIG. 12 depicts a sequence alignment of ITRs derived from AAV1 highlighting sequence modifications to generate recognition sites for different nicking endonucleases recognition sites.

FIG. 13 depicts a sequence alignment of ITRs derived from AAV2 highlighting sequence modifications to generate recognition sites for different nicking endonucleases recognition sites.

FIG. 14 depicts a sequence alignment of ITRs derived from AAV3 highlighting sequence modifications to generate recognition sites for different nicking endonucleases recognition sites.

FIG. 15 depicts a sequence alignment of ITRs derived from AAV4 Left highlighting sequence modifications to generate recognition sites for different nicking endonucleases recognition sites.

FIG. 16 depicts a sequence alignment of ITRs derived from AAV4 Right highlighting sequence modifications to generate recognition sites for different nicking endonucleases recognition sites.

FIG. 17 depicts a sequence alignment of ITRs derived from AAV5 highlighting sequence modifications to generate recognition sites for different nicking endonucleases recognition sites.

FIG. 18 depicts a sequence alignment of ITRs derived from AAV7 Left highlighting sequence modifications to generate recognition sites for different nicking endonucleases recognition sites.

FIGS. 19A and 19B depict an agarose gel showing the successful ligation of the DNA construct and the corresponding luciferase expression in non-dividing hepatocytes transfected with hybridosomes encapsulating the ligated construct and non-ligated construct as well as parental plasmid, respectively.

FIG. 20 depicts expression over time of luciferase by non-dividing cells transfected with equimolar amounts of hairpin-ended DNA molecules encoding a secreted luciferase encapsulated in LNPs or Hybridosomes.

FIG. 21 depicts the percentage of RFP positive color switch HEK293 cells following 72 h of transfection of hairpin ended DNA encoding Cre recombinase delivered by lipid nanoparticles, hybridosomes and jetprime as described in Example 9.

FIGS. 22A and 22B depict an agarose gel showing the successful formation of hairpin ended DNA from plasmids comprising right and left ITRs with a wild type AAV RBE compared to mutants in which the RBE was substituted to the corresponding sequences shown in the figure. The luciferase expression in non-dividing hepatocytes transfected with corresponding ITR sequences is shown in FIG. 22B.

FIGS. 23A and 23B: illustrates a further exemplary cloning method (FIG. 24A) and the resulting map of a plasmid (FIG. 23B) from which hairpinned inverted repeat DNA molecules as disclosed herein can be prepared by performing method steps as described in Example 11. In this example, six restriction sites for nicking endonuclease are placed in the region 5′ to the left ITR and 3′ to the right ITR.

FIGS. 24A and 24B depicts and visualizes the products from nicking, denaturing/annealing and exonuclease digestions starting from the plasmid depicted in FIG. 23B on an agarose gel as well as the luminescence readout of said product transfected.

FIGS. 25A and 25B depict and visualizes the products from nicking, denaturing/annealing and exonuclease digestions of constructs encoding FVIII described in Example 12 on an agarose gel. For the DNA construct encoding the truncated FVIII, the agarose gel (FIG. 25A) shows the nicked plasmid in lane 2, the de/renatured DNA products in lane 3, digestion resistant vector in lane 4 and the purified product in lane 5. For the DNA construct encoding the full length FVIII, the agarose gel (FIG. 25B) shows the nicked plasmid in lane 2, the de/renatured DNA products in lane 3, a single band of digestion resistant vector in lane 4 and the purified product in lane 5.

FIG. 26 depicts the level of FVIII activity after transfection of truncated and full length FVIII of Example 13.

FIG. 27 depicts the level of FVIII activity after transfection of CpG free ITR constructs encoding partial B-domain deleted and partial B domain/linker a3 domain deleted truncated FVIII variants in Huh-7 cells described in Example 14.

FIG. 28 shows the results of FVIII expression from transfected hairpin-ended DNA molecules encoding various FVIII variants and codon optimization as well as the effects on supernatant FVIII concentration (IU/ml) as described in Example 17.

FIG. 29 depicts the results of an in vivo study on the activated partial thromboplastin clotting time at day 3 after administration of FVIII encoding hairpin-ended DNA molecules formulated in LNPs as described in Example 18.

### 5. DETAILED DESCRIPTION

Provided herein are methods and compositions for the treatment of a disease or disorder associated with reduced presence or function of Coagulation Factor VIII (FVIII) in a subject. In some embodiments, the disease associated with reduced presence or function of FVIII is Hemophilia A (Hemophilia A). Such compositions include a hairpin-ended DNA molecule, comprising one or more nucleic acids that encode an FVIII therapeutic protein or fragment thereof. In one embodiment, a composition described herein includes a hairpin-ended DNA molecule comprising one nucleic acid that encode an FVIII therapeutic protein or fragment thereof. In one embodiment, a composition described herein includes a hairpin-ended DNA molecule comprising two, three, four, or more nucleic acids that encode an FVIII therapeutic protein or fragment thereof. Also provided herein are hairpin-ended DNA molecules for the expression of the FVIII protein as described herein comprising one or more nucleic acids that encode for the FVIII protein. Also provided herein are methods of manufacturing hairpin-ended DNA molecules described herein. Also provided herein are methods of treating Hemophilia A using the hairpin-ended DNA provided herein and related pharmaceutical compositions. More specifically, provided herein are methods of treating Hemophilia A comprising administering to a subject in need thereof the hairpin-ended DNA described herein.

Provided herein are methods of making hairpin-ended DNA molecules. Also provided herein are methods of using hairpin-ended DNA molecules, including for example, using hairpin-ended DNA molecules for gene therapies. The various methods of making the hairpin-ended DNA molecules are further described in Section 5.2 below. The various methods of using hairpin-ended DNA molecules are described in Section 5.8 below. The hairpin-ended DNA made by these methods are provided in Section 5.5 below and include hairpinned inverted repeats at the two ends and an expression cassette, each of which are further described below. In some embodiments, the hairpin-ended DNA also include one or two nicks, as further provided below in Section 5.5 below. Hairpin, hairpinned inverted repeats, and the hairpinned ends are described in Section 5.5 below; the inverted repeats that form the hairpinned ends are described in Section 5.4.1 below; the nicks, nicking endonuclease, and restriction sites for nicking endonuclease are described in Sections 5.4.2 and 5.5 below; the expression cassette are described in Sections 5.4.3 and 5.5 below; and the functional properties of the hairpin-ended DNA molecules are described in Section 5.6 below. As such, the disclosure provides hairpin-ended DNA molecules, methods of making thereof, methods of using therefor, with any combination or permutation of the components provided herein.

Also provided herein are parent DNA molecules used in the methods to make the hairpin-ended DNA molecules, which parent DNA molecules include two inverted repeats, two or more restriction sites for nicking endonuclease, and an expression cassette, each of which are further described below. The restriction sites for nicking endonuclease are arranged such that, upon nicking by the nicking endonuclease and denaturing, single strand overhangs with inverted repeat sequences form, which then fold to form hairpins upon annealing, each step as described in Section 5.2. The inverted repeats are described in Section 5.4.1 below; the nicks, nicking endonuclease, and restriction sites for nicking endonuclease are described in Section 5.4.2 below; the expression cassette are described in Section 5.4.3 below. As such, the disclosure provides parent DNA molecules used in the methods of making, with any combination or permutation of the components provided herein.

### 5.1 Definitions

As used herein, the term “isolated” when used in reference to a DNA molecule is intended to mean that the referenced DNA molecule is free of at least one component as it is found in its natural, native, or synthetic environment. The term includes a DNA molecule that is removed from some or all other components as it is found in its natural, native, or synthetic environment. Components of a DNA molecule's natural, native, or synthetic environment include anything in natural native, or synthetic environment that are required for, are used in, or otherwise play a role in the replication and maintenance of the DNA molecule in that environment. Components of a DNA molecule's natural, native, or synthetic environment also include, for example, cells, cell debris, cell organelles, proteins, peptides, amino acids, lipids, polysaccharides, nucleic acids other than the referenced DNA molecule, salts, nutrients for cell culture, and/or chemicals used for DNA synthesis. A DNA molecule of the disclosure can be partly, completely, or substantially free from all of these components or any other components of its natural, native, or synthetic environment from which it is isolated, synthetically produced, naturally produced, or recombinantly produced. Specific examples of isolated DNA molecules include partially pure DNA molecules and substantially pure DNA molecules.

As used herein, the term “delivery vehicle” refers to substance that can be used to administer or deliver one or more agents to a cell, a tissue, or a subject, particular a human subject, with or without the agent(s) to be delivered. A delivery vehicle may preferentially deliver agent(s) to a particular subset or a particular type of cells. The selective or preferential delivery achieved by the delivery vehicle can be achieved the properties of the vehicle or by a moiety conjugated to, associated with, or contained in the delivery vehicle, which moiety specifically or preferentially binds to a particular subset of cells. A delivery vehicle can also increase the in vivo half-life of the agent to be delivered, the efficiency of the delivery of the agent comparing to the delivery without using the delivery vehicle, and/or the bioavailability of the agent to be delivered. Non-limiting examples of a delivery vehicle are hybridosomes, liposomes, lipid nanoparticles, polymersomes, mixtures of natural/synthetic lipids, membrane or lipid extracts, exosomes, viral particles, protein or protein complexes, peptides, and/or polysaccharides.

As used herein, the term “subject” refers to a human or any non-human animal (e.g., mouse, rat, rabbit, dog, cat, cattle, swine, sheep, horse, or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder. In an exemplary embodiment, a subject of the present disclosure is a subject with reduced activity (e.g., resulting from reduced concentration, presence, and/or function) of coagulation factor VIII (FVIII). In a further exemplary embodiment, the subject is a human.

As used herein, the term “therapeutic protein” refers to any polypeptide known in the art that when expressed in a subject for the treatment of a disease or a disorder associated to reduced presence or function of FVIII in a subject (e.g. Hemophilia A) brings about significant, measurable change in expression of a Hemophilia A biomarker or a reduction of a given disease associated symptom.

In some embodiments, the therapeutic protein comprises a protein selected from a clotting factor, a functional fragment thereof, or a combination thereof. As used herein, the term “clotting factor,” refers to proteins, or fragments or analogs thereof, naturally occurring or recombinantly produced which prevent or decrease the duration of a bleeding episode in a subject. In other words, it refers to proteins having pro-clotting activity, such as, for example, those responsible for the conversion of fibrinogen into a mesh of insoluble fibrin causing the blood to coagulate or clot. “Clotting factor” as used herein includes an activated clotting factor, its zymogen, or an activable clotting factor. An “activable clotting factor” is a clotting factor in an inactive form (e.g., in its zymogen form) that is capable of being converted to an active form.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 5.2 Hairpin-Ended DNA Molecules and Methods of Making the Hairpin-Ended DNA Molecules

The methods and compositions described herein involve compositions and methods for delivering a FVIII nucleic acid sequence encoding human FVIII (protein to subjects in need thereof for the treatment of Hemophilia A.

In some embodiments, the polynucleotide molecules provided herein express a human FVIII (collectively or individually referred to herein as “FVIII”, “F8” or “Factor VIII”) or a fragment thereof having antihemophilic factor VIII activity.

In some embodiments, the hairpin-ended DNA molecules of this disclosure can be used in methods for ameliorating, preventing, or treating Hemophilia Ain a subject in need thereof.)

The disease or disorder to be treated herein (e.g., Hemophilia A), may be associated with spontaneous hemorrhage and excessive bleeding after trauma. Over time, the repeated bleeding into muscles and joints, which often begins in early childhood, results in hemophilic arthropathy and irreversible joint damage. This damage is progressive and can lead to severely limited mobility of joints, muscle atrophy and chronic pain. As is understood by the skilled artisan, Hemophilia A may be referred to by any number of alternative names in the art, including, but not limited to, FVIII deficiency, bleeder's disease, or classical hemophilia. Accordingly, Hemophilia A may be used interchangeably with any of these alternative names in the specification, the examples, the drawings, and the claims.

In a further aspect, provided herein are methods for making a hairpin-ended DNA molecule for expressing a human coagulation VIII (FVIII) and/or functional fragments thereof. In one aspect, provided herein is a method for preparing a hairpin-ended DNA molecule, wherein the method comprises: a. amplification of the DNA molecule; b. incubating the DNA molecule with one or more nicking endonuclease recognizing the four restriction sites resulting in at least four nicks; c. denaturing and thereby creating a DNA fragment that comprises the expression cassette and is flanked by the two single strand DNA overhangs; d. annealing the single strand DNA overhangs intramolecularly and thereby creating a hairpinned inverted repeat on both ends of the DNA fragment resulting from step c.

### 5.3 Methods of Making the Hairpin-Ended DNA Molecules

In one aspect, provided herein is a method for preparing a hairpin-ended DNA molecule comprising an expression cassette encoding FVIII (or a functional fragment thereof), wherein the method comprises: a. culturing a host cell comprising the DNA molecule as described in Section 5.4 under conditions resulting in amplification of the DNA molecule; b. releasing the DNA molecule from the host cell; c. incubating the DNA molecule with one or more nicking endonuclease recognizing the four restriction sites for the nicking endonuclease resulting in four nicks; d. denaturing and thereby creating a DNA fragment that comprises the expression cassette encoding FVIII or a functional fragment thereof and is flanked by the two single strand DNA overhangs; e. annealing the single strand DNA overhangs intramolecularly and thereby creating a hairpinned inverted repeat on both ends of the DNA fragment resulting from step d.

In another aspect, provided herein is a method for preparing a hairpin-ended DNA comprising an expression cassette encoding FVIII (or a functional fragment thereof), wherein the method comprises: a. culturing a host cell comprising the plasmid of section 5.4.6 under conditions resulting in amplification of the plasmid; b. releasing the plasmid from the host cell; c. incubating the DNA molecule with one or more nicking endonuclease recognizing the four restriction sites resulting in four nicks; d. denaturing and thereby creating a DNA fragment that comprises the expression cassette encoding FVIII or a functional fragment thereof and is flanked by the two single strand DNA overhangs; e. annealing the single strand DNA overhangs intramolecularly and thereby creating a hairpinned inverted repeat on both ends of the DNA fragment resulting from step d; f. incubating the plasmid or the fragments resulting from step d with the restriction enzyme and thereby cleaving the plasmid or a fragment of the plasmid; and g. incubating the fragments of the plasmid with an exonuclease thereby digesting the fragments of the plasmid except the fragment resulting from step e.

In a further aspect, provided herein is a method for preparing a hairpin-ended DNA comprising an expression cassette encoding FVIII (or a functional fragment thereof), wherein the method comprises: a. culturing a host cell comprising the plasmid of Section 5.4 under conditions resulting in amplification of the plasmid; b. releasing the plasmid from the host cell; c. incubating the DNA molecule with one or more nicking endonuclease recognizing the first, second, third, and fourth restriction sites resulting in four nicks; d. denaturing and thereby creating a DNA fragment that comprises the expression cassette encoding FVIII (or a functional fragment thereof) and is flanked by the two single strand DNA overhangs; e. annealing the single strand DNA overhangs intramolecularly and thereby creating a hairpinned inverted repeat on both ends of the DNA fragment resulting from step d; f. incubating the plasmid or the fragments resulting from step d with one or more nicking endonuclease recognizing the fifth and sixth restriction sites resulting in the break in the double stranded DNA molecule; and g. incubating the fragments of the plasmid with an exonuclease thereby digesting the fragments of the plasmid except the fragment resulting from step e.

In one aspect, provided herein is a method for preparing a hairpin-ended DNA molecule comprising an expression cassette encoding FVIII (or a functional fragment thereof), wherein the method comprises: a. culturing a host cell comprising the DNA molecule as described in Section 5.4 under conditions resulting in amplification of the DNA molecule; b. releasing the DNA molecule from the host cell; c. incubating the DNA molecule with one or more programmable nicking enzyme recognizing the four target sites for the guide nucleic acid resulting in four nicks; d. denaturing and thereby creating a DNA fragment that comprises the expression cassette encoding FVIII (or a functional fragment thereof) and is flanked by the two single strand DNA overhangs; e. annealing the single strand DNA overhangs intramolecularly and thereby creating a hairpinned inverted repeat on both ends of the DNA fragment resulting from step d.

In another aspect, provided herein is a method for preparing a hairpin-ended DNA comprising an expression cassette encoding FVIII (or a functional fragment thereof), wherein the method comprises: a. culturing a host cell comprising the plasmid of section 5.4.6 under conditions resulting in amplification of the plasmid; b. releasing the plasmid from the host cell; c. incubating the DNA molecule with one or more programmable nicking enzyme recognizing the four target sites for the guide nucleic acid resulting in four nicks; d. denaturing and thereby creating a DNA fragment that comprises the expression cassette encoding FVIII or a functional fragment thereof and is flanked by the two single strand DNA overhangs; e. annealing the single strand DNA overhangs intramolecularly and thereby creating a hairpinned inverted repeat on both ends of the DNA fragment resulting from step d; f. incubating the plasmid or the fragments resulting from step d with the restriction enzyme and thereby cleaving the plasmid or a fragment of the plasmid; and g. incubating the fragments of the plasmid with an exonuclease thereby digesting the fragments of the plasmid except the fragment resulting from step e.

In a further aspect, provided herein is a method for preparing a hairpin-ended DNA comprising an expression cassette encoding FVIII (or a functional fragment thereof), wherein the method comprises: a. culturing a host cell comprising the plasmid of Section 5.4 under conditions resulting in amplification of the plasmid; b. releasing the plasmid from the host cell; c. incubating the DNA molecule with one or more programmable nicking enzyme recognizing the first, second, third, and fourth target sites for the guide nucleic acids resulting in four nicks; d. denaturing and thereby creating a DNA fragment that comprises the expression cassette encoding FVIII or a functional fragment thereof and is flanked by the two single strand DNA overhangs; e. annealing the single strand DNA overhangs intramolecularly and thereby creating a hairpinned inverted repeat on both ends of the DNA fragment resulting from step d; f. incubating the plasmid or the fragments resulting from step d with programmable nicking enzyme recognizing the fifth and sixth target sites for the guide nucleic acids resulting in the break in the double stranded DNA molecule; and g. incubating the fragments of the plasmid with an exonuclease thereby digesting the fragments of the plasmid except the fragment resulting from step e. In another embodiment, step f of the paragraph can be replaced with step f: incubating the plasmid or the fragments resulting from step d with one or more nicking endonuclease recognizing the two restriction sites resulting in the break in the double stranded DNA molecule.

In certain embodiments, the DNA molecule that comprise an expression cassette encoding FVIII flanked by inverted repeats (as described in Section 5.4) can be provided by culturing host cells comprising the DNA molecules or the plasmids and releasing the DNA molecules or plasmid from the host cell as provided in the steps a and b in the preceding paragraphs. Alternatively, such DNA molecules can be synthesized in a cell-free system or in a combination of cell-free and host cell-based systems. For example, chemical synthesis of DNA fragments and plasmids of various size and sequences is known and widely used in the art; fragments can be chemically synthesized and then ligated by any means known in the art, or recombined in a host cell. In other embodiments, the DNA molecules or plasmids can be provided by in vitro replication. Various methods can be used for in vitro replication, including amplification by polymerase chain reaction. PCR methods for replicating DNA fragments or plasmids of various sizes are well known and widely used in the art, for example, as described in Molecular Cloning: A Laboratory Manual, 4th Edition, by Michael Green and Joseph Sambrook, ISBN 978-1-936113-42-2 (2012), which is incorporated herein in its entirety by reference. In some embodiments, the method of in vitro replication can be isothermal DNA amplification. In some embodiments, step a and b can be replaced by a step of providing DNA molecules by chemical synthesis or PCR. In other embodiments, step a, b, c, and d can be replaced by providing DNA molecules by chemical synthesis.

In one aspect, methods provided herein can be used to prepare hairpin ended DNA molecules comprising an expression cassette encoding FVIII (or a functional fragment thereof, whereby the methods comprise: a. providing a double stranded DNA molecule as described in Section 5.4; b. incubating the DNA molecule with at least one nicking enzyme in conditions resulting in nicking of the double stranded DNA molecule, thereby creating at least two stoichiometric DNA fragments; c. denaturing the DNA fragments; d. annealing the DNA fragments, whereby at least one DNA fragment comprises the expression cassette and single strand DNA overhangs that can be annealed intramolecularly and thereby creating a hairpinned inverted repeat on both ends of said DNA; e. incubating the DNA fragments with at least one exonuclease thereby digesting the stoichiometric DNA fragments of step b, except the hairpin ended fragment comprising the expression cassette resulting from step d. In specific embodiments, step b. of the method in the paragraph creates at least 2, at least 3, at least 4, at least 5, at least 6 or more stoichiometric fragments. In further embodiments, step b. of the method in the paragraph creates at least two stoichiometric DNA fragments, whereby the DNA fragment comprising the expression cassette is stoichiometrically equivalent to the DNA molecules provided in step a. In some embodiments, the digestion resistant hairpin ended fragment comprising the expression cassette resulting from step e in the paragraph can be approximately stoichiometrically equivalent compared to the DNA molecules provided in step a.

In some embodiments, methods provided herein can be used to prepare hairpin ended DNA molecules comprising an expression cassette encoding FVIII (or a functional fragment thereof), whereby the methods comprise: a. providing a double stranded DNA molecule as described in Section 5.4; b. incubating the DNA molecule with at least one nicking enzyme in conditions resulting in nicking of the double stranded DNA molecule, thereby creating at least two stoichiometric DNA fragments; c. denaturing the DNA fragments into single stranded DNA; d. annealing the sense and antisense strand of a DNA fragment comprising the expression cassette, whereby the sense and/or antisense strand comprises single strand DNA overhangs that can be annealed intramolecularly and thereby creating a hairpinned inverted repeat on both ends of the DNA fragment comprising the expression cassette; e. incubating the DNA fragments with at least one exonuclease thereby digesting the stoichiometric DNA fragments of step b, except the hairpin ended fragment comprising the expression cassette resulting from step d.

In some embodiments, methods provided herein can be used to prepare hairpin ended DNA molecules comprising an expression cassette encoding FVIII (or a functional fragment thereof), whereby the methods comprise: a. providing a double stranded DNA molecule as described in Section 5.4; b. incubating the DNA molecule with at least one nicking enzyme in conditions resulting in nicking of the double stranded DNA molecule; c. denaturing the double stranded DNA into thereby creating at least two stoichiometric DNA fragments; d. annealing the DNA fragments, whereby at least one DNA fragment comprises the expression cassette and single strand DNA overhangs that can be annealed intramolecularly and thereby creating a hairpinned inverted repeat on both ends of said DNA fragment.

In a further aspect, the methods provided herein can be used to prepare hairpin ended DNA molecules encoding FVIII (or a functional fragment thereof), wherein the method comprises at least one pot (e.g., a container, vessel, well, tube, plate, or other receptacle) comprising the double stranded DNA molecule as described in Section 5.4 in an aqueous buffer to which sequentially (i) a nicking enzyme, (ii) a denaturing agent (e.g. a base), (iii) an annealing agent (e.g. an acid) and (iv) an exonuclease is added. The ability to perform the method provided herein as a one-pot reaction, may provide at least a further advantage, in that the method to produce hairpin ended DNA molecules can be completed without the need to purify any intermediates, contaminants (e.g. enzymes) or DNA digestion byproducts (i.e. nucleotides, oligos or single stranded DNA fragments) between the method steps (i) to (iv), thereby offering an favorable method in terms of costs and production failure risks (e.g. by minimizing purification losses, requiring less starting material, tighter control of process variables, etc.).

In further embodiments, methods provided herein can be used to produce hairpin ended DNA molecules encoding FVIII (or a functional fragment thereof), wherein the method comprises a. providing one pot (e.g., a container, vessel, well, tube, plate, or other receptacle) comprising a double stranded DNA molecule as described in Section 5.4 and at least one nicking enzyme in conditions resulting in nicking of the double stranded DNA molecule, b. denaturing and annealing the DNA (e.g. by changing the temperature, pH or buffer composition) and c. adding an exonuclease without needing to purify any intermediates (e.g. between step a and c.). In a specific embodiment, the pot in the method of the paragraph; in step a. comprises at least one species of double stranded DNA molecule (e.g., a plasmid or derivative thereof), at least one species of nicking enzyme and an aqueous buffer and in step c. an aqueous buffer comprising at least one species of hairpin ended DNA, at least one species of nicking enzyme, at least one species of exonuclease and DNA digestion products (e.g. dNMPs, dinucleotides and/or short oligos).

In a further embodiment, the methods provided herein can be used to produce hairpin ended DNA molecules encoding FVIII (or a functional fragment thereof), whereby the methods comprises: a. providing a double stranded DNA molecule as described in Section 5.4 and at least one nicking enzyme in at least one pot (e.g., a container, vessel, well, tube, plate, or other receptacle) under conditions resulting in nicking of the double stranded DNA molecule, b. denaturing the DNA molecule and thereby creating a DNA fragment that comprises the expression cassette and is flanked by the two single strand DNA overhangs; c. annealing the single strand DNA overhangs intramolecularly and thereby creating a hairpinned inverted repeat on both ends of the DNA fragment resulting from step b.; d. adding to the pot an exonuclease thereby digesting the DNA fragments of the DNA molecules in step b, except the fragment resulting from step c.

In some embodiments, methods provided herein can be used to prepare hairpin ended DNA molecules comprising an expression cassette encoding FVIII (or a functional fragment thereof), whereby the methods comprises: a. providing a double stranded DNA molecule as described in Section 5.4 and at least one nicking enzyme in at least one pot (e.g., a container, vessel, well, tube, plate, or other receptacle) under conditions resulting in nicking of the double stranded DNA molecule, thereby creating at least two stoichiometric DNA fragments, b. denaturing the DNA fragments; c. annealing the DNA fragments, whereby at least one DNA fragment comprises single strand DNA overhangs that can be annealed intramolecularly and thereby creating a hairpinned inverted repeat on both ends of the DNA fragment comprising the expression cassette; d. adding to the pot at least one exonuclease thereby digesting the DNA fragments of step a, except the hairpin ended DNA fragment comprising the expression cassette resulting from step c. In further embodiments, in step a. of the method in the paragraph creates at least 2, at least 3, at least 4, at least 5, at least 6 or more stoichiometric fragments.

In some embodiments, methods provided herein can be used to prepare hairpin ended DNA molecules comprising an expression cassette encoding FVIII (or a functional fragment thereof), whereby the methods comprises: a. providing a double stranded DNA molecule as described in Section 5.4 in at least one pot (e.g., a container, vessel, well, tube, plate, or other receptacle), b. adding at least one nicking enzyme to the pot in conditions resulting in nicking of the double stranded DNA molecule, thereby creating at least two stoichiometric DNA fragments, c. denaturing the DNA fragments; d. annealing the DNA fragments, whereby at least one DNA fragment comprises single strand DNA overhangs that can be annealed intramolecularly and thereby creating a hairpinned inverted repeat on both ends of the DNA fragment comprising the expression cassette; e. adding at least one exonuclease to the pot thereby digesting the stoichiometric DNA fragments of step b, except the hairpin ended fragment comprising the expression cassette resulting from step d. In specific embodiments, the pot of the method in the paragraph comprises, at step a. one species of DNA molecule and at step b. at least 2, at least 3, at least 4, at least 5, at least 6 or more stoichiometric fragments compared to the DNA molecule in step a, whereby the fragment comprising the expression cassette is stoichiometrically equivalent to the DNA molecules provided in step a. In specific embodiments, the pot at step b. of the method in the paragraph comprises, at least 2, at least 3, at least 4, at least 5, at least 6 or more stoichiometric fragments compared to the DNA molecule in step a, whereby the fragment comprising the expression cassette is stoichiometrically equivalent to the DNA molecules provided in step a. In a non-limiting example, the pot at step b. of the method in the paragraph comprises three stoichiometric DNA fragments; (i) two fragments devoid of the expression cassette and one fragment comprising the expression cassette, whereby the fragment comprising the expression cassette is stoichiometrically equivalent to the DNA molecule provided in step a. In further specific embodiments, the pot at step e. of the method in the paragraph comprises an amount of digestion resistant hairpin ended DNA molecule that is stoichiometrically equivalent compared to the DNA molecules provided in step a. In specific embodiments, the pot at step e. of the method in the paragraph comprises at most an amount of digestion resistant hairpin ended DNA molecule that is stoichiometrically approximately equivalent compared to the DNA molecules provided in step a. In some embodiments, the pot at step e. of the method in the paragraph comprises at most an amount of digestion resistant hairpin ended DNA molecule that is stoichiometrically approximately equivalent compared to the DNA molecules provided in step a, whereby the total mass of DNA molecules is reduced approximately by the ratio of nucleotides present in hairpin ended DNA molecule comprising the expression cassette divided by the nucleotides present in the DNA molecule provided in step a. In specific embodiments, the pot at step e. of the method in the paragraph comprises an amount of digestion resistant hairpin ended DNA molecule that is stoichiometrically approximately equivalent compared to the expression cassettes of the DNA molecules provided in step a.

In some embodiments, the methods provided herein can be used to prepare hairpin ended DNA molecules comprising an expression cassette encoding FVIII (or a functional fragment thereof), whereby the methods comprises: a. culturing a host cell comprising the DNA molecule as described in Section 5.4 under conditions resulting in amplification of the DNA molecule; b. releasing the DNA molecule from the host cell; c. adding the DNA molecule to least one pot (e.g., a container, vessel, well, tube, plate, or other receptacle); d. adding at least one nicking enzyme to the pot in conditions resulting in nicking of the double stranded DNA molecule, thereby creating at least two stoichiometric DNA fragment; e. denaturing the DNA fragments; f. annealing the DNA fragments, whereby at least one DNA fragment comprises the expression cassette and single strand DNA overhangs that can be annealed intramolecularly and thereby creating a hairpinned inverted repeat on both ends of the DNA fragment; g. adding at least one exonuclease to the pot thereby digesting the stoichiometric DNA fragments of step f, except the hairpin ended fragment comprising the expression cassette resulting from step f. In specific embodiments, the pot at step g. of the method in the paragraph comprises an amount of digestion resistant hairpin ended DNA molecule that is stoichiometrically approximately equivalent compared to the DNA molecules provided in step c.

In some embodiments, the methods provided herein can be used to prepare hairpin ended DNA molecules comprising an expression cassette encoding FVIII (or a functional fragment thereof), whereby the methods comprises: a. culturing a host cell comprising the plasmid as described in Section 5.4 under conditions resulting in amplification of the plasmid; b. releasing the plasmid from the host cell; c. adding the plasmid to least one pot (e.g., a container, vessel, well, tube, plate, or other receptacle); d. adding at least one nicking enzyme to the pot in conditions resulting in nicking of the plasmid, thereby creating at least two stoichiometric DNA fragment; e. denaturing the DNA fragments; f. annealing the DNA fragments, whereby at least one DNA fragment comprises the expression cassette and single strand DNA overhangs that can be annealed intramolecularly and thereby creating a hairpinned inverted repeat on both ends of the DNA fragment; g. adding at least one exonuclease to the pot thereby digesting the stoichiometric DNA fragments of step f, except the hairpin ended fragment comprising the expression cassette resulting from step f. In specific embodiments, the pot at step g. of the method in the paragraph comprises an amount of digestion resistant hairpin ended DNA molecule that is stoichiometrically approximately equivalent compared to the plasmid provided in step c.

The order of the method steps is listed in the methods for illustrative purposes. In certain embodiments, the method steps are performed in the order in which they appear as described herein. In some embodiments, the method steps can be performed in an order different from which they appear as described herein. Specifically, in some embodiments, the steps of the methods of making the hairpin-ended DNA molecules can be performed in the order as they appeared or as alphabetically listed as described herein, from a to e, or from a to g. Alternatively, the steps of the methods of making the hairpin-ended DNA molecules can be performed not in the order as they appear as described herein. In one embodiment, the step c (incubating the DNA molecule with one or more nicking endonuclease recognizing the four restriction sites resulting in four nicks) can be performed before step b (releasing the plasmid from the host cell), when the host cells naturally express, are engineered to express, otherwise contain one or more nicking endonuclease. In another embodiment, step f (incubating the plasmid or the fragments resulting from step d with the restriction enzyme or incubating the plasmid or the fragments resulting from step d with one or more nicking endonuclease) can be performed before step d (denaturing and thereby creating a DNA fragment that comprises the expression cassette encoding FVIII (or a functional fragment thereof) and is flanked by the two single strand DNA overhangs), or before step c (incubating the DNA molecule with one or more nicking endonuclease). Additionally, one or more steps can be combined into one step that perform all the actions of the separate step. In certain embodiments, the step a (culturing a host cell) can be combined with step c (incubating the DNA molecule with one or more nicking endonuclease), when the host cells naturally express, are engineered to express, otherwise contain one or more nicking endonuclease. In other embodiments, step f (incubating the plasmid or the fragments resulting from step d with the restriction enzyme or incubating the plasmid or the fragments resulting from step d with one or more nicking endonuclease) can be combined with step c (incubating the DNA molecule with one or more nicking endonuclease) by incubating with the nicking endonuclease or restriction enzyme recited in step f and c together. Therefore, the disclosure provides that the steps can be performed in various combinations and permutations according to the state of the art.

Additional steps can be added to the methods provided herein, before all the method steps, after all the method steps, or in between any of the method steps. In one embodiment, the methods provided herein further include a step h. repairing the nicks with a ligase to form a circular DNA. In another embodiment, the step h of repairing the nicks with a ligase to form a circular DNA is performed after all the other method steps described herein.

As is further described further below in Sections 5.4.1 and 5.5, the hairpins formed at the end of the DNA molecules is determined by properties the overhang between the restriction sites for nicking endonucleases. Therefore, by designing the properties including the sequence and structural properties of the overhang between the restriction sites for nicking endonucleases according to Sections 5.4.1 and 5.5, the methods can be used to produce 1, 2 or more hairpinned ends. In one embodiment, the methods produce hairpin-ended DNA comprising 1 hairpin end. In another embodiment, the methods produce hairpin-ended DNA consisting of 1 hairpin end. In yet another embodiment, the methods produce hairpin-ended DNA comprising two hairpin ends. In a further embodiment, the methods produce hairpin-ended DNA consisting of two hairpin ends.

The methods provided herein can be used to produce DNA molecules comprising artificial sequences, natural DNA sequences, or sequences having both natural DNA sequences and artificial sequences. In one embodiment, the methods produce hairpin-ended DNA molecules comprising artificial sequences. In another embodiment, the methods produce hairpin-ended DNA molecules comprising natural sequences. In yet another embodiment, the methods produce hairpin-ended DNA molecules comprising both natural sequences and artificial sequences. In certain embodiments, the methods produce hairpin-ended DNA molecules comprising viral inverted terminal repeat (ITR). In yet another embodiment, the methods produce a hairpin-ended DNA molecule comprising hairpinned inverted repeats lacking a RABS. In yet another embodiment, the methods produce a hairpin-ended DNA molecule comprising two hairpinned terminal repeats, wherein both hairpinned inverted repeats lack a RABS. In another embodiment, the methods produce a hairpin-ended DNA molecule comprising two hairpinned inverted repeat, wherein both hairpinned inverted repeats lack a TRS. In a further embodiment, the methods produce a hairpin-ended DNA molecule comprising two hairpinned inverted repeats, wherein both hairpinned inverted repeats lack a RABS and a TRS. In another embodiment, the methods produce a hairpin-ended DNA molecule comprising two hairpinned inverted repeats, wherein both hairpinned inverted repeats promoter activity (e.g., P5 promoter activity) and transcriptional activity (e.g. transcription start sites [TSS]). In another embodiment, the methods produce a hairpin-ended DNA molecule comprising two hairpinned inverted repeats, wherein both hairpinned inverted repeats lack a RABS, promoter activity (e.g., P5 promoter activity), transcriptional activity (e.g. transcription start sites [TSS]) and a TRS. In yet another embodiment, the methods produce a hairpin-ended DNA molecule comprising two hairpinned inverted repeats, wherein both hairpinned inverted repeats lack a RABS. In yet another embodiment, the methods produce a hairpin-ended DNA molecule comprising one or two ITRs lacking a RAPS. In a further embodiment, the methods produce hairpin-ended DNA molecules comprising a viral genome. In some embodiments, the viral genome is an engineered viral genome comprising one or more non-viral genes in the expression cassette. In certain embodiments, the viral genome is an engineered viral genome wherein one or more viral genes have been knocked out. In some specific embodiments, the viral genome is an engineered viral genome wherein the replication-associated protein (“RAP,” i.e., Rep or NS1) gene, capsid (Cap) gene, or both RAP and Cap genes are knocked out. In other embodiments, the viral genome is parvovirus genome. In yet other embodiments, the parvovirus is a Dependoparvovirus, a Bocaparvovirus, an Erythroparvovirus, a Protoparvovirus, or a Tetraparvovirus. In one embodiment, the parvovirus is an adeno-associated virus (for example, AAV1, AAV2, AAV4, AAV5, AAV6, AAV7, AAV8, or AAV9).

The steps performed in the various methods provided herein are described in further details below. The embodiments of host cells and culturing of the host cells are described in Section 5.3.1; the embodiments for the step of releasing the DNA molecules from the host cells are described in Section 5.3.2; the embodiments for the step of denaturing the DNA molecules are described in Section 5.3.3; the embodiments for the step of annealing are described in Section 5.3.5; the embodiments for the step of incubating the DNA molecules with nicking endonucleases or restriction enzymes are described in Section 5.3.4; the embodiments for the step of incubating with exonuclease are described in Section 5.3.6; and the embodiments for the step of ligation are described in Section 5.3.7. As such, the disclosure provides methods comprising permutations and combinations of the various embodiments of the steps described herein.

### 5.3.1 Host Cells and Culturing of the Host Cells

The disclosure provides that various host cells can be cultured to amplify the DNA molecules. A host cell for use in the methods provided herein can be a eukaryotic host cell, a prokaryotic host cell, or any transformable organism that is capable of replicating or amplifying recombinant DNA molecules. In some embodiments, the host cell can be a microbial host cell. In further embodiments, the host cell can be a host microbial cell selected from, bacteria, yeast, fungus, or any of a variety of other microorganism cells applicable to replicating or amplifying DNA molecules. A bacterial host cell can be that of any species selected from Escherichia coli, Klebsiella oxytoca, Anaerobiospirillum succiniciproducens, Actinobacillus succinogenes, Mannheimia succiniciproducens, Rhizobium etli, Bacillus subtilis, Corynebacterium glutamicum, Gluconobacter oxydans, Zymomonas mobilis, Lactococcus lactis, Lactobacillus plantarum, Streptomyces coelicolor, Clostridium acetobutylicum, Pseudomonas fluorescens, and Pseudomonas putida. A yeast or fungus host cell can be that of any species selected from Saccharomyces cerevisiae, Schizosaccharomyces pombe, Kluyveromyces lactis, Kluyveromyces marxiamis, Aspergillus terreus, Aspergillus niger, Pichia pastoris, Rhizopus arrhizus, Rhizopus oryzae, and the like. E. coli is a particularly useful host cell since it is a well characterized microbial cell and widely used for molecular cloning. Other particularly useful host cells include yeast such as Saccharomyces cerevisiae. It is understood that any suitable microbial host cells can be used to amplify the DNA molecules as known in the art.

Similarly, a eukaryotic host cell for use in the methods provided herein can be any eukaryotic cell that is capable of replicating or amplifying recombinant DNA molecules, as known and used in the art. In some embodiments, a host cell for use in the methods provided herein can be a mammalian host cells. In further embodiments, a host cell can be a human or non-human mammalian host cell. In other embodiments, a host cell can be an insect host cell. Some widely used non-human mammalian host cells include CHO, mouse myeloma cell lines (e.g., NS0, SP2/0), rat myeloma cell line (e.g. YB2/0), and BHK. Some widely used human host cells include HEK293 and its derivatives, HT-1080, PER.C6, and Huh-7. In certain embodiments, the host cell is selected from the group consisting of HeLa, NIH3T3, Jurkat, HEK293, COS, CHO, Saos, SF9, SF21, High 5, NS0, SP2/0, PC12, YB2/0, BHK, HT-1080, PER.C6, and Huh-7.

A host cell can be cultured as each host cell is known and cultured in the art. The culturing conditions and culture media for different host cells can be different as is known and practiced in the art. For example, bacterial or other microbial host cells can be cultured at 37° C., at an agitation speed of up to 300 rpm, and with or without forced aeration. Some insect host cells can be optimally cultured generally at 25 to 30° C., with no agitation at an agitation speed of up to 150 rpm, and with or without forced aeration. Some mammalian host cells can be optimally cultured at 37° C., with no agitation or at an agitation speed of up to 150 rpm, and with or without forced aeration. Additionally, conditions for culturing the various host cells can be determined by examining the growth curve of the host cells under various conditions, as is known and practiced in the art. Some widely used host cell culturing media and culturing conditions are described in Molecular Cloning: A Laboratory Manual, 4th Edition, by Michael Green and Joseph Sambrook, ISBN 978-1-936113-42-2 (2012), which is incorporated herein in its entirety by reference.

5.3.2 Releasing the DNA Molecules from Host Cells

DNA molecules can be released from the host cells by various ways as known and practiced in the art. For example, the DNA molecules can be released by breaking up the host cells physically, mechanically, enzymatically, chemically, or by a combination of physical, mechanical, enzymatic and chemical actions. In some embodiments, the DNA molecules can be released from the host cells by subjecting the cells to a solution of cell lysis reagents. Cell lysis reagents include detergents, such as triton, SDS, Tween, NP-40, and/or CHAPS. In other embodiments, the DNA molecules can be released from the host cells by subjecting the host cells to difference in osmolarity, for example, subjecting the host cells to a hypotonic solution. In other embodiments, the DNA molecules can be released from the host cells by subjecting the host cells to a solution of high or low pH. In certain embodiments, the DNA molecules can be released from the host cells by subjecting the host cells to enzyme treatment, for example, treatment by lysozyme. In some further embodiments, the DNA molecules can be released from the host cells by subjecting the host cells to any combinations of detergent, osmolarity pressure, high or low pH, and/or enzymes (e.g., lysozyme).

Alternatively, the DNA molecules can be released from the host cells by exerting physical force on the host cells. In one embodiment, the DNA molecules can be released from the host cells by directly applying force to the host cells, e.g., by using the Waring blender and the Polytron. Waring blender uses high-speed rotating blades to break up the cells and the Polytron draws tissue into a long shaft containing rotating blades. In another embodiment, the DNA molecules can be released from the host cells by applying shear stress or shear force to the host cells. Various homogenizers can be used to force the host cells through a narrow space, thereby shearing the cell membranes. In some embodiments, the DNA molecules can be released from the host cells by liquid-based homogenization. In one specific embodiment, the DNA molecules can be released from the host cells by use a Dounce homogenizer. In another specific embodiment, the DNA molecules can be released from the host cells by use a Potter-Elvehjem homogenizer. In yet another specific embodiment, the DNA molecules can be released from the host cells by use a French press. Other physical forces to release the DNA molecules from host cells include manual grinding, e.g., with a mortar and pestle. In manual grinding, host cells are often frozen, e.g., in liquid nitrogen and then crushed using a mortar and pestle, during which process the tensile strength of the cellulose and other polysaccharides of the cell wall breaks up the host cells.

Additionally, the DNA molecules can be released from the host cells by subjecting the cells to freeze and thaw cycles. In some embodiments, a suspension of host cells is frozen and then thawed for a number of such freeze and thaw cycles. In some embodiments, the DNA molecules can be released from the host cells by applying 1, 2, 3, 4, 5, 6, 7, 8, 9, 10, 11, 12, 13, 14, 15, 16, 17, 18, 19, or 20 freeze and thaw cycles to the host cells.

The above described methods for releasing the DNA molecules from the host cells are not mutually exclusive. Therefore, the disclosure provides that the DNA molecules can be released from the host cells by any combinations of DNA releasing methods provide in this Section 5.3.2.

### 5.3.3 Denaturing the DNA Molecules

DNA molecules can be denatured by various ways as known and practiced in the art. The step of denaturing the DNA molecule can separate the DNA molecule from double strand DNA (dsDNA) into single strand DNA (ssDNA). In separating two DNA strands, the temperature can be increased until the DNA unwinds and the hydrogen bonds that hold the two strands together weaken and finally break. The process of breaking double-stranded DNA into single strands is known as DNA denaturation, or DNA denaturing.

In some embodiments, the step of denaturing the DNA molecule can separate the two DNA strands of one or more segments of the dsDNA molecule, while keeping the other segment(s) of the DNA molecule as dsDNA. In some embodiments, the step of denaturing the DNA molecule can separate all DNA strands of one or more segments of the dsDNA molecule into ssDNA strands. In some further embodiments, the step of denaturing the DNA molecules can separate the dsDNA into ssDNA at the segment between the first and second restriction sites for nicking endonuclease on the top and bottom strand of the DNA (e.g. DNA molecules described in Section 5.4), while keeping the other part of the DNA molecule as dsDNA, thereby creating an overhang between the first and second restriction sites. In certain embodiments, the step of denaturing the DNA molecules can separate the dsDNA into ssDNA at the segment between the third and fourth restriction sites for nicking endonuclease on the top and bottom strand of the DNA (e.g. DNA molecules described in Section 5.4), while keeping the other part of the DNA molecule as dsDNA, thereby creating an overhang between the third and fourth restriction sites. In other embodiment, the step of denaturing the DNA molecules can separate the dsDNA into ssDNA at the segments between the first and second restriction sites and between the third and fourth restriction sites for nicking endonuclease on the top and bottom strand of the DNA (e.g. DNA molecules described in Section 5.4), while keeping the other part of the DNA molecule as dsDNA, thereby (1) breaking the DNA molecule into two daughter DNA molecules and (2) creating an overhang between the first and second restriction sites and an overhang between the third and fourth restriction sites. In one embodiment, the overhang between the first and second restriction sites for nicking endonuclease can be a top strand 5′ overhang. In another embodiment, the overhang between the first and second restriction sites for nicking endonuclease can be a bottom strand 3′ overhang. In yet another embodiment, the overhang between the third and fourth restriction sites for nicking endonuclease can be a top strand 3′ overhang. In a further embodiment, the overhang between the third and fourth restriction sites for nicking endonuclease can be a bottom strand 5′ overhang. In some embodiments, step of denaturing the DNA molecule can separate the DNA molecules in any combinations of the embodiments provided herein.

The overhang can vary in length depending on the distance between the restriction sites for nicking endonuclease. In one embodiment, the overhangs can be identical in length and/or sequences. In another embodiment, the overhangs can be different in length and/or sequences. In some embodiments, a top strand 5′ overhang can be at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or at least 100 nucleotides in length. In other embodiments, a top strand 5′ overhang can be about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, or more nucleotides in length. In certain embodiments, a bottom strand 3′ overhang can be at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or at least 100 nucleotides in length. In further embodiments, a bottom strand 3′ overhang can be about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, or more nucleotides in length. In yet other embodiments, a top strand 3′ overhang can be at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or at least 100 nucleotides in length. In other embodiments, a top strand 3′ overhang can be about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, or more nucleotides in length. In some embodiments, a bottom strand 5′ overhang can be at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or at least 100 nucleotides in length. In other embodiments, a bottom strand 5′ overhang can be about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, or more nucleotides in length.

As is known and practiced in the art, the DNA molecules can be denatured by heat, by changing the pH in the environment of the DNA molecules, by increasing the salt concentration, or by any combination of these and other known means. The disclosure provides that the DNA molecules can be denatured in the methods by using a denaturing condition that selectively separates the dsDNA into ssDNA at the segments between the first and second restriction sites and/or between the third and fourth restriction sites on the top and bottom strand of the DNA, while optionally keeping the other part of the DNA molecule as dsDNA. In some embodiment, the denaturing completely separates the dsDNA into ssDNA. Such selective separating of dsDNA to ssDNA can be performed by controlling the denaturing conditions and/or the time the DNA molecules are subjected to the denaturing conditions. In one embodiment, the DNA molecules are denatured at a temperature of at least 70° C., at least 71° C., at least 72° C., at least 73° C., at least 74° C., at least 75° C., at least 76° C., at least 77° C., at least 78° C., at least 79° C., at least 80° C., at least 81° C., at least 82° C., at least 83° C., at least 84° C., at least 85° C., at least 86° C., at least 87° C., at least 88° C., at least 89° C., at least 90° C., at least 91° C., at least 92° C., at least 93° C., at least 94° C., or at least 95° C. In another embodiment, the DNA molecules are denatured at a temperature of about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., or about 95° C. In one specific embodiment, the DNA molecules are denatured at a temperature of about 90° C.

Other than denaturation by heat, sections or all the DNA molecules provided herein can undergo the denaturation process by addition of various chemical agents such as guanidine, formamide, sodium salicylate, dimethyl sulfoxide, propylene glycol, and urea. These chemical denaturing agents lower the melting temperature by competing for hydrogen bond donors and acceptors with pre-existing nitrogenous base pairs and allow for isothermal denaturing. In some embodiments, chemical agents are able to induce denaturation at room temperature. In some specific embodiment, alkaline agents (e.g., NaOH) can be used to denature DNA by changing pH and removing hydrogen-bond contributing protons. In other embodiments, chemically denaturing the DNA molecules provided herein can be a gentler procedure for DNA stability compared to denaturation induced by heat. In other embodiments, chemically denaturing and renaturing the DNA molecules (e.g., changing the pH) provided herein can be a quicker than by heating. In some embodiments, the DNA of the disclosure can be replicated and nicked in bacteria and denatured simultaneously during the release (e.g., alkali lysis step) from bacteria.

In one embodiment, the DNA molecules are denatured at a pH of at least 10, at least 10.1, at least 10.2, at least 10.3, at least 10.4, at least 10.5, at least 10.6, at least 10.7, at least 10.8, at least 10.9, at least 11, at least 11.1, at least 11.2, at least 11.3, at least 11.4, at least 11.5, at least 11.6, at least 11.7, at least 11.8, at least 11.9, at least 12, at least 12.1, at least 12.2, at least 12.3, at least 12.4, at least 12.5, at least 13, at least 13.5, or at least 14. In another embodiment, the DNA molecules are denatured at a pH of about 10, about 10.1, about 10.2, about 10.3, about 10.4, about 10.5, about 10.6, about 10.7, about 10.8, about 10.9, about 11, about 11.1, about 11.2, about 11.3, about 11.4, about 11.5, about 11.6, about 11.7, about 11.8, about 11.9, about 12, about 12.1, about 12.2, about 12.3, about 12.4, about 12.5, about 13, about 13.5, or about 14. In yet another embodiment, the DNA molecules are denatured at a salt concentration of at least 1M, at least 1.5M, at least 2M, at least 2.5M, at least 3M, at least 3.5M, or at least 4M of salt. In a further embodiment, the DNA molecules are denatured at a salt concentration of about 1M, about 1.5M, about 2M, about 2.5M, about 3M, about 3.5M, or about 4M of salt. In certain embodiments, the DNA molecule is subject to the denaturing condition for at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 minutes. In other embodiments, the DNA molecule is subject to the denaturing condition for about 1, about 2, about 3, about 4, about 5, about 6, about 7, about 8, about 9, about 10, about 11, about 12, about 13, about 14, about 15, about 16, about 17, about 18, about 19, or about 20 minutes. In some embodiments, the DNA molecules can be denatured by any combination of denaturing conditions and duration of denaturing as provided herein.

The denaturing conditions can be determined for the method step to selectively denaturing the segments between the first and second restriction sites and between the third and fourth restriction sites on the top and bottom strand of the DNA, while keeping the other part of the DNA molecule as dsDNA. Such selective denaturing conditions can be determined according to the properties of the DNA segments to be selectively denatured. The stability of the DNA double helix correlates with the length of the DNA segments and the percentage of G/C content. The disclosure provides that the selective denaturing conditions can be determined by the sequence of the DNA segments to be selectively denatured or the resulting sequence of the overhang. For example, the temperature for selective denaturing can be approximately determined as Tm=2° C.×number of A-T pair+4° C.×number of G-C pair for a DNA sequence to be selectively denatured. Other more precise calculations of the Tm are also known and used in the art, for example, as described in Freier S M, et a., Proc Natl Acad Sci, 83, 9373-9377 (1986); Breslauer K J, et al., Proc Natl Acad Sci, 83, 3746-3750 (1986); Panjkovich, A. and Melo, F. Bioinformatics 21:711-722 (2005); Panjkovich, A., et al. Nucleic Acids Res 33: W570-W572 (2005), all of which are herein incorporated in their entireties by reference.

The overhang can comprise various DNA sequences. In one embodiment, the overhang comprises an inverted repeat or a fragment thereof (e.g., at least 50%, 60%, 70%, 75%, 80%, 85%, 90%, 95%, or 99% of an inverted repeat). In another embodiment, the overhang comprises a viral inverted repeat or a fragment thereof (e.g., at least 50%, 60%, 70%, 75%, 80%, 85%, 90%, 95%, or 99% of a viral inverted repeat). In yet another embodiment, the overhang comprises or consists of any embodiments of sequences described in Sections 5.4.1, 5.4.2, 5.4.3, and 5.5. In a further embodiment, the overhang comprises or consists of any one of the sequences as described in Sections 5.4.1 and 5.5. In some embodiments, the overhang does not comprise one or more viral replication related sequences (e.g., RABS, RBE or TRS) as described in Section 5.4.5. In some embodiments, the overhang does not comprise one or more transcriptional activity related sequences (e.g., TSSs or CpG motifs) as described in Section 5.4.5.

5.3.4 Incubating the DNA Molecules with One or More Nicking Endonucleases or Restriction Enzymes

The disclosure provides one or more method steps for incubating the DNA molecules with one or more nicking endonucleases or restriction enzymes as described in Section 5.4.2. Without being bound by the theory, a nicking endonuclease recognizes the restriction sites for the nicking endonuclease in the DNA molecule and cuts only on one strand (e.g., hydrolyzes the phosphodiester bond of a single DNA strand) of the dsDNA at a site that is either within or outside the restriction sites for the nicking endonuclease, thereby creating a nick in the dsDNA. A restriction enzyme, on the other hand, recognizes the restriction sites for the restriction enzyme and cuts both strands of the dsDNA, thereby cleaving DNA molecules at or near the specific restriction sites.

In the various embodiments of compositions and methods provided herein, nicking endonucleases can be methylation-dependent, methylation-sensitive, or methylation-insensitive. Various nicking endonucleases known and practiced in the art are provided herein. In some embodiments, the nicking endonucleases for the compositions and methods provided herein can be naturally occurring nicking endonucleases that are not 5-methylcytosine dependent, including Nb.Bsml, Nb.BbvCI, Nb.BsrDI, Nb.Btsl, Nt.BbvCI, Nt. Alwl, Nt. CviPII, Nt. BsmAI, Nt. Alwl and Nt.BstNBI. Nicking endonucleases for the compositions and methods provided herein can also be engineered from Type IIs restriction enzymes (e.g., Alwl, BpulOI, BbvCI, Bsal, BsmBI, BsmAI, Bsml, BspOJ, Mlyl, Mval269l and Sapl, etc.) and methods of making nicking endonucleases can be found in references for example in, U.S. Pat. Nos. 7,081,358; 7,011,966; 7,943,303; 7,820,424, WO201804514, all of which are herein incorporated in their entirety by reference.

Alternatively, a programmable nicking enzyme can be used for the compositions and methods provided herein instead of nicking endonucleases. Such programmable nicking enzyme include, e.g., Cas9 or a functional equivalent thereof (such as Pyrococcus furiosus Argonaute (PfAgo) or Cpf1). Cas9 contains two catalytic domains, RuvC and HNH. Inactivating one of those domains will generate a programmable nicking enzyme that can replace a nicking endonuclease for the methods and compositions provided herein. In Cas9, the RuvC domain can be inactivated by an amino acid substitution at position D10 (e.g., D10A) and the HNH domain can be inactivated by an amino acid substitution at position H840 (e.g., H840A), or at a position corresponding to those amino acids in other Cas9 equivalent proteins. Such programmable nicking enzyme can also be Argonaute or Type II CRISPR/Cas endonucleases that comprise two components: a nicking enzyme (e.g., a D10A Cas9 nicking enzyme or variant or ortholog thereof) that cleaves the target DNA and a guide nucleic acid e.g., a guide DNA or RNA (gDNA or gRNA) that targets or programs the nicking enzyme to a specific site in the target DNA (see, e.g., Hsu, et al., Nature Biotechnology 2013 31:827-832, which is herein incorporated in its entirety by reference). A programmable nicking enzyme can also be made by fusing a site-specific DNA binding domain (targeting domain) such as the DNA binding domain of a DNA binding protein (e.g., a restriction endonuclease, a transcription factor, a zinc-finger, or another domain that binds to DNA at non-random positions) with a nicking endonuclease so that it acts on a specific, non-random site. As is clear from the foregoing, the programmable cleavage by a programmable nicking enzyme results from targeting domain within or fused to the nicking enzyme or from guide molecules (gDNA or gRNA) that direct the nicking enzyme to a specific, non-random site, which site can be programmed by changing the targeting domain or the guide molecule. Such programmable nicking enzymes can be found in references for example, U.S. Pat. No. 7,081,358 and WO2010021692A, which are herein incorporated in their entireties by reference.

Suitable guide nucleic acid (e.g., gDNA or gRNA) sequences and suitable target sites for the guide nucleic acid have been known and widely utilized in the art. The guide nucleic acid (e.g., gDNA or gRNA) is a specific nucleic acid (e.g. gDNA or gRNA) sequence that recognizes the target DNA region of interest and directs the programmable nicking enzyme (e.g. Cas nuclease) there for editing. The guide nucleic acid (e.g., gDNA or gRNA) is often made up of two parts: targeting nucleic acid, a 15-20 nucleotide sequence complementary to the target DNA, and a scaffold nucleic acid, which serves as a binding scaffold for the programmable nicking enzyme (e.g. Cas nuclease). The suitable target sites for the guide nucleic acid must have two components the complementary sequence to the targeting nucleic acid in the programmable nicking enzyme and an adjacent Protospacer Adjacent Motif (PAM). The PAM serves as a binding signal for the programmable nicking enzyme (e.g., Cas nuclease). Various PAMs have been known, characterized, and utilized in the art, for example as discussed in Daniel Gleditzsch et al., RNA Biol. 16 (4): 504-517 (April 2019); Ryan T. Leenay et al., Mol Cell. 62 (1): 137-147 (Apr. 7, 2016), both of which are herein incorporated in their entirety by reference. Exemplary gRNA and gDNA sequences targeting the primary stem sequence of AAV2 ITRs include such listed in Table 1.

TABLE 1
Exemplary Nicking Endonuclease and
Their Corresponding Restriction Sites

| | |
|---|---|
| SEQ ID | AGCGAGCGAGCGCGCAGAGAGGG |
| NO: 352 | |
| AAV2 wt | |
| gRNA for | |
| Nicking | |
| Cas9 | |
| SEQ ID | GCTCGCTCGCTCGGTG |
| NO: 353 | |
| AAV2 wt | |
| gDNA for | |
| PfAgo | |

Various nicking endonucleases known and used in the art can be used in the methods provided herein. An exemplary list of nicking endonuclease provided as embodiments for the nicking endonuclease for use in the methods and the corresponding restriction sites for some of the nicking endonucleases are described in The Restriction Enzyme Database (known in the art as REBASE), which is available at www.rebase.neb.com/cgi-bin/azlist?nick and incorporated herein in its entirety by reference. In one embodiment, the nicking endonuclease that recognizes the first, second, third, and/or fourth restriction site are all for target sequences for the same nicking endonuclease. In another embodiment, the first, second, third, and fourth restriction sites for nicking endonucleases are target sequences for two different nicking endonucleases, including all possible combinations of arranging the four sites for two different nicking endonuclease target sequences (e.g. the first restriction site for the first nicking endonuclease and the rest for the second nicking endonuclease, the first and second restriction sites for the first nicking endonuclease and the rest for the second nicking endonuclease etc.). In yet another embodiment, the first, second, third, and fourth restriction sites for nicking endonucleases are target sequences for three different nicking endonucleases, including all possible combinations of arranging the four sites for three different endonuclease target sequences. In a further embodiment, the first, second, third, and fourth restriction sites for nicking endonucleases are target sequences for four different nicking endonucleases. In some embodiments, the nicking endonuclease can be any one selected from those listed in Table 2.

TABLE 2
Exemplary Nicking Endonuclease and Their
Corresponding Restriction Sites:

| | Corresponding Restriction Sites |
|---|---|
| | for the Nicking Endonuclease and |
| | Position of Nick Relative to the |
| | Restriction Sites |
| | (Note: 1/none means the nick |
| | is 1 nucleotide 3′ from the |
| Nicking | restriction |
| Endonuclease | sites on the top strand). |
| Nt. BsmAI | GTCTC (1/none) |
| Nt. BtsCI | GGATG (2/none) |
| N. ALwl | GGATC (4/none) |
| N. BstNBI | GAGTC (4/none) |
| N. BspD6I | GAGTC (4/none) |
| Nb. Mva1269I | GAATGC (none/−1) |
| Nb. BsrDI | GCAATG (none/0) |
| Nb. BtsI | GCAGTG (none/0) |
| Nt. BtsI | GCAGTG (2/none) |
| Nt. BsaI | GGTCTC (1/none) |
| Nt. Bpu10I | CCTNAGC (−5/none) |
| Nb.Bpu10I | CCTNAGC (none/−2) |
| Nt. BsmBI | CGTCTC (1/none) |
| Nb. BbvCI | CCTCAGC (none/-2) |
| Nt. BbvCI | CCTCAGC (−5/none) |
| Nt. BspQI | GCTCTTC (1/none) |

The conditions for the various nicking endonuclease to cut one strand of the dsDNA are known for the various nicking endonucleases provided herein, including the temperatures, the salt concentration, the pH, the buffering reagent, the presence or absence of certain detergent, and the duration of incubation to achieve the desired percentage of nicked DNA molecules. These conditions are readily available from the websites or catalogs of various vendors of the nicking endonucleases, e.g., New England BioLabs. The disclosure provides that the step of incubating the DNA molecule with one or more nicking endonuclease is performed according to the incubation conditions as known and practiced in the art. In some embodiments, the step of incubating the DNA molecule with one or more nicking endonuclease is according to the incubation conditions optimized by methods known in the art.

Various restriction enzymes known and used in the art can be used in the methods provided herein. An exemplary list of restriction enzymes provided as embodiments for the restriction enzymes for use in the methods and the corresponding restriction sites for the restriction enzymes are described in the catalog of New England Biolabs, which is available at neb.com/products/restriction-endonucleases and incorporated herein in its entirety by reference. The conditions for the various restriction enzymes to cleave the dsDNA are known for the various restriction enzymes provided herein, including the temperatures, the salt concentration, the pH, the buffering reagent, the presence or absence of certain detergent, and the duration of incubation to achieve the desired percentage of nicked DNA molecules. These conditions are readily available from the websites or catalogs of various vendors of the restriction enzymes, e.g., New England BioLabs. The disclosure provides that the step of incubating the DNA molecule with the restriction enzymes is performed according to the incubation conditions as known and practiced in the art.

### 5.3.5 Annealing

The step of annealing in the methods provided herein is performed to selectively anneal the ssDNA overhang intramolecularly and thereby creating a hairpinned inverted repeat on one end of the DNA fragment (e.g. from Sections 5.4 and 5.5) resulted from the step of denaturing as described above (Section 5.3.3). In certain embodiments, the step of annealing in the methods provided herein is performed to selectively anneal the ssDNA overhangs intramolecularly and thereby creating hairpinned inverted repeats on two ends the DNA fragment (e.g. from Sections 5.4 and 5.5) resulted from the step of denaturing as described above (Section 5.3.3). Without being bound or otherwise limited by the theory, such selective intramolecular annealing of the ssDNA overhangs is achieved because the intramolecular complementary sequences within the ssDNA overhangs make the intramolecular annealing of the ssDNA overhangs thermodynamically and/or kinetically favored over the intermolecular annealing of the ssDNA overhangs.

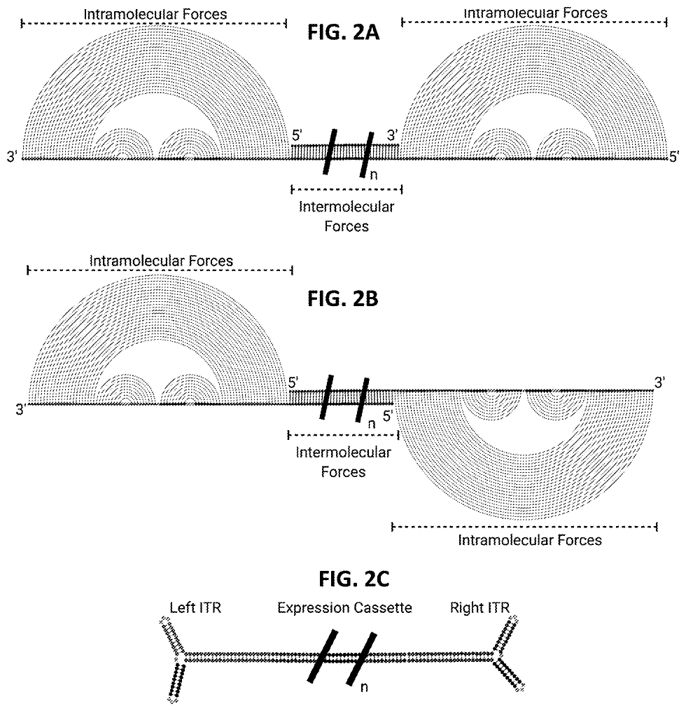

Without being bound or otherwise limited by the theory, it is recognized that certain lengths and/or the sequences of the overhang can make the intramolecular annealing of the ssDNA overhangs thermodynamically and/or kinetically favored over the intermolecular annealing of the ssDNA overhangs. For example, a linear interaction plot showing the intramolecular forces within the overhang and intermolecular forces between the strands as well as the resulting structure is depicted in FIG. 2A-C. The thermodynamics and the kinetics of the annealing of the ssDNA overhang is determined by the enthalpy (ΔH) and the entropy (ΔS), among other factors. The inventors recognize that, as the loss of movement freedom from a free ssDNA overhang to an intramolecularly annealed overhang is less than the loss of movement freedom from free ssDNA overhang to intermolecularly annealed overhang, the entropy loss in an intramolecular annealing is less than the entropy loss in an intramolecular annealing. On the other hand, as the number of complementary nucleotide pairs in an intramolecularly annealed overhang is less than number of complementary nucleotide pairs in an intermolecularly annealed overhang (hence less Watson-Crick and Hoogsteen-type hydrogen bonding), the enthalpy gain in an intramolecular annealing may be less than the enthalpy gain in an intramolecular annealing. The disclosure provides that the ssDNA overhang can be designed to have certain lengths, numbers of complementary nucleotide pairs, and percentage of G-C and A-T pairs, such that the free energy gain (ΔG=ΔH-TΔS) of intramolecular annealing of the overhang is bigger over that of intermolecular annealing, thereby making the intramolecular annealing thermodynamically favored over the intermolecular annealing. The inventors further recognize that, as the nucleotides within the ssDNA overhang have a higher probability of contacting each other than contacting the nucleotides of another ssDNA overhang in molecular motion, the kinetics of intramolecular annealing of the ssDNA overhang can be higher than that of intermolecular annealing. The disclosure provides that even if the intramolecular annealing is thermodynamically disfavored over the intermolecular annealing, the superior kinetics of intramolecular annealing of the ssDNA overhang can result in the formation of intramolecularly annealed overhang over intermolecularly annealed overhang.

The annealing step can be performed at various temperatures to favor the intramolecular annealing over intermolecular annealing. In one embodiment, the ssDNA overhang is annealed at a temperature of at least 15° C., at least 16° C., at least 17° C., at least 18° C., at least 19° C., at least 20° C., at least 21° C., at least 22° C., at least 23° C., at least 24° C., at least 25° C., at least 26° C., at least 27° C., at least 28° C., at least 29° C., at least 30° C., at least 31° C., at least 32° C., at least 33° C., at least 34° C., at least 35° C., at least 36° C., at least 37° C., at least 38° C., at least 39° C., at least 40° C., at least 41° C., at least 42° C., at least 43° C., at least 44° C., at least 45° C., at least 46° C., at least 47° C., at least 48° C., at least 49° C., at least 50° C., at least 51° C., at least 52° C., at least 53° C., at least 54° C., at least 55° C., at least 56° C., at least 57° C., at least 58° C., at least 59° C., or at least 60° C. In another embodiment, the ssDNA overhang is annealed at a temperature of about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° C., about 56° C., about 57° C., about 58° C., about 59° C., or about 60° C. In one specific embodiment, the ssDNA overhang is annealed at a temperature of at least 25° C. In another specific embodiment, the ssDNA overhang is annealed at a temperature of about 25° C. In yet another specific embodiment, the ssDNA overhang is annealed at room temperature.

Additionally, the annealing step can be performed for various durations of time to favor the intramolecular annealing over intermolecular annealing. In certain embodiments, the ssDNA overhang is annealed for at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, or at least 40 minutes. In other embodiments, the ssDNA overhang is annealed for about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, or about 40 minutes. In one specific embodiment, the ssDNA overhang is annealed for at least 20 minutes. In another specific embodiment, the ssDNA overhang is annealed for about 20 minutes.

In some embodiments, annealing can be accomplished by lowering the temperature below the calculated melting temperatures of the sense and antisense sequence pairs. The melting temperature is dependent upon the specific nucleotide base content and the characteristics of the solution being used, e.g., the salt concentration. Melting temperatures for any given sequence and solution combination are readily calculated as known and practiced in the art.

In some embodiments, annealing can be accomplished isothermally by reducing the amount of denaturing chemical agents to allow an interaction between the sense and antisense sequence pairs. The minimum concentration of denaturing chemical agents required to denature the DNA sequence can dependent upon the specific nucleotide base content and the characteristics of the solution being used, e.g., temperature or the salt concentration. The concentration of chemical denaturing agents that do not lead to denaturing for any given sequence and solution combination are readily identified as known and practiced in the art. The concentration of chemical denaturing agents can also be readily modified as known and practiced in the art. For example, the amount of urea can be lowered by dialysis or tangential flow filtration, or the pH can be changed by the addition of acids or bases.

The annealing temperature and the annealing duration for intramolecular annealing correlate with the lengths of the ssDNA overhang, the number of complementary nucleotide pairs, and percentage of G-C and A-T pairs, and the sequence of the ssDNA overhang (the arrangement of the complementary nucleotide pairs). In certain embodiments, an ssDNA overhang provided for the methods provided herein comprises any number of nucleotides in length as described in Section 5.3.3. In certain embodiments, a ssDNA overhang provided for the methods provided herein comprises at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 intramolecularly complementary nucleotide pairs. In some embodiments, a ssDNA overhang provided for the methods provided herein comprises about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50 intramolecularly complementary nucleotide pairs. In some embodiments, a ssDNA overhang provided for the methods provided herein comprises at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, or at least 90% G-C pairs among intramolecularly complementary nucleotide pairs. In certain embodiments, a ssDNA overhang provided for the methods provided herein comprises about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90% G-C pairs among intramolecularly complementary nucleotide pairs.

Additionally, the inventors recognize that the concentration of the DNA molecules, which correlates with the concentration of the overhangs, can affect the equilibrium and kinetics of the intramolecular annealing and the intermolecular annealing of the overhangs. Without being bound or otherwise limited by the theory, when the concentration of the overhang is too high, the probability of the intermolecular contact among the overhangs increases and the kinetic advantage of the intramolecular contact over intermolecular contact seen at lower concentration as discussed above is then diminished.

As discussed above, in some embodiments, intramolecular interactions can occur at a faster rate while intermolecular interactions occur at a slower rate. In some embodiments, base pair interactions involving three or more molecules (e.g., three different strands) occur at the slowest rate. In some embodiments, the kinetic rate of intramolecular interactions versus intermolecular interactions is governed by the concentration of each molecule. In some embodiments, the intramolecular interactions are kinetically faster, or intramolecular forces are larger when the concentration of DNA strands is lower.

Viewed individually, the absolute free energy of forming each complementary domain of IRs or ITRs, may be different, leading to regions of the IR or ITR that may locally fold earlier as the strand transitions from a denatured to annealed state. The presence of locally folded domains (e.g. a central hairpin or branched hairpin like in AAV2 ITRs as described in elsewhere in this Section (Section 5.4.1) and Section 5.5) can reduce the amount of bases available for pairing with other strands and thus can reduce the likelihood of intermolecular annealing or hybridization and shift the equilibrium from intermolecular annealing to intramolecular annealing or ITR formation.

Accordingly, the disclosure provides that the annealing step can be performed at various concentrations to favor the intramolecular annealing over intermolecular annealing. In some embodiments, the ssDNA overhang is annealed at a concentration of no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19, no more than 20, no more than 21, no more than 22, no more than 23, no more than 24, no more than 25, no more than 26, no more than 27, no more than 28, no more than 29, no more than 30, no more than 31, no more than 32, no more than 33, no more than 34, no more than 35, no more than 36, no more than 37, no more than 38, no more than 39, no more than 40, no more than 41, no more than 42, no more than 43, no more than 44, no more than 45, no more than 46, no more than 47, no more than 48, no more than 49, no more than 50, no more than 55, no more than 60, no more than 65, no more than 70, no more than 75, no more than 80, no more than 85, no more than 90, no more than 95, no more than 100, no more than 110, no more than 120, no more than 130, no more than 140, no more than 150, no more than 160, no more than 170, no more than 180, no more than 190, no more than 200, no more than 210, no more than 220, no more than 230, no more than 240, no more than 250, no more than 260, no more than 270, no more than 280, no more than 290, no more than 300, no more than 325, no more than 350, no more than 375, no more than 400, no more than 425, no more than 450, no more than 475, no more than 500, no more than 550, no more than 600, no more than 650, no more than 700, no more than 750, no more than 800, no more than 850, no more than 900, no more than 950, no more than 1000 ng/μl for the DNA molecules. In certain embodiments, the ssDNA overhang is annealed at a concentration of about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, about 300, about 325, about 350, about 375, about 400, about 425, about 450, about 475, about 500, about 550, about 600, about 650, about 700, about 750, about 800, about 850, about 900, about 950, about 1000 ng/μl for the DNA molecules.

Similarly, the disclosure provides that the annealing step can be performed at various molar concentrations to favor the intramolecular annealing over intermolecular annealing. In some embodiments, the ssDNA overhang is annealed at a concentration of no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19, no more than 20, no more than 21, no more than 22, no more than 23, no more than 24, no more than 25, no more than 26, no more than 27, no more than 28, no more than 29, no more than 30, no more than 31, no more than 32, no more than 33, no more than 34, no more than 35, no more than 36, no more than 37, no more than 38, no more than 39, no more than 40, no more than 41, no more than 42, no more than 43, no more than 44, no more than 45, no more than 46, no more than 47, no more than 48, no more than 49, no more than 50, no more than 55, no more than 60, no more than 65, no more than 70, no more than 75, no more than 80, no more than 85, no more than 90, no more than 95, no more than 100, no more than 110, no more than 120, no more than 130, no more than 140, no more than 150, no more than 160, no more than 170, no more than 180, no more than 190, no more than 200, no more than 210, no more than 220, no more than 230, no more than 240, no more than 250, no more than 260, no more than 270, no more than 280, no more than 290, no more than 300, no more than 325, no more than 350, no more than 375, no more than 400, no more than 425, no more than 450, no more than 475, no more than 500, no more than 550, no more than 600, no more than 650, no more than 700, no more than 750, no more than 800, no more than 850, no more than 900, no more than 950, no more than 1000 nM for the DNA molecules. In certain embodiments, the ssDNA overhang is annealed at a concentration of about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, about 300, about 325, about 350, about 375, about 400, about 425, about 450, about 475, about 500, about 550, about 600, about 650, about 700, about 750, about 800, about 850, about 900, about 950, about 1000 nM for the DNA molecules. In some further embodiments, the ssDNA overhang is annealed at a concentration of no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19, no more than 20 μM. In yet other embodiments, the ssDNA overhang is annealed at a concentration of about 1, about 2, about 3, about 4, about 5, about 6, about 7, about 8, about 9, about 10, about 11, about 12, about 13, about 14, about 15, about 16, about 17, about 18, about 19, about 20 μM. In one specific embodiment, the ssDNA overhang is annealed at a concentration of about 10 nM for the DNA molecules. In another specific embodiment, the ssDNA overhang is annealed at a concentration of about 20 nM for the DNA molecules. In yet another specific embodiment, the ssDNA overhang is annealed at a concentration of about 30 nM for the DNA molecules. In a further specific embodiment, the ssDNA overhang is annealed at a concentration of about 40 nM for the DNA molecules. In still another specific embodiment, the ssDNA overhang is annealed at a concentration of about 50 nM for the DNA molecules. In another specific embodiment, the ssDNA overhang is annealed at a concentration of about 60 nM for the DNA molecules. In one specific embodiment, the ssDNA overhang is annealed at a concentration of about 10 ng/μl for the DNA molecules. In another specific embodiment, the ssDNA overhang is annealed at a concentration of about 20 ng/μl for the DNA molecules. In yet another specific embodiment, the ssDNA overhang is annealed at a concentration of about 30 ng/μl for the DNA molecules. In a further specific embodiment, the ssDNA overhang is annealed at a concentration of about 40 ng/μl for the DNA molecules. In one specific embodiment, the ssDNA overhang is annealed at a concentration of about 50 ng/μl for the DNA molecules. In another specific embodiment, the ssDNA overhang is annealed at a concentration of about 60 ng/μl for the DNA molecules. In yet another specific embodiment, the ssDNA overhang is annealed at a concentration of about 70 ng/μl for the DNA molecules. In one specific embodiment, the ssDNA overhang is annealed at a concentration of about 80 ng/μl for the DNA molecules. In another specific embodiment, the ssDNA overhang is annealed at a concentration of about 90 ng/μl for the DNA molecules. In yet another specific embodiment, the ssDNA overhang is annealed at a concentration of about 100 ng/μl for the DNA molecules.

In some embodiments, an ssDNA overhang provided for the methods provided herein comprises any sequences listed in Table 3.

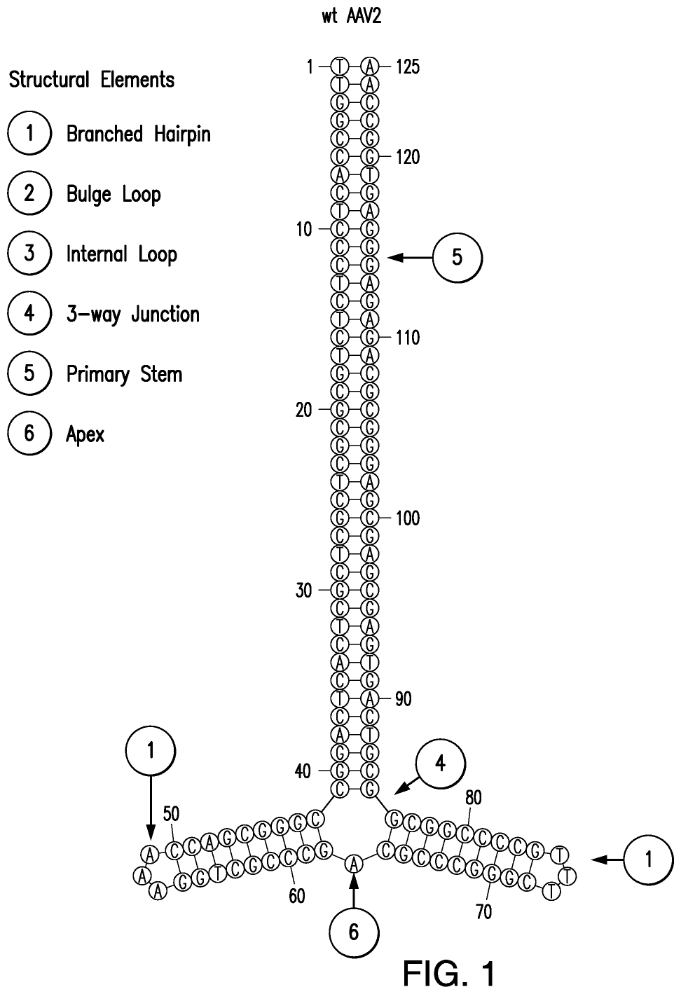

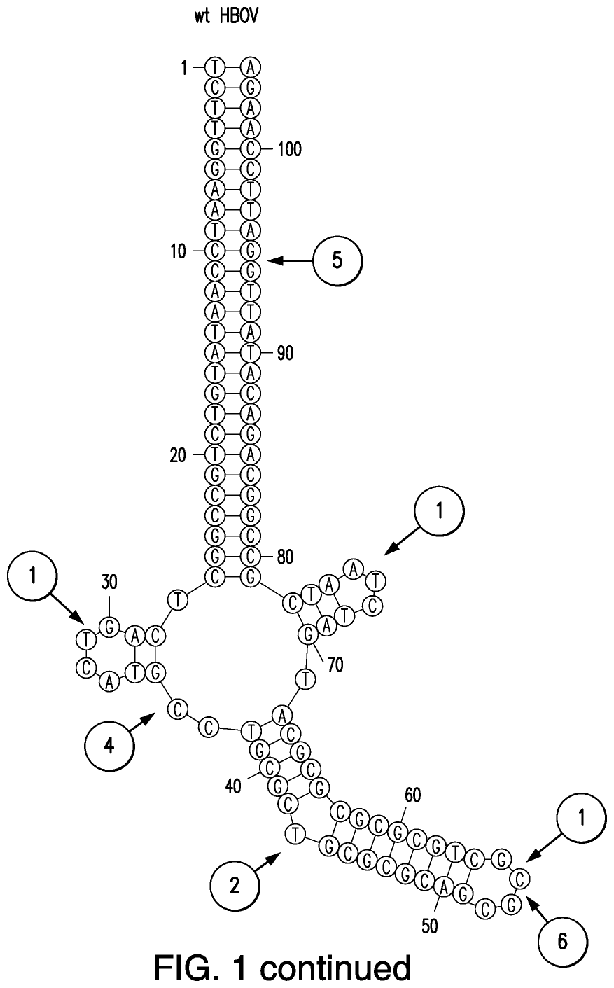

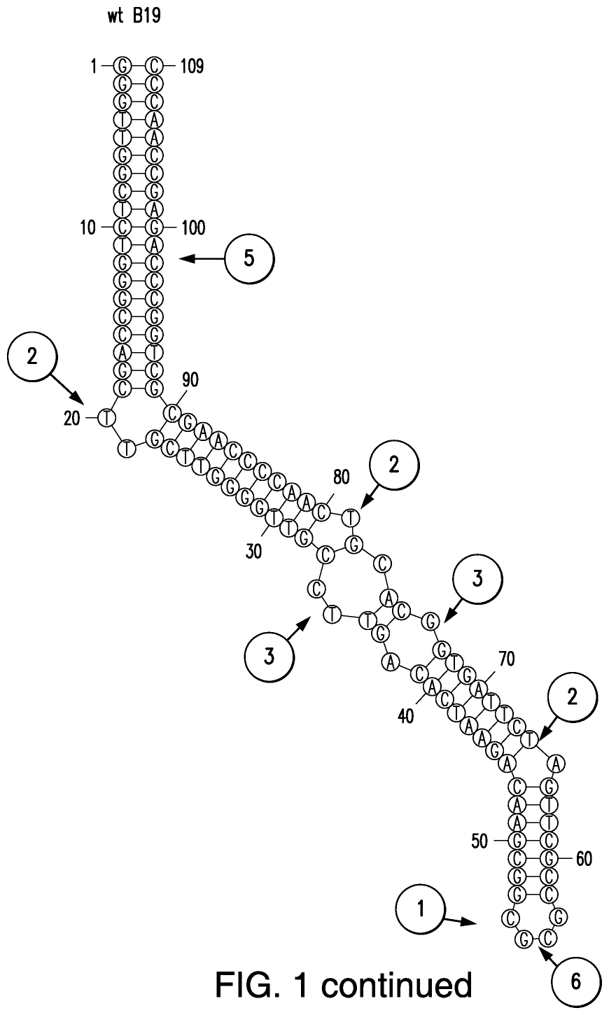

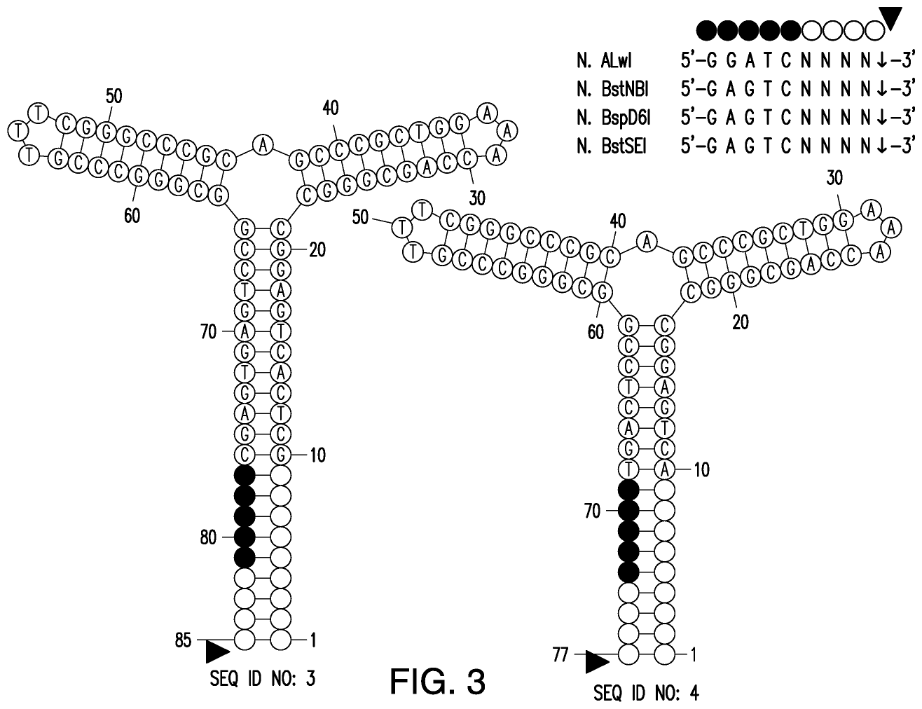

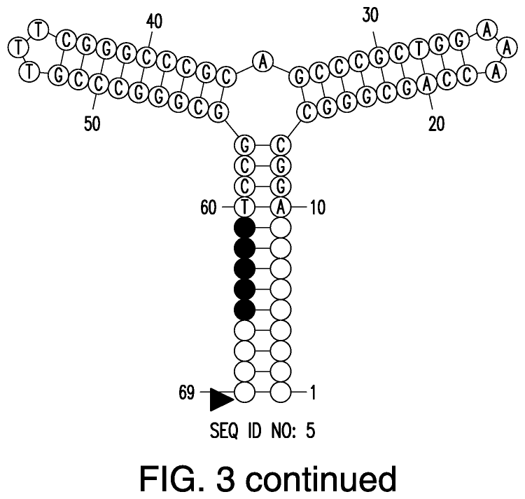

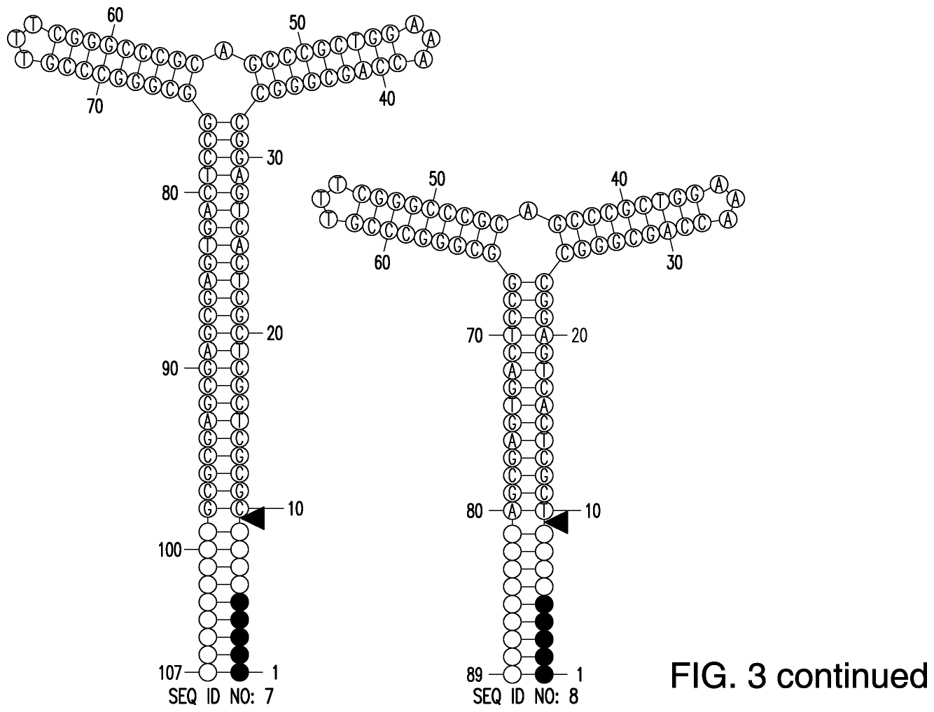

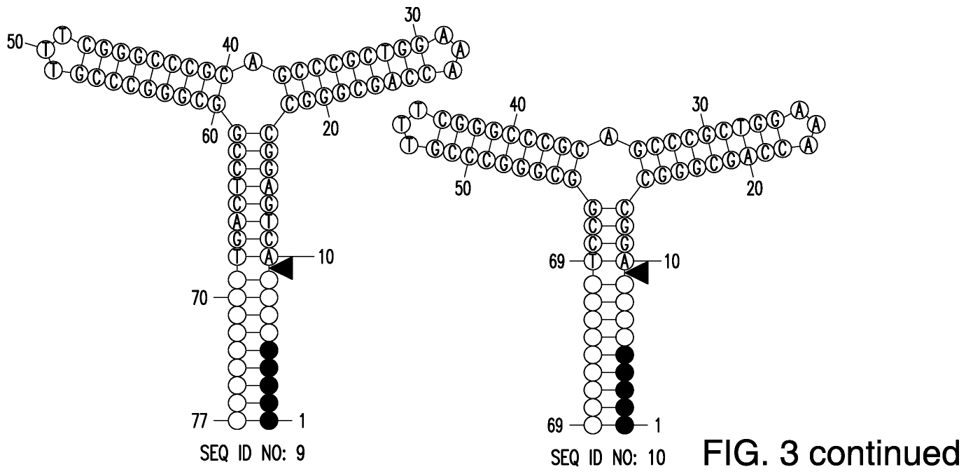

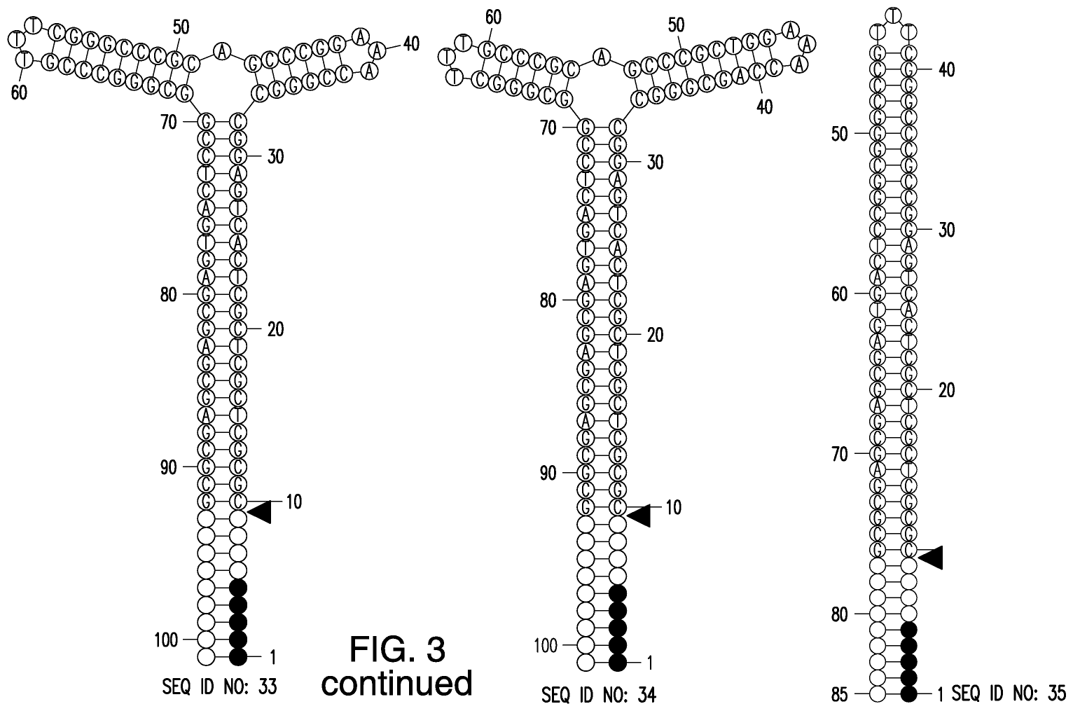

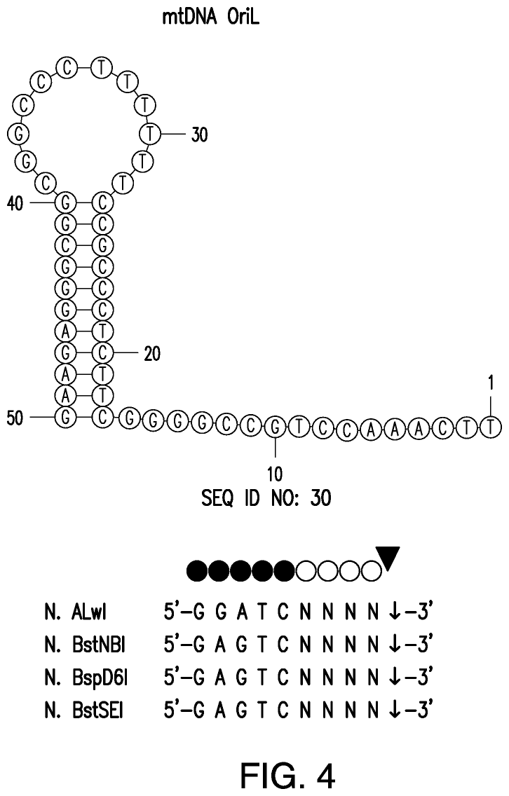

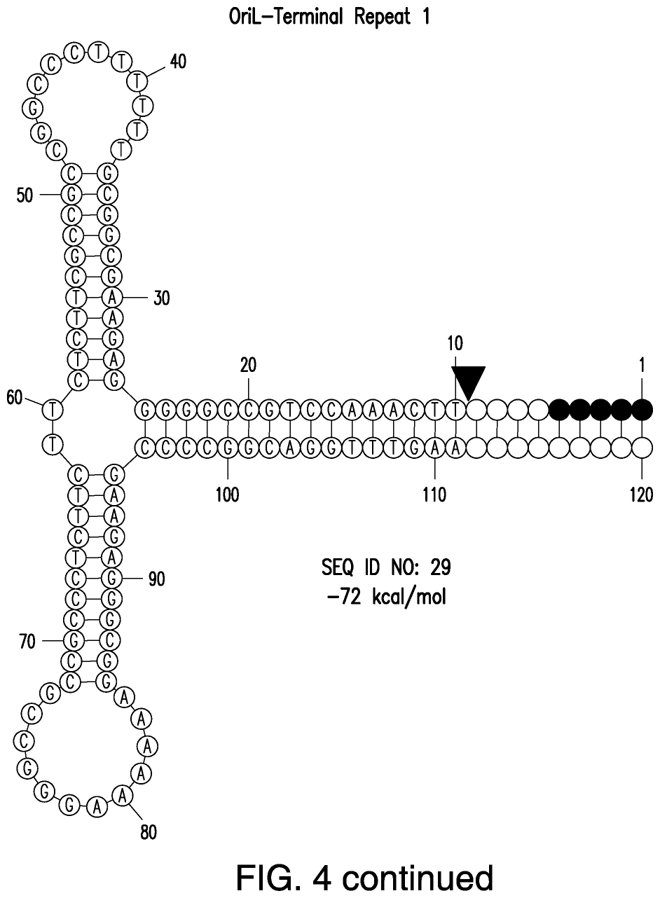

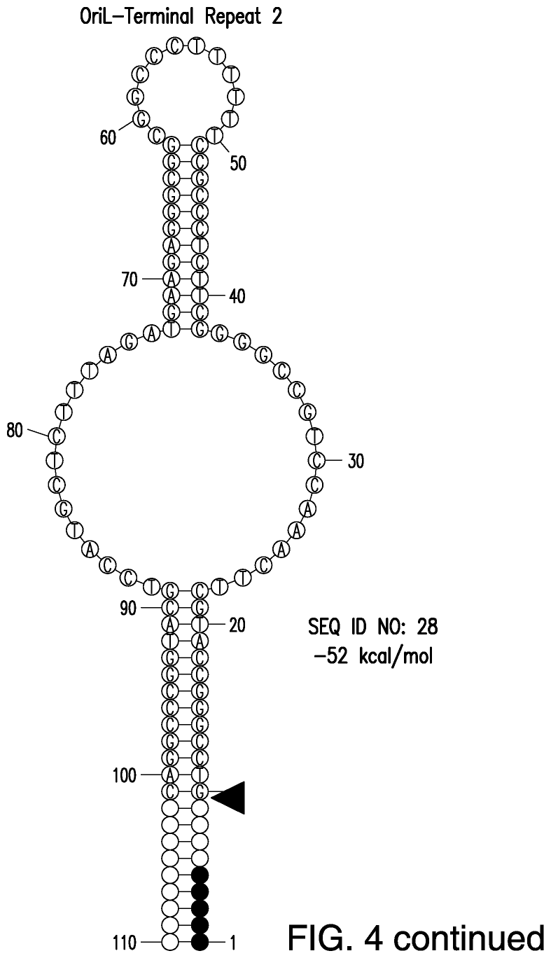

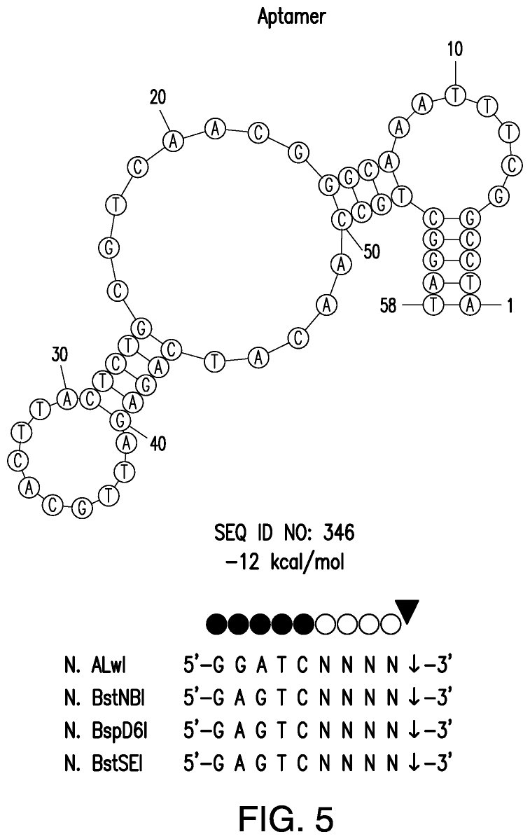

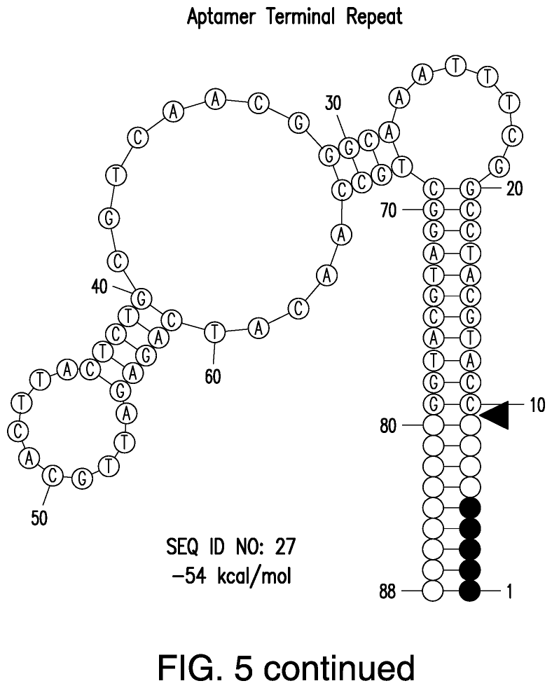

TABLE 3
Sequences of ssDNA overhang and the corresponding
structure after annealing.

| ssDNA overhang sequences | Structures after annealing |
|---|---|
| SEQ ID NO: 3 | FIG. 3 |
| SEQ ID NO: 4 | FIG. 3 |
| SEQ ID NO: 5 | FIG. 3 |
| SEQ ID NO: 7 | FIG. 3 |
| SEQ ID NO: 8 | FIG. 3 |
| SEQ ID NO: 9 | FIG. 3 |
| SEQ ID NO: 10 | FIG. 3 |
| SEQ ID NO: 33 | FIG. 3 |
| SEQ ID NO: 34 | FIG. 3 |
| SEQ ID NO: 35 | FIG. 3 |
| SEQ ID NO: 27 | FIG. 5 |
| SEQ ID NO: 29 | FIG. 4 |
| SEQ ID NO: 28 | FIG. 4 |
| SEQ ID NO. 183 (HBOV derived) | FIG. 1 |
| SEQ ID NO. 184 (B19 derived) | FIG. 1 |

In some embodiments, the structure of the DNA molecules provided herein is the same after 2, 3, 4, 5, 10 or 20 cycles of denaturing/renaturing (e.g. denaturing as described in Section 5.3.3 and re-annealing as described in this Section (Section 5.3.5)). DNA structures can be described by an ensemble of structures at or around the energy minimum. In certain embodiments, the ensemble DNA structure is the same after 2, 3, 4, 5, 10 or 20 cycles of denaturing/renaturing. In one embodiment, the folded hairpin structure formed from the ITR, or IR provided herein is the same after 2, 3, 4, 5, 10 or 20 cycles of denaturing/renaturing. In another embodiment, the ensemble structure of the folded hairpin is the same after 2, 3, 4, 5, 10 or 20 cycles of denaturing/renaturing.

5.3.6 Incubating with Exonuclease

The disclosure provides a step of incubating with an exonuclease as described in Section 3. Exonucleases cleaves nucleotides from the end (exo) of a DNA molecule. Exonucleases can cleave nucleotides along the 5′ to 3′ direction, along the 3′ to 5′ direction, or along both directions. In certain embodiments, an exonuclease for use in the methods provided herein cleaves nucleotides with no sequence specificity. In some embodiments, an exonuclease for use in the methods provided herein digests the DNA fragments comprising ends created by one or more nicking endonuclease recognizing and cutting the fifth and sixth restriction sites or by restriction enzyme cleaving the plasmid or a fragment of the plasmid, as provided in Section 5.4.6.

Various exonucleases known and used in the art can be used in the methods provided herein. An exemplary list of exonucleases provided as embodiments for the restriction enzymes for use in the methods are described in the catalog of New England Biolabs, which is available at neb.com/products/dna-modifying-enzymes-and-cloning-technologies/nucleases and incorporated herein in its entirety by reference. The conditions for the various exonucleases to digest the DNA molecules are known for the various exonucleases provided herein, including the temperatures, the salt concentration, the pH, the buffering reagent, the presence or absence of certain detergent, and the duration of incubation to achieve the desired percentage of digestion. These conditions are readily available from the websites or catalogs of various vendors of the restriction enzymes, e.g., New England BioLabs. The disclosure provides that the step of incubating the DNA molecule with the restriction enzymes is performed according to the incubation conditions as known and practiced in the art.

The step of incubating exonucleases selectively digests the DNA molecules with one or more ends, while leaving the hairpin-ended DNA molecules intact. As is clear from the description of Sections 5.3.5 and 5.5, the hairpin-ended DNA molecules comprise 0, 1, 2, or more nicks. In some embodiments, an exonuclease for use in the methods provided herein can be an exonuclease that selectively digests DNA molecules with one or more ends, while leaving intact the circular ssDNA/dsDNA molecules or DNA molecules comprising one or more nicks but no ends. In one embodiment, an exonuclease for use in the methods provided herein can be Exonuclease V (RecBCD). In one embodiment, an exonuclease for use in the methods provided herein can be Exonuclease VIII or truncated Exonuclease VIII. Exonuclease V (RecBCD), Exonuclease VIII, and truncated Exonuclease VIII comprise the selectivity described in this paragraph. In certain embodiments, an exonuclease for use in the methods provided herein can be an exonuclease that selectively digests linear segments of DNA molecules, initiating from one or more nicks, but cannot progress to digest folded hairpins, terminating the digestion at the hairpin and leaving a ssDNA behind. In certain embodiments, an exonuclease for use to initiate at one or more nicks and/or double strand break can be a T7 exonuclease. Other suitable exonucleases are also known, used in the art, and provided herein, for example, as described on the websites or in the catalogs of various vendors of exonucleases including New England BioLabs.

In some embodiments, after exonuclease treatment, the DNA molecules of the present disclosure are substantially free of any prokaryotic backbone sequences. In some embodiments, the backbone refers to the plasmid sequence that is not part of the sequence encompassing the expression cassette in between the two ITRs. In some embodiments, the backbone refers to the vector sequence that is not part of the sequence encompassing the expression cassette in between the two ITRs. In some embodiments, the isolated DNA molecules of the disclosure are 100% free, 99% free, 98% free, 97% free, 96% free, 95% free, 94% free, 93% free, 92% free, 91% free, or 90% free of prokaryotic backbone sequence of the parental plasmid.

5.3.7 Repairing the Nicks with a Ligase

The disclosure provides an optional step of repairing the nicks with a ligase as described in Section 3. DNA ligases catalyze the joining of two ends of DNA molecules by forming one or more new covalent bonds. For example, commonly used T4 DNA ligase catalyzes the formation of a phosphodiester bond between juxtaposed 5′ phosphate and 3′ hydroxyl termini in DNA. The formation of new covalent bonds that are catalyzed by ligase to joint two DNA molecules is referred to as “ligation.” In certain embodiments, a DNA ligase for use in the methods provided herein ligates nucleotides with no sequence specificity. In some embodiments, a DNA ligase for use in the methods provided herein ligates the two ends at one nick of the DNA molecule described in Section 5.5, thereby repairing said one nick. In some embodiments, a DNA ligase for use in the methods provided herein ligates each pair of two ends at the two nicks of the DNA molecule described in Section 5.5, thereby repairing the two nicks. In some embodiments, a DNA ligase for use in the methods provided herein ligates each pair of two ends at all nicks of the DNA molecule described in Section 5.5, thereby repairing all nicks of the DNA molecule. When the DNA molecule described in Section 5.5 forms a circular DNA after all nicks of the DNA molecule described in Section 5.5 have been repaired. As described in Section 5.5, in some embodiments, the DNA molecule described in Section 5.5 consists of two nicks. In certain embodiments, the DNA molecule described in Section 5.5 comprises two nicks. In other embodiments, the DNA molecule described in Section 5.5 consists of one nick. In yet other embodiments, the DNA molecule described in Section 5.5 comprises one nick.

In some embodiments, the step of repairing the nicks with a ligase can be performed according to the incubation conditions as known and practiced in the art.

Various ligases known and used in the art can be used in the methods provided herein. An exemplary list of ligases provided as embodiments for the ligases for use in the methods are described in the catalog of New England Biolabs, which is available at neb.com/products/dna-modifying-enzymes-and-cloning-technologies/dna-ligases/dna-ligases and incorporated herein in its entirety by reference. The conditions for the various ligases to digest the DNA molecules are known for the various ligases provided herein, including the temperatures, the salt concentration, the pH, the buffering reagent, the presence or absence of certain detergent, and the duration of incubation to achieve the desired percentage of digestion. These conditions are readily available from the websites or catalogs of various vendors of the restriction enzymes, e.g., New England BioLabs. The ligation conditions also correlate with the freedom of movement of the two DNA ends to be ligated. When the two DNA ends can be brought to proximity or can have a higher probability of coming to proximity of each other, for example by both ends annealing to a common DNA strand, ligation can be enhanced. In one embodiment, the method step provided in this Section (Section 5.3.7) repairs the nicks with a ligase to form a circular DNA, wherein the two DNA ends at any nick of the DNA molecule described in Section 5.5 have annealed to a common DNA strand. In some embodiments, the step of repairing the nicks with a ligase is performed according to the incubation conditions as known and practiced in the art.

In certain embodiments, the methods provided in this Section 5.2 can be used to generate the hairpin-ended DNA molecules described herein at high scale, high yield, and/or high purity. In certain embodiments, high scale, high yield, and/or high purity can be accomplished in a single reaction vessel. In certain embodiments, the high scale is at least 1 mg, 10 mg, 100 mg, 1 g, 10 g, 100 g, 1 kg, or at least 10 kg. In certain embodiment, the high yield is at least 50%, 60%, 70%, 80%, 90%, 95%, 98%, or at least 99% yield (comparing number of plasmid copies used as input and number of hairpin-ended DNA molecules as product). In certain embodiments, the high purity is at least 50%, 60%, 70%, 80%, 90%, 95%, 98%, or at least 99% purity of hairpin-ended DNA molecules as product as a result of a method provided herein.

### 5.4 DNA Molecules Used in the Methods

The disclosure provides various aspects and embodiments of the DNA molecules for use in the methods provided herein as described in Section 5.3 above. In one aspect, provided herein is a double-stranded DNA molecule comprising in 5′ to 3′ direction of the top strand: i) a first inverted repeat (e.g. as described in Section 5.4.1), wherein a first and a second restriction site for nicking endonuclease are arranged on opposite strands in proximity of the first inverted repeat such that nicking results in a top strand 5′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette (e.g. as described in Section 5.4.3); and iii) a second inverted repeat (e.g. as described in Section 5.4.1), wherein a third and a fourth restriction site for nicking endonuclease are arranged on opposite strands in proximity of the second inverted repeat such that nicking results in a top strand 3′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2). In certain embodiments, the top strand 5′ overhang comprises the first inverted repeat. In certain embodiments, the top strand 3′ overhang comprises the second inverted repeat. In certain embodiments, the top strand 5′ overhang comprises the first inverted repeat and the top strand 3′ overhang comprises the second inverted repeat.

In another aspect, provided herein is a double strand DNA molecule comprising in 5′ to 3′ direction of the top strand: i) a first inverted repeat (e.g. as described in Section 5.4.1), wherein a first and a second restriction site for nicking endonuclease are arranged on opposite strands in proximity of the first inverted repeat such that nicking results in a bottom strand 3′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette (e.g. as described in Section 5.4.3); and iii) a second inverted repeat (e.g. as described in Section 5.4.1), wherein a third and a fourth restriction site for nicking endonuclease are arranged on opposite strands in proximity of the second inverted repeat such that nicking results in a bottom strand 5′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2 or depicted in FIGS. 2A and 2C). In certain embodiments, the bottom strand 3′ overhang comprises the first inverted repeat. In certain embodiments, the bottom strand 5′ overhang comprises the second inverted repeat. In certain embodiments, the bottom strand 3′ overhang comprises the first inverted repeat and the bottom strand 5′ overhang comprises the second inverted repeat.

In yet another aspect, provided herein is a double-stranded DNA molecule comprising in 5′ to 3′ direction of the top strand: i) a first inverted repeat (e.g. as described in Section 5.4.1), wherein a first and a second restriction site for nicking endonuclease are arranged on opposite strands in proximity of the first inverted repeat such that nicking results in a top strand 5′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette (e.g. as described in Section 5.4.3); and iii) a second inverted repeat (e.g. as described in Section 5.4.1), wherein a third and a fourth restriction site for nicking endonuclease are arranged on opposite strands in proximity of the second inverted repeat such that nicking results in a bottom strand 5′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2). In certain embodiments, the top strand 5′ overhang comprises the first inverted repeat. In certain embodiments, the bottom strand 5′ overhang comprises the second inverted repeat. In certain embodiments, the top strand 5′ overhang comprises the first inverted repeat and the bottom strand 5′ overhang comprises the second inverted repeat.

In a further aspect, provide herein is a double strand DNA molecule comprising in 5′ to 3′ direction of the top strand: i) a first inverted repeat (e.g. as described in Section 5.4.1), wherein a first and a second restriction site for nicking endonuclease are arranged on opposite strands in proximity of the first inverted repeat such that nicking results in a bottom strand 3′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette (e.g. as described in Section 5.4.3); and iii) a second inverted repeat (e.g. as described in Section 5.4.1), wherein a third and a fourth restriction site for nicking endonuclease are arranged on opposite strands in proximity of the second inverted repeat or a fragment thereof (e.g., at least 50%, 60%, 70%, 75%, 80%, 85%, 90%, 95%, or 99% of the second inverted repeat) such that nicking results in a top strand 3′ overhang comprising the second inverted repeat upon separation of the top from the bottom strand of the second inverted repeat (e.g. as described in Sections 5.3.3, 5.3.4 and 5.4.2 or depicted in FIGS. 2B and 2C). In certain embodiments, the bottom strand 3′ overhang comprises the first inverted repeat. In certain embodiments, the top strand 3′ overhang comprises the second inverted repeat. In certain embodiments, the bottom strand 3′ overhang comprises the first inverted repeat and the top strand 3′ overhang comprises the second inverted repeat.

In one aspect, provided herein is a double-stranded DNA molecule comprising in 5′ to 3′ direction of the top strand: i) a first inverted repeat (e.g. as described in Section 5.4.1), wherein a first and a second target site for the guide nucleic acids for programmable nicking enzyme are arranged on opposite strands in proximity of the first inverted repeat such that nicking by programmable nicking enzyme results in a top strand 5′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette (e.g. as described in Section 5.4.3); and iii) a second inverted repeat (e.g. as described in Section 5.4.1), wherein a third and a fourth target site for the guide nucleic acids for programmable nicking enzyme are arranged on opposite strands in proximity of the second inverted repeat such that nicking by programmable nicking enzyme results in a top strand 3′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2). In certain embodiments, the top strand 5′ overhang comprises the first inverted repeat. In certain embodiments, the top strand 3′ overhang comprises the second inverted repeat. In certain embodiments, the top strand 5′ overhang comprises the first inverted repeat and the top strand 3′ overhang comprises the second inverted repeat.

In another aspect, provided herein is a double strand DNA molecule comprising in 5′ to 3′ direction of the top strand: i) a first inverted repeat (e.g. as described in Section 5.4.1), wherein a first and a second target site for the guide nucleic acids for programmable nicking enzyme are arranged on opposite strands in proximity of the first inverted repeat such that nicking by programmable nicking enzyme results in a bottom strand 3′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette (e.g. as described in Section 5.4.3); and iii) a second inverted repeat (e.g. as described in Section 5.4.1), wherein a third and a fourth target site for the guide nucleic acids for programmable nicking enzyme are arranged on opposite strands in proximity of the second inverted repeat such that nicking by programmable nicking enzyme results in a bottom strand 5′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2 or depicted in FIGS. 2A and 2C). In certain embodiments, the bottom strand 3′ overhang comprises the first inverted repeat. In certain embodiments, the bottom strand 5′ overhang comprises the second inverted repeat. In certain embodiments, the bottom strand 3′ overhang comprises the first inverted repeat and the bottom strand 5′ overhang comprises the second inverted repeat.

In yet another aspect, provided herein is a double-stranded DNA molecule comprising in 5′ to 3′ direction of the top strand: i) a first inverted repeat (e.g. as described in Section 5.4.1), wherein a first and a second target site for the guide nucleic acids for programmable nicking enzyme are arranged on opposite strands in proximity of the first inverted repeat such that nicking by programmable nicking enzyme results in a top strand 5′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette (e.g. as described in Section 5.4.3); and iii) a second inverted repeat (e.g. as described in Section 5.4.1), wherein a third and a fourth target site for the guide nucleic acids for programmable nicking enzyme are arranged on opposite strands in proximity of the second inverted repeat such that nicking by programmable nicking enzyme results in a bottom strand 5′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2). In certain embodiments, the top strand 5′ overhang comprises the first inverted repeat. In certain embodiments, the bottom strand 5′ overhang comprises the second inverted repeat. In certain embodiments, the top strand 5′ overhang comprises the first inverted repeat and the bottom strand 5′ overhang comprises the second inverted repeat.

In a further aspect, provide herein is a double strand DNA molecule comprising in 5′ to 3′ direction of the top strand: i) a first inverted repeat (e.g. as described in Section 5.4.1), wherein a first and a second target site for the guide nucleic acids for programmable nicking enzyme are arranged on opposite strands in proximity of the first inverted repeat such that nicking by programmable nicking enzyme results in a bottom strand 3′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette (e.g. as described in Section 5.4.3); and iii) a second inverted repeat (e.g. as described in Section 5.4.1), wherein a third and a fourth target site for the guide nucleic acids for programmable nicking enzyme are arranged on opposite strands in proximity of the second inverted repeat such that nicking by programmable nicking enzyme results in a top strand 3′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2 or depicted in FIGS. 2B and 2C). In certain embodiments, the bottom strand 3′ overhang comprises the first inverted repeat. In certain embodiments, the top strand 3′ overhang comprises the second inverted repeat. In certain embodiments, the bottom strand 3′ overhang comprises the first inverted repeat and the top strand 3′ overhang comprises the second inverted repeat.

The DNA molecules provided herein comprise various features or have various embodiments as described in Section 3 and the preceding paragraphs of this Section (Section 5.4), which features and embodiments are further described in the various subsections below: the embodiments for the inverted repeats, including the first inverted repeat and/or the second inverted repeat, are described in Section 5.4.1, the embodiments for the restriction enzymes, nicking endonucleases, and their respective restriction sites are described in Sections 5.4.2 and 5.3.4, the embodiments for the programmable nicking enzymes and their targeting sites are described in Section 5.3.4, the embodiments for the expression cassette are described in Section 5.4.3, the embodiments for plasmids and vectors are described in Section 5.4.6, the embodiments for DNA molecules comprising less than 4 restriction site for nicking endonucleases are described in Section 5.4.7. As such, the disclosure provides DNA molecules comprising any permutations and combinations of the various embodiments of DNA molecules and embodiments of features of the DNA molecules described herein. In further embodiments, the arrangement among the ITR, the expression cassette, the restriction sites for nicking endonuclease or restriction enzymes, and the programmable nicking enzyme and their targeting sites can be any arrangement as described in Sections 5.3.3, 5.3.4, 5.3.5, 5.4.1, 5.4.2, 5.4.3 5.4.7, and 5.5.

In one aspect, provided herein is a double-stranded DNA molecule comprising in the 5′ to 3′ direction of the top strand: i) a first viral replication deficient inverted repeat (e.g. as described in Section 5.4.1 and Section 5.4.5), wherein a first and a second restriction site for nicking endonuclease are arranged on opposite strands in proximity of the first inverted repeat such that nicking results in a top strand 5′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4, 5.4.2 and 5.4.5); ii) an expression cassette (e.g. as described in Section 5.4.3); and iii) a second viral replication deficient inverted repeat (e.g. as described in Section 5.4.1 and Section 5.4.5), wherein a third and a fourth restriction site for nicking endonuclease are arranged on opposite strands in proximity of the second inverted repeat such that nicking results in a top strand 3′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2). In certain embodiments, the top strand 5′ overhang comprises the first viral replication deficient inverted repeat. In certain embodiments, the top strand 3′ overhang comprises the second viral replication deficient inverted repeat. In certain embodiments, the top strand 5′ overhang comprises the first viral replication deficient inverted repeat and the top strand 3′ overhang comprises the second viral replication deficient inverted repeat.

In another aspect, provided herein is a double strand DNA molecule comprising in the 5′ to 3′ direction of the top strand: i) a first viral replication deficient inverted repeat (e.g. as described in Section 5.4.1 and 5.4.5), wherein a first and a second restriction site for nicking endonuclease are arranged on opposite strands in proximity of the first inverted repeat such that nicking results in a as described in Sections 5.3.3, 5.3.4 and 5.4.2); ii) an expression cassette (e.g. as described in Section 5.4.3); and iii) a second viral replication deficient inverted repeat (e.g. as described in Section 5.4.1 and Section 5.4.5), wherein a third and a fourth restriction site for nicking endonuclease are arranged on opposite strands in proximity of the second inverted repeat such that nicking results in a bottom strand 5′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2 or depicted in FIGS. 2A and 2C). In certain embodiments, the bottom strand 3′ overhang comprises the first viral replication deficient inverted repeat. In certain embodiments, the bottom strand 5′ overhang comprises the second viral replication deficient inverted repeat. In certain embodiments, the bottom strand 3′ overhang comprises the first viral replication deficient inverted repeat and the bottom strand 5′ overhang comprises the second viral replication deficient inverted repeat.

In yet another aspect, provided herein is a double-stranded DNA molecule comprising in the 5′ to 3′ direction of the top strand: i) a first viral replication deficient inverted repeat (e.g. as described in Section 5.4.1 and 5.4.5), wherein a first and a second restriction site for nicking endonuclease are arranged on opposite strands in proximity of the first inverted repeat such that nicking results in a top strand 5′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette (e.g. as described in Section 5.4.3); and iii) a second viral replication deficient inverted repeat (e.g. as described in Section 5.4.1 and 5.4.5), wherein a third and a fourth restriction site for nicking endonuclease are arranged on opposite strands in proximity of the second inverted repeat such that nicking results in a bottom strand 5′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2). In certain embodiments, the top strand 5′ overhang comprises the first viral replication deficient inverted repeat. In certain embodiments, the bottom strand 5′ overhang comprises the second viral replication deficient inverted repeat. In certain embodiments, the top strand 5′ overhang comprises the first viral replication deficient inverted repeat and the bottom strand 5′ overhang comprises the second viral replication deficient inverted repeat.

In a further aspect, provide herein is a double stranded DNA molecule comprising in 5′ to 3′ direction of the top strand: i) a first viral replication deficient inverted repeat (e.g. as described in Section 5.4.1 and 5.4.5), wherein a first and a second restriction site for nicking endonuclease are arranged on opposite strands in proximity of the first inverted repeat such that nicking results in a bottom strand as described in Sections 5.3.3, 5.3.4 and 5.4.2); ii) an expression cassette (e.g. as described in Section 5.4.3); and iii) a second viral replication deficient inverted repeat (e.g. as described in Section 5.4.1 and Section 5.4.5), wherein a third and a fourth restriction site for nicking endonuclease are arranged on opposite strands in proximity of the second inverted repeat or a fragment thereof (e.g., at least 50%, 60%, 70%, 75%, 80%, 85%, 90%, 95%, or 99% of the second inverted repeat) such that nicking results in a top strand 3′ overhang comprising the second inverted repeat upon separation of the top from the bottom strand of the second inverted repeat (e.g. as described in Sections 5.3.3, 5.3.4 and 5.4.2 or depicted in FIGS. 2B and 2C). In certain embodiments, the bottom strand 3′ overhang comprises the first viral replication deficient inverted repeat. In certain embodiments, the top strand 3′ overhang comprises the second viral replication deficient inverted repeat. In certain embodiments, the bottom strand 3′ overhang comprises the first viral replication deficient inverted repeat and the top strand 3′ overhang comprises the second viral replication deficient inverted repeat.

The DNA molecule provided herein can be a DNA molecule in its native environment or an isolated DNA molecule. In certain embodiments, the DNA molecule is a DNA molecule in its native environment. In some embodiments, the DNA molecule is an isolated DNA molecule. In one embodiment, the isolated DNA molecule can be a DNA molecule of at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% purity. In another embodiment, the isolated DNA molecule can be a DNA molecule of about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% purity. Other embodiments of the isolated DNA molecules provided herein in terms of purities are further described in Section 5.4.8, which can be combined in any suitable combination with the embodiments provided in this paragraph.

As the DNA molecules can be fully engineered (e.g. synthetically produced or recombinantly produced), the DNA molecules provided herein including those of Sections 3 and this Section 5.4 can lack certain sequences or features as further described in Section 5.4.5.

### 5.4.1 Inverted Repeats

The ITRs or IRs provided in Sections 3 and this Section (Section 5.4.1) can form the hairpinned ITRs in the hairpin-ended DNA molecules provided in Section 5.5, for example upon performing the method steps described in Sections 3, 5.3.3, 5.3.4, and 5.3.5. Accordingly, in some embodiments, the ITRs or IRs provided in Sections 3 and this Section (Section 5.4.1) can comprise any embodiments of the IRs or ITRs provided in Sections 3 and Section 5.5 and additional embodiments provided in this Section (Section 5.4.1), in any combination.

The majority of DNA in the cells comprises two strands engaged in Watson-Crick base pairing, which sequesters most of the functional groups and limits structural and functional diversity. While this is a desirable property for a molecule whose function is to store genetic information, single stranded viruses have evolved to utilize the intramolecular interaction of linear single stranded DNA (ssDNA) to form secondary structures that add another layer of functional complexity. One major contributing factor is that these ssDNA viral genomes are composed of just one strand that folds back on itself to form hairpins.

The secondary structure of a single stranded DNA molecule can be a representation of the pattern, based on an initial DNA sequence, of complementary base-pairings that are formed between the constituent nucleotides. The sequence, represented as a string of four letters (one for each nucleotide species), is a single strand consisting of the nucleotides which are generally assumed to form different secondary structures with minimum free energies that are governed by thermodynamic interactions.

“Inverted repeat” or “IR” refers to a single stranded nucleic acid sequence that comprises a palindromic sequence region. This palindromic region comprises a sequence of nucleotides as well as its reverse complement, i.e., “palindromic sequence” as further described below, on the same strand as further described below. In a denatured state, meaning in conditions in which the hydrophobic stacking attractions between the bases are broken, the IR nucleic acid sequence is present in a random coil state (e.g., at high temperature, presence of chemical agents, high pH, etc.). As conditions become more physiological, said IR can fold into a secondary structure whose outermost regions are non-covalently held together by base pairing. In some embodiments, an IR can be an ITR. In certain embodiments, an IR comprise an ITR. In some embodiments an IR can be a hairpinned inverted repeat. In certain embodiments, an inverted repeat, once folded upon itself, comprises at least one hairpin loop (also known as stem loop) in which an unpaired loop of single stranded DNA is created when the DNA strand folds and forms base pairs with another section of the same strand. In certain embodiments, an inverted repeat can comprise one, two, three, four, five, six, seven, eight, nine, or ten such hairpin loop structures.

“Inverted terminal repeat” “terminal repeat,” “TR,” or “ITR” refers to an inverted repeat region that is at or proximal to a terminal of a single strand DNA molecule or an inverted repeat that is at or in the single strand overhang of a dsDNA molecule. An ITR can fold onto itself as a result of the palindromic sequence in the ITR. In one embodiment, an ITR is at or proximal to one end of an ssDNA. In another embodiment, an ITR is at or proximal to one end of a dsDNA. In yet another embodiment, two ITRs are each at or proximal to the two respective ends of an ssDNA. In a further embodiment, two ITRs are each at or proximal to the two respective ends of a dsDNA. In some embodiments, the non-ITR part of the ssDNA or dsDNA is heterologous to the ITR. In certain embodiments, the non-ITR part of the ssDNA or dsDNA is homologous to the ITR. In a denatured state, meaning in conditions in which the hydrophobic stacking attractions between the bases are broken, the ITR comprising nucleic acid sequence is present in a random coil state (e.g., at high temperature, presence of chemical agents, high pH, etc.). In some embodiments, as conditions become more suitable for annealing as described in Section 5.3.5, the ITR can fold on itself into a structure that is non-covalently held together by base pairing while the heterologous non-ITR part of the dsDNA remain intact or the heterologous non-ITR part of the ssDNA molecule can hybridize with a second ssDNA molecule comprising the reverse complement sequence of the heterologous DNA molecule. The resulting complex of two hybridized DNA strands encompass three distinct regions, a first folded single stranded ITR covalently linked to a double stranded DNA region that is in turn covalently linked to a second folded single stranded ITR. In certain embodiments, the ITR sequence can start at one of the restriction site for nicking endonuclease described in Sections 3, 5.3.4, and 5.4.2 and end at the last base before the dsDNA. In one embodiment, as opposed to a linear double stranded DNA molecule, the ITR present at the 5′ and 3′ termini of the top and bottom strand at either end of the DNA molecule can fold in and face each other (e.g., 3′ to 5′, 5′ to 3′ or vice versa) and therefore do not expose a free 5′ or 3′ terminus at either end of the nucleic acid duplex. When the ITR folds on itself, the dsDNA in the folded ITR can be immediately next to the dsDNA of the non-ITR part of the DNA molecule, creating a nick flanked by dsDNA in some embodiments, or the dsDNA in the folded ITR can be one or more nucleotide apart from the dsDNA of the non-ITR part of the DNA molecule, creating a “ssDNA gap” flanked by dsDNA in other embodiments. The two ITRs that flank the non-ITR DNA sequence are referred to an “ITR pair”. In some embodiments, when the ITR assumes its folded state, it is resistant to exonuclease digestion (e.g., exonuclease V), e.g. for over an hour at 37° C.

The boundary between the terminal base of the ITR folded into its secondary structure and the terminal base of the DNA hybridized duplex can further be stabilized by stacking interactions (e.g., coaxial stacking) between base pairs flanking the nick or ssDNA gap and these interactions are sequence dependent. In the case of a structure resembling a nick, an equilibrium between two conformations can exist wherein, the first conformation is very close to that of the intact double helix where stacking between the base pairs flanking the nick is conserved while the other conformation corresponds to complete loss of stacking at the nick site thus inducing a kink in DNA. Nicked molecules are known to move somewhat slower during polyacrylamide and agarose gel electrophoresis than intact molecules of the same size. In some cases, this retardation is enhanced at higher temperatures. It is thought that the fast equilibration between stacked/straight and unstacked/bent conformations of the nick directly affects the mobility of DNA molecule during gel electrophoreses, leading to differential retardation characteristic to a DNA molecule carrying the nick.

Without being bound by theory, it is thought that cellular proteins can recognize parallel 5′ and 3′ termini as double strand breaks and can engage as well as process these, which can adversely affect the fate of the DNA in a cell. Hence, the ITR can prevent premature, unwanted degradation of the expression cassette with ITRs at one or both of its two ends as provided in Sections 3 and 5.5 and this Section (Section 5.4.1).

By placing a first and a second restriction site for nicking endonucleases on opposite strands and in proximity of the inverted repeats and subsequent separation of the top from the bottom strand of the inverted repeat, the resulting overhang can fold back on itself and form a double stranded end that contains at least one restriction site for the nicking endonuclease. In some embodiments, the folded ITR resembles the secondary structure conformation of viral ITRs. In one embodiment, the ITR is located on both the 5′ and 3′ terminus of the bottom strand (e.g. a left ITR and right ITR). In another embodiment, the ITR is located on both the 5′ and 3′ terminus of the top strand. In yet another embodiment, one ITR is located at the 5′ terminus of the top strand, and the other ITR is located at the opposite end of the bottom strand (e.g. the left ITR at the 5′ terminus on the top strand and the right ITR at the 5′ terminus of the bottom). In yet another embodiment, one ITR is located at the 3′ terminus of the top strand, and the other ITR is located at the 3′ terminus of the bottom strand.

In some aspects, the disclosure provides a DNA molecule comprising palindromic sequences. “Palindromic sequences” or “palindromes” are self-complimentary DNA sequences that can fold back to form a stretch of dsDNA in the self-complimentary region under a condition that favors intramolecular annealing. In some embodiments, a palindromic sequence comprises a contiguous stretch of polynucleotides that is identical when read forwards as when read backwards on the complementary strand. In one embodiment, a palindromic sequence comprises a stretch of polynucleotides that is identical when read forwards as when read backwards on the complementary strand, wherein such stretch is interrupted by one or more stretches of non-palindromic polynucleotides. In another embodiment, a palindromic sequence comprises a stretch of polynucleotides that is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical when read forwards as when read backwards on the complementary strand. In yet another embodiment, a palindromic sequence comprises a stretch of polynucleotides that is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical when read forwards as when read backwards on the complementary strand, wherein such stretch is interrupted by one or more stretches of non-palindromic polynucleotides. In some specific embodiments, provided herein is a double stranded DNA molecule having a first and a second restriction site for nicking endonucleases on opposite strands of the double strand DNA, wherein, after nicking and separating the top from the bottom strand of the inverted repeat, the resulting inverted repeat comprises, between said first and second restriction sites, a palindromic sequence that is 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical when read forwards as when read backwards on the complementary strand. An ssDNA comprising one or more palindromic sequences can fold back upon itself, to form double stranded base pairs comprising a secondary structure (e.g., a hairpin loop, or a three-way junction).

Under appropriate conditions, for example as described in Sections 5.3.3, 5.3.4, and 5.3.5, An IR or an ITR provided in this Section (Section 5.4.1) can fold and form hairpin structures as described in this Section (Section 5.4.1) and Section 5.5, including stems, a primary stem, loops, turning points, bulges, branches, branch loops, internal loops, and/or any combination or permutation of the structural features described in Section 5.5.

In one embodiment, an IR or ITR for the methods and compositions provided herein comprises one or more palindromic sequences. In some embodiments, an IR or ITR described herein comprises palindromic sequences or domains that in addition to forming the primary stem domain can form branched hairpin structures. In some embodiments, an IR or ITR comprises palindromic sequences that can form any number of branched hairpins. In certain specific embodiments, an IR or ITR comprises palindromic sequences that can form 1 to 30, or any subranges of 1 to 30, branched hairpins. In some specific embodiments, an IR or ITR comprises palindromic sequences that can form 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 branched hairpins. In some embodiments, an IR or ITR comprises sequence that can form two branched hairpin structures that lead to a three-way junction domain (T-shaped). In some embodiments, an IR or ITR comprises sequence that can form three branched hairpin structures that lead to a four-way junction domain (or cruciform structure). In some embodiments, an IR or ITR comprises sequence that can form a non-T-shaped hairpin structure, e.g., a U-shaped hairpin structure. In some embodiments, an IR or ITR comprises sequence that can form interrupted U-shaped hairpin structure including a series of bulges and base pair mismatches. In some embodiments, the branched hairpins all have the same length of stem and/or loop. In some embodiments, one branched hairpin is smaller (e.g. truncated) than the other branched hairpins. Some exemplar embodiments of the hairpin structures and the structural elements of the hairpin structures are depicted in FIG. 1.

“Hairpin closing base pair” refers to the first base pair following the unpaired loop sequence. Certain stem loop sequences have preferred closing base pairs (e.g. GC in AAV2 ITRs). In one embodiment, the stem loop sequence comprises G-C pair as the closing base pair. In another embodiment, the stem loop sequence comprises C-G pair as the closing base pair.

“ITR closing base pair” refers to the first and last nucleotide that forms a base pair in a folded ITR. The terminal base pair is usually the pair of nucleotides of the primary stem domain that are most proximal to the non-ITR sequences (e.g. expression cassette) of the DNA molecule. The ITR closing base pair can be any type of base pair (e.g. CG, AT, GC, or TA). In one embodiment, the ITR closing base pair is a G-C base pair. In another embodiment, the ITR closing base pair is an A-T base pair. In yet another embodiment, the ITR closing base pair is a C-G base pair. In a further embodiment, the ITR closing base pair is a T-A base pair.

The disclosure provides that the DNA secondary structure can be computationally predicted according as known and practiced in the art. DNA secondary structures can be represented in several ways: squiggle plot, graph representation, dot-bracket notation, circular plot, arc diagram, mountain plot, dot plot, etc. In circular plots, the backbone is represented by a circle, and the base pairs are symbolized by arcs in the interior of the circle. In arc diagrams, the DNA backbone is drawn as a straight line and the nucleotides of each base pair are connected by an arc. Both circular and arc plots allow for the identification of secondary structure similarities and differences.

One of the many methods for DNA secondary structure prediction uses the nearest-neighbor model and minimizes the total free energy associated with a DNA structure. The minimum free energy is estimated by summing individual energy contributions from base pair stacking, hairpins, bulges, internal loops, and multi-branch loops. The energy contributions of these elements are sequence- and length-dependent and have been experimentally determined. The segregation of the sequence into a stem loop and sub-stems can be depicted, for example, by displaying the structure as graph plot. In a linear interaction plot, each residue is represented on the abscissa and semi-elliptical lines connect bases that pair with each other (e.g. FIGS. 2A and B).

In some embodiments, the ITR promotes the long-term survival of the nucleic acid molecule in the nucleus of a cell. In some embodiments, the ITR promotes the permanent survival of the nucleic acid molecule in the nucleus of a cell (e.g., for the entire lifespan of the cell). In some embodiments, the ITR promotes the stability of the nucleic acid molecule in the nucleus of a cell. In some embodiments, the ITR inhibits or prevents the degradation of the nucleic acid molecule in the nucleus of a cell.

In certain embodiments, IRs or ITRs can comprise any viral ITR. In other embodiments, IRs or ITRs can comprise a synthetic palindromic sequence that can form a palindrome hairpin structure that does not expose a 5′ or 3′ terminus at the outmost apex or turning point of the repeat.

In some embodiments, the single stranded ITR sequence stretching from one nucleotide of the ITR closing base pair to the other nucleotide of the ITR closing base pair has a Gibbs free energy (ΔG) of unfolding under physiological conditions in the range of −10 kcal/mol to −100 kcal/mol. In one embodiment, the Gibbs free energy (ΔG) of unfolding referred to in the preceding sentence is no more than −10 (meaning ≤−10, including e.g. −20, −30, etc.), no more than −11, no more than −12, no more than −13, no more than −14, no more than −15, no more than −16, no more than −17, no more than −18, no more than −19, no more than −20, no more than −21, no more than −22, no more than −23, no more than −24, no more than −25, no more than −26, no more than −27, no more than −28, no more than −29, no more than −30, no more than −31, no more than −32, no more than −33, no more than −34, no more than −35, no more than −36, no more than −37, no more than −38, no more than −39, no more than −40, no more than −41, no more than −42, no more than −43, no more than −44, no more than −45, no more than −46, no more than −47, no more than −48, no more than −49, no more than −50, no more than −51, no more than −52, no more than −53, no more than −54, no more than −55, no more than −56, no more than −57, no more than −58, no more than −59, no more than −60, no more than −61, no more than −62, no more than −63, no more than −64, no more than −65, no more than −66, no more than −67, no more than −68, no more than −69, no more than −70, no more than −71, no more than −72, no more than −73, no more than −74, no more than −75, no more than −76, no more than −77, no more than −78, no more than −79, no more than −80, no more than −81, no more than −82, no more than −83, no more than −84, no more than −85, no more than −86, no more than −87, no more than −88, no more than −89, no more than −90, no more than −91, no more than −92, no more than −93, no more than −94, no more than −95, no more than −96, no more than −97, no more than −98, no more than −99, or no more than −100 kcal/mol. In another embodiment, the Gibbs free energy (ΔG) of unfolding referred to in the preceding sentence is about −10 (meaning ≤−10, including e.g. −20, −30, etc.), about −11, about −12, about −13, about −14, about −15, about −16, about −17, about −18, about −19, about −20, about −21, about −22, about −23, about −24, about −25, about −26, about −27, about −28, about −29, about −30, about −31, about −32, about −33, about −34, about −35, about −36, about −37, about −38, about −39, about −40, about −41, about −42, about −43, about −44, about −45, about −46, about −47, about −48, about −49, about −50, about −51, about −52, about −53, about −54, about −55, about −56, about −57, about −58, about −59, about −60, about −61, about −62, about −63, about −64, about −65, about −66, about −67, about −68, about −69, about −70, about −71, about −72, about −73, about −74, about −75, about −76, about −77, about −78, about −79, about −80, about −81, about −82, about −83, about −84, about −85, about −86, about −87, about −88, about −89, about −90, about −91, about −92, about −93, about −94, about −95, about −96, about −97, about −98, about −99, or about −100 kcal/mol. In some embodiments, the ITR sequence stretching from one nucleotide of the ITR closing base pair to the other nucleotide of the ITR closing base pair has a Gibbs free energy (ΔG) of unfolding under physiological conditions in the range of −26 kcal/mol to −95 kcal/mol. In some embodiments, the ITR sequence stretching from one nucleotide of the ITR closing base pair to the other nucleotide of the ITR closing base pair contribute to all of the Gibbs free energy (ΔG) of unfolding for the ITR sequence under physiological conditions.

In some embodiments, in the folded state, the single stranded IR or ITR has an overall Watson-Crick self-complementarity of approximately 50% to 98%. In one embodiment, in the folded state, the single stranded IR or ITR has an overall Watson-Crick self-complementarity of about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%. In another embodiment, in the folded state, the single stranded IR or ITR has an overall Watson-Crick self-complementarity of at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%. In some embodiments, in the folded state, IR or ITR has an overall Watson Crick complementarity of approximately 60% to 98%. In some embodiments, the single stranded IR or ITR has an overall GC content of approximately 60-95%. In certain embodiments, the single stranded IR or ITR has an overall GC content of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95%. In other embodiments, the single stranded IR or ITR has an overall GC content of about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, or about 95%. In some embodiments, the single stranded IR has an overall GC content of approximately 60-91%.

Table 4 lists the folding free energy, GC content, percent of complementation, length of exemplary ITRs and Table 5 lists the Sequences of the ITRs in Table 4.

TABLE 4
Folding free energy, GC content, percent of complementation, length of exemplary ITRs.

| | | | | | Paired | GC | ΔG | Compl. | Unpaired |
|---|---|---|---|---|---|---|---|---|---|
| ITR | Length | A-T | G-C | G-T | Total | % | kcal/mol | % | % |
| SEQ ID NO: 3 | 85 | 8 | 31 | | 39 | 79% | −83.0 | 92% | 8% |
| SEQ ID NO: 4 | 77 | 7 | 28 | | 35 | 80% | −72.7 | 91% | 9% |
| SEQ ID NO: 5 | 69 | 5 | 26 | | 31 | 84% | −63.6 | 90% | 10% |
| SEQ ID NO: 7 | 89 | 7 | 34 | | 41 | 83% | −90.0 | 92% | 8% |
| SEQ ID NO: 8 | 71 | 6 | 26 | | 32 | 81% | −65.2 | 90% | 10% |
| SEQ ID NO: 9 | 59 | 4 | 22 | | 26 | 85% | −50.7 | 88% | 12% |
| SEQ ID NO: 10 | 51 | 2 | 20 | | 22 | 91% | −41.9 | 86% | 14% |
| SEQ ID NO: 27 | 70 | 7 | 13 | | 20 | 65% | −26.6 | 57% | 43% |
| SEQ ID NO: 29 | 92 | 6 | 18 | 1 | 25 | 75% | −52.1 | 52% | 48% |
| SEQ ID NO: 28 | 102 | 12 | 26 | | 38 | 68% | −72.8 | 75% | 25% |
| SEQ ID NO: 31 | 87 | 13 | 23 | | 36 | 64% | −63.0 | 83% | 17% |
| SEQ ID NO: 32 | 113 | 18 | 31 | | 49 | 63% | −93.6 | 87% | 13% |
| SEQ ID NO: 33 | 83 | 6 | 32 | | 38 | 84% | −83.0 | 92% | 8% |
| SEQ ID NO: 34 | 83 | 7 | 31 | | 38 | 82% | −80.0 | 92% | 8% |
| SEQ ID NO: 35 | 67 | 6 | 26 | | 32 | 81% | −79.1 | 96% | 4% |

TABLE 5
Sequences of the ITRs in Table 4

| SEQ ID | |
|---|---|
| NO | Sequence |
| SEQ ID | GCTCGACTCGCTCACTGAGGCCGGG |
| NO: 3 | CGACCAAAGGTCGCCCGACGCCCGG |
| | GCTTTGCCCGGGCGGCCTCAGTGAG |
| | CGAGTCGAGC |
| SEQ ID | GCTCGACTCACTGAGGCCGGGCGAC |
| NO: 4 | CAAAGGTCGCCCGACGCCCGGGCTT |
| | TGCCCGGGCGGCCTCAGTGAGTCGA |
| | GC |
| SEQ ID | CGCTGACTCAGGCCGGGCGACCAAA |
| NO: 5 | GGTCGCCCGACGCCCGGGCTTTGCC |
| | CGGGCGGCCTGAGTCAGCG |
| SEQ ID | CGCGCTCGCTCGCTCACTGAGGCCG |
| NO: 7 | GGCGACCAAAGGTCGCCCGACGCCC |
| | GGGCTTTGCCCGGGCGGCCTCAGTG |
| | AGCGAGCGAGCGCG |
| SEQ ID | TCGCTCACTGAGGCCGGGCGACCAA |
| NO: 8 | AGGTCGCCCGACGCCCGGGCTTTGC |
| | CCGGGCGGCCTCAGTGAGCGA |
| SEQ ID | ACTGAGGCCGGGCGACCAAAGGTCG |
| NO: 9 | CCCGACGCCCGGGCTTTGCCCGGGC |
| | GGCCTCAGT |
| SEQ ID | AGGCCGGGCGACCAAAGGTCGCCCG |
| NO: 10 | ACGCCCGGGCTTTGCCCGGGCGGCC |
| | T |
| SEQ ID | CCATGCATCCGGCTTTAAACGGGCA |
| NO: 27 | ACTGCGTCTCATTCACGTTAGAGAC |
| | TACAACCGTCGGATGCATGG |
| SEQ ID | TTCAAACCTGCCGGGGGAGAAGCGG |
| NO: 28 | CGTTTTTTCCCGGCCGCCGCTTCTC |
| | TTCTTCTCCCGCCGCCGGGAAAAAA |
| | GGCGGGAGAAGCCCCGGCAGGTTTG |
| | AA |
| SEQ ID | GTCCGGGCCATGCTTCAAACCTGCC |
| NO: 29 | GGGGCTTCTCCCGCCTTTTTTCCCG |
| | GCGGCGGGAGAAGTAGATTTCTCGT |
| | ACCTGCATGGCCCGGAC |
| SEQ ID | CCAGCGCTTGGGGTTGACGTGCCAC |
| NO: 31 | TAAGATCAAGCGGCGCGCGCGCGCC |
| | GCTTGTCTTAGTGTCAAGGCAACCC |
| | CAAGCAAGCTGG |
| SEQ ID | GGTTGACTCTGGGCCAGCTTGCTTG |
| NO: 32 | GGGTTGCCTTGACACTAAGACAAGC |
| | GGCGCGCGCGCGCCGCTTGATCTTA |
| | GTGGCACGTCAACCCCAAGCGCTGG |
| | CCCAGAGTCAACC |
| SEQ ID | CGCGCTCGCTCGCTCACTGAGGCCG |
| NO: 33 | GGCCAAAGGCCCGACGCCCGGGCTT |
| | TGCCCGGGCGGCCTCAGTGAGCGAG |
| | CGAGCGCG |
| SEQ ID | CGCGCTCGCTCGCTCACTGAGGCCG |
| NO: 34 | GGCGACCAAAGGTCGCCCGACGCCC |
| | GTTTCGGGCGGCCTCAGTGAGCGAG |
| | CGAGCGCG |
| SEQ ID | CGCGCTCGCTCGCTCACTGAGGCCG |
| NO: 35 | CCCGGGCTTTGCCCGGGCGGCCTCA |
| | GTGAGCGAGCGAGCGCG |

The DNA molecules for the methods and compositions provided herein can comprise IR or ITRs of various origins. In one embodiment, the IR or ITR in the DNA molecule is a viral ITR. “Viral ITR” includes any viral terminal repeat or synthetic sequence that comprises at least one minimal required origin of replication and a region comprising a palindrome hairpin structure. In one embodiment, the viral ITR is derived from Parvoviridae. In another embodiment, the viral ITR derived from Parvoviridae comprises a minimal required origin of replication that comprises at least one viral replication-associated protein binding sequence (“RABS”), which refers to a DNA sequence to which viral DNA replication-associated proteins (“RAPs”) and isoforms thereof, encoded by the Parvoviridae genes Rep and/or NS1 can bind. In some embodiments, the RABS is a Rep binding sequence (“RBS”). In some embodiments the RABS comprises a Rep binding sequence (“RBS”), Rep can bind to two elements within the ITR. It can bind to a nucleotide sequence in the stem structure of the ITR (i.e., the nucleotide sequence recognized by a Rep protein for replication of viral nucleic acid molecules). Such an RBS is also referred to as RBE (Rep-binding element). Rep can also bind to a nucleotide sequence. which forms a small palindrome comprising a single tip of an internal hairpin within the ITR, thereby stabilizing the association between Rep and the ITR. Such an RBS is also referred to as RBE′. In another embodiment, the viral ITR derived from Parvoviridae comprises an RABS which comprises NS1-binding elements (“NSBEs”) that replication-associated viral protein NS1 can bind. In another embodiment, the RABS is an NS1-binding element (“NSBE”) to which replication-associated viral protein NS1 can bind. In some embodiments, viral ITR is derived from Parvoviridae and comprises a terminal resolution site (“TRS”) at which the viral DNA replication-associated proteins NS1 and/or Rep can perform an endonucleolytic nick within a sequence at the TRS. In yet another embodiment, the viral ITR comprises at least one RBS or NSBE and at least one TRS. In the context of a virus or recombinant RAP (i.e. Rep or NS1) based production of viral genomes, the ITRs mediate replication and virus packaging. As unexpectedly found and provided herein, duplex linear DNA vectors with ITRs similar to viral ITRs can be produced without the need for Rep or NS1 proteins and consequently independent of the RABS or TRS sequence for DNA replication. Accordingly, the RABS and TRS can optionally be encoded in the nucleotide sequence disclosed herein but are not required and offer flexibility with regard to designing the ITRs. In one embodiment, the ITR for the methods and compositions provided herein does not comprise at least one RABS (e.g., one RABS, two RABS, or more than two RABS). In another embodiment, the ITR for the methods and compositions provided herein does not comprise any RABS. In another embodiment, the ITR for the methods and compositions provided herein does not comprise at least one RBS. In another embodiment, the ITR for the methods and compositions provided herein does not comprise any RBS. In another embodiment, the ITR for the methods and compositions provided herein does not comprise RBE. In another embodiment, the ITR for the methods and compositions provided herein does not comprise RBE′. In another embodiment, the ITR for the methods and compositions provided herein does not comprise RBE and RBE′. In another embodiment, the ITR for the methods and compositions provided herein does not comprise NSBE. In yet another embodiment, the ITR for the methods and compositions provided herein does not comprise TRS. In a further embodiment, the ITR for the methods and compositions provided herein does not comprise at least one RABS (e.g., one RABS, two RABS, or more than two RABS) and does not comprise TRS. In a further embodiment, the ITR for the methods and compositions provided herein does not comprise any RABS and does not comprise TRS. In a further embodiment, the ITR for the methods and compositions provided herein comprises RBS (i.e., RBE and/or RBE′), TRS, or both RBS (i.e., RBE and/or RBE′) and TRS. In a further embodiment, the ITR for the methods and compositions provided herein comprises NBSE, TRS, or both NBSE and TRS.

“An ITR pair” refers to two ITRs within a single DNA molecule. In some embodiments, the two ITRs in the ITR pair are both derived from wild type viral ITRs (e.g. AAV2 ITR) that have an inverse complement sequence across their entire length. An ITR can be considered to be a wild-type sequence, even if it has one or more nucleotides that deviate from the canonical naturally occurring sequence, so long as the changes do not affect the properties and overall three-dimensional structure of the sequence. The disclosure provides that, in some embodiments, the insertion, deletion or substitution of one or more nucleotides can provide the generation of a restriction site for nicking endonuclease without changing the overall three-dimensional structure of the viral ITR. In some aspects, the deviating nucleotides represent conservative sequence changes. In certain embodiments, the sequence of an ITR provided herein can have at least 95%, at least 96%, at least 97%, at least 98%, or at least 99% sequence identity to the canonical sequence (as measured, e.g., using BLAST at default settings), and also has a restriction site for nicking endonuclease, such that the 3D structures are the same shape in geometrical space. In other embodiments, the sequence of an ITR provided herein can have about 95%, about 96%, about 97%, about 98%, or about 99% sequence identity to the canonical sequence (as measured, e.g., using BLAST at default settings), and also has a restriction site for nicking endonuclease, such that the 3D structures are the same shape in geometrical space.

In some embodiments, a DNA molecule for the methods and compositions provided herein comprises a pair of wt-ITRs. In certain specific embodiments, a DNA molecule for the methods and compositions provided herein comprises a pair of wt-ITRs selected from the group shown in Table 6. Table 6 shows exemplary ITRs from the same serotype or different serotypes, or different parvoviruses, including AAV serotype 1 (AAV1), AAV serotype 2 (AAV2), AAV serotype 3 (AAV3), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), or AAV serotype 12 (AAV12); AAVrh8, AAVrhlO, AAV-DJ, and AAV-DJ8 genome (e.g., NCBI: NC 002077; NC 001401; NC001729; NC001829; NC006152; NC 006260; NC 006261), ITRs from warm-blooded animals (avian AAV (AAAV), bovine AAV (BAAV), canine, equine, and ovine AAV), ITRs from B19 parvovirus (GenBank Accession No: NC 000883), Minute Virus from Mouse (MVM) (GenBank Accession No. NC 001510); Goose: goose parvovirus (GenBank Accession No. NC 001701); snake: snake parvovirus 1 (GenBank Accession No. NC 006148).

TABLE 6
Exemplary ITR sequences

| Virus | | |
|---|---|---|
| (accession | | |
| number) | Left ITR | Right ITR |
| AAV1 | TTGCCCACT | TTGCCCACT |
| | CCCTCTCTG | CCCTCTCTG |
| | CGCGCTCGC | CGCGCTCGC |
| | TCGCTCGGT | TCGCTCGGT |
| | GGGGCCTGC | GGGGCCTGC |
| | GGACCAAAG | GGACCAAAG |
| | GTCCGCAGA | GTCCGCAGA |
| | CGGCAGAGC | CGGCAGAGC |
| | TCTGCTCTG | TCTGCTCTG |
| | CCGGCCCCA | CCGGCCCCA |
| | CCGAGCGAG | CCGAGCGAG |
| | CGAGCGCGC | CGAGCGCGC |
| | AGAGAGGGA | AGAGAGGGA |
| | GTGGGCAA | GTGGGCAA |
| | (SEQ ID | (SEQ ID |
| | NO: 11) | NO: 12) |
| AAV2 | TTGGCCACT | TTGGCCACT |
| | CCCTCTCTG | CCCTCTCTG |
| | CGCGCTCGC | CGCGCTCGC |
| | TCGCTCACT | TCGCTCACT |
| | GAGGCCGGG | GAGGCCGGG |
| | CGACCAAAG | CGACCAAAG |
| | GTCGCCCGA | GTCGCCCGA |
| | CGCCCGGGC | CGCCCGGGC |
| | TTTGCCCGG | TTTGCCCGG |
| | GCGGCCTCA | GCGGCCTCA |
| | GTGAGCGAG | GTGAGCGAG |
| | CGAGCGCGC | CGAGCGCGC |
| | AGAGAGGGA | AGAGAGGGA |
| | GTGGCCAA | GTGGCCAA |
| | (SEQ ID | (SEQ ID |
| | NO: 13) | NO: 14) |
| AAV3 | TTGGCCACT | TTGGCCACT |
| | CCCTCTATG | CCCTCTATG |
| | CGCACTCGC | CGCACTCGC |
| | TCGCTCGGT | TCGCTCGGT |
| | GGGGCCTGG | GGGGCCTGG |
| | CGACCAAAG | CGACCAAAG |
| | GTCGCCAGA | GTCGCCAGA |
| | CGGACGTGC | CGGACGTGC |
| | TTTGCACGT | TTTGCACGT |
| | CCGGCCCCA | CCGGCCCCA |
| | CCGAGCGAG | CCGAGCGAG |
| | CGAGTGCGC | CGAGTGCGC |
| | ATAGAGGGA | ATAGAGGGA |
| | GTGGCCAA | GTGGCCAA |
| | (SEQ ID | (SEQ ID |
| | NO: 15) | NO: 16) |
| AAV4 | TTGGCCACT | CTATGCGCG |
| | CCCTCTATG | CTCGCTCAC |
| | CGCGCTCGC | TCACTCGGC |
| | TCACTCACT | CCTGGAGAC |
| | CGGCCCTGG | CAAAGGTCT |
| | AGACCAAAG | CCAGACTGC |
| | GTCTCCAGA | CGGCCTCTG |
| | CTGCCGGCC | GCCGGCAGG |
| | TCTGGCCGG | GCCGAGTGA |
| | CAGGGCCGA | GTGAGCGAG |
| | GTGAGTGAG | CGCGCATAG |
| | CGAGCGCGC | AGGGAGTGG |
| | ATAGAGGGA | CCAA |
| | GTGGCCAA | (SEQ ID |
| | (SEQ ID | NO: 18) |
| | NO: 17) | |
| AAV5 | CTCTCCCCC | CTCTCCCCC |
| (NC_0061 | CTGTCGCGT | CTGTCGCGT |
| 52) | TCGCTCGCT | TCGCTCGCT |
| | CGCTGGCTC | CGCTGGCTC |
| | GTTTGGGGG | GTTTGGGGG |
| | GGTGGCAGC | GGTGGCAGC |
| | TCAAAGAGC | TCAAAGAGC |
| | TGCCAGACG | TGCCAGACG |
| | ACGGCCCTC | ACGGCCCTC |
| | TGGCCGTCG | TGGCCGTCG |
| | CCCCCCCAA | CCCCCCCAA |
| | ACGAGCCAG | ACGAGCCAG |
| | CGAGCGAGC | CGAGCGAGC |
| | GAACGCGAC | GAACGCGAC |
| | AGGGGGGAG | AGGGGGGAG |
| | AG | AG |
| | (SEQ ID | (SEQ ID |
| | NO: 19) | NO: 20) |
| AAV7 | TTGGCCACT | TTGGCCACT |
| (NC_0062 | CCCTCTATG | CCCTCTATG |
| 60) | CGCGCTCGC | CGCGCTCGC |
| | TCGCTCGGT | TCGCTCGGT |
| | GGGGCCTGC | GGGGCCTGC |
| | GGACCAAAG | GGACCAAAG |
| | GTCCGCAGA | GTCCGCAGA |
| | CGGCAGAGC | CGGCAGAGC |
| | TCTGCTCTG | TCTGCTCTG |
| | CCGGCCCCA | CCGGCCCCA |
| | CCGAGCGAG | CCGAGCGAG |
| | CGAGCGCGC | CGAGCGCGC |
| | ATAGAGGGA | ATAGAGGGA |
| | GTGGCCAA | GTGGCCAA |
| | (SEQ ID | (SEQ ID |
| | NO: 21) | NO: 22) |
| HBOV | GTGGTTGTA | TTGCTTATG |
| (JQ92342 | CAGACGCCA | CAATCGCGA |
| 2) | TCTTGGAAT | AACTCTATA |
| | CCAATATGT | TCTTTTAAT |
| | CTGCCGGCT | GTGTTGTTG |
| | CAGTCATGC | TTGTACATG |
| | CTGCGCTGC | CGCCATCTT |
| | GCGCAGCGC | AGTTTTATA |
| | GCTGCGCGC | TCAGCTGGC |
| | GCGCATGAT | GCCTTAGTT |
| | CTAATCGCC | ATATAACAT |
| | GGCAGACAT | GCATGTTAT |
| | ATTGGATTC | ATAACTAAG |
| | CAAGATGGC | GCGCCAGCT |
| | GTCTGTACA | GATATAAAA |
| | ACCAC | CTAAGATGG |
| | (SEQ ID | CGCATGTAC |
| | NO: 23) | AACAACAAC |
| | | ACATTAAAA |
| | | GATATAGAG |
| | | TTTCGCGAT |
| | | TGCATAAGC |
| | | AA |
| | | (SEQ ID |
| | | NO: 24) |
| hB19 | TGGGCCAGC | TGGGCCAGC |
| (AY38633 | TTGCTTGGG | GCTTGGGGT |
| 0) | GTTGCCTTG | TGACGTGCC |
| | ACACTAAGA | ACTAAGATC |
| | CAAGCGGCG | AAGCGGCGC |
| | CGCCGCTTG | GCCGCTTGT |
| | ATCTTAGTG | CTTAGTGTC |
| | GCACGTCAA | AAGGCAACC |
| | CCCCAAGCG | CCAAGCAAG |
| | CTGGCCCA | CTGGCCCA |
| | (SEQ ID | (SEQ ID |
| | NO: 25) | NO: 26) |

In some embodiments, the DNA molecules for the methods and compositions provided herein comprise whole or part of the parvoviral genome. The parvoviral genome is linear, 3.9-6.3 kb in size, and the coding region is bracketed by terminal repeats that can fold into hairpin-like structures, which are either different (heterotelomeric, e.g. HBoV) or identical (homotelomeric, e.g. AAV2). In one embodiment, a DNA molecule for the methods and compositions provided herein comprises 2 different ITRs at the 2 ends of the DNA molecule. In another embodiment, a DNA molecule for the methods and compositions provided herein comprises 2 identical ITRs at the 2 ends of the DNA molecule. In yet another embodiment, a DNA molecule for the methods and compositions provided herein comprises 2 different ITRs at the 2 ends of the DNA molecule corresponding to the 2 HBoV ITRs. In a further embodiment, a DNA molecule for the methods and compositions provided herein comprises 2 identical ITRs at the 2 ends of the DNA molecule corresponding to the AAV2 ITR.

In certain embodiments, the ITR in the DNA molecules provided herein can be an AAV ITR. In other embodiments, the ITR can be a non-AAV ITR. In one embodiment, the ITRs in the DNA molecules provided herein can be derived from an AAV ITR or a non-AAV TR. In some specific embodiments, the ITR can be derived from any one of the family Parvoviridae, which encompasses parvoviruses and dependoviruses (e.g., canine parvovirus, bovine parvovirus, mouse parvovirus, porcine parvovirus, human parvovirus B-19). In other specific embodiments, the ITR can be derived from the SV40 hairpin that serves as the origin of SV40 replication. Parvoviridae family viruses consist of two subfamilies: Parvovirinae, which infect vertebrates, and Densovirinae, which infect invertebrates. As such, in one embodiment, the ITR can be derived from any one of the subfamily Parvovirinae. In another embodiment, the ITR can be derived from any one of the subfamily Densovirinae.

In comparison to the T-shaped AAV ITRs, the human erythrovirus B19 has ITRs that terminate in imperfect, palindromes that can fold into long linear duplexes with a few unpaired nucleotides, creating a series of small, but highly conserved, mismatched bulges. In some embodiments, any parvovirus ITR can be used as an ITR for the DNA molecules provided herein (e.g. wild type or modified ITR) or can act as a template ITR for modification and then incorporation in the DNA molecules provided herein. In some specific embodiments, the parvovirus, from which the ITRs of the DNA molecules are derived, is a dependovirus, an erythroparvovirus, or a bocaparvovirus. In other specific embodiments, the ITRs of the DNA molecules provided herein are derived from AAV, B19 or HBOV. In one specific embodiment, the ITRs of the DNA molecules provided herein are derived from HBOV genome (accession number JQ923422) nucleotides 19-122 (TCTTGGAATCCAATATGTCTGCCGGCTCAGTCATGCCTGCGCTGCGCGCAGCGC GCTGCGCGCGCGCATGATCTAATCGCCGGCAGACATATTGGATTCCAAGA SEQ ID NO. 183). In another specific embodiment, the ITRs of the DNA molecules provided herein are derived from B19 genome (accession number AY386330) nucleotides 129 to 237 (GGGTTGGCTCTGGGCCAGCTTGCTTGGGGTTGCCTTGACACTAAGACAAGCGGC GCGCCGCTTGATCTTAGTGGCACGTCAACCCCAAGCGCTGGCCCAGAGCCAACC C SEQ ID NO. 184) In certain embodiments, the serotype of AAV ITRs chosen for the DNA molecules provided herein can be based upon the tissue tropism of the serotype. AAV2 has a broad tissue tropism, AAV1 preferentially targets to neuronal and skeletal muscle, and AAV5 preferentially targets neuronal, retinal pigmented epithelia, and photoreceptors. AAV6 preferentially targets skeletal muscle and lung. AAV8 preferentially targets liver, skeletal muscle, heart, and pancreatic tissues. AAV9 preferentially targets liver, skeletal and lung tissue. In one embodiment, the ITR or modified ITR of the DNA molecules provided herein is based on an AAV2 ITR. In one embodiment, the ITR or modified ITR of the DNA molecules provided herein is based on an AAV1 ITR. In one embodiment, the ITR or modified ITR of the DNA molecules provided herein is based on an AAV5 ITR. In one embodiment, the ITR or modified ITR of the DNA molecules provided herein is based on an AAV6 ITR. In one embodiment, the ITR or modified ITR of the DNA molecules provided herein is based on an AAV8 ITR. In one embodiment, the ITR or modified ITR of the DNA molecules provided herein is based on an AAV9 ITR.

In one embodiment, the DNA molecules for the methods and compositions provided herein comprise one or more non-AAV ITR. In a further embodiment, such non-AAV ITR can be derived from hairpin sequences found in the mammalian genome. In one specific embodiment, such non-AAV ITR can be derived from the hairpin sequences found in the mitochondrial genome including the OriL hairpin sequence (SEQ ID NO:30: 5′ GAAGAGGGCGGCGGCCCTTTTTTCCGCCCTCTTCGGGGCCGTCCAAACTT′3), which adopts a stem-loop structure and is involved in initiating the DNA synthesis of mitochondrial DNA (see Fuste et al., Molecular Cell, 37, 67-78, Jan. 15, 2010, which is incorporated herein in its entirety by reference). In another specific embodiment, the DNA molecules for the methods and compositions provided herein comprise an ITR derived from the OriL sequence that is mirrored to form a T junction with two self-complimentary palindromic regions and a 12-nucleotide loop at either apex of the hairpin. In one embodiment the DNA molecules for the methods and compositions provided herein comprise an ITR derived from the OriL sequence that maintains OriL hairpin loop followed by an unpaired bulge and a GC-rich stem. Some exemplary embodiments of the ITRs derived from mitochondria OriL are depicted in FIG. 4.

In one embodiment, the DNA molecules for the methods and compositions provided herein comprise one or more non-AAV ITRs that are derived from aptamer. Similar to viral ITRs, aptamers are composed of ssDNA that folds into a three-dimensional structure and have the ability to recognize biological targets with high affinity and specificity. DNA aptamers can be generated by systematic evolution of ligands by exponential enrichment (SELEX). For example, it has previously been shown that some aptamers can target the nuclei of human cells (See Shen et al ACS Sens. 2019, 4, 6, 1612-1618, which is herein incorporated in its entirety by reference). In one embodiment, the DNA molecules for the methods and compositions provided herein comprise nucleus targeting aptamer ITRs or their derivatives, wherein the aptamer specifically binds nuclear protein. In some embodiments, the aptamer ITRs fold into a secondary structure that can contain such as hairpins as well as internal loops as well bulges and a stem region. Some exemplary embodiments of aptamers or the ITRs derived from are depicted in FIG. 5. In specific embodiments, the aptamer comprises the sequence

(SEQ ID NO: 346)
ATCCGGCTTTAAACGGGCAACTGCGTCTCATT
CACGTTAGAGACTACAACCGTCGGAT

In some specific embodiments, the DNA molecules for the methods and compositions provided herein comprise one or more AAV2 ITR, human erythrovirus B19 ITR goose parvovirus ITR, and/or their derivatives in any combination. In other specific embodiments, the DNA molecules for the methods and compositions provided herein comprise two ITRs selected from AAV2 ITR, human erythrovirus B19 ITR goose parvovirus ITR, and their derivatives, in any combination. In some specific embodiments, the DNA molecules for the methods and compositions provided herein comprise one or more AAV2 ITR, human erythrovirus B19 ITR goose parvovirus ITR, and/or their derivatives, in any combination, wherein the ITRs remain functional regardless of whether the palindromic regions of their ITRs are in direct, reverse, or any possible combination of 5′ and 3′ ITR directionality with respect to the expression cassette (as described in WO2019143885, which is herein incorporated in its entirety by reference).

In some embodiments, a modified IR or ITR in the DNA molecules provided herein is a synthetic IR sequence that comprises a restriction site for endonuclease such as 5′-GAGTC-3′ in addition to various palindromic sequence allowing for hairpin secondary structure formation as described in this Section (Section 5.4.1).

In certain embodiments, the IR or ITR in the DNA molecules provided herein can be an IR or ITR having various sequence homology with the IR or ITR sequences described in this Section (Section 5.4.1). In other embodiments, the IR or ITR in the DNA molecules provided herein can be an IR or ITR having various sequence homology with the known IR or ITR sequences of various ITR origins described in this Section (Section 5.4.1) (e.g. viral ITR, mitochondria ITR, artificial or synthetic ITR such as aptamers, etc.). In one embodiment, such homology provided in this paragraph can be a homology of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%. In another embodiment, such homology provided in this paragraph can be a homology of about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%.

In certain embodiments, the right and/or left IRs or ITRs are synthetic IRs or ITRs. In certain embodiments, the synthetic IR or ITR comprises the nucleotide sequence of SEQ ID NO: 417 or 418. In certain embodiments, the synthetic IR or ITR comprises a nucleotide sequence which is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical to SEQ ID NO: 417 or 418.

In some embodiments, the IR or ITR in the DNA molecules provided herein can comprise any one or more features described in this Section (Section 5.4.1), in various permutations and combinations.

5.4.2 Restriction Enzymes, Nicking Endonucleases, and their Respective Restriction Sites; Programmable Nicking Enzymes and their Targeting Sites

Various embodiments for the nicking endonucleases, restriction enzymes, and/or their respective restriction sites as describe in Section 5.3.4 are provided for the DNA molecules provided herein. In some embodiments, the first, second, third, and fourth restriction sites for nicking endonuclease provided for the DNA molecules as described in Section 3 and this Section (Section 5.4) can be all target sequences for the same nicking endonuclease. In some embodiments, the first, second, third, and fourth restriction sites for nicking endonuclease provided for the DNA molecules as described in Section 3 and this Section (Section 5.4) can be target sequences for four different nicking endonucleases. In other embodiments, the first, second, third, and fourth restriction sites for nicking endonucleases are target sequences for two different nicking endonucleases, including all possible combinations of arranging the four sites for two different nicking endonuclease target sequences (e.g. the first restriction site for the first nicking endonuclease and the rest for the second nicking endonuclease, the first and second restriction sites for the first nicking endonuclease and the rest for the second nicking endonuclease, etc.). In certain embodiments, the first, second, third, and fourth restriction sites for nicking endonucleases are target sequences for three different nicking endonucleases, including all possible combinations of arranging the four sites for three different nicking endonuclease target sequences. In some embodiments, the nicking endonuclease and restriction sites for the nicking endonuclease can be any one selected from those described in Section 5.3.4, including Table 2. In further embodiments, each of the first, second, third, and fourth restriction site for nicking endonuclease can be a site for any nicking endonuclease selected from those described in Section 5.3.4, including Table 2.

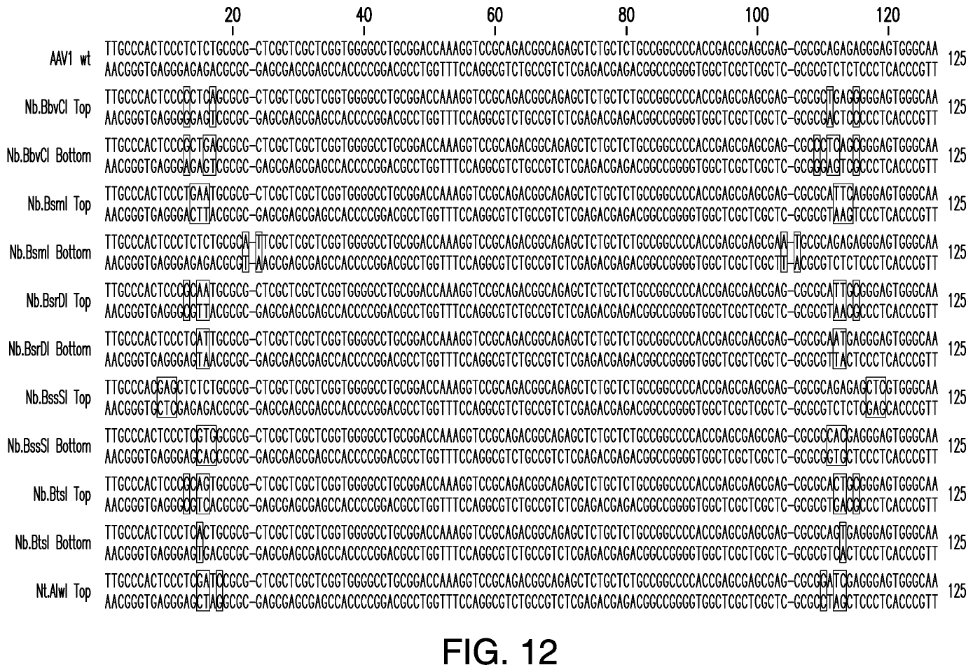

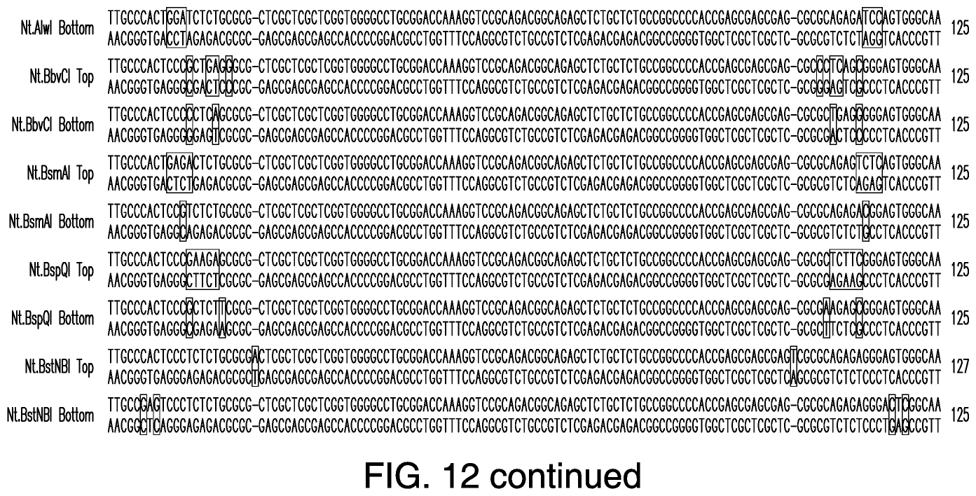

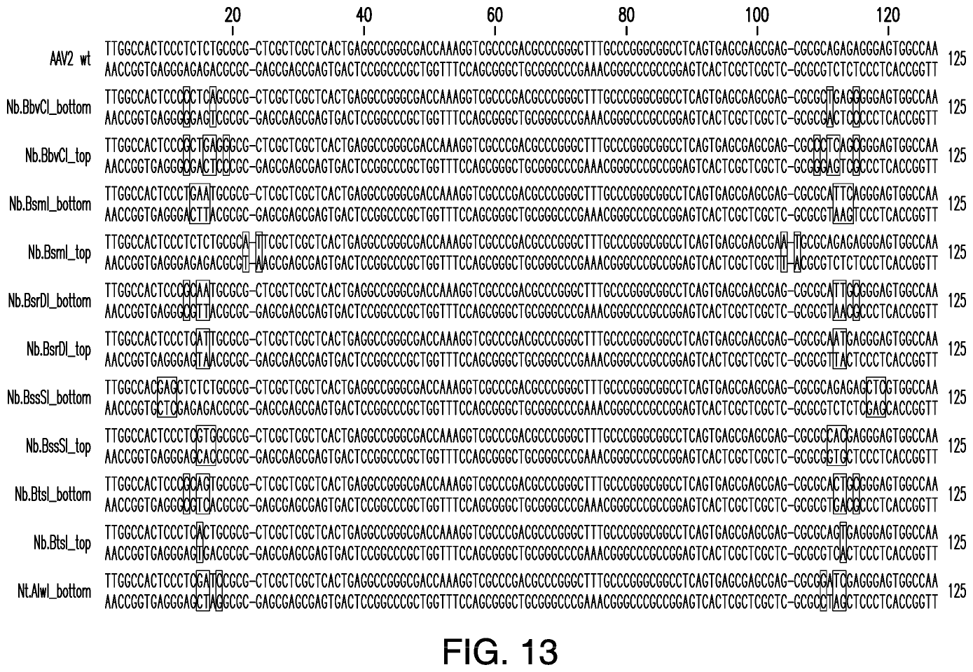

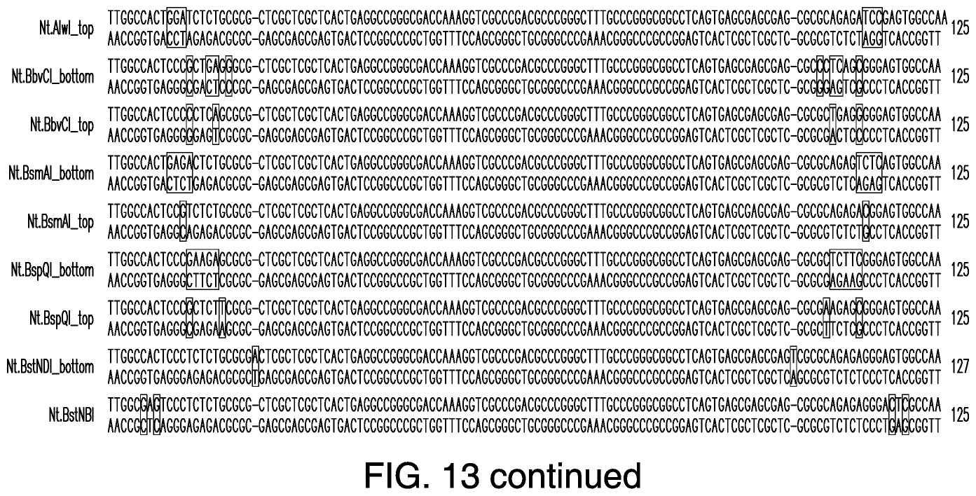

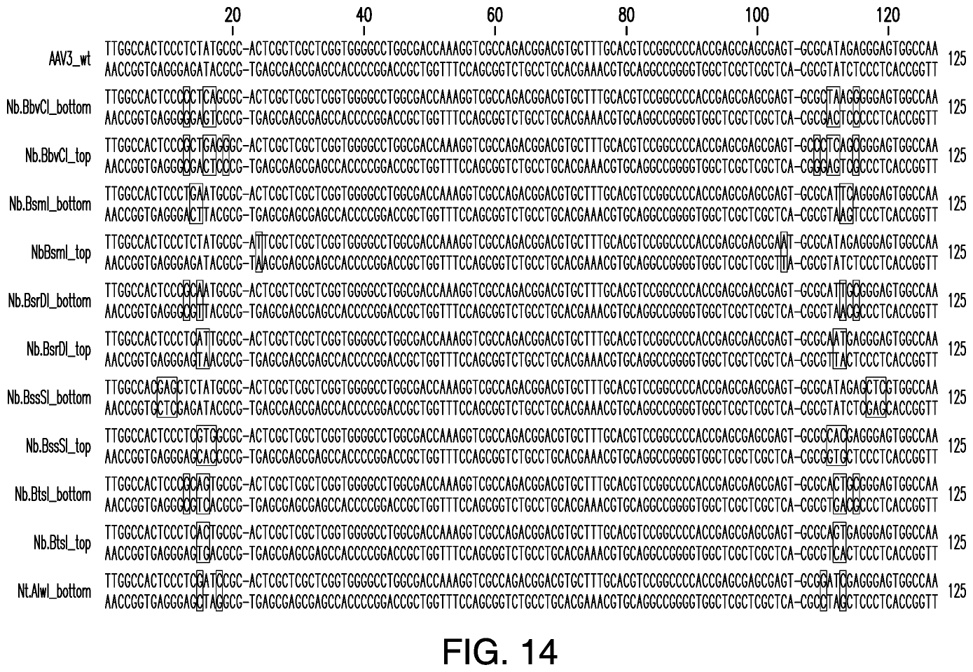

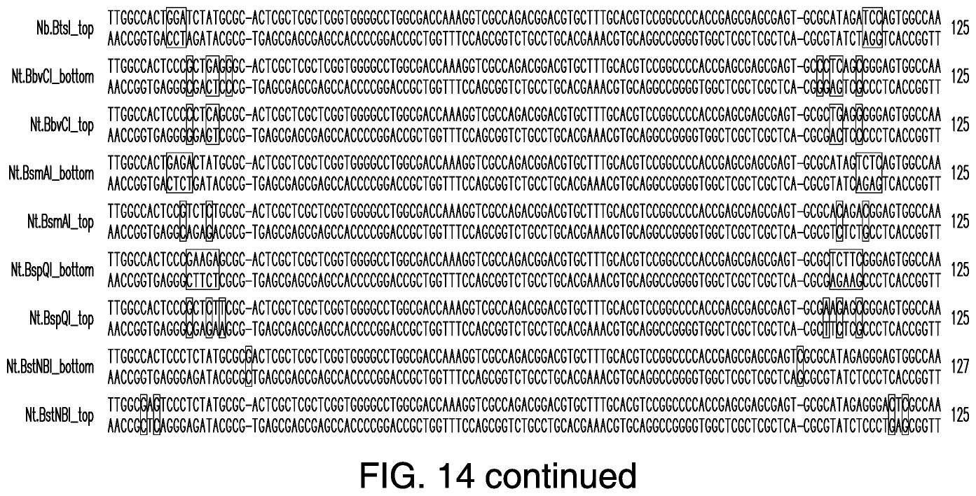

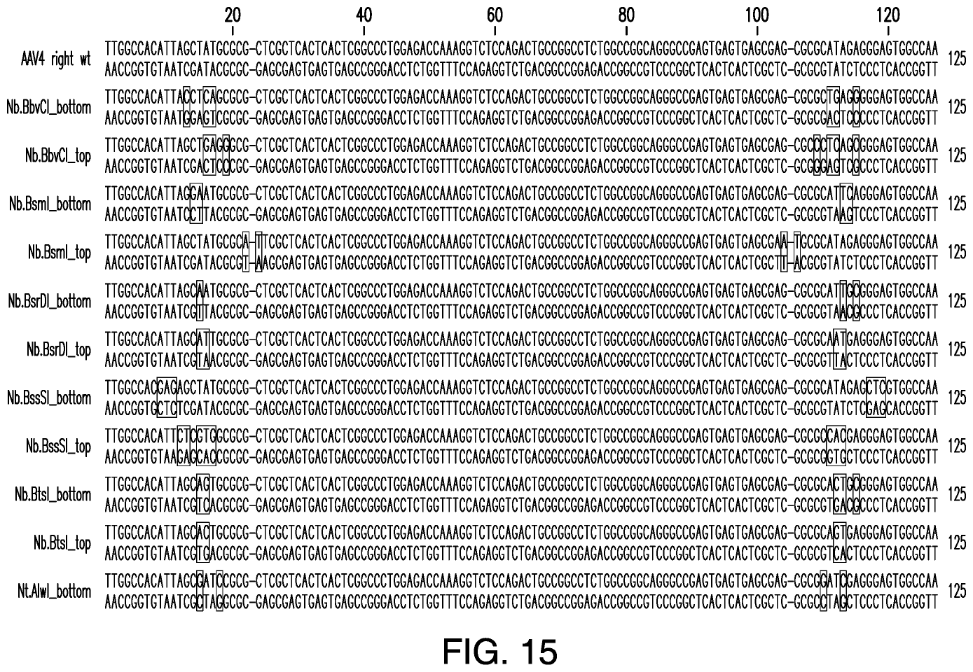

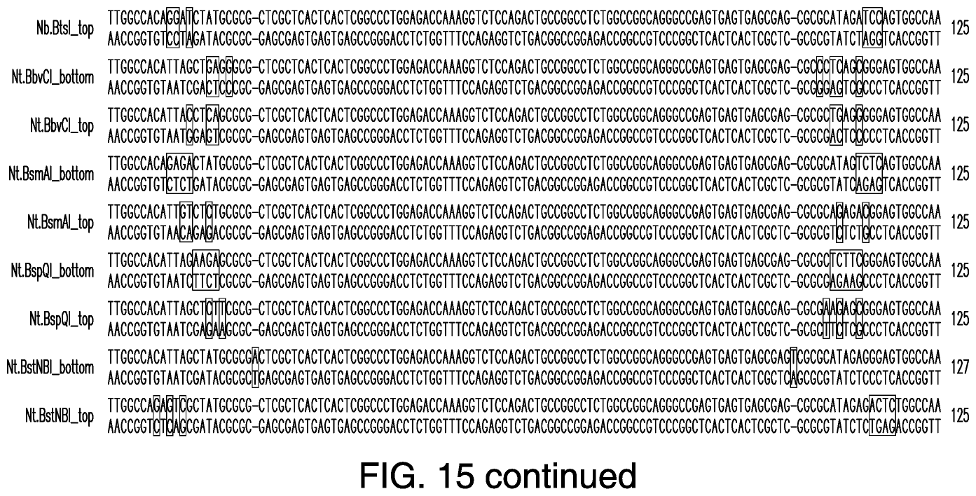

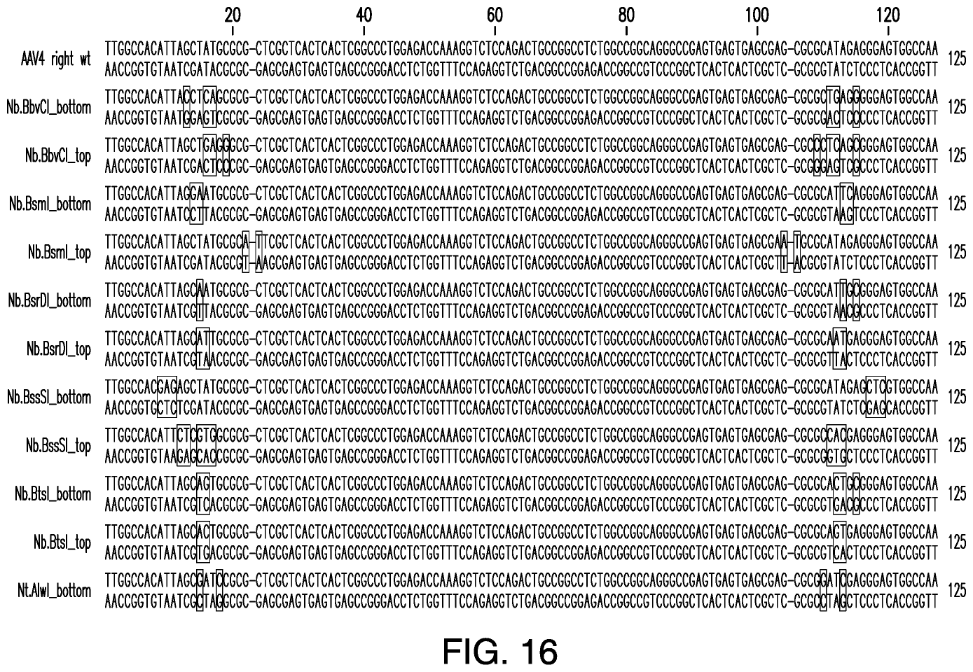

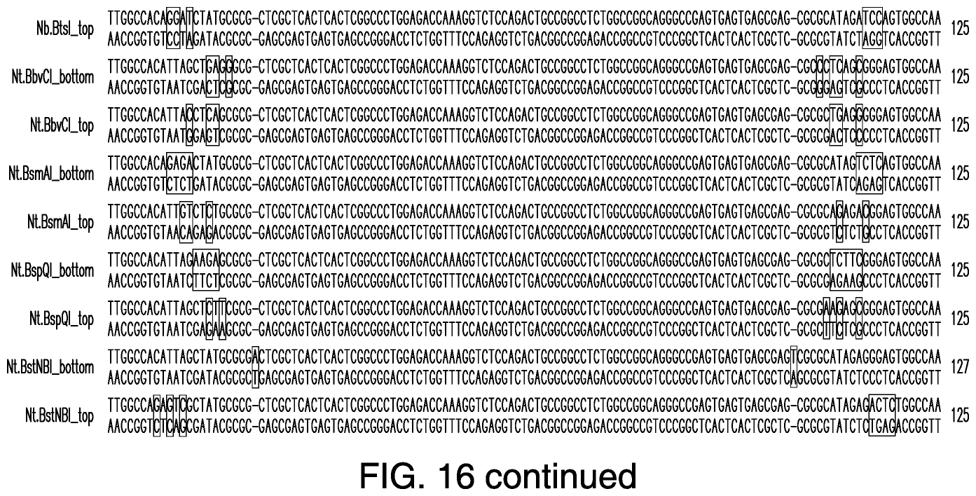

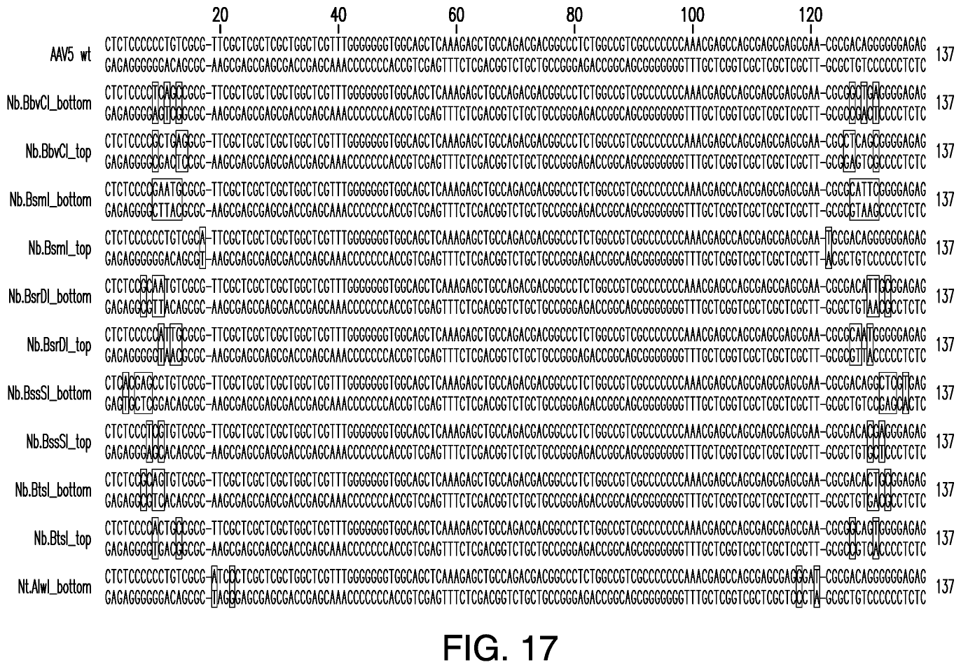

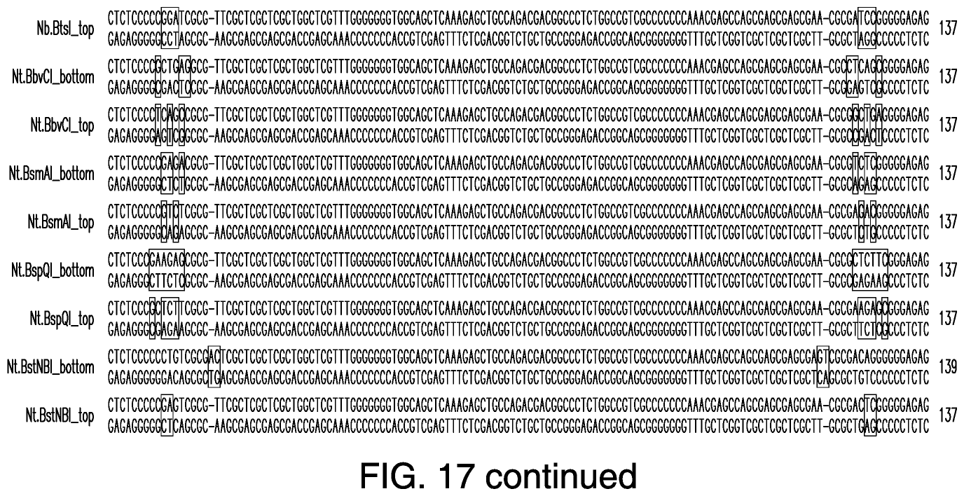

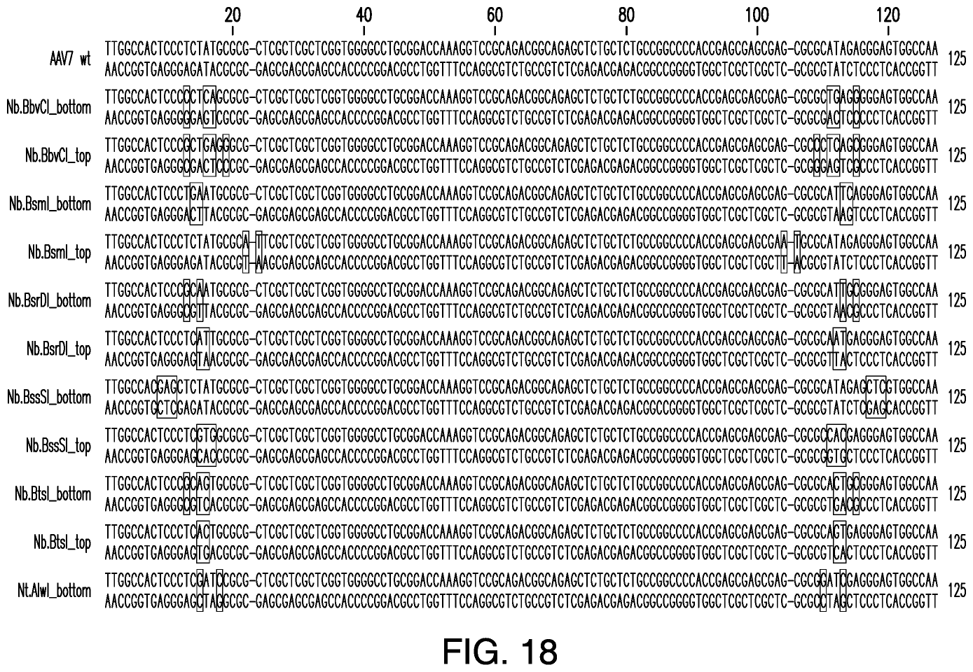

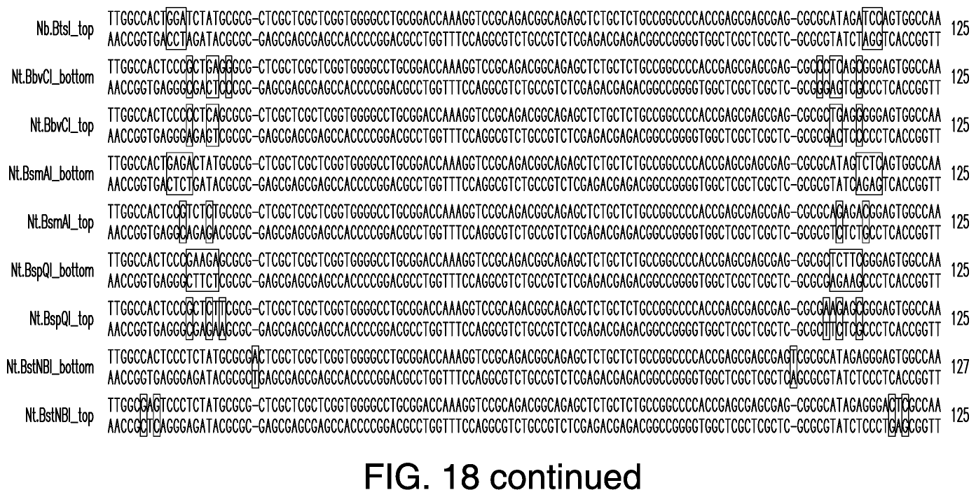

Table 7 to Table 16 show exemplary modified AAV ITR sequences that harbor two antiparallel recognition sites for the same nicking endonuclease, grouped by nicking endonuclease species. The corresponding alignments for modified sequences of ITRs and wild type of AAV1, AAV2, AAV3, AAV4 left, AAV4 Right, AAV5 and AAV7 are depicted in FIG. 12 to FIG. 18.

TABLE 7
Exemplary AAV derived ITRs harboring
antiparallel recognition sites for
nicking endonuclease Nb.BvCI:

| SEQ | | |
|---|---|---|
| ID | | |
| No: | Name | Full Sequence |
| SEQ | source: | TTGCCCACTCCCCCTCA |
| ID | AAV1; | GCGCGCTCGCTCGCTCG |
| No: | Recogn. | GTGGGGCCTGCGGACCA |
| 1 | Site: | AAGGTCCGCAGACGGCA |
| | Nb.BbvCI; | GAGCTCTGCTCTGCCGG |
| | Format: | CCCCACCGAGCGAGCGA |
| | b1 | GCAGCGCTGAGGGGGAG |
| | | TGGGCA |
| SEQ | source: | TTGCCCACTCCCGCTGA |
| ID | AAV1; | GGGCGCTCGCTCGCTCG |
| No: | Recogn. | GTGGGGCCTGCGGACCA |
| 2 | Site: | AAGGTCCGCAGACGGCA |
| | Nb.BbvCI; | GAGCTCTGCTCTGCCGG |
| | Format: | CCCCACCGAGCGAGCGA |
| | t1 | GCGCCCTCAGCGGGAGT |
| | | GGGCAA |
| SEQ | source: | TTGGCCACTCCCCCTCA |
| ID | AAV2; | GCGCGCTCGCTCGCTCA |
| No: | Recogn. | CTGAGGCCGGGCGACCA |
| 36 | Site: | AAGGTCGCCCGACGCCC |
| | Nb.BbvCI; | GGGCTTTGCCCGGGCGG |
| | Format: | CCTCAGTGAGCGAGCGA |
| | b1 | GCGCGCTGAGGGGGAGT |
| | | GGCCAA |
| SEQ | source: | TTGGCCACTCCCGCTGA |
| ID | AAV2; | GGGCGCTCGCTCGCTCA |
| No: | Recogn. | CTGAGGCCGGGCGACCA |
| 37 | Site: | AAGGTCGCCCGACGCCC |
| | Nb.BbvCI; | GGGCTTTGCCCGGGCGG |
| | Format: | CCTCAGTGAGCGAGCGA |
| | t1 | GCGCCCTCAGCGGGAGT |
| | | GGCCAA |
| SEQ | source: | TTGGCCACTCCCCCTCA |
| ID | AAV3; | GCGCACTCGCTCGCTCG |
| No: | Recogn. | GTGGGGCCTGGCGACCA |
| 38 | Site: | AAGGTCGCCAGACGGAC |
| | Nb.BbvCI; | GTGCTTTGCACGTCCGG |
| | Format: | CCCCACCGAGCGAGCGA |
| | b1 | GTGCGCTGAGGGGGAGT |
| | | GGCCAA |
| SEQ | source: | TTGGCCACTCCCGCTGA |
| ID | AAV3; | GGGCACTCGCTCGCTCG |
| No: | Recogn. | GTGGGGCCTGGCGACCA |
| 39 | Site: | AAGGTCGCCAGACGGAC |
| | Nb.BbvCI; | GTGCTTTGCACGTCCGG |
| | Format: | CCCCACCGAGCGAGCGA |
| | t1 | GTGCCCTCAGCGGGAGT |
| | | GGCCAA |
| SEQ | source: | TTGGCCACTCCCCCTCA |
| ID | AAV4 | GCGCGCTCGCTCACTCA |
| No: | left; | CTCGGCCCTGGAGACCA |
| 40 | Recogn. | AAGGTCTCCAGACTGCC |
| | Site: | GGCCTCTGGCCGGCAGG |
| | Nb.BbvCI; | GCCGAGTGAGTGAGCGA |
| | Format: | GCGCGCTGAGGGGGAGT |
| | b1 | GGCCAA |
| SEQ | source: | TTGGCCACTCCCGCTGA |
| ID | AAV4 | GGGCGCTCGCTCACTCA |
| No: | left; | CTCGGCCCTGGAGACCA |
| 41 | Recogn. | AAGGTCTCCAGACTGCC |
| | Site: | GGCCTCTGGCCGGCAGG |
| | Nb.BbvCI; | GCCGAGTGAGTGAGCGA |
| | Format: | GCGCCCTCAGCGGGAGT |
| | t1 | GGCCAA |
| SEQ | source: | TTGGCCACATTACCTCA |
| ID | AAV4 | GCGCGCTCGCTCACTCA |
| No: | right; | CTCGGCCCTGGAGACCA |
| 42 | Recogn. | AAGGTCTCCAGACTGCC |
| | Site: | GGCCTCTGGCCGGCAGG |
| | Nb.BbvCI; | GCCGAGTGAGTGAGCGA |
| | Format: | GCGCGCTGAGGGGGAGT |
| | b1 | GGCCAA |
| SEQ | source: | TTGGCCACATTAGCTGA |
| ID | AAV4 | GGGCGCTCGCTCACTCA |
| No: | right; | CTCGGCCCTGGAGACCA |
| 43 | Recogn. | AAGGTCTCCAGACTGCC |
| | Site: | GGCCTCTGGCCGGCAGG |
| | Nb.BbvCI; | GCCGAGTGAGTGAGCGA |
| | Format: | GCGCCCTCAGCGGGAGT |
| | t1 | GGCCAA |
| SEQ | source: | CTCTCCCCTCAGCCGCG |
| ID | AAV5; | TTCGCTCGCTCGCTGGC |
| No: | Recogn. | TCGTTTGGGGGGGTGGC |
| 44 | Site: | AGCTCAAAGAGCTGCCA |
| | Nb.BbvCI; | GACGACGGCCCTCTGGC |
| | Format: | CGTCGCCCCCCCAAACG |
| | b1 | AGCCAGCGAGCGAGCGA |
| | | ACGCGGCTGAGGGGAGA |
| | | G |
| SEQ | source: | CTCTCCCCGCTGAGGCG |
| ID | AAV5; | TTCGCTCGCTCGCTGGC |
| No: | Recogn. | TCGTTTGGGGGGGTGGC |
| 45 | Site: | AGCTCAAAGAGCTGCCA |
| | Nb.BbvCI; | GACGACGGCCCTCTGGC |
| | Format: | CGTCGCCCCCCCAAACG |
| | t1 | AGCCAGCGAGCGAGCGA |
| | | ACGCCTCAGCGGGGAGA |
| | | G |
| SEQ | source: | TTGGCCACTCCCCCTCA |
| ID | AAV7; | GCGCGCTCGCTCGCTCG |
| No: | Recogn. | GTGGGGCCTGCGGACCA |
| 46 | Site: | AAGGTCCGCAGACGGCA |
| | Nb.BbvCI; | GAGCTCTGCTCTGCCGG |
| | Format: | CCCCACCGAGCGAGCGA |
| | b1 | GCGCGCTGAGGGGGAGT |
| | | GGCCAA |
| SEQ | source: | TTGGCCACTCCCGCTGA |
| ID | AAV7; | GGGCGCTCGCTCGCTCG |
| No: | Recogn. | GTGGGGCCTGCGGACCA |
| 47 | Site: | AAGGTCCGCAGACGGCA |
| | Nb.BbvCI; | GAGCTCTGCTCTGCCGG |
| | Format: | CCCCACCGAGCGAGCGA |
| | t1 | GCGCCCTCAGCGGGAGT |
| | | GGCCAA |

TABLE 8
Exemplary AAV derived ITRs harboring
antiparallel recognition sites for
nicking endonuclease Nb.BsmI:

| SEQ | | |
|---|---|---|
| ID | | |
| No: | Name | Full Sequence |
| SEQ | source: | TTGCCCACTCCCTGAAT |
| ID | AAV1; | GCGCGCTCGCTCGCTCG |
| No: | Recogn. | GTGGGGCCTGCGGACCA |
| 48 | Site: | AAGGTCCGCAGACGGCA |
| | Nb.BsmI; | GAGCTCTGCTCTGCCGG |
| | Format: | CCCCACCGAGCGAGCGA |
| | b1 | GCGCGCATTCAGGGAGT |
| | | GGGCAA |
| SEQ | source: | TTGCCCACTCCCTCTCT |
| ID | AAV1; | GCGCATTCGCTCGCTCG |
| No: | Recogn. | GTGGGGCCTGCGGACCA |
| 49 | Site: | AAGGTCCGCAGACGGCA |
| | Nb.BsmI; | GAGCTCTGCTCTGCCGG |
| | Format: | CCCCACCGAGCGAGCGA |
| | t1 | ATGCGCAGAGAGGGAGT |
| | | GGGCAA |
| SEQ | source: | TTGGCCACTCCCTGAAT |
| ID | AAV2; | GCGCGCTCGCTCGCTCA |
| No: | Recogn. | CTGAGGCCGGGCGACCA |
| 50 | Site: | AAGGTCGCCCGACGCCC |
| | Nb.BsmI; | GGGCTTTGCCCGGGCGG |
| | Format: | CCTCAGTGAGCGAGCGA |
| | b1 | GCGCGCATTCAGGGAGT |
| | | GGCCAA |
| SEQ | source: | TTGGCCACTCCCTCTCT |
| ID | AAV2; | GCGCATTCGCTCGCTCA |
| No: | Recogn. | CTGAGGCCGGGCGACCA |
| 51 | Site: | AAGGTCGCCCGACGCCC |
| | Nb.BsmI; | GGGCTTTGCCCGGGCGG |
| | Format: | CCTCAGTGAGCGAGCGA |
| | t1 | ATGCGCAGAGAGGGAGT |
| | | GGCCAA |
| SEQ | source: | TTGGCCACTCCCTGAAT |
| ID | AAV3; | GCGCACTCGCTCGCTCG |
| No: | Recogn. | GTGGGGCCTGGCGACCA |
| 52 | Site: | AAGGTCGCCAGACGGAC |
| | Nb.BsmI; | GTGCTTTGCACGTCCGG |
| | Format: | CCCCACCGAGCGAGCGA |
| | b1 | GTGCGCATTCAGGGAGT |
| | | GGCCAA |
| SEQ | source: | TTGGCCACTCCCTCTAT |
| ID | AAV3; | GCGCATTCGCTCGCTCG |
| No: | Recogn. | GTGGGGCCTGGCGACCA |
| 53 | Site: | AAGGTCGCCAGACGGAC |
| | Nb.BsmI; | GTGCTTTGCACGTCCGG |
| | Format: | CCCCACCGAGCGAGCGA |
| | t1 | ATGCGCATAGAGGGAGT |
| | | GGCCAA |
| SEQ | source: | TTGGCCACTCCCTGAAT |
| ID | AAV4 | GCGCGCTCGCTCACTCA |
| No: | left; | CTCGGCCCTGGAGACCA |
| 54 | Recogn. | AAGGTCTCCAGACTGCC |
| | Site: | GGCCTCTGGCCGGCAGG |
| | Nb.BsmI; | GCCGAGTGAGTGAGCGA |
| | Format: | GCGCGCATTCAGGGAGT |
| | b1 | GGCCAA |
| SEQ | source: | TTGGCCACTCCCTCTAT |
| ID | AAV4 | GCGCATTCGCTCACTCA |
| No: | left; | CTCGGCCCTGGAGACCA |
| 55 | Recogn. | AAGGTCTCCAGACTGCC |
| | Site: | GGCCTCTGGCCGGCAGG |
| | Nb.BsmI; | GCCGAGTGAGTGAGCGA |
| | Format: | ATGCGCATAGAGGGAGT |
| | t1 | GGCCAA |
| SEQ | source: | TTGGCCACATTAGGAAT |
| ID | AAV4 | GCGCGCTCGCTCACTCA |
| No: | right; | CTCGGCCCTGGAGACCA |
| 56 | Recogn. | AAGGTCTCCAGACTGCC |
| | Site: | GGCCTCTGGCCGGCAGG |
| | Nb.BsmI; | GCCGAGTGAGTGAGCGA |
| | Format: | GCGCGCATTCAGGGAGT |
| | b1 | GGCCAA |
| SEQ | source: | TTGGCCACATTAGCTAT |
| ID | AAV4 | GCGCATTCGCTCACTCA |
| No: | right; | CTCGGCCCTGGAGACCA |
| 57 | Recogn. | AAGGTCTCCAGACTGCC |
| | Site: | GGCCTCTGGCCGGCAGG |
| | Nb.BsmI; | GCCGAGTGAGTGAGCGA |
| | Format: | ATGCGCATAGAGGGAGT |
| | t1 | GGCCAA |
| SEQ | source: | CTCTCCCCGAATGCGCG |
| ID | AAV5; | TTCGCTCGCTCGCTGGC |
| No: | Recogn. | TCGTTTGGGGGGGTGGC |
| 58 | Site: | AGCTCAAAGAGCTGCCA |
| | Nb.BsmI; | GACGACGGCCCTCTGGC |
| | Format: | CGTCGCCCCCCCAAACG |
| | b1 | AGCCAGCGAGCGAGCGA |
| | | ACGCGCATTCGGGGAGA |
| | | G |
| SEQ | source: | CTCTCCCCCCTGTCGCA |
| ID | AAV5; | TTCGCTCGCTCGCTGGC |
| No: | Recogn. | TCGTTTGGGGGGGTGGC |
| 59 | Site: | AGCTCAAAGAGCTGCCA |
| | Nb.BsmI; | GACGACGGCCCTCTGGC |
| | Format: | CGTCGCCCCCCCAAACG |
| | t1 | AGCCAGCGAGCGAGCGA |
| | | ATGCGACAGGGGGGAGA |
| | | G |
| SEQ | source: | TTGGCCACTCCCTGAAT |
| ID | AAV7; | GCGCGCTCGCTCGCTCG |
| No: | Recogn. | GTGGGGCCTGCGGACCA |
| 60 | Site: | AAGGTCCGCAGACGGCA |
| | Nb.BsmI; | GAGCTCTGCTCTGCCGG |
| | Format: | CCCCACCGAGCGAGCGA |
| | b1 | GCGCGCATTCAGGGAGT |
| | | GGCCAA |
| SEQ | source: | TTGGCCACTCCCTCTAT |
| ID | AAV7; | GCGCATTCGCTCGCTCG |
| No: | Recogn. | GTGGGGCCTGCGGACCA |
| 61 | Site: | AAGGTCCGCAGACGGCA |
| | Nb.BsmI; | GAGCTCTGCTCTGCCGG |
| | Format: | CCCCACCGAGCGAGCGA |
| | t1 | ATGCGCATAGAGGGAGT |
| | | GGCCAA |

TABLE 9
Exemplary AAV derived ITRs harboring antiparallel recognition sites for
nicking endonuclease Nb.BsrDI

| SEQ ID No: | Name | Full Sequence |
|---|---|---|
| SEQ ID | source: AAV1; | TTGCCCACTCCCGCAATGCGCGCTCGCTCGCT |
| No: 62 | Recogn. Site: | CGGTGGGGCCTGCGGACCAAAGGTCCGCAGA |
| | Nb.BsrDI; Format: b1 | CGGCAGAGCTCTGCTCTGCCGGCCCCACCGAG |
| | | CGAGCGAGCGCGCATTGCGGGAGTGGGCAA |
| SEQ ID | source: AAV1; | TTGCCCACTCCCTCATTGCGCGCTCGCTCGCT |
| No: 63 | Recogn. Site: | CGGTGGGGCCTGCGGACCAAAGGTCCGCAGA |
| | Nb.BsrDI; Format: t1 | CGGCAGAGCTCTGCTCTGCCGGCCCCACCGAG |
| | | CGAGCGAGCGCGCAATGAGGGAGTGGGCAA |
| SEQ ID | source: AAV2; | TTGGCCACTCCCGCAATGCGCGCTCGCTCGCT |
| No: 64 | Recogn. Site: | CACTGAGGCCGGGCGACCAAAGGTCGCCCGA |
| | Nb.BsrDI; Format: b1 | CGCCCGGGCTTTGCCCGGGCGGCCTCAGTGAG |
| | | CGAGCGAGCGCGCATTGCGGGAGTGGCCAA |
| SEQ ID | source: AAV2; | TTGGCCACTCCCTCATTGCGCGCTCGCTCGCT |
| No: 65 | Recogn. Site: | CACTGAGGCCGGGCGACCAAAGGTCGCCCGA |
| | Nb.BsrDI; Format: t1 | CGCCCGGGCTTTGCCCGGGCGGCCTCAGTGAG |
| | | CGAGCGAGCGCGCAATGAGGGAGTGGCCAA |
| SEQ ID | source: AAV3; | TTGGCCACTCCCGCAATGCGCACTCGCTCGCT |
| No: 66 | Recogn. Site: | CGGTGGGGCCTGGCGACCAAAGGTCGCCAGA |
| | Nb.BsrDI; Format: b1 | CGGACGTGCTTTGCACGTCCGGCCCCACCGAG |
| | | CGAGCGAGTGCGCATTGCGGGAGTGGCCAA |
| SEQ ID | source: AAV3; | TTGGCCACTCCCTCATTGCGCACTCGCTCGCT |
| No: 67 | Recogn. Site: | CGGTGGGGCCTGGCGACCAAAGGTCGCCAGA |
| | Nb.BsrDI; Format: t1 | CGGACGTGCTTTGCACGTCCGGCCCCACCGAG |
| | | CGAGCGAGTGCGCAATGAGGGAGTGGCCAA |
| SEQ ID | source: AAV4_left; | TTGGCCACTCCCGCAATGCGCGCTCGCTCACT |
| No: 68 | Recogn. Site: | CACTCGGCCCTGGAGACCAAAGGTCTCCAGA |
| | Nb.BsrDI; Format: b1 | CTGCCGGCCTCTGGCCGGCAGGGCCGAGTGA |
| | | GTGAGCGAGCGCGCATTGCGGGAGTGGCCAA |
| SEQ ID | source: AAV4_left; | TTGGCCACTCCCTCATTGCGCGCTCGCTCACT |
| No: 69 | Recogn. Site: | CACTCGGCCCTGGAGACCAAAGGTCTCCAGA |
| | Nb.BsrDI; Format: t1 | CTGCCGGCCTCTGGCCGGCAGGGCCGAGTGA |
| | | GTGAGCGAGCGCGCAATGAGGGAGTGGCCAA |
| SEQ ID | source: AAV4_right; | TTGGCCACATTAGCAATGCGCGCTCGCTCACT |
| No: 70 | Recogn. Site: | CACTCGGCCCTGGAGACCAAAGGTCTCCAGA |
| | Nb.BsrDI; Format: b1 | CTGCCGGCCTCTGGCCGGCAGGGCCGAGTGA |
| | | GTGAGCGAGCGCGCATTGCGGGAGTGGCCAA |
| SEQ ID | source: AAV4_right; | TTGGCCACATTAGCATTGCGCGCTCGCTCACT |
| No: 71 | Recogn. Site: | CACTCGGCCCTGGAGACCAAAGGTCTCCAGA |
| | Nb.BsrDI; Format: t1 | CTGCCGGCCTCTGGCCGGCAGGGCCGAGTGA |
| | | GTGAGCGAGCGCGCAATGAGGGAGTGGCCAA |
| SEQ ID | source: AAV5; | CTCTCCGCAATGTCGCGTTCGCTCGCTCGCTG |
| No: 72 | Recogn. Site: | GCTCGTTTGGGGGGGTGGCAGCTCAAAGAGC |
| | Nb.BsrDI; Format: b1 | TGCCAGACGACGGCCCTCTGGCCGTCGCCCCC |
| | | CCAAACGAGCCAGCGAGCGAGCGAACGCGAC |
| | | ATTGCGGAGAG |
| SEQ ID | source: AAV5; | CTCTCCCCCATTGCGCGTTCGCTCGCTCGCTG |
| No: 73 | Recogn. Site: | GCTCGTTTGGGGGGGTGGCAGCTCAAAGAGC |
| | Nb.BsrDI; Format: t1 | TGCCAGACGACGGCCCTCTGGCCGTCGCCCCC |
| | | CCAAACGAGCCAGCGAGCGAGCGAACGCGCA |
| | | ATGGGGGAGAG |
| SEQ ID | source: AAV7; | TTGGCCACTCCCGCAATGCGCGCTCGCTCGCT |
| No: 74 | Recogn. Site: | CGGTGGGGCCTGCGGACCAAAGGTCCGCAGA |
| | Nb.BsrDI; Format: b1 | CGGCAGAGCTCTGCTCTGCCGGCCCCACCGAG |
| | | CGAGCGAGCGCGCATTGCGGGAGTGGCCAA |
| SEQ ID | source: AAV7; | TTGGCCACTCCCTCATTGCGCGCTCGCTCGCT |
| No: 75 | Recogn. Site: | CGGTGGGGCCTGCGGACCAAAGGTCCGCAGA |
| | Nb.BsrDI; Format: t1 | CGGCAGAGCTCTGCTCTGCCGGCCCCACCGAG |
| | | CGAGCGAGCGCGCAATGAGGGAGTGGCCAA |

TABLE 10
Exemplary AAV derived ITRs harboring antiparallel recognition sites for
nicking endonuclease Nb.BssSi

| SEQ ID No: | Name | Full Sequence |
|---|---|---|
| SEQ ID | source: AAV1; | TTGCCCACGAGCTCTCTGCGCGCTCGCTCGCT |
| No: 76 | Recogn. Site: | CGGTGGGGCCTGCGGACCAAAGGTCCGCAGA |
| | Nb.BssSI; Format: b1 | CGGCAGAGCTCTGCTCTGCCGGCCCCACCGAG |
| | | CGAGCGAGCGCGCAGAGAGCTCGTGGGCAA |
| SEQ ID | source: AAV1; | TTGCCCACTCCCTCGTGGCGCGCTCGCTCGCT |
| No: 77 | Recogn. Site: | CGGTGGGGCCTGCGGACCAAAGGTCCGCAGA |
| | Nb.BssSI; Format: t1 | CGGCAGAGCTCTGCTCTGCCGGCCCCACCGAG |
| | | CGAGCGAGCGCGCCACGAGGGAGTGGGCAA |
| SEQ ID | source: AAV2; | TTGGCCACGAGCTCTCTGCGCGCTCGCTCGCT |
| No: 78 | Recogn. Site: | CACTGAGGCCGGGCGACCAAAGGTCGCCCGA |
| | Nb.BssSI; Format: b1 | CGCCCGGGCTTTGCCCGGGCGGCCTCAGTGAG |
| | | CGAGCGAGCGCGCAGAGAGCTCGTGGCCAA |
| SEQ ID | source: AAV2; | TTGGCCACTCCCTCGTGGCGCGCTCGCTCGCT |
| No: 79 | Recogn. Site: | CACTGAGGCCGGGCGACCAAAGGTCGCCCGA |
| | Nb.BssSI; Format: t1 | CGCCCGGGCTTTGCCCGGGCGGCCTCAGTGAG |
| | | CGAGCGAGCGCGCCACGAGGGAGTGGCCAA |
| SEQ ID | source: AAV3; | TTGGCCACGAGCTCTATGCGCACTCGCTCGCT |
| No: 80 | Recogn. Site: | CGGTGGGGCCTGGCGACCAAAGGTCGCCAGA |
| | Nb.BssSI; Format: b1 | CGGACGTGCTTTGCACGTCCGGCCCCACCGAG |
| | | CGAGCGAGTGCGCATAGAGCTCGTGGCCAA |
| SEQ ID | source: AAV3; | TTGGCCACTCCCTCGTGGCGCACTCGCTCGCT |
| No: 81 | Recogn. Site: | CGGTGGGGCCTGGCGACCAAAGGTCGCCAGA |
| | Nb.BssSI; Format: t1 | CGGACGTGCTTTGCACGTCCGGCCCCACCGAG |
| | | CGAGCGAGTGCGCCACGAGGGAGTGGCCAA |
| SEQ ID | source: AAV4_left; | TTGGCCACGAGCTCTATGCGCGCTCGCTCACT |
| No: 82 | Recogn. Site: | CACTCGGCCCTGGAGACCAAAGGTCTCCAGA |
| | Nb.BssSI; Format: b1 | CTGCCGGCCTCTGGCCGGCAGGGCCGAGTGA |
| | | GTGAGCGAGCGCGCATAGAGCTCGTGGCCAA |
| SEQ ID | source: AAV4_left; | TTGGCCACTCCCTCGTGGCGCGCTCGCTCACT |
| No: 83 | Recogn. Site: | CACTCGGCCCTGGAGACCAAAGGTCTCCAGA |
| | Nb.BssSI; Format: t1 | CTGCCGGCCTCTGGCCGGCAGGGCCGAGTGA |
| | | GTGAGCGAGCGCGCCACGAGGGAGTGGCCAA |
| SEQ ID | source: AAV4_right; | TTGGCCACGAGAGCTATGCGCGCTCGCTCACT |
| No: 84 | Recogn. Site: | CACTCGGCCCTGGAGACCAAAGGTCTCCAGA |
| | Nb.BssSI; Format: b1 | CTGCCGGCCTCTGGCCGGCAGGGCCGAGTGA |
| | | GTGAGCGAGCGCGCATAGAGCTCGTGGCCAA |
| SEQ ID | source: AAV4_right; | TTGGCCACATTCTCGTGGCGCGCTCGCTCACT |
| No: 85 | Recogn. Site: | CACTCGGCCCTGGAGACCAAAGGTCTCCAGA |
| | Nb.BssSI; Format: t1 | CTGCCGGCCTCTGGCCGGCAGGGCCGAGTGA |
| | | GTGAGCGAGCGCGCCACGAGGGAGTGGCCAA |
| SEQ ID | source: AAV5; | CTCACGAGCCTGTCGCGTTCGCTCGCTCGCTG |
| No: 86 | Recogn. Site: | GCTCGTTTGGGGGGGTGGCAGCTCAAAGAGC |
| | Nb.BssSI; Format: b1 | TGCCAGACGACGGCCCTCTGGCCGTCGCCCCC |
| | | CCAAACGAGCCAGCGAGCGAGCGAACGCGAC |
| | | AGGCTCGTGAG |
| SEQ ID | source: AAV5; | CTCTCCCTCGTGTCGCGTTCGCTCGCTCGCTG |
| No: 87 | Recogn. Site: | GCTCGTTTGGGGGGGTGGCAGCTCAAAGAGC |
| | Nb.BssSI; Format: t1 | TGCCAGACGACGGCCCTCTGGCCGTCGCCCCC |
| | | CCAAACGAGCCAGCGAGCGAGCGAACGCGAC |
| | | ACGAGGGAGAG |
| SEQ ID | source: AAV7; | TTGGCCACGAGCTCTATGCGCGCTCGCTCGCT |
| No: 88 | Recogn. Site: | CGGTGGGGCCTGCGGACCAAAGGTCCGCAGA |
| | Nb.BssSI; Format: b1 | CGGCAGAGCTCTGCTCTGCCGGCCCCACCGAG |
| | | CGAGCGAGCGCGCATAGAGCTCGTGGCCAA |
| SEQ ID | source: AAV7; | TTGGCCACTCCCTCGTGGCGCGCTCGCTCGCT |
| No: 89 | Recogn. Site: | CGGTGGGGCCTGCGGACCAAAGGTCCGCAGA |
| | Nb.BssSI; Format: t1 | CGGCAGAGCTCTGCTCTGCCGGCCCCACCGAG |
| | | CGAGCGAGCGCGCCACGAGGGAGTGGCCAA |

TABLE 11
Exemplary AAV derived ITRs harboring antiparallel recognition sites for
nicking endonuclease Nb.BtsI:

| SEQ ID No: | Name | Full Sequence |
|---|---|---|
| SEQ ID | source: AAV1; | TTGCCCACTCCCGCAGTGCGCGCTCGCTCGCT |
| No: 90 | Recogn. Site: | CGGTGGGGCCTGCGGACCAAAGGTCCGCAGA |
| | Nb.BtsI; Format: b1 | CGGCAGAGCTCTGCTCTGCCGGCCCCACCGAG |
| | | CGAGCGAGCGCGCACTGCGGGAGTGGGCAA |
| SEQ ID | source: AAV1; | TTGCCCACTCCCTCACTGCGCGCTCGCTCGCT |
| No: 91 | Recogn. Site: | CGGTGGGGCCTGCGGACCAAAGGTCCGCAGA |
| | Nb.BtsI; Format: t1 | CGGCAGAGCTCTGCTCTGCCGGCCCCACCGAG |
| | | CGAGCGAGCGCGCAGTGAGGGAGTGGGCAA |
| SEQ ID | source: AAV2; | TTGGCCACTCCCGCAGTGCGCGCTCGCTCGCT |
| No: 92 | Recogn. Site: | CACTGAGGCCGGGCGACCAAAGGTCGCCCGA |
| | Nb.BtsI; Format: b1 | CGCCCGGGCTTTGCCCGGGCGGCCTCAGTGAG |
| | | CGAGCGAGCGCGCACTGCGGGAGTGGCCAA |
| SEQ ID | source: AAV2; | TTGGCCACTCCCTCACTGCGCGCTCGCTCGCT |
| No: 93 | Recogn. Site: | CACTGAGGCCGGGCGACCAAAGGTCGCCCGA |
| | Nb.BtsI; Format: t1 | CGCCCGGGCTTTGCCCGGGCGGCCTCAGTGAG |
| | | CGAGCGAGCGCGCAGTGAGGGAGTGGCCAA |
| SEQ ID | source: AAV3; | TTGGCCACTCCCGCAGTGCGCACTCGCTCGCT |
| No: 94 | Recogn. Site: | CGGTGGGGCCTGGCGACCAAAGGTCGCCAGA |
| | Nb.BtsI; Format: b1 | CGGACGTGCTTTGCACGTCCGGCCCCACCGAG |
| | | CGAGCGAGTGCGCACTGCGGGAGTGGCCAA |
| SEQ ID | source: AAV3; | TTGGCCACTCCCTCACTGCGCACTCGCTCGCT |
| No: 95 | Recogn. Site: | CGGTGGGGCCTGGCGACCAAAGGTCGCCAGA |
| | Nb.BtsI; Format: t1 | CGGACGTGCTTTGCACGTCCGGCCCCACCGAG |
| | | CGAGCGAGTGCGCAGTGAGGGAGTGGCCAA |
| SEQ ID | source: AAV4_left; | TTGGCCACTCCCGCAGTGCGCGCTCGCTCACT |
| No: 96 | Recogn. Site: | CACTCGGCCCTGGAGACCAAAGGTCTCCAGA |
| | Nb.BtsI; Format: b1 | CTGCCGGCCTCTGGCCGGCAGGGCCGAGTGA |
| | | GTGAGCGAGCGCGCACTGCGGGAGTGGCCAA |
| SEQ ID | source: AAV4_left; | TTGGCCACTCCCTCACTGCGCGCTCGCTCACT |
| No: 97 | Recogn. Site: | CACTCGGCCCTGGAGACCAAAGGTCTCCAGA |
| | Nb.BtsI; Format: t1 | CTGCCGGCCTCTGGCCGGCAGGGCCGAGTGA |
| | | GTGAGCGAGCGCGCAGTGAGGGAGTGGCCAA |
| SEQ ID | source: AAV4_right; | TTGGCCACATTAGCAGTGCGCGCTCGCTCACT |
| No: 98 | Recogn. Site: | CACTCGGCCCTGGAGACCAAAGGTCTCCAGA |
| | Nb.BtsI; Format: b1 | CTGCCGGCCTCTGGCCGGCAGGGCCGAGTGA |
| | | GTGAGCGAGCGCGCACTGCGGGAGTGGCCAA |
| SEQ ID | source: AAV4_right; | TTGGCCACATTAGCACTGCGCGCTCGCTCACT |
| No: 99 | Recogn. Site: | CACTCGGCCCTGGAGACCAAAGGTCTCCAGA |
| | Nb.BtsI; Format: t1 | CTGCCGGCCTCTGGCCGGCAGGGCCGAGTGA |
| | | GTGAGCGAGCGCGCAGTGAGGGAGTGGCCAA |
| SEQ ID | source: AAV5; | CTCTCCGCAGTGTCGCGTTCGCTCGCTCGCTG |
| No: 100 | Recogn. Site: | GCTCGTTTGGGGGGGTGGCAGCTCAAAGAGC |
| | Nb.BtsI; Format: b1 | TGCCAGACGACGGCCCTCTGGCCGTCGCCCCC |
| | | CCAAACGAGCCAGCGAGCGAGCGAACGCGAC |
| | | ACTGCGGAGAG |
| SEQ ID | source: AAV5; | CTCTCCCCACTGCCGCGTTCGCTCGCTCGCTG |
| No: 101 | Recogn. Site: | GCTCGTTTGGGGGGGTGGCAGCTCAAAGAGC |
| | Nb.BtsI; Format: t1 | TGCCAGACGACGGCCCTCTGGCCGTCGCCCCC |
| | | CCAAACGAGCCAGCGAGCGAGCGAACGCGGC |
| | | AGTGGGGAGAG |
| SEQ ID | source: AAV7; | TTGGCCACTCCCGCAGTGCGCGCTCGCTCGCT |
| No: 102 | Recogn. Site: | CGGTGGGGCCTGCGGACCAAAGGTCCGCAGA |
| | Nb.BtsI; Format: b1 | CGGCAGAGCTCTGCTCTGCCGGCCCCACCGAG |
| | | CGAGCGAGCGCGCACTGCGGGAGTGGCCAA |
| SEQ ID | source: AAV7; | TTGGCCACTCCCTCACTGCGCGCTCGCTCGCT |
| No: 103 | Recogn. Site: | CGGTGGGGCCTGCGGACCAAAGGTCCGCAGA |
| | Nb.BtsI; Format: t1 | CGGCAGAGCTCTGCTCTGCCGGCCCCACCGAG |
| | | CGAGCGAGCGCGCAGTGAGGGAGTGGCCAA |

TABLE 12
Exemplary AAV derived ITRs harboring antiparallel recognition sites for
nicking endonuclease Nt.AlwI:

| SEQ ID No: | Name | Full Sequence |
|---|---|---|
| SEQ ID | source: AAV1; | TTGCCCACTCCCTCGATCCGCGCTCGCTCGCT |
| No: 104 | Recogn. Site: | CGGTGGGGCCTGCGGACCAAAGGTCCGCAGA |
| | Nt.AlwI; Format: b1 | CGGCAGAGCTCTGCTCTGCCGGCCCCACCGAG |
| | | CGAGCGAGCGCGGATCGAGGGAGTGGGCAA |
| SEQ ID | source: AAV1; | TTGCCCACTGGATCTCTGCGCGCTCGCTCGCT |
| No: 105 | Recogn. Site: | CGGTGGGGCCTGCGGACCAAAGGTCCGCAGA |
| | Nt.AlwI; Format: t1 | CGGCAGAGCTCTGCTCTGCCGGCCCCACCGAG |
| | | CGAGCGAGCGCGCAGAGATCCAGTGGGCAA |
| SEQ ID | source: AAV2; | TTGGCCACTCCCTCGATCCGCGCTCGCTCGCT |
| No: 106 | Recogn. Site: | CACTGAGGCCGGGCGACCAAAGGTCGCCCGA |
| | Nt.AlwI; Format: b1 | CGCCCGGGCTTTGCCCGGGCGGCCTCAGTGAG |
| | | CGAGCGAGCGCGGATCGAGGGAGTGGCCAA |
| SEQ ID | source: AAV2; | TTGGCCACTGGATCTCTGCGCGCTCGCTCGCT |
| No: 107 | Recogn. Site: | CACTGAGGCCGGGCGACCAAAGGTCGCCCGA |
| | Nt.AlwI; Format: t1 | CGCCCGGGCTTTGCCCGGGCGGCCTCAGTGAG |
| | | CGAGCGAGCGCGCAGAGATCCAGTGGCCAA |
| SEQ ID | source: AAV3; | TTGGCCACTCCCTCGATCCGCACTCGCTCGCT |
| No: 108 | Recogn. Site: | CGGTGGGGCCTGGCGACCAAAGGTCGCCAGA |
| | Nt.AlwI; Format: b1 | CGGACGTGCTTTGCACGTCCGGCCCCACCGAG |
| | | CGAGCGAGTGCGGATCGAGGGAGTGGCCAA |
| SEQ ID | source: AAV3; | TTGGCCACTGGATCTATGCGCACTCGCTCGCT |
| No: 109 | Recogn. Site: | CGGTGGGGCCTGGCGACCAAAGGTCGCCAGA |
| | Nt.AlwI; Format: t1 | CGGACGTGCTTTGCACGTCCGGCCCCACCGAG |
| | | CGAGCGAGTGCGCATAGATCCAGTGGCCAA |
| SEQ ID | source: AAV4_left; | TTGGCCACTCCCTCGATCCGCGCTCGCTCACT |
| No: 110 | Recogn. Site: | CACTCGGCCCTGGAGACCAAAGGTCTCCAGA |
| | Nt.AlwI; Format: b1 | CTGCCGGCCTCTGGCCGGCAGGGCCGAGTGA |
| | | GTGAGCGAGCGCGGATCGAGGGAGTGGCCAA |
| SEQ ID | source: AAV4_left; | TTGGCCACTGGATCTATGCGCGCTCGCTCACT |
| No: 111 | Recogn. Site: | CACTCGGCCCTGGAGACCAAAGGTCTCCAGA |
| | Nt.AlwI; Format: t1 | CTGCCGGCCTCTGGCCGGCAGGGCCGAGTGA |
| | | GTGAGCGAGCGCGCATAGATCCAGTGGCCAA |
| SEQ ID | source: AAV4_right; | TTGGCCACATTAGCGATCCGCGCTCGCTCACT |
| No: 112 | Recogn. Site: | CACTCGGCCCTGGAGACCAAAGGTCTCCAGA |
| | Nt.AlwI; Format: b1 | CTGCCGGCCTCTGGCCGGCAGGGCCGAGTGA |
| | | GTGAGCGAGCGCGGATCGAGGGAGTGGCCAA |
| SEQ ID | source: AAV4_right; | TTGGCCACAGGATCTATGCGCGCTCGCTCACT |
| No: 113 | Recogn. Site: | CACTCGGCCCTGGAGACCAAAGGTCTCCAGA |
| | Nt.AlwI; Format: t1 | CTGCCGGCCTCTGGCCGGCAGGGCCGAGTGA |
| | | GTGAGCGAGCGCGCATAGATCCAGTGGCCAA |
| SEQ ID | source: AAV5; | CTCTCCCCCCTGTCGCGATCCCTCGCTCGCTG |
| No: 114 | Recogn. Site: | GCTCGTTTGGGGGGGTGGCAGCTCAAAGAGC |
| | Nt.AlwI; Format: b1 | TGCCAGACGACGGCCCTCTGGCCGTCGCCCCC |
| | | CCAAACGAGCCAGCGAGCGAGGGATCGCGAC |
| | | AGGGGGGAGAG |
| SEQ ID | source: AAV5; | CTCTCCCCCGGATCGCGTTCGCTCGCTCGCTG |
| No: 115 | Recogn. Site: | GCTCGTTTGGGGGGGTGGCAGCTCAAAGAGC |
| | Nt.AlwI; Format: t1 | TGCCAGACGACGGCCCTCTGGCCGTCGCCCCC |
| | | CCAAACGAGCCAGCGAGCGAGCGAACGCGAT |
| | | CCGGGGGAGAG |
| SEQ ID | source: AAV7; | TTGGCCACTCCCTCGATCCGCGCTCGCTCGCT |
| No: 116 | Recogn. Site: | CGGTGGGGCCTGCGGACCAAAGGTCCGCAGA |
| | Nt.AlwI; Format: b1 | CGGCAGAGCTCTGCTCTGCCGGCCCCACCGAG |
| | | CGAGCGAGCGCGGATCGAGGGAGTGGCCAA |
| SEQ ID | source: AAV7; | TTGGCCACTGGATCTATGCGCGCTCGCTCGCT |
| No: 117 | Recogn. Site: | CGGTGGGGCCTGCGGACCAAAGGTCCGCAGA |
| | Nt.AlwI; Format: t1 | CGGCAGAGCTCTGCTCTGCCGGCCCCACCGAG |
| | | CGAGCGAGCGCGCATAGATCCAGTGGCCAA |

TABLE 13
Exemplary AAV derived ITRs harboring antiparallel recognition sites for
nicking endonuclease Nt.BbvCI:

| SEQ ID No: | Name | Full Sequence |
|---|---|---|
| SEQ ID | source: AAV1; | TTGCCCACTCCCGCTGAGGGCGCTCGCTCGCT |
| No: 118 | Recogn. Site: | CGGTGGGGCCTGCGGACCAAAGGTCCGCAGA |
| | Nt.BbvCI; Format: b1 | CGGCAGAGCTCTGCTCTGCCGGCCCCACCGAG |
| | | CGAGCGAGCGCCCTCAGCGGGAGTGGGCAA |
| SEQ ID | source: AAV1; | TTGCCCACTCCCCCTCAGCGCGCTCGCTCGCT |
| No: 119 | Recogn. Site: | CGGTGGGGCCTGCGGACCAAAGGTCCGCAGA |
| | Nt.BbvCI; Format: t1 | CGGCAGAGCTCTGCTCTGCCGGCCCCACCGAG |
| | | CGAGCGAGCGCGCTGAGGGGGAGTGGGCAA |
| SEQ ID | source: AAV2; | TTGGCCACTCCCGCTGAGGGCGCTCGCTCGCT |
| No: 120 | Recogn. Site: | CACTGAGGCCGGGCGACCAAAGGTCGCCCGA |
| | Nt.BbvCI; Format: b1 | CGCCCGGGCTTTGCCCGGGCGGCCTCAGTGAG |
| | | CGAGCGAGCGCCCTCAGCGGGAGTGGCCAA |
| SEQ ID | source: AAV2; | TTGGCCACTCCCCCTCAGCGCGCTCGCTCGCT |
| No: 121 | Recogn. Site: | CACTGAGGCCGGGCGACCAAAGGTCGCCCGA |
| | Nt.BbvCI; Format: t1 | CGCCCGGGCTTTGCCCGGGCGGCCTCAGTGAG |
| | | CGAGCGAGCGCGCTGAGGGGGAGTGGCCAA |
| SEQ ID | source: AAV3; | TTGGCCACTCCCGCTGAGGGCACTCGCTCGCT |
| No: 122 | Recogn. Site: | CGGTGGGGCCTGGCGACCAAAGGTCGCCAGA |
| | Nt.BbvCI; Format: b1 | CGGACGTGCTTTGCACGTCCGGCCCCACCGAG |
| | | CGAGCGAGTGCCCTCAGCGGGAGTGGCCAA |
| SEQ ID | source: AAV3; | TTGGCCACTCCCCCTCAGCGCACTCGCTCGCT |
| No: 123 | Recogn. Site: | CGGTGGGGCCTGGCGACCAAAGGTCGCCAGA |
| | Nt.BbvCI; Format: t1 | CGGACGTGCTTTGCACGTCCGGCCCCACCGAG |
| | | CGAGCGAGTGCGCTGAGGGGGAGTGGCCAA |
| SEQ ID | source: AAV4_left; | TTGGCCACTCCCGCTGAGGGCGCTCGCTCACT |
| No: 124 | Recogn. Site: | CACTCGGCCCTGGAGACCAAAGGTCTCCAGA |
| | Nt.BbvCI; Format: b1 | CTGCCGGCCTCTGGCCGGCAGGGCCGAGTGA |
| | | GTGAGCGAGCGCCCTCAGCGGGAGTGGCCAA |
| SEQ ID | source: AAV4_left; | TTGGCCACTCCCCCTCAGCGCGCTCGCTCACT |
| No: 125 | Recogn. Site: | CACTCGGCCCTGGAGACCAAAGGTCTCCAGA |
| | Nt.BbvCI; Format: t1 | CTGCCGGCCTCTGGCCGGCAGGGCCGAGTGA |
| | | GTGAGCGAGCGCGCTGAGGGGGAGTGGCCAA |
| SEQ ID | source: AAV4_right; | TTGGCCACATTAGCTGAGGGCGCTCGCTCACT |
| No: 126 | Recogn. Site: | CACTCGGCCCTGGAGACCAAAGGTCTCCAGA |
| | Nt.BbvCI; Format: b1 | CTGCCGGCCTCTGGCCGGCAGGGCCGAGTGA |
| | | GTGAGCGAGCGCCCTCAGCGGGAGTGGCCAA |
| SEQ ID | source: AAV4_right; | TTGGCCACATTACCTCAGCGCGCTCGCTCACT |
| No: 127 | Recogn. Site: | CACTCGGCCCTGGAGACCAAAGGTCTCCAGA |
| | Nt.BbvCI; Format: t1 | CTGCCGGCCTCTGGCCGGCAGGGCCGAGTGA |
| | | GTGAGCGAGCGCGCTGAGGGGGAGTGGCCAA |
| SEQ ID | source: AAV5; | CTCTCCCCGCTGAGGCGTTCGCTCGCTCGCTG |
| No: 128 | Recogn. Site: | GCTCGTTTGGGGGGGTGGCAGCTCAAAGAGC |
| | Nt.BbvCI; Format: b1 | TGCCAGACGACGGCCCTCTGGCCGTCGCCCCC |
| | | CCAAACGAGCCAGCGAGCGAGCGAACGCCTC |
| | | AGCGGGGAGAG |
| SEQ ID | source: AAV5; | CTCTCCCCTCAGCCGCGTTCGCTCGCTCGCTG |
| No: 129 | Recogn. Site: | GCTCGTTTGGGGGGGTGGCAGCTCAAAGAGC |
| | Nt.BbvCI; Format: t1 | TGCCAGACGACGGCCCTCTGGCCGTCGCCCCC |
| | | CCAAACGAGCCAGCGAGCGAGCGAACGCGGC |
| | | TGAGGGGAGAG |
| SEQ ID | source: AAV7; | TTGGCCACTCCCGCTGAGGGCGCTCGCTCGCT |
| No: 130 | Recogn. Site: | CGGTGGGGCCTGCGGACCAAAGGTCCGCAGA |
| | Nt.BbvCI; Format: b1 | CGGCAGAGCTCTGCTCTGCCGGCCCCACCGAG |
| | | CGAGCGAGCGCCCTCAGCGGGAGTGGCCAA |
| SEQ ID | source: AAV7; | TTGGCCACTCCCCCTCAGCGCGCTCGCTCGCT |
| No: 131 | Recogn. Site: | CGGTGGGGCCTGCGGACCAAAGGTCCGCAGA |
| | Nt.BbvCI; Format: t1 | CGGCAGAGCTCTGCTCTGCCGGCCCCACCGAG |
| | | CGAGCGAGCGCGCTGAGGGGGAGTGGCCAA |

TABLE 14
Exemplary AAV derived ITRs harboring antiparallel recognition sites for
nicking endonuclease Nt.BsmAI:

| SEQ ID No: | Name | Full Sequence |
|---|---|---|
| SEQ ID | source: AAV1; | TTGCCCACTGAGACTCTGCGCGCTCGCTCGCT |
| No: 132 | Recogn. Site: | CGGTGGGGCCTGCGGACCAAAGGTCCGCAGA |
| | Nt.BsmAI; Format: | CGGCAGAGCTCTGCTCTGCCGGCCCCACCGAG |
| | b1 | CGAGCGAGCGCGCAGAGTCTCAGTGGGCAA |
| SEQ ID | source: AAV1; | TTGCCCACTCCGTCTCTGCGCGCTCGCTCGCT |
| No: 133 | Recogn. Site: | CGGTGGGGCCTGCGGACCAAAGGTCCGCAGA |
| | Nt.BsmAI; Format: t1 | CGGCAGAGCTCTGCTCTGCCGGCCCCACCGAG |
| | | CGAGCGAGCGCGCAGAGACGGAGTGGGCAA |
| SEQ ID | source: AAV2; | TTGGCCACTGAGACTCTGCGCGCTCGCTCGCT |
| No: 134 | Recogn. Site: | CACTGAGGCCGGGCGACCAAAGGTCGCCCGA |
| | Nt.BsmAI; Format: | CGCCCGGGCTTTGCCCGGGCGGCCTCAGTGAG |
| | b1 | CGAGCGAGCGCGCAGAGTCTCAGTGGCCAA |
| SEQ ID | source: AAV2; | TTGGCCACTCCGTCTCTGCGCGCTCGCTCGCT |
| No: 135 | Recogn. Site: | CACTGAGGCCGGGCGACCAAAGGTCGCCCGA |
| | Nt.BsmAI; Format: t1 | CGCCCGGGCTTTGCCCGGGCGGCCTCAGTGAG |
| | | CGAGCGAGCGCGCAGAGACGGAGTGGCCAA |
| SEQ ID | source: AAV3; | TTGGCCACTGAGACTATGCGCACTCGCTCGCT |
| No: 136 | Recogn. Site: | CGGTGGGGCCTGGCGACCAAAGGTCGCCAGA |
| | Nt.BsmAI; Format: | CGGACGTGCTTTGCACGTCCGGCCCCACCGAG |
| | b1 | CGAGCGAGTGCGCATAGTCTCAGTGGCCAA |
| SEQ ID | source: AAV3; | TTGGCCACTCCGTCTCTGCGCACTCGCTCGCT |
| No: 137 | Recogn. Site: | CGGTGGGGCCTGGCGACCAAAGGTCGCCAGA |
| | Nt.BsmAI; Format: t1 | CGGACGTGCTTTGCACGTCCGGCCCCACCGAG |
| | | CGAGCGAGTGCGCAGAGACGGAGTGGCCAA |
| SEQ ID | source: AAV4_left; | TTGGCCACTGAGACTATGCGCGCTCGCTCACT |
| No: 138 | Recogn. Site: | CACTCGGCCCTGGAGACCAAAGGTCTCCAGA |
| | Nt.BsmAI; Format: | CTGCCGGCCTCTGGCCGGCAGGGCCGAGTGA |
| | b1 | GTGAGCGAGCGCGCATAGTCTCAGTGGCCAA |
| SEQ ID | source: AAV4_left; | TTGGCCACTCCGTCTCTGCGCGCTCGCTCACT |
| No: 139 | Recogn. Site: | CACTCGGCCCTGGAGACCAAAGGTCTCCAGA |
| | Nt.BsmAI; Format: t1 | CTGCCGGCCTCTGGCCGGCAGGGCCGAGTGA |
| | | GTGAGCGAGCGCGCAGAGACGGAGTGGCCAA |
| SEQ ID | source: AAV4_right; | TTGGCCACAGAGACTATGCGCGCTCGCTCACT |
| No: 140 | Recogn. Site: | CACTCGGCCCTGGAGACCAAAGGTCTCCAGA |
| | Nt.BsmAI; Format: | CTGCCGGCCTCTGGCCGGCAGGGCCGAGTGA |
| | b1 | GTGAGCGAGCGCGCATAGTCTCAGTGGCCAA |
| SEQ ID | source: AAV4_right; | TTGGCCACATTGTCTCTGCGCGCTCGCTCACT |
| No: 141 | Recogn. Site: | CACTCGGCCCTGGAGACCAAAGGTCTCCAGA |
| | Nt.BsmAI; Format: t1 | CTGCCGGCCTCTGGCCGGCAGGGCCGAGTGA |
| | | GTGAGCGAGCGCGCAGAGACGGAGTGGCCAA |
| SEQ ID | source: AAV5; | CTCTCCCCCGAGACGCGTTCGCTCGCTCGCTG |
| No: 142 | Recogn. Site: | GCTCGTTTGGGGGGGTGGCAGCTCAAAGAGC |
| | Nt.BsmAI; Format: | TGCCAGACGACGGCCCTCTGGCCGTCGCCCCC |
| | b1 | CCAAACGAGCCAGCGAGCGAGCGAACGCGTC |
| | | TCGGGGGAGAG |
| SEQ ID | source: AAV5; | CTCTCCCCCGTCTCGCGTTCGCTCGCTCGCTG |
| No: 143 | Recogn. Site: | GCTCGTTTGGGGGGGTGGCAGCTCAAAGAGC |
| | Nt.BsmAI; Format: t1 | TGCCAGACGACGGCCCTCTGGCCGTCGCCCCC |
| | | CCAAACGAGCCAGCGAGCGAGCGAACGCGAG |
| | | ACGGGGGAGAG |
| SEQ ID | source: AAV7; | TTGGCCACTGAGACTATGCGCGCTCGCTCGCT |
| No: 144 | Recogn. Site: | CGGTGGGGCCTGCGGACCAAAGGTCCGCAGA |
| | Nt.BsmAI; Format: b1 | CGGCAGAGCTCTGCTCTGCCGGCCCCACCGAG |
| | | CGAGCGAGCGCGCATAGTCTCAGTGGCCAA |
| SEQ ID | source: AAV7; | TTGGCCACTCCGTCTCTGCGCGCTCGCTCGCT |
| No: 145 | Recogn. Site: | CGGTGGGGCCTGCGGACCAAAGGTCCGCAGA |
| | Nt.BsmAI; Format: t1 | CGGCAGAGCTCTGCTCTGCCGGCCCCACCGAG |
| | | CGAGCGAGCGCGCAGAGACGGAGTGGCCAA |

TABLE 15
Exemplary AAV derived ITRs harboring antiparallel recognition sites for
nicking endonuclease Nt.BspQI:

| SEQ ID No: | Name | Full Sequence |
|---|---|---|
| SEQ ID | source: AAV1; | TTGCCCACTCCCGAAGAGCGCGCTCGCTCGCT |
| No: 146 | Recogn. Site: | CGGTGGGGCCTGCGGACCAAAGGTCCGCAGA |
| | Nt.BspQI; Format: b1 | CGGCAGAGCTCTGCTCTGCCGGCCCCACCGAG |
| | | CGAGCGAGCGCGCTCTTCGGGAGTGGGCAA |
| SEQ ID | source: AAV1; | TTGCCCACTCCCGCTCTTCGCGCTCGCTCGCTC |
| No: 147 | Recogn. Site: | GGTGGGGCCTGCGGACCAAAGGTCCGCAGAC |
| | Nt.BspQI; Format: t1 | GGCAGAGCTCTGCTCTGCCGGCCCCACCGAGC |
| | | GAGCGAGCGCGAAGAGCGGGAGTGGGCAA |
| SEQ ID | source: AAV2; | TTGGCCACTCCCGAAGAGCGCGCTCGCTCGCT |
| No: 148 | Recogn. Site: | CACTGAGGCCGGGCGACCAAAGGTCGCCCGA |
| | Nt.BspQI; Format: b1 | CGCCCGGGCTTTGCCCGGGCGGCCTCAGTGAG |
| | | CGAGCGAGCGCGCTCTTCGGGAGTGGCCAA |
| SEQ ID | source: AAV2; | TTGGCCACTCCCGCTCTTCGCGCTCGCTCGCT |
| No: 149 | Recogn. Site: | CACTGAGGCCGGGCGACCAAAGGTCGCCCGA |
| | Nt.BspQI; Format: t1 | CGCCCGGGCTTTGCCCGGGCGGCCTCAGTGAG |
| | | CGAGCGAGCGCGAAGAGCGGGAGTGGCCAA |
| SEQ ID | source: AAV3; | TTGGCCACTCCCGAAGAGCGCACTCGCTCGCT |
| No: 150 | Recogn. Site: | CGGTGGGGCCTGGCGACCAAAGGTCGCCAGA |
| | Nt.BspQI; Format: b1 | CGGACGTGCTTTGCACGTCCGGCCCCACCGAG |
| | | CGAGCGAGTGCGCTCTTCGGGAGTGGCCAA |
| SEQ ID | source: AAV3; | TTGGCCACTCCCGCTCTTCGCACTCGCTCGCT |
| No: 151 | Recogn. Site: | CGGTGGGGCCTGGCGACCAAAGGTCGCCAGA |
| | Nt.BspQI; Format: t1 | CGGACGTGCTTTGCACGTCCGGCCCCACCGAG |
| | | CGAGCGAGTGCGAAGAGCGGGAGTGGCCAA |
| SEQ ID | source: AAV4_left; | TTGGCCACTCCCGAAGAGCGCGCTCGCTCACT |
| No: 152 | Recogn. Site: | CACTCGGCCCTGGAGACCAAAGGTCTCCAGA |
| | Nt.BspQI; Format: b1 | CTGCCGGCCTCTGGCCGGCAGGGCCGAGTGA |
| | | GTGAGCGAGCGCGCTCTTCGGGAGTGGCCAA |
| SEQ ID | source: AAV4_left; | TTGGCCACTCCCGCTCTTCGCGCTCGCTCACT |
| No: 153 | Recogn. Site: | CACTCGGCCCTGGAGACCAAAGGTCTCCAGA |
| | Nt.BspQI; Format: t1 | CTGCCGGCCTCTGGCCGGCAGGGCCGAGTGA |
| | | GTGAGCGAGCGCGAAGAGCGGGAGTGGCCAA |
| SEQ ID | source: AAV4_right; | TTGGCCACATTAGAAGAGCGCGCTCGCTCACT |
| No: 154 | Recogn. Site: | CACTCGGCCCTGGAGACCAAAGGTCTCCAGA |
| | Nt.BspQI; Format: b1 | CTGCCGGCCTCTGGCCGGCAGGGCCGAGTGA |
| | | GTGAGCGAGCGCGCTCTTCGGGAGTGGCCAA |
| SEQ ID | source: AAV4_right; | TTGGCCACATTAGCTCTTCGCGCTCGCTCACT |
| No: 155 | Recogn. Site: | CACTCGGCCCTGGAGACCAAAGGTCTCCAGA |
| | Nt.BspQI; Format: t1 | CTGCCGGCCTCTGGCCGGCAGGGCCGAGTGA |
| | | GTGAGCGAGCGCGAAGAGCGGGAGTGGCCAA |
| SEQ ID | source: AAV5; | CTCTCCCGAAGAGCGCGTTCGCTCGCTCGCTG |
| No: 156 | Recogn. Site: | GCTCGTTTGGGGGGGTGGCAGCTCAAAGAGC |
| | Nt.BspQI; Format: b1 | TGCCAGACGACGGCCCTCTGGCCGTCGCCCCC |
| | | CCAAACGAGCCAGCGAGCGAGCGAACGCGCT |
| | | CTTCGGGAGAG |
| SEQ ID | source: AAV5; | CTCTCCCGCTCTTCGCGTTCGCTCGCTCGCTGG |
| No: 157 | Recogn. Site: | CTCGTTTGGGGGGGTGGCAGCTCAAAGAGCT |
| | Nt.BspQI; Format: t1 | GCCAGACGACGGCCCTCTGGCCGTCGCCCCCC |
| | | CAAACGAGCCAGCGAGCGAGCGAACGCGAAG |
| | | AGCGGGAGAG |
| SEQ ID | source: AAV7; | TTGGCCACTCCCGAAGAGCGCGCTCGCTCGCT |
| No: 158 | Recogn. Site: | CGGTGGGGCCTGCGGACCAAAGGTCCGCAGA |
| | Nt.BspQI; Format: b1 | CGGCAGAGCTCTGCTCTGCCGGCCCCACCGAG |
| | | CGAGCGAGCGCGCTCTTCGGGAGTGGCCAA |
| SEQ ID | source: AAV7; | TTGGCCACTCCCGCTCTTCGCGCTCGCTCGCT |
| No: 159 | Recogn. Site: | CGGTGGGGCCTGCGGACCAAAGGTCCGCAGA |
| | Nt.BspQI; Format: t1 | CGGCAGAGCTCTGCTCTGCCGGCCCCACCGAG |
| | | CGAGCGAGCGCGAAGAGCGGGAGTGGCCAA |

TABLE 16
Exemplary AAV derived ITRs harboring antiparallel recognition sites for
nicking endonuclease Nt.BstNBI:

| SEQ ID No: | Name | Full Sequence |
|---|---|---|
| SEQ ID | source: AAV1; | TTGCCCACTCCCTCTCTGCGCGACTCGCTCGC |
| No: 160 | Recogn. Site: | TCGGTGGGGCCTGCGGACCAAAGGTCCGCAG |
| | Nt.BstNBI; Format: | ACGGCAGAGCTCTGCTCTGCCGGCCCCACCGA |
| | b1 | GCGAGCGAGTCGCGCAGAGAGGGAGTGGGCAA |
| SEQ ID | source: AAV1; | TTGCCGAGTCCCTCTCTGCGCGCTCGCTCGCT |
| No: 161 | Recogn. Site: | CGGTGGGGCCTGCGGACCAAAGGTCCGCAGA |
| | Nt.BstNBI; Format: | CGGCAGAGCTCTGCTCTGCCGGCCCCACCGAG |
| | t1 | CGAGCGAGCGCGCAGAGAGGGACTCGGCAA |
| SEQ ID | source: AAV2; | TTGGCCACTCCCTCTCTGCGCGACTCGCTCGC |
| No: 162 | Recogn. Site: | TCACTGAGGCCGGGCGACCAAAGGTCGCCCG |
| | Nt.BstNBI; Format: | ACGCCCGGGCTTTGCCCGGGCGGCCTCAGTGA |
| | b1 | GCGAGCGAGTCGCGCAGAGAGGGAGTGGCCAA |
| SEQ ID | source: AAV2; | TTGGCGAGTCCCTCTCTGCGCGCTCGCTCGCT |
| No: 163 | Recogn. Site: | CACTGAGGCCGGGCGACCAAAGGTCGCCCGA |
| | Nt.BstNBI; Format: | CGCCCGGGCTTTGCCCGGGCGGCCTCAGTGAG |
| | t1 | CGAGCGAGCGCGCAGAGAGGGACTCGCCAA |
| SEQ ID | source: AAV3; | TTGGCCACTCCCTCTATGCGCGACTCGCTCGC |
| No: 164 | Recogn. Site: | TCGGTGGGGCCTGGCGACCAAAGGTCGCCAG |
| | Nt.BstNBI; Format: | ACGGACGTGCTTTGCACGTCCGGCCCCACCGA |
| | b1 | GCGAGCGAGTCGCGCATAGAGGGAGTGGCCAA |
| SEQ ID | source: AAV3; | TTGGCGAGTCCCTCTATGCGCACTCGCTCGCT |
| No: 165 | Recogn. Site: | CGGTGGGGCCTGGCGACCAAAGGTCGCCAGA |
| | Nt.BstNBI; Format: | CGGACGTGCTTTGCACGTCCGGCCCCACCGAG |
| | t1 | CGAGCGAGTGCGCATAGAGGGACTCGCCAA |
| SEQ ID | source: AAV4_left; | TTGGCCACTCCCTCTATGCGCGACTCGCTCAC |
| No: 166 | Recogn. Site: | TCACTCGGCCCTGGAGACCAAAGGTCTCCAG |
| | Nt.BstNBI; Format: | ACTGCCGGCCTCTGGCCGGCAGGGCCGAGTG |
| | b1 | AGTGAGCGAGTCGCGCATAGAGGGAGTGGCC |
| | | AA |
| SEQ ID | source: AAV4_left; | TTGGCGAGTCCCTCTATGCGCGCTCGCTCACT |
| No: 167 | Recogn. Site: | CACTCGGCCCTGGAGACCAAAGGTCTCCAGA |
| | Nt.BstNBI; Format: | CTGCCGGCCTCTGGCCGGCAGGGCCGAGTGA |
| | t1 | GTGAGCGAGCGCGCATAGAGGGACTCGCCAA |
| SEQ ID | source: AAV4_right; | TTGGCCACATTAGCTATGCGCGACTCGCTCAC |
| No: 168 | Recogn. Site: | TCACTCGGCCCTGGAGACCAAAGGTCTCCAG |
| | Nt.BstNBI; Format: | ACTGCCGGCCTCTGGCCGGCAGGGCCGAGTG |
| | b1 | AGTGAGCGAGTCGCGCATAGAGGGAGTGGCC |
| | | AA |
| SEQ ID | source: AAV4_right; | TTGGCCAGAGTCGCTATGCGCGCTCGCTCACT |
| No: 169 | Recogn. Site: | CACTCGGCCCTGGAGACCAAAGGTCTCCAGA |
| | Nt.BstNBI; Format: | CTGCCGGCCTCTGGCCGGCAGGGCCGAGTGA |
| | t1 | GTGAGCGAGCGCGCATAGAGACTCTGGCCAA |
| SEQ ID | source: AAV5; | CTCTCCCCCCTGTCGCGACTCGCTCGCTCGCT |
| No: 170 | Recogn. Site: | GGCTCGTTTGGGGGGGTGGCAGCTCAAAGAG |
| | Nt.BstNBI; Format: | CTGCCAGACGACGGCCCTCTGGCCGTCGCCCC |
| | b1 | CCCAAACGAGCCAGCGAGCGAGCGAGTCGCG |
| | | ACAGGGGGGAGAG |
| SEQ ID | source: AAV5; | CTCTCCCCCGAGTCGCGTTCGCTCGCTCGCTG |
| No: 171 | Recogn. Site: | GCTCGTTTGGGGGGGTGGCAGCTCAAAGAGC |
| | Nt.BstNBI; Format: | TGCCAGACGACGGCCCTCTGGCCGTCGCCCCC |
| | t1 | CCAAACGAGCCAGCGAGCGAGCGAACGCGAC |
| | | TCGGGGGAGAG |
| SEQ ID | source: AAV7; | TTGGCCACTCCCTCTATGCGCGACTCGCTCGC |
| No: 172 | Recogn. Site: | TCGGTGGGGCCTGCGGACCAAAGGTCCGCAG |
| | Nt.BstNBI; Format: | ACGGCAGAGCTCTGCTCTGCCGGCCCCACCGA |
| | b1 | GCGAGCGAGTCGCGCATAGAGGGAGTGGCCAA |
| SEQ ID | source: AAV7; | TTGGCGAGTCCCTCTATGCGCGCTCGCTCGCT |
| No: 173 | Recogn. Site: | CGGTGGGGCCTGCGGACCAAAGGTCCGCAGA |
| | Nt.BstNBI; Format: | CGGCAGAGCTCTGCTCTGCCGGCCCCACCGAG |
| | t1 | CGAGCGAGCGCGCATAGAGGGACTCGCCAA |

TABLE 17
Reverse Complement of Nicking Enzyme Targets

| SEQ ID: | Name | Sequence |
|---|---|---|
| SEQ ID | wt_AAV1 | AACGGGTGAGGGAGAGACGCGCGAGCGAGCG |
| No: 186 | | AGCCACCCCGGACGCCTGGTTTCCAGGCGTCTG |
| | | CCGTCTCGAGACGAGACGGCCGGGGTGGCTCG |
| | | CTC |
| SEQ ID | AAV1_Nb.BbvCI_BL | AACGGGTGAGGGGGAGTCGCGCGAGCGAGCGA |
| No: 187 | | GCCACCCCGGACGCCTGGTTTCCAGGCGTCTGC |
| | | CGTCTCGAGACGAGACGGCCGGGGTGGCTCGC |
| | | TC |
| SEQ ID | AAV1_Nb.BbvCI_TL | AACGGGTGAGGGCGACTCCCGCGAGCGAGCGA |
| No: 188 | | GCCACCCCGGACGCCTGGTTTCCAGGCGTCTGC |
| | | CGTCTCGAGACGAGACGGCCGGGGGGCTCGC |
| | | TC |
| SEQ ID | AAV1_Nb.BsmI_BL | AACGGGTGAGGGACTTACGCGCGAGCGAGCGA |
| No: 189 | | GCCACCCCGGACGCCTGGTTTCCAGGCGTCTGC |
| | | CGTCTCGAGACGAGACGGCCGGGGTGGCTCGC |
| | | TC |
| SEQ ID | AAV1_Nb.BsmI_TL | AACGGGTGAGGGAGAGACGCGTAAGCGAGCG |
| No: 190 | | AGCCACCCCGGACGCCTGGTTTCCAGGCGTCTG |
| | | CCGTCTCGAGACGAGACGGCCGGGGTGGCTCG |
| | | CTC |
| SEQ ID | AAV1_Nb.BsrDI_BL | AACGGGTGAGGGCGTTACGCGCGAGCGAGCGA |
| No: 191 | | GCCACCCCGGACGCCTGGTTTCCAGGCGTCTGC |
| | | CGTCTCGAGACGAGACGGCCGGGGTGGCTCGC |
| | | TC |
| SEQ ID | AAV1_Nb.BsrDI_TL | AACGGGTGAGGGAGTAACGCGCGAGCGAGCGA |
| No: 192 | | GCCACCCCGGACGCCTGGTTTCCAGGCGTCTGC |
| | | CGTCTCGAGACGAGACGGCCGGGGTGGCTCGC |
| | | TC |
| SEQ ID | AAV1_Nb.BssSI_BL | AACGGGTGCTCGAGAGACGCGCGAGCGAGCGA |
| No: 193 | | GCCACCCCGGACGCCTGGTTTCCAGGCGTCTGC |
| | | CGTCTCGAGACGAGACGGCCGGGGTGGCTCGC |
| | | TC |
| SEQ ID | AAV1_Nb.BssSI_TL | AACGGGTGAGGGAGCACCGCGCGAGCGAGCGA |
| No: 194 | | GCCACCCCGGACGCCTGGTTTCCAGGCGTCTGC |
| | | CGTCTCGAGACGAGACGGCCGGGGTGGCTCGC |
| | | TC |
| SEQ ID | AAV1_Nb.BtsI_BL | AACGGGTGAGGGCGTCACGCGCGAGCGAGCGA |
| No: 195 | | GCCACCCCGGACGCCTGGTTTCCAGGCGTCTGC |
| | | CGTCTCGAGACGAGACGGCCGGGGTGGCTCGC |
| | | TC |
| SEQ ID | AAV1_Nb.BtsI_TL | AACGGGTGAGGGAGTGACGCGCGAGCGAGCGA |
| No: 196 | | GCCACCCCGGACGCCTGGTTTCCAGGCGTCTGC |
| | | CGTCTCGAGACGAGACGGCCGGGGTGGCTCGC |
| | | TC |
| SEQ ID | AAV1_Nt.AlwI_BL | AACGGGTGAGGGAGCTAGGCGCGAGCGAGCGA |
| No: 197 | | GCCACCCCGGACGCCTGGTTTCCAGGCGTCTGC |
| | | CGTCTCGAGACGAGACGGCCGGGGTGGCTCGC |
| | | TC |
| SEQ ID | AAV1_Nt.AlwI_BL | AACGGGTGACCTAGAGACGCGCGAGCGAGCGA |
| No: 198 | | GCCACCCCGGACGCCTGGTTTCCAGGCGTCTGC |
| | | CGTCTCGAGACGAGACGGCCGGGGTGGCTCGC |
| | | TC |
| SEQ ID | AAV1_Nt.BbvCI_TL | AACGGGTGAGGGCGACTCCCGCGAGCGAGCGA |
| No: 199 | | GCCACCCCGGACGCCTGGTTTCCAGGCGTCTGC |
| | | CGTCTCGAGACGAGACGGCCGGGGTGGCTCGC |
| | | TC |
| SEQ ID | AAV1_Nt.BbvCI_BL | AACGGGTGAGGGGGAGTCGCGCGAGCGAGCGA |
| No: 200 | | GCCACCCCGGACGCCTGGTTTCCAGGCGTCTGC |
| | | CGTCTCGAGACGAGACGGCCGGGGTGGCTCGC |
| | | TC |
| SEQ ID | AAV1_Nt.BsmAI_TL | AACGGGTGACTCTGAGACGCGCGAGCGAGCGA |
| No: 201 | | GCCACCCCGGACGCCTGGTTTCCAGGCGTCTGC |
| | | CGTCTCGAGACGAGACGGCCGGGGTGGCTCGC |
| | | TC |
| SEQ ID | AAV1_Nt.BsmAI_BL | AACGGGTGAGGCAGAGACGCGCGAGCGAGCG |
| No: 202 | | AGCCACCCCGGACGCCTGGTTTCCAGGCGTCTG |
| | | CCGTCTCGAGACGAGACGGCCGGGGTGGCTCG |
| | | CTC |
| SEQ ID | AAV1_Nt.BspQI_TL | AACGGGTGAGGGCTTCTCGCGCGAGCGAGCGA |
| No: 203 | | GCCACCCCGGACGCCTGGTTTCCAGGCGTCTGC |
| | | CGTCTCGAGACGAGACGGCCGGGGGGCTCGC |
| | | TC |
| SEQ ID | AAV1_Nt.BspQI_BL | AACGGGTGAGGGCGAGAAGCGCGAGCGAGCG |
| No: 204 | | AGCCACCCCGGACGCCTGGTTTCCAGGCGTCTG |
| | | CCGTCTCGAGACGAGACGGCCGGGGTGGCTCG |
| | | CTC |
| SEQ ID | AAV1_Nt.BstNBI_TL | AACGGGTGAGGGAGAGACGCGCTGAGCGAGCG |
| No: 205 | | AGCCACCCCGGACGCCTGGTTTCCAGGCGTCTG |
| | | CCGTCTCGAGACGAGACGGCCGGGGTGGCTCG |
| | | CTC |
| SEQ ID | AAV1_Nt.BstNBI_BL | AACGGCTCAGGGAGAGACGCGCGAGCGAGCGA |
| No: 206 | | GCCACCCCGGACGCCTGGTTTCCAGGCGTCTGC |
| | | CGTCTCGAGACGAGACGGCCGGGGGGCTCGC |
| | | TC |
| SEQ ID | wt_AAV2 | AACCGGTGAGGGAGAGACGCGCGAGCGAGCG |
| No: 207 | | AGTGACTCCGGCCCGCTGGTTTCCAGCGGGCTG |
| | | CGGGCCCGAAACGGGCCCGCCGGAGTCACTCG |
| | | CTC |
| SEQ ID | AAV2_Nb.BbvCI_BL | AACCGGTGAGGGGGAGTCGCGCGAGCGAGCGA |
| No: 208 | | GTGACTCCGGCCCGCTGGTTTCCAGCGGGCTGC |
| | | GGGCCCGAAACGGGCCCGCCGGAGTCACTCGC |
| | | TC |
| SEQ ID | AAV2_Nb.BbvCI_TL | AACCGGTGAGGGCGACTCCCGCGAGCGAGCGA |
| No: 209 | | GTGACTCCGGCCCGCTGGTTTCCAGCGGGCTGC |
| | | GGGCCCGAAACGGGCCCGCCGGAGTCACTCGC |
| | | TC |
| SEQ ID | AAV2_Nb.BsmI_BL | AACCGGTGAGGGACTTACGCGCGAGCGAGCGA |
| No: 210 | | GTGACTCCGGCCCGCTGGTTTCCAGCGGGCTGC |
| | | GGGCCCGAAACGGGCCCGCCGGAGTCACTCGC |
| | | TC |
| SEQ ID | AAV2_Nb.BsmI_TL | AACCGGTGAGGGAGAGACGCGTAAGCGAGCGA |
| No: 211 | | GTGACTCCGGCCCGCTGGTTTCCAGCGGGCTGC |
| | | GGGCCCGAAACGGGCCCGCCGGAGTCACTCGC |
| | | TC |
| SEQ ID | AAV2_Nb.BsrDI_BL | AACCGGTGAGGGCGTTACGCGCGAGCGAGCGA |
| No: 212 | | GTGACTCCGGCCCGCTGGTTTCCAGCGGGCTGC |
| | | GGGCCCGAAACGGGCCCGCCGGAGTCACTCGC |
| | | TC |
| SEQ ID | AAV2_Nb.BsrDI_TL | AACCGGTGAGGGAGTAACGCGCGAGCGAGCGA |
| No: 213 | | GTGACTCCGGCCCGCTGGTTTCCAGCGGGCTGC |
| | | GGGCCCGAAACGGGCCCGCCGGAGTCACTCGC |
| | | TC |
| SEQ ID | AAV2_Nb.BssSI_BL | AACCGGTGCTCGAGAGACGCGCGAGCGAGCGA |
| No: 214 | | GTGACTCCGGCCCGCTGGTTTCCAGCGGGCTGC |
| | | GGGCCCGAAACGGGCCCGCCGGAGTCACTCGC |
| | | TC |
| SEQ ID | AAV2_Nb.BssSI_TL | AACCGGTGAGGGAGCACCGCGCGAGCGAGCGA |
| No: 215 | | GTGACTCCGGCCCGCTGGTTTCCAGCGGGCTGC |
| | | GGGCCCGAAACGGGCCCGCCGGAGTCACTCGC |
| | | TC |
| SEQ ID | AAV2_Nb.BtsI_BL | AACCGGTGAGGGCGTCACGCGCGAGCGAGCGA |
| No: 216 | | GTGACTCCGGCCCGCTGGTTTCCAGCGGGCTGC |
| | | GGGCCCGAAACGGGCCCGCCGGAGTCACTCGC |
| | | TC |
| SEQ ID | AAV2_Nb.BtsI_TL | AACCGGTGAGGGAGTGACGCGCGAGCGAGCGA |
| No: 217 | | GTGACTCCGGCCCGCTGGTTTCCAGCGGGCTGC |
| | | GGGCCCGAAACGGGCCCGCCGGAGTCACTCGC |
| | | TC |
| SEQ ID | AAV2_Nt.AlwI_BL | AACCGGTGAGGGAGCTAGGCGCGAGCGAGCGA |
| No: 218 | | GTGACTCCGGCCCGCTGGTTTCCAGCGGGCTGC |
| | | GGGCCCGAAACGGGCCCGCCGGAGTCACTCGC |
| | | TC |
| SEQ ID | AAV2_Nt.AlwI_BL | AACCGGTGACCTAGAGACGCGCGAGCGAGCGA |
| No: 219 | | GTGACTCCGGCCCGCTGGTTTCCAGCGGGCTGC |
| | | GGGCCCGAAACGGGCCCGCCGGAGTCACTCGC |
| | | TC |
| SEQ ID | AAV2_Nt.BbvCI_TL | AACCGGTGAGGGCGACTCCCGCGAGCGAGCGA |
| No: 220 | | GTGACTCCGGCCCGCTGGTTTCCAGCGGGCTGC |
| | | GGGCCCGAAACGGGCCCGCCGGAGTCACTCGC |
| | | TC |
| SEQ ID | AAV2_Nt.BbvCI_BL | AACCGGTGAGGGGGAGTCGCGCGAGCGAGCGA |
| No: 221 | | GTGACTCCGGCCCGCTGGTTTCCAGCGGGCTGC |
| | | GGGCCCGAAACGGGCCCGCCGGAGTCACTCGC |
| | | TC |
| SEQ ID | AAV2_Nt.BsmAI_TL | AACCGGTGACTCTGAGACGCGCGAGCGAGCGA |
| No: 222 | | GTGACTCCGGCCCGCTGGTTTCCAGCGGGCTGC |
| | | GGGCCCGAAACGGGCCCGCCGGAGTCACTCGC |
| | | TC |
| SEQ ID | AAV2_Nt.BsmAI_BL | AACCGGTGAGGCAGAGACGCGCGAGCGAGCGA |
| No: 223 | | GTGACTCCGGCCCGCTGGTTTCCAGCGGGCTGC |
| | | GGGCCCGAAACGGGCCCGCCGGAGTCACTCGC |
| | | TC |
| SEQ ID | AAV2_Nt.BspQI_TL | AACCGGTGAGGGCTTCTCGCGCGAGCGAGCGA |
| No: 224 | | GTGACTCCGGCCCGCTGGTTTCCAGCGGGCTGC |
| | | GGGCCCGAAACGGGCCCGCCGGAGTCACTCGC |
| | | TC |
| SEQ ID | AAV2_Nt.BspQI_BL | AACCGGTGAGGGCGAGAAGCGCGAGCGAGCG |
| No: 225 | | AGTGACTCCGGCCCGCTGGTTTCCAGCGGGCTG |
| | | CGGGCCCGAAACGGGCCCGCCGGAGTCACTCG |
| | | CTC |
| SEQ ID | AAV2_Nt.BstNBI_TL | AACCGGTGAGGGAGAGACGCGCTGAGCGAGCG |
| No: 226 | | AGTGACTCCGGCCCGCTGGTTTCCAGCGGGCTG |
| | | CGGGCCCGAAACGGGCCCGCCGGAGTCACTCG |
| | | CTC |
| SEQ ID | AAV2_Nt.BstNBI_TL | AACCGCTCAGGGAGAGACGCGCGAGCGAGCGA |
| No: 227 | | GTGACTCCGGCCCGCTGGTTTCCAGCGGGCTGC |
| | | GGGCCCGAAACGGGCCCGCCGGAGTCACTCGC |
| | | TC |
| SEQ ID | wt_AAV3 | AACCGGTGAGGGAGATACGCGTGAGCGAGCGA |
| No: 228 | | GCCACCCCGGACCGCTGGTTTCCAGCGGTCTGC |
| | | CTGCACGAAACGTGCAGGCCGGGGTGGCTCGC |
| | | TC |
| SEQ ID | AAV3_Nb.BbvCI_BL | AACCGGTGAGGGGGAGTCGCGTGAGCGAGCGA |
| No: 229 | | GCCACCCCGGACCGCTGGTTTCCAGCGGTCTGC |
| | | CTGCACGAAACGTGCAGGCCGGGGTGGCTCGC |
| | | TC |
| SEQ ID | AAV3_Nb.BbvCI_TL | AACCGGTGAGGGCGACTCCCGTGAGCGAGCGA |
| No: 230 | | GCCACCCCGGACCGCTGGTTTCCAGCGGTCTGC |
| | | CTGCACGAAACGTGCAGGCCGGGGTGGCTCGC |
| | | TC |
| SEQ ID | AAV3_Nb.BsmI_BL | AACCGGTGAGGGACTTACGCGTGAGCGAGCGA |
| No: 231 | | GCCACCCCGGACCGCTGGTTTCCAGCGGTCTGC |
| | | CTGCACGAAACGTGCAGGCCGGGGTGGCTCGC |
| | | TC |
| SEQ ID | AAV3_Nb.BsmI_TL | AACCGGTGAGGGAGATACGCGTAAGCGAGCGA |
| No: 232 | | GCCACCCCGGACCGCTGGTTTCCAGCGGTCTGC |
| | | CTGCACGAAACGTGCAGGCCGGGGTGGCTCGC |
| | | TC |
| SEQ ID | AAV3_Nb.BsrDI_BL | AACCGGTGAGGGCGTTACGCGTGAGCGAGCGA |
| No: 233 | | GCCACCCCGGACCGCTGGTTTCCAGCGGTCTGC |
| | | CTGCACGAAACGTGCAGGCCGGGGTGGCTCGC |
| | | TC |
| SEQ ID | AAV3_Nb.BsrDI_TL | AACCGGTGAGGGAGTAACGCGTGAGCGAGCGA |
| No: 234 | | GCCACCCCGGACCGCTGGTTTCCAGCGGTCTGC |
| | | CTGCACGAAACGTGCAGGCCGGGGGGCTCGC |
| | | TC |
| SEQ ID | AAV3_Nb.BssSI_BL | AACCGGTGCTCGAGATACGCGTGAGCGAGCGA |
| No: 235 | | GCCACCCCGGACCGCTGGTTTCCAGCGGTCTGC |
| | | CTGCACGAAACGTGCAGGCCGGGGTGGCTCGC |
| | | TC |
| SEQ ID | AAV3_Nb.BssSI_TL | AACCGGTGAGGGAGCACCGCGTGAGCGAGCGA |
| No: 236 | | GCCACCCCGGACCGCTGGTTTCCAGCGGTCTGC |
| | | CTGCACGAAACGTGCAGGCCGGGGTGGCTCGC |
| | | TC |
| SEQ ID | AAV3_Nb.BtsI_BL | AACCGGTGAGGGCGTCACGCGTGAGCGAGCGA |
| No: 237 | | GCCACCCCGGACCGCTGGTTTCCAGCGGTCTGC |
| | | CTGCACGAAACGTGCAGGCCGGGGTGGCTCGC |
| | | TC |
| SEQ ID | AAV3_Nb.BtsI_TL | AACCGGTGAGGGAGTGACGCGTGAGCGAGCGA |
| No: 238 | | GCCACCCCGGACCGCTGGTTTCCAGCGGTCTGC |
| | | CTGCACGAAACGTGCAGGCCGGGGTGGCTCGC |
| | | TC |
| SEQ ID | AAV3_Nt.AlwI_BL | AACCGGTGAGGGAGCTAGGCGTGAGCGAGCGA |
| No: 239 | | GCCACCCCGGACCGCTGGTTTCCAGCGGTCTGC |
| | | CTGCACGAAACGTGCAGGCCGGGGTGGCTCGC |
| | | TC |
| SEQ ID | AAV3_Nt.AlwI_BL | AACCGGTGACCTAGATACGCGTGAGCGAGCGA |
| No: 240 | | GCCACCCCGGACCGCTGGTTTCCAGCGGTCTGC |
| | | CTGCACGAAACGTGCAGGCCGGGGTGGCTCGC |
| | | TC |
| SEQ ID | AAV3_Nt.BbvCI_TL | AACCGGTGAGGGCGACTCCCGTGAGCGAGCGA |
| No: 241 | | GCCACCCCGGACCGCTGGTTTCCAGCGGTCTGC |
| | | CTGCACGAAACGTGCAGGCCGGGGTGGCTCGC |
| | | TC |
| SEQ ID | AAV3_Nt.BbvCI_BL | AACCGGTGAGGGGGAGTCGCGTGAGCGAGCGA |
| No: 242 | | GCCACCCCGGACCGCTGGTTTCCAGCGGTCTGC |
| | | CTGCACGAAACGTGCAGGCCGGGGTGGCTCGC |
| | | TC |
| SEQ ID | AAV3_Nt.BsmAI_TL | AACCGGTGACTCTGATACGCGTGAGCGAGCGA |
| No: 243 | | GCCACCCCGGACCGCTGGTTTCCAGCGGTCTGC |
| | | CTGCACGAAACGTGCAGGCCGGGGTGGCTCGC |
| | | TC |
| SEQ ID | AAV3_Nt.BsmAI_BL | AACCGGTGAGGCAGAGACGCGTGAGCGAGCGA |
| No: 244 | | GCCACCCCGGACCGCTGGTTTCCAGCGGTCTGC |
| | | CTGCACGAAACGTGCAGGCCGGGGTGGCTCGC |
| | | TC |
| SEQ ID | AAV3_Nt.BspQI_TL | AACCGGTGAGGGCTTCTCGCGTGAGCGAGCGA |
| No: 245 | | GCCACCCCGGACCGCTGGTTTCCAGCGGTCTGC |
| | | CTGCACGAAACGTGCAGGCCGGGGTGGCTCGC |
| | | TC |
| SEQ ID | AAV3_Nt.BspQI_BL | AACCGGTGAGGGCGAGAAGCGTGAGCGAGCGA |
| No: 246 | | GCCACCCCGGACCGCTGGTTTCCAGCGGTCTGC |
| | | CTGCACGAAACGTGCAGGCCGGGGTGGCTCGC |
| | | TC |
| SEQ ID | AAV3_Nt.BstNBI_TL | AACCGGTGAGGGAGATACGCGCTGAGCGAGCG |
| No: 247 | | AGCCACCCCGGACCGCTGGTTTCCAGCGGTCTG |
| | | CCTGCACGAAACGTGCAGGCCGGGGTGGCTCG |
| | | CTC |
| SEQ ID | AAV3_Nt.BstNBI_BL | AACCGCTCAGGGAGATACGCGTGAGCGAGCGA |
| No: 248 | | GCCACCCCGGACCGCTGGTTTCCAGCGGTCTGC |
| | | CTGCACGAAACGTGCAGGCCGGGGTGGCTCGC |
| | | TC |
| SEQ ID | wt_AAV4_left | AACCGGTGAGGGAGATACGCGCGAGCGAGTGA |
| No: 249 | | GTGAGCCGGGACCTCTGGTTTCCAGAGGTCTGA |
| | | CGGCCGGAGACCGGCCGTCCCGGCTCACTCACT |
| | | CGCTCGCGCGTATCTCCCTCACCGGTT |
| SEQ ID | AAV4_left_Nb.BbvCI_BL | AACCGGTGAGGGGGAGTCGCGCGAGCGAGTGA |
| No: 250 | | GTGAGCCGGGACCTCTGGTTTCCAGAGGTCTGA |
| | | CGGCCGGAGACCGGCCGTCCCGGCTCACTCACT |
| | | CGCTCGCGCGACTCCCCCTCACCGGTT |
| SEQ ID | AAV4_left_Nb.BbvCI_TL | AACCGGTGAGGGCGACTCCCGCGAGCGAGTGA |
| No: 251 | | GTGAGCCGGGACCTCTGGTTTCCAGAGGTCTGA |
| | | CGGCCGGAGACCGGCCGTCCCGGCTCACTCACT |
| | | CGCTCGCGGGAGTCGCCCTCACCGGTT |
| SEQ ID | AAV4_left_Nb.BsmI_BL | AACCGGTGAGGGACTTACGCGCGAGCGAGTGA |
| No: 252 | | GTGAGCCGGGACCTCTGGTTTCCAGAGGTCTGA |
| | | CGGCCGGAGACCGGCCGTCCCGGCTCACTCACT |
| | | CGCTCGCGCGTAAGTCCCTCACCGGTT |
| SEQ ID | AAV4_left_Nb.BsmI_TL | AACCGGTGAGGGAGATACGCGTAAGCGAGTGA |
| No: 253 | | GTGAGCCGGGACCTCTGGTTTCCAGAGGTCTGA |
| | | CGGCCGGAGACCGGCCGTCCCGGCTCACTCACT |
| | | CGCTTACGCGTATCTCCCTCACCGGTT |
| SEQ ID | AAV4_left_Nb.BsrDI_BL | AACCGGTGAGGGCGTTACGCGCGAGCGAGTGA |
| No: 254 | | GTGAGCCGGGACCTCTGGTTTCCAGAGGTCTGA |
| | | CGGCCGGAGACCGGCCGTCCCGGCTCACTCACT |
| | | CGCTCGCGCGTAACGCCCTCACCGGTT |
| SEQ ID | AAV4_left_Nb.BsrDI_BL | AACCGGTGAGGGAGTAACGCGCGAGCGAGTGA |
| No: 255 | | GTGAGCCGGGACCTCTGGTTTCCAGAGGTCTGA |
| | | CGGCCGGAGACCGGCCGTCCCGGCTCACTCACT |
| | | CGCTCGCGCGTTACTCCCTCACCGGTT |
| SEQ ID | AAV4_left_Nb.BssSI_BL | AACCGGTGCTCGAGATACGCGCGAGCGAGTGA |
| No: 256 | | GTGAGCCGGGACCTCTGGTTTCCAGAGGTCTGA |
| | | CGGCCGGAGACCGGCCGTCCCGGCTCACTCACT |
| | | CGCTCGCGCGTATCTCGAGCACCGGTT |
| SEQ ID | AAV4_left_Nb.BssSI_TL | AACCGGTGAGGGAGCACCGCGCGAGCGAGTGA |
| No: 257 | | GTGAGCCGGGACCTCTGGTTTCCAGAGGTCTGA |
| | | CGGCCGGAGACCGGCCGTCCCGGCTCACTCACT |
| | | CGCTCGCGCGGTGCTCCCTCACCGGTT |
| SEQ ID | AAV4_left_Nb.BtsI_BL | AACCGGTGAGGGCGTCACGCGCGAGCGAGTGA |
| No: 258 | | GTGAGCCGGGACCTCTGGTTTCCAGAGGTCTGA |
| | | CGGCCGGAGACCGGCCGTCCCGGCTCACTCACT |
| | | CGCTCGCGCGTGACGCCCTCACCGGTT |
| SEQ ID | AAV4_left_Nb.BtsI_TL | AACCGGTGAGGGAGTGACGCGCGAGCGAGTGA |
| No: 259 | | GTGAGCCGGGACCTCTGGTTTCCAGAGGTCTGA |
| | | CGGCCGGAGACCGGCCGTCCCGGCTCACTCACT |
| | | CGCTCGCGCGTCACTCCCTCACCGGTT |
| SEQ ID | AAV4_left_Nt.AlwI_BL | AACCGGTGAGGGAGCTAGGCGCGAGCGAGTGA |
| No: 260 | | GTGAGCCGGGACCTCTGGTTTCCAGAGGTCTGA |
| | | CGGCCGGAGACCGGCCGTCCCGGCTCACTCACT |
| | | CGCTCGCGCCTAGCTCCCTCACCGGTT |
| SEQ ID | AAV4_left_Nt.AlwI_BL | AACCGGTGACCTAGATACGCGCGAGCGAGTGA |
| No: 261 | | GTGAGCCGGGACCTCTGGTTTCCAGAGGTCTGA |
| | | CGGCCGGAGACCGGCCGTCCCGGCTCACTCACT |
| | | CGCTCGCGCGTATCTAGGTCACCGGTT |
| SEQ ID | AAV4_left_Nt.BbvCI_TL | AACCGGTGAGGGCGACTCCCGCGAGCGAGTGA |
| No: 262 | | GTGAGCCGGGACCTCTGGTTTCCAGAGGTCTGA |
| | | CGGCCGGAGACCGGCCGTCCCGGCTCACTCACT |
| | | CGCTCGCGGGAGTCGCCCTCACCGGTT |
| SEQ ID | AAV4_left_Nt.BbvCI_BL | AACCGGTGAGGGGGAGTCGCGCGAGCGAGTGA |
| No: 263 | | GTGAGCCGGGACCTCTGGTTTCCAGAGGTCTGA |
| | | CGGCCGGAGACCGGCCGTCCCGGCTCACTCACT |
| | | CGCTCGCGCGACTCCCCCTCACCGGTT |
| SEQ ID | AAV4_left_Nt.BsmAI_TL | AACCGGTGACTCTGATACGCGCGAGCGAGTGA |
| No: 264 | | GTGAGCCGGGACCTCTGGTTTCCAGAGGTCTGA |
| | | CGGCCGGAGACCGGCCGTCCCGGCTCACTCACT |
| | | CGCTCGCGCGTATCAGAGTCACCGGTT |
| SEQ ID | AAV4_left_Nt.BsmAI_BL | AACCGGTGAGGCAGAGACGCGCGAGCGAGTGA |
| No: 265 | | GTGAGCCGGGACCTCTGGTTTCCAGAGGTCTGA |
| | | CGGCCGGAGACCGGCCGTCCCGGCTCACTCACT |
| | | CGCTCGCGCGTCTCTGCCTCACCGGTT |
| SEQ ID | AAV4_left_Nt.BspQI_TL | AACCGGTGAGGGCTTCTCGCGCGAGCGAGTGA |
| No: 266 | | GTGAGCCGGGACCTCTGGTTTCCAGAGGTCTGA |
| | | CGGCCGGAGACCGGCCGTCCCGGCTCACTCACT |
| | | CGCTCGCGCGAGAAGCCCTCACCGGTT |
| SEQ ID | AAV4_left_Nt.BspQI_BL | AACCGGTGAGGGCGAGAAGCGCGAGCGAGTGA |
| No: 267 | | GTGAGCCGGGACCTCTGGTTTCCAGAGGTCTGA |
| | | CGGCCGGAGACCGGCCGTCCCGGCTCACTCACT |
| | | CGCTCGCGCTTCTCGCCCTCACCGGTT |
| SEQ ID | AAV4_left_Nt.BstNBI_TL | AACCGGTGAGGGAGATACGCGCTGAGCGAGTG |
| No: 268 | | AGTGAGCCGGGACCTCTGGTTTCCAGAGGTCTG |
| | | ACGGCCGGAGACCGGCCGTCCCGGCTCACTCA |
| | | CTCGCTCAGCGCGTATCTCCCTCACCGGTT |
| SEQ ID | AAV4_left_Nt.BstNBI_BL | AACCGCTCAGGGAGATACGCGCGAGCGAGTGA |
| No: 269 | | GTGAGCCGGGACCTCTGGTTTCCAGAGGTCTGA |
| | | CGGCCGGAGACCGGCCGTCCCGGCTCACTCACT |
| | | CGCTCGCGCGTATCTCCCTGAGCGGTT |
| SEQ ID | wt_AAV4_Right | AACCGGTGTAATCGATACGCGCGAGCGAGTGA |
| No: 270 | | GTGAGCCGGGACCTCTGGTTTCCAGAGGTCTGA |
| | | CGGCCGGAGACCGGCCGTCCCGGCTCACTCACT |
| | | CGCTCGCGCGTATCTCCCTCACCGGTT |
| SEQ ID | AAV4_Right_Nb.BbvCI_BL | AACCGGTGTAATGGAGTCGCGCGAGCGAGTGA |
| No: 271 | | GTGAGCCGGGACCTCTGGTTTCCAGAGGTCTGA |
| | | CGGCCGGAGACCGGCCGTCCCGGCTCACTCACT |
| | | CGCTCGCGCGACTCCCCCTCACCGGTT |
| SEQ ID | AAV4_Right_Nb.BbvCI_TL | AACCGGTGTAATCGACTCCCGCGAGCGAGTGA |
| No: 272 | | GTGAGCCGGGACCTCTGGTTTCCAGAGGTCTGA |
| | | CGGCCGGAGACCGGCCGTCCCGGCTCACTCACT |
| | | CGCTCGCGGGAGTCGCCCTCACCGGTT |
| SEQ ID | AAV4_Right_Nb.BsmI_BL | AACCGGTGTAATCCTTACGCGCGAGCGAGTGA |
| No: 273 | | GTGAGCCGGGACCTCTGGTTTCCAGAGGTCTGA |
| | | CGGCCGGAGACCGGCCGTCCCGGCTCACTCACT |
| | | CGCTCGCGCGTAAGTCCCTCACCGGTT |
| SEQ ID | AAV4_Right_Nb.BsmI_TL | AACCGGTGTAATCGATACGCGTAAGCGAGTGA |
| No: 274 | | GTGAGCCGGGACCTCTGGTTTCCAGAGGTCTGA |
| | | CGGCCGGAGACCGGCCGTCCCGGCTCACTCACT |
| | | CGCTTACGCGTATCTCCCTCACCGGTT |
| SEQ ID | AAV4_Right_Nb.BsrDI_BL | AACCGGTGTAATCGTTACGCGCGAGCGAGTGA |
| No: 275 | | GTGAGCCGGGACCTCTGGTTTCCAGAGGTCTGA |
| | | CGGCCGGAGACCGGCCGTCCCGGCTCACTCACT |
| | | CGCTCGCGCGTAACGCCCTCACCGGTT |
| SEQ ID | AAV4_Right_Nb.BsrDI_TL | AACCGGTGTAATCGTAACGCGCGAGCGAGTGA |
| No: 276 | | GTGAGCCGGGACCTCTGGTTTCCAGAGGTCTGA |
| | | CGGCCGGAGACCGGCCGTCCCGGCTCACTCACT |
| | | CGCTCGCGCGTTACTCCCTCACCGGTT |
| SEQ ID | AAV4_Right_Nb.BsSSI_BL | AACCGGTGCTCTCGATACGCGCGAGCGAGTGA |
| No: 277 | | GTGAGCCGGGACCTCTGGTTTCCAGAGGTCTGA |
| | | CGGCCGGAGACCGGCCGTCCCGGCTCACTCACT |
| | | CGCTCGCGCGTATCTCGAGCACCGGTT |
| SEQ ID | AAV4_Right_Nb.BsSSI_TL | AACCGGTGTAAGAGCACCGCGCGAGCGAGTGA |
| No: 278 | | GTGAGCCGGGACCTCTGGTTTCCAGAGGTCTGA |
| | | CGGCCGGAGACCGGCCGTCCCGGCTCACTCACT |
| | | CGCTCGCGCGGTGCTCCCTCACCGGTT |
| SEQ ID | AAV4_Right_Nb.BtSI_BL | AACCGGTGTAATCGTCACGCGCGAGCGAGTGA |
| No: 279 | | GTGAGCCGGGACCTCTGGTTTCCAGAGGTCTGA |
| | | CGGCCGGAGACCGGCCGTCCCGGCTCACTCACT |
| | | CGCTCGCGCGTGACGCCCTCACCGGTT |
| SEQ ID | AAV4_Right_Nb.BtSI_TL | AACCGGTGTAATCGTGACGCGCGAGCGAGTGA |
| No: 280 | | GTGAGCCGGGACCTCTGGTTTCCAGAGGTCTGA |
| | | CGGCCGGAGACCGGCCGTCCCGGCTCACTCACT |
| | | CGCTCGCGCGTCACTCCCTCACCGGTT |
| SEQ ID | AAV4_Right_Nt.AlwI_BL | AACCGGTGTAATCGCTAGGCGCGAGCGAGTGA |
| No: 281 | | GTGAGCCGGGACCTCTGGTTTCCAGAGGTCTGA |
| | | CGGCCGGAGACCGGCCGTCCCGGCTCACTCACT |
| | | CGCTCGCGCCTAGCTCCCTCACCGGTT |
| SEQ ID | AAV4_Right_Nt.AlwI_BL | AACCGGTGTCCTAGATACGCGCGAGCGAGTGA |
| No: 282 | | GTGAGCCGGGACCTCTGGTTTCCAGAGGTCTGA |
| | | CGGCCGGAGACCGGCCGTCCCGGCTCACTCACT |
| | | CGCTCGCGCGTATCTAGGTCACCGGTT |
| SEQ ID | AAV4_Right_Nt.BbvCI_TL | AACCGGTGTAATCGACTCCCGCGAGCGAGTGA |
| No: 283 | | GTGAGCCGGGACCTCTGGTTTCCAGAGGTCTGA |
| | | CGGCCGGAGACCGGCCGTCCCGGCTCACTCACT |
| | | CGCTCGCGGGAGTCGCCCTCACCGGTT |
| SEQ ID | AAV4_Right_Nt.BbvCI_BL | AACCGGTGTAATGGAGTCGCGCGAGCGAGTGA |
| No: 284 | | GTGAGCCGGGACCTCTGGTTTCCAGAGGTCTGA |
| | | CGGCCGGAGACCGGCCGTCCCGGCTCACTCACT |
| | | CGCTCGCGCGACTCCCCCTCACCGGTT |
| SEQ ID | AAV4_Right_Nt.BsmAI_TL | AACCGGTGTCTCTGATACGCGCGAGCGAGTGA |
| No: 285 | | GTGAGCCGGGACCTCTGGTTTCCAGAGGTCTGA |
| | | CGGCCGGAGACCGGCCGTCCCGGCTCACTCACT |
| | | CGCTCGCGCGTATCAGAGTCACCGGTT |
| SEQ ID | AAV4_Right_Nt.BsmAI_BL | AACCGGTGTAACAGAGACGCGCGAGCGAGTGA |
| No: 286 | | GTGAGCCGGGACCTCTGGTTTCCAGAGGTCTGA |
| | | CGGCCGGAGACCGGCCGTCCCGGCTCACTCACT |
| | | CGCTCGCGCGTCTCTGCCTCACCGGTT |
| SEQ ID | AAV4_Right_Nt.BspQI_TL | AACCGGTGTAATCTTCTCGCGCGAGCGAGTGA |
| No: 287 | | GTGAGCCGGGACCTCTGGTTTCCAGAGGTCTGA |
| | | CGGCCGGAGACCGGCCGTCCCGGCTCACTCACT |
| | | CGCTCGCGCGAGAAGCCCTCACCGGTT |
| SEQ ID | AAV4_Right_Nt.BspQI_BL | AACCGGTGTAATCGAGAAGCGCGAGCGAGTGA |
| No: 288 | | GTGAGCCGGGACCTCTGGTTTCCAGAGGTCTGA |
| | | CGGCCGGAGACCGGCCGTCCCGGCTCACTCACT |
| | | CGCTCGCGCTTCTCGCCCTCACCGGTT |
| SEQ ID | AAV4_Right_Nt.BstNBI_TL | AACCGGTGTAATCGATACGCGCTGAGCGAGTG |
| No: 289 | | AGTGAGCCGGGACCTCTGGTTTCCAGAGGTCTG |
| | | ACGGCCGGAGACCGGCCGTCCCGGCTCACTCA |
| | | CTCGCTCAGCGCGTATCTCCCTCACCGGTT |
| SEQ ID | AAV4_Right_Nt.BstNBI_BL | AACCGGTCTCAGCGATACGCGCGAGCGAGTGA |
| No: 290 | | GTGAGCCGGGACCTCTGGTTTCCAGAGGTCTGA |
| | | CGGCCGGAGACCGGCCGTCCCGGCTCACTCACT |
| | | CGCTCGCGCGTATCTCTGAGACCGGTT |
| SEQ ID | wt_AAV5 | GAGAGGGGGGACAGCGCAAGCGAGCGAGCGA |
| No: 291 | | CCGAGCAAACCCCCCCACCGTCGAGTTTCTCGA |
| | | CGGTCTGCTGCCGGGAGACCGGCAGCGGGGGG |
| | | GTTTGCTCGGTCGCTCGCTCGCTTGCGCTGTCC |
| | | CCCCTCTC |
| SEQ ID | AAV5_Nb.BbvCI_BL | GAGAGGGGAGTCGGCGCAAGCGAGCGAGCGA |
| No: 292 | | CCGAGCAAACCCCCCCACCGTCGAGTTTCTCGA |
| | | CGGTCTGCTGCCGGGAGACCGGCAGCGGGGGG |
| | | GTTTGCTCGGTCGCTCGCTCGCTTGCGCCGACT |
| | | CCCCTCTC |
| SEQ ID | AAV5_Nb.BbvCI_TL | GAGAGGGGCGACTCCGCAAGCGAGCGAGCGAC |
| No: 293 | | CGAGCAAACCCCCCCACCGTCGAGTTTCTCGAC |
| | | GGTCTGCTGCCGGGAGACCGGCAGCGGGGGGG |
| | | TTTGCTCGGTCGCTCGCTCGCTTGCGGAGTCGC |
| | | CCCTCTC |
| SEQ ID | AAV5_Nb.BsmI_BL | GAGAGGGGCTTACGCGCAAGCGAGCGAGCGAC |
| No: 294 | | CGAGCAAACCCCCCCACCGTCGAGTTTCTCGAC |
| | | GGTCTGCTGCCGGGAGACCGGCAGCGGGGGGG |
| | | TTTGCTCGGTCGCTCGCTCGCTTGCGCGTAAGC |
| | | CCCTCTC |
| SEQ ID | AAV5_Nb.BsmI_TL | GAGAGGGGGGACAGCGTAAGCGAGCGAGCGA |
| No: 295 | | CCGAGCAAACCCCCCCACCGTCGAGTTTCTCGA |
| | | CGGTCTGCTGCCGGGAGACCGGCAGCGGGGGG |
| | | GTTTGCTCGGTCGCTCGCTCGCTTACGCTGTCC |
| | | CCCCTCTC |
| SEQ ID | AAV5_Nb.BsrDI_BL | GAGAGGCGTTACAGCGCAAGCGAGCGAGCGAC |
| No: 296 | | CGAGCAAACCCCCCCACCGTCGAGTTTCTCGAC |
| | | GGTCTGCTGCCGGGAGACCGGCAGCGGGGGGG |
| | | TTTGCTCGGTCGCTCGCTCGCTTGCGCTGTAAC |
| | | GCCTCTC |
| SEQ ID | AAV5_Nb.BsrDI_TL | GAGAGGGGGTAACGCGCAAGCGAGCGAGCGA |
| No: 297 | | CCGAGCAAACCCCCCCACCGTCGAGTTTCTCGA |
| | | CGGTCTGCTGCCGGGAGACCGGCAGCGGGGGG |
| | | GTTTGCTCGGTCGCTCGCTCGCTTGCGCGTTAC |
| | | CCCCTCTC |
| SEQ ID | AAV5_Nb.BssSI_BL | GAGTGCTCGGACAGCGCAAGCGAGCGAGCGAC |
| No: 298 | | CGAGCAAACCCCCCCACCGTCGAGTTTCTCGAC |
| | | GGTCTGCTGCCGGGAGACCGGCAGCGGGGGGG |
| | | TTTGCTCGGTCGCTCGCTCGCTTGCGCTGTCCG |
| | | AGCACTC |
| SEQ ID | AAV5_Nb.BssSI_TL | GAGAGGGAGCACAGCGCAAGCGAGCGAGCGA |
| No: 299 | | CCGAGCAAACCCCCCCACCGTCGAGTTTCTCGA |
| | | CGGTCTGCTGCCGGGAGACCGGCAGCGGGGGG |
| | | GTTTGCTCGGTCGCTCGCTCGCTTGCGCTGTGC |
| | | TCCCTCTC |
| SEQ ID | AAV5_Nb.BtsI_BL | GAGAGGCGTCACAGCGCAAGCGAGCGAGCGAC |
| No: 300 | | CGAGCAAACCCCCCCACCGTCGAGTTTCTCGAC |
| | | GGTCTGCTGCCGGGAGACCGGCAGCGGGGGGG |
| | | TTTGCTCGGTCGCTCGCTCGCTTGCGCTGTGAC |
| | | GCCTCTC |
| SEQ ID | AAV5_Nb.BtsI_TL | GAGAGGGGTGACGGCGCAAGCGAGCGAGCGA |
| No: 301 | | CCGAGCAAACCCCCCCACCGTCGAGTTTCTCGA |
| | | CGGTCTGCTGCCGGGAGACCGGCAGCGGGGGG |
| | | GTTTGCTCGGTCGCTCGCTCGCTTGCGCCGTCA |
| | | CCCCTCTC |
| SEQ ID | AAV5_Nt.AlwI_BL | GAGAGGGGGGACAGCGCTAGGGAGCGAGCGA |
| No: 302 | | CCGAGCAAACCCCCCCACCGTCGAGTTTCTCGA |
| | | CGGTCTGCTGCCGGGAGACCGGCAGCGGGGGG |
| | | GTTTGCTCGGTCGCTCGCTCCCTAGCGCTGTCC |
| | | CCCCTCTC |
| SEQ ID | AAV5_Nt.AlwI_BL | GAGAGGGGGCCTAGCGCAAGCGAGCGAGCGAC |
| No: 303 | | CGAGCAAACCCCCCCACCGTCGAGTTTCTCGAC |
| | | GGTCTGCTGCCGGGAGACCGGCAGCGGGGGGG |
| | | TTTGCTCGGTCGCTCGCTCGCTTGCGCTAGGCC |
| | | CCCTCTC |
| SEQ ID | AAV5_Nt.BbvCI_TL | GAGAGGGGCGACTCCGCAAGCGAGCGAGCGAC |
| No: 304 | | CGAGCAAACCCCCCCACCGTCGAGTTTCTCGAC |
| | | GGTCTGCTGCCGGGAGACCGGCAGCGGGGGGG |
| | | TTTGCTCGGTCGCTCGCTCGCTTGCGGAGTCGC |
| | | CCCTCTC |
| SEQ ID | AAV5_Nt.BbvCI_BL | GAGAGGGGAGTCGGCGCAAGCGAGCGAGCGA |
| No: 305 | | CCGAGCAAACCCCCCCACCGTCGAGTTTCTCGA |
| | | CGGTCTGCTGCCGGGAGACCGGCAGCGGGGGG |
| | | GTTTGCTCGGTCGCTCGCTCGCTTGCGCCGACT |
| | | CCCCTCTC |
| SEQ ID | AAV5_Nt.BsmAI_TL | GAGAGGGGGCTCTGCGCAAGCGAGCGAGCGAC |
| No: 306 | | CGAGCAAACCCCCCCACCGTCGAGTTTCTCGAC |
| | | GGTCTGCTGCCGGGAGACCGGCAGCGGGGGGG |
| | | TTTGCTCGGTCGCTCGCTCGCTTGCGCAGAGCC |
| | | CCCTCTC |
| SEQ ID | AAV5_Nt.BsmAI_BL | GAGAGGGGGCAGAGCGCAAGCGAGCGAGCGA |
| No: 307 | | CCGAGCAAACCCCCCCACCGTCGAGTTTCTCGA |
| | | CGGTCTGCTGCCGGGAGACCGGCAGCGGGGGG |
| | | GTTTGCTCGGTCGCTCGCTCGCTTGCGCTCTGC |
| | | CCCCTCTC |
| SEQ ID | AAV5_Nt.BspQI_TL | GAGAGGGCTTCTCGCGCAAGCGAGCGAGCGAC |
| No: 308 | | CGAGCAAACCCCCCCACCGTCGAGTTTCTCGAC |
| | | GGTCTGCTGCCGGGAGACCGGCAGCGGGGGGG |
| | | TTTGCTCGGTCGCTCGCTCGCTTGCGCGAGAAG |
| | | CCCTCTC |
| SEQ ID | AAV5_Nt.BspQI_BL | GAGAGGGCGAGAAGCGCAAGCGAGCGAGCGA |
| No: 309 | | CCGAGCAAACCCCCCCACCGTCGAGTTTCTCGA |
| | | CGGTCTGCTGCCGGGAGACCGGCAGCGGGGGG |
| | | GTTTGCTCGGTCGCTCGCTCGCTTGCGCTTCTCG |
| | | CCCTCTC |
| SEQ ID | AAV5_Nt.BstNBI_TL | GAGAGGGGGGACAGCGCTGAGCGAGCGAGCG |
| No: 310 | | ACCGAGCAAACCCCCCCACCGTCGAGTTTCTCG |
| | | ACGGTCTGCTGCCGGGAGACCGGCAGCGGGGG |
| | | GGTTTGCTCGGTCGCTCGCTCGCTCAGCGCTGT |
| | | CCCCCCTCTC |
| SEQ ID | AAV5_Nt.BstNBI_BL | GAGAGGGGGCTCAGCGCAAGCGAGCGAGCGAC |
| No: 311 | | CGAGCAAACCCCCCCACCGTCGAGTTTCTCGAC |
| | | GGTCTGCTGCCGGGAGACCGGCAGCGGGGGGG |
| | | TTTGCTCGGTCGCTCGCTCGCTTGCGCTGAGCC |
| | | CCCTCTC |
| SEQ ID | wt_AAV7 | AACCGGTGAGGGAGATACGCGCGAGCGAGCGA |
| No: 312 | | GCCACCCCGGACGCCTGGTTTCCAGGCGTCTGC |
| | | CGTCTCGAGACGAGACGGCCGGGGTGGCTCGC |
| | | TCGCTCGCGCGTATCTCCCTCACCGGTT |
| SEQ ID | AAV7_Nb.BbvCI_BL | AACCGGTGAGGGGGAGTCGCGCGAGCGAGCGA |
| No: 313 | | GCCACCCCGGACGCCTGGTTTCCAGGCGTCTGC |
| | | CGTCTCGAGACGAGACGGCCGGGGTGGCTCGC |
| | | TCGCTCGCGCGACTCCCCCTCACCGGTT |
| SEQ ID | AAV7_Nb.BbvCI_TL | AACCGGTGAGGGCGACTCCCGCGAGCGAGCGA |
| No: 314 | | GCCACCCCGGACGCCTGGTTTCCAGGCGTCTGC |
| | | CGTCTCGAGACGAGACGGCCGGGGTGGCTCGC |
| | | TCGCTCGCGGGAGTCGCCCTCACCGGTT |
| SEQ ID | AAV7_Nb.BsmI_BL | AACCGGTGAGGGACTTACGCGCGAGCGAGCGA |
| No: 315 | | GCCACCCCGGACGCCTGGTTTCCAGGCGTCTGC |
| | | CGTCTCGAGACGAGACGGCCGGGGTGGCTCGC |
| | | TCGCTCGCGCGTAAGTCCCTCACCGGTT |
| SEQ ID | AAV7_Nb.BsmI_TL | AACCGGTGAGGGAGATACGCGTAAGCGAGCGA |
| No: 316 | | GCCACCCCGGACGCCTGGTTTCCAGGCGTCTGC |
| | | CGTCTCGAGACGAGACGGCCGGGGTGGCTCGC |
| | | TCGCTTACGCGTATCTCCCTCACCGGTT |
| SEQ ID | AAV7_Nb.BsrDI_BL | AACCGGTGAGGGCGTTACGCGCGAGCGAGCGA |
| No: 317 | | GCCACCCCGGACGCCTGGTTTCCAGGCGTCTGC |
| | | CGTCTCGAGACGAGACGGCCGGGGTGGCTCGC |
| | | TCGCTCGCGCGTAACGCCCTCACCGGTT |
| SEQ ID | AAV7_Nb.BsrDI_TL | AACCGGTGAGGGAGTAACGCGCGAGCGAGCGA |
| No: 318 | | GCCACCCCGGACGCCTGGTTTCCAGGCGTCTGC |
| | | CGTCTCGAGACGAGACGGCCGGGGTGGCTCGC |
| | | TCGCTCGCGCGTTACTCCCTCACCGGTT |
| SEQ ID | AAV7_Nb.BssSI_BL | AACCGGTGCTCGAGATACGCGCGAGCGAGCGA |
| No: 319 | | GCCACCCCGGACGCCTGGTTTCCAGGCGTCTGC |
| | | CGTCTCGAGACGAGACGGCCGGGGTGGCTCGC |
| | | TCGCTCGCGCGTATCTCGAGCACCGGTT |
| SEQ ID | AAV7_Nb.BssSI_TL | AACCGGTGAGGGAGCACCGCGCGAGCGAGCGA |
| No: 320 | | GCCACCCCGGACGCCTGGTTTCCAGGCGTCTGC |
| | | CGTCTCGAGACGAGACGGCCGGGGTGGCTCGC |
| | | TCGCTCGCGCGGTGCTCCCTCACCGGTT |
| SEQ ID | AAV7_Nb.BtsI_BL | AACCGGTGAGGGCGTCACGCGCGAGCGAGCGA |
| No: 321 | | GCCACCCCGGACGCCTGGTTTCCAGGCGTCTGC |
| | | CGTCTCGAGACGAGACGGCCGGGGTGGCTCGC |
| | | TCGCTCGCGCGTGACGCCCTCACCGGTT |
| SEQ ID | AAV7_Nb.BtsI_TL | AACCGGTGAGGGAGTGACGCGCGAGCGAGCGA |
| No: 322 | | GCCACCCCGGACGCCTGGTTTCCAGGCGTCTGC |
| | | CGTCTCGAGACGAGACGGCCGGGGTGGCTCGC |
| | | TCGCTCGCGCGTCACTCCCTCACCGGTT |
| SEQ ID | AAV7_Nt.AlwI_BL | AACCGGTGAGGGAGCTAGGCGCGAGCGAGCGA |
| No: 323 | | GCCACCCCGGACGCCTGGTTTCCAGGCGTCTGC |
| | | CGTCTCGAGACGAGACGGCCGGGGTGGCTCGC |
| | | TCGCTCGCGCCTAGCTCCCTCACCGGTT |
| SEQ ID | AAV7_Nt.AlwI_BL | AACCGGTGACCTAGATACGCGCGAGCGAGCGA |
| No: 324 | | GCCACCCCGGACGCCTGGTTTCCAGGCGTCTGC |
| | | CGTCTCGAGACGAGACGGCCGGGGTGGCTCGC |
| | | TCGCTCGCGCGTATCTAGGTCACCGGTT |
| SEQ ID | AAV7_Nt.BbvCI_TL | AACCGGTGAGGGCGACTCCCGCGAGCGAGCGA |
| No: 325 | | GCCACCCCGGACGCCTGGTTTCCAGGCGTCTGC |
| | | CGTCTCGAGACGAGACGGCCGGGGGGCTCGC |
| | | TCGCTCGCGGGAGTCGCCCTCACCGGTT |
| SEQ ID | AAV7_Nt.BbvCI_BL | AACCGGTGAGGGGGAGTCGCGCGAGCGAGCGA |
| No: 326 | | GCCACCCCGGACGCCTGGTTTCCAGGCGTCTGC |
| | | CGTCTCGAGACGAGACGGCCGGGGTGGCTCGC |
| | | TCGCTCGCGCGACTCCCCCTCACCGGTT |
| SEQ ID | AAV7_Nt.BsmAI_TL | AACCGGTGACTCTGATACGCGCGAGCGAGCGA |
| No: 327 | | GCCACCCCGGACGCCTGGTTTCCAGGCGTCTGC |
| | | CGTCTCGAGACGAGACGGCCGGGGGGCTCGC |
| | | TCGCTCGCGCGTATCAGAGTCACCGGTT |
| SEQ ID | AAV7_Nt.BsmAI_BL | AACCGGTGAGGCAGAGACGCGCGAGCGAGCGA |
| No: 328 | | GCCACCCCGGACGCCTGGTTTCCAGGCGTCTGC |
| | | CGTCTCGAGACGAGACGGCCGGGGTGGCTCGC |
| | | TCGCTCGCGCGTCTCTGCCTCACCGGTT |
| SEQ ID | AAV7_Nt.BspQI_TL | AACCGGTGAGGGCTTCTCGCGCGAGCGAGCGA |
| No: 329 | | GCCACCCCGGACGCCTGGTTTCCAGGCGTCTGC |
| | | CGTCTCGAGACGAGACGGCCGGGGTGGCTCGC |
| | | TCGCTCGCGCGAGAAGCCCTCACCGGTT |
| SEQ ID | AAV7_Nt.BspQI_BL | AACCGGTGAGGGCGAGAAGCGCGAGCGAGCG |
| No: 330 | | AGCCACCCCGGACGCCTGGTTTCCAGGCGTCTG |
| | | CCGTCTCGAGACGAGACGGCCGGGGTGGCTCG |
| | | CTCGCTCGCGCTTCTCGCCCTCACCGGTT |
| SEQ ID | AAV7_Nt.BstNBI_TL | AACCGGTGAGGGAGATACGCGCTGAGCGAGCG |
| No: 331 | | AGCCACCCCGGACGCCTGGTTTCCAGGCGTCTG |
| | | CCGTCTCGAGACGAGACGGCCGGGGGGCTCG |
| | | CTCGCTCAGCGCGTATCTCCCTCACCGGTT |
| SEQ ID | AAV7_Nt.BstNBI_BL | AACCGCTCAGGGAGATACGCGCGAGCGAGCGA |
| No: 332 | | GCCACCCCGGACGCCTGGTTTCCAGGCGTCTGC |
| | | CGTCTCGAGACGAGACGGCCGGGGTGGCTCGC |
| | | TCGCTCGCGCGTATCTCCCTGAGCGGTT |

The first, second, third, and fourth restriction sites for nicking endonuclease can be arranged in various configurations. In some embodiments, the first and the second restriction sites for nicking endonuclease are at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, or at least 200 nucleotides apart. In other embodiments, the first and the second restriction sites for nicking endonuclease are about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 105, about 110, about 115, about 120, about 125, about 130, about 135, about 140, about 145, about 150, about 155, about 160, about 165, about 170, about 175, about 180, about 185, about 190, about 195, or about 200, or more nucleotides apart. In other embodiments, the nucleotide sequence between the first and the second restriction sites for nicking endonuclease can comprise from about 10 to about 500 nucleotides, such as, for example, from about 10 to about 250, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 105, about 110, about 115, about 120, about 125, about 130, about 135, about 140, about 145, about 150, about 155, about 160, about 165, about 170, about 175, about 180, about 185, about 190, about 195, or about 200 nucleotides apart.

Similarly, in certain embodiments, the third and the fourth restriction sites for nicking endonuclease are at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, or at least 200 nucleotides apart. In further embodiments, the third and the fourth restriction sites for nicking endonuclease are about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 105, about 110, about 115, about 120, about 125, about 130, about 135, about 140, about 145, about 150, about 155, about 160, about 165, about 170, about 175, about 180, about 185, about 190, about 195, or about 200 nucleotides apart.

The disclosure provides that the overhang described in Sections 3, 5.2 (including 5.3.3), and 5.4 (including 5.4.1) can be the result of the nicking at the first and second restriction sites by nicking endonucleases and denaturing as described in Sections 3 and 5.2 (including 5.3.3). Thus, in some embodiments, the overhang resulted from the nicking at the first and second restriction sites can be the same length as the first and second restriction sites are apart (in number of nucleotides) as described in the preceding paragraphs of this Section (Section 5.4.2). As the nicking endonucleases can cut the DNA within or outside the restriction sites for the nicking endonucleases, in certain embodiments, the overhang resulted from the nicking at the first and second restriction sites can be longer or shorter than the first and second restriction sites are apart by at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides. In other embodiments, the overhang resulted from the nicking at the first and second restriction sites can be longer or shorter than the first and second restriction sites are apart by about 10, about 11, about 12, about 13, about 14, about 15, about 16, about 17, about 18, about 19, about 20, about 21, about 22, about 23, about 24, about 25, about 26, about 27, about 28, about 29, or about 30 nucleotides.

Similarly, the disclosure provides that the overhang described in Sections 3, 5.2 (including 5.3.3), and 5.4 (including 5.4.1) can be the result of the nicking at the third and fourth restriction sites by nicking endonucleases and denaturing as described in Sections 3 and 5.2 (including 5.3.3). Thus, in some embodiments, the overhang resulted from the nicking at the third and fourth restriction sites can be the same length as the third and fourth restriction sites are apart (in number of nucleotides) as described in the preceding paragraphs of this Section (Section 5.4.2). As the nicking endonucleases can cut the DNA within or outside the restriction sites for the nicking endonucleases, in certain embodiments, the overhang resulted from the nicking at the third and fourth restriction sites can be longer or shorter than the third and fourth restriction sites are apart by at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides. In other embodiments, the overhang resulted from the nicking at the third and fourth restriction sites can be longer or shorter than the third and fourth restriction sites are apart by about 10, about 11, about 12, about 13, about 14, about 15, about 16, about 17, about 18, about 19, about 20, about 21, about 22, about 23, about 24, about 25, about 26, about 27, about 28, about 29, or about 30 nucleotides.

As is clear from the description in Sections 3 and 5.5 and this Section (Section 5.4), the DNA molecules provided herein comprise an expression cassette. In some embodiments, the expression cassette is located between the first and second restriction sites for nicking endonuclease(s) at one end and the third and fourth restriction sites for nicking endonuclease(s) at the other end. In other embodiments, the expression cassette is located within the dsDNA segment of the DNA molecules produced by performing the method steps a to d as described in Sections 3 and 5.2, including the denaturing step described in Section 5.3.3 to provide two ssDNA overhangs. In certain embodiments, the first, second, third, and fourth restriction sites for the nicking endonucleases are arranged such that the length of the dsDNA segment described in this paragraph is at least 0.2 kb, at least 0.3 kb, at least 0.4 kb, at least 0.5 kb, at least 0.6, at least kb, at least 0.7 kb, at least 0.8 kb, at least 0.9 kb, at least 1 kb, at least 1.5 kb, at least 2 kb, at least 2.5 kb, at least 3 kb, at least 3.5 kb, at least 4 kb, at least 4.5 kb, at least 5 kb, at least 5.5 kb, at least 6 kb, at least 6.5 kb, at least 7 kb, at least 7.5 kb, at least 8 kb, at least 8.5 kb, at least 9 kb, at least 9.5 kb, or at least 10 kb. In other embodiments, the first, second, third, and fourth restriction sites for the nicking endonucleases are arranged such that the length of the dsDNA segment described in this paragraph is about 0.2 kb, about 0.3 kb, about 0.4 kb, about 0.5 kb, about 0.6, about kb, about 0.7 kb, about 0.8 kb, about 0.9 kb, about 1 kb, about 1.5 kb, about 2 kb, about 2.5 kb, about 3 kb, about 3.5 kb, about 4 kb, about 4.5 kb, about 5 kb, about 5.5 kb, about 6 kb, about 6.5 kb, about 7 kb, about 7.5 kb, about 8 kb, about 8.5 kb, about 9 kb, about 9.5 kb, or about 10 kb.

As described in Section 5.3.4, incubation with nicking endonucleases will result in a first nick corresponding to the first restriction site for the nicking endonuclease, a second nick corresponding to the second restriction site for the nicking endonuclease, a third nick corresponding to the third restriction site for the nicking endonuclease, and/or a fourth nick corresponding to the fourth restriction site for the nicking endonuclease. The disclosure provides that the first, second, third, and/or fourth nicks can be at various positions relative to the inverted repeat. In one embodiment, the first nick is within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides from the 5′ nucleotide of the ITR closing base pair of the first inverted repeat. In another embodiment, the first nick is within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides from the 3′ nucleotide of the ITR closing base pair of the first inverted repeat. In yet another embodiment, the second nick is within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides from the 5′ nucleotide of the ITR closing base pair of the first inverted repeat. In a further embodiment, the second nick is within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides from the 3′ nucleotide of the ITR closing base pair of the first inverted repeat. In one embodiment, the third nick is within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides from the 5′ nucleotide of the ITR closing base pair of the second inverted repeat. In another embodiment, the third nick is within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides from the 3′ nucleotide of the ITR closing base pair of the second inverted repeat. In yet another embodiment, the fourth nick is within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides from the 5′ nucleotide of the ITR closing base pair of the second inverted repeat. In a further embodiment, the fourth nick is within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides from the 3′ nucleotide of the ITR closing base pair of the second inverted repeat. In some embodiments, any, or any combinations of the first, second, third, and fourth nicks are inside the inverted repeat. In certain embodiments, any, or any combinations of the first, second, third, and fourth nicks are outside the inverted repeat. In some additional embodiments, the first, second, third, and fourth nicks can have any relative positions amongst themselves, between any of them and the inverted repeat, and/or between any of them and the expression cassette as described in this Section (Section 5.4.2), in any combination or permutation. In some further embodiments, the first, second, third, and fourth restriction sites for nicking endonucleases can have any relative positions amongst themselves, between any of them and the inverted repeat, and/or between any of them and the expression cassette as described in this Section (Section 5.4.2), in any combination or permutation.

### 5.4.3 Expression Cassette Encoding FVIII

The DNA molecules provided herein comprise at least one an expression cassette. An “expression cassette” is a nucleic acid molecule or a part of nucleic acid molecule containing sequences or other information that directs the cellular machinery to make RNA and protein. In some embodiments, an expression cassette comprises a promoter sequence. In certain embodiments, an expression cassette comprises a transcription unit. In yet some other embodiments, an expression cassette comprises a promoter operatively linked to a transcription unit. In one embodiment, the transcription unit comprises an open reading frame (ORF). Embodiments for ORFs for use with the methods and compositions provided herein are further described in the last paragraph of this Section (Section 5.4.3). The expression cassette can further comprise features to direct the cellular machinery to make RNA and protein. In one embodiment, the expression cassette comprises a posttranscriptional regulatory element. In another embodiment, the expression cassette further comprises a polyadenylation and/or termination signal. In yet another embodiment, the expression cassette comprises regulatory elements known and used in the art to regulate (promote, inhibit and/or turn on/off the expression of the ORF). Such regulatory elements include, for example, 5′-untranslated region (UTR), 3′-UTR, or both the 5′UTR and the 3′UTR. In some further embodiments, the expression cassette comprises any one or more features provided in this Section (Section 5.4.3) in any combination or permutation.

The expression cassette can comprise a protein coding sequence in its ORF (sense strand). Alternatively, the expression cassette can comprise the complementary sequence of the protein coding ORF (anti-sense strand) and the regulatory components and/or other signals for the cellular machinery to produce a sense strand DNA/RNA and the corresponding protein. In some embodiments, the expression cassette comprises a protein sequence without intron. In other embodiments, the expression cassette comprises a protein sequence with intron, which is removed upon transcription and splicing. The expression cassette can also comprise various numbers of ORFs or transcription units. In one embodiment, the expression cassette comprises 1, 2, 3, 4, 5, 6, 7, 8, 9, 10, 11, 12, 13, 14, 15, 16, 17, 18, 19, or 20 ORFs. In another embodiment, the expression cassette comprises 1, 2, 3, 4, 5, 6, 7, 8, 9, 10, 11, 12, 13, 14, 15, 16, 17, 18, 19, or 20 transcription units.

The human F8 gene encodes a 2351 amino acid protein (SEQ ID 359); accession number P00451) with a molecular mass of approximately 174.8 kDa. The F8 gene is located on chromosome X. The consensus human F8 coding sequence can be found at NCBI Accession No. NM_000132 and translates into SEQ ID NO: 359.

Once Factor VIII is expressed it is secreted into the blood and circulates in an inactive form. The inactive form is typically bound to von Willebrand factor, which stabilizes it. Following a triggering event such as an injury, Factor VIII is activated. The activated protein then interacts with coagulation Factor IX which proteolytically activates Factor X, triggering the coagulation pathway and leading to clotting.

Factor VIII consists of six domains, namely A1-A2-B-A3-C1-C2 as well as three acidic linker regions a1, a2, and a3 (referred to herein as “linker a1”, “linker a2” and “linker a3). Factor VIII has 19 consensus sites for N-linked glycosylation. FVIII is divided into a heavy chain (A1-a1-A2-a2-B) and a light chain (a3-A3-C1-C₂). Factor VIII is expressed and circulates in an inactive form as a heterodimeric complex consisting of the A1-A2-B domains, and A3-C1-C2 domains. Factor VIII is activated by proteolytic cleavage by thrombin. Following thrombin cleavage, Factor VIII forms a heterotrimeric complex consisting of the A1 domain, A2 domain, and A3-C1-C2 domains and undergoes a conformational change, which allows binding to Factor IXa and activation of Factor X. Following activation, Factor VIIIa may undergo further proteolysis, and/or the respective components of the heterotrimeric complex may dissociate from one-another, thereby inactivating Factor VIIIa. It has been demonstrated that the B-domain is not necessary for Factor VIII cofactor activity.

One of skill in the art will understand that the FVIII therapeutic protein (also referred to herein as a therapeutic FVIII protein) includes all splice variants and orthologs of the FVIII protein. Essentially any version of the FVIII therapeutic protein or fragment thereof (e.g., functional fragment) can be encoded by and expressed in and from a hairpin ended DNA vector as described herein. FVIII therapeutic protein includes intact molecules as well as fragments (e.g., functional) thereof. In some embodiments, the FVIII therapeutic protein or fragment thereof is modified compared to wild-type FVIII. Examples of modified FVIII therapeutic protein include, but are not limited to, those expressly described herein.

The term “variant Factor VIII (FVIII)” refers to a modified FVIII which has been genetically altered as compared to unmodified wild-type FVIII (e.g., SEQ ID NO: 359) or a truncated FVIII. Such a variant can be referred to as a “nucleic acid variant encoding Factor VIII (FVIII).” A particular example of a variant is a CpG reduced nucleic acid encoding FVIII or a functional fragment thereof. The term “variant” need not appear in each instance of a reference made to a CpG reduced nucleic acid encoding FVIII. Likewise, the term “CpG reduced nucleic acid” or the like may omit the term “variant” but it is intended that reference to a “CpG reduced nucleic acid” includes variants at the genetic level.

FVIII constructs having reduced CpG content can exhibit improvements compared to wild-type FVIII or functional fragments thereof in which CpG content has not been reduced. These improvements may be observed even without modifications to the nucleic acid which would result in change in the primary amino acid sequence of the encoded FVIII protein.

The “functional fragment(s)” of FVIII provided herein include modified FVIII fragments such that the modified protein has an amino acid alteration compared to wild-type FVIII but retains some degree of the functionality of the native full-length protein, has increased protein activity as compared to the native full-length protein, and/or has improved protein functionality as compared to the native full-length protein. For example, in certain embodiments, a CpG reduced nucleic acid encoding FVIII or truncated FVIII protein comprises a B-domain deletion as set forth herein, and the expressed protein retains clotting function. In another embodiment, a CpG reduced nucleic acid encoding FVIII or truncated FVIII protein comprises a B domain and/or linker a3deletion as set forth herein, and the expressed protein retains clotting function. In certain embodiments, a variant truncated FVIII may retain a portion of the B-domain. Thus, in certain embodiments, the truncated FVIII comprises a portion of the B-domain.

In some embodiments, the hairpinned DNA molecule for the expression of the FVIII protein provide an advantage over traditional AAV vectors, as there is no size constraint for the heterologous nucleic acid sequences encoding a desired protein. Thus, even a full length FVIII protein can be expressed from a single DNA molecule. Thus, the DNA molecules described herein can be used to express a therapeutic FVIII protein in a subject in need thereof, e.g., a subject with Hemophilia A.

TABLE 18
Exemplary Transgenes

| Name | Sequence |
|---|---|
| FVIII | MQIELSTCFFLCLLRFCFSATRRYYLGAVELSWDYMQSDLGELPVDA |
| (accession | RFPPRVPKSFPFNTSVVYKKTLFVEFTDHLFNIAKPRPPWMGLLGPTIQ |
| number | AEVYDTVVITLKNMASHPVSLHAVGVSYWKASEGAEYDDQTSQREK |
| P00451) | EDDKVFPGGSHTYVWQVLKENGPMASDPLCLTYSYLSHVDLVKDLN |
| (SEQ ID | SGLIGALLVCREGSLAKEKTQTLHKFILLFAVFDEGKSWHSETKNSLM |
| NO: 359) | QDRDAASARAWPKMHTVNGYVNRSLPGLIGCHRKSVYWHVIGMGT |
| | TPEVHSIFLEGHTFLVRNHRQASLEISPITFLTAQTLLMDLGQFLLFCHI |
| | SSHQHDGMEAYVKVDSCPEEPQLRMKNNEEAEDYDDDLTDSEMDV |
| | VRFDDDNSPSFIQIRSVAKKHPKTWVHYIAAEEEDWDYAPLVLAPDD |
| | RSYKSQYLNNGPQRIGRKYKKVRFMAYTDETFKTREAIQHESGILGPL |
| | LYGEVGDTLLIIFKNQASRPYNIYPHGITDVRPLYSRRLPKGVKHLKDF |
| | PILPGEIFKYKWTVTVEDGPTKSDPRCLTRYYSSFVNMERDLASGLIGP |
| | LLICYKESVDQRGNQIMSDKRNVILFSVFDENRSWYLTENIQRFLPNP |
| | AGVQLEDPEFQASNIMHSINGYVFDSLQLSVCLHEVAYWYILSIGAQT |
| | DFLSVFFSGYTFKHKMVYEDTLTLFPFSGETVFMSMENPGLWILGCH |
| | NSDFRNRGMTALLKVSSCDKNTGDYYEDSYEDISAYLLSKNNAIEPRS |
| | FSQNSRHPSTRQKQFNATTIPENDIEKTDPWFAHRTPMPKIQNVSSSDL |
| | LMLLRQSPTPHGLSLSDLQEAKYETFSDDPSPGAIDSNNSLSEMTHFRP |
| | QLHHSGDMVFTPESGLQLRLNEKLGTTAATELKKLDFKVSSTSNNLIS |
| | TIPSDNLAAGTDNTSSLGPPSMPVHYDSQLDTTLFGKKSSPLTESGGPL |
| | SLSEENNDSKLLESGLMNSQESSWGKNVSSTESGRLFKGKRAHGPAL |
| | LTKDNALFKVSISLLKTNKTSNNSATNRKTHIDGPSLLIENSPSVWQNI |
| | LESDTEFKKVTPLIHDRMLMDKNATALRLNHMSNKTTSSKNMEMVQ |
| | QKKEGPIPPDAQNPDMSFFKMLFLPESARWIQRTHGKNSLNSGQGPSP |
| | KQLVSLGPEKSVEGQNFLSEKNKVVVGKGEFTKDVGLKEMVFPSSRN |
| | LFLTNLDNLHENNTHNQEKKIQEEIEKKETLIQENVVLPQIHTVTGTK |
| | NFMKNLFLLSTRQNVEGSYDGAYAPVLQDFRSLNDSTNRTKKHTAHF |
| | SKKGEEENLEGLGNQTKQIVEKYACTTRISPNTSQQNFVTQRSKRALK |
| | QFRLPLEETELEKRIIVDDTSTQWSKNMKHLTPSTLTQIDYNEKEKGAI |
| | TQSPLSDCLTRSHSIPQANRSPLPIAKVSSFPSIRPIYLTRVLFQDNSSHL |
| | PAASYRKKDSGVQESSHFLQGAKKNNLSLAILTLEMTGDQREVGSLG |
| | TSATNSVTYKKVENTVLPKPDLPKTSGKVELLPKVHIYQKDLFPTETS |
| | NGSPGHLDLVEGSLLQGTEGAIKWNEANRPGKVPFLRVATESSAKTP |
| | SKLLDPLAWDNHYGTQIPKEEWKSQEKSPEKTAFKKKDTILSLNACES |
| | NHAIAAINEGQNKPEIEVTWAKQGRTERLCSQNPPVLKRHQREITRTT |
| | LQSDQEEIDYDDTISVEMKKEDFDIYDEDENQSPRSFQKKTRHYFIAA |
| | VERLWDYGMSSSPHVLRNRAQSGSVPQFKKVVFQEFTDGSFTQPLYR |
| | GELNEHLGLLGPYIRAEVEDNIMVTFRNQASRPYSFYSSLISYEEDQRQ |
| | GAEPRKNFVKPNETKTYFWKVQHHMAPTKDEFDCKAWAYFSDVDL |
| | EKDVHSGLIGPLLVCHTNTLNPAHGRQVTVQEFALFFTIFDETKSWYF |
| | TENMERNCRAPCNIQMEDPTFKENYRFHAINGYIMDTLPGLVMAQDQ |
| | RIRWYLLSMGSNENIHSIHFSGHVFTVRKKEEYKMALYNLYPGVFET |
| | VEMLPSKAGIWRVECLIGEHLHAGMSTLFLVYSNKCQTPLGMASGHI |
| | RDFQITASGQYGQWAPKLARLHYSGSINAWSTKEPFSWIKVDLLAPM |
| | IIHGIKTQGARQKFSSLYISQFIIMYSLDGKKWQTYRGNSTGTLMVFFG |
| | NVDSSGIKHNIFNPPIIARYIRLHPTHYSIRSTLRMELMGCDLNSCSMPL |
| | GMESKAISDAQITASSYFTNMFATWSPSKARLHLQGRSNAWRPQVNN |
| | PKEWLQVDFQKTMKVTGVTTQGVKSLLTSMYVKEFLISSSQDGHQW |
| | TLFFQNGKVKVFQGNQDSFTPVVNSLDPPLLTRYLRIHPQSWVHQIAL |
| | RMEVLGCEAQDLY |
| Truncated | MQIELSTCFFLCLLRFCFSATRRYYLGAVELSWDYMQSDLGELPVDA |
| F8 v1 | RFPPRVPKSFPFNTSVVYKKTLFVEFTDHLFNIAKPRPPWMGLLGPTIQ |
| (SEQ ID | AEVYDTVVITLKNMASHPVSLHAVGVSYWKASEGAEYDDQTSQREK |
| NO: 354) | EDDKVFPGGSHTYVWQVLKENGPMASDPLCLTYSYLSHVDLVKDLN |
| | SGLIGALLVCREGSLAKEKTQTLHKFILLFAVFDEGKSWHSETKNSLM |
| | QDRDAASARAWPKMHTVNGYVNRSLPGLIGCHRKSVYWHVIGMGT |
| | TPEVHSIFLEGHTFLVRNHRQASLEISPITFLTAQTLLMDLGQFLLSCHI |
| | SSHQHDGMEAYVKVDSCPEEPQLRMKNNEEAEDYDDDLTDSEMDV |
| | VRFDDDNSPSFIQIRSVAKKHPKTWVHYIAAEEEDWDYAPLVLAPDD |
| | RSYKSQYLNNGPQRIGRKYKKVRFMAYTDETFKTREAIQHESGILGPL |
| | LYGEVGDTLLIIFKNQASRPYNIYPHGITDVRPLYSRRLPKGVKHLKDF |
| | PILPGEIFKYKWTVTVEDGPTKSDPRCLTRYYSSFVNMERDLASGLIGP |
| | LLICYKESVDQRGNQIMSDKRNVILFSVFDENRSWYLTENIQRFLPNP |
| | AGVQLEDPEFQASNIMHSINGYVFDSLQLSVCLHEVAYWYILSIGAQT |
| | DFLSVFFSGYTFKHKMVYEDTLTLFPFSGETVFMSMENPGLWILGCH |
| | NSDFRNRGMTALLKVSSCDKNTGDYYEDSYEDISAYLLSKNNAIEPRS |
| | FSQNSRHPSTRQKQFNATTIPENDIEKTDPWFAHRTPMPKIQNVSSSDL |
| | LMLLRQSPTPHGLSLSDLQEAKYETFSDDPSPGAIDSNNSLSEMTHFRP |
| | QLHHSGDMVFTPESGLQLRLNEKLGTTAATELKKLDFKVSSTSNNLIS |
| | TIPSDNLAAGTDNTSSLGPPSMPVHYDSQLDTTLFGKKSSPLTESGGPL |
| | SLSEENNDSKLLESGLMNSQESSWGKNVSQREITRTTLQSDQEEIDYD |
| | DTISVEMKKEDFDIYDEDENQSPRSFQKKTRHYFIAAVERLWDYGMS |
| | SSPHVLRNRAQSGSVPQFKKVVFQEFTDGSFTQPLYRGELNEHLGLLG |
| | PYIRAEVEDNIMVTFRNQASRPYSFYSSLISYEEDQRQGAEPRKNFVKP |
| | NETKTYFWKVQHHMAPTKDEFDCKAWAYFSDVDLEKDVHSGLIGPL |
| | LVCHTNTLNPAHGRQVTVQEFALFFTIFDETKSWYFTENMERNCRAP |
| | CNIQMEDPTFKENYRFHAINGYIMDTLPGLVMAQDQRIRWYLLSMGS |
| | NENIHSIHFSGHVFTVRKKEEYKMALYNLYPGVFETVEMLPSKAGIW |
| | RVECLIGEHLHAGMSTLFLVYSNKCQTPLGMASGHIRDFQITASGQYG |
| | QWAPKLARLHYSGSINAWSTKEPFSWIKVDLLAPMIIHGIKTQGARQK |
| | FSSLYISQFIIMYSLDGKKWQTYRGNSTGTLMVFFGNVDSSGIKHNIFN |
| | PPIIARYIRLHPTHYSIRSTLRMELMGCDLNSCSMPLGMESKAISDAQIT |
| | ASSYFTNMFATWSPSKARLHLQGRSNAWRPQVNNPKEWLQVDFQKT |
| | MKVTGVTTQGVKSLLTSMYVKEFLISSSQDGHQWTLFFQNGKVKVF |
| | QGNQDSFTPVVNSLDPPLLTRYLRIHPQSWVHQIALRMEVLGCEAQD |
| | LY |
| Truncated | MQIELSTCFFLCLLRFCFSATRRYYLGAVELSWDYMQSDLGELPVDA |
| F8 v2 | RFPPRVPKSFPFNTSVVYKKTLFVEFTDHLFNIAKPRPPWMGLLGPTIQ |
| (SEQ ID | AEVYDTVVITLKNMASHPVSLHAVGVSYWKASEGAEYDDQTSQREK |
| NO: 355) | EDDKVFPGGSHTYVWQVLKENGPMASDPLCLTYSYLSHVDLVKDLN |
| | SGLIGALLVCREGSLAKEKTQTLHKFILLFAVFDEGKSWHSETKNSLM |
| | QDRDAASARAWPKMHTVNGYVNRSLPGLIGCHRKSVYWHVIGMGT |
| | TPEVHSIFLEGHTFLVRNHRQASLEISPITFLTAQTLLMDLGQFLLSCHI |
| | SSHQHDGMEAYVKVDSCPEEPQLRMKNNEEAEDYDDDLTDSEMDV |
| | VRFDDDNSPSFIQIRSVAKKHPKTWVHYIAAEEEDWDYAPLVLAPDD |
| | RSYKSQYLNNGPQRIGRKYKKVRFMAYTDETFKTREAIQHESGILGPL |
| | LYGEVGDTLLIIFKNQASRPYNIYPHGITDVRPLYSRRLPKGVKHLKDF |
| | PILPGEIFKYKWTVTVEDGPTKSDPRCLTRYYSSFVNMERDLASGLIGP |
| | LLICYKESVDQRGNQIMSDKRNVILFSVFDENRSWYLTENIQRFLPNP |
| | AGVQLEDPEFQASNIMHSINGYVFDSLQLSVCLHEVAYWYILSIGAQT |
| | DFLSVFFSGYTFKHKMVYEDTLTLFPFSGETVFMSMENPGLWILGCH |
| | NSDFRNRGMTALLKVSSCDKNTGDYYEDSYEDISAYLLSKNNAIEPRS |
| | FSQNSRHPSTRQKQFNATTIPENDIEKTDPWFAHRTPMPKIQNVSSSDL |
| | LMLLRQSPTPHGLSLSDLQEAKYETFSDDPSPGAIDSNNSLSEMTHFRP |
| | QLHHSGDMVFTPESGLQLRLNEKLGTTAATELKKLDFKVSSTSNNLIS |
| | TIPSDNLAAGTDNTSSLGPPSMPVHYDSQLDTTLFGKKSSPLTESGGPL |
| | SLSEENNDSKLLESGLMNSQESSWGKNVSSTESGRLFKGKRAHGPAL |
| | LTKDNALFKVSISLLKTNKTSNNSATNRKTHIDGPSLLIENSPSVWQNI |
| | LESDTEFKKVTPLIHDRMLMDKNATALRLNHMSNKTTSSKNMEMVQ |
| | QKKEGPIPPDAQNPDMSFFKMLFLPESARWIQRTHGKNSLNSGQGPSP |
| | KQLVSLGPEKSVEGQNFLSEKNKVVVGKGEFTKDVGLKEMVFPSSRN |
| | LFLTNLDNLHENNTHNQEKKIQEEIEKKETLIQENVVLPQIHTVTGTK |
| | NFMKNLFLLSTRQNVEGSYDGAYAPVLQDFRSLNDSTNRTKKHTAHF |
| | SKKGEEENLEGLGNQTKQIVEKYACTTRISPNTSQQNFVTQRSKRALK |
| | QFRLPLEETELEKRIIVDDTSTQWSKNMKHLTPSTLTQIDYNEKEKGAI |
| | TQSPLSDCLTRSHSIPQANRSPLPIAKVSSFPSIRPIYLTRVLFQDNSSHL |
| | PAASYRKKDSGVQESSHFLQGAKKNNLSLAILTLEMTGDQREVGSLG |
| | TSATNSVTYKKVENTVLPKPDLPKTSGKVELLPKVHIYQKDLFPTETS |
| | NGSPGHLDLVEGSLLQGTEGAIKWNEANRPGKVPFLRVATESSAKTP |
| | SKLLDPLAWDNHYGTQIPKEEWKSQEKSPEKTAFKKKDTILSLNACES |
| | NHAIAAINEGQNKPEIEVTWAKQGRTERLCSQNPPVLKRHQREITRTT |
| | LQSDQEEIDYDDTISVEMKKEDFDIYDEDENQSPRSFQKKTRHYFIAA |
| | VERLWDYGMSSSPHVLRNRAQSGSVPQFKKVVFQEFTDGSFTQPLYR |
| | GELNEHLGLLGPYIRAEVEDNIMVTFRNQASRPYSFYSSLISYEEDQRQ |
| | GAEPRKNFVKPNETKTYFWKVQHHMAPTKDEFDCKAWAYFSDVDL |
| | EKDVHSGLIGPLLVCHTNTLNPAHGRQVTVQEFALFFTIFDETKSWYF |
| | TENMERNCRAPCNIQMEDPTFKENYRFHAINGYIMDTLPGLVMAQDQ |
| | RIRWYLLSMGSNENIHSIHFSGHVFTVRKKEEYKMALYNLYPGVFET |
| | VEMLPSKAGIWRVECLIGEHLHAGMSTLFLVYSNKCQTPLGMASGHI |
| | RDFQITASGQYGQWAPKLARLHYSGSINAWSTKEPFSWIKVDLLAPM |
| | IIHGIKTQGARQKFSSLYISQFIIMYSLDGKKWQTYRGNSTGTLMVFFG |
| | NVDSSGIKHNIFNPPIIARYIRLHPTHYSIRSTLRMELMGCDLNSCSMPL |
| | GMESKAISDAQITASSYFTNMFATWSPSKARLHLQGRSNAWRPQVNN |
| | PKEWLQVDFQKTMKVTGVTTQGVKSLLTSMYVKEFLISSSQDGHQW |
| | TLFFQNGKVKVFQGNQDSFTPVVNSLDPPLLTRYLRIHPQSWVHQIAL |
| | RMEVLGCEAQDLY |
| Truncated | MQIELSTCFFLCLLRFCFSATRRYYLGAVELSWDYMQSDLGELPVDA |
| F8 v3 | RFPPRVPKSFPFNTSVVYKKTLFVEFTDHLFNIAKPRPPWMGLLGPTIQ |
| (SEQ ID | AEVYDTVVITLKNMASHPVSLHAVGVSYWKASEGAEYDDQTSQREK |
| NO: 356) | EDDKVFPGGSHTYVWQVLKENGPMASDPLCLTYSYLSHVDLVKDLN |
| | SGLIGALLVCREGSLAKEKTQTLHKFILLFAVFDEGKSWHSETKNSLM |
| | QDRDAASARAWPKMHTVNGYVNRSLPGLIGCHRKSVYWHVIGMGT |
| | TPEVHSIFLEGHTFLVRNHRQASLEISPITFLTAQTLLMDLGQFLLSCHI |
| | SSHQHDGMEAYVKVDSCPEEPQLRMKNNEEAEDYDDDLTDSEMDV |
| | VRFDDDNSPSFIQIRSVAKKHPKTWVHYIAAEEEDWDYAPLVLAPDD |
| | RSYKSQYLNNGPQRIGRKYKKVRFMAYTDETFKTREAIQHESGILGPL |
| | LYGEVGDTLLIIFKNQASRPYNIYPHGITDVRPLYSRRLPKGVKHLKDF |
| | PILPGEIFKYKWTVTVEDGPTKSDPRCLTRYYSSFVNMERDLASGLIGP |
| | LLICYKESVDQRGNQIMSDKRNVILFSVFDENRSWYLTENIQRFLPNP |
| | AGVQLEDPEFQASNIMHSINGYVFDSLQLSVCLHEVAYWYILSIGAQT |
| | DFLSVFFSGYTFKHKMVYEDTLTLFPFSGETVFMSMENPGLWILGCH |
| | NSDFRNRGMTALLKVSSCDKNTGDYYEDSYEDISAYLLSKNNAIEPRS |
| | FSQNPPVLKHHQREITRTTLQSDQEEIDYDDTISVEMKKEDFDIYDEDE |
| | NQSPRSFQKKTRHYFIAAVERLWDYGMSSSPHVLRNRAQSGSVPQFK |
| | KVVFQEFTDGSFTQPLYRGELNEHLGLLGPYIRAEVEDNIMVTFRNQA |
| | SRPYSFYSSLISYEEDQRQGAEPRKNFVKPNETKTYFWKVQHHMAPT |
| | KDEFDCKAWAYFSDVDLEKDVHSGLIGPLLVCHTNTLNPAHGRQVT |
| | VQEFALFFTIFDETKSWYFTENMERNCRAPCNIQMEDPTFKENYRFHA |
| | INGYIMDTLPGLVMAQDQRIRWYLLSMGSNENIHSIHFSGHVFTVRKK |
| | EEYKMALYNLYPGVFETVEMLPSKAGIWRVECLIGEHLHAGMSTLFL |
| | VYSNKCQTPLGMASGHIRDFQITASGQYGQWAPKLARLHYSGSINAW |
| | STKEPFSWIKVDLLAPMIIHGIKTQGARQKFSSLYISQFIIMYSLDGKK |
| | WQTYRGNSTGTLMVFFGNVDSSGIKHNIFNPPIIARYIRLHPTHYSIRST |
| | LRMELMGCDLNSCSMPLGMESKAISDAQITASSYFTNMFATWSPSKA |
| | RLHLQGRSNAWRPQVNNPKEWLQVDFQKTMKVTGVTTQGVKSLLT |
| | SMYVKEFLISSSQDGHQWTLFFQNGKVKVFQGNQDSFTPVVNSLDPP |
| | LLTRYLRIHPQSWVHQIALRMEVLGCEAQDLY |
| Truncated | MQIELSTCFFLCLLRFCFSATRRYYLGAVELSWDYMQSDLGELPVDA |
| F8 v4 | RFPPRVPKSFPFNTSVVYKKTLFVEFTDHLFNIAKPRPPWMGLLGPTIQ |
| (SEQ ID | AEVYDTVVITLKNMASHPVSLHAVGVSYWKASEGAEYDDQTSQREK |
| NO: 357) | EDDKVFPGGSHTYVWQVLKENGPMASDPLCLTYSYLSHVDLVKDLN |
| | SGLIGALLVCREGSLAKEKTQTLHKFILLFAVFDEGKSWHSETKNSLM |
| | QDRDAASARAWPKMHTVNGYVNRSLPGLIGCHRKSVYWHVIGMGT |
| | TPEVHSIFLEGHTFLVRNHRQASLEISPITFLTAQTLLMDLGQFLLSCHI |
| | SSHQHDGMEAYVKVDSCPEEPQLRMKNNEEAEDYDDDLTDSEMDV |
| | VRFDDDNSPSFIQIRSVAKKHPKTWVHYIAAEEEDWDYAPLVLAPDD |
| | RSYKSQYLNNGPQRIGRKYKKVRFMAYTDETFKTREAIQHESGILGPL |
| | LYGEVGDTLLIIFKNQASRPYNIYPHGITDVRPLYSRRLPKGVKHLKDF |
| | PILPGEIFKYKWTVTVEDGPTKSDPRCLTRYYSSFVNMERDLASGLIGP |
| | LLICYKESVDQRGNQIMSDKRNVILFSVFDENRSWYLTENIQRFLPNP |
| | AGVQLEDPEFQASNIMHSINGYVFDSLQLSVCLHEVAYWYILSIGAQT |
| | DFLSVFFSGYTFKHKMVYEDTLTLFPFSGETVFMSMENPGLWILGCH |
| | NSDFRNRGMTALLKVSSCDKNTGDYYEDSYEDISAYLLSKNNAIEPRS |
| | FSQNSRHPSTRQKQFNATTIPENDIEKTDPWFAHRTPMPKIQNVSSSDL |
| | LMLLRQSPTPHGLSLSDLQEAKYETFSDDPSPGAIDSNNSLSEMTHFRP |
| | QLHHSGDMVFTPESGLQLRLNEKLGTTAATELKKLDFKVSSTSNNLIS |
| | TIPSDNLAAGTDNTSSLGPPSMPVHYDSQLDTTLFGKKSSPLTESGGPL |
| | SLSEENNDSKLLESGLMNSQESSWGKNVSRSFQKKTRHYFIAAVERL |
| | WDYGMSSSPHVLRNRAQSGSVPQFKKVVFQEFTDGSFTQPLYRGELN |
| | EHLGLLGPYIRAEVEDNIMVTFRNQASRPYSFYSSLISYEEDQRQGAEP |
| | RKNFVKPNETKTYFWKVQHHMAPTKDEFDCKAWAYFSDVDLEKDV |
| | HSGLIGPLLVCHTNTLNPAHGRQVTVQEFALFFTIFDETKSWYFTENM |
| | ERNCRAPCNIQMEDPTFKENYRFHAINGYIMDTLPGLVMAQDQRIRW |
| | YLLSMGSNENIHSIHFSGHVFTVRKKEEYKMALYNLYPGVFETVEML |
| | PSKAGIWRVECLIGEHLHAGMSTLFLVYSNKCQTPLGMASGHIRDFQI |
| | TASGQYGQWAPKLARLHYSGSINAWSTKEPFSWIKVDLLAPMIIHGIK |
| | TQGARQKFSSLYISQFIIMYSLDGKKWQTYRGNSTGTLMVFFGNVDSS |
| | GIKHNIFNPPIIARYIRLHPTHYSIRSTLRMELMGCDLNSCSMPLGMES |
| | KAISDAQITASSYFTNMFATWSPSKARLHLQGRSNAWRPQVNNPKEW |
| | LQVDFQKTMKVTGVTTQGVKSLLTSMYVKEFLISSSQDGHQWTLFFQ |
| | NGKVKVFQGNQDSFTPVVNSLDPPLLTRYLRIHPQSWVHQIALRMEV |
| | LGCEAQDLY |
| Truncated | MQIELSTCFFLCLLRFCFSATRRYYLGAVELSWDYMQSDLGELPVDA |
| F8 (partial | RFPPRVPKSFPFNTSVVYKKTLFVEFTDHLFNIAKPRPPWMGLLGPTIQ |
| deletion of | AEVYDTVVITLKNMASHPVSLHAVGVSYWKASEGAEYDDQTSQREK |
| B domain | EDDKVFPGGSHTYVWQVLKENGPMASDPLCLTYSYLSHVDLVKDLN |
| and linker | SGLIGALLVCREGSLAKEKTQTLHKFILLFAVFDEGKSWHSETKNSLM |
| a3) (SEQ | QDRDAASARAWPKMHTVNGYVNRSLPGLIGCHRKSVYWHVIGMGT |
| ID NO: | TPEVHSIFLEGHTFLVRNHRQASLEISPITFLTAQTLLMDLGQFLLSCHI |
| 358) | SSHQHDGMEAYVKVDSCPEEPQLRMKNNEEAEDYDDDLTDSEMDV |
| | VRFDDDNSPSFIQIRSVAKKHPKTWVHYIAAEEEDWDYAPLVLAPDD |
| | RSYKSQYLNNGPQRIGRKYKKVRFMAYTDETFKTREAIQHESGILGPL |
| | LYGEVGDTLLIIFKNQASRPYNIYPHGITDVRPLYSRRLPKGVKHLKDF |
| | PILPGEIFKYKWTVTVEDGPTKSDPRCLTRYYSSFVNMERDLASGLIGP |
| | LLICYKESVDQRGNQIMSDKRNVILFSVFDENRSWYLTENIQRFLPNP |
| | AGVQLEDPEFQASNIMHSINGYVFDSLQLSVCLHEVAYWYILSIGAQT |
| | DFLSVFFSGYTFKHKMVYEDTLTLFPFSGETVFMSMENPGLWILGCH |
| | NSDFRNRGMTALLKVSSCDKNTGDYYEDSYEDISAYLLSKNNAIEPRS |
| | FSQNSRHPSTRQKQFNATTIPENDIEKTDPWFAHRTPMPKIQNVSSSDL |
| | LMLLRQSPTPHGLSLSDLQEAKYETFSDDPSPGAIDSNNSLSEMTHFRP |
| | QLHHSGDMVFTPESGLQLRLNEKLGTTAATELKKLDFKVSSTSNNLIS |
| | TIPSDNLAAGTDNTSSLGPPSMPVHYDSQLDTTLFGKKSSPLTESGGPL |
| | SLSEENNDSKLLESGLMNSQESSWGKNVSQEEIDYDDTISVEMKKEDF |
| | DIYDEDENQSPRSFQKKTRHYFIAAVERLWDYGMSSSPHVLRNRAQS |
| | GSVPQFKKVVFQEFTDGSFTQPLYRGELNEHLGLLGPYIRAEVEDNIM |
| | VTFRNQASRPYSFYSSLISYEEDQRQGAEPRKNFVKPNETKTYFWKV |
| | QHHMAPTKDEFDCKAWAYFSDVDLEKDVHSGLIGPLLVCHTNTLNP |
| | AHGRQVTVQEFALFFTIFDETKSWYFTENMERNCRAPCNIQMEDPTF |
| | KENYRFHAINGYIMDTLPGLVMAQDQRIRWYLLSMGSNENIHSIHFSG |
| | HVFTVRKKEEYKMALYNLYPGVFETVEMLPSKAGIWRVECLIGEHLH |
| | AGMSTLFLVYSNKCQTPLGMASGHIRDFQITASGQYGQWAPKLARLH |
| | YSGSINAWSTKEPFSWIKVDLLAPMIIHGIKTQGARQKFSSLYISQFIIM |
| | YSLDGKKWQTYRGNSTGTLMVFFGNVDSSGIKHNIFNPPIIARYIRLHP |
| | THYSIRSTLRMELMGCDLNSCSMPLGMESKAISDAQITASSYFTNMFA |
| | TWSPSKARLHLQGRSNAWRPQVNNPKEWLQVDFQKTMKVTGVTTQ |
| | GVKSLLTSMYVKEFLISSSQDGHQWTLFFQNGKVKVFQGNQDSFTPV |
| | VNSLDPPLLTRYLRIHPQSWVHQIALRMEVLGCEAQDLY |
| Native | atgcaaatagagctctccacctgcttctttctgtgccttttgcgattctgctttagtgccaccagaagatactacctg |
| FVIII | ggtgcagtggaactgtcatgggactatatgcaaagtgatctcggtgagctgcctgtggacgcaagatttcctcct |
| isoform a | agagtgccaaaatcttttccattcaacacctcagtcgtgtacaaaaagactctgtttgtagaattcacggatcacct |
| (NM_ | tttcaacatcgctaagccaaggccaccctggatgggtctgctaggtcctaccatccaggctgaggtttatgatac |
| 000132) | agtggtcattacacttaagaacatggcttcccatcctgtcagtcttcatgctgttggtgtatcctactggaaagcttc |
| | tgagggagctgaatatgatgatcagaccagtcaaagggagaaagaagatgataaagtcttccctggtggaag |
| | ccatacatatgtctggcaggtcctgaaagagaatggtccaatggcctctgacccactgtgccttacctactcatat |
| | ctttctcatgtggacctggtaaaagacttgaattcaggcctcattggagccctactagtatgtagagaagggagt |
| | ctggccaaggaaaagacacagaccttgcacaaatttatactactttttgctgtatttgatgaagggaaaagttggc |
| | actcagaaacaaagaactccttgatgcaggatagggatgctgcatctgctcgggcctggcctaaaatgcacac |
| | agtcaatggttatgtaaacaggtctctgccaggtctgattggatgccacaggaaatcagtctattggcatgtgatt |
| | ggaatgggcaccactcctgaagtgcactcaatattcctcgaaggtcacacatttcttgtgaggaaccatcgcca |
| | ggcgtccttggaaatctcgccaataactttccttactgctcaaacactcttgatggaccttggacagtttctactgtt |
| | ttgtcatatctcttcccaccaacatgatggcatggaagcttatgtcaaagtagacagctgtccagaggaacccca |
| | actacgaatgaaaaataatgaagaagcggaagactatgatgatgatcttactgattctgaaatggatgtggtcag |
| | gtttgatgatgacaactctccttcctttatccaaattcgctcagttgccaagaagcatcctaaaacttgggtacatta |
| | cattgctgctgaagaggaggactgggactatgctcccttagtcctcgcccccgatgacagaagttataaaagtc |
| | aatatttgaacaatggccctcagcggattggtaggaagtacaaaaaagtccgatttatggcatacacagatgaa |
| | acctttaagactcgtgaagctattcagcatgaatcaggaatcttgggacctttactttatggggaagttggagaca |
| | cactgttgattatatttaagaatcaagcaagcagaccatataacatctaccctcacggaatcactgatgtccgtcc |
| | tttgtattcaaggagattaccaaaaggtgtaaaacatttgaaggattttccaattctgccaggagaaatattcaaat |
| | ataaatggacagtgactgtagaagatgggccaactaaatcagatcctcggtgcctgacccgctattactctagtt |
| | tcgttaatatggagagagatctagcttcaggactcattggccctctcctcatctgctacaaagaatctgtagatca |
| | aagaggaaaccagataatgtcagacaagaggaatgtcatcctgttttctgtatttgatgagaaccgaagctggta |
| | cctcacagagaatatacaacgctttctccccaatccagctggagtgcagcttgaggatccagagttccaagcct |
| | ccaacatcatgcacagcatcaatggctatgtttttgatagtttgcagttgtcagtttgtttgcatgaggtggcatact |
| | ggtacattctaagcattggagcacagactgacttcctttctgtcttcttctctggatataccttcaaacacaaaatgg |
| | tctatgaagacacactcaccctattcccattctcaggagaaactgtcttcatgtcgatggaaaacccaggtctatg |
| | gattctggggtgccacaactcagactttcggaacagaggcatgaccgccttactgaaggtttctagttgtgacaa |
| | gaacactggtgattattacgaggacagttatgaagatatttcagcatacttgctgagtaaaaacaatgccattgaa |
| | ccaagaagcttctcccagaattcaagacaccctagcactaggcaaaagcaatttaatgccaccacaattccaga |
| | aaatgacatagagaagactgacccttggtttgcacacagaacacctatgcctaaaatacaaaatgtctcctctag |
| | tgatttgttgatgctcttgcgacagagtcctactccacatgggctatccttatctgatctccaagaagccaaatatg |
| | agactttttctgatgatccatcacctggagcaatagacagtaataacagcctgtctgaaatgacacacttcaggc |
| | cacagctccatcacagtggggacatggtatttacccctgagtcaggcctccaattaagattaaatgagaaactg |
| | gggacaactgcagcaacagagttgaagaaacttgatttcaaagtttctagtacatcaaataatctgatttcaacaa |
| | ttccatcagacaatttggcagcaggtactgataatacaagttccttaggacccccaagtatgccagttcattatga |
| | tagtcaattagataccactctatttggcaaaaagtcatctccccttactgagtctggtggacctctgagcttgagtg |
| | aagaaaataatgattcaaagttgttagaatcaggtttaatgaatagccaagaaagttcatggggaaaaaatgtat |
| | cgtcaacagagagtggtaggttatttaaagggaaaagagctcatggacctgctttgttgactaaagataatgcct |
| | tattcaaagttagcatctctttgttaaagacaaacaaaacttccaataattcagcaactaatagaaagactcacatt |
| | gatggcccatcattattaattgagaatagtccatcagtctggcaaaatatattagaaagtgacactgagtttaaaa |
| | aagtgacacctttgattcatgacagaatgcttatggacaaaaatgctacagctttgaggctaaatcatatgtcaaa |
| | taaaactacttcatcaaaaaacatggaaatggtccaacagaaaaaagagggccccattccaccagatgcacaa |
| | aatccagatatgtcgttctttaagatgctattcttgccagaatcagcaaggtggatacaaaggactcatggaaag |
| | aactctctgaactctgggcaaggccccagtccaaagcaattagtatccttaggaccagaaaaatctgtggaag |
| | gtcagaatttcttgtctgagaaaaacaaagtggtagtaggaaagggtgaatttacaaaggacgtaggactcaaa |
| | gagatggtttttccaagcagcagaaacctatttcttactaacttggataatttacatgaaaataatacacacaatca |
| | agaaaaaaaaattcaggaagaaatagaaaagaaggaaacattaatccaagagaatgtagttttgcctcagata |
| | catacagtgactggcactaagaatttcatgaagaaccttttcttactgagcactaggcaaaatgtagaaggttcat |
| | atgacggggcatatgctccagtacttcaagattttaggtcattaaatgattcaacaaatagaacaaagaaacaca |
| | cagctcatttctcaaaaaaaggggaggaagaaaacttggaaggcttgggaaatcaaaccaagcaaattgtaga |
| | gaaatatgcatgcaccacaaggatatctcctaatacaagccagcagaattttgtcacgcaacgtagtaagagag |
| | ctttgaaacaattcagactcccactagaagaaacagaacttgaaaaaaggataattgtggatgacacctcaacc |
| | cagtggtccaaaaacatgaaacatttgaccccgagcaccctcacacagatagactacaatgagaaggagaaa |
| | ggggccattactcagtctcccttatcagattgccttacgaggagtcatagcatccctcaagcaaatagatctccat |
| | tacccattgcaaaggtatcatcatttccatctattagacctatatatctgaccagggtcctattccaagacaactctt |
| | ctcatcttccagcagcatcttatagaaagaaagattctggggtccaagaaagcagtcatttcttacaaggagcca |
| | aaaaaaataacctttctttagccattctaaccttggagatgactggtgatcaaagagaggttggctccctgggga |
| | caagtgccacaaattcagtcacatacaagaaagttgagaacactgttctcccgaaaccagacttgcccaaaac |
| | atctggcaaagttgaattgcttccaaaagttcacatttatcagaaggacctattccctacggaaactagcaatggg |
| | tctcctggccatctggatctcgtggaagggagccttcttcagggaacagagggagcgattaagtggaatgaag |
| | caaacagacctggaaaagttccctttctgagagtagcaacagaaagctctgcaaagactccctccaagctattg |
| | gatcctcttgcttgggataaccactatggtactcagataccaaaagaagagtggaaatcccaagagaagtcacc |
| | agaaaaaacagcttttaagaaaaaggataccattttgtccctgaacgcttgtgaaagcaatcatgcaatagcagc |
| | aataaatgagggacaaaataagcccgaaatagaagtcacctgggcaaagcaaggtaggactgaaaggctgt |
| | gctctcaaaacccaccagtcttgaaacgccatcaacgggaaataactcgtactactcttcagtcagatcaagag |
| | gaaattgactatgatgataccatatcagttgaaatgaagaaggaagattttgacatttatgatgaggatgaaaatc |
| | agagcccccgcagctttcaaaagaaaacacgacactattttattgctgcagtggagaggctctgggattatggg |
| | atgagtagctccccacatgttctaagaaacagggctcagagtggcagtgtccctcagttcaagaaagttgttttc |
| | caggaatttactgatggctcctttactcagcccttataccgtggagaactaaatgaacatttgggactcctggggc |
| | catatataagagcagaagttgaagataatatcatggtaactttcagaaatcaggcctctcgtccctattccttctatt |
| | ctagccttatttcttatgaggaagatcagaggcaaggagcagaacctagaaaaaactttgtcaagcctaatgaa |
| | accaaaacttacttttggaaagtgcaacatcatatggcacccactaaagatgagtttgactgcaaagcctgggct |
| | tatttctctgatgttgacctggaaaaagatgtgcactcaggcctgattggaccccttctggtctgccacactaaca |
| | cactgaaccctgctcatgggagacaagtgacagtacaggaatttgctctgtttttcaccatctttgatgagaccaa |
| | aagctggtacttcactgaaaatatggaaagaaactgcagggctccctgcaatatccagatggaagatcccactt |
| | ttaaagagaattatcgcttccatgcaatcaatggctacataatggatacactacctggcttagtaatggctcagga |
| | tcaaaggattcgatggtatctgctcagcatgggcagcaatgaaaacatccattctattcatttcagtggacatgtg |
| | ttcactgtacgaaaaaaagaggagtataaaatggcactgtacaatctctatccaggtgtttttgagacagtggaa |
| | atgttaccatccaaagctggaatttggcgggtggaatgccttattggcgagcatctacatgctgggatgagcac |
| | actttttctggtgtacagcaataagtgtcagactcccctgggaatggcttctggacacattagagattttcagatta |
| | cagcttcaggacaatatggacagtgggccccaaagctggccagacttcattattccggatcaatcaatgcctgg |
| | agcaccaaggagcccttttcttggatcaaggtggatctgttggcaccaatgattattcacggcatcaagaccca |
| | gggtgcccgtcagaagttctccagcctctacatctctcagtttatcatcatgtatagtcttgatgggaagaagtgg |
| | cagacttatcgaggaaattccactggaaccttaatggtcttctttggcaatgtggattcatctgggataaaacaca |
| | atatttttaaccctccaattattgctcgatacatccgtttgcacccaactcattatagcattcgcagcactcttcgcat |
| | ggagttgatgggctgtgatttaaatagttgcagcatgccattgggaatggagagtaaagcaatatcagatgcac |
| | agattactgcttcatcctactttaccaatatgtttgccacctggtctccttcaaaagctcgacttcacctccaaggga |
| | ggagtaatgcctggagacctcaggtgaataatccaaaagagtggctgcaagtggacttccagaagacaatga |
| | aagtcacaggagtaactactcagggagtaaaatctctgcttaccagcatgtatgtgaaggagttcctcatctcca |
| | gcagtcaagatggccatcagtggactctcttttttcagaatggcaaagtaaaggtttttcagggaaatcaagactc |
| | cttcacacctgtggtgaactctctagacccaccgttactgactcgctaccttcgaattcacccccagagttgggtg |
| | caccagattgccctgaggatggaggttctgggctgcgaggcacaggacctctactga (SEQ ID NO |
| | 174) |
| FVIII v5 | ATGCAGATTGAACTGTCTACCTGCTTCTTTCTGTGCCTCCTGAGGTT |
| | CTGTTTTAGTGCCACAAGGAGATATTACCTAGGAGCAGTTGAGCT |
| | GTCCTGGGATTACATGCAGAGTGATCTGGGGGAGCTGCCAGTTGA |
| | TGCTAGGTTCCCTCCAAGGGTGCCTAAGTCCTTCCCCTTTAATACC |
| | TCAGTGGTGTATAAGAAGACCCTTTTTGTAGAGTTCACTGATCACC |
| | TGTTCAATATAGCTAAACCCAGGCCACCTTGGATGGGGTTGTTGGG |
| | CCCAACAATTCAGGCAGAGGTTTATGACACAGTAGTGATAACCCT |
| | CAAGAATATGGCTAGTCATCCTGTGAGCCTGCATGCAGTAGGTGT |
| | GTCTTACTGGAAGGCTTCAGAGGGTGCTGAGTATGATGATCAGAC |
| | TTCTCAGAGAGAGAAGGAAGATGACAAAGTCTTTCCAGGGGGATC |
| | ACATACATATGTTTGGCAGGTTCTTAAAGAGAATGGTCCTATGGCC |
| | AGTGACCCCCTGTGTCTGACTTACTCCTACCTGAGCCATGTGGACC |
| | TGGTGAAGGATCTGAACTCTGGGCTGATAGGTGCACTGCTGGTGT |
| | GTAGGGAAGGTAGCCTTGCCAAAGAGAAGACCCAAACACTCCACA |
| | AGTTCATCTTGCTCTTTGCAGTTTTTGATGAAGGAAAAAGCTGGCA |
| | TAGTGAAACCAAAAACTCTTTGATGCAGGATAGGGATGCAGCTAG |
| | TGCTAGGGCCTGGCCAAAGATGCACACAGTCAATGGTTATGTGAA |
| | CAGAAGTCTTCCAGGTCTGATAGGATGCCACAGGAAAAGTGTGTA |
| | CTGGCATGTGATAGGCATGGGAACCACCCCAGAAGTGCACTCAAT |
| | ATTCCTTGAGGGTCATACCTTCCTTGTGAGAAATCATAGGCAGGCA |
| | TCCTTGGAGATCAGTCCCATCACCTTCTTAACTGCCCAAACCCTTC |
| | TGATGGACCTTGGTCAATTTCTGCTGTTTTGCCATATTTCTTCCCAT |
| | CAGCATGATGGGATGGAGGCCTATGTAAAGGTGGATTCTTGCCCA |
| | GAGGAACCCCAGCTGAGGATGAAGAACAATGAGGAAGCAGAGGA |
| | TTATGATGATGATCTTACTGATAGTGAGATGGATGTGGTTAGGTTT |
| | GATGATGACAACAGCCCCAGCTTTATCCAGATTAGGTCTGTTGCTA |
| | AGAAGCATCCAAAGACATGGGTTCATTACATAGCTGCAGAAGAAG |
| | AGGACTGGGATTATGCCCCCCTAGTCCTTGCCCCAGATGATAGATC |
| | CTATAAATCTCAGTATCTGAACAATGGACCTCAGAGGATAGGCAG |
| | GAAGTATAAGAAAGTGAGATTCATGGCATATACAGATGAGACCTT |
| | CAAGACCAGAGAAGCAATCCAACATGAAAGTGGGATTCTGGGCCC |
| | ACTGCTGTATGGTGAGGTGGGTGACACCCTGCTGATAATCTTCAAG |
| | AATCAGGCCAGCAGGCCTTATAACATCTATCCACATGGGATAACA |
| | GATGTAAGGCCTCTGTATAGTAGAAGGCTGCCCAAAGGGGTGAAA |
| | CACCTGAAAGACTTTCCTATTCTTCCAGGTGAAATCTTCAAATACA |
| | AGTGGACAGTCACAGTGGAGGATGGGCCTACTAAAAGTGACCCCA |
| | GGTGCCTGACTAGGTACTATAGTTCCTTTGTTAACATGGAAAGGGA |
| | CCTAGCTAGTGGGCTTATAGGCCCCCTTCTGATCTGTTATAAAGAA |
| | TCTGTTGATCAGAGGGGCAACCAGATCATGTCAGACAAAAGGAAT |
| | GTGATACTTTTTAGTGTGTTTGATGAGAATAGGAGCTGGTACCTGA |
| | CTGAGAACATCCAGAGGTTTTTGCCCAACCCAGCTGGTGTGCAGCT |
| | TGAGGACCCAGAGTTTCAAGCCTCAAACATCATGCACAGTATCAA |
| | TGGCTATGTTTTTGATTCTCTTCAGCTGAGTGTTTGCCTGCATGAAG |
| | TAGCTTACTGGTACATCCTCTCAATTGGGGCTCAGACAGATTTCCT |
| | TTCAGTGTTCTTCAGTGGCTACACTTTCAAGCACAAAATGGTCTAT |
| | GAGGACACTCTGACCTTATTCCCTTTTAGTGGAGAGACAGTCTTTA |
| | TGAGTATGGAGAATCCTGGGCTGTGGATTCTTGGGTGCCACAACTC |
| | TGATTTCAGAAATAGGGGAATGACTGCCCTGCTAAAGGTGAGTAG |
| | TTGTGACAAAAACACAGGGGATTACTATGAGGACAGCTATGAGGA |
| | CATCAGTGCCTATCTGCTCTCAAAGAACAATGCAATAGAGCCTAG |
| | GTCCTTCTCACAGAATAGCAGGCATCCCTCAACCAGGCAAAAACA |
| | GTTTAATGCCACCACCATACCAGAGAATGATATTGAAAAGACTGA |
| | CCCTTGGTTTGCTCACAGGACCCCAATGCCCAAAATCCAAAATGTG |
| | TCTTCAAGTGACCTGTTAATGCTGTTGAGGCAGTCACCCACCCCAC |
| | ATGGGCTGTCCCTGTCAGATCTTCAAGAGGCCAAATATGAGACCTT |
| | CAGTGATGATCCTTCACCAGGAGCCATAGATTCTAACAACTCCCTG |
| | TCAGAAATGACACACTTCAGACCTCAGTTACATCATTCTGGGGATA |
| | TGGTGTTCACACCTGAATCTGGTCTCCAGCTGAGGTTAAATGAGAA |
| | ACTTGGGACTACAGCAGCTACTGAGCTGAAAAAACTTGATTTTAA |
| | GGTGAGCTCAACAAGCAATAACCTGATTTCCACCATTCCTAGTGAC |
| | AACCTAGCTGCTGGCACTGATAACACCTCTTCTCTGGGCCCTCCTT |
| | CAATGCCAGTACATTATGATTCCCAACTTGATACCACCCTCTTTGG |
| | GAAGAAGTCTAGCCCACTTACAGAATCAGGTGGCCCACTTAGTCT |
| | CTCAGAGGAGAATAATGATTCCAAACTTTTGGAGAGTGGGCTGAT |
| | GAACTCTCAAGAGAGCAGTTGGGGGAAGAATGTATCCTCCACAGA |
| | AAGTGGGAGGTTGTTTAAGGGGAAGAGAGCTCATGGACCTGCTCT |
| | GCTGACCAAGGACAATGCTCTTTTCAAGGTTTCCATTTCCCTCCTG |
| | AAAACAAATAAGACTTCCAACAACTCAGCAACTAATAGGAAAACA |
| | CATATAGATGGACCTAGCCTGCTGATTGAAAACAGCCCCTCAGTG |
| | TGGCAGAACATATTGGAATCTGATACAGAGTTCAAGAAGGTGACC |
| | CCTCTCATCCATGATAGGATGCTGATGGATAAGAATGCCACTGCTT |
| | TGAGGCTCAATCACATGTCTAATAAAACTACCTCCAGTAAAAACA |
| | TGGAGATGGTCCAGCAGAAAAAGGAGGGTCCCATACCTCCTGATG |
| | CCCAGAATCCAGATATGTCTTTCTTTAAAATGTTGTTCCTGCCAGA |
| | ATCTGCTAGGTGGATACAAAGGACACATGGAAAAAATTCCTTGAA |
| | CTCTGGACAGGGCCCTAGTCCTAAACAGTTAGTTAGCTTGGGTCCA |
| | GAGAAGAGTGTAGAAGGGCAGAATTTCCTCAGTGAGAAGAACAA |
| | GGTGGTTGTAGGCAAGGGAGAGTTCACAAAGGATGTTGGCCTTAA |
| | GGAAATGGTATTCCCTAGCAGCAGGAACTTGTTTCTTACCAACCTG |
| | GATAATCTGCATGAAAACAACACTCACAACCAAGAGAAGAAAATT |
| | CAGGAGGAGATAGAGAAAAAGGAGACCCTCATTCAGGAAAATGT |
| | GGTGCTGCCACAGATTCACACAGTAACAGGCACAAAGAATTTCAT |
| | GAAGAATCTTTTTTTGTTGTCAACTAGGCAGAATGTGGAGGGCTCC |
| | TATGATGGGGCCTATGCACCAGTACTGCAAGATTTCAGGTCCCTCA |
| | ATGACAGCACAAATAGGACCAAAAAACATACTGCCCACTTTTCCA |
| | AAAAAGGGGAAGAGGAGAATCTGGAGGGGCTGGGGAATCAGACC |
| | AAGCAGATAGTAGAAAAATATGCTTGTACTACCAGAATCAGCCCT |
| | AACACTTCCCAGCAGAATTTTGTGACCCAGAGAAGCAAAAGGGCT |
| | CTGAAGCAGTTTAGGCTCCCCCTGGAGGAGACTGAGTTGGAAAAA |
| | AGAATCATAGTAGATGACACCTCAACCCAATGGTCTAAGAACATG |
| | AAGCACTTGACCCCTTCCACTCTCACACAGATAGATTATAATGAGA |
| | AAGAGAAGGGAGCTATCACACAATCCCCCCTCAGTGATTGTCTCA |
| | CTAGGTCACACTCTATCCCTCAGGCTAATAGAAGCCCACTCCCAAT |
| | TGCCAAGGTGTCATCATTCCCTTCTATCAGGCCTATATATTTAACA |
| | AGGGTTTTGTTCCAGGATAATTCATCTCATCTCCCAGCAGCCTCTT |
| | ATAGGAAGAAGGATTCAGGGGTCCAAGAATCCAGCCACTTTCTGC |
| | AGGGAGCTAAGAAGAACAACTTGTCCCTTGCTATATTGACCCTGG |
| | AGATGACTGGGGATCAGAGGGAGGTAGGTTCTTTGGGGACCTCAG |
| | CCACTAACTCTGTAACATACAAAAAGGTAGAAAATACAGTGTTAC |
| | CAAAACCAGATCTTCCAAAGACCTCAGGAAAAGTGGAACTGCTCC |
| | CTAAAGTGCACATTTACCAGAAGGATTTGTTTCCTACTGAGACAAG |
| | TAATGGAAGCCCTGGGCACCTAGACCTGGTGGAGGGCAGTCTTCT |
| | CCAGGGGACAGAGGGAGCCATTAAATGGAATGAGGCCAACAGAC |
| | CAGGTAAGGTTCCATTTCTGAGGGTTGCTACAGAATCCAGTGCCA |
| | AGACCCCAAGTAAACTGCTGGACCCATTGGCATGGGACAACCATT |
| | ATGGCACCCAAATTCCCAAGGAGGAATGGAAGTCTCAGGAAAAGT |
| | CACCTGAAAAGACAGCTTTCAAGAAAAAAGACACCATTTTGAGTT |
| | TGAATGCTTGTGAATCCAATCATGCCATAGCTGCTATTAATGAGGG |
| | ACAGAACAAACCTGAAATTGAGGTTACATGGGCAAAGCAGGGAA |
| | GAACTGAGAGACTGTGCTCTCAGAACCCTCCAGTTTTGAAAAGGC |
| | ACCAGAGAGAGATAACCAGAACAACCCTTCAGTCTGACCAGGAGG |
| | AGATAGATTATGATGACACTATCTCAGTTGAGATGAAAAAGGAGG |
| | ACTTTGATATCTATGATGAAGATGAAAATCAGTCTCCCAGGTCCTT |
| | CCAGAAAAAAACAAGGCATTACTTCATAGCAGCAGTAGAGAGACT |
| | GTGGGATTATGGGATGTCATCCAGTCCTCATGTCCTGAGGAACAG |
| | AGCTCAGAGTGGCTCAGTGCCTCAATTCAAGAAAGTGGTGTTCCA |
| | GGAGTTCACAGATGGCTCCTTCACACAGCCTCTCTACAGAGGAGA |
| | GCTCAATGAACACTTGGGGCTGCTGGGGCCCTATATCAGGGCAGA |
| | GGTGGAGGATAATATTATGGTGACATTTAGGAACCAAGCCTCAAG |
| | GCCATACTCATTTTATTCTTCCCTGATCTCATATGAAGAGGATCAG |
| | AGGCAGGGGGCAGAACCTAGGAAGAATTTTGTAAAGCCTAATGAG |
| | ACTAAGACTTACTTTTGGAAGGTGCAACACCACATGGCTCCCACC |
| | AAAGATGAATTTGATTGCAAGGCTTGGGCCTACTTCTCTGATGTGG |
| | ATCTGGAGAAAGATGTGCATTCTGGCTTGATAGGTCCCCTACTGGT |
| | TTGTCATACAAATACCCTGAACCCTGCACATGGCAGGCAGGTGAC |
| | TGTGCAGGAGTTTGCTCTCTTCTTCACTATATTTGATGAGACCAAG |
| | TCCTGGTACTTCACAGAGAATATGGAAAGAAACTGCAGGGCCCCA |
| | TGTAATATCCAGATGGAAGACCCCACCTTTAAGGAAAATTATAGG |
| | TTTCATGCTATTAATGGATATATAATGGACACCCTCCCAGGTCTGG |
| | TCATGGCACAGGACCAGAGGATAAGGTGGTATCTGCTTTCTATGG |
| | GCTCTAATGAAAATATTCATAGCATCCACTTTAGTGGGCATGTGTT |
| | CACTGTCAGGAAAAAAGAGGAGTATAAAATGGCCCTGTATAACCT |
| | GTACCCAGGAGTGTTTGAGACAGTAGAGATGCTTCCTAGTAAGGC |
| | TGGCATTTGGAGAGTAGAGTGCCTGATTGGAGAACATCTCCATGC |
| | TGGAATGAGCACCCTGTTCCTGGTCTATAGTAACAAGTGTCAGACC |
| | CCACTGGGCATGGCCTCAGGTCATATTAGGGACTTCCAAATCACTG |
| | CCAGTGGCCAATATGGGCAGTGGGCCCCCAAACTAGCCAGGTTGC |
| | ACTATTCTGGGAGTATTAATGCCTGGTCTACAAAGGAACCATTTTC |
| | CTGGATCAAAGTGGATCTGCTGGCCCCAATGATCATACATGGAAT |
| | TAAGACCCAGGGAGCCAGGCAGAAGTTTTCCTCCCTGTACATTAG |
| | TCAGTTCATCATTATGTACAGTTTGGATGGGAAAAAGTGGCAGAC |
| | ATATAGGGGTAACAGTACAGGAACCCTTATGGTGTTCTTTGGTAAT |
| | GTTGACAGCTCAGGAATCAAGCACAATATTTTCAACCCCCCAATC |
| | ATAGCCAGGTACATCAGGCTCCACCCTACTCATTATAGCATCAGG |
| | AGCACCCTGAGAATGGAGCTTATGGGATGTGACCTAAACTCCTGT |
| | AGTATGCCTCTGGGAATGGAATCAAAGGCCATCTCTGATGCCCAA |
| | ATTACAGCCTCATCCTACTTTACAAACATGTTTGCTACTTGGTCAC |
| | CATCCAAAGCTAGACTGCACCTGCAGGGGAGGTCAAATGCCTGGA |
| | GGCCACAAGTCAACAACCCCAAAGAATGGCTCCAAGTTGACTTTC |
| | AGAAGACTATGAAAGTGACTGGGGTGACTACTCAAGGGGTAAAGT |
| | CTCTTCTTACCAGTATGTATGTCAAAGAGTTTTTGATAAGCTCCTC |
| | CCAGGATGGCCACCAGTGGACACTGTTTTTTCAGAATGGCAAGGT |
| | GAAAGTGTTCCAAGGAAACCAGGATTCTTTTACTCCAGTGGTGAA |
| | CAGCCTGGACCCCCCCCTTCTGACCAGATACTTGAGAATCCACCCC |
| | CAGTCCTGGGTTCATCAGATTGCTCTGAGGATGGAAGTGCTGGGA |
| | TGTGAGGCCCAGGACCTCTACTAA (SEQ ID NO: 175) |
| FVIII v6 | ATGCAGATTGAACTGTCTACCTGCTTCTTTCTGTGCCTCCTGAGGTT |
| | CTGTTTTAGTGCCACAAGGAGATATTACCTAGGAGCAGTTGAGCT |
| | GTCCTGGGATTACATGCAGAGTGATCTGGGGGAGCTGCCAGTTGA |
| | TGCTAGGTTCCCTCCAAGGGTGCCTAAGTCCTTCCCCTTTAATACC |
| | TCAGTGGTGTATAAGAAGACCCTTTTTGTAGAGTTCACTGATCACC |
| | TGTTCAATATAGCTAAACCCAGGCCACCTTGGATGGGGTTGTTGGG |
| | CCCAACAATTCAGGCAGAGGTTTATGACACAGTAGTGATAACCCT |
| | CAAGAATATGGCTAGTCATCCTGTGAGCCTGCATGCAGTAGGTGT |
| | GTCTTACTGGAAGGCTTCAGAGGGTGCTGAGTATGATGATCAGAC |
| | TTCTCAGAGAGAGAAGGAAGATGACAAAGTCTTTCCAGGGGGATC |
| | ACATACATATGTTTGGCAGGTTCTTAAAGAGAATGGTCCTATGGCC |
| | AGTGACCCCCTGTGTCTGACTTACTCCTACCTGAGCCATGTGGACC |
| | TGGTGAAGGATCTGAACTCTGGGCTGATAGGTGCACTGCTGGTGT |
| | GTAGGGAAGGTAGCCTTGCCAAAGAGAAGACCCAAACACTCCACA |
| | AGTTCATCTTGCTCTTTGCAGTTTTTGATGAAGGAAAAAGCTGGCA |
| | TAGTGAAACCAAAAACTCTTTGATGCAGGATAGGGATGCAGCTAG |
| | TGCTAGGGCCTGGCCAAAGATGCACACAGTCAATGGTTATGTGAA |
| | CAGAAGTCTTCCAGGTCTGATAGGATGCCACAGGAAAAGTGTGTA |
| | CTGGCATGTGATAGGCATGGGAACCACCCCAGAAGTGCACTCAAT |
| | ATTCCTTGAGGGTCATACCTTCCTTGTGAGAAATCATAGGCAGGCA |
| | TCCTTGGAGATCAGTCCCATCACCTTCTTAACTGCCCAAACCCTTC |
| | TGATGGACCTTGGTCAATTTCTGCTGTCCTGCCATATTTCTTCCCAT |
| | CAGCATGATGGGATGGAGGCCTATGTAAAGGTGGATTCTTGCCCA |
| | GAGGAACCCCAGCTGAGGATGAAGAACAATGAGGAAGCAGAGGA |
| | TTATGATGATGATCTTACTGATAGTGAGATGGATGTGGTTAGGTTT |
| | GATGATGACAACAGCCCCAGCTTTATCCAGATTAGGTCTGTTGCTA |
| | AGAAGCATCCAAAGACATGGGTTCATTACATAGCTGCAGAAGAAG |
| | AGGACTGGGATTATGCCCCCCTAGTCCTTGCCCCAGATGATAGATC |
| | CTATAAATCTCAGTATCTGAACAATGGACCTCAGAGGATAGGCAG |
| | GAAGTATAAGAAAGTGAGATTCATGGCATATACAGATGAGACCTT |
| | CAAGACCAGAGAAGCAATCCAACATGAAAGTGGGATTCTGGGCCC |
| | ACTGCTGTATGGTGAGGTGGGTGACACCCTGCTGATAATCTTCAAG |
| | AATCAGGCCAGCAGGCCTTATAACATCTATCCACATGGGATAACA |
| | GATGTAAGGCCTCTGTATAGTAGAAGGCTGCCCAAAGGGGTGAAA |
| | CACCTGAAAGACTTTCCTATTCTTCCAGGTGAAATCTTCAAATACA |
| | AGTGGACAGTCACAGTGGAGGATGGGCCTACTAAAAGTGACCCCA |
| | GGTGCCTGACTAGGTACTATAGTTCCTTTGTTAACATGGAAAGGGA |
| | CCTAGCTAGTGGGCTTATAGGCCCCCTTCTGATCTGTTATAAAGAA |
| | TCTGTTGATCAGAGGGGCAACCAGATCATGTCAGACAAAAGGAAT |
| | GTGATACTTTTTAGTGTGTTTGATGAGAATAGGAGCTGGTACCTGA |
| | CTGAGAACATCCAGAGGTTTTTGCCCAACCCAGCTGGTGTGCAGCT |
| | TGAGGACCCAGAGTTTCAAGCCTCAAACATCATGCACAGTATCAA |
| | TGGCTATGTTTTTGATTCTCTTCAGCTGAGTGTTTGCCTGCATGAAG |
| | TAGCTTACTGGTACATCCTCTCAATTGGGGCTCAGACAGATTTCCT |
| | TTCAGTGTTCTTCAGTGGCTACACTTTCAAGCACAAAATGGTCTAT |
| | GAGGACACTCTGACCTTATTCCCTTTTAGTGGAGAGACAGTCTTTA |
| | TGAGTATGGAGAATCCTGGGCTGTGGATTCTTGGGTGCCACAACTC |
| | TGATTTCAGAAATAGGGGAATGACTGCCCTGCTAAAGGTGAGTAG |
| | TTGTGACAAAAACACAGGGGATTACTATGAGGACAGCTATGAGGA |
| | CATCAGTGCCTATCTGCTCTCAAAGAACAATGCAATAGAGCCTAG |
| | GTCCTTCTCACAGAATAGCAGGCATCCCTCAACCAGGCAAAAACA |
| | GTTTAATGCCACCACCATACCAGAGAATGATATTGAAAAGACTGA |
| | CCCTTGGTTTGCTCACAGGACCCCAATGCCCAAAATCCAAAATGTG |
| | TCTTCAAGTGACCTGTTAATGCTGTTGAGGCAGTCACCCACCCCAC |
| | ATGGGCTGTCCCTGTCAGATCTTCAAGAGGCCAAATATGAGACCTT |
| | CAGTGATGATCCTTCACCAGGAGCCATAGATTCTAACAACTCCCTG |
| | TCAGAAATGACACACTTCAGACCTCAGTTACATCATTCTGGGGATA |
| | TGGTGTTCACACCTGAATCTGGTCTCCAGCTGAGGTTAAATGAGAA |
| | ACTTGGGACTACAGCAGCTACTGAGCTGAAAAAACTTGATTTTAA |
| | GGTGAGCTCAACAAGCAATAACCTGATTTCCACCATTCCTAGTGAC |
| | AACCTAGCTGCTGGCACTGATAACACCTCTTCTCTGGGCCCTCCTT |
| | CAATGCCAGTACATTATGATTCCCAACTTGATACCACCCTCTTTGG |
| | GAAGAAGTCTAGCCCACTTACAGAATCAGGTGGCCCACTTAGTCT |
| | CTCAGAGGAGAATAATGATTCCAAACTTTTGGAGAGTGGGCTGAT |
| | GAACTCTCAAGAGAGCAGTTGGGGGAAGAATGTATCCTCCACAGA |
| | AAGTGGGAGGTTGTTTAAGGGGAAGAGAGCTCATGGACCTGCTCT |
| | GCTGACCAAGGACAATGCTCTTTTCAAGGTTTCCATTTCCCTCCTG |
| | AAAACAAATAAGACTTCCAACAACTCAGCAACTAATAGGAAAACA |
| | CATATAGATGGACCTAGCCTGCTGATTGAAAACAGCCCCTCAGTG |
| | TGGCAGAACATATTGGAATCTGATACAGAGTTCAAGAAGGTGACC |
| | CCTCTCATCCATGATAGGATGCTGATGGATAAGAATGCCACTGCTT |
| | TGAGGCTCAATCACATGTCTAATAAAACTACCTCCAGTAAAAACA |
| | TGGAGATGGTCCAGCAGAAAAAGGAGGGTCCCATACCTCCTGATG |
| | CCCAGAATCCAGATATGTCTTTCTTTAAAATGTTGTTCCTGCCAGA |
| | ATCTGCTAGGTGGATACAAAGGACACATGGAAAAAATTCCTTGAA |
| | CTCTGGACAGGGCCCTAGTCCTAAACAGTTAGTTAGCTTGGGTCCA |
| | GAGAAGAGTGTAGAAGGGCAGAATTTCCTCAGTGAGAAGAACAA |
| | GGTGGTTGTAGGCAAGGGAGAGTTCACAAAGGATGTTGGCCTTAA |
| | GGAAATGGTATTCCCTAGCAGCAGGAACTTGTTTCTTACCAACCTG |
| | GATAATCTGCATGAAAACAACACTCACAACCAAGAGAAGAAAATT |
| | CAGGAGGAGATAGAGAAAAAGGAGACCCTCATTCAGGAAAATGT |
| | GGTGCTGCCACAGATTCACACAGTAACAGGCACAAAGAATTTCAT |
| | GAAGAATCTTTTTTTGTTGTCAACTAGGCAGAATGTGGAGGGCTCC |
| | TATGATGGGGCCTATGCACCAGTACTGCAAGATTTCAGGTCCCTCA |
| | ATGACAGCACAAATAGGACCAAAAAACATACTGCCCACTTTTCCA |
| | AAAAAGGGGAAGAGGAGAATCTGGAGGGGCTGGGGAATCAGACC |
| | AAGCAGATAGTAGAAAAATATGCTTGTACTACCAGAATCAGCCCT |
| | AACACTTCCCAGCAGAATTTTGTGACCCAGAGAAGCAAAAGGGCT |
| | CTGAAGCAGTTTAGGCTCCCCCTGGAGGAGACTGAGTTGGAAAAA |
| | AGAATCATAGTAGATGACACCTCAACCCAATGGTCTAAGAACATG |
| | AAGCACTTGACCCCTTCCACTCTCACACAGATAGATTATAATGAGA |
| | AAGAGAAGGGAGCTATCACACAATCCCCCCTCAGTGATTGTCTCA |
| | CTAGGTCACACTCTATCCCTCAGGCTAATAGAAGCCCACTCCCAAT |
| | TGCCAAGGTGTCATCATTCCCTTCTATCAGGCCTATATATTTAACA |
| | AGGGTTTTGTTCCAGGATAATTCATCTCATCTCCCAGCAGCCTCTT |
| | ATAGGAAGAAGGATTCAGGGGTCCAAGAATCCAGCCACTTTCTGC |
| | AGGGAGCTAAGAAGAACAACTTGTCCCTTGCTATATTGACCCTGG |
| | AGATGACTGGGGATCAGAGGGAGGTAGGTTCTTTGGGGACCTCAG |
| | CCACTAACTCTGTAACATACAAAAAGGTAGAAAATACAGTGTTAC |
| | CAAAACCAGATCTTCCAAAGACCTCAGGAAAAGTGGAACTGCTCC |
| | CTAAAGTGCACATTTACCAGAAGGATTTGTTTCCTACTGAGACAAG |
| | TAATGGAAGCCCTGGGCACCTAGACCTGGTGGAGGGCAGTCTTCT |
| | CCAGGGGACAGAGGGAGCCATTAAATGGAATGAGGCCAACAGAC |
| | CAGGTAAGGTTCCATTTCTGAGGGTTGCTACAGAATCCAGTGCCA |
| | AGACCCCAAGTAAACTGCTGGACCCATTGGCATGGGACAACCATT |
| | ATGGCACCCAAATTCCCAAGGAGGAATGGAAGTCTCAGGAAAAGT |
| | CACCTGAAAAGACAGCTTTCAAGAAAAAAGACACCATTTTGAGTT |
| | TGAATGCTTGTGAATCCAATCATGCCATAGCTGCTATTAATGAGGG |
| | ACAGAACAAACCTGAAATTGAGGTTACATGGGCAAAGCAGGGAA |
| | GAACTGAGAGACTGTGCTCTCAGAACCCTCCAGTTTTGAAAAGGC |
| | ACCAGAGAGAGATAACCAGAACAACCCTTCAGTCTGACCAGGAGG |
| | AGATAGATTATGATGACACTATCTCAGTTGAGATGAAAAAGGAGG |
| | ACTTTGATATCTATGATGAAGATGAAAATCAGTCTCCCAGGTCCTT |
| | CCAGAAAAAAACAAGGCATTACTTCATAGCAGCAGTAGAGAGACT |
| | GTGGGATTATGGGATGTCATCCAGTCCTCATGTCCTGAGGAACAG |
| | AGCTCAGAGTGGCTCAGTGCCTCAATTCAAGAAAGTGGTGTTCCA |
| | GGAGTTCACAGATGGCTCCTTCACACAGCCTCTCTACAGAGGAGA |
| | GCTCAATGAACACTTGGGGCTGCTGGGGCCCTATATCAGGGCAGA |
| | GGTGGAGGATAATATTATGGTGACATTTAGGAACCAAGCCTCAAG |
| | GCCATACTCATTTTATTCTTCCCTGATCTCATATGAAGAGGATCAG |
| | AGGCAGGGGGCAGAACCTAGGAAGAATTTTGTAAAGCCTAATGAG |
| | ACTAAGACTTACTTTTGGAAGGTGCAACACCACATGGCTCCCACC |
| | AAAGATGAATTTGATTGCAAGGCTTGGGCCTACTTCTCTGATGTGG |
| | ATCTGGAGAAAGATGTGCATTCTGGCTTGATAGGTCCCCTACTGGT |
| | TTGTCATACAAATACCCTGAACCCTGCACATGGCAGGCAGGTGAC |
| | TGTGCAGGAGTTTGCTCTCTTCTTCACTATATTTGATGAGACCAAG |
| | TCCTGGTACTTCACAGAGAATATGGAAAGAAACTGCAGGGCCCCA |
| | TGTAATATCCAGATGGAAGACCCCACCTTTAAGGAAAATTATAGG |
| | TTTCATGCTATTAATGGATATATAATGGACACCCTCCCAGGTCTGG |
| | TCATGGCACAGGACCAGAGGATAAGGTGGTATCTGCTTTCTATGG |
| | GCTCTAATGAAAATATTCATAGCATCCACTTTAGTGGGCATGTGTT |
| | CACTGTCAGGAAAAAAGAGGAGTATAAAATGGCCCTGTATAACCT |
| | GTACCCAGGAGTGTTTGAGACAGTAGAGATGCTTCCTAGTAAGGC |
| | TGGCATTTGGAGAGTAGAGTGCCTGATTGGAGAACATCTCCATGC |
| | TGGAATGAGCACCCTGTTCCTGGTCTATAGTAACAAGTGTCAGACC |
| | CCACTGGGCATGGCCTCAGGTCATATTAGGGACTTCCAAATCACTG |
| | CCAGTGGCCAATATGGGCAGTGGGCCCCCAAACTAGCCAGGTTGC |
| | ACTATTCTGGGAGTATTAATGCCTGGTCTACAAAGGAACCATTTTC |
| | CTGGATCAAAGTGGATCTGCTGGCCCCAATGATCATACATGGAAT |
| | TAAGACCCAGGGAGCCAGGCAGAAGTTTTCCTCCCTGTACATTAG |
| | TCAGTTCATCATTATGTACAGTTTGGATGGGAAAAAGTGGCAGAC |
| | ATATAGGGGTAACAGTACAGGAACCCTTATGGTGTTCTTTGGTAAT |
| | GTTGACAGCTCAGGAATCAAGCACAATATTTTCAACCCCCCAATC |
| | ATAGCCAGGTACATCAGGCTCCACCCTACTCATTATAGCATCAGG |
| | AGCACCCTGAGAATGGAGCTTATGGGATGTGACCTAAACTCCTGT |
| | AGTATGCCTCTGGGAATGGAATCAAAGGCCATCTCTGATGCCCAA |
| | ATTACAGCCTCATCCTACTTTACAAACATGTTTGCTACTTGGTCAC |
| | CATCCAAAGCTAGACTGCACCTGCAGGGGAGGTCAAATGCCTGGA |
| | GGCCACAAGTCAACAACCCCAAAGAATGGCTCCAAGTTGACTTTC |
| | AGAAGACTATGAAAGTGACTGGGGTGACTACTCAAGGGGTAAAGT |
| | CTCTTCTTACCAGTATGTATGTCAAAGAGTTTTTGATAAGCTCCTC |
| | CCAGGATGGCCACCAGTGGACACTGTTTTTTCAGAATGGCAAGGT |
| | GAAAGTGTTCCAAGGAAACCAGGATTCTTTTACTCCAGTGGTGAA |
| | CAGCCTGGACCCTCCCCTTCTGACCAGATACTTGAGAATCCACCCC |
| | CAGTCCTGGGTTCATCAGATTGCTCTGAGGATGGAAGTGCTGGGA |
| | TGTGAGGCCCAGGACCTCTACTAA (SEQ ID NO: 177) |
| FVIII v7 | ATGCAGATTGAACTGTCTACCTGCTTCTTTCTGTGCCTCCTGAGGTT |
| | CTGTTTTAGTGCCACAAGGAGATATTACCTAGGAGCAGTTGAGCT |
| | GTCCTGGGATTACATGCAGAGTGATCTGGGGGAGCTGCCAGTTGA |
| | TGCTAGGTTCCCTCCAAGGGTGCCTAAGTCCTTCCCCTTTAATACC |
| | TCAGTGGTGTATAAGAAGACCCTTTTTGTAGAGTTCACTGATCACC |
| | TGTTCAATATAGCTAAACCCAGGCCACCTTGGATGGGGTTGTTGGG |
| | CCCAACAATTCAGGCAGAGGTTTATGACACAGTAGTGATAACCCT |
| | CAAGAATATGGCTAGTCATCCTGTGAGCCTGCATGCAGTAGGTGT |
| | GTCTTACTGGAAGGCTTCAGAGGGTGCTGAGTATGATGATCAGAC |
| | TTCTCAGAGAGAGAAGGAAGATGACAAAGTCTTTCCAGGGGGATC |
| | ACATACATATGTTTGGCAGGTTCTTAAAGAGAATGGTCCTATGGCC |
| | AGTGACCCCCTGTGTCTGACTTACTCCTACCTGAGCCATGTGGACC |
| | TGGTGAAGGATCTGAACTCTGGGCTGATAGGTGCACTGCTGGTGT |
| | GTAGGGAAGGTAGCCTTGCCAAAGAGAAGACCCAAACACTCCACA |
| | AGTTCATCTTGCTCTTTGCAGTTTTTGATGAAGGAAAAAGCTGGCA |
| | TAGTGAAACCAAAAACTCTTTGATGCAGGATAGGGATGCAGCTAG |
| | TGCTAGGGCCTGGCCAAAGATGCACACAGTCAATGGTTATGTGAA |
| | CAGAAGTCTTCCAGGTCTGATAGGATGCCACAGGAAAAGTGTGTA |
| | CTGGCATGTGATAGGCATGGGAACCACCCCAGAAGTGCACTCAAT |
| | ATTCCTTGAGGGTCATACCTTCCTTGTGAGAAATCATAGGCAGGCA |
| | TCCTTGGAGATCAGTCCCATCACCTTCTTAACTGCCCAAACCCTTC |
| | TGATGGACCTTGGTCAATTTCTGCTGTCCTGCCATATTTCTTCCCAT |
| | CAGCATGATGGGATGGAGGCCTATGTAAAGGTGGATTCTTGCCCA |
| | GAGGAACCCCAGCTGAGGATGAAGAACAATGAGGAAGCAGAGGA |
| | TTATGATGATGATCTTACTGATAGTGAGATGGATGTGGTTAGGTTT |
| | GATGATGACAACAGCCCCAGCTTTATCCAGATTAGGTCTGTTGCTA |
| | AGAAGCATCCAAAGACATGGGTTCATTACATAGCTGCAGAAGAAG |
| | AGGACTGGGATTATGCCCCCCTAGTCCTTGCCCCAGATGATAGATC |
| | CTATAAATCTCAGTATCTGAACAATGGACCTCAGAGGATAGGCAG |
| | GAAGTATAAGAAAGTGAGATTCATGGCATATACAGATGAGACCTT |
| | CAAGACCAGAGAAGCAATCCAACATGAAAGTGGGATTCTGGGCCC |
| | ACTGCTGTATGGTGAGGTGGGTGACACCCTGCTGATAATCTTCAAG |
| | AATCAGGCCAGCAGGCCTTATAACATCTATCCACATGGGATAACA |
| | GATGTAAGGCCTCTGTATAGTAGAAGGCTGCCCAAAGGGGTGAAA |
| | CACCTGAAAGACTTTCCTATTCTTCCAGGTGAAATCTTCAAATACA |
| | AGTGGACAGTCACAGTGGAGGATGGGCCTACTAAAAGTGACCCCA |
| | GGTGCCTGACTAGGTACTATAGTTCCTTTGTTAACATGGAAAGGGA |
| | CCTAGCTAGTGGGCTTATAGGCCCCCTTCTGATCTGTTATAAAGAA |
| | TCTGTTGATCAGAGGGGCAACCAGATCATGTCAGACAAAAGGAAT |
| | GTGATACTTTTTAGTGTGTTTGATGAGAATAGGAGCTGGTACCTGA |
| | CTGAGAACATCCAGAGGTTTTTGCCCAACCCAGCTGGTGTGCAGCT |
| | TGAGGACCCAGAGTTTCAAGCCTCAAACATCATGCACAGTATCAA |
| | TGGCTATGTTTTTGATTCTCTTCAGCTGAGTGTTTGCCTGCATGAAG |
| | TAGCTTACTGGTACATCCTCTCAATTGGGGCTCAGACAGATTTCCT |
| | TTCAGTGTTCTTCAGTGGCTACACTTTCAAGCACAAAATGGTCTAT |
| | GAGGACACTCTGACCTTATTCCCTTTTAGTGGAGAGACAGTCTTTA |
| | TGAGTATGGAGAATCCTGGGCTGTGGATTCTTGGGTGCCACAACTC |
| | TGATTTCAGAAATAGGGGAATGACTGCCCTGCTAAAGGTGAGTAG |
| | TTGTGACAAAAACACAGGGGATTACTATGAGGACAGCTATGAGGA |
| | CATCAGTGCCTATCTGCTCTCAAAGAACAATGCAATAGAGCCTAG |
| | GTCCTTCTCACAGAATCCTCCAGTTTTGAAACACCACCAGAGAGA |
| | GATAACCAGAACAACCCTTCAGTCTGACCAGGAGGAGATAGATTA |
| | TGATGACACTATCTCAGTTGAGATGAAAAAGGAGGACTTTGATAT |
| | CTATGATGAAGATGAAAATCAGTCTCCCAGGTCCTTCCAGAAAAA |
| | AACAAGGCATTACTTCATAGCAGCAGTAGAGAGACTGTGGGATTA |
| | TGGGATGTCATCCAGTCCTCATGTCCTGAGGAACAGAGCTCAGAG |
| | TGGCTCAGTGCCTCAATTCAAGAAAGTGGTGTTCCAGGAGTTCAC |
| | AGATGGCTCCTTCACACAGCCTCTCTACAGAGGAGAGCTCAATGA |
| | ACACTTGGGGCTGCTGGGGCCCTATATCAGGGCAGAGGTGGAGGA |
| | TAATATTATGGTGACATTTAGGAACCAAGCCTCAAGGCCATACTC |
| | ATTTTATTCTTCCCTGATCTCATATGAAGAGGATCAGAGGCAGGGG |
| | GCAGAACCTAGGAAGAATTTTGTAAAGCCTAATGAGACTAAGACT |
| | TACTTTTGGAAGGTGCAACACCACATGGCTCCCACCAAAGATGAA |
| | TTTGATTGCAAGGCTTGGGCCTACTTCTCTGATGTGGATCTGGAGA |
| | AAGATGTGCATTCTGGCTTGATAGGTCCCCTACTGGTTTGTCATAC |
| | AAATACCCTGAACCCTGCACATGGCAGGCAGGTGACTGTGCAGGA |
| | GTTTGCTCTCTTCTTCACTATATTTGATGAGACCAAGTCCTGGTACT |
| | TCACAGAGAATATGGAAAGAAACTGCAGGGCCCCATGTAATATCC |
| | AGATGGAAGACCCCACCTTTAAGGAAAATTATAGGTTTCATGCTA |
| | TTAATGGATATATAATGGACACCCTCCCAGGTCTGGTCATGGCACA |
| | GGACCAGAGGATAAGGTGGTATCTGCTTTCTATGGGCTCTAATGA |
| | AAATATTCATAGCATCCACTTTAGTGGGCATGTGTTCACTGTCAGG |
| | AAAAAAGAGGAGTATAAAATGGCCCTGTATAACCTGTACCCAGGA |
| | GTGTTTGAGACAGTAGAGATGCTTCCTAGTAAGGCTGGCATTTGG |
| | AGAGTAGAGTGCCTGATTGGAGAACATCTCCATGCTGGAATGAGC |
| | ACCCTGTTCCTGGTCTATAGTAACAAGTGTCAGACCCCACTGGGCA |
| | TGGCCTCAGGTCATATTAGGGACTTCCAAATCACTGCCAGTGGCCA |
| | ATATGGGCAGTGGGCCCCCAAACTAGCCAGGTTGCACTATTCTGG |
| | GAGTATTAATGCCTGGTCTACAAAGGAACCATTTTCCTGGATCAAA |
| | GTGGATCTGCTGGCCCCAATGATCATACATGGAATTAAGACCCAG |
| | GGAGCCAGGCAGAAGTTTTCCTCCCTGTACATTAGTCAGTTCATCA |
| | TTATGTACAGTTTGGATGGGAAAAAGTGGCAGACATATAGGGGTA |
| | ACAGTACAGGAACCCTTATGGTGTTCTTTGGTAATGTTGACAGCTC |
| | AGGAATCAAGCACAATATTTTCAACCCCCCAATCATAGCCAGGTA |
| | CATCAGGCTCCACCCTACTCATTATAGCATCAGGAGCACCCTGAG |
| | AATGGAGCTTATGGGATGTGACCTAAACTCCTGTAGTATGCCTCTG |
| | GGAATGGAATCAAAGGCCATCTCTGATGCCCAAATTACAGCCTCA |
| | TCCTACTTTACAAACATGTTTGCTACTTGGTCACCATCCAAAGCTA |
| | GACTGCACCTGCAGGGGAGGTCAAATGCCTGGAGGCCACAAGTCA |
| | ACAACCCCAAAGAATGGCTCCAAGTTGACTTTCAGAAGACTATGA |
| | AAGTGACTGGGGTGACTACTCAAGGGGTAAAGTCTCTTCTTACCA |
| | GTATGTATGTCAAAGAGTTTTTGATAAGCTCCTCCCAGGATGGCCA |
| | CCAGTGGACACTGTTTTTTCAGAATGGCAAGGTGAAAGTGTTCCA |
| | AGGAAACCAGGATTCTTTTACTCCAGTGGTGAACAGCCTGGACCC |
| | TCCTCTTCTGACCAGATACTTGAGAATCCACCCCCAGTCCTGGGTT |
| | CATCAGATTGCTCTGAGGATGGAAGTGCTGGGATGTGAGGCCCAG |
| | GACCTCTACTAA (SEQ ID NO: 178) |
| FVIII v8 | ATGCAGATTGAACTGTCTACCTGCTTCTTTCTGTGCCTCCTGAGGTT |
| | CTGTTTTAGTGCCACAAGGAGATATTACCTAGGAGCAGTTGAGCT |
| | GTCCTGGGATTACATGCAGAGTGATCTGGGGGAGCTGCCAGTTGA |
| | TGCTAGGTTCCCTCCAAGGGTGCCTAAGTCCTTCCCCTTTAATACC |
| | TCAGTGGTGTATAAGAAGACCCTTTTTGTAGAGTTCACTGATCACC |
| | TGTTCAATATAGCTAAACCCAGGCCACCTTGGATGGGGTTGTTGGG |
| | CCCAACAATTCAGGCAGAGGTTTATGACACAGTAGTGATAACCCT |
| | CAAGAATATGGCTAGTCATCCTGTGAGCCTGCATGCAGTAGGTGT |
| | GTCTTACTGGAAGGCTTCAGAGGGTGCTGAGTATGATGATCAGAC |
| | TTCTCAGAGAGAGAAGGAAGATGACAAAGTCTTTCCAGGGGGATC |
| | ACATACATATGTTTGGCAGGTTCTTAAAGAGAATGGTCCTATGGCC |
| | AGTGACCCCCTGTGTCTGACTTACTCCTACCTGAGCCATGTGGACC |
| | TGGTGAAGGATCTGAACTCTGGGCTGATAGGTGCACTGCTGGTGT |
| | GTAGGGAAGGTAGCCTTGCCAAAGAGAAGACCCAAACACTCCACA |
| | AGTTCATCTTGCTCTTTGCAGTTTTTGATGAAGGAAAAAGCTGGCA |
| | TAGTGAAACCAAAAACTCTTTGATGCAGGATAGGGATGCAGCTAG |
| | TGCTAGGGCCTGGCCAAAGATGCACACAGTCAATGGTTATGTGAA |
| | CAGAAGTCTTCCAGGTCTGATAGGATGCCACAGGAAAAGTGTGTA |
| | CTGGCATGTGATAGGCATGGGAACCACCCCAGAAGTGCACTCAAT |
| | ATTCCTTGAGGGTCATACCTTCCTTGTGAGAAATCATAGGCAGGCA |
| | TCCTTGGAGATCAGTCCCATCACCTTCTTAACTGCCCAAACCCTTC |
| | TGATGGACCTTGGTCAATTTCTGCTGTCCTGCCATATTTCTTCCCAT |
| | CAGCATGATGGGATGGAGGCCTATGTAAAGGTGGATTCTTGCCCA |
| | GAGGAACCCCAGCTGAGGATGAAGAACAATGAGGAAGCAGAGGA |
| | TTATGATGATGATCTTACTGATAGTGAGATGGATGTGGTTAGGTTT |
| | GATGATGACAACAGCCCCAGCTTTATCCAGATTAGGTCTGTTGCTA |
| | AGAAGCATCCAAAGACATGGGTTCATTACATAGCTGCAGAAGAAG |
| | AGGACTGGGATTATGCCCCCCTAGTCCTTGCCCCAGATGATAGATC |
| | CTATAAATCTCAGTATCTGAACAATGGACCTCAGAGGATAGGCAG |
| | GAAGTATAAGAAAGTGAGATTCATGGCATATACAGATGAGACCTT |
| | CAAGACCAGAGAAGCAATCCAACATGAAAGTGGGATTCTGGGCCC |
| | ACTGCTGTATGGTGAGGTGGGTGACACCCTGCTGATAATCTTCAAG |
| | AATCAGGCCAGCAGGCCTTATAACATCTATCCACATGGGATAACA |
| | GATGTAAGGCCTCTGTATAGTAGAAGGCTGCCCAAAGGGGTGAAA |
| | CACCTGAAAGACTTTCCTATTCTTCCAGGTGAAATCTTCAAATACA |
| | AGTGGACAGTCACAGTGGAGGATGGGCCTACTAAAAGTGACCCCA |
| | GGTGCCTGACTAGGTACTATAGTTCCTTTGTTAACATGGAAAGGGA |
| | CCTAGCTAGTGGGCTTATAGGCCCCCTTCTGATCTGTTATAAAGAA |
| | TCTGTTGATCAGAGGGGCAACCAGATCATGTCAGACAAAAGGAAT |
| | GTGATACTTTTTAGTGTGTTTGATGAGAATAGGAGCTGGTACCTGA |
| | CTGAGAACATCCAGAGGTTTTTGCCCAACCCAGCTGGTGTGCAGCT |
| | TGAGGACCCAGAGTTTCAAGCCTCAAACATCATGCACAGTATCAA |
| | TGGCTATGTTTTTGATTCTCTTCAGCTGAGTGTTTGCCTGCATGAAG |
| | TAGCTTACTGGTACATCCTCTCAATTGGGGCTCAGACAGATTTCCT |
| | TTCAGTGTTCTTCAGTGGCTACACTTTCAAGCACAAAATGGTCTAT |
| | GAGGACACTCTGACCTTATTCCCTTTTAGTGGAGAGACAGTCTTTA |
| | TGAGTATGGAGAATCCTGGGCTGTGGATTCTTGGGTGCCACAACTC |
| | TGATTTCAGAAATAGGGGAATGACTGCCCTGCTAAAGGTGAGTAG |
| | TTGTGACAAAAACACAGGGGATTACTATGAGGACAGCTATGAGGA |
| | CATCAGTGCCTATCTGCTCTCAAAGAACAATGCAATAGAGCCTAG |
| | GTCCTTCTCACAGAATAGCAGGCATCCCTCAACCAGGCAAAAACA |
| | GTTTAATGCCACCACCATACCAGAGAATGATATTGAAAAGACTGA |
| | CCCTTGGTTTGCTCACAGGACCCCAATGCCCAAAATCCAAAATGTG |
| | TCTTCAAGTGACCTGTTAATGCTGTTGAGGCAGTCACCCACCCCAC |
| | ATGGGCTGTCCCTGTCAGATCTTCAAGAGGCCAAATATGAGACCTT |
| | CAGTGATGATCCTTCACCAGGAGCCATAGATTCTAACAACTCCCTG |
| | TCAGAAATGACACACTTCAGACCTCAGTTACATCATTCTGGGGATA |
| | TGGTGTTCACACCTGAATCTGGTCTCCAGCTGAGGTTAAATGAGAA |
| | ACTTGGGACTACAGCAGCTACTGAGCTGAAAAAACTTGATTTTAA |
| | GGTGAGCTCAACAAGCAATAACCTGATTTCCACCATTCCTAGTGAC |
| | AACCTAGCTGCTGGCACTGATAACACCTCTTCTCTGGGCCCTCCTT |
| | CAATGCCAGTACATTATGATTCCCAACTTGATACCACCCTCTTTGG |
| | GAAGAAGTCTAGCCCACTTACAGAATCAGGTGGCCCACTTAGTCT |
| | CTCAGAGGAGAATAATGATTCCAAACTTTTGGAGAGTGGGCTGAT |
| | GAACTCTCAAGAGAGCAGTTGGGGGAAGAATGTATCCAGGTCCTT |
| | CCAGAAAAAAACAAGGCATTACTTCATAGCAGCAGTAGAGAGACT |
| | GTGGGATTATGGGATGTCATCCAGTCCTCATGTCCTGAGGAACAG |
| | AGCTCAGAGTGGCTCAGTGCCTCAATTCAAGAAAGTGGTGTTCCA |
| | GGAGTTCACAGATGGCTCCTTCACACAGCCTCTCTACAGAGGAGA |
| | GCTCAATGAACACTTGGGGCTGCTGGGGCCCTATATCAGGGCAGA |
| | GGTGGAGGATAATATTATGGTGACATTTAGGAACCAAGCCTCAAG |
| | GCCATACTCATTTTATTCTTCCCTGATCTCATATGAAGAGGATCAG |
| | AGGCAGGGGGCAGAACCTAGGAAGAATTTTGTAAAGCCTAATGAG |
| | ACTAAGACTTACTTTTGGAAGGTGCAACACCACATGGCTCCCACC |
| | AAAGATGAATTTGATTGCAAGGCTTGGGCCTACTTCTCTGATGTGG |
| | ATCTGGAGAAAGATGTGCATTCTGGCTTGATAGGTCCCCTACTGGT |
| | TTGTCATACAAATACCCTGAACCCTGCACATGGCAGGCAGGTGAC |
| | TGTGCAGGAGTTTGCTCTCTTCTTCACTATATTTGATGAGACCAAG |
| | TCCTGGTACTTCACAGAGAATATGGAAAGAAACTGCAGGGCCCCA |
| | TGTAATATCCAGATGGAAGACCCCACCTTTAAGGAAAATTATAGG |
| | TTTCATGCTATTAATGGATATATAATGGACACCCTCCCAGGTCTGG |
| | TCATGGCACAGGACCAGAGGATAAGGTGGTATCTGCTTTCTATGG |
| | GCTCTAATGAAAATATTCATAGCATCCACTTTAGTGGGCATGTGTT |
| | CACTGTCAGGAAAAAAGAGGAGTATAAAATGGCCCTGTATAACCT |
| | GTACCCAGGAGTGTTTGAGACAGTAGAGATGCTTCCTAGTAAGGC |
| | TGGCATTTGGAGAGTAGAGTGCCTGATTGGAGAACATCTCCATGC |
| | TGGAATGAGCACCCTGTTCCTGGTCTATAGTAACAAGTGTCAGACC |
| | CCACTGGGCATGGCCTCAGGTCATATTAGGGACTTCCAAATCACTG |
| | CCAGTGGCCAATATGGGCAGTGGGCCCCCAAACTAGCCAGGTTGC |
| | ACTATTCTGGGAGTATTAATGCCTGGTCTACAAAGGAACCATTTTC |
| | CTGGATCAAAGTGGATCTGCTGGCCCCAATGATCATACATGGAAT |
| | TAAGACCCAGGGAGCCAGGCAGAAGTTTTCCTCCCTGTACATTAG |
| | TCAGTTCATCATTATGTACAGTTTGGATGGGAAAAAGTGGCAGAC |
| | ATATAGGGGTAACAGTACAGGAACCCTTATGGTGTTCTTTGGTAAT |
| | GTTGACAGCTCAGGAATCAAGCACAATATTTTCAACCCCCCAATC |
| | ATAGCCAGGTACATCAGGCTCCACCCTACTCATTATAGCATCAGG |
| | AGCACCCTGAGAATGGAGCTTATGGGATGTGACCTAAACTCCTGT |
| | AGTATGCCTCTGGGAATGGAATCAAAGGCCATCTCTGATGCCCAA |
| | ATTACAGCCTCATCCTACTTTACAAACATGTTTGCTACTTGGTCAC |
| | CATCCAAAGCTAGACTGCACCTGCAGGGGAGGTCAAATGCCTGGA |
| | GGCCACAAGTCAACAACCCCAAAGAATGGCTCCAAGTTGACTTTC |
| | AGAAGACTATGAAAGTGACTGGGGTGACTACTCAAGGGGTAAAGT |
| | CTCTTCTTACCAGTATGTATGTCAAAGAGTTTTTGATAAGCTCCTC |
| | CCAGGATGGCCACCAGTGGACACTGTTTTTTCAGAATGGCAAGGT |
| | GAAAGTGTTCCAAGGAAACCAGGATTCTTTTACTCCAGTGGTGAA |
| | CAGCCTGGACCCTCCTCTTCTGACCAGATACTTGAGAATCCACCCC |
| | CAGTCCTGGGTTCATCAGATTGCTCTGAGGATGGAAGTGCTGGGA |
| | TGTGAGGCCCAGGACCTCTACTAA (SEQ ID NO: 179) |
| FVIII v9 | ATGCAGATTGAACTGTCTACCTGCTTCTTTCTGTGCCTCCTGAGGTT |
| | CTGTTTTAGTGCCACAAGGAGATATTACCTAGGAGCAGTTGAGCT |
| | GTCCTGGGATTACATGCAGAGTGATCTGGGGGAGCTGCCAGTTGA |
| | TGCTAGGTTCCCTCCAAGGGTGCCTAAGTCCTTCCCCTTTAATACC |
| | TCAGTGGTGTATAAGAAGACCCTTTTTGTAGAGTTCACTGATCACC |
| | TGTTCAATATAGCTAAACCCAGGCCACCTTGGATGGGGTTGTTGGG |
| | CCCAACAATTCAGGCAGAGGTTTATGACACAGTAGTGATAACCCT |
| | CAAGAATATGGCTAGTCATCCTGTGAGCCTGCATGCAGTAGGTGT |
| | GTCTTACTGGAAGGCTTCAGAGGGTGCTGAGTATGATGATCAGAC |
| | TTCTCAGAGAGAGAAGGAAGATGACAAAGTCTTTCCAGGGGGATC |
| | ACATACATATGTTTGGCAGGTTCTTAAAGAGAATGGTCCTATGGCC |
| | AGTGACCCCCTGTGTCTGACTTACTCCTACCTGAGCCATGTGGACC |
| | TGGTGAAGGATCTGAACTCTGGGCTGATAGGTGCACTGCTGGTGT |
| | GTAGGGAAGGTAGCCTTGCCAAAGAGAAGACCCAAACACTCCACA |
| | AGTTCATCTTGCTCTTTGCAGTTTTTGATGAAGGAAAAAGCTGGCA |
| | TAGTGAAACCAAAAACTCTTTGATGCAGGATAGGGATGCAGCTAG |
| | TGCTAGGGCCTGGCCAAAGATGCACACAGTCAATGGTTATGTGAA |
| | CAGAAGTCTTCCAGGTCTGATAGGATGCCACAGGAAAAGTGTGTA |
| | CTGGCATGTGATAGGCATGGGAACCACCCCAGAAGTGCACTCAAT |
| | ATTCCTTGAGGGTCATACCTTCCTTGTGAGAAATCATAGGCAGGCA |
| | TCCTTGGAGATCAGTCCCATCACCTTCTTAACTGCCCAAACCCTTC |
| | TGATGGACCTTGGTCAATTTCTGCTGTCCTGCCATATTTCTTCCCAT |
| | CAGCATGATGGGATGGAGGCCTATGTAAAGGTGGATTCTTGCCCA |
| | GAGGAACCCCAGCTGAGGATGAAGAACAATGAGGAAGCAGAGGA |
| | TTATGATGATGATCTTACTGATAGTGAGATGGATGTGGTTAGGTTT |
| | GATGATGACAACAGCCCCAGCTTTATCCAGATTAGGTCTGTTGCTA |
| | AGAAGCATCCAAAGACATGGGTTCATTACATAGCTGCAGAAGAAG |
| | AGGACTGGGATTATGCCCCCCTAGTCCTTGCCCCAGATGATAGATC |
| | CTATAAATCTCAGTATCTGAACAATGGACCTCAGAGGATAGGCAG |
| | GAAGTATAAGAAAGTGAGATTCATGGCATATACAGATGAGACCTT |
| | CAAGACCAGAGAAGCAATCCAACATGAAAGTGGGATTCTGGGCCC |
| | ACTGCTGTATGGTGAGGTGGGTGACACCCTGCTGATAATCTTCAAG |
| | AATCAGGCCAGCAGGCCTTATAACATCTATCCACATGGGATAACA |
| | GATGTAAGGCCTCTGTATAGTAGAAGGCTGCCCAAAGGGGTGAAA |
| | CACCTGAAAGACTTTCCTATTCTTCCAGGTGAAATCTTCAAATACA |
| | AGTGGACAGTCACAGTGGAGGATGGGCCTACTAAAAGTGACCCCA |
| | GGTGCCTGACTAGGTACTATAGTTCCTTTGTTAACATGGAAAGGGA |
| | CCTAGCTAGTGGGCTTATAGGCCCCCTTCTGATCTGTTATAAAGAA |
| | TCTGTTGATCAGAGGGGCAACCAGATCATGTCAGACAAAAGGAAT |
| | GTGATACTTTTTAGTGTGTTTGATGAGAATAGGAGCTGGTACCTGA |
| | CTGAGAACATCCAGAGGTTTTTGCCCAACCCAGCTGGTGTGCAGCT |
| | TGAGGACCCAGAGTTTCAAGCCTCAAACATCATGCACAGTATCAA |
| | TGGCTATGTTTTTGATTCTCTTCAGCTGAGTGTTTGCCTGCATGAAG |
| | TAGCTTACTGGTACATCCTCTCAATTGGGGCTCAGACAGATTTCCT |
| | TTCAGTGTTCTTCAGTGGCTACACTTTCAAGCACAAAATGGTCTAT |
| | GAGGACACTCTGACCTTATTCCCTTTTAGTGGAGAGACAGTCTTTA |
| | TGAGTATGGAGAATCCTGGGCTGTGGATTCTTGGGTGCCACAACTC |
| | TGATTTCAGAAATAGGGGAATGACTGCCCTGCTAAAGGTGAGTAG |
| | TTGTGACAAAAACACAGGGGATTACTATGAGGACAGCTATGAGGA |
| | CATCAGTGCCTATCTGCTCTCAAAGAACAATGCAATAGAGCCTAG |
| | GTCCTTCTCACAGAATAGCAGGCATCCCTCAACCAGGCAAAAACA |
| | GTTTAATGCCACCACCATACCAGAGAATGATATTGAAAAGACTGA |
| | CCCTTGGTTTGCTCACAGGACCCCAATGCCCAAAATCCAAAATGTG |
| | TCTTCAAGTGACCTGTTAATGCTGTTGAGGCAGTCACCCACCCCAC |
| | ATGGGCTGTCCCTGTCAGATCTTCAAGAGGCCAAATATGAGACCTT |
| | CAGTGATGATCCTTCACCAGGAGCCATAGATTCTAACAACTCCCTG |
| | TCAGAAATGACACACTTCAGACCTCAGTTACATCATTCTGGGGATA |
| | TGGTGTTCACACCTGAATCTGGTCTCCAGCTGAGGTTAAATGAGAA |
| | ACTTGGGACTACAGCAGCTACTGAGCTGAAAAAACTTGATTTTAA |
| | GGTGAGCTCAACAAGCAATAACCTGATTTCCACCATTCCTAGTGAC |
| | AACCTAGCTGCTGGCACTGATAACACCTCTTCTCTGGGCCCTCCTT |
| | CAATGCCAGTACATTATGATTCCCAACTTGATACCACCCTCTTTGG |
| | GAAGAAGTCTAGCCCACTTACAGAATCAGGTGGCCCACTTAGTCT |
| | CTCAGAGGAGAATAATGATTCCAAACTTTTGGAGAGTGGGCTGAT |
| | GAACTCTCAAGAGAGCAGTTGGGGGAAGAATGTATCCCAGAGAG |
| | AGATAACCAGAACAACCCTTCAGTCTGACCAGGAGGAGATAGATT |
| | ATGATGACACTATCTCAGTTGAGATGAAAAAGGAGGACTTTGATA |
| | TCTATGATGAAGATGAAAATCAGTCTCCCAGGTCCTTCCAGAAAA |
| | AAACAAGGCATTACTTCATAGCAGCAGTAGAGAGACTGTGGGATT |
| | ATGGGATGTCATCCAGTCCTCATGTCCTGAGGAACAGAGCTCAGA |
| | GTGGCTCAGTGCCTCAATTCAAGAAAGTGGTGTTCCAGGAGTTCA |
| | CAGATGGCTCCTTCACACAGCCTCTCTACAGAGGAGAGCTCAATG |
| | AACACTTGGGGCTGCTGGGGCCCTATATCAGGGCAGAGGTGGAGG |
| | ATAATATTATGGTGACATTTAGGAACCAAGCCTCAAGGCCATACT |
| | CATTTTATTCTTCCCTGATCTCATATGAAGAGGATCAGAGGCAGGG |
| | GGCAGAACCTAGGAAGAATTTTGTAAAGCCTAATGAGACTAAGAC |
| | TTACTTTTGGAAGGTGCAACACCACATGGCTCCCACCAAAGATGA |
| | ATTTGATTGCAAGGCTTGGGCCTACTTCTCTGATGTGGATCTGGAG |
| | AAAGATGTGCATTCTGGCTTGATAGGTCCCCTACTGGTTTGTCATA |
| | CAAATACCCTGAACCCTGCACATGGCAGGCAGGTGACTGTGCAGG |
| | AGTTTGCTCTCTTCTTCACTATATTTGATGAGACCAAGTCCTGGTA |
| | CTTCACAGAGAATATGGAAAGAAACTGCAGGGCCCCATGTAATAT |
| | CCAGATGGAAGACCCCACCTTTAAGGAAAATTATAGGTTTCATGC |
| | TATTAATGGATATATAATGGACACCCTCCCAGGTCTGGTCATGGCA |
| | CAGGACCAGAGGATAAGGTGGTATCTGCTTTCTATGGGCTCTAAT |
| | GAAAATATTCATAGCATCCACTTTAGTGGGCATGTGTTCACTGTCA |
| | GGAAAAAAGAGGAGTATAAAATGGCCCTGTATAACCTGTACCCAG |
| | GAGTGTTTGAGACAGTAGAGATGCTTCCTAGTAAGGCTGGCATTT |
| | GGAGAGTAGAGTGCCTGATTGGAGAACATCTCCATGCTGGAATGA |
| | GCACCCTGTTCCTGGTCTATAGTAACAAGTGTCAGACCCCACTGGG |
| | CATGGCCTCAGGTCATATTAGGGACTTCCAAATCACTGCCAGTGGC |
| | CAATATGGGCAGTGGGCCCCCAAACTAGCCAGGTTGCACTATTCT |
| | GGGAGTATTAATGCCTGGTCTACAAAGGAACCATTTTCCTGGATCA |
| | AAGTGGATCTGCTGGCCCCAATGATCATACATGGAATTAAGACCC |
| | AGGGAGCCAGGCAGAAGTTTTCCTCCCTGTACATTAGTCAGTTCAT |
| | CATTATGTACAGTTTGGATGGGAAAAAGTGGCAGACATATAGGGG |
| | TAACAGTACAGGAACCCTTATGGTGTTCTTTGGTAATGTTGACAGC |
| | TCAGGAATCAAGCACAATATTTTCAACCCCCCAATCATAGCCAGG |
| | TACATCAGGCTCCACCCTACTCATTATAGCATCAGGAGCACCCTGA |
| | GAATGGAGCTTATGGGATGTGACCTAAACTCCTGTAGTATGCCTCT |
| | GGGAATGGAATCAAAGGCCATCTCTGATGCCCAAATTACAGCCTC |
| | ATCCTACTTTACAAACATGTTTGCTACTTGGTCACCATCCAAAGCT |
| | AGACTGCACCTGCAGGGGAGGTCAAATGCCTGGAGGCCACAAGTC |
| | AACAACCCCAAAGAATGGCTCCAAGTTGACTTTCAGAAGACTATG |
| | AAAGTGACTGGGGTGACTACTCAAGGGGTAAAGTCTCTTCTTACC |
| | AGTATGTATGTCAAAGAGTTTTTGATAAGCTCCTCCCAGGATGGCC |
| | ACCAGTGGACACTGTTTTTTCAGAATGGCAAGGTGAAAGTGTTCC |
| | AAGGAAACCAGGATTCTTTTACTCCAGTGGTGAACAGCCTGGACC |
| | CCCCCCTTCTGACCAGATACTTGAGAATCCACCCCCAGTCCTGGGT |
| | TCATCAGATTGCTCTGAGGATGGAAGTGCTGGGATGTGAGGCCCA |
| | GGACCTCTACTAA (SEQ ID NO. 180) |
| FVIII v10 | ATGCAGATTGAACTGTCTACCTGCTTCTTTCTGTGCCTCCTGAGGTT |
| (truncated | CTGTTTTAGTGCCACAAGGAGATATTACTTAGGGGCAGTTGAGCTG |
| CpG free, | TCCTGGGATTACATGCAGAGTGATTTAGGGGAGCTGCCAGTTGAT |
| GATC | GCTAGGTTCCCTCCAAGGGTGCCTAAGTCCTTCCCCTTTAATACCT |
| minimized) | CAGTGGTGTATAAGAAGACCCTTTTTGTAGAGTTCACTGACCACCT |
| | GTTCAATATAGCTAAACCCAGGCCACCTTGGATGGGGTTGTTAGG |
| | GCCAACAATTCAGGCAGAGGTTTATGACACAGTAGTGATAACCCT |
| | CAAGAATATGGCTAGTCACCCTGTGAGCCTGCATGCAGTAGGTGT |
| | GTCTTACTGGAAGGCTTCAGAGGGTGCTGAGTATGATGACCAGAC |
| | TTCTCAGAGAGAGAAGGAAGATGACAAAGTCTTTCCAGGGGGAAG |
| | TCATACATATGTTTGGCAGGTTCTTAAAGAGAATGGTCCTATGGCC |
| | AGTGACCCCCTGTGTCTGACTTACTCCTACCTGAGCCATGTGGACC |
| | TGGTGAAGGATTTAAACTCTGGGCTGATAGGTGCACTGCTGGTGT |
| | GTAGGGAAGGTAGCCTTGCCAAAGAGAAGACCCAAACACTCCACA |
| | AGTTCATATTGCTCTTTGCAGTTTTTGATGAAGGAAAAAGCTGGCA |
| | TAGTGAAACCAAAAACTCTTTAATGCAGGATAGGGATGCAGCTAG |
| | TGCTAGGGCCTGGCCAAAAATGCACACAGTCAATGGTTATGTGAA |
| | CAGAAGTCTTCCAGGTCTGATAGGCTGCCACAGGAAAAGTGTGTA |
| | CTGGCATGTGATAGGCATGGGAACCACCCCAGAAGTGCACTCAAT |
| | ATTCCTTGAGGGTCATACCTTCCTTGTGAGAAATCATAGGCAGGCA |
| | AGTTTGGAGATAAGTCCCATAACCTTCTTAACTGCCCAAACCCTTC |
| | TCATGGACTTAGGGCAATTTCTGCTGTCCTGCCATATCTCTTCCCA |
| | CCAGCATGATGGCATGGAGGCCTATGTAAAGGTGGATTCTTGCCC |
| | AGAGGAACCCCAGCTGAGAATGAAGAACAATGAGGAAGCAGAGG |
| | ATTATGATGATGACTTAACTGATAGTGAAATGGATGTGGTTAGGTT |
| | TGATGATGACAACAGCCCCAGCTTTATCCAGATTAGGTCTGTTGCT |
| | AAGAAGCACCCAAAGACATGGGTTCATTACATAGCTGCAGAAGAA |
| | GAGGACTGGGATTATGCCCCCCTTGTCCTTGCCCCAGATGATAGAA |
| | GTTACAAATCTCAGTATCTGAACAATGGACCTCAGAGGATAGGCA |
| | GGAAGTATAAGAAAGTGAGATTCATGGCATATACAGATGAGACCT |
| | TCAAGACCAGAGAAGCAATCCAACATGAAAGTGGGATTTTAGGGC |
| | CACTGCTGTATGGTGAGGTGGGTGACACCCTGCTGATAATCTTCAA |
| | GAATCAGGCCAGCAGGCCTTATAACATATATCCACATGGGATAAC |
| | AGATGTAAGGCCTCTGTATAGTAGAAGGCTGCCCAAAGGGGTGAA |
| | ACACCTGAAAGACTTTCCTATTCTTCCAGGTGAAATCTTCAAATAC |
| | AAGTGGACAGTCACAGTGGAGGATGGGCCTACTAAAAGTGACCCC |
| | AGGTGCCTGACTAGGTACTATAGTTCCTTTGTTAACATGGAAAGGG |
| | ACCTAGCTAGTGGGCTTATAGGCCCCCTTCTGATATGTTACAAAGA |
| | ATCTGTTGACCAGAGGGGCAACCAGATAATGTCAGACAAAAGGAA |
| | TGTGATACTTTTTAGTGTGTTTGATGAGAATAGGAGCTGGTACCTG |
| | ACTGAGAACATACAGAGGTTTTTGCCCAACCCAGCTGGTGTGCAG |
| | CTTGAGGACCCAGAGTTTCAAGCCTCAAACATAATGCACAGTATC |
| | AATGGCTATGTTTTTGATTCTCTTCAGCTGAGTGTTTGCCTGCATGA |
| | AGTAGCTTACTGGTACATACTCTCAATTGGGGCTCAGACAGATTTC |
| | CTTTCAGTGTTCTTCAGTGGCTACACTTTCAAGCACAAAATGGTCT |
| | ATGAGGACACTCTGACCTTATTCCCTTTTAGTGGAGAGACAGTCTT |
| | TATGAGTATGGAGAATCCTGGGCTGTGGATTTTAGGGTGCCACAA |
| | CTCTGATTTCAGAAATAGGGGAATGACTGCCCTGCTAAAGGTGAG |
| | TAGTTGTGACAAAAACACAGGGGATTACTATGAGGACAGCTATGA |
| | GGACATAAGTGCCTATCTGCTCTCAAAGAACAATGCAATAGAGCC |
| | TAGGTCCTTCTCACAGAATAGCAGGCACCCCTCAACCAGGCAAAA |
| | ACAGTTTAATGCCACCACCATACCAGAGAATGATATTGAAAAGAC |
| | TGACCCTTGGTTTGCTCACAGGACCCCAATGCCCAAAATCCAAAAT |
| | GTGTCTTCAAGTGACCTGTTAATGCTGTTGAGGCAGTCACCCACCC |
| | CACATGGGCTGTCCCTGTCAGATTTACAAGAGGCCAAGTATGAGA |
| | CCTTCAGTGATGACCCTTCACCAGGAGCCATAGATTCTAACAACTC |
| | CCTGTCAGAAATGACACACTTCAGACCTCAGTTACACCATTCTGGG |
| | GATATGGTGTTCACACCTGAATCTGGTCTCCAGCTGAGGTTAAATG |
| | AGAAATTAGGGACTACAGCAGCTACTGAGCTGAAGAAACTTGATT |
| | TTAAGGTGAGCTCAACAAGCAATAACCTGATTTCCACCATTCCTAG |
| | TGACAACCTAGCTGCTGGCACTGATAACACCTCTTCTTTAGGGCCT |
| | CCTTCAATGCCAGTACATTATGATTCCCAACTTGATACCACCCTCT |
| | TTGGGAAGAAGTCTAGCCCACTTACAGAATCAGGTGGCCCACTTA |
| | GTCTCTCAGAGGAGAATAATGATTCCAAACTTTTGGAGAGTGGGC |
| | TCATGAACTCTCAAGAGAGCAGTTGGGGGAAGAATGTATCCCAGA |
| | GAGAGATAACCAGAACAACCCTTCAGTCTGACCAGGAGGAGATAG |
| | ATTATGATGACACTATCTCAGTTGAAATGAAAAAGGAGGACTTTG |
| | ATATCTATGATGAGGATGAAAATCAGTCTCCCAGGTCCTTCCAGA |
| | AGAAAACAAGGCATTACTTCATAGCAGCAGTAGAGAGACTGTGGG |
| | ATTATGGCATGTCAAGTAGTCCTCATGTCCTGAGGAACAGAGCTC |
| | AGAGTGGCTCAGTGCCTCAATTCAAGAAAGTGGTGTTCCAGGAGT |
| | TCACAGATGGCTCCTTCACACAGCCTCTCTACAGAGGAGAGCTCA |
| | ATGAACACTTAGGGCTGTTAGGGCCCTATATTAGGGCAGAGGTGG |
| | AGGATAATATTATGGTGACATTTAGGAACCAAGCCTCAAGGCCAT |
| | ACTCATTCTATTCTTCCCTGATATCATATGAAGAGGACCAGAGGCA |
| | GGGGGCAGAACCTAGGAAGAATTTTGTAAAGCCTAATGAGACTAA |
| | GACTTACTTTTGGAAGGTGCAACACCACATGGCTCCCACCAAAGA |
| | TGAATTTGATTGCAAGGCTTGGGCCTACTTCTCTGATGTGGATCTG |
| | GAGAAAGATGTGCATTCTGGCTTGATAGGTCCCCTACTGGTTTGTC |
| | ATACAAATACCCTGAACCCTGCACATGGCAGGCAGGTGACTGTGC |
| | AGGAGTTTGCTCTCTTCTTCACTATCTTTGATGAGACCAAGTCCTG |
| | GTACTTCACAGAGAATATGGAAAGAAACTGCAGGGCCCCATGTAA |
| | TATCCAAATGGAAGACCCCACCTTTAAGGAAAATTATAGGTTTCAT |
| | GCTATTAATGGATATATAATGGACACCCTCCCAGGTCTGGTCATGG |
| | CACAGGACCAGAGGATAAGGTGGTATCTGCTTTCTATGGGCTCTA |
| | ATGAGAATATCCATAGCATACACTTTAGTGGGCATGTGTTCACTGT |
| | CAGGAAGAAAGAGGAGTACAAAATGGCCCTGTATAACCTGTACCC |
| | AGGAGTGTTTGAGACAGTAGAAATGCTTCCTAGTAAGGCTGGCAT |
| | TTGGAGAGTAGAGTGCCTGATTGGAGAACATTTACATGCTGGAAT |
| | GAGCACCCTGTTCCTGGTCTATAGTAACAAGTGTCAGACCCCATTA |
| | GGGATGGCCTCAGGTCATATTAGGGACTTCCAAATCACTGCCAGT |
| | GGCCAATATGGGCAGTGGGCCCCCAAACTAGCCAGGTTGCACTAT |
| | TCTGGGAGTATTAATGCCTGGTCTACAAAGGAACCATTTTCCTGGA |
| | TAAAAGTGGATTTACTGGCCCCAATGATAATACATGGAATTAAGA |
| | CCCAGGGAGCCAGGCAGAAGTTTTCCTCCCTGTACATTAGTCAGTT |
| | CATAATTATGTACAGTTTGGATGGGAAAAAGTGGCAGACATATAG |
| | GGGTAACAGTACAGGAACCCTTATGGTGTTCTTTGGTAATGTTGAC |
| | AGCTCAGGAATCAAGCACAATATCTTCAACCCCCCAATCATAGCC |
| | AGGTACATAAGGCTCCACCCTACTCATTATAGCATAAGGAGCACC |
| | CTGAGAATGGAGCTTATGGGCTGTGACCTAAACTCCTGTAGTATGC |
| | CTTTAGGGATGGAATCAAAGGCCATATCTGATGCCCAAATTACAG |
| | CCTCAAGTTACTTTACAAACATGTTTGCTACTTGGTCACCAAGTAA |
| | AGCTAGACTGCACCTGCAGGGGAGGTCAAATGCCTGGAGGCCACA |
| | AGTCAACAACCCCAAAGAATGGCTCCAAGTTGACTTTCAGAAGAC |
| | TATGAAAGTGACTGGGGTGACTACTCAAGGGGTAAAGTCTCTTCTT |
| | ACCAGTATGTATGTCAAAGAGTTTTTGATAAGCTCCTCCCAGGATG |
| | GCCACCAGTGGACACTGTTCTTTCAGAATGGCAAGGTGAAAGTGT |
| | TCCAAGGAAACCAGGATTCTTTTACTCCAGTGGTGAACAGCCTGG |
| | ACCCCCCTCTTCTGACCAGATACTTGAGAATCCACCCCCAGTCCTG |
| | GGTTCACCAGATTGCTCTGAGAATGGAAGTGTTAGGGTGTGAGGC |
| | CCAGGACCTCTACTAA (SEQ ID NO. 181) |
| Truncated | ATGCAAATAGAGCTCTCCACCTGCTTCTTTCTGTGCCTTTTGAGGTT |
| F8 (partial | CTGCTTTAGTGCCACCAGAAGATACTACCTGGGTGCAGTGGAACT |
| deletion of | GTCATGGGACTATATGCAAAGTGATCTAGGTGAGCTGCCTGTGGA |
| B domain | TGCAAGATTTCCTCCTAGAGTGCCAAAATCTTTTCCATTCAACACC |
| and linker | TCAGTAGTGTACAAAAAGACTTTATTTGTAGAATTTACTGATCACC |
| a3) | TTTTCAACATAGCTAAGCCAAGGCCACCCTGGATGGGTCTGCTAG |
| | GTCCTACCATCCAGGCTGAGGTTTATGATACAGTGGTCATTACACT |
| | TAAGAACATGGCTTCCCATCCTGTCAGTCTTCATGCTGTTGGTGTA |
| | TCCTACTGGAAAGCTAGTGAGGGAGCTGAATATGATGATCAGACC |
| | AGTCAAAGGGAGAAAGAAGATGATAAAGTCTTCCCTGGTGGAAGC |
| | CATACATATGTCTGGCAGGTCCTGAAAGAGAATGGTCCAATGGCC |
| | TCTGACCCACTGTGCCTTACCTACTCATATCTTTCTCATGTGGACCT |
| | GGTAAAAGACTTGAATAGTGGCCTCATTGGAGCCCTACTGGTATG |
| | TAGAGAAGGGAGTTTAGCCAAGGAAAAGACACAGACCTTGCACA |
| | AATTTATACTACTTTTTGCTGTATTTGATGAAGGGAAAAGTTGGCA |
| | CTCAGAAACAAAGAACTCCTTGATGCAGGATAGGGATGCTGCATC |
| | TGCTAGGGCCTGGCCTAAAATGCACACAGTCAATGGTTATGTAAA |
| | CAGGTCTCTGCCAGGTCTGATTGGATGCCACAGGAAATCAGTCTAT |
| | TGGCATGTGATTGGAATGGGCACCACTCCTGAAGTGCACTCAATA |
| | TTCCTAGAAGGTCACACATTTCTTGTGAGGAACCATAGGCAGGCCT |
| | CCTTGGAAATCTCCCCAATAACTTTCCTTACTGCTCAAACACTCTT |
| | GATGGACCTTGGACAGTTTCTACTGTCTTGTCATATCTCTTCCCACC |
| | AACATGATGGCATGGAAGCCTATGTCAAAGTAGACAGTTGTCCAG |
| | AGGAACCCCAACTAAGGATGAAAAATAATGAAGAAGCTGAAGAC |
| | TATGATGATGATCTTACTGATTCTGAAATGGATGTGGTCAGGTTTG |
| | ATGATGACAACTCTCCTTCCTTTATCCAAATTAGGTCAGTTGCCAA |
| | GAAGCATCCTAAAACTTGGGTACATTACATAGCTGCTGAAGAGGA |
| | GGACTGGGACTATGCTCCCTTAGTCCTAGCCCCAGATGACAGAAG |
| | TTATAAAAGTCAATATTTGAACAATGGCCCTCAGAGGATTGGTAG |
| | GAAGTACAAAAAAGTCAGGTTTATGGCATACACAGATGAAACCTT |
| | TAAGACTAGGGAAGCTATTCAGCATGAATCAGGAATCTTGGGACC |
| | TTTACTTTATGGGGAAGTTGGAGACACACTGTTGATTATATTTAAG |
| | AATCAAGCAAGCAGACCATATAACATCTACCCTCATGGAATCACT |
| | GATGTCAGGCCTTTGTATTCAAGGAGATTACCAAAAGGTGTAAAA |
| | CATTTGAAGGATTTTCCAATTCTGCCAGGAGAAATATTCAAATATA |
| | AATGGACAGTGACTGTAGAAGATGGGCCAACTAAATCAGATCCTA |
| | GGTGCCTGACCAGGTATTACTCTAGTTTTGTTAATATGGAGAGAGA |
| | TCTAGCTTCAGGACTAATTGGCCCTCTCCTCATCTGCTACAAAGAA |
| | TCTGTAGATCAAAGAGGAAACCAGATAATGTCAGACAAGAGGAAT |
| | GTCATCCTGTTTTCTGTATTTGATGAGAACAGGAGCTGGTACCTCA |
| | CAGAGAATATACAAAGGTTTCTCCCCAATCCAGCAGGAGTGCAGC |
| | TTGAGGACCCAGAGTTCCAAGCCTCCAACATCATGCACAGCATCA |
| | ATGGCTATGTTTTTGATAGTTTGCAGTTGTCAGTTTGTTTGCATGAG |
| | GTGGCATACTGGTACATTCTAAGCATTGGAGCACAGACTGACTTCC |
| | TTTCTGTCTTCTTCTCTGGATATACCTTCAAACACAAAATGGTCTAT |
| | GAAGACACACTCACCCTATTCCCATTCTCAGGAGAAACTGTCTTCA |
| | TGTCTATGGAAAACCCAGGTCTATGGATTCTGGGGTGCCACAACTC |
| | AGACTTTAGGAACAGAGGCATGACAGCCTTACTGAAGGTTTCTAG |
| | TTGTGACAAGAACACTGGTGATTATTATGAGGACAGTTATGAAGA |
| | TATTTCAGCATACTTGCTGAGTAAAAACAATGCCATTGAACCAAG |
| | AAGTTTCTCCCAGAATAGTAGACACCCTAGCACTAGGCAAAAGCA |
| | ATTTAATGCCACCACAATTCCAGAAAATGACATAGAGAAGACTGA |
| | CCCTTGGTTTGCACACAGAACACCTATGCCTAAAATACAAAATGTC |
| | TCCTCTAGTGATTTGTTGATGCTCTTGAGGCAGAGCCCTACTCCAC |
| | ATGGGCTATCCTTATCTGATCTCCAAGAAGCCAAATATGAGACTTT |
| | TTCTGATGATCCATCACCTGGAGCAATAGACAGTAATAACAGCCT |
| | GTCTGAAATGACACACTTCAGGCCACAGCTCCATCACAGTGGGGA |
| | CATGGTATTTACCCCTGAGAGTGGCCTCCAATTAAGATTAAATGAG |
| | AAACTGGGGACAACTGCAGCAACAGAGTTGAAGAAACTTGATTTC |
| | AAAGTTTCTAGTACATCAAATAATCTGATTTCAACAATTCCATCAG |
| | ACAATTTGGCAGCAGGTACTGATAATACAAGTTCCTTAGGACCCC |
| | CAAGTATGCCAGTTCATTATGATAGTCAATTAGATACCACTCTATT |
| | TGGCAAAAAGTCATCTCCCCTTACTGAGAGTGGTGGACCTCTGAG |
| | CTTGAGTGAAGAAAATAATGATTCAAAGTTGTTAGAATCAGGTTT |
| | AATGAATAGCCAAGAAAGTTCATGGGGAAAAAATGTATCTCAAGA |
| | GGAAATTGACTATGATGATACCATATCAGTTGAAATGAAGAAGGA |
| | AGATTTTGACATTTATGATGAGGATGAAAATCAGAGCCCCAGGAG |
| | CTTTCAAAAGAAAACAAGGCACTATTTTATTGCTGCAGTGGAGAG |
| | GCTCTGGGATTATGGGATGAGTAGCTCCCCACATGTTCTAAGAAA |
| | CAGGGCTCAGAGTGGCAGTGTCCCTCAGTTCAAGAAAGTTGTTTTC |
| | CAGGAATTTACTGATGGCTCCTTTACTCAGCCCTTATACAGGGGAG |
| | AACTAAATGAACATTTGGGACTACTGGGGCCATATATAAGAGCAG |
| | AAGTTGAAGATAATATCATGGTAACTTTCAGAAATCAGGCCTCTA |
| | GGCCCTATTCCTTCTATTCTAGCCTTATTTCTTATGAGGAAGATCA |
| | GAGGCAAGGAGCAGAACCTAGAAAAAACTTTGTCAAGCCTAATGA |
| | AACCAAAACTTACTTTTGGAAAGTGCAACATCATATGGCACCCAC |
| | TAAAGATGAGTTTGACTGCAAAGCCTGGGCTTATTTCTCTGATGTT |
| | GACCTGGAAAAAGATGTGCACTCAGGCCTGATTGGACCCCTTCTG |
| | GTCTGCCACACTAACACACTGAACCCTGCTCATGGGAGACAAGTG |
| | ACAGTACAGGAATTTGCTCTGTTTTTCACCATCTTTGATGAGACCA |
| | AAAGCTGGTACTTCACTGAAAATATGGAAAGAAACTGCAGGGCTC |
| | CCTGCAATATCCAGATGGAAGATCCCACTTTTAAAGAGAATTATA |
| | GGTTCCATGCAATCAATGGCTACATAATGGATACACTACCTGGCTT |
| | AGTAATGGCTCAGGATCAAAGGATTAGGTGGTATCTGCTCAGCAT |
| | GGGCAGTAATGAAAACATCCATTCTATTCATTTCAGTGGACATGTG |
| | TTCACTGTAAGGAAAAAAGAGGAGTATAAAATGGCACTGTACAAT |
| | CTCTATCCAGGTGTTTTTGAGACAGTGGAAATGTTACCATCCAAAG |
| | CTGGAATTTGGAGGGTGGAATGCCTTATTGGAGAGCATCTACATG |
| | CTGGGATGAGCACACTTTTTCTGGTGTACAGCAATAAGTGTCAGAC |
| | CCCCCTGGGAATGGCTTCTGGACACATTAGAGATTTTCAGATTACA |
| | GCTTCAGGACAATATGGACAGTGGGCCCCAAAGCTGGCCAGACTT |
| | CATTATTCAGGATCAATCAATGCCTGGAGCACCAAGGAGCCCTTTT |
| | CTTGGATCAAGGTGGATCTGTTGGCACCAATGATTATTCATGGCAT |
| | CAAGACCCAGGGTGCCAGGCAGAAGTTCTCCAGCCTCTACATCTC |
| | TCAGTTTATCATCATGTATAGTCTTGATGGGAAGAAGTGGCAGACT |
| | TATAGGGGAAATTCCACTGGAACCTTAATGGTCTTCTTTGGAAATG |
| | TGGATTCATCTGGGATAAAACACAATATTTTTAACCCTCCAATTAT |
| | TGCTAGGTACATCAGGTTGCACCCAACTCATTATAGCATTAGGAGC |
| | ACTCTTAGGATGGAGTTGATGGGCTGTGATTTAAATAGTTGCAGCA |
| | TGCCATTGGGAATGGAGAGTAAAGCAATATCAGATGCACAGATTA |
| | CTGCTTCATCCTACTTTACCAATATGTTTGCCACCTGGTCTCCTTCA |
| | AAAGCTAGGCTTCACCTCCAAGGGAGGAGTAATGCCTGGAGACCT |
| | CAGGTGAATAATCCAAAAGAGTGGCTGCAAGTGGACTTCCAGAAG |
| | ACAATGAAAGTCACAGGAGTAACTACTCAGGGAGTAAAATCTCTG |
| | CTTACCAGCATGTATGTGAAGGAGTTCCTCATCTCCAGCAGTCAAG |
| | ATGGCCATCAGTGGACTTTATTTTTTCAGAATGGCAAAGTAAAGGT |
| | TTTTCAGGGAAATCAAGACAGTTTCACACCTGTGGTGAACTCTCTG |
| | GACCCACCCTTACTGACTAGGTACCTTAGGATTCACCCCCAGAGTT |
| | GGGTGCACCAGATTGCCCTGAGGATGGAGGTTCTGGGCTGTGAGG |
| | CACAGGACCTCTACTGATGA (SEQ ID NO. 360) |

In one aspect, a codon optimized, engineered nucleic acid sequence encoding human FVIII is provided. In certain embodiments, an engineered human FVIII cDNA is provided herein (as SEQ ID NO: 175), which was designed to remove select nicking enzyme recognition sites compared to the native FVIII sequence (SEQ ID NO: 174). Preferably, the codon engineered FVIII coding sequence has less than about 80% identity or less to the full-length native FVIII coding sequence. In one embodiment, the codon optimized FVIII coding sequence has about 75% identity with the native FVIII coding sequence of SEQ ID NO: 174. In one embodiment, the engineered FVIII coding sequence is characterized by improved translation rate as compared to native FVIII following delivery. In one embodiment, the engineered FVIII coding sequence shares less than about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61% or less identity to the full length native FVIII coding sequence of SEQ ID NO: 174. In one embodiment, the codon optimized nucleic acid sequence is a variant of SEQ ID NO: 175. In another embodiment, the codon optimized nucleic acid sequence a sequence sharing about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61% or greater identity with SEQ ID NO: 175. In one embodiment, the codon optimized nucleic acid sequence is SEQ ID NO: 175. In another embodiment, the nucleic acid sequence is codon optimized for expression in humans. In other embodiments, a different FVIII coding sequence is selected.

A further example of a nucleic acid modification provided herein is CpG reduction. In certain embodiments, a CpG reduced nucleic acid encoding FVIII, such as human FVIII protein, or a functional fragment thereof, has 10 or fewer, 5 or fewer, or no more than 5 CpGs compared to wild-type sequence encoding human Factor FVIII.

A further example of a nucleic acid modification provided herein is reduction of restriction sites for select nicking endonucleases. In certain embodiments, a restriction site reduced nucleic acid encoding FVIII, such as human FVIII protein, or a functional fragment thereof, has 5 or fewer restriction sites for nicking endonucleases compared to wild-type sequence encoding human Factor FVIII. In certain embodiments, a restriction site reduced nucleic acid encoding FVIII, or a functional fragment thereof, has no restriction sites for nicking endonucleases compared to the wild-type sequence encoding human Factor FVIII. In some embodiments, the expression cassette comprises nucleic acid modifications to reduce CpG and restriction sites for selected nicking endonucleases.

In one aspect, a CpG minimized, engineered nucleic acid sequence encoding human FVIII translating to a F328S amino acid substitution is provided. In certain embodiments, an engineered human FVIII cDNA translating to a F328S mutation is provided herein (as SEQ ID NO: 176), which was also designed to remove select nicking enzyme recognition sites and minimize CpG motifs as compared to the native FVIII sequence. Preferably, the CpG minimized FVIII coding sequence has less than about 90% identity, preferably about 85% identity or less to the full-length native FVIII coding sequence. In one embodiment, the CpG minimized FVIII coding sequence has about 81% identity with the native FVIII coding sequence of SEQ ID NO: 174. In one embodiment, the CpG minimized FVIII coding sequence is characterized by a reduced activation for host immune reaction as compared to native FVIII sequence following delivery into host cells. In one embodiment, the CpG minimized FVIII coding sequence shares less than about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61% or less identity to the full length native FVIII coding sequence of SEQ ID NO: 174. In one embodiment, the CpG minimized nucleic acid sequence is a variant of SEQ ID NO: 176. In another embodiment, the CpG minimized nucleic acid sequence has a sequence sharing about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61% or greater identity with SEQ ID NO: 176. In one embodiment, the CpG minimized nucleic acid sequence is SEQ ID NO: 176.

In some embodiments, a hairpin-ended DNA molecule, as described herein, encodes a fusion protein comprising a full length, fragment or portion of a FVIII protein fused to another sequence (e.g., an N or C terminal fusion). In some embodiments, the N or C terminal sequence is a signal sequence or a cellular targeting sequence. In further embodiments, hairpin-ended DNA molecule, as described herein, encodes a FVIII protein having a full or partial B domain deletion, which may involve the remaining portions of the B domain linked to the A2 and A3 domains, as in the wildtype amino acid sequence or it may include one or more spacer amino acids to replace part or all of the deleted domains. In further embodiments, hairpin-ended DNA molecule, as described herein, encodes a FVIII protein comprising a full or partial B domain deletion as well as partial linker a3 deletion, which may involve the remaining portions of the B domain linked to the A2 domain and partial linker a3, as in the wildtype amino acid sequence or it may include one or more spacer amino acids to replace part or all of the deletions in the domains or linker.

In certain embodiments, the truncated linker a3 has a deletion of the N-terminal portion of the linker a3 domain, wherein the number of amino acids deleted from the N-terminal portion of the linker a3 domain ranges from about 1 to about 10, about 1 to about 20, about 1 to about 30, or about 1 to about 40 amino acids. In specific embodiments, the truncated linker a3 has a deletion of nine N-terminal amino acids of the linker a3. In one embodiment, the truncated FVIII variant comprising a truncated N-terminal of the linker a3 is characterized by a higher procoagulant activity as compared to native FVIII following expression by host cells.

In some embodiments the truncated B domain is the N-terminal portion of the B domain, wherein the number of amino acids in the truncated N-terminal portion of the B domain ranges from about 20 to about 300, about 25 to about 300, about 29 to about 300, about 29 to about 269, about 29 to about 250, about 30 to about 300, about 30 to about 250, about 50 to about 300, about 50 to about 250, about 100 to about 300, about 100 to about 500, about 100 to about 250, about 150 to about 300, about 150 to about 250, about 200 to about 300, or about 250 to about 300 amino acids. In some embodiments the hairpin-ended DNA molecule can comprise truncated versions of a wildtype Factor VIII B-domain or peptides engineered to replace the B-domain of a Factor VIII polypeptide. As used herein, a Factor VIII linker is positioned between the C-terminus of a Factor VIII heavy chain and the N-terminus of a Factor VIII light chain in a Factor VIII variant polypeptide in accordance with some embodiments. Non-limiting examples of B-domain substituted linkers are disclosed in U.S. Patent Application Publication Nos. 2013/024960, 2015/0071883, and 2015/0158930; and PCT Publication Nos. WO 2014/064277 and WO 2014/127215, the disclosures of which are hereby incorporated by reference, in their entireties, for all purposes.

In a specific embodiment, a hairpin-ended DNA molecule comprising a nucleic acid sequence encoding a truncated human FVIII protein comprising a partial B-domain and truncated linker a3 (B-NA3) is provided, wherein the truncated protein encoded by the DNA molecule is characterized by a deletion of amino acids 985-1677 compared to the native FVIII sequence. In one embodiment, the truncated B-NA3 FVIII protein coding sequence shares less than about 99%, less than about 98%, less than about 97%, less than about 96%, less than about 95%, less than about 94%, less than about 93%, less than about 92%, less than about 91%, less than about 90%, less than about 89%, less than about 88%, less than about 87%, less than about 86%, less than about 85%, less than about 84%, less than about 83%, less than about 82%, less than about 81%, less than about 80%, less than about 79%, less than about 78%, less than about 77%, less than about 76%, less than about 75%, less than about 74%, less than about 73%, less than about 72%, less than about 71%, less than about 70%, less than about 69%, less than about 68%, less than about 67%, less than about 66%, less than about 65%, less than about 64%, less than about 63%, less than about 62%, or less than about 61% identity to the full length native FVIII coding sequence of SEQ ID NO: 174. In one embodiment, the B-NA3 truncated FVIII protein sequence is a variant of SEQ ID NO: 358. In another embodiment, the B-NA3 truncated FVIII protein sequence has a sequence sharing about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, or greater than 61%, identity with SEQ ID NO: 358. In one embodiment, B-NA3 truncated FVIII protein sequence is SEQ ID NO: 358.

In a specific embodiment, the hairpin-ended DNA molecule comprises a nucleic acid sequence encoding a truncated human FVIII protein, wherein the truncated protein encoded by the DNA molecule has the amino acid sequence of SEQ ID NO: 354. In one embodiment, the truncated FVIII protein sequence is a variant of SEQ ID NO: 354. In another embodiment, the truncated FVIII encoding protein sequence has a sequence sharing about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, or greater than 61%, identity with SEQ ID NO: 354. In one embodiment, truncated FVIII protein sequence is SEQ ID NO: 354.

In a specific embodiment, the hairpin-ended DNA molecule comprises a nucleic acid sequence encoding a truncated human FVIII protein, wherein the truncated protein encoded by the DNA molecule has the amino acid sequence of SEQ ID NO: 355. In one embodiment, the truncated FVIII protein sequence is a variant of SEQ ID NO: 355. In another embodiment, the truncated FVIII protein sequence has a sequence sharing about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, or greater than 61%, identity with SEQ ID NO: 355. In one embodiment, truncated FVIII protein sequence is SEQ ID NO: 355.

In a specific embodiment, the hairpin-ended DNA molecule comprises a nucleic acid sequence encoding a truncated human FVIII protein, wherein the truncated protein encoded by the DNA molecule has the amino acid sequence of SEQ ID NO: 356. In one embodiment, the truncated FVIII protein sequence is a variant of SEQ ID NO: 356. In another embodiment, the truncated FVIII protein sequence has a sequence sharing about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, or greater than 61%, identity with SEQ ID NO: 356. In one embodiment, truncated FVIII protein sequence is SEQ ID NO: 356.

In a specific embodiment, the hairpin-ended DNA molecule comprises a nucleic acid sequence encoding a truncated human FVIII protein, wherein the truncated protein encoded by the DNA molecule has the amino acid sequence of SEQ ID NO: 357. In one embodiment, the truncated FVIII protein sequence is a variant of SEQ ID NO: 357. In another embodiment, the truncated FVIII protein sequence has a sequence sharing about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, or greater than 61%, identity with SEQ ID NO: 357. In one embodiment, truncated FVIII protein sequence is SEQ ID NO: 357.

In a specific embodiment, the hairpin-ended DNA molecule comprises a nucleic acid sequence encoding a truncated human FVIII protein, wherein the truncated protein encoded by the DNA molecule has the amino acid sequence of SEQ ID NO: 360. In one embodiment, the truncated FVIII protein sequence is a variant of SEQ ID NO: 360. In another embodiment, the truncated FVIII protein sequence has a sequence sharing about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, or greater than 61%, identity with SEQ ID NO: 360. In one embodiment, truncated FVIII protein sequence is SEQ ID NO: 360.

In a specific embodiment, the hairpin-ended DNA molecule comprises a nucleic acid sequence encoding a truncated human FVIII protein, wherein the truncated protein encoded by the DNA molecule has the amino acid sequence of SEQ ID NO: 378. In one embodiment, the truncated FVIII protein sequence is a variant of SEQ ID NO: 378. In another embodiment, the truncated FVIII protein sequence has a sequence sharing about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, or greater than 61%, identity with SEQ ID NO: 378. In one embodiment, truncated FVIII protein sequence is SEQ ID NO: 378.

In a specific embodiment, the hairpin-ended DNA molecule comprises a nucleic acid sequence encoding a truncated human FVIII protein, wherein the truncated protein encoded by the DNA molecule has the amino acid sequence of SEQ ID NO: 382. In one embodiment, the truncated FVIII protein sequence is a variant of SEQ ID NO: 382. In another embodiment, the truncated FVIII protein sequence has a sequence sharing about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, or greater than 61%, identity with SEQ ID NO: 382. In one embodiment, truncated FVIII protein sequence is SEQ ID NO: 382.

In a specific embodiment, the hairpin-ended DNA molecule comprises a nucleic acid sequence encoding a truncated human FVIII protein, wherein the truncated protein encoded by the DNA molecule has the amino acid sequence of SEQ ID NO: 386. In one embodiment, the truncated FVIII protein sequence is a variant of SEQ ID NO: 386. In another embodiment, the truncated FVIII protein sequence has a sequence sharing about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, or greater than 61%, identity with SEQ ID NO: 386. In one embodiment, truncated FVIII protein sequence is SEQ ID NO: 386.

In a specific embodiment, the hairpin-ended DNA molecule comprises a nucleic acid sequence encoding a truncated human FVIII protein, wherein the truncated protein encoded by the DNA molecule has the amino acid sequence of SEQ ID NO: 390. In one embodiment, the truncated FVIII protein sequence is a variant of SEQ ID NO: 390. In another embodiment, the truncated FVIII protein sequence has a sequence sharing about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, or greater than 61%, identity with SEQ ID NO: 390. In one embodiment, truncated FVIII protein sequence is SEQ ID NO: 390.

In a specific embodiment, the hairpin-ended DNA molecule comprises a nucleic acid sequence encoding a truncated human FVIII protein, wherein the truncated protein encoded by the DNA molecule has the amino acid sequence of SEQ ID NO: 394. In one embodiment, the truncated FVIII protein sequence is a variant of SEQ ID NO: 394. In another embodiment, the truncated FVIII protein sequence has a sequence sharing about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, or greater than 61%, identity with SEQ ID NO: 394. In one embodiment, truncated FVIII protein sequence is SEQ ID NO: 394.

In a specific embodiment, the hairpin-ended DNA molecule comprises a nucleic acid sequence encoding a truncated human FVIII protein, wherein the truncated protein encoded by the DNA molecule has the amino acid sequence of SEQ ID NO: 398. In one embodiment, the truncated FVIII protein sequence is a variant of SEQ ID NO: 398. In another embodiment, the truncated FVIII protein sequence has a sequence sharing about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, or greater than 61%, identity with SEQ ID NO: 398. In one embodiment, truncated FVIII protein sequence is SEQ ID NO: 398.

In a specific embodiment, the hairpin-ended DNA molecule comprises a nucleic acid sequence encoding a truncated human FVIII protein, wherein the truncated protein encoded by the DNA molecule has the amino acid sequence of SEQ ID NO: 402. In one embodiment, the truncated FVIII protein sequence is a variant of SEQ ID NO: 402. In another embodiment, the truncated FVIII protein sequence has a sequence sharing about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, or greater than 61%, identity with SEQ ID NO: 402. In one embodiment, truncated FVIII protein sequence is SEQ ID NO: 402.

In a specific embodiment, the hairpin-ended DNA molecule comprises a nucleic acid sequence encoding a truncated human FVIII protein, wherein the truncated protein encoded by the DNA molecule has the amino acid sequence of SEQ ID NO: 406. In one embodiment, the truncated FVIII protein sequence is a variant of SEQ ID NO: 406. In another embodiment, the truncated FVIII protein sequence has a sequence sharing about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, or greater than 61%, identity with SEQ ID NO: 406. In one embodiment, truncated FVIII protein sequence is SEQ ID NO: 406.

In a specific embodiment, an expression cassette comprises a FVIII transgene that is at least 60%, at least 70%, at least 80% or at least 90% identical to the sequence set forth in SEQ ID NO: 174. In a specific embodiment, an expression cassette comprises a FVIII transgene that is at least 60%, at least 70%, at least 80% or at least 90% identical to the sequence set forth in SEQ ID NO: 175. In a specific embodiment, an expression cassette comprises a FVIII transgene that is at least 60%, at least 70%, at least 80% or at least 90% identical to the sequence set forth in SEQ ID NO: 176. In a specific embodiment, an expression cassette comprises a FVIII transgene that is at least 60%, at least 70%, at least 80% or at least 90% identical to the sequence set forth in SEQ ID NO: 177. In a specific embodiment, an expression cassette comprises a FVIII transgene that is at least 60%, at least 70%, at least 80% or at least 90% identical to the sequence set forth in SEQ ID NO: 178. In a specific embodiment, an expression cassette comprises a FVIII transgene that is at least 60%, at least 70%, at least 80% or at least 90% identical to the sequence set forth in SEQ ID NO: 179. In a specific embodiment, an expression cassette comprises a FVIII transgene that is at least 60%, at least 70%, at least 80% or at least 90% identical to the sequence set forth in SEQ ID NO: 180. In a specific embodiment, an expression cassette comprises a FVIII transgene that is at least 60%, at least 70%, at least 80% or at least 90% identical to the sequence set forth in SEQ ID NO: 379. In a specific embodiment, an expression cassette comprises a FVIII transgene that is at least 60%, at least 70%, at least 80% or at least 90% identical to the sequence set forth in SEQ ID NO: 380. In a specific embodiment, an expression cassette comprises a FVIII transgene that is at least 60%, at least 70%, at least 80% or at least 90% identical to the sequence set forth in SEQ ID NO: 381. In a specific embodiment, an expression cassette comprises a FVIII transgene that is at least 60%, at least 70%, at least 80% or at least 90% identical to the sequence set forth in SEQ ID NO: 383. In a specific embodiment, an expression cassette comprises a FVIII transgene that is at least 60%, at least 70%, at least 80% or at least 90% identical to the sequence set forth in SEQ ID NO: 384. In a specific embodiment, an expression cassette comprises a FVIII transgene that is at least 60%, at least 70%, at least 80% or at least 90% identical to the sequence set forth in SEQ ID NO: 385. In a specific embodiment, an expression cassette comprises a FVIII transgene that is at least 60%, at least 70%, at least 80% or at least 90% identical to the sequence set forth in SEQ ID NO: 387. In a specific embodiment, an expression cassette comprises a FVIII transgene that is at least 60%, at least 70%, at least 80% or at least 90% identical to the sequence set forth in SEQ ID NO: 388. In a specific embodiment, an expression cassette comprises a FVIII transgene that is at least 60%, at least 70%, at least 80% or at least 90% identical to the sequence set forth in SEQ ID NO: 389. In a specific embodiment, an expression cassette comprises a FVIII transgene that is at least 60%, at least 70%, at least 80% or at least 90% identical to the sequence set forth in SEQ ID NO: 391. In a specific embodiment, an expression cassette comprises a FVIII transgene that is at least 60%, at least 70%, at least 80% or at least 90% identical to the sequence set forth in SEQ ID NO: 392. In a specific embodiment, an expression cassette comprises a FVIII transgene that is at least 60%, at least 70%, at least 80% or at least 90% identical to the sequence set forth in SEQ ID NO: 393. In a specific embodiment, an expression cassette comprises a FVIII transgene that is at least 60%, at least 70%, at least 80% or at least 90% identical to the sequence set forth in SEQ ID NO: 395. In a specific embodiment, an expression cassette comprises a FVIII transgene that is at least 60%, at least 70%, at least 80% or at least 90% identical to the sequence set forth in SEQ ID NO: 396. In a specific embodiment, an expression cassette comprises a FVIII transgene that is at least 60%, at least 70%, at least 80% or at least 90% identical to the sequence set forth in SEQ ID NO: 397. In a specific embodiment, an expression cassette comprises a FVIII transgene that is at least 60%, at least 70%, at least 80% or at least 90% identical to the sequence set forth in SEQ ID NO: 399. In a specific embodiment, an expression cassette comprises a FVIII transgene that is at least 60%, at least 70%, at least 80% or at least 90% identical to the sequence set forth in SEQ ID NO: 400. In a specific embodiment, an expression cassette comprises a FVIII transgene that is at least 60%, at least 70%, at least 80% or at least 90% identical to the sequence set forth in SEQ ID NO: 401. In a specific embodiment, an expression cassette comprises a FVIII transgene that is at least 60%, at least 70%, at least 80% or at least 90% identical to the sequence set forth in SEQ ID NO: 403. In a specific embodiment, an expression cassette comprises a FVIII transgene that is at least 60%, at least 70%, at least 80% or at least 90% identical to the sequence set forth in SEQ ID NO: 404. In a specific embodiment, an expression cassette comprises a FVIII transgene that is at least 60%, at least 70%, at least 80% or at least 90% identical to the sequence set forth in SEQ ID NO: 405. In a specific embodiment, an expression cassette comprises a FVIII transgene that is at least 60%, at least 70%, at least 80% or at least 90% identical to the sequence set forth in SEQ ID NO: 407. In a specific embodiment, an expression cassette comprises a FVIII transgene that is at least 60%, at least 70%, at least 80% or at least 90% identical to the sequence set forth in SEQ ID NO: 408. In a specific embodiment, an expression cassette comprises a FVIII transgene that is at least 60%, at least 70%, at least 80% or at least 90% identical to the sequence set forth in SEQ ID NO: 409.

In a specific embodiment, an expression cassette comprises a FVIII transgene that is identical to the sequence set forth in SEQ ID NO: 174. In a specific embodiment, an expression cassette comprises a FVIII transgene that is identical to the sequence set forth in SEQ ID NO: 175. In a specific embodiment, an expression cassette comprises a FVIII transgene that is identical to the sequence set forth in SEQ ID NO: 176. In a specific embodiment, an expression cassette comprises a FVIII transgene that is identical to the sequence set forth in SEQ ID NO: 177. In a specific embodiment, an expression cassette comprises a FVIII transgene that is identical to the sequence set forth in SEQ ID NO: 178. In a specific embodiment, an expression cassette comprises a FVIII transgene that is identical to the sequence set forth in SEQ ID NO: 179. In a specific embodiment, an expression cassette comprises a FVIII transgene that is identical to the sequence set forth in SEQ ID NO: 180. In a specific embodiment, an expression cassette comprises a FVIII transgene that is identical to the sequence set forth in SEQ ID NO: 379. In a specific embodiment, an expression cassette comprises a FVIII transgene that is identical to the sequence set forth in SEQ ID NO: 380. In a specific embodiment, an expression cassette comprises a FVIII transgene that is identical to the sequence set forth in SEQ ID NO: 381. In a specific embodiment, an expression cassette comprises a FVIII transgene that is identical to the sequence set forth in SEQ ID NO: 383. In a specific embodiment, an expression cassette comprises a FVIII transgene that is identical to the sequence set forth in SEQ ID NO: 384. In a specific embodiment, an expression cassette comprises a FVIII transgene that is identical to the sequence set forth in SEQ ID NO: 385. In a specific embodiment, an expression cassette comprises a FVIII transgene that is identical to the sequence set forth in SEQ ID NO: 387. In a specific embodiment, an expression cassette comprises a FVIII transgene that is identical to the sequence set forth in SEQ ID NO: 388. In a specific embodiment, an expression cassette comprises a FVIII transgene that is identical to the sequence set forth in SEQ ID NO: 389. In a specific embodiment, an expression cassette comprises a FVIII transgene that is identical to the sequence set forth in SEQ ID NO: 391. In a specific embodiment, an expression cassette comprises a FVIII transgene that is identical to the sequence set forth in SEQ ID NO: 392. In a specific embodiment, an expression cassette comprises a FVIII transgene that is identical to the sequence set forth in SEQ ID NO: 393. In a specific embodiment, an expression cassette comprises a FVIII transgene that is identical to the sequence set forth in SEQ ID NO: 395. In a specific embodiment, an expression cassette comprises a FVIII transgene that is identical to the sequence set forth in SEQ ID NO: 396. In a specific embodiment, an expression cassette comprises a FVIII transgene that is identical to the sequence set forth in SEQ ID NO: 397. In a specific embodiment, an expression cassette comprises a FVIII transgene that is identical to the sequence set forth in SEQ ID NO: 399. In a specific embodiment, an expression cassette comprises a FVIII transgene that is identical to the sequence set forth in SEQ ID NO: 400. In a specific embodiment, an expression cassette comprises a FVIII transgene that is identical to the sequence set forth in SEQ ID NO: 401. In a specific embodiment, an expression cassette comprises a FVIII transgene that is identical to the sequence set forth in SEQ ID NO: 403. In a specific embodiment, an expression cassette comprises a FVIII transgene that is identical to the sequence set forth in SEQ ID NO: 404. In a specific embodiment, an expression cassette comprises a FVIII transgene that is identical to the sequence set forth in SEQ ID NO: 405. In a specific embodiment, an expression cassette comprises a FVIII transgene that is identical to the sequence set forth in SEQ ID NO: 407. In a specific embodiment, an expression cassette comprises a FVIII transgene that is identical to the sequence set forth in SEQ ID NO: 408. In a specific embodiment, an expression cassette comprises a FVIII transgene that is identical to the sequence set forth in SEQ ID NO: 409.

The term “percent (%) identity”, “sequence identity”, “percent sequence identity”, or “percent identical” in the context of FVIII encoding nucleic acid sequences refers to the residues in the two sequences which are the same when aligned for correspondence. The length of sequence identity comparison may be over the full-length of the genome, the full-length of a gene coding sequence, or a fragment of at least about 500 to 5000 nucleotides, is desired. However, identity among smaller fragments, e.g. of at least about nine nucleotides, usually at least about 20 to 24 nucleotides, at least about 28 to 32 nucleotides, at least about 36 or more nucleotides, may also be desired.

Percent identity may be readily determined for amino acid sequences over the full-length of a protein, polypeptide, about 32 amino acids, about 330 amino acids, or a peptide fragment thereof or the corresponding nucleic acid sequence coding sequences. A suitable amino acid fragment may be at least about 8 amino acids in length, and may be up to about 700 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Identity may be determined by preparing an alignment of the sequences and through the use of a variety of algorithms and/or computer programs known in the art or commercially available [e.g., BLAST, ExPASy; ClustalO; FASTA; using, e.g., Needleman-Wunsch algorithm, Smith-Waterman algorithm]. Alignments are performed using any of a variety of publicly or commercially available Multiple Sequence Alignment Programs. Sequence alignment programs are available for amino acid sequences, e.g., the “Clustal Omega”, and “Clustal X”, programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27 (13): 2682-2690 (1999). Multiple sequence alignment programs are also available for nucleic acid sequences. Examples of such programs include, “Clustal Omega”, “Clustal W”, “CAP Sequence Assembly”, “BLAST”, “MAP”, and “MEME”, which are accessible through Web Servers on the internet.

Codon-optimized coding regions can be designed by various different methods. This optimization may be performed using methods which are available on-line (e.g., GeneArt), published methods, or a company which provides codon optimizing services, e.g., DNA2.0 (Menlo Park, CA). Suitably, the entire length of the open reading frame (ORF) for the product is modified. However, in some embodiments, only a fragment of the ORF may be altered. By using one of these methods, one can apply the frequencies to any given polypeptide sequence, and produce a nucleic acid fragment of a codon-optimized coding region which encodes the polypeptide. A number of options are available for performing the actual changes to the codons or for synthesizing the codon-optimized coding regions designed as described herein. Such modifications or synthesis can be performed using standard and routine molecular biological manipulations well known to those of ordinary skill in the art.

The FVIII expression cassette may be located at any suitable distance of base pairs from either the 5′ and/or 3′ ITR closing pair (as described in section 5.4.1) to allow or to maintain efficient transcription of said expression cassette in host cells. In some embodiments the distance between the expression cassette and the 5′ ITR and the distance between the expression cassette and the 3′ ITR closing pair are identical. In some embodiments the distance between the expression cassette and the 5′ ITR and the distance between the expression cassette and the 3′ ITR closing pair are not identical. In some embodiments the distance between the expression cassette and/or the 3′ ITR closing pair and the distance between the expression cassette the 5′ ITR closing pair is least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, at least 315, at least 320, at least 325, at least 330, at least 335, at least 340, at least 345, at least 350, at least 355, at least 360, at least 365, at least 370, at least 375, at least 380, at least 385, at least 390, at least 395, or at least 400 nucleotides. In some embodiments the distance between the expression cassette and the 3′ ITR closing pair and/or the distance between the expression cassette and the 5′ ITR closing pair is about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, or about 400 nucleotides.

By “engineered nucleic acid sequence” is meant that the nucleic acid sequences encoding the FVIII protein described herein are assembled and placed into any suitable genetic element, e.g., naked DNA, phage, transposon, cosmid, episome, etc., which transfers the FVIII sequences carried thereon to a host cell, e.g., for generating non-viral delivery systems (e.g., RNA-based systems, naked DNA, or the like) or for generating viral vectors in a packaging host cell and/or for delivery to a host cells in a subject. In one embodiment, the genetic element is a circular plasmid. The methods used to make such engineered constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

In one embodiment, the nucleic acid sequence encoding FVIII further comprises a nucleic acid encoding a tag polypeptide covalently linked thereto. The tag polypeptide may be selected from known “epitope tags” including, without limitation, a myc tag polypeptide, a glutathione-S-transferase tag polypeptide, luciferase protein tag polypeptide, a green fluorescent protein tag polypeptide, a myc-pyruvate kinase tag polypeptide, a His6 tag polypeptide, an influenza virus hemagglutinin tag polypeptide, a flag tag polypeptide, and a maltose binding protein tag polypeptide. In some aspects, hairpin ended vectors expressing an FVIII protein linked to a reporter polypeptide may be used for diagnostic purposes, as well as to determine efficacy or as markers of the hairpin ended vectors' activity in the subject to which they are administered.

In yet another embodiment, the expression cassette comprises regulatory elements known and used in the art to regulate (promote, inhibit and/or turn on/off the expression of the ORF). Such regulatory elements include, for example, 5′-untranslated region (UTR), 3′-UTR, or both the 5′UTR and the 3′UTR. In some further embodiments, the expression cassette comprises any one or more features provided in this Section 5.4.3 in any combination or permutation.

Untranslated regions are nucleic acid sections of a polynucleotide before a start codon (5′ UTR) and after a stop codon (3′ UTR) that are not translated. In some embodiments, a polynucleotide of the invention comprising an open reading frame (ORF) encoding a Factor VIII polypeptide further comprises UTR (e.g., a 5′UTR or functional fragment thereof, a 3′UTR or functional fragment thereof, or a combination thereof). Additional exemplary translational regulatory activities provided by components, structures, elements, motifs, and/or specific sequences comprising polynucleotides include, but are not limited to, mRNA stabilization or destabilization (Baker & Parker (2004) Curr Opin Cell Biol 16 (3): 293-299), translational activation (Villalba et al., (2011) Curr Opin Genet Dev 21 (4): 452-457), and translational repression (Blumer et al., (2002) Mech Dev 110 (1-2): 97-112). Studies have shown that naturally occurring, cis-acting RNA elements can confer their respective functions when used to modify, by incorporation into, heterologous polynucleotides (Goldberg-Cohen et al., (2002) J Biol Chem 277 (16): 13635-13640). A UTR can be homologous or heterologous to the coding region in a polynucleotide. In some embodiments, the UTR is homologous to the ORF encoding the Factor VIII polypeptide. In some embodiments, the UTR is heterologous to the ORF encoding the Factor VIII polypeptide. In some embodiments, the polynucleotide comprises two or more 5′UTRs or functional fragments thereof, each of which have the same or different nucleotide sequences. In some embodiments, the polynucleotide comprises two or more 3′UTRs or functional fragments thereof, each of which have the same or different nucleotide sequences. In some embodiments, the 5′UTR and the 3′UTR can be heterologous. In some embodiments, the 5′UTR can be derived from a different species than the 3′UTR. In some embodiments, the 3′UTR can be derived from a different species than the 5′UTR. UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

The 5′ UTRs or 3′ UTRs of the hairpin ended DNA molecules of the present disclosure can in some embodiments contain sequences that destabilize the resulting mRNA transcript by cellular process. In certain embodiments, the mRNA transcript of the hairpin ended DNA molecule can be destabilized by cleavage of the mRNA (e.g. by a riboendonuclease) as a response to increased endoplasmic reticulum (ER) stress. In specific embodiments, the ER stress activates the riboendonuclease which in turn cleaves mRNA transcripts at specific sites or motifs. The ectopic expression of polypeptides (e.g. FVIII) can induce ER stress, hence it may be advantageous to engineer in a sequence comprises an ER stress dependent riboendonuclease recognition motif. In some embodiments the recognition motif folds into a loop and a stem. In specific embodiments, the stem comprises 4 nucleotides that form an energetically stable stem as well as a loop consisting of an approximately 7 nt the consensus transcribed RNA sequence of CNGCAGN (whereby N can be a A, G, T or C, SEQ ID NO: 333).

In certain embodiments, a mouse FVIII, hemoglobin alpha locus (e.g. Hemoglobin Subunit Alpha 1), Hemoglobin Subunit Alpha 2, or Hemoglobin beta locus (e.g. Hemoglobin Subunit Beta) 5′UTR and/or 3′UTR sequence can be used with the methods and compositions of the invention. In certain embodiments, a rabbit FVIII, hemoglobin alpha locus (e.g. Hemoglobin Subunit Alpha 1), Hemoglobin Subunit Alpha 2, or Hemoglobin beta locus (e.g. Hemoglobin Subunit Beta) 5′UTR and/or 3′UTR sequence can be used with the methods and compositions of the invention. In certain embodiments, a human FVIII, hemoglobin alpha locus (e.g. Hemoglobin Subunit Alpha 1), Hemoglobin Subunit Alpha 2, or Hemoglobin beta locus (e.g. Hemoglobin Subunit Beta) 5′UTR and/or 3′UTR sequence can be used with the methods and compositions of the invention. In certain embodiments, the 5′UTR and/or 3′UTR sequence of the present disclosure comprises a nucleotide sequence at least about 60%, at least about 70%, at least about 80%, at least about 90%, at least about 95%, at least about 96%, at least about 97%, at least about 98%, at least about 99%, or about 100% identical to SEQ ID NO: 333 flanked by a 4 nt stem. In certain embodiments, the expression cassette open reading frame can comprise nucleotide sequence at least about 60%, at least about 70%, at least about 80%, at least about 90%, at least about 95%, at least about 96%, at least about 97%, at least about 98%, at least about 99%, or about 100% identical to SEQ ID NO: 333 flanked by a 4 nt stem.

The expression cassette can comprise a protein coding sequence in its ORF (sense strand). Alternatively, the expression cassette can comprise the complementary sequence of the protein coding ORF (anti-sense strand) and the regulatory components and/or other signals for the cellular machinery to produce a sense strand DNA/RNA and the corresponding protein. In some embodiments, the expression cassette comprises a FVIII protein sequence without intron. In other embodiments, the expression cassette comprises a FVIII protein sequence with intron, which is removed upon transcription and splicing. The expression cassette can also comprise various numbers of ORFs or transcription units. In one embodiment, the expression cassette comprises 1, 2, 3, 4, 5, 6, 7, 8, 9, 10, 11, 12, 13, 14, 15, 16, 17, 18, 19, or 20 ORFs. In another embodiment, the expression cassette comprises 1, 2, 3, 4, 5, 6, 7, 8, 9, 10, 11, 12, 13, 14, 15, 16, 17, 18, 19, or 20 transcription units.

The expression cassettes can also comprise one or more transcriptional regulatory element, one or more posttranscriptional regulatory elements, or both one or more transcriptional regulatory element and one or more posttranscriptional regulatory elements. Such regulatory elements are any sequences that allow, contribute, or modulate the functional regulation of the nucleic acid molecule, including replication, duplication, transcription, splicing, translation, stability and/or transport of the nucleic acid or one of its derivative (e.g. mRNA) into the host cell or organism. Such regulatory elements include, but are not limited to, a promoter, an enhancer, a polyadenylation signal, translation stop codon, a ribosome binding element, a transcription terminator, selection markers, origin of replication, etc.

In some embodiments, the expression cassette comprises an enhancer. Any enhancer sequence known to those skilled in the art in view of the present disclosure can be used. In some embodiments, the enhancer is a liver-specific enhancer. In some embodiments, the enhancer can be an enhancer sequence of the human actin gene, human myosin gene, human hemoglobin gene, human muscle creatine gene, transthyretin gene, alpha-1-antitrypsin gene, albumin gene, apolipoprotein E gene, FVIII gene or a viral enhancer, such as one from CMV, HA, RSV, or EBV. In certain specific embodiments, the enhancer can be Woodchuck HBV Post-transcriptional regulatory element (WPRE), intron/exon sequence derived from human apolipoprotein A1 precursor (ApoAI), untranslated R-U5 domain of the human T-cell leukemia virus type 1 (HTLV-1) long terminal repeat (LTR), a splicing enhancer, a synthetic rabbit β-globin intron, a P5 promoter of an AAV, or any combination thereof. In certain embodiments, the enhancer comprises a nucleotide sequence at least about 60%, at least about 70%, at least about 80%, at least about 90%, at least about 95%, at least about 96%, at least about 97%, at least about 98%, at least about 99%, or about 100% identical to SEQ ID NO: 416. In certain embodiments, the enhancer comprises the nucleotide sequence of SEQ ID NO: 416.

As described above, the expression cassette can comprise a promoter to control expression of a protein of interest. Promoters include any nucleotide sequence that initiates the transcription of an operably linked nucleotide sequence. Promoters can be a constitutive, inducible, or repressible. A promoter can be derived from sources including viral, bacterial, fungal, plants, insects, and animals. A promoter can be a homologous promoter (e.g., derived from the same genetic source) or a heterologous promoter (e.g., derived from a different genetic source). In some embodiments, a promoters can be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter (CMV-IE), Epstein Barr virus (EBV) promoter, or a Rous sarcoma virus (RSV) promoter. In other embodiments, a promoter can be a promoter from a human gene such as human actin, human myosin, human hemoglobin, human muscle creatine, or human metallothionein. In further embodiments, a promoter can also be a tissue specific promoter, such as a muscle or skin specific promoter, natural or synthetic to promote expression in cells or tissues in which expression of FVIII is desirable such as in cells or tissues in which FVIII expression is desirable in FVIII-deficient patients. In certain embodiments, the promoter is a liver-specific promoter. In certain embodiments, the promoter comprises a nucleotide sequence at least about 60%, at least about 70%, at least about 80%, at least about 90%, at least about 95%, at least about 96%, at least about 97%, at least about 98%, at least about 99%, or about 100% identical to SEQ ID NO: 340, 341, 342, 347, 410, 411, 412, 413, 414, or 415. In certain embodiments, the promoter comprises the nucleotide sequence of SEQ ID NO: 340. In certain embodiments, the promoter comprises the nucleotide sequence of SEQ ID NO: 341. In certain embodiments, the promoter comprises the nucleotide sequence of SEQ ID NO: 342. In certain embodiments, the promoter comprises the nucleotide sequence of SEQ ID NO: 347. In certain embodiments, the promoter comprises the nucleotide sequence of SEQ ID NO: 410. In certain embodiments, the promoter comprises the nucleotide sequence of SEQ ID NO: 411. In certain embodiments, the promoter comprises the nucleotide sequence of SEQ ID NO: 412. In certain embodiments, the promoter comprises the nucleotide sequence of SEQ ID NO: 413. In certain embodiments, the promoter comprises the nucleotide sequence of SEQ ID NO: 414. In certain embodiments, the promoter comprises the nucleotide sequence of SEQ ID NO: 415. Exemplary regulatory elements can be found in Table 19.

In a particular embodiment, the promoter is a muscle-specific promoter. Non-limiting examples of muscle-specific promoters include the muscle creatine kinase (MCK) promoter. Non-limiting examples of suitable muscle creatine kinase promoters are human muscle creatine kinase promoters and truncated murine muscle creatine kinase [(tMCK) promoters] (Wang B et al, Construction and analysis of compact muscle-selective promoters for AAV vectors. Gene Ther. 2008 November; 15 (22): 1489-99) (representative GenBank Accession No. AF188002). Human muscle creatine kinase has the Gene ID No. 1158 (representative GenBank Accession No. NC 000019.9). Other examples of muscle-specific promoters include a synthetic promoter C5.12 (spC5.12, alternatively referred to herein as “C5.12”), such as the spC5.12 or the spC5.12 promoter (disclosed in Wang et al., Gene Therapy volume 15, pages 1489-1499 (2008)), the MHCK7 promoter (Salva et al. Mol Ther. 2007 February; 15 (2): 320-9), myosin light chain (MLC) promoters, for example MLC2 (Gene 1D No. 4633; representative GenBank Accession No. NG 007554.1); myosin heavy chain (MHC) promoters, for example alpha-MHC (Gene 1D No. 4624; representative GenBank Accession No. NG 023444.1); desmin promoters (Gene ID No. 1674; representative GenBank Accession No. NG 008043.1); cardiac troponin C promoters (Gene ID No. 7134; representative GenBank Accession No. NG 008963.1); troponin I promoters (Gene ID Nos. 7135, 7136, and 7137; representative GenBank Accession Nos. NG 016649.1, NG 011621.1, and NG_007866.2,); myoD gene family promoters (Weintraub et al., Science, 251, 761 (1991); Gene ID No. 4654; representative GenBank Accession No. NM 002478); alpha actin promoters (Gene ID Nos. 58, 59, and 70; representative GenBank Accession Nos. NG 006672.1, NG 011541.1, and NG 007553.1,); beta actin promoters (Gene ID No. 60; representative GenBank Accession No. NG 007992.1); gamma actin promoters (Gene ID No. 71 and 72; representative GenBank Accession No. NG 011433.1 and NM 001199893); muscle-specific promoters residing within intron 1 of the ocular form of Pitx3 (Gene ID No. 5309) (Coulon et al; the muscle-selective promoter corresponds to residues 11219-11527 of representative GenBank Accession No. NG 008147); and the promoters described in US Patent Publication US 2003/0157064, and CK6 promoters (Wang et al 2008 doi: 10.1038/gt.2008.104). In another particular embodiment, the muscle-specific promoter is the E-Syn promoter described in Wang et al., Gene Therapy volume 15, pages 1489-1499 (2008), comprising the combination of a MCK-derived enhancer and of the spC5.12 promoter. In a particular embodiment of the disclosure, the muscle-specific promoter is selected in the group consisting of a spC5.12 promoter, the MHCK7 promoter, the E-syn promoter, a muscle creatine kinase myosin light chain (MLC) promoter, a myosin heavy chain (MHC) promoter, a cardiac troponin C promoter, a troponin I promoter, a myoD gene family promoter, an alpha actin promoter, an beta actin promoter, an gamma actin promoter, a muscle-specific promoter residing within intron 1 of the ocular form of Pitx3, a CK6 promoter, a CK8 promoter and an Actal promoter. In a particular embodiment, the muscle-specific promoter is selected in the group consisting of the spC5.12, desmin and MCK promoters. In a further embodiment, the muscle-specific promoter is selected in the group consisting of the spC5.12 and MCK promoters. In a particular embodiment, the muscle-specific promoter is the spC5.12 promoter.

In a particular embodiment, the promoter is a liver-specific promoter. Non-limiting examples of liver-specific promoters include the alpha-1 antitrypsin promoter (hAAT), the transthyretin promoter, the albumin promoter, the thyroxine-binding globulin (TBG) promoter, the LSP promoter (comprising a thyroid hormone-binding globulin promoter sequence, two copies of an alpha-microglobulin/bikunin enhancer sequence, and a leader sequence—Ill, C. R., et al. (1997). Optimization of the human FVIII complementary DNA expression plasmid for gene therapy of hemophilia A. Blood Coag. Fibrinol. 8: S23-S30), etc. Other useful liver-specific promoters are known in the art, for example those listed in the Liver Specific Gene Promoter Database compiled the Cold Spring Harbor Laboratory (http://rulai.cshl.edu/LSPD/). A preferred liver-specific promoter in the context of the disclosure is the hAAT promoter.

In another particular embodiment, the promoter is a neuron-specific promoter. Non-limiting examples of neuron-specific promoters include, but are not limited to the following: synapsin-1 (Syn) promoter, neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al. Neuron, 15:373-84 (1995)), among others which will be apparent to the skilled artisan. In a particular embodiment, the neuron-specific promoter is the Syn promoter. Other neuron-specific promoters include, without limitation: synapsin-2 promoter, tyrosine hydroxylase promoter, dopamine b-hydroxylase promoter, hypoxanthine phosphoribosyltransferase promoter, low affinity NGF receptor promoter, and choline acetyl transferase promoter (Bejanin et al., 1992; Carroll et al., 1995; Chin and Greengard, 1994; Foss-Petter et al., 1990; Harrington et al., 1987; Mercer et al., 1991; Patei et al., 1986). Representative promoters specific for the motor neurons include, without limitation, the promoter of the Calcitonin Gene-Related Peptide (CGRP), a known motor neuron-derived factor. Other promoters functional in motor neurons include the promoters of Choline Acetyl Transferase (ChAT), Neuron Specific Enolase (NSE), Synapsin and Hb9. Other neuron-specific promoters useful in the present disclosure include, without limitation: GFAP (for astrocytes), Calbindin 2 (for interneurons), Mnxl (motorneurons), Nestin (neurons), Parvalbumin, Somatostatin and Plpl (oligodendrocytes and Schwann cells). In another particular embodiment, the promoter is a ubiquitous promoter. Representative ubiquitous promoters include the cytomegalovirus enhancer/chicken beta actin (CAG) promoter, the cytomegalovirus enhancer/promoter (CMV) (optionally with the CMV enhancer) [see, e.g., Boshart et al, Cell, 41:521-530 (1985)], the PGK promoter, the SV40 early promoter, the retroviral Rous sarcoma virus (RSV) LTR promoter (optionally with the RSV enhancer), the dihydrofolate reductase promoter, the b-actin promoter, the phosphoglycerol kinase (PGK) promoter, and the EF1 alpha promoter. In addition, the promoter may also be an endogenous promoter such as the albumin promoter or the F8 promoter. In a particular embodiment, the promoter is associated to an enhancer sequence, such as a cis-regulatory module (CRMs) or an artificial enhancer sequence. CRMs useful in the practice of the present disclosure include those described in Rincon et al., Mol Ther. 2015 January; 23 (1): 43-52, Chuah et al., Mol Ther. 2014 September; 22 (9): 1605-13 or Nair et al., Blood. 2014 May 15; 123 (20): 3195-9.

Other regulatory elements that are, in particular, able to enhance muscle-specific expression of genes, in particular expression in cardiac muscle and/or skeletal muscle, are those disclosed in WO2015110449. Particular examples of nucleic acid regulatory elements that comprise an artificial sequence include the regulatory elements that are obtained by rearranging the transcription factor binding sites (TFBS) that are present in the sequences disclosed in WO2015110449. Said rearrangement may encompass changing the order of the TFBSs and/or changing the position of one or more TFBSs relative to the other TFBSs and/or changing the copy number of one or more of the TFBSs. For example, a nucleic acid regulatory element for enhancing muscle-specific gene expression, in particular cardiac and skeletal muscle-specific gene expression, may comprise binding sites for E2A, HNH 1, NF1, C/EBP, LRF, MyoD, and SREBP; or for E2A, NF1, p53, C/EBP, LRF, and SREBP; or for E2A, HNH 1, HNF3a, HNF3b, NF1, C/EBP, LRF, MyOD, and SREBP; or E2A, HNF3a, NF1, C/EBP, LRF, MyoD, and SREBP; or for E2A, HNF3a, NF1, CEBP, LRF, MyOD, and SREBP; or for HNF4, NF1, RSRFC4, C/EBP, LRF, and MyoD, or NF1, PPAR, p53, C/EBP, LRF, and MyoD. For example, a nucleic acid regulatory element for enhancing muscle-specific gene expression, in particular skeletal muscle-specific gene expression, may also comprise binding sites for E2A, NF1, SRFC, p53, C/EBP, LRF, and MyoD; or for E2A, NF1, C/EBP, LRF, MyoD, and SREBP; or for E2A, HNF3a, C/EBP, LRF, MyOD, SEREBP, and Tall b; or for E2A, SRF, p53, C/EBP, LRF, MyoD, and SREBP; or for HNF4, NF1, RSRFC4, C/EBP, LRF, and SREBP; or for E2A, HNF3a, HNF3b, NF1, SRF, C/EBP, LRF, MyoD, and SREBP; or for E2A, CEBP, and MyoD. In further examples, these nucleic acid regulatory elements comprise at least two, such as 2, 3, 4, or more copies of one or more of the TFBSs recited before. Other regulatory elements that are, in particular, able to enhance liver-specific expression of genes, are those disclosed in WO2009130208.

TABLE 19
Exemplary Regulatory Elements

| Descrip- | |
|---|---|
| tion | Sequence |
| Endo- | taacacattcttgtttgtgcagaactgag |
| genous | ctcaatgataacatttttatagcaaccct |
| F8 | gatcaagtttcttctcataatctcttaca |
| Pro- | ctttgaggcccctgcaggggccctcactc |
| moter | tccctaataaacattaacctgagtagggt |
| | gtttgagctcaccatggctacattctgat |
| | gtaaagagatatatcctatacctgggcca |
| | aatgtaaacagcctggaaaagtgttaggt |
| | taaaaacaaaacaaaataaataaatgaat |
| | aaatgccaggtggttatgagtgctattga |
| | gaaaaatgaagccaagagggatatcagtg |
| | atgcaggtgggggtaaagagcttacaaca |
| | taaatgtggtgttccatatttaaacctca |
| | ttcaacagggaagattggagctgaaatgt |
| | gaaggagttgtgggagtggaactacgtgg |
| | aaatctgggggaaaggtgttttgggtaaa |
| | agaaatagcaagtgttgaggtccaggggc |
| | atgagtgtgcttgatattttagggaagag |
| | taaggagaccagtataaccagagtgagat |
| | gagactacagaggtcaggagaaagggcat |
| | gcagaccatgtgggatgctctaggaccta |
| | ggccatggtaaagatgtagggttttaccc |
| | tgatggaggtcagaagccattggaggatt |
| | ctgagaagaggagtgacaggactcgcttt |
| | atagttttaaattataactataaattata |
| | gtttttaaaacaatagttgcctaacctca |
| | tgttatatgtaaaactacagttttaaaaa |
| | ctataaattcctcatactggcagcagtgt |
| | gaggggcaagggcaaaagcagagagacta |
| | acaggttgctggttactcttgctagtgca |
| | agtgaattctagaatcttcgacaacatcc |
| | agaacttctcttgctgctgccactcagga |
| | agagggttggagtaggctaggaataggag |
| | cacaaattaaagctcctgttcactttgac |
| | ttctccatccctctcctcctttccttaaa |
| | ggttctgattaaagcagacttatgcccct |
| | actgctctcagaagtgaatgggttaagtt |
| | tagcagcctcccttttgctacttcagttc |
| | ttcctgtggctgcttcccactgataaaaa |
| | ggaagcaatcctatcggttactgcttagt |
| | gctgagcacatccagtgggtaaagttcct |
| | taaaatgctctgcaaagaaattgggactt |
| | ttcattaaatcagaaattttacttttttc |
| | ccctcctgggagctaaagatattttagag |
| | aagaattaaccttttgcttctccagttga |
| | acatttgtagcaataagtc |
| | (SEQ ID NO: 340) |
| Endo- | tgggatgctctaggacctaggccatggta |
| genous | aagatgtagggttttaccctgatggaggt |
| F8 | cagaagccattggaggattctgagaagag |
| pro- | gagtgacaggactcgctttatagttttaa |
| moter | attataactataaattatagtttttaaaa |
| 2 | caatagttgcctaacctcatgttatatgt |
| | aaaactacagttttaaaaactataaattc |
| | ctcatactggcagcagtgtgaggggcaag |
| | ggcaaaagcagagagactaacaggttgct |
| | ggttactcttgctagtgcaagtgaattct |
| | agaatcttcgacaacatccagaacttctc |
| | ttgctgctgccactcaggaagagggttgg |
| | agtaggctaggaataggagcacaaattaa |
| | agctcctgttcactttgacttctccatcc |
| | ctctcctcctttccttaaaggttctgatt |
| | aaagcagacttatgcccctactgctctca |
| | gaagtgaatgggttaagtttagcagcctc |
| | ccttttgctacttcagttcttcctgtggc |
| | tgcttcccactgataaaaaggaagcaatc |
| | ctatcggttactgcttagtgctgagcaca |
| | tccagtgggtaaagttccttaaaatgctc |
| | tgcaaagaaattgggacttttcattaaat |
| | cagaaattttacttttttcccctcctggg |
| | agctaaagatattttagagaagaattaac |
| | cttttgcttctccagttgaacatttgtag |
| | caataagtc |
| | (SEQ ID NO: 341) |
| Endo- | gaacttctcttgctgctgccactcaggaa |
| genous | gagggttggagtaggctaggaataggagc |
| F8 | acaaattaaagctcctgttcactttgact |
| pro- | tctccatccctctcctcctttccttaaag |
| moter | gttctgattaaagcagacttatgccccta |
| 3 | ctgctctcagaagtgaatgggttaagttt |
| | agcagcctcccttttgctacttcagttct |
| | tcctgtggctgcttcccactgataaaaag |
| | gaagcaatcctatcggttactgcttagtg |
| | ctgagcacatccagtgggtaaagttcctt |
| | aaaatgctctgcaaagaaattgggacttt |
| | tcattaaatcagaaattttacttttttcc |
| | cctcctgggagctaaagatattttagaga |
| | agaattaaccttttgcttctccagttgaa |
| | catttgtagcaataagtc |
| | (SEQ ID NO: 342) |
| ApoE | AGGCTCAGAGGCACACAGGAGTTTCTGGG |
| en- | CTCACCCTGCCCCCTTCCAACCCCTCAGT |
| hancer- | TCCCATCCTCCAGCAGCTGTTTGTGTGCT |
| TTR | GCCTCTGAAGTCCACACTGAACAAACTTC |
| pro- | AGCCTACTCATGTCCCTAAAATGGGCAAA |
| moter- | CTTTGCAAGCAGCAAACAGCAAACACACA |
| | GCCCTCCCTGCCTGCTGACCTTGGAGCTG |
| | GGGCAGAGGTCAGAGACCTCTCTGGGCCC |
| | ATGCCACCTCCAACATCCACTCGACCCCT |
| | TGGAATTTCGGTGGAGAGGAGCAGAGGTT |
| | GTCCTGGCGTGGTTTAGGTAGTGTGAGAG |
| | GGGTCTGTCTGCACATTTCGTAGAGCGAG |
| | TGTTCCGATACTCTAATCTCCCTAGGCAA |
| | GGTTCATATTTGTGTAGGTTACTTATTCT |
| | CCTTTTGTTGACTAAGTCAATAATCAGAA |
| | TCAGCAGGTTTGGAGTCAGCTTGGCAGGG |
| | ATCAGCAGCCTGGGTTGGAAGGAGGGGGT |
| | ATAAAAGCCCCTTCACCAGGAGAAGCCGT |
| | CACACAGATCCACAAGCTCCTG |
| | (SEQ ID NO: 347) |

In some embodiments, the expression cassette comprises an intron. In some embodiments, an intron is located in the ORF, in the 5′ UTR, and/or in the 3′ UTR. In certain embodiments, the intron is derived from a human, mouse, rabbit, or viral genome. In certain embodiments, the intron derived from a source genome (e.g. human, mouse, rabbit, or viral genome) can be used in its entirety, or a truncated or chimeric version of the intron may be used. In certain embodiments, the intron is derived from the minute virus of mice. In other embodiments, the intron is derived from a FVIII gene, a Transthyretin gene, an Alpha-1-Antitrypsin gene, an Albumin gene, an Apolipoprotein gene, the Hemoglobin beta locus (e.g. the Hemoglobin Subunit Beta), the Immunoglobulin Heavy Locus (e.g. IGHV).

In some embodiments, the expression cassette can comprise a polyadenylation, termination signal, or both a polyadenylation and termination signal. Any polyadenylation signal known to those skilled in the art in view of the present disclosure can be used. In some embodiments, the polyadenylation signal can be a SV40 polyadenylation signal, AAV2 polyadenylation signal (bp 4411-4466, NC_001401), a polyadenylation signal from the Herpes Simplex Virus Thymidine Kinase Gene, LTR polyadenylation signal, bovine growth hormone (bGH) polyadenylation signal, human growth hormone (hGH) polyadenylation signal, or human B-globin polyadenylation signal. In some embodiments, the polyadenylation signal is a synthetic polyA such as a synthetic polyA described in Levitt et al, Genes Dev. 1989 July; 3 (7): 1019-25, doi: 10.1101/gad.3.7.1019.

In some embodiments the expression cassette can have various sizes to accommodate one or more ORFs of various lengths. In certain embodiments, the size of expression cassette is at least 0.2 kb, at least 0.3 kb, at least 0.4 kb, at least 0.5 kb, at least 0.6, at least kb, at least 0.7 kb, at least 0.8 kb, at least 0.9 kb, at least 1 kb, at least 1.5 kb, at least 2 kb, at least 2.5 kb, at least 3 kb, at least 3.5 kb, at least 4 kb, at least 4.5 kb, at least 5 kb, at least 5.5 kb, at least 6 kb, at least 6.5 kb, at least 7 kb, at least 7.5 kb, at least 8 kb, at least 8.5 kb, at least 9 kb, at least 9.5 kb, at least 10 kb, at least 15 kb, at least 20 kb, at least 25 kb, at least 30 kb, at least 35 kb, at least 40 kb, at least 45 kb, at least 50 kb, at least 55 kb, at least 60 kb, at least 65 kb, at least 70 kb, at least 75 kb, or at least 80 kb. In one specific embodiment, the expression cassette is at least 4.5 kb. In another specific embodiment, the expression cassette is at least 4.6 kb. In yet another specific embodiment, the expression cassette is at least 4.7 kb. In a further specific embodiment, the expression cassette is at least 4.8 kb. In one specific embodiment, the expression cassette is at least 4.9 kb. In another specific embodiment, the expression cassette is at least 5 kb. In other embodiments, the size of the expression cassette is about 0.2 kb, about 0.3 kb, about 0.4 kb, about 0.5 kb, about 0.6, about kb, about 0.7 kb, about 0.8 kb, about 0.9 kb, about 1 kb, about 1.5 kb, about 2 kb, about 2.5 kb, about 3 kb, about 3.5 kb, about 4 kb, about 4.5 kb, about 5 kb, about 5.5 kb, about 6 kb, about 6.5 kb, about 7 kb, about 7.5 kb, about 8 kb, about 8.5 kb, about 9 kb, about 9.5 kb, about 10 kb, about 15 kb, about 20 kb, about 25 kb, about 30 kb, about 35 kb, about 40 kb, about 45 kb, about 50 kb, about 55 kb, about 60 kb, about 65 kb, about 70 kb, about 75 kb, or about 80 kb. In one specific embodiment, the expression cassette is about 8 kb. In another specific embodiment, the expression cassette is about 7.6 kb. In yet another specific embodiment, the expression cassette is about 7.7 kb. In a further specific embodiment, the expression cassette is about 7.8 kb. In one specific embodiment, the expression cassette is about 7.9 kb. In another specific embodiment, the expression cassette is about 5 kb. The expression cassette can also comprise various numbers of genes of interest (“transgenes”). In one embodiment, the expression cassette comprises 1, 2, 3, 4, 5, 6, 7, 8, 9, 10, 11, 12, 13, 14, 15, 16, 17, 18, 19, or 20 transgenes. In some specific embodiment, the expression cassette comprises one transgene. In some embodiments, the transgenes are recombinant genes. In some further embodiments, the transgenes comprise cDNA sequences (e.g. no introns in the transgenes).

In some embodiment, the DNA molecules provided herein do not have the size limitations of encapsidated AAV vectors, thus enabling delivery of a large-size expression cassette to provide efficient transgene. In certain embodiments, the DNA molecules provided herein comprise an expression cassette equal to or larger than the size of any natural AAV genome.

The expression cassette can have various positions relative to the inverted repeat. In some embodiments, the expression cassette is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or at least 100 nucleotides apart from the inverted repeat. In certain embodiments, the expression cassette is at least 0.2 kb, at least 0.3 kb, at least 0.4 kb, at least 0.5 kb, at least 0.6, at least 0.7 kb, at least 0.8 kb, at least 0.9 kb, at least 1 kb, at least 1.5 kb, or at least 2 kb apart from the inverted repeat. In other embodiments, the expression cassette is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, or about 100 nucleotides apart from the inverted repeat. In further embodiments, the expression cassette is about 0.2 kb, about 0.3 kb, about 0.4 kb, about 0.5 kb, about 0.6, about 0.7 kb, about 0.8 kb, about 0.9 kb, about 1 kb, about 1.5 kb, or about 2 kb apart from the inverted repeat. In one embodiment, the inverted repeat in this paragraph is the first inverted repeat as described in Sections 3 and 5.4 (including 5.4.1). In another embodiment, the inverted repeat in this paragraph is the second inverted repeat as described in 5.4 (including 5.4.1) In yet another embodiment, the inverted repeat in this paragraph is both the first and the second inverted repeat as described in 5.4 (including 5.4.1).

The expression cassettes can also comprise one or more transcriptional regulatory elements, one or more posttranscriptional regulatory elements, or both one or more transcriptional regulatory element and one or more posttranscriptional regulatory elements. Such regulatory elements are any sequences that allow, contribute, or modulate the functional regulation of the nucleic acid molecule, including replication, duplication, transcription, splicing, translation, stability and/or transport of the nucleic acid or one of its derivative (e.g. mRNA) into the host cell or organism. Such regulatory elements include, but are not limited to, a promoter, an enhancer, a polyadenylation signal, translation stop codon, a ribosome binding element, a transcription terminator, selection markers, and/or origin of replication.

The expression cassette can have various sizes to accommodate one or more ORFs of various lengths. In certain embodiments, the size of expression cassette is at least at least 2 kb, at least 2.5 kb, at least 3 kb, at least 3.5 kb, at least 4 kb, at least 4.5 kb, at least 5 kb, at least 5.5 kb, at least 6 kb, at least 6.5 kb, at least 7 kb, at least 7.5 kb, at least 8 kb, at least 8.5 kb, at least 9 kb, at least 9.5 kb, at least 10 kb, at least 15 kb, at least 20 kb, at least 25 kb, at least 30 kb, at least 35 kb, at least 40 kb, at least 45 kb, at least 50 kb, at least 55 kb, at least 60 kb, at least 65 kb, at least 70 kb, at least 75 kb, or at least 80 kb. In one specific embodiment, the expression cassette is at least 7.5 kb. In another specific embodiment, the expression cassette is at least 7.6 kb. In yet another specific embodiment, the expression cassette is at least 7.7 kb. In a further specific embodiment, the expression cassette is at least 7.8 kb. In one specific embodiment, the expression cassette is at least 7.9 kb. In another specific embodiment, the expression cassette is at least 8 kb. In other embodiments, the size of the expression cassette is about 2 kb, about 2.5 kb, about 3 kb, about 3.5 kb, about 4 kb, about 4.5 kb, about 5 kb, about 5.5 kb, about 6 kb, about 6.5 kb, about 7 kb, about 7.5 kb, about 8 kb, about 8.5 kb, about 9 kb, about 9.5 kb, about 10 kb, about 15 kb, about 20 kb, about 25 kb, about 30 kb, about 35 kb, about 40 kb, about 45 kb, about 50 kb, about 55 kb, about 60 kb, about 65 kb, about 70 kb, about 75 kb, or about 80 kb. In one specific embodiment, the expression cassette is about 7.5 kb. In another specific embodiment, the expression cassette is about 7.6 kb. In yet another specific embodiment, the expression cassette is about 7.7 kb. In a further specific embodiment, the expression cassette is about 7.8 kb. In one specific embodiment, the expression cassette is about 7.9 kb. In another specific embodiment, the expression cassette is about 8 kb. The expression cassette can also comprise various numbers of genes of interest (“transgenes”). In one embodiment, the expression cassette comprises 1, 2, 3, 4, 5, 6, 7, 8, 9, 10, 11, 12, 13, 14, 15, 16, 17, 18, 19, or 20 transgenes. In some specific embodiment, the expression cassette comprises one transgene. In some embodiments, the transgenes are recombinant genes. In some further embodiments, the transgenes comprise cDNA sequences (e.g. no introns in the transgenes).

Additionally, the expression cassette can comprise at least 4000 nucleotides, at least 5000 nucleotides, at least 10,000 nucleotides, at least 20,000 nucleotides, at least 30,000 nucleotides, at least 40,000 nucleotides, or at least 50,000 nucleotides. In some embodiments, the expression cassette can comprise any range of from about 4000 to about 10,000 nucleotides, from about 10,000 to about 50,000 nucleotides, or more than 50,000 nucleotides. In some embodiments, the expression cassette can comprise a transgene in the range of from about 500 to about 50,000 nucleotides in length. In some embodiments, the expression cassette can comprise a transgene in the range of from about 500 to about 75,000 nucleotides in length. In some embodiments, the expression cassette can comprise a transgene that is in the range of from about 500 to about 10,000 nucleotides in length. In some embodiments, the expression cassette can comprise a transgene that is in the range of from about 1000 to about 10,000 nucleotides in length. In some embodiments, the expression cassette can comprise a transgene that is in the range of from about 500 to about 5,000 nucleotides in length. In some embodiment, the DNA molecules provided herein do not have the size limitations of encapsidated AAV vectors, thus enabling delivery of a large-size expression cassette to provide efficient transgene. In certain embodiments, the DNA molecules provided herein comprise expression cassette equal to or larger than the size of any natural AAV genome.

The expression cassette can have various positions relative to the inverted repeat. In some embodiments, the expression cassette is at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or at least 100 nucleotides apart from the inverted repeat. In certain embodiments, the expression cassette is at least 0.2 kb, at least 0.3 kb, at least 0.4 kb, at least 0.5 kb, at least 0.6, at least kb, at least 0.7 kb, at least 0.8 kb, at least 0.9 kb, at least 1 kb, at least 1.5 kb, or at least 2 kb apart from the inverted repeat. In other embodiments, the expression cassette is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, or about 100 nucleotides apart from the inverted repeat. In further embodiments, the expression cassette is about 0.2 kb, about 0.3 kb, about 0.4 kb, about 0.5 kb, about 0.6, about kb, about 0.7 kb, about 0.8 kb, about 0.9 kb, about 1 kb, about 1.5 kb, or about 2 kb apart from the inverted repeat. In one embodiment, the inverted repeat in this paragraph is the first inverted repeat as described in Sections 3 and 5.4 (including 5.4.1). In another embodiment, the inverted repeat in this paragraph is the second inverted repeat as described in Sections 3 and 5.4 (including 5.4.1) In yet another embodiment, the inverted repeat in this paragraph is both the first and the second inverted repeat as described in Sections 3 and 5.4 (including 5.4.1).

As described above this Section (Section 5.4.3), the expression cassette can comprise one or more ORFs. In one embodiment, the ORF is an ORF of a human gene wherein genetic mutations in the human gene are known to cause a disease. In another embodiment, the ORF is an ORF of a human gene wherein genetic mutations in the human gene are known to cause a hereditary disease. In yet another embodiment, the ORF encodes a therapeutic protein. In a further embodiment, the ORF encodes an enzyme. In certain embodiments, the ORF encodes a metabolic enzyme. In some embodiments, the ORF encodes an enzyme, wherein the enzyme replaces or supplements the function of a defective enzyme in human. In one embodiment, the ORF encodes an antibody. In another embodiment, the ORF encodes a therapeutic antibody. In a further embodiment, the ORF encodes a cytokine. In yet another embodiment, the ORF encodes an RNA. In one embodiment, the ORF encodes a regulatory RNA. In another embodiment, the ORF encodes an anti-sense RNA. In yet another embodiment, the ORF encodes a siRNA. In a further embodiment, the ORF encodes a shRNA. In one embodiment, the ORF encodes a miRNA. In another embodiment, the ORF encodes a piRNA (PIWI-interacting RNA). In some embodiments, the expression cassette comprises any one or more features described in this Section (Section 5.4.3), in various permutations and combinations.

The various embodiments described in this Section (Section 5.4.3) with nicking endonucleases and/or restriction sites for nicking endonucleases are additionally provided with nicking endonucleases replaced by programmable nicking enzyme and restriction sites replaced by targeting sites for programmable nicking enzyme. The programmable nicking enzymes and their targeting sites for this paragraph and this Section (Section 5.4.3) have been provided in Section 5.3.4.

### 5.4.4 Hairpin-Ended DNA Molecules Encoding FVIII

Provided herein are hairpin-ended DNA molecules encoding a human Coagulation Factor VIII or functional fragment thereof.

In one aspect, provided herein is a double-stranded DNA molecule comprising in 5′ to 3′ direction of the sense strand: i) a first inverted repeat (e.g. as described in Section 5.4.1), wherein a first and a second restriction site for nicking endonuclease are arranged on opposite strands in proximity of the first inverted repeat such that nicking results in a sense strand 5′ overhang comprising the first inverted repeat upon separation of the sense from the antisense strand of the first inverted repeat (e.g. as described in Sections 5.3.3, 5.3.4 and 5.4.2); ii) a sense expression cassette encoding a therapeutic FVIII protein; and iii) a second inverted repeat (e.g. as described in Section 5.4.1), wherein a third and a fourth restriction site for nicking endonuclease are arranged on opposite strands in proximity of the second inverted repeat such that nicking results in a sense strand 3′ overhang comprising the second inverted repeat upon separation of the top from the antisense strand of the second inverted repeat (e.g. as described in Sections 5.3.3, 5.3.4 and 5.4.2)

In another aspect, provided herein is a double strand DNA molecule comprising in 5′ to 3′ direction of the sense strand: i) a first inverted repeat (e.g. as described in Section 5.4.1), wherein a first and a second restriction site for nicking endonuclease are arranged on opposite strands in proximity of the first inverted repeat such that nicking results in an antisense strand 3′ overhang comprising the first inverted repeat upon separation of the sense from the antisense strand of the first inverted repeat (e.g. as described in Sections 5.3.3, 5.3.4 and 5.4.2); ii) a sense expression cassette encoding a therapeutic FVIII protein; and iii) a second inverted repeat (e.g. as described in Section 5.4.1), wherein a third and a fourth restriction site for nicking endonuclease are arranged on opposite strands in proximity of the second inverted repeat such that nicking results in an antisense strand 5′ overhang comprising the second inverted repeat upon separation of the sense from the antisense of the second inverted repeat

In yet another aspect, provided herein is a double-stranded DNA molecule comprising in 5′ to 3′ direction of the sense strand: i) a first inverted repeat (e.g. as described in Section 5.4.1), wherein a first and a second restriction site for nicking endonuclease are arranged on opposite strands in proximity of the first inverted repeat such that nicking results in a sense strand 5′ overhang comprising the first inverted repeat upon separation of the sense from the antisense strand of the first inverted repeat (e.g. as described in Sections 5.3.3, 5.3.4 and 5.4.2); ii) a sense expression cassette encoding a therapeutic FVIII protein; and iii) a second inverted repeat (e.g. as described in Section 5.4.1), wherein a third and a fourth restriction site for nicking endonuclease are arranged on opposite strands in proximity of the second inverted repeat such that nicking results in an antisense strand 5′ overhang comprising the second inverted repeat upon separation of the sense from the antisense strand of the second inverted repeat (e.g. as described in Sections 5.3.3, 5.3.4 and 5.4.2).

In a further aspect, provide herein is a double strand DNA molecule comprising in 5′ to 3′ direction of the sense strand: i) a first inverted repeat (e.g. as described in Section 5.4.1), wherein a first and a second restriction site for nicking endonuclease are arranged on opposite strands in proximity of the first inverted repeat such that nicking results in an antisense strand 3′ overhang comprising the first inverted repeat upon separation of the sense from the antisense strand of the first inverted repeat (e.g. as described in Sections 5.3.3, 5.3.4 and 5.4.2); ii) a sense expression cassette encoding a therapeutic FVIII protein; and iii) a second inverted repeat (e.g. as described in Section 5.4.1), wherein a third and a fourth restriction site for nicking endonuclease are arranged on opposite strands in proximity of the second inverted repeat such that nicking results in a sense strand 3′ overhang comprising the second inverted repeat upon separation of the sense from the antisense strand of the second inverted repeat (e.g. as described in Sections 5.3.3, 5.3.4 and 5.4.2 or depicted in FIGS. 2B and 2C).

In one aspect, provided herein is a double-stranded DNA molecule comprising in 5′ to 3′ direction of the sense strand: i) a first inverted repeat (e.g. as described in Section 5.4.1), wherein a first and a second target site for the guide nucleic acids for programmable nicking enzyme are arranged on opposite strands in proximity of the first inverted repeat such that nicking by programmable nicking enzyme results in a sense strand 5′ overhang comprising the first inverted repeat upon separation of the sense from the antisense strand of the first inverted repeat (e.g. as described in Sections 5.3.3, 5.3.4 and 5.4.2); ii) a sense expression cassette encoding a therapeutic FVIII protein; and iii) a second inverted repeat (e.g. as described in Section 5.4.1), wherein a third and a fourth target site for the guide nucleic acids for programmable nicking endonuclease are arranged on opposite strands in proximity of the second inverted repeat such that nicking results in a sense strand 3′ overhang comprising the second inverted repeat upon separation of the sense from the antisense strand of the second inverted repeat (e.g. as described in Sections 5.3.3, 5.3.4 and 5.4.2 or depicted in FIGS. 2B and 2C).).

In one aspect, provided herein is a double-stranded DNA molecule comprising in the 5′ to 3′ direction of the sense strand: i) a first inverted repeat (e.g. as described in Section 5.4.1), wherein a first and a second target site for the guide nucleic acids for programmable nicking enzyme are arranged on opposite strands in proximity of the first inverted repeat such that nicking by programmable nicking enzyme results in an antisense strand 3′ overhang comprising the first inverted repeat upon separation of the sense from the antisense strand of the first inverted repeat (e.g. as described in Sections 5.3.3, 5.3.4 and 5.4.2); ii) a sense expression cassette encoding a therapeutic FVIII protein; and iii) a second inverted repeat (e.g. as described in Section 5.4.1), wherein a third and a fourth target site for the guide nucleic acids for programmable nicking enzyme are arranged on opposite strands in proximity of the second inverted repeat such that nicking by programmable nicking enzyme results in an antisense strand 5′ overhang comprising the second inverted repeat upon separation of the sense from the antisense strand of the second inverted repeat (e.g. as described in Sections 5.3.3, 5.3.4 and 5.4.2).

In yet another aspect, provided herein is a double-stranded DNA molecule comprising in 5′ to 3′ direction of the sense strand: i) a first inverted repeat (e.g. as described in Section 5.4.1), wherein a first and a second target site for the guide nucleic acids for programmable nicking enzyme are arranged on opposite strands in proximity of the first inverted repeat such that nicking by programmable nicking enzyme results in a sense strand 5′ overhang comprising the first inverted repeat upon separation of the sense from the antisense strand of the first inverted repeat (e.g. as described in Sections 5.3.3, 5.3.4 and 5.4.2); ii) a sense expression cassette encoding a therapeutic FVIII protein; and iii) a second inverted repeat (e.g. as described in Section 5.4.1), wherein a third and a fourth target site for the guide nucleic acids for programmable nicking enzyme are arranged on opposite strands in proximity of the second inverted repeat such that nicking by programmable nicking enzyme results in an antisense strand 5′ overhang comprising the second inverted repeat upon separation of the sense from the antisense strand of the second inverted repeat (e.g. as described in Sections 5.3.3, 5.3.4 and 5.4.2).

In yet another aspect, provided herein is a double-stranded DNA molecule comprising in the 5′ to 3′ direction of the sense strand: i) a first inverted repeat (e.g. as described in Section 5.4.1), wherein a first and a second target site for the guide nucleic acids for programmable nicking enzyme are arranged on opposite strands in proximity of the first inverted repeat such that nicking by programmable nicking enzyme results in a sense strand 5′ overhang comprising the first inverted repeat upon separation of the sense from the antisense strand of the first inverted repeat (e.g. as described in Sections 5.3.3, 5.3.4 and 5.4.2); ii) a sense expression cassette encoding a therapeutic FVIII protein; and iii) a second inverted repeat (e.g. as described in Section 5.4.1), wherein a third and a fourth target site for the guide nucleic acids for programmable nicking enzyme are arranged on opposite strands in proximity of the second inverted repeat such that nicking by programmable nicking enzyme results in an antisense strand 5′ overhang comprising the second inverted repeat upon separation of the sense from the antisense strand of the second inverted repeat (e.g. as described in Sections 5.3.3, 5.3.4 and 5.4.2).

In a further aspect, provide herein is a double strand DNA molecule comprising in 5′ to 3′ direction of the sense strand: i) a first inverted repeat (e.g. as described in Section 5.4.1), wherein a first and a second target site for the guide nucleic acids for programmable nicking enzyme are arranged on opposite strands in proximity of the first inverted repeat such that nicking by programmable nicking enzyme results in an antisense strand 3′ overhang comprising the first inverted repeat upon separation of the sense from the antisense strand of the first inverted repeat (e.g. as described in Sections 5.3.3, 5.3.4 and 5.4.2); ii) a sense expression cassette encoding a therapeutic FVIII protein; and iii) a second inverted repeat (e.g. as described in Section 5.4.1), wherein a third and a fourth target site for the guide nucleic acids for programmable nicking enzyme are arranged on opposite strands in proximity of the second inverted repeat such that nicking by programmable nicking enzyme results in a sense strand 3′ overhang comprising the second inverted repeat upon separation of the sense from the antisense strand of the second inverted repeat (e.g. as described in Sections 5.3.3, 5.3.4 and 5.4.2 or depicted in FIGS. 2B and 2C).

In one embodiment, the first, second, third, and fourth target site for programmable nicking enzyme in this and the preceding three paragraphs are all the same. In another embodiment, three of the first, second, third, and fourth target site for programmable nicking enzyme in this and the preceding three paragraphs are the same. In yet another embodiment, two of the first, second, third, and fourth target site for programmable nicking enzyme in this and the preceding three paragraphs are the same. In a further embodiment, the first, second, third, and fourth target site for programmable nicking enzyme in this and the preceding three paragraphs are all different.

### 5.4.5 Viral DNA Sequence Features Absent in the DNA Molecules Provided Herein

As further described in Sections 3, 5.2, 5.4.1, 5.4.2, 5.4.3, 5.4.6, 5.4.7 and 5.5, the DNA molecules provided can be produced either synthetically or recombinantly with or without certain sequence elements or features. As such, certain suitable and desired sequence features or elements can be included in the DNA molecules provided herein or excluded from the DNA molecules provided herein. The corresponding methods for making such DNA molecules including or excluding the sequence features or elements are also provided herein as described by applying the methods of 5.2 with the DNA molecules of 5.4, which can produce various DNA molecules described in 5.5.

As described in Sections 3, 5.4.1, 5.6, and 6, such DNA sequence elements or features that can be excluded from the DNA molecules provided herein can be a viral replication-associated protein binding sequence (“RABS”), which refers to a DNA sequence to which viral DNA replication-associated protein (“RAP”) or an isoform thereof, encoded by Parvoviridae genes Rep or/and NS1 can bind. A RABS refers to a nucleotide sequence that includes both the nucleotide sequence recognized by a Rep or NS1 protein (for replication of viral nucleic acid molecules). Such an RBS is also referred to as RBE (Rep-binding element). Rep can also bind to a nucleotide sequence which forms a small palindrome comprising a single tip of an internal hairpin within the ITR, thereby stabilizing the association between Rep and the ITR. Such an RBS is also referred to as RBE′. In other embodiments, the RABS is an NS1-binding element (“NSBE”) to which replication-associated viral protein NS1 can bind. In some embodiments, Rep can bind to a nucleotide sequence in the stem structure of the ITR (i.e., the nucleotide sequence recognized by a Rep or NS1 protein (for replication of viral nucleic acid molecules) and/or the site of specific interaction between the Rep and/or NS1 protein and the nucleotide sequence. A RABS can be a sequence of 5 nucleotides to 300 nucleotides. In some embodiments of the DNA molecules provided herein including those provided in this Section 5.4.5, the RABS can be a sequence of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, at least 315, at least 320, at least 325, at least 330, at least 335, at least 340, at least 345, at least 350, at least 355, at least 360, at least 365, at least 370, at least 375, at least 380, at least 385, at least 390, at least 395, or at least 400 nucleotides. In some other embodiments, the RABS can be a sequence of about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, or about 400 nucleotides. In some further embodiments, any embodiment of the DNA molecules lacking an RABS described in this paragraph can be combined with any methods or DNA molecules provided herein including those provided in Sections 3, 5.2, 5.4, 5.5, and 6.

Alternatively, the DNA molecules provided herein, including those in Sections 3, 5.2, 5.4, 5.5, and 6, can lack a functional RABS by functionally inactivating the RABS sequence present in the DNA molecules with mutations, insertions, and/or deletions (including partial deletions or truncations), such that the RABS can no longer serve as a recognition and/or binding site for the Rep protein or NS1 protein. As such, in some embodiments of the DNA molecules provided herein, including those in Sections 3, 5.2, 5.4, 5.5, and 6, the DNA molecule comprise a functionally inactivated RABS. Such functional inactivation can be assessed by measuring and comparing the binding between the Rep or NS1 protein and the DNA molecules comprising the functionally inactivated RABS with that between the Rep or NS1 proteins and a reference molecule comprising the wild type (wt) RBS or NSBE sequences (e.g. the same DNA molecule but with wt RBS or wt NSBE sequences). Such binding can be determined by any binding measurements known and used in the field of molecular biology, for example, chromatin immunoprecipitation (ChIP) assays, DNA electrophoretic mobility shift assay (EMSA), DNA pull-down assays, or Microplate capture and detection assays, as further described in Matthew J. Guille & G. Geoff Kneale, Molecular Biotechnology 8:35-52 (1997); Bipasha Dey et al., Mol Cell Biochem. 2012 June; 365 (1-2): 279-99, both of which are hereby incorporated in their entireties by reference. In one embodiment, the binding between the RAPs and the functionally inactivated RABS in the DNA molecule is at most 0.001%, at most 0.01%, at most 0.1%, at most 1%, at most 1.5%, at most 2%, at most 2.5%, at most 3%, at most 3.5, at most 4%, at most 4.5%, at most 5%, at most 5.5%, at most 6%, at most 6.5%, at most 7%, at most 7.5%, at most 8%, at most 8.5%, at most 9%, at most 9.5%, or at most 10%, compared to the binding between the RAPs and the wild type RBS or NSBE in a reference DNA molecule (e.g. the same DNA molecule but with a wild type RBS or NSBE sequence). In another embodiment, the binding between the RAPs and the functionally inactivated RABS in the DNA molecule is about 0.001%, about 0.01%, about 0.1%, about 1%, about 1.5%, about 2%, about 2.5%, about 3%, about 3.5, about 4%, about 4.5%, about 5%, about 5.5%, about 6%, about 6.5%, about 7%, about 7.5%, about 8%, about 8.5%, about 9%, about 9.5%, or about 10%, compared to the binding between the RAPs and the wild type RABS in a reference DNA molecule (e.g. the same DNA molecule but with a wt RBS or NSBE sequence). In yet another embodiment, the binding between the RAPs and the functionally inactivated RABS in the DNA molecule is 0.001%, 0.01%, 0.1%, 1%, 1.5%, 2%, 2.5%, 3%, 3.5, 4%, 4.5%, 5%, 5.5%, 6%, 6.5%, 7%, 7.5%, 8%, 8.5%, 9%, 9.5%, or 10%, compared to the binding between the RAPs and the wild type RABS in a reference DNA molecule (e.g. the same DNA molecule but with a wt RBS or NSBE sequence).

Furthermore, the DNA molecules provided herein, including those in Sections 3, 5.2, 5.4, 5.5, and 6, can lack a functional RAPs or viral capsid encoding sequence by functionally inactivating the Rep protein, NS1 or viral capsid encoding sequence present in the DNA molecules with mutations, insertions, and/or deletions (including partial deletions or truncations), such that the RAPs or viral capsid encoding sequence can no longer functionally express the Rep protein, NS1 protein or viral capsid protein. Such functional inactivating mutations, insertions, or deletions can be achieved, for example, by using mutations, insertions, and/or deletions to shift the open reading frame of Rep protein, NS1 protein or viral capsid encoding sequence, by using mutations, insertions, and/or deletions to remove the start codon, by using mutations, insertions, and/or deletions to remove the promoter or transcription initiation site, by using mutations, insertions, and/or deletions to remove the RNA polymerase binding sites, by using mutations, insertions, and/or deletions to remove the ribosome recognition or binding sites, or other means known and used in the field.

In one embodiment, the DNA molecule comprise an RBS inactivated by mutation. In one embodiment, the DNA molecule comprise an RBS inactivated by a mutation of 1, 2, 3, 4, 5, 6, 7, 8, 9, 10, 11, 12, 13, 14, 15, 16, 17, 18, 19, 20, 20, 21, 22, 23, 24, 25, 26, 27, 28, 29, 10, 31, 32, 33, 34, 35, 36, 37, 38, 39, or 40 nucleotides in the RBS. In another embodiment, the DNA molecule comprise an RBS inactivated by a mutation of 1%, 2%, 3%, 4%, 5%, 6%, 7%, 8%, 9%, 10%, 11%, 12%, 13%, 14%, 15%, 16%, 17%, 18%, 19%, 20%, 20%, 21%, 22%, 23%, 24%, 25%, 26%, 27%, 28%, 29%, 10%, 31%, 32%, 33%, 34%, 35%, 36%, 37%, 38%, 39%, or 40% of the nucleotides in the RBS. In a further embodiment, the DNA molecule comprise an RBS inactivated by a deletion of 1, 2, 3, 4, 5, 6, 7, 8, 9, 10, 11, 12, 13, 14, 15, 16, 17, 18, 19, 20, 20, 21, 22, 23, 24, 25, 26, 27, 28, 29, 10, 31, 32, 33, 34, 35, 36, 37, 38, 39, or 40 nucleotides in the RBS. In yet another embodiment, the DNA molecule comprise an RBS inactivated by a deletion of 1%, 2%, 3%, 4%, 5%, 6%, 7%, 8%, 9%, 10%, 11%, 12%, 13%, 14%, 15%, 16%, 17%, 18%, 19%, 20%, 20%, 21%, 22%, 23%, 24%, 25%, 26%, 27%, 28%, 29%, 10%, 31%, 32%, 33%, 34%, 35%, 36%, 37%, 38%, 39%, or 40% of the nucleotides in the RBS. In some embodiments, the deletion of the preceding sentence is an internal deletion, a deletion from the 5′ end, or a deletion from the 3′ end. In some embodiments, the deletion of this paragraph can be any combination of internal deletions, deletion from the 5′ end, and/or deletions from the 3′ end. In certain embodiments, the DNA molecule comprise an RBS inactivated by a deletion of the entire RBS sequences. In some additional embodiments, the DNA molecule comprise an RBS inactivated by a partial deletion of the RBS sequences.

In one embodiment, the DNA molecule comprise an NBSE inactivated by mutation. In one embodiment, the DNA molecule comprise an NSBE inactivated by a mutation of 1, 2, 3, 4, 5, 6, 7, 8, 9, 10, 11, 12, 13, 14, 15, 16, 17, 18, 19, 20, 20, 21, 22, 23, 24, 25, 26, 27, 28, 29, 10, 31, 32, 33, 34, 35, 36, 37, 38, 39, or 40 nucleotides in the NSBE. In another embodiment, the DNA molecule comprise an NSBE inactivated by a mutation of 1%, 2%, 3%, 4%, 5%, 6%, 7%, 8%, 9%, 10%, 11%, 12%, 13%, 14%, 15%, 16%, 17%, 18%, 19%, 20%, 20%, 21%, 22%, 23%, 24%, 25%, 26%, 27%, 28%, 29%, 10%, 31%, 32%, 33%, 34%, 35%, 36%, 37%, 38%, 39%, or 40% of the nucleotides in the NSBE. In a further embodiment, the DNA molecule comprise an NSBE inactivated by a deletion of 1, 2, 3, 4, 5, 6, 7, 8, 9, 10, 11, 12, 13, 14, 15, 16, 17, 18, 19, 20, 20, 21, 22, 23, 24, 25, 26, 27, 28, 29, 10, 31, 32, 33, 34, 35, 36, 37, 38, 39, or 40 nucleotides in the NSBE. In yet another embodiment, the DNA molecule comprise an NSBE inactivated by a deletion of 1%, 2%, 3%, 4%, 5%, 6%, 7%, 8%, 9%, 10%, 11%, 12%, 13%, 14%, 15%, 16%, 17%, 18%, 19%, 20%, 20%, 21%, 22%, 23%, 24%, 25%, 26%, 27%, 28%, 29%, 10%, 31%, 32%, 33%, 34%, 35%, 36%, 37%, 38%, 39%, or 40% of the nucleotides in the NSBE. In some embodiments, the deletion of the preceding sentence is an internal deletion, a deletion from the 5′ end, or a deletion from the 3′ end. In some embodiments, the deletion of this paragraph can be any combination of internal deletions, deletion from the 5′ end, and/or deletions from the 3′ end. In certain embodiments, the DNA molecule comprise an NSBE inactivated by a deletion of the entire NSBE sequences. In some additional embodiments, the DNA molecule comprise an NSBE inactivated by a partial deletion of the NSBE sequences.

Similarly, DNA sequence elements or features can be included or excluded from any specific regions of the DNA molecules provided herein (including Sections 5.4 and 5.5) or any specific regions of the DNA molecules used in the methods provided herein (including Section 5.2). In one embodiment, the DNA molecule lacks a Rep protein encoding sequence. In one embodiment, the DNA molecule lacks a NS1 protein encoding sequence. In another embodiment, the DNA molecule lacks a viral capsid protein encoding sequence. In some embodiments, the expression cassette lacks a Rep protein encoding sequence. In some embodiments, the expression cassette lacks a NS1 protein encoding sequence. In certain embodiments, the expression cassette lacks a viral capsid protein encoding sequence. In a further embodiment, the DNA molecule lacks an RABS. In yet another embodiment, the first inverted repeat lacks an RABS. In one embodiment, the second inverted repeat lacks an RABS. In another embodiment, the DNA sequence between the ITR closing base pair of the first inverted repeat and the ITR closing base pair of the second inverted repeat lacks an RABS. The lack of an RABS can be the lack of one RABS, the lack of two RABSs, the lack of more than two RABs, or the lack of any RABS. In one embodiment, the DNA molecule comprises a functionally inactivated Rep protein recognition sequence. In one embodiment, the DNA molecule comprises a functionally inactivated NS1 protein recognition sequence. In one embodiment, the DNA molecule comprises a functionally inactivated NS1 protein encoding sequence. In another embodiment, the DNA molecule comprises a functionally inactivated viral capsid protein encoding sequence. In some embodiments, the expression cassette comprises a functionally inactivated Rep protein recognition sequence. In some embodiments, the expression cassette comprises a functionally inactivated NS1 protein recognition sequence. In certain embodiments, the expression cassette comprises a functionally inactivated viral capsid protein encoding sequence. In a further embodiment, the DNA molecule comprises a functionally inactivated RABS. In yet another embodiment, the first inverted repeat comprises a functionally inactivated RABS. In one embodiment, the second inverted repeat comprises a functionally inactivated RABS. In another embodiment, the DNA sequence between the ITR closing base pair of the first inverted repeat and the ITR closing base pair of the second inverted repeat comprises a functionally inactivated RABS. It is contemplated that one, two, or more RABS or all RABSs can be functionally inactivated.

Additionally, DNA sequence elements or features can be functionally inactivated from any combination of any specific regions of the DNA molecules provided herein (including Sections 5.4 and 5.5) or any specific regions of the DNA molecules used in the methods provided herein (including Section 5.2). In one embodiment, the first inverted repeat comprises a functionally inactivated RABS, and the second inverted repeat comprises a functionally inactivated RABS. In another embodiment, the first inverted repeat comprises a functionally inactivated RABS and the DNA sequence between the ITR closing base pair of the first inverted repeat and the ITR closing base pair of the second inverted repeat comprises a functionally inactivated RABS. In a further embodiment, the second inverted repeat comprises a functionally inactivated RABS and the DNA sequence between the ITR closing base pair of the first inverted repeat and the ITR closing base pair of the second inverted repeat comprises a functionally inactivated RABS. In yet another embodiment, the first inverted repeat comprises a functionally inactivated RABS, the second inverted repeat comprises a functionally inactivated RABS and the DNA sequence between the ITR closing base pair of the first inverted repeat and the ITR closing base pair of the second inverted repeat comprises a functionally inactivated RABS. It is contemplated that one, two, or more RABS or all RABSs can be functionally inactivated. In yet another embodiment, the first inverted repeat comprises a functionally inactivated RABS, the second inverted repeat comprises a functionally inactivated RBS and the DNA sequence between the ITR closing base pair of the first inverted repeat and the ITR closing base pair of the second inverted repeat comprises a functionally inactivated RABS.

As described in Sections 3, 5.4.1, 5.6, and 6, such DNA sequence elements or features that can be excluded from the DNA molecules provided herein can be a terminal resolution site (“TRS”). A TRS refers to a nucleotide sequence in the inverted repeat of the DNA molecules that includes the nucleotide sequence recognized by a RAP (for replication of viral nucleic acid molecules), the site of specific interaction between the RAP and the nucleotide sequence, and the site of specific cleavage by the endonuclease activity of the RAP protein. Nucleotide sequences of the conserved sites of specific cleavage by the endonuclease activity of the RAP proteins can be determined by DNA nicking assay known and used in the field of molecular biology, for example, gel electrophoresis, fluorophore-based in vitro nicking assays, radioactive in vitro nicking assay, as further described in Xu P, et al 2019. Antimicrob Agents Chemother 63: e01879-18.; US20190203229A; both of which are hereby incorporated in their entireties by reference. In some embodiments a TRS can be a nucleotide sequence in the inverted repeat of the DNA molecules that includes the nucleotide sequence recognized by a Rep protein (for replication of viral nucleic acid molecules), the site of specific interaction between the Rep protein and the nucleotide sequence, and the site of specific cleavage by the endonuclease activity of the Rep protein. In one embodiment a TRS can be a nucleotide sequence in the inverted repeat of the DNA molecules that includes the nucleotide sequence recognized by a NS1 protein (for replication of viral nucleic acid molecules), the site of specific interaction between the NS1 protein and the nucleotide sequence, and the site of specific cleavage by the endonuclease activity of the NS1 protein. A TRS can be a sequence of 5 nucleotides to 300 nucleotides. In some embodiments of the methods provided herein including those provided in this Section 5.4.5, the TRS can be a sequence of at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, at least 180, at least 185, at least 190, at least 195, at least 200, at least 205, at least 210, at least 215, at least 220, at least 225, at least 230, at least 235, at least 240, at least 245, at least 250, at least 255, at least 260, at least 265, at least 270, at least 275, at least 280, at least 285, at least 290, at least 295, at least 300, at least 305, at least 310, at least 315, at least 320, at least 325, at least 330, at least 335, at least 340, at least 345, at least 350, at least 355, at least 360, at least 365, at least 370, at least 375, at least 380, at least 385, at least 390, at least 395, or at least 400 nucleotides. In some other embodiments, the TRS can be a sequence of about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, or about 400 nucleotides. In some further embodiments, any embodiment of the TRS described in this paragraph can be combined with any methods or DNA molecules provided herein including those provided in Sections 3, 5.2, 5.4, 5.5, and 6.

Alternatively, the DNA molecules provided herein, including those in Sections 3, 5.2, 5.4, 5.5, and 6, can lack a functional TRS by functionally inactivating the TRS sequence present in the DNA molecules with mutations, insertions, and/or deletions (including partial deletions or truncations), such that the TRS can no longer serve as a recognition and/or binding site for the RAP (i.e. Rep and NS1). As such, in some embodiments of the DNA molecules provided herein, including those in Sections 3, 5.2, 5.4, 5.5, and 6, the DNA molecule comprise a functionally inactivated TRS. Such functional inactivation can be assessed by measuring and comparing the binding between the RAP (i.e. Rep and NS1) and the DNA molecules comprising the functionally inactivated TRS with that between the RAP and a reference molecule comprising the wild type (wt) TRS sequences (e.g. the same DNA molecule but with a wt TRS sequence). Such binding can be determined by any binding measurements known and used in the field of molecular biology, for example, chromatin immunoprecipitation (ChIP) assays, DNA electrophoretic mobility shift assay (EMSA), DNA pull-down assays, or Microplate capture and detection assays, as further described in Matthew J. Guille & G. Geoff Kneale, Molecular Biotechnology 8:35-52 (1997); Bipasha Dey et al., Mol Cell Biochem. 2012 June; 365 (1-2): 279-99, both of which are hereby incorporated in their entireties by reference. In one embodiment, the binding between the RAP (i.e. Rep and NS1) and the functionally inactivated TRS in the DNA molecule is at most 0.001%, at most 0.01%, at most 0.1%, at most 1%, at most 1.5%, at most 2%, at most 2.5%, at most 3%, at most 3.5, at most 4%, at most 4.5%, at most 5%, at most 5.5%, at most 6%, at most 6.5%, at most 7%, at most 7.5%, at most 8%, at most 8.5%, at most 9%, at most 9.5%, or at most 10%, compared to the binding between the RAP (i.e. Rep and NS1) and the wild type TRS in a reference DNA molecule (e.g. the same DNA molecule but with a wt TRS sequence). In another embodiment, the binding between the RAP (i.e. Rep and NS1) and the functionally inactivated TRS in the DNA molecule is about 0.001%, about 0.01%, about 0.1%, about 1%, about 1.5%, about 2%, about 2.5%, about 3%, about 3.5, about 4%, about 4.5%, about 5%, about 5.5%, about 6%, about 6.5%, about 7%, about 7.5%, about 8%, about 8.5%, about 9%, about 9.5%, or about 10%, compared to the binding between the RAP (i.e. Rep and NS1) and the wild type TRS in a reference DNA molecule (e.g. the same DNA molecule but with a wt TRS sequence). In yet another embodiment, the binding between the RAP (i.e. Rep and NS1) and the functionally inactivated TRS in the DNA molecule is 0.001%, 0.01%, 0.1%, 1%, 1.5%, 2%, 2.5%, 3%, 3.5, 4%, 4.5%, 5%, 5.5%, 6%, 6.5%, 7%, 7.5%, 8%, 8.5%, 9%, 9.5%, or 10%, compared to the binding between the RAP (i.e. Rep and NS1) and the wild type TRS in a reference DNA molecule (e.g. the same DNA molecule but with a wt TRS sequence).

In one embodiment, the DNA molecule comprise a TRS inactivated by mutation. In one embodiment, the DNA molecule comprise a TRS inactivated by a mutation of 1, 2, 3, 4, 5, 6, 7, 8, 9, 10, 11, 12, 13, 14, 15, 16, 17, 18, 19, 20, 20, 21, 22, 23, 24, 25, 26, 27, 28, 29, 10, 31, 32, 33, 34, 35, 36, 37, 38, 39, or 40 nucleotides in the TRS. In another embodiment, the DNA molecule comprise a TRS inactivated by a mutation of 1%, 2%, 3%, 4%, 5%, 6%, 7%, 8%, 9%, 10%, 11%, 12%, 13%, 14%, 15%, 16%, 17%, 18%, 19%, 20%, 20%, 21%, 22%, 23%, 24%, 25%, 26%, 27%, 28%, 29%, 10%, 31%, 32%, 33%, 34%, 35%, 36%, 37%, 38%, 39%, or 40% of the nucleotides in the TRS. In a further embodiment, the DNA molecule comprise a TRS inactivated by a deletion of 1, 2, 3, 4, 5, 6, 7, 8, 9, 10, 11, 12, 13, 14, 15, 16, 17, 18, 19, 20, 20, 21, 22, 23, 24, 25, 26, 27, 28, 29, 10, 31, 32, 33, 34, 35, 36, 37, 38, 39, or 40 nucleotides in the TRS. In yet another embodiment, the DNA molecule comprise a TRS inactivated by a deletion of 1%, 2%, 3%, 4%, 5%, 6%, 7%, 8%, 9%, 10%, 11%, 12%, 13%, 14%, 15%, 16%, 17%, 18%, 19%, 20%, 20%, 21%, 22%, 23%, 24%, 25%, 26%, 27%, 28%, 29%, 10%, 31%, 32%, 33%, 34%, 35%, 36%, 37%, 38%, 39%, or 40% of the nucleotides in the TRS. In some embodiments, the deletion of the preceding sentence is an internal deletion, a deletion from the 5′ end, or a deletion from the 3′ end. In some embodiments, the deletion of this paragraph can be any combination of internal deletions, deletion from the 5′ end, and/or deletions from the 3′ end. In certain embodiments, the DNA molecule comprise a TRS inactivated by a deletion of the entire TRS sequences. In some additional embodiments, the DNA molecule comprise a TRS inactivated by a partial deletion of the TRS sequences.

Similarly, DNA sequence elements or features can be included or excluded from any specific regions of the DNA molecules provided herein (including Sections 5.4 and 5.5) or any specific regions of the DNA molecules used in the methods provided herein (including Section 5.2). In one embodiment, the DNA molecule lacks a TRS. In yet another embodiment, the first inverted repeat lacks a TRS. In another embodiment, the second inverted repeat lacks a TRS. In a further embodiment, the first inverted repeat lacks a TRS, and the second inverted repeat lacks a TRS.

Alternatively, TRS sequence elements or features can be functionally inactivated from any specific regions of the DNA molecules provided herein (including Sections 5.4 and 5.5) or any specific regions of the DNA molecules used in the methods provided herein (including Section 5.2). In one embodiment, the DNA molecule comprises a functionally inactivated TRS. In yet another embodiment, the first inverted repeat comprises a functionally inactivated TRS. In another embodiment, the second inverted repeat comprises a functionally inactivated TRS. In a further embodiment, the first inverted repeat comprises a functionally inactivated TRS, and the second inverted repeat comprises a functionally inactivated TRS.

In some specific embodiments, the RABS excluded or functionally inactivated in the DNA molecules provided herein can be any, or any combination of any number, or all of the RABS sequences listed in the following table.

TABLE 20
Exemplary RAPs

| | | SEQ |
|---|---|---|
| | Corresponding | ID |
| RAPs | RABS sequences | NO |
| Rep | GCGCGCTCGCTCGCTC | 363 |
| (AAV1, | | |
| 2, 7) | | |
| Rep | TGCGCACTCGCTCGCTC | 364 |
| (AAV3) | | |
| Rep | GCGCGCTCGCTCACTC | 365 |
| (AAV4) | | |
| Rep | GTTCGCTCGCTCGCTGGCTC | 366 |
| (AAV5) | | |
| NS1- | GCCGCCGG | 419 |
| NSBE1 | | |
| (B19V) | | |
| NS1- | GGCGGGAC | 420 |
| NSBE2 | | |
| (B19V) | | |
| NS1- | TTCCGGTACA | 367 |
| NSBE3 | | |
| (B19V) | | |

Further non-limiting examples of terminal repeats for DNA molecules lacking RBS sequences are exemplified in FIG. 3 as well as corresponding SEQ ID NOs: 3, 4, 5, 8, 9 and 10. In some specific embodiments, the RABS excluded or functionally inactivated in the DNA molecules provided herein can be any, or any combination of any number, or all of the RABS sequences listed in the following table.

TABLE 21
Exemplary RAPs

| | Correspond- | Reverse |
|---|---|---|
| RAPs | ing RABS | Complement |
| | sequences | RABS |
| Rep | GCTCGCTCGCTC | GAGCGAGCGAGC |
| (AAV1, | | |
| 2, 7) | | |
| Rep | GATCGCTCGCTC | GAGCGAGCGATC |
| (AAV3) | | |
| Rep | GCTCGCTCACTC | GAGTGAGTGAGC |
| (AAV4) | ACTC | GAGC |
| Rep | GCTCGCTCGCTG | GAGCCAGCGAGC |
| (AAV5) | GCTC | GAGC |
| NS1- | GCCGCCGG | CCGGCGGC |
| NSBE1 | | |
| (B19V) | | |
| NS1- | GGCGGGAC | GTCCCGCC |
| NSBE2 | | |
| (B19V) | | |
| NS1- | TTCCGGTACA | TGTACCGGAA |
| NSBE3 | | |
| (B19V) | | |

In one specific embodiment, the DNA molecules lack recognition sequences for any one, or any combination of any number, or all of the RAPs described in the Table of the preceding paragraph. In another specific embodiment, the DNA molecule comprises functionally inactivated sequences encoding for any one, or any combination of any number, or all of the RAPs described in the Table of the preceding paragraph. In another specific embodiment, the DNA molecules comprises functionally inactivated sequences encoding for any one, or any combination of any number, or all of the RAPs described in Table 20 and 21.

In another specific embodiment, the DNA molecule comprises functionally inactivated recognition sequences for any one, or any combination of any number, or all of the RAPs described in Table 20 and. In one specific embodiment, one or both hairpinned inverted repeats lack the RAPS recognition sequence:

(SEQ ID NO: 334)
GGCCACTCCCGAAGAGCGCGCTCGCTATCT
CACTGAGGCCGGGCGACCAAAGGTCGCCCG
ACGCCCGGGCTTTGCCCGGGCGGCCTCAGT
GAGATAGCGAGCGCGCTCTTCGGGAGTGGC
C

Further non-exhaustive examples of hairpinned inverted repeats that may be included in DNA molecules lacking at least one, or any, RBS sequences are exemplified in Example 10.

In other specific embodiments, the TRS excluded or functionally inactivated in the DNA molecules provided herein can be any, or any combination of any number, or all of the TRS sequences listed in Table 22.

TABLE 22
Exemplary RAPs

| RAP | Corresponding |
|---|---|
| (Virus) | TRS sequences |
| Rep(AAV1, | AGTTGG |
| AAV2, | (SEQ ID NO: 421) |
| AAV3, | |
| AAV4) | |
| Rep(AAV5) | AGTGTGGC |
| | (SEQ ID NO: 422) |
| NS1 (B19) | GACACC |
| | (SEQ ID NO: 423) |
| NS1 (HBOV) | CTATATCT |
| | (SEQ ID NO: 424) |
| NS1 (MVM) | CTWW/TCA |
| | (W = A/T) |
| | (SEQ ID NO: 425) |

As the methods provided herein do not need a viral replication step and the DNA molecules provide herein do not need to be produced or replicated in a virus life cycle, the disclosure provides and a person reading the disclosure would understand that the DNA molecules provide herein can lack various DNA sequences or features, including those sequences or features provided in this Section (Section 5.4.5). DNA molecules lacking RABS and/or TRS and DNA molecules comprising functionally inactivated RABS and/or functionally inactivated TRS as provided in this Section 5.4.5 provide at least a major advantage in that the DNA molecules would have no or significantly lower risk of mobilization or replication once administered to a patient when compared with DNA molecules including such RABS and/or TRS sequences. Risk of mobilization or mobilization risk refers to the risk of the replication defective DNA molecules reverting to replication or production of viral particles in the host that has been administered the DNA molecules. Such mobilization risk can result from the presence of viral proteins (e.g. Rep proteins, NS1 proteins or viral capsid proteins) expressed by viruses that have infected the same host that has been administered the DNA molecules. Mobilization risk poses a significant safety concern for using the replication defective viral genome as gene therapy vectors, as described for example in Liujiang Song, Hum Gene Ther, 2020 October; 31 (19-20): 1054-1067 (incorporated herein in its entirety by reference). Such DNA molecules lacking RABS and/or TRS would have no binding site for viral RAP to initiate the replication even if other helper viruses are present in the same host to provide RAPs. Without being bound by theory, it is thought that presence replication initiation may require helper factors, which are provided by coinfections of the host by auxiliary viruses, referred to “helper viruses” which can include viruses from the herpesvirus family, adenoviruses, and papillomaviruses.

Accordingly, in some embodiments of the DNA molecules provided herein including those in this Section 5.4.5, the DNA molecules without RABS and/or without TRS have less mobilization risk after administered to a subject or a patient when compared with DNA molecules with RABS and/or with TRS. In certain embodiments of the DNA molecules provided herein including those in this Section 5.4.5, the DNA molecules comprising functionally inactivated RABS and/or functionally inactivated TRS have less mobilization risk after administered to a subject or a patient when compared with DNA molecules with RABS and/or with TRS. Such reduction of mobilization risk can be determined as (Pm−Po)/Pm, wherein Pm is the number of viral particles produced from the control DNA molecules with RABS when RAPs are present (e.g. due to the infection of any virus comprising RAPs or engineered expression of RAPs in the same host); Po is the number of viral particles produced from DNA molecules lacking RABS or comprising functionally inactivated as provided herein under comparable conditions in the same host used for the control DNA molecules. Alternatively, such reduction of mobilization risk can be determined as (Pm−Po)/Pm, wherein Pm is the number of viral particles produced from the control DNA molecules with TRS when RAPs are present (e.g. due to the infection of any virus comprising RAPs or engineered expression of RAPs in the same host); Po is the number of viral particles produced from DNA molecules lacking TRS or comprising functionally inactivated TRS as provided herein under comparable conditions in the same host used for the control DNA molecules. Additionally, such reduction of mobilization risk can be determined as (Pm−Po)/Pm, wherein Pm is the number of viral particles produced from the control DNA molecules with RABS and with TRS when RAPs are present (e.g. due to the infection of any virus comprising Rep proteins or NS1 proteins or engineered expression of Rep proteins or NS1 proteins in the same host); Po is the number of viral particles produced from DNA molecules (i) lacking RABS or comprising functionally inactivated RABS and (ii) lacking TRS or comprising functionally inactivated TRS as provided herein under comparable conditions in the same host used for the control DNA molecules. As described in Liujiang Song, Hum Gene Ther, 2020 October; 31 (19-20): 1054-1067 (incorporated herein in its entirety by reference), the host used for determining the particle numbers produced can be cells, animals (e.g. mouse, hamster, rate, dog, rabbit, guinea pig, and other suitable mammals), or human. The disclosure further provides and a person of ordinary skill in the art reading the disclosure would understand that Pm and Po, each as described in this paragraph, can be used also to determine the absolute or relative levels of mobilization. Briefly, in such an assay, the DNA molecules are transfected into the host cells (e.g. HEK293 cells) or transduced into the host cells by infecting with a viral particle comprising DNA molecules. The host cells are further transfected with Rep protein, NS1 protein or co-infected with another virus expressing the Rep protein or NS1 protein (for example wild type viruses). The host cells are then cultured to produce and release viral particles. Virions are then harvested by collecting both the host cell and the culture media after culturing 48 to 72 hours (e.g. 65 hours). The titer for the viral particles (proxy for Pm and Po) can be determined by a probe-based quantitative PCR (qPCR) analysis following Benzonase treatment to eliminate nonencapsidated DNA, as described in Song et al., Cytotherapy 2013; 15:986-998, which is incorporated in its entirety by reference. An exemplary implementation of such assay is provided in Liujiang Song, Hum Gene Ther, 2020 October; 31 (19-20): 1054-1067, which is incorporated herein in its entirety by reference.

Based on the determination of the reduction of mobilization risk and the mobilization risk levels, in some embodiments of the DNA molecules provided herein including in this Section 5.4.5, the mobilization risk of the DNA molecules when administered to a host is lower than control DNA molecules with RABS and/or with TRS by 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, or 20%. In certain embodiments, the mobilization risk of the DNA molecules when administered to a host is lower than control DNA molecules with RABS and/or with TRS by at least 99%, at least 98%, at least 97%, at least 96%, at least 95%, at least 94%, at least 93%, at least 92%, at least 91%, at least 90%, at least 89%, at least 88%, at least 87%, at least 86%, at least 85%, at least 84%, at least 83%, at least 82%, at least 81%, at least 80%, at least 79%, at least 78%, at least 77%, at least 76%, at least 75%, at least 74%, at least 73%, at least 72%, at least 71%, at least 70%, at least 69%, at least 68%, at least 67%, at least 66%, at least 65%, at least 64%, at least 63%, at least 62%, at least 61%, at least 60%, at least 59%, at least 58%, at least 57%, at least 56%, at least 55%, at least 54%, at least 53%, at least 52%, at least 51%, at least 50%, at least 49%, at least 48%, at least 47%, at least 46%, at least 45%, at least 44%, at least 43%, at least 42%, at least 41%, at least 40%, at least 39%, at least 38%, at least 37%, at least 36%, at least 35%, at least 34%, at least 33%, at least 32%, at least 31%, at least 30%, at least 29%, at least 28%, at least 27%, at least 26%, at least 25%, at least 24%, at least 23%, at least 22%, at least 21%, or at least 20. In other embodiments, the mobilization risk of the DNA molecules when administered to a host is lower than control DNA molecules with RABS and/or with TRS by about 100%, about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, about 60%, about 59%, about 58%, about 57%, about 56%, about 55%, about 54%, about 53%, about 52%, about 51%, about 50%, about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, or about 20%.

Alternatively, in one embodiment, the DNA molecules provided herein including in this Section 5.4.5, result in no detectable mobilization (e.g. based on the measurement of Po provided in this Section 5.4.5). In another embodiment, the DNA molecules provided herein including in this Section 5.4.5 result in mobilization of no more than 0.0001%, no more than 0.001%, no more than 0.01%, no more than 0.1%, no more than 1%, no more than 1.5%, no more than 2%, no more than 2.5%, no more than 3%, no more than 3.5, no more than 4%, no more than 4.5%, no more than 5%, no more than 5.5%, no more than 6%, no more than 6.5%, no more than 7%, no more than 7.5%, no more than 8%, no more than 8.5%, no more than 9%, no more than 9.5%, or no more than 10%, of the mobilization resulted from a reference DNA molecule (e.g. the same DNA molecule but with a wild type RABS and/or with wild type TRS sequence). In a further embodiment, the DNA molecules provided herein including in this Section 5.4.5 result in mobilization of about 0.0001%, about 0.001%, about 0.01%, about 0.1%, about 1%, about 1.5%, about 2%, about 2.5%, about 3%, about 3.5, about 4%, about 4.5%, about 5%, about 5.5%, about 6%, about 6.5%, about 7%, about 7.5%, about 8%, about 8.5%, about 9%, about 9.5%, or about 10%, of the mobilization resulted from a reference DNA molecule (e.g. the same DNA molecule but with a wild type RABS and/or with wild type TRS sequence). In a yet another embodiment, the DNA molecules provided herein including in this Section 5.4.5 result in mobilization of 0.0001%, 0.001%, 0.01%, 0.1%, 1%, 1.5%, 2%, 2.5%, 3%, 3.5, 4%, 4.5%, 5%, 5.5%, 6%, 6.5%, 7%, 7.5%, 8%, 8.5%, 9%, 9.5%, or 10%, of the mobilization resulted from a reference DNA molecule (e.g. the same DNA molecule but with a wild type RABS and/or with wild type TRS sequence). Such percentage of mobilization can be determined by using the Pm and Po determined as further described in the preceding paragraphs (including the preceding 2 paragraphs).

In certain embodiments, the DNA molecules provided herein (for example, as in this Section 5.4.5) comprise ITRs that have functionally inactivated RABS and/or functionally inactivated TRS. In certain embodiments, the DNA molecules provided herein comprise ITRs that lack an RABS and/or a TRS. In certain embodiments, the DNA molecules provided herein have less mobilization risk after being administered to a subject or a patient when compared with DNA molecules comprising a functional RABS and/or functional TRS. ITRs that have functionally inactivated RABS and/or functionally inactivated TRS are referred to as “viral replication deficient inverted repeats” or “viral replication deficient inverted terminal repeats”, interchangeably.

In certain embodiments, the methods provided herein do not require any RABS. In certain embodiments, the DNA molecules provided herein do not need to be produced and/or replicated in a virus life cycle. The person of skill in the art would understand that the DNA molecules provided herein can lack additional features traditionally associated with RABS and/or viral production or replication, including those sequences or features discussed, for example, in this Section 5.4.5. In certain embodiments, the DNA molecules lacking RBS and/or DNA molecules comprising functionally inactivated RBS provided herein (for example, as in Section 5.4.5) provide at least a further advantage in that the terminal repeat sequences of DNA molecules may have no or diminished endogenous promoter and/or transcriptional activity (e.g. the P5 AAV promoter, which shares a homolog sequence with the RBS) once in a host cell when compared with DNA molecules with wild type viral ITR sequences. Transcriptional activity or endogenous promoter activity refers to the ability of hairpin ended DNA molecules to promote transgene expression starting from the folded hairpin overhang sequence (e.g. when these sequences contain one or more transcription start sites (TSSs)). Such transcriptional activity can result from the presence of viral proteins (e.g. Rep proteins or NS1 proteins) expressed by viruses that have infected the same host that has been administered the DNA molecules or from binding of endogenous transcription factors expressed in the host cell. The presence of TSSs and promoter sequences or fragments there of (e.g. the P5 promoter) may confound intended transgene expression in therapeutic applications or influence on transgene expression cassettes independent of promoter selection, wherein tight control of (e.g. tissue specific) transgene expression by appropriate control elements (e.g. tissue specific promoters) is highly desirable. In some embodiments, the presence or level of transcriptional activity and/or endogenous promoter activity arising from the folded hairpin overhang DNA sequence of hairpin ended DNA molecules (referred to as “ITR transcriptional activity”) can be determined by measuring the ability of such sequences to promote transgene expression in a host cell (e.g. by detecting report gene expression, qPCR of mRNA transcripts, western blot, etc.) by hairpin ended DNA molecules provided herein that lack a cis-regulatory element (e.g. promoter as described in section in 5.4.3) upstream of the ORF (e.g. by deleting or inactivating the promoter sequence of an expression cassette as described in 5.4.3).

In further embodiments, ITR transcriptional activity can be determined by measuring the residual ability of hairpin ended DNA molecules comprising an expression cassette comprising a tissue specific cis-regulatory element (e.g. tissue specific promoter as described in section in 5.4.3), to promote transgene expression in a host cell not derived from said tissue (e.g. by detecting report genes expression, qPCR of mRNA transcripts, western blot, etc.).

Based on the determination of the reduction ITR transcriptional activity and the ITR transcriptional activity levels, in some embodiments of the DNA molecules provided herein including in this Section 5.4.5, the ITR transcriptional activity of the DNA molecules when administered to a host is lower than control DNA molecules with wild type viral ITRs and/or RABS by 100%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, or 20%. In certain embodiments, the ITR transcriptional activity of the DNA molecules when administered to a host is lower than control DNA molecules with RABS and/or with wild type viral ITRs by at least 99%, at least 98%, at least 97%, at least 96%, at least 95%, at least 94%, at least 93%, at least 92%, at least 91%, at least 90%, at least 89%, at least 88%, at least 87%, at least 86%, at least 85%, at least 84%, at least 83%, at least 82%, at least 81%, at least 80%, at least 79%, at least 78%, at least 77%, at least 76%, at least 75%, at least 74%, at least 73%, at least 72%, at least 71%, at least 70%, at least 69%, at least 68%, at least 67%, at least 66%, at least 65%, at least 64%, at least 63%, at least 62%, at least 61%, at least 60%, at least 59%, at least 58%, at least 57%, at least 56%, at least 55%, at least 54%, at least 53%, at least 52%, at least 51%, at least 50%, at least 49%, at least 48%, at least 47%, at least 46%, at least 45%, at least 44%, at least 43%, at least 42%, at least 41%, at least 40%, at least 39%, at least 38%, at least 37%, at least 36%, at least 35%, at least 34%, at least 33%, at least 32%, at least 31%, at least 30%, at least 29%, at least 28%, at least 27%, at least 26%, at least 25%, at least 24%, at least 23%, at least 22%, at least 21%, or at least 20. In other embodiments, the ITR transcriptional activity of the DNA molecules when administered to a host is lower than control DNA molecules with RABS and/or with wild type viral ITRs by about 100%, about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61%, about 60%, about 59%, about 58%, about 57%, about 56%, about 55%, about 54%, about 53%, about 52%, about 51%, about 50%, about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, or about 20%.

In a certain embodiment, the DNA molecules provided herein including in this Section 5.4.5, result in no detectable ITR transcriptional activity (e.g. based on the measurement of transgene expression method in this Section 5.4.5). In another embodiment, the DNA molecules provided herein including in this Section 5.4.5 result in ITR transcriptional activity of no more than 0.0001%, no more than 0.001%, no more than 0.01%, no more than 0.1%, no more than 1%, no more than 1.5%, no more than 2%, no more than 2.5%, no more than 3%, no more than 3.5, no more than 4%, no more than 4.5%, no more than 5%, no more than 5.5%, no more than 6%, no more than 6.5%, no more than 7%, no more than 7.5%, no more than 8%, no more than 8.5%, no more than 9%, no more than 9.5%, or no more than 10%, of the ITR transcriptional activity resulted from a reference DNA molecule (e.g. the same DNA molecule but with a wild type RABS and/or with wild type ITR sequence). In a further embodiment, the DNA molecules provided herein including in this Section 5.4.5 result in ITR transcriptional activity of about 0.0001%, about 0.001%, about 0.01%, about 0.1%, about 1%, about 1.5%, about 2%, about 2.5%, about 3%, about 3.5, about 4%, about 4.5%, about 5%, about 5.5%, about 6%, about 6.5%, about 7%, about 7.5%, about 8%, about 8.5%, about 9%, about 9.5%, or about 10%, of the ITR transcriptional activity resulted from a reference DNA molecule (e.g. the same DNA molecule but with a wild type RABS and/or with wild type ITR sequence). In a yet another embodiment, the DNA molecules provided herein including in this Section 5.4.5 result in ITR transcriptional activity of 0.0001%, 0.001%, 0.01%, 0.1%, 1%, 1.5%, 2%, 2.5%, 3%, 3.5, 4%, 4.5%, 5%, 5.5%, 6%, 6.5%, 7%, 7.5%, 8%, 8.5%, 9%, 9.5%, or 10%, of the mobilization ITR transcriptional activity from a reference DNA molecule (e.g. the same DNA molecule but with a wild type RABS and/or with wild type ITR sequence). Such percentage of ITR transcriptional activity can be determined by using the transgene expression determined as further described in the preceding paragraphs (including the preceding 2 paragraphs).

As is clear from the descriptions in this Section 5.4.5, the DNA sequences or features excluded in the DNA molecules provided herein can be combined in any way with any of the methods provided herein (including in Sections 3, 5.2, and 6), any of the DNA molecules provided herein (including Sections 3, 5.4, and 6), and any of the hairpin-ended DNA molecules provided herein (including Sections 3, 5.5, and 6), and contribute to the functional properties of the DNA molecules as provided herein (including Sections 3, 5.6, and 6).

### 5.4.6 DNA Vectors

The disclosure provides that the DNA molecules can be of various forms. In one embodiment, the DNA molecule provided for the methods and composition herein is a vector. A vector is a nucleic acid molecule that can be replicated and/or expressed in a host cell. Any vectors known to those skilled in the art are provided herein. In some embodiments, the vector can be plasmids, viral vectors, cosmids, and artificial chromosomes (e.g., bacterial artificial chromosomes or yeast artificial chromosomes). In one specific embodiment, the vector is a plasmid. In another specific embodiment, the vector is a double stranded DNA molecule. As is clear from the description, when the DNA molecules are in the form of a vector (including a plasmid), the vector would comprise all the features described herein for the DNA molecules, including those described in Section 3 and this Section (Section 5.4).

In some embodiments, the vector provided in this Section (Section 5.4.6) can be used for the production of DNA molecules provided in Sections 3 and 5.5, for example by performing the method steps provided in Section 5.2. As such, the vector provided in this Section (Section 5.4.6) (1) comprises the features of the DNA molecules provided in Sections 3 and 5.5, including IRs or ITRs that can form hairpins as described in Sections 5.4.1 and 5.5, expression cassette as described in 5.4.3, and restriction sites for nicking endonucleases or restriction enzymes as described in Sections 5.4.2, 5.3.4, and 5.4.7, and/or (2) lacks the RABS and/or TRS sequences as described in Section 5.4.5. Therefore, the disclosure provides that the vector provided in this Section (Section 5.4.6) can (1) comprise any combination of embodiments of IRs or ITRs that can form hairpins as described in Sections 5.4.1 and 5.5, expression cassette as described in 5.4.3, restriction sites for nicking endonucleases or restriction enzymes as described in Sections 5.4.2 5.3.4, and 5.4.7, and additional features for the vectors provided in this Section (Section 5.4.6), and/or (2) lacks the RABS and/or TRS sequences as described in Section 5.4.5. In some embodiments, a vector can be constructed using known techniques to provide at least the following as operatively linked components in the direction of transcription: (1) a 5′ ITR sequence; (2) an expression cassette comprising a cis-regulatory element, for example, a promoter, inducible promoter, regulatory switch, enhancers and the like; and (3) a 3′ IR sequence. In some embodiments, the expression cassette is flanked by the ITRs comprises a cloning site for introducing an exogenous sequence.

Specifically, in one embodiment, the DNA molecule is a plasmid. Plasmid is widely known and used in the art as a vector to replicate or express the DNA molecules in the plasmid. Plasmid often refers to a double-stranded and/or circular DNA molecule that is capable of autonomous replication in a suitable host cell. In certain embodiments, plasmids provided for the methods described in Section 5.2 can be linearized by restriction enzyme digest and/or be present in a linear form. In certain embodiments, plasmids provided for the methods described in Section 5.2 can be linearized by isothermal amplification and/or be present in a linear form. Plasmids provided for the methods and compositions described herein include commercially available plasmids for use in well-known host cells (including both prokaryotic and eukaryotic host cells), as available from various vendors and/or described in Molecular Cloning: A Laboratory Manual, 4th Edition, by Michael Green and Joseph Sambrook, ISBN 978-1-936113-42-2 (2012), which is incorporated herein in its entirety by reference. In certain embodiments, the plasmids further comprise a multiple cloning site. In some embodiments, the plasmids further comprise a selection marker, which for example, can be an antibiotic resistance gene. In some embodiments, the plasmids further comprise an origin of replication (ORI). An ORI is a sequence at which replication is initiated, enabling a plasmid to reproduce within the host cells. In certain embodiments, the ORI provided for the methods and compositions described herein can be a bacterial origin of replication. In certain embodiments, the ORI provided for the methods and compositions described herein can be a eukaryotic origin of replication. In certain embodiments, the ORI provided for the methods and compositions described herein can be a viral origin of replication. In some specific embodiments, the ORI can be pBR322, F1, ColE1, pMB1, pUC, pSC101, R6K, 15A, EBV ORI, or SV40 ORI.

The plasmids described in this Section (Section 5.4.6) can further comprise other features. In some embodiments, the plasmid further comprises a restriction enzyme site (e.g. restriction enzyme site as described in Sections 5.3.4 and 5.4.2) in the region 5′ to the first inverted repeat and 3′ to the second inverted repeat wherein the restriction enzyme site is not present in any of the first inverted repeat, second inverted repeat, and the region between the first and second inverted repeats. In certain embodiments, the cleavage with the restriction enzyme at the restriction site described in this paragraph results in single strand overhangs that do not anneal at detectable levels under conditions that favor annealing of the first and/or second inverted repeat (e.g. conditions as described in Section 5.3.5). In some other embodiments, the plasmid further comprises an open reading frame encoding the restriction enzyme recognizing and cleaving the restriction site describe in this paragraph. In certain embodiments, the restriction enzyme site and the corresponding restriction enzyme can be any one of the restriction enzyme site and its corresponding restriction enzyme described in Sections 5.3.4 and 5.4.2. In further embodiment, the expression of the restriction enzyme described in this paragraph is under the control of a promoter. In some embodiments, the promoter described in this paragraph can be any promoter described above in Section 5.4.3. In another embodiment, the promoter described is an inducible promoter. In certain embodiment, the inducible promoter is a chemically inducible promoter. In further embodiments, the inducible promoter is any one selected from the group consisting of: tetracycline ON (Tet-On) promoter, negative inducible pLac promoter, alcA, amyB, bli-3, bphA, catR, cbhl, cre1, exylA, gas, glaA, gla1, mir1, niiA, qa-2, Smxyl, tcu-1, thiA, vvd, xyl1, xyl1, xylP, xyn1, and ZeaR, as described in Janina Kluge et al., Applied Microbiology and Biotechnology 102:6357-6372 (2018), which is incorporated herein in its entirety by reference.

Similarly, in certain embodiments, the plasmid can further comprise a fifth and a sixth restriction site for nicking endonuclease (e.g. restriction site for nicking endonuclease as described in Sections 5.3.4 and 5.4.2) in the region 5′ to the first inverted repeat and 3′ to the second inverted repeat, wherein the fifth and sixth restriction sites for nicking endonuclease are: a.) on opposite strands; and b.) create a break in the double stranded DNA molecule such that the single strand overhangs of the break do not anneal at detectable levels inter- or intra-molecularly under conditions that favor annealing of the first and/or second inverted repeat (e.g. conditions as described in Section 5.3.5). As is clear from the description of Section 5.3.4, incubation with nicking endonucleases will result in a fifth nick corresponding to the fifth restriction site for the nicking endonuclease and a sixth nick corresponding to the sixth restriction site for the nicking endonuclease. The disclosure provides that the fifth and sixth nick can have various relative positions between them. In one embodiment, the fifth and the sixth nick are 1, 2, 3, 4, 5, 6, 7, 8, 9, 10, 11, 12, 13, 14, 15, 16, 17, 18, 19, or 20 nucleotides apart. In some embodiments, as the ssDNA overhang between fifth and sixth nick does not anneal at detectable levels inter- or intra-molecularly under conditions that favor annealing of the first and/or second inverted repeat, the ssDNA overhang resulted from fifth and sixth nick has a lower melting temperature than the ssDNA overhangs described in Sections 5.3.3 and 5.4.2. In certain embodiments, the ssDNA overhang resulted from fifth and sixth nick is shorter than the ssDNA overhangs described in Sections 5.3.3 and 5.4.2. In other embodiments, the ssDNA overhang resulted from fifth and sixth nick has a lower percentage of G-C content than the ssDNA overhangs described in Sections 5.3.3 and 5.4.2. In some specific embodiments, the ssDNA overhang resulted from fifth and sixth nick is 0, 1, 2, 3, 4, 5, 6, 7, 8, 9, 10, 11, 12, 13, 14, 15, 16, 17, 18, 19, or 20 nucleotides in length. In other specific embodiments, the ssDNA overhang resulted from fifth and sixth nick is shorter than the ssDNA overhangs described in Sections 5.3.3 and 5.4.2 by at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 105, at least 110, at least 115, at least 120, at least 125, at least 130, at least 135, at least 140, at least 145, at least 150, at least 155, at least 160, at least 165, at least 170, at least 175, or at least 180 nucleotides. In some specific embodiments, the ssDNA overhang resulted from fifth and sixth nick is shorter than the ssDNA overhangs described in Sections 5.3.3 and 5.4.2 by about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, or about 180 nucleotides.

In certain embodiments, the plasmid can further comprise 7, 8, 9, 10, 11, 12, 13, 14, 15, 16, 17, 18, 19 or more restriction sites for nicking endonuclease (e.g. restriction site for nicking endonuclease as described in Sections 5.3.4 and 5.4.2) in the region 5′ to the first inverted repeat and 3′ to the second inverted repeat, wherein the additional restriction sites for nicking endonuclease may be: a.) on opposite strands; and b.) create a break in the double stranded DNA molecule such that the single strand overhangs of the break do not anneal at detectable levels inter- or intra-molecularly under conditions that favor annealing of the first and/or second inverted repeat (e.g. conditions as described in Section 5.3.5). In certain embodiments, the nicks in the region 5′ to the first inverted repeat and 3′ to the second inverted repeat, can have various relative positions between them. In one embodiment, the nicks in the region 5′ to the first inverted repeat and 3′ to the second inverted repeat, are 1, 2, 3, 4, 5, 6, 7, 8, 9, 10, 11, 12, 13, 14, 15, 16, 17, 18, 19, or 20 nucleotides apart. In some embodiments, as the ssDNA overhang between the nicks in the region 5′ to the first inverted repeat and 3′ to the second inverted repeat does not anneal at detectable levels inter- or intra-molecularly under conditions that favor annealing of the first and/or second inverted repeat, the ssDNA overhang resulting from the nicks in the region 5′ to the first inverted repeat and 3′ to the second inverted repeat has a lower melting temperature than the ssDNA overhangs described in Sections 5.3.3 and 5.4.2. In certain embodiments, the ssDNA overhang resulting from the nicks in the region 5′ to the first inverted repeat and 3′ to the second inverted repeat is shorter than the ssDNA overhangs described in Sections 5.3.3 and 5.4.2. In other embodiments, the ssDNA overhang resulting from the nicks in the region 5′ to the first inverted repeat and 3′ to the second inverted repeat has a lower percentage of G-C content than the ssDNA overhangs described in Sections 5.3.3 and 5.4.2. In some specific embodiments, the ssDNA overhang resulting from the nicks in the region 5′ to the first inverted repeat and 3′ to the second inverted repeat is 0, 1, 2, 3, 4, 5, 6, 7, 8, 9, 10, 11, 12, 13, 14, 15, 16, 17, 18, 19, or 20 nucleotides in length.

As described above in Sections 5.3.4 and 5.4.2, in various embodiments, the first, second, third, and fourth restriction sites for nicking endonuclease can be the target sequences for the same or different nicking endonucleases. Similar, in certain embodiments, the fifth and sixth restriction sites for nicking endonuclease can be target sequences for the same or different nicking endonucleases. In some embodiments, the first, second, third, fourth, fifth, and sixth restriction sites for nicking endonuclease provided for the DNA molecules as described in Sections 3 and 5.3.4 and this Section 5.4 can be all for target sequences for the same nicking endonuclease. Alternatively, in other embodiments, the first, second, third, fourth, fifth, and sixth restriction sites for nicking endonucleases are target sequences for two different nicking endonucleases, including all possible combinations of arranging the six sites for two different nicking endonuclease target sequences (e.g. the first restriction site for the first nicking endonuclease and the rest for the second nicking endonuclease, the first and second restriction sites for the first nicking endonuclease and the rest for the second nicking endonuclease, etc.). Additionally, in certain embodiments, the first, second, third, fourth, fifth, and sixth restriction sites for nicking endonucleases are target sequences for three different nicking endonucleases, including all possible combinations of arranging the six sites for three different nicking endonuclease target sequences. Furthermore, in some embodiments, the first, second, third, fourth, fifth, and sixth restriction sites for nicking endonuclease are target sequences for four different nicking endonucleases, including all possible combinations of arranging the six sites for four different nicking endonuclease target sequences. Additionally, in some embodiments, the first, second, third, fourth, fifth, and sixth restriction sites for nicking endonuclease are target sequences for five different nicking endonucleases, including all possible combinations of arranging the six sites for five different nicking endonuclease target sequences. Furthermore, in some embodiments, the first, second, third, fourth, fifth, and sixth restriction sites for nicking endonuclease are target sequences for six different nicking endonucleases.

In some embodiments, the one or more of the nicking endonuclease sites described in the preceding paragraph are a target sequence of an endogenous nicking endonuclease. In some specific embodiments, the plasmid further comprises an ORF encoding a nicking endonuclease that recognizes one or more of the first, second, third, fourth, fifth, and sixth restriction sites for nicking endonuclease described in this Section (Section 5.4.6) including the preceding paragraph. In one specific embodiment, the plasmid further comprises two ORFs encoding two nicking endonucleases that recognize two or more of the first, second, third, fourth, fifth, and sixth restriction sites for nicking endonuclease described in this Section (Section 5.4.6) including the preceding paragraph. In another specific embodiment, the plasmid further comprises three ORFs encoding three nicking endonucleases that recognize three or more of the first, second, third, fourth, fifth, and sixth restriction sites for nicking endonuclease described in this Section (Section 5.4.6) including the preceding paragraph. In yet another specific embodiment, the plasmid further comprises four ORFs encoding four nicking endonucleases that recognize four or more of the first, second, third, fourth, fifth, and sixth restriction sites for nicking endonuclease described in this Section (Section 5.4.6) including the preceding paragraph. In yet another specific embodiment, the plasmid further comprises four ORFs encoding four nicking endonucleases that recognize four or more of the first, second, third, fourth, fifth, and sixth restriction sites for nicking endonuclease described in this Section (Section 5.4.6) including the preceding paragraph. In a further specific embodiment, the plasmid further comprises five ORFs encoding five nicking endonucleases that recognize five or more of the first, second, third, fourth, fifth, and sixth restriction sites for nicking endonuclease described in this Section (Section 5.4.6) including the preceding paragraph. In one specific embodiment, the plasmid further comprises six ORFs encoding six nicking endonucleases that each recognizes the first, second, third, fourth, fifth, and sixth restriction sites for nicking endonuclease described in this Section (Section 5.4.6) including the preceding paragraph. In certain embodiments, the expression of the one or more nicking endonucleases described in this paragraph is under the control of a promoter. In some embodiments, the expression of the one or more nicking endonucleases described in this paragraph is under the control of a promoter. In some embodiments, the expression of the one or more nicking endonucleases described in this paragraph is under the control of an inducible promoter. In some specific embodiments, the inducible promoter can be any inducible promoter described above in this Section (Section 5.4.6).

In some embodiments, the nicking endonuclease that recognizes the first, second, third, and/or fourth restriction site for nicking endonuclease can be any one described in Sections 3, 5.3.4 and 5.4.2. In certain specific embodiment, the nicking endonuclease that recognizes the first, second, third, and/or fourth restriction site for nicking endonuclease is Nt. BsmAI; Nt. BtsCI; N. ALwl; N. BstNBI; N. BspD6I; Nb. Mva1269I; Nb. BsrDI; Nt. BtsI; Nt. Bsal; Nt. Bpu10I; Nt. BsmBI; Nb. BbvCI; Nt. BbvCI; or Nt. BspQI. In some embodiments, the nicking endonuclease that recognizes the fifth and sixth restriction site for nicking endonuclease can be any one described in Sections 3, 5.3.4 and 5.4.2. In certain specific embodiment, the nicking endonuclease that recognizes the fifth and sixth restriction site for nicking endonuclease is Nt. BsmAI; Nt. BtsCI; N. ALwl; N. BstNBI; N. BspD6I; Nb. Mva1269I; Nb. BsrDI; Nt. BtsI; Nt. Bsal; Nt. Bpu10I; Nt. BsmBI; Nb. BbvCI; Nt. BbvCI; or Nt. BspQI.

Additionally, in some embodiments, a plasmid provided in this Section (Section 5.4.6) can comprise a selectable or selection marker for use in the production of the plasmid in bacterial cultures. In one embodiment, the selection marker can be inserted downstream (e.g. 3′) of the 3′ ITR sequence. In another embodiment, the selection marker can be inserted upstream (e.g., 5′) of the 5′ IR sequence. A plasmid provided in this Section (Section 5.4.6) can also comprise a selectable or selection marker in between the IRs for use in the production of stable expressing cell line. In one embodiment, the selection marker can be inserted upstream (e.g. 5′) of the 3′ ITR sequence. In another embodiment, the selection marker can be inserted downstream (e.g. 3′) of the 5′ ITR sequence. Embodiments of appropriate selection markers include those that confer drug resistance. In certain embodiments, selection markers can be a blasticidin S-resistance gene, kanamycin, geneticin, and the like. In a specific embodiment, the drug selection marker is a chloramphenicol-resistance gene. In some embodiments, the plasmid can further comprise an ORF encoding a selection marker. In certain embodiments, the selection marker is an antibiotics resistant gene. In some specific embodiments, the selection marker is one providing resistance against selection agent selected from the group consisting of: kanamycin, spectinomycin, streptomycin, ampicillin, carbenicillin, bleomycin, erythromycin, polymyxin b, tetracycline, chloramphenicol, blasticidin, g418/geneticin, hygromycin B, puromycin, and zeocin.

In further embodiments, the plasmid of this Section (Section 5.4.6) can comprise one or more copies of the DNA molecules described in the paragraphs between the headings of Sections 5.4 and 5.4.1. In one specific embodiment, the plasmid of this Section (Section 5.4.6) can comprise one copy of the DNA molecules described in the paragraphs between the headings of Sections 5.4 and 5.4.1. In another specific embodiment, the plasmid of this Section (Section 5.4.6) can comprise two copy of the DNA molecules described in the paragraphs between the headings of Sections 5.4 and 5.4.1. In yet another specific embodiment, the plasmid of this Section (Section 5.4.6) can comprise 1, 2, 3, 4, 5, 6, 7, 8, 9, or 10 copies of the DNA molecules described in the paragraphs between the headings of Sections 5.4 and 5.4.1.

In some embodiments, the DNA molecules for the methods and composition provided herein (e.g. as provided in Section 3 and this Section (Section 5.4)) can be linear, non-circular DNA molecules.

In some embodiments, a vector for the methods and composition provided herein comprises any one or more features described in this Section (Section 5.4.6), in various permutations and combinations. In certain embodiments, a plasmid for the methods and composition provided herein comprises any one or more features described in this Section (Section 5.4.6). In certain embodiments, a plasmid for the methods and composition provided herein comprises any one or more features described in this Section (Section 5.4.6), in various permutations and combinations.

The various embodiments described in this Section (Section 5.4.6) with nicking endonucleases and/or restriction sites for nicking endonucleases are additionally provided with nicking endonucleases replaced by programmable nicking enzyme and restriction sites replaced by targeting sites for programmable nicking enzyme. The programmable nicking enzymes and their targeting sites for this paragraph and this Section (Section 5.4.3) have been provided in Section 5.3.4.

### 5.4.7 DNA Molecules With Less Than 4 Restriction Sites for Nicking Endonucleases and DNA Molecules With Less Than 4 Target Sites for Programmable Nicking Enzymes

In one additional aspect, provided herein is a double-stranded DNA molecule comprising in 5′ to 3′ direction of the top strand: i) a first inverted repeat (e.g. as described in Section 5.4.1), wherein a first restriction site for nicking endonuclease and a first restriction site for restriction enzyme are arranged in the opposite ends and in proximity of the first inverted repeat such that nicking and restriction enzyme cleavage result in a top strand 5′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette encoding a human FVIII or a catalytically active fragment thereof (e.g. as described in Section 5.4.3); and iii) a second inverted repeat (e.g. as described in Section 5.4.1), wherein a second restriction site for nicking endonuclease and a second restriction site for restriction enzyme are arranged in the opposite ends and in proximity of the second inverted repeat such that nicking and restriction enzyme cleavage result in a top strand 3′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2), wherein the first restriction site for restriction enzyme is more distal to expression cassette than the first restriction site for nicking endonuclease and the second restriction site for restriction enzyme is more distal to expression cassette than the second restriction site for nicking endonuclease. In certain embodiments, the top strand 5′ overhang comprises the first inverted repeat. In certain embodiments, the top strand 3′ overhang comprises the second inverted repeat. In certain embodiments, the top strand 5′ overhang comprises the first inverted repeat and the top strand 3′ overhang comprises the second inverted repeat.

In another aspect, provided herein is a double strand DNA molecule comprising in 5′ to 3′ direction of the top strand: i) a first inverted repeat (e.g. as described in Section 5.4.1), wherein a first restriction site for nicking endonuclease and a first restriction site for restriction enzyme are arranged in the opposite ends and in proximity of the first inverted repeat such that nicking and restriction enzyme cleavage result in a bottom strand 3′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette encoding a human FVIII or a catalytically active fragment thereof (e.g. as described in Section 5.4.3); and iii) a second inverted repeat (e.g. as described in Section 5.4.1), wherein a second restriction site for nicking endonuclease and a second restriction site for restriction enzyme are arranged in the opposite ends and in proximity of the second inverted repeat such that nicking and restriction enzyme cleavage result in a bottom strand 5′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2), wherein the first restriction site for restriction enzyme is more distal to expression cassette than the first restriction site for nicking endonuclease and the second restriction site for restriction enzyme is more distal to expression cassette than the second restriction site for nicking endonuclease. In certain embodiments, the bottom strand 3′ overhang comprises the first inverted repeat. In certain embodiments, the bottom strand 5′ overhang comprises the second inverted repeat. In certain embodiments, the bottom strand 3′ overhang comprises the first inverted repeat and the bottom strand 5′ overhang comprises the second inverted repeat.

In yet another aspect, provided herein is a double-stranded DNA molecule comprising in 5′ to 3′ direction of the top strand: i) a first inverted repeat (e.g. as described in Section 5.4.1), wherein a first restriction site for nicking endonuclease and a first restriction site for restriction enzyme are arranged in the opposite ends and in proximity of the first inverted repeat such that nicking and restriction enzyme cleavage result in a top strand 5′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette encoding a human FVIII or a catalytically active fragment thereof (e.g. as described in Section 5.4.3); and iii) a second inverted repeat (e.g. as described in Section 5.4.1), wherein a second restriction site for nicking endonuclease and a second restriction site for restriction enzyme are arranged in the opposite ends and in proximity of the second inverted repeat such that nicking and restriction enzyme cleavage result in a bottom strand 5′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2), wherein the first restriction site for restriction enzyme is more distal to expression cassette than the first restriction site for nicking endonuclease and the second restriction site for restriction enzyme is more distal to expression cassette than the second restriction site for nicking endonuclease. In certain embodiments, the top strand 5′ overhang comprises the first inverted repeat. In certain embodiments, the bottom strand 5′ overhang comprises the second inverted repeat. In certain embodiments, the top strand 5′ overhang comprises the first inverted repeat and the bottom strand 5′ overhang comprises the second inverted repeat.

In a further aspect, provide herein is a double strand DNA molecule comprising in 5′ to 3′ direction of the top strand: i) a first inverted repeat (e.g. as described in Section 5.4.1), wherein a first restriction site for nicking endonuclease and a first restriction site for restriction enzyme are arranged in the opposite ends and in proximity of the first inverted repeat such that nicking and restriction enzyme cleavage result in a bottom strand 3′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette encoding a human FVIII or a catalytically active fragment thereof (e.g. as described in Section 5.4.3); and iii) a second inverted repeat (e.g. as described in Section 5.4.1), wherein a second restriction site for nicking endonuclease and a second restriction site for restriction enzyme are arranged in the opposite ends and in proximity of the second inverted repeat such that nicking and restriction enzyme cleavage result in a top strand 3′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2), wherein the first restriction site for restriction enzyme is more distal to expression cassette than the first restriction site for nicking endonuclease and the second restriction site for restriction enzyme is more distal to expression cassette than the second restriction site for nicking endonuclease. In certain embodiments, the bottom strand 3′ overhang comprises the first inverted repeat. In certain embodiments, the top strand 3′ overhang comprises the second inverted repeat. In certain embodiments, the bottom strand 3′ overhang comprises the first inverted repeat and the top strand 3′ overhang comprises the second inverted repeat.

Additionally, in one aspect, provided herein is a double-stranded DNA molecule comprising in 5′ to 3′ direction of the top strand: i) a first inverted repeat (e.g. as described in Section 5.4.1), wherein a first and a second restriction site for nicking endonuclease are arranged on opposite strands in proximity of the first inverted repeat such that nicking results in a top strand 5′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette encoding a human FVIII or a catalytically active fragment thereof (e.g. as described in Section 5.4.3); and iii) a second inverted repeat (e.g. as described in Section 5.4.1), wherein a third restriction site for nicking endonuclease and a first restriction site for restriction enzyme are arranged in the opposite ends and in proximity of the second inverted repeat such that nicking and restriction enzyme cleavage result in a top strand 3′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2), wherein the first restriction site for restriction enzyme is more distal to expression cassette than the third restriction site for nicking endonuclease. In certain embodiments, the top strand 5′ overhang comprises the first inverted repeat. In certain embodiments, the top strand 3′ overhang comprises the second inverted repeat. In certain embodiments, the top strand 5′ overhang comprises the first inverted repeat and the top strand 3′ overhang comprises the second inverted repeat.

In another aspect, provided herein is a double strand DNA molecule comprising in 5′ to 3′ direction of the top strand: i) a first inverted repeat (e.g. as described in Section 5.4.1), wherein a first and a second restriction site for nicking endonuclease are arranged on opposite strands in proximity of the first inverted repeat such that nicking results in a bottom strand 3′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette encoding a human FVIII or a catalytically active fragment thereof (e.g. as described in Section 5.4.3); and iii) a second inverted repeat (e.g. as described in Section 5.4.1), wherein a third restriction site for nicking endonuclease and a first restriction site for restriction enzyme are arranged in the opposite ends and in proximity of the second inverted repeat such that nicking and restriction enzyme cleavage result in a bottom strand 5′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2), wherein the first restriction site for restriction enzyme is more distal to expression cassette than the third restriction site for nicking endonuclease. In certain embodiments, the bottom strand 3′ overhang comprises the first inverted repeat. In certain embodiments, the bottom strand 5′ overhang comprises the second inverted repeat. In certain embodiments, the bottom strand 3′ overhang comprises the first inverted repeat and the bottom strand 5′ overhang comprises the second inverted repeat.

In yet another aspect, provided herein is a double-stranded DNA molecule comprising in 5′ to 3′ direction of the top strand: i) a first inverted repeat (e.g. as described in Section 5.4.1), wherein a first and a second restriction site for nicking endonuclease are arranged on opposite strands in proximity of the first inverted repeat such that nicking results in a top strand 5′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette encoding a human FVIII or a catalytically active fragment thereof (e.g. as described in Section 5.4.3); and iii) a second inverted repeat (e.g. as described in Section 5.4.1), wherein a third restriction site for nicking endonuclease and a first restriction site for restriction enzyme are arranged in the opposite ends and in proximity of the second inverted repeat such that nicking and restriction enzyme cleavage result in a bottom strand 5′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2), wherein the first restriction site for restriction enzyme is more distal to expression cassette than the third restriction site for nicking endonuclease. In certain embodiments, the top strand 5′ overhang comprises the first inverted repeat. In certain embodiments, the bottom strand 5′ overhang comprises the second inverted repeat. In certain embodiments, the top strand 5′ overhang comprises the first inverted repeat and the bottom strand 5′ overhang comprises the second inverted repeat.

In a further aspect, provide herein is a double strand DNA molecule comprising in 5′ to 3′ direction of the top strand: i) a first inverted repeat (e.g. as described in Section 5.4.1), wherein a first and a second restriction site for nicking endonuclease are arranged on opposite strands in proximity of the first inverted repeat such that nicking results in a bottom strand 3′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette encoding a human FVIII or a catalytically active fragment thereof (e.g. as described in Section 5.4.3); and iii) a second inverted repeat (e.g. as described in Section 5.4.1), wherein a third restriction site for nicking endonuclease and a first restriction site for restriction enzyme are arranged in the opposite ends and in proximity of the second inverted repeat such that nicking and restriction enzyme cleavage result in a top strand 3′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2), wherein the first restriction site for restriction enzyme is more distal to expression cassette than the third restriction site for nicking endonuclease. In certain embodiments, the bottom strand 3′ overhang comprises the first inverted repeat. In certain embodiments, the top strand 3′ overhang comprises the second inverted repeat. In certain embodiments, the bottom strand 3′ overhang comprises the first inverted repeat and the top strand 3′ overhang comprises the second inverted repeat.

Additionally, in one aspect, provided herein is a double-stranded DNA molecule comprising in 5′ to 3′ direction of the top strand: i) a first inverted repeat (e.g. as described in Section 5.4.1), wherein a first restriction site for nicking endonuclease and a first restriction site for restriction enzyme are arranged in the opposite ends and in proximity of the first inverted repeat such that nicking and restriction enzyme cleavage result in a top strand 5′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette encoding a human FVIII or a catalytically active fragment thereof (e.g. as described in Section 5.4.3); and iii) a second inverted repeat (e.g. as described in Section 5.4.1), wherein a second and a third restriction site for nicking endonuclease are arranged on opposite strands in proximity of the second inverted repeat such that nicking results in a top strand 3′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2), wherein the first restriction site for restriction enzyme is more distal to expression cassette than the first restriction site for nicking endonuclease. In certain embodiments, the top strand 5′ overhang comprises the first inverted repeat. In certain embodiments, the top strand 3′ overhang comprises the second inverted repeat. In certain embodiments, the top strand 5′ overhang comprises the first inverted repeat and the top strand 3′ overhang comprises the second inverted repeat.

In another aspect, provided herein is a double strand DNA molecule comprising in 5′ to 3′ direction of the top strand: i) a first inverted repeat (e.g. as described in Section 5.4.1), wherein a first restriction site for nicking endonuclease and a first restriction site for restriction enzyme are arranged in the opposite ends and in proximity of the first inverted repeat such that nicking and restriction enzyme cleavage result in a bottom strand 3′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette encoding a human FVIII or a catalytically active fragment thereof (e.g. as described in Section 5.4.3); and iii) a second inverted repeat (e.g. as described in Section 5.4.1), wherein a second and a third restriction site for nicking endonuclease are arranged on opposite strands in proximity of the second inverted repeat such that nicking results in a bottom strand 5′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2), wherein the first restriction site for restriction enzyme is more distal to expression cassette than the first restriction site for nicking endonuclease. In certain embodiments, the bottom strand 3′ overhang comprises the first inverted repeat. In certain embodiments, the bottom strand 5′ overhang comprises the second inverted repeat. In certain embodiments, the bottom strand 3′ overhang comprises the first inverted repeat and the bottom strand 5′ overhang comprises the second inverted repeat.

In yet another aspect, provided herein is a double-stranded DNA molecule comprising in 5′ to 3′ direction of the top strand: i) a first inverted repeat (e.g. as described in Section 5.4.1), wherein a first restriction site for nicking endonuclease and a first restriction site for restriction enzyme are arranged in the opposite ends and in proximity of the first inverted repeat such that nicking and restriction enzyme cleavage result in a top strand 5′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette encoding a human FVIII or a catalytically active fragment thereof (e.g. as described in Section 5.4.3); and iii) a second inverted repeat (e.g. as described in Section 5.4.1), wherein a second and a third restriction site for nicking endonuclease are arranged on opposite strands in proximity of the second inverted repeat such that nicking results in a bottom strand 5′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2), wherein the first restriction site for restriction enzyme is more distal to expression cassette than the first restriction site for nicking endonuclease. In certain embodiments, the top strand 5′ overhang comprises the first inverted repeat. In certain embodiments, the bottom strand 5′ overhang comprises the second inverted repeat. In certain embodiments, the top strand 5′ overhang comprises the first inverted repeat and the bottom strand 5′ overhang comprises the second inverted repeat.

In a further aspect, provide herein is a double strand DNA molecule comprising in 5′ to 3′ direction of the top strand: i) a first inverted repeat (e.g. as described in Section 5.4.1), wherein a first restriction site for nicking endonuclease and a first restriction site for restriction enzyme are arranged in the opposite ends and in proximity of the first inverted repeat such that nicking and restriction enzyme cleavage result in a bottom strand 3′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette encoding a human FVIII or a catalytically active fragment thereof (e.g. as described in Section 5.4.3); and iii) a second inverted repeat (e.g. as described in Section 5.4.1), wherein a second and a third restriction site for nicking endonuclease are arranged on opposite strands in proximity of the second inverted repeat such that nicking results in a top strand 3′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2), wherein the first restriction site for restriction enzyme is more distal to expression cassette than the first restriction site for nicking endonuclease. In certain embodiments, the bottom strand 3′ overhang comprises the first inverted repeat. In certain embodiments, the top strand 3′ overhang comprises the second inverted repeat. In certain embodiments, the bottom strand 3′ overhang comprises the first inverted repeat and the top strand 3′ overhang comprises the second inverted repeat.

In one additional aspect, provided herein is a double-stranded DNA molecule comprising in 5′ to 3′ direction of the top strand: i) a first inverted repeat (e.g. as described in Section 5.4.1), wherein a first target site for the guide nucleic acid for programmable nicking enzyme and a first restriction site for restriction enzyme are arranged in the opposite ends and in proximity of the first inverted repeat such that nicking by the programmable nicking enzyme and restriction enzyme cleavage result in a top strand 5′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette encoding a human FVIII or a catalytically active fragment thereof (e.g. as described in Section 5.4.3); and iii) a second inverted repeat (e.g. as described in Section 5.4.1), wherein a second target site for the guide nucleic acid for programmable nicking enzyme and a second restriction site for restriction enzyme are arranged in the opposite ends and in proximity of the second inverted repeat such that nicking by the programmable nicking enzyme and restriction enzyme cleavage result in a top strand 3′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2), wherein the first restriction site for restriction enzyme is more distal to expression cassette than the first target site for the guide nucleic acid for programmable nicking enzyme and the second restriction site for restriction enzyme is more distal to expression cassette than the second target site for the guide nucleic acid for programmable nicking enzyme. In certain embodiments, the top strand 5′ overhang comprises the first inverted repeat. In certain embodiments, the top strand 3′ overhang comprises the second inverted repeat. In certain embodiments, the top strand 5′ overhang comprises the first inverted repeat and the top strand 3′ overhang comprises the second inverted repeat.

In another aspect, provided herein is a double strand DNA molecule comprising in 5′ to 3′ direction of the top strand: i) a first inverted repeat (e.g. as described in Section 5.4.1), wherein a first target site for the guide nucleic acid for programmable nicking enzyme and a first restriction site for restriction enzyme are arranged in the opposite ends and in proximity of the first inverted repeat such that nicking by programmable nicking enzyme and restriction enzyme cleavage result in a bottom strand 3′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette encoding a human FVIII or a catalytically active fragment thereof (e.g. as described in Section 5.4.3); and iii) a second inverted repeat (e.g. as described in Section 5.4.1), wherein a second target site for the guide nucleic acid for programmable nicking enzyme and a second restriction site for restriction enzyme are arranged in the opposite ends and in proximity of the second inverted repeat such that nicking by programmable nicking enzyme and restriction enzyme cleavage result in a bottom strand 5′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2), wherein the first restriction site for restriction enzyme is more distal to expression cassette than the first target site for the guide nucleic acid for programmable nicking enzyme and the second restriction site for restriction enzyme is more distal to expression cassette than the second target site for the guide nucleic acid for programmable nicking enzyme. In certain embodiments, the bottom strand 3′ overhang comprises the first inverted repeat. In certain embodiments, the bottom strand 5′ overhang comprises the second inverted repeat. In certain embodiments, the bottom strand 3′ overhang comprises the first inverted repeat and the bottom strand 5′ overhang comprises the second inverted repeat.

In yet another aspect, provided herein is a double-stranded DNA molecule comprising in 5′ to 3′ direction of the top strand: i) a first inverted repeat (e.g. as described in Section 5.4.1), wherein a first target site for the guide nucleic acid for programmable nicking enzyme and a first restriction site for restriction enzyme are arranged in the opposite ends and in proximity of the first inverted repeat such that nicking by programmable nicking enzyme and restriction enzyme cleavage result in a top strand 5′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette encoding a human FVIII or a catalytically active fragment thereof (e.g. as described in Section 5.4.3); and iii) a second inverted repeat (e.g. as described in Section 5.4.1), wherein a second target site for the guide nucleic acid for programmable nicking enzyme and a second restriction site for restriction enzyme are arranged in the opposite ends and in proximity of the second inverted repeat such that nicking by programmable nicking enzyme and restriction enzyme cleavage result in a bottom strand 5′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2), wherein the first restriction site for restriction enzyme is more distal to expression cassette than the first target site for the guide nucleic acid for programmable nicking enzyme and the second restriction site for restriction enzyme is more distal to expression cassette than the second target site for the guide nucleic acid for programmable nicking enzyme. In certain embodiments, the top strand 5′ overhang comprises the first inverted repeat. In certain embodiments, the bottom strand 5′ overhang comprises the second inverted repeat. In certain embodiments, the top strand 5′ overhang comprises the first inverted repeat and the bottom strand 5′ overhang comprises the second inverted repeat.

In a further aspect, provide herein is a double strand DNA molecule comprising in 5′ to 3′ direction of the top strand: i) a first inverted repeat (e.g. as described in Section 5.4.1), wherein a first target site for the guide nucleic acid for programmable nicking enzyme and a first restriction site for restriction enzyme are arranged in the opposite ends and in proximity of the first inverted repeat such that nicking by programmable nicking enzyme and restriction enzyme cleavage result in a bottom strand 3′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette encoding a human FVIII or a catalytically active fragment thereof (e.g. as described in Section 5.4.3); and iii) a second inverted repeat (e.g. as described in Section 5.4.1), wherein a second target site for the guide nucleic acid for programmable nicking enzyme and a second restriction site for restriction enzyme are arranged in the opposite ends and in proximity of the second inverted repeat such that nicking by programmable nicking enzyme and restriction enzyme cleavage result in a top strand 3′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2), wherein the first restriction site for restriction enzyme is more distal to expression cassette than the first target site for the guide nucleic acid for programmable nicking enzyme and the second restriction site for restriction enzyme is more distal to expression cassette than the second target site for the guide nucleic acid for programmable nicking enzyme. In certain embodiments, the bottom strand 3′ overhang comprises the first inverted repeat. In certain embodiments, the top strand 3′ overhang comprises the second inverted repeat. In certain embodiments, the bottom strand 3′ overhang comprises the first inverted repeat and the top strand 3′ overhang comprises the second inverted repeat.

Additionally, in one aspect, provided herein is a double-stranded DNA molecule comprising in 5′ to 3′ direction of the top strand: i) a first inverted repeat (e.g. as described in Section 5.4.1), wherein a first and a second target site for the guide nucleic acids for programmable nicking enzyme are arranged on opposite strands in proximity of the first inverted repeat such that nicking by programmable nicking enzyme results in a top strand 5′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette encoding a human FVIII or a catalytically active fragment thereof (e.g. as described in Section 5.4.3); and iii) a second inverted repeat (e.g. as described in Section 5.4.1), wherein a third target site for the guide nucleic acid for programmable nicking enzyme and a first restriction site for restriction enzyme are arranged in the opposite ends and in proximity of the second inverted repeat such that nicking by programmable nicking enzyme and restriction enzyme cleavage result in a top strand 3′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2), wherein the first restriction site for restriction enzyme is more distal to expression cassette than the third target site for the guide nucleic acid for programmable nicking enzyme. In certain embodiments, the top strand 5′ overhang comprises the first inverted repeat. In certain embodiments, the top strand 3′ overhang comprises the second inverted repeat. In certain embodiments, the top 5′ overhang comprises the first inverted repeat and the top strand 3′ overhang comprises the second inverted repeat.

In another aspect, provided herein is a double strand DNA molecule comprising in 5′ to 3′ direction of the top strand: i) a first inverted repeat (e.g. as described in Section 5.4.1), wherein a first and a second target site for the guide nucleic acids for programmable nicking enzyme are arranged on opposite strands in proximity of the first inverted repeat such that nicking by programmable nicking enzyme results in a bottom strand 3′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette encoding a human FVIII or a catalytically active fragment thereof (e.g. as described in Section 5.4.3); and iii) a second inverted repeat (e.g. as described in Section 5.4.1), wherein a third target site for the guide nucleic acid for programmable nicking enzyme and a first restriction site for restriction enzyme are arranged in the opposite ends and in proximity of the second inverted repeat such that nicking by programmable nicking enzyme and restriction enzyme cleavage result in a bottom strand 5′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2), wherein the first restriction site for restriction enzyme is more distal to expression cassette than the third target site for the guide nucleic acid for programmable nicking enzyme. In certain embodiments, the bottom strand 3′ overhang comprises the first inverted repeat. In certain embodiments, the bottom strand 5′ overhang comprises the second inverted repeat. In certain embodiments, the bottom strand 3′ overhang comprises the first inverted repeat and the bottom strand 5′ overhang comprises the second inverted repeat.

In yet another aspect, provided herein is a double-stranded DNA molecule comprising in 5′ to 3′ direction of the top strand: i) a first inverted repeat (e.g. as described in Section 5.4.1), wherein a first and a second target site for the guide nucleic acids for programmable nicking enzyme are arranged on opposite strands in proximity of the first inverted repeat such that nicking by programmable nicking enzyme results in a top strand 5′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette encoding a human FVIII or a catalytically active fragment thereof (e.g. as described in Section 5.4.3); and iii) a second inverted repeat (e.g. as described in Section 5.4.1), wherein a third target site for the guide nucleic acid for programmable nicking enzyme and a first restriction site for restriction enzyme are arranged in the opposite ends and in proximity of the second inverted repeat such that nicking by programmable nicking enzyme and restriction enzyme cleavage result in a bottom strand 5′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2), wherein the first restriction site for restriction enzyme is more distal to expression cassette than the third target site for the guide nucleic acid for programmable nicking enzyme. In certain embodiments, the top strand 5′ overhang comprises the first inverted repeat. In certain embodiments, the bottom strand 5′ overhang comprises the second inverted repeat. In certain embodiments, the top strand 5′ overhang comprises the first inverted repeat and the bottom strand 5′ overhang comprises the second inverted repeat.

In a further aspect, provide herein is a double strand DNA molecule comprising in 5′ to 3′ direction of the top strand: i) a first inverted repeat (e.g. as described in Section 5.4.1), wherein a first and a second target site for the guide nucleic acids for programmable nicking enzyme are arranged on opposite strands in proximity of the first inverted repeat such that nicking by programmable nicking enzyme results in a bottom strand 3′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette encoding a human FVIII or a catalytically active fragment thereof (e.g. as described in Section 5.4.3); and iii) a second inverted repeat (e.g. as described in Section 5.4.1), wherein a third target site for the guide nucleic acid for programmable nicking enzyme and a first restriction site for restriction enzyme are arranged in the opposite ends and in proximity of the second inverted repeat such that nicking by programmable nicking enzyme and restriction enzyme cleavage result in a top strand 3′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2), wherein the first restriction site for restriction enzyme is more distal to expression cassette than the third target site for the guide nucleic acid for programmable nicking enzyme. In certain embodiments, the bottom strand 3′ overhang comprises the first inverted repeat. In certain embodiments, the top strand 3′ overhang comprises the second inverted repeat. In certain embodiments, the bottom strand 3′ overhang comprises the first inverted repeat and the top strand 3′ overhang comprises the second inverted repeat.

Additionally, in one aspect, provided herein is a double-stranded DNA molecule comprising in 5′ to 3′ direction of the top strand: i) a first inverted repeat (e.g. as described in Section 5.4.1), wherein a first target site for the guide nucleic acid for programmable nicking enzyme and a first restriction site for restriction enzyme are arranged in the opposite ends and in proximity of the first inverted repeat such that nicking by programmable nicking enzyme and restriction enzyme cleavage result in a top strand 5′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette encoding a human FVIII or a catalytically active fragment thereof (e.g. as described in Section 5.4.3); and iii) a second inverted repeat (e.g. as described in Section 5.4.1), wherein a second and a third target site for the guide nucleic acids for programmable nicking enzyme are arranged on opposite strands in proximity of the second inverted repeat such that nicking by programmable nicking enzyme results in a top strand 3′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2), wherein the first restriction site for restriction enzyme is more distal to expression cassette than the first target site for the guide nucleic acid for programmable nicking enzyme. In certain embodiments, the top strand 5′ overhang comprises the first inverted repeat. In certain embodiments, the top strand 3′ overhang comprises the second inverted repeat. In certain embodiments, the top strand 5′ overhang comprises the first inverted repeat and the top strand 3′ overhang comprises the second inverted repeat.

In another aspect, provided herein is a double strand DNA molecule comprising in 5′ to 3′ direction of the top strand: i) a first inverted repeat (e.g. as described in Section 5.4.1), wherein a first target site for the guide nucleic acid for programmable nicking enzyme and a first restriction site for restriction enzyme are arranged in the opposite ends and in proximity of the first inverted repeat such that nicking by programmable nicking enzyme and restriction enzyme cleavage result in a bottom strand 3′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette encoding a human FVIII or a catalytically active fragment thereof (e.g. as described in Section 5.4.3); and iii) a second inverted repeat (e.g. as described in Section 5.4.1), wherein a second and a third target site for the guide nucleic acids for programmable nicking enzyme are arranged on opposite strands in proximity of the second inverted repeat such that nicking by programmable nicking enzyme results in a bottom strand 5′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2), wherein the first restriction site for restriction enzyme is more distal to expression cassette than the first target site for the guide nucleic acid for programmable nicking enzyme. In certain embodiments, the bottom strand 3′ overhang comprises the first inverted repeat. In certain embodiments, the bottom strand 5′ overhang comprises the second inverted repeat. In certain embodiments, the bottom strand 3′ overhang comprises the first inverted repeat and the bottom strand 5′ overhang comprises the second inverted repeat.

In yet another aspect, provided herein is a double-stranded DNA molecule comprising in 5′ to 3′ direction of the top strand: i) a first inverted repeat (e.g. as described in Section 5.4.1), wherein a first target site for the guide nucleic acid for programmable nicking enzyme and a first restriction site for restriction enzyme are arranged in the opposite ends and in proximity of the first inverted repeat such that nicking by programmable nicking enzyme and restriction enzyme cleavage result in a top strand 5′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette encoding a human FVIII or a catalytically active fragment thereof (e.g. as described in Section 5.4.3); and iii) a second inverted repeat (e.g. as described in Section 5.4.1), wherein a second and a third target site for the guide nucleic acids for programmable nicking enzyme are arranged on opposite strands in proximity of the second inverted repeat such that nicking by programmable nicking enzyme results in a bottom strand 5′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2), wherein the first restriction site for restriction enzyme is more distal to expression cassette than the first target site for the guide nucleic acid for programmable nicking enzyme. In certain embodiments, the top strand 5′ overhang comprises the first inverted repeat. In certain embodiments, the bottom strand 5′ overhang comprises the second inverted repeat. In certain embodiments, the top strand 5′ overhang comprises the first inverted repeat and the bottom strand 5′ overhang comprises the second inverted repeat.

In a further aspect, provide herein is a double strand DNA molecule comprising in 5′ to 3′ direction of the top strand: i) a first inverted repeat (e.g. as described in Section 5.4.1), wherein a first target site for the guide nucleic acid for programmable nicking enzyme and a first restriction site for restriction enzyme are arranged in the opposite ends and in proximity of the first inverted repeat such that nicking by programmable nicking enzyme and restriction enzyme cleavage result in a bottom strand 3′ overhang comprising the first inverted repeat or a fragment thereof (e.g., at least 50%, 60%, 70%, 75%, 80%, 85%, 90%, 95%, or 99% of the first inverted repeat) upon separation of the top from the bottom strand of the first inverted repeat (e.g. as described in Sections 5.3.3, 5.3.4 and 5.4.2); ii) an expression cassette encoding a human FVIII or a catalytically active fragment thereof (e.g. as described in Section 5.4.3); and iii) a second inverted repeat (e.g. as described in Section 5.4.1), wherein a second and a third target site for the guide nucleic acids for programmable nicking enzyme are arranged on opposite strands in proximity of the second inverted repeat such that nicking by programmable nicking enzyme results in a top strand 3′ overhang comprising the second inverted repeat or a fragment thereof (e.g., at least 50%, 60%, 70%, 75%, 80%, 85%, 90%, 95%, or 99% of the second inverted repeat) upon separation of the top from the bottom strand of the second inverted repeat (e.g. as described in Sections 5.3.3, 5.3.4 and 5.4.2), wherein the first restriction site for restriction enzyme is more distal to expression cassette than the first target site for the guide nucleic acid for programmable nicking enzyme. In certain embodiments, the bottom strand 3′ overhang comprises the first inverted repeat. In certain embodiments, the top strand 3′ overhang comprises the second inverted repeat. In certain embodiments, the bottom strand 3′ overhang comprises the first inverted repeat and the top strand 3′ overhang comprises the second inverted repeat.

The DNA molecules provided in this Section (Section 5.4.7) comprise various features or have various embodiments as described in this Section (Section 5.4.7), which features and embodiments are further described in the various subsections below: the embodiments for the inverted repeats, including the first inverted repeat and/or the second inverted repeat, are described in Section 5.4.1, the embodiments for the restriction enzymes, nicking endonucleases, and their respective restriction sites are described in Section 5.4.2, the embodiments for the programmable nicking enzymes and their target sites are described in Section 5.3.4, the embodiments for the expression cassette are described in Section 5.4.3, and the embodiments for plasmids and vectors are described in Section 5.4.6. As such, the disclosure provides DNA molecules comprising any permutations and combinations of the various embodiments of DNA molecules and embodiments of features of the DNA molecules described herein.

The various embodiments described in this Section (Section 5.4.7) with nicking endonucleases are interchangeable with programmable nicking enzyme and restriction sites for nicking endonucleases are interchangeable with the target sites for programmable nicking enzyme. As such, additional embodiments of any combination resulted by replacing one or more elements of nicking endonucleases with programmable nicking enzyme and/or replacing one or more elements of restriction sites for nicking endonucleases with the target sites for programmable nicking enzyme are provided herein in this Section (Section 5.4.7). The programmable nicking enzymes and their targeting sites for this paragraph and this Section (Section 5.4.3) have been provided in Section 5.3.4.

### 5.4.8 Isolated DNA Molecules

One of the advantages of the methods and DNA molecules provided herein is the purity of the isolated DNA molecules produced in the methods and provided herein, because the DNA molecules provided herein are resistant to exonuclease or other DNA digestion enzymes and thus can be treated, as described in Section 5.3.6, with such exonuclease or DNA digestion enzymes to remove the DNA contaminants that are susceptible to such treatment. As already described in the paragraphs between the heading of Section 5.4 and the heading of Section 5.4.1, the DNA molecules provided herein including in Sections 3, 5.2, 5.4, 5.5, and 6 can be isolated DNA molecules of various purity. Furthermore, the disclosure provides and a person of ordinary skill in the art would understand that the DNA molecules provided herein including in Sections 3, 5.2, 5.4, 5.5, and 6 can be free of certain general DNA contaminants, free of certain specific DNA contaminants, or both free of certain general DNA contaminants and free of certain specific DNA contaminants.

Accordingly, in one embodiment, the isolated DNA molecules are free of fragments of the DNA molecules. In another embodiment, the isolated DNA molecules are free of nucleic acid contaminants that are not fragments of the DNA molecules. In a further embodiment, the isolated DNA molecules are free of baculoviral DNA. In one embodiment, the isolated DNA molecules are free of fragments of the DNA molecules and free of nucleic acid contaminants that are not fragments of the DNA molecules. In another embodiment, the isolated DNA molecules are free of fragments of the DNA molecules and free of baculoviral DNA. In a further embodiment, the isolated DNA molecules are free of baculoviral DNA and free of nucleic acid contaminants that are not fragments of the DNA molecules. In yet another embodiment, the isolated DNA molecules are free of fragments of the DNA molecules, free of baculoviral DNA, and free of nucleic acid contaminants that are not fragments of the DNA molecules.

Specifically, in one embodiment, the fragments of the DNA molecules are no more than 1%, no more than 2%, no more than 3%, no more than 4%, no more than 5%, no more than 6%, no more than 7%, no more than 8%, no more than 9%, no more than 10%, no more than 11%, no more than 12%, no more than 13%, no more than 14%, no more than 15%, no more than 16%, no more than 17%, no more than 18%, no more than 19%, no more than 20%, no more than 21%, no more than 22%, no more than 23%, no more than 24%, no more than 25%, no more than 26%, no more than 27%, no more than 28%, no more than 29%, no more than 30%, no more than 31%, no more than 32%, no more than 33%, no more than 34%, no more than 35%, no more than 36%, no more than 37%, no more than 38%, no more than 39%, no more than 40%, no more than 41%, no more than 42%, no more than 43%, no more than 44%, no more than 45%, no more than 46%, no more than 47%, no more than 48%, no more than 49%, or no more than 50% of the isolated DNA molecules. In another embodiment, the fragments of the DNA molecules are less than 1%, less than 2%, less than 3%, less than 4%, less than 5%, less than 6%, less than 7%, less than 8%, less than 9%, less than 10%, less than 11%, less than 12%, less than 13%, less than 14%, less than 15%, less than 16%, less than 17%, less than 18%, less than 19%, less than 20%, less than 21%, less than 22%, less than 23%, less than 24%, less than 25%, less than 26%, less than 27%, less than 28%, less than 29%, less than 30%, less than 31%, less than 32%, less than 33%, less than 34%, less than 35%, less than 36%, less than 37%, less than 38%, less than 39%, less than 40%, less than 41%, less than 42%, less than 43%, less than 44%, less than 45%, less than 46%, less than 47%, less than 48%, less than 49%, or less than 50% of the isolated DNA molecules. In yet another embodiment, the fragments of the DNA molecules are about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% of the isolated DNA molecules.

Additionally, in one embodiment, the nucleic acid contaminants that are not fragments of the DNA molecules are no more than 1%, no more than 2%, no more than 3%, no more than 4%, no more than 5%, no more than 6%, no more than 7%, no more than 8%, no more than 9%, no more than 10%, no more than 11%, no more than 12%, no more than 13%, no more than 14%, no more than 15%, no more than 16%, no more than 17%, no more than 18%, no more than 19%, no more than 20%, no more than 21%, no more than 22%, no more than 23%, no more than 24%, no more than 25%, no more than 26%, no more than 27%, no more than 28%, no more than 29%, no more than 30%, no more than 31%, no more than 32%, no more than 33%, no more than 34%, no more than 35%, no more than 36%, no more than 37%, no more than 38%, no more than 39%, no more than 40%, no more than 41%, no more than 42%, no more than 43%, no more than 44%, no more than 45%, no more than 46%, no more than 47%, no more than 48%, no more than 49%, or no more than 50% of the isolated DNA molecules. In another embodiment, the nucleic acid contaminants that are not fragments of the DNA molecules are less than 1%, less than 2%, less than 3%, less than 4%, less than 5%, less than 6%, less than 7%, less than 8%, less than 9%, less than 10%, less than 11%, less than 12%, less than 13%, less than 14%, less than 15%, less than 16%, less than 17%, less than 18%, less than 19%, less than 20%, less than 21%, less than 22%, less than 23%, less than 24%, less than 25%, less than 26%, less than 27%, less than 28%, less than 29%, less than 30%, less than 31%, less than 32%, less than 33%, less than 34%, less than 35%, less than 36%, less than 37%, less than 38%, less than 39%, less than 40%, less than 41%, less than 42%, less than 43%, less than 44%, less than 45%, less than 46%, less than 47%, less than 48%, less than 49%, or less than 50% of the isolated DNA molecules. In yet another embodiment, the nucleic acid contaminants that are not fragments of the DNA molecules are about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% of the isolated DNA molecules.

In addition, in one embodiment, the baculoviral DNA are no more than 1%, no more than 2%, no more than 3%, no more than 4%, no more than 5%, no more than 6%, no more than 7%, no more than 8%, no more than 9%, no more than 10%, no more than 11%, no more than 12%, no more than 13%, no more than 14%, no more than 15%, no more than 16%, no more than 17%, no more than 18%, no more than 19%, no more than 20%, no more than 21%, no more than 22%, no more than 23%, no more than 24%, no more than 25%, no more than 26%, no more than 27%, no more than 28%, no more than 29%, no more than 30%, no more than 31%, no more than 32%, no more than 33%, no more than 34%, no more than 35%, no more than 36%, no more than 37%, no more than 38%, no more than 39%, no more than 40%, no more than 41%, no more than 42%, no more than 43%, no more than 44%, no more than 45%, no more than 46%, no more than 47%, no more than 48%, no more than 49%, or no more than 50% of the isolated DNA molecules. In another embodiment, the baculoviral DNA are less than 1%, less than 2%, less than 3%, less than 4%, less than 5%, less than 6%, less than 7%, less than 8%, less than 9%, less than 10%, less than 11%, less than 12%, less than 13%, less than 14%, less than 15%, less than 16%, less than 17%, less than 18%, less than 19%, less than 20%, less than 21%, less than 22%, less than 23%, less than 24%, less than 25%, less than 26%, less than 27%, less than 28%, less than 29%, less than 30%, less than 31%, less than 32%, less than 33%, less than 34%, less than 35%, less than 36%, less than 37%, less than 38%, less than 39%, less than 40%, less than 41%, less than 42%, less than 43%, less than 44%, less than 45%, less than 46%, less than 47%, less than 48%, less than 49%, or less than 50% of the isolated DNA molecules. In yet another embodiment, the baculoviral DNA are about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% of the isolated DNA molecules.

The various embodiments the isolated DNA molecules provided herein of various purities with respect to the specific contaminants as described in the preceding paragraphs (e.g. fragments of the DNA molecules, nucleic acid contaminants that are not fragments of the DNA molecules, and/or baculoviral DNA) of this Section 5.4.8 are not mutually exclusive and thus can be combined in various combinations by selecting and combining any embodiments provided in the list of the preceding paragraphs of this Section 5.4.8. Furthermore, the isolated DNA molecules provided in this Section 5.4.8 and those in the paragraphs between the heading of Section 5.4 and the heading of Section 5.4.1 can also be combined in various combinations by selecting and combining any suitable embodiments provided in the list described therein.

### 5.4.9 DNA Molecules Packaged in Viral Particles

The DNA molecules provided herein, including those in Sections 3, 5.2, 5.4, 5.5, and 6, can be packaged in viral particles. Such viral particle can be packaged by transfecting the DNA molecules into a suitable host cell (e.g. HEK 293) and co-transfecting the host cells with other molecules necessary for viral packaging (e.g. viral capsid (Cap) protein), as described in Grieger J C, et al., Nat Protoc 2006; 1:1412-1428; and Liujiang Song, Hum Gene Ther, 2020 October; 31 (19-20): 1054-1067, both of which are incorporated herein in their entireties by reference. One of the advantages of the methods and DNA molecules provided herein is the purity of the DNA molecules provided herein when produced and packaged in viral particles, because the isolated DNA molecules of various purity as provided in Section 5.4.8 can be transfected into host cells and/or packaged in viral particles, thereby providing DNA molecules having various purity in packaged viral particles. Accordingly, the disclosure provides and a person of ordinary skill in the art would understand that the DNA molecules provided herein including in Sections 3, 5.2, 5.4, 5.5, and 6 can be free of certain general DNA contaminants, free of certain specific DNA contaminants, or both free of certain general DNA contaminants and free of certain specific DNA contaminants, when such DNA molecules are packaged in viral particles.

Accordingly, in one embodiment, the DNA molecules packaged in viral particles are free of fragments of the DNA molecules. In another embodiment, the DNA molecules packaged in viral particles are free of nucleic acid contaminants that are not fragments of the DNA molecules. In a further embodiment, the DNA molecules packaged in viral particles are free of baculoviral DNA. In one embodiment, the DNA molecules packaged in viral particles are free of fragments of the DNA molecules and free of nucleic acid contaminants that are not fragments of the DNA molecules. In another embodiment, the DNA molecules packaged in viral particles are free of fragments of the DNA molecules and free of baculoviral DNA. In a further embodiment, the DNA molecules packaged in viral particles are free of baculoviral DNA and free of nucleic acid contaminants that are not fragments of the DNA molecules. In yet another embodiment, the DNA molecules packaged in viral particles are free of fragments of the DNA molecules, free of baculoviral DNA, and free of nucleic acid contaminants that are not fragments of the DNA molecules.

Specifically, in one embodiment, the fragments of the DNA molecules are no more than 1%, no more than 2%, no more than 3%, no more than 4%, no more than 5%, no more than 6%, no more than 7%, no more than 8%, no more than 9%, no more than 10%, no more than 11%, no more than 12%, no more than 13%, no more than 14%, no more than 15%, no more than 16%, no more than 17%, no more than 18%, no more than 19%, no more than 20%, no more than 21%, no more than 22%, no more than 23%, no more than 24%, no more than 25%, no more than 26%, no more than 27%, no more than 28%, no more than 29%, no more than 30%, no more than 31%, no more than 32%, no more than 33%, no more than 34%, no more than 35%, no more than 36%, no more than 37%, no more than 38%, no more than 39%, no more than 40%, no more than 41%, no more than 42%, no more than 43%, no more than 44%, no more than 45%, no more than 46%, no more than 47%, no more than 48%, no more than 49%, or no more than 50% of the DNA molecules packaged in viral particles. In another embodiment, the fragments of the DNA molecules are less than 1%, less than 2%, less than 3%, less than 4%, less than 5%, less than 6%, less than 7%, less than 8%, less than 9%, less than 10%, less than 11%, less than 12%, less than 13%, less than 14%, less than 15%, less than 16%, less than 17%, less than 18%, less than 19%, less than 20%, less than 21%, less than 22%, less than 23%, less than 24%, less than 25%, less than 26%, less than 27%, less than 28%, less than 29%, less than 30%, less than 31%, less than 32%, less than 33%, less than 34%, less than 35%, less than 36%, less than 37%, less than 38%, less than 39%, less than 40%, less than 41%, less than 42%, less than 43%, less than 44%, less than 45%, less than 46%, less than 47%, less than 48%, less than 49%, or less than 50% of the DNA molecules packaged in viral particles. In yet another embodiment, the fragments of the DNA molecules are about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% of the DNA molecules packaged in viral particles.

Additionally, in one embodiment, the nucleic acid contaminants that are not fragments of the DNA molecules are no more than 1%, no more than 2%, no more than 3%, no more than 4%, no more than 5%, no more than 6%, no more than 7%, no more than 8%, no more than 9%, no more than 10%, no more than 11%, no more than 12%, no more than 13%, no more than 14%, no more than 15%, no more than 16%, no more than 17%, no more than 18%, no more than 19%, no more than 20%, no more than 21%, no more than 22%, no more than 23%, no more than 24%, no more than 25%, no more than 26%, no more than 27%, no more than 28%, no more than 29%, no more than 30%, no more than 31%, no more than 32%, no more than 33%, no more than 34%, no more than 35%, no more than 36%, no more than 37%, no more than 38%, no more than 39%, no more than 40%, no more than 41%, no more than 42%, no more than 43%, no more than 44%, no more than 45%, no more than 46%, no more than 47%, no more than 48%, no more than 49%, or no more than 50% of the DNA molecules packaged in viral particles. In another embodiment, the nucleic acid contaminants that are not fragments of the DNA molecules are less than 1%, less than 2%, less than 3%, less than 4%, less than 5%, less than 6%, less than 7%, less than 8%, less than 9%, less than 10%, less than 11%, less than 12%, less than 13%, less than 14%, less than 15%, less than 16%, less than 17%, less than 18%, less than 19%, less than 20%, less than 21%, less than 22%, less than 23%, less than 24%, less than 25%, less than 26%, less than 27%, less than 28%, less than 29%, less than 30%, less than 31%, less than 32%, less than 33%, less than 34%, less than 35%, less than 36%, less than 37%, less than 38%, less than 39%, less than 40%, less than 41%, less than 42%, less than 43%, less than 44%, less than 45%, less than 46%, less than 47%, less than 48%, less than 49%, or less than 50% of the DNA molecules packaged in viral particles. In yet another embodiment, the nucleic acid contaminants that are not fragments of the DNA molecules are about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% of the DNA molecules packaged in viral particles.

In addition, in one embodiment, the baculoviral DNA are no more than 1%, no more than 2%, no more than 3%, no more than 4%, no more than 5%, no more than 6%, no more than 7%, no more than 8%, no more than 9%, no more than 10%, no more than 11%, no more than 12%, no more than 13%, no more than 14%, no more than 15%, no more than 16%, no more than 17%, no more than 18%, no more than 19%, no more than 20%, no more than 21%, no more than 22%, no more than 23%, no more than 24%, no more than 25%, no more than 26%, no more than 27%, no more than 28%, no more than 29%, no more than 30%, no more than 31%, no more than 32%, no more than 33%, no more than 34%, no more than 35%, no more than 36%, no more than 37%, no more than 38%, no more than 39%, no more than 40%, no more than 41%, no more than 42%, no more than 43%, no more than 44%, no more than 45%, no more than 46%, no more than 47%, no more than 48%, no more than 49%, or no more than 50% of the DNA molecules packaged in viral particles. In another embodiment, the baculoviral DNA are less than 1%, less than 2%, less than 3%, less than 4%, less than 5%, less than 6%, less than 7%, less than 8%, less than 9%, less than 10%, less than 11%, less than 12%, less than 13%, less than 14%, less than 15%, less than 16%, less than 17%, less than 18%, less than 19%, less than 20%, less than 21%, less than 22%, less than 23%, less than 24%, less than 25%, less than 26%, less than 27%, less than 28%, less than 29%, less than 30%, less than 31%, less than 32%, less than 33%, less than 34%, less than 35%, less than 36%, less than 37%, less than 38%, less than 39%, less than 40%, less than 41%, less than 42%, less than 43%, less than 44%, less than 45%, less than 46%, less than 47%, less than 48%, less than 49%, or less than 50% of the DNA molecules packaged in viral particles. In yet another embodiment, the baculoviral DNA are about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, or about 50% of the DNA molecules packaged in viral particles.

The disclosure provides that the various contaminants described in this Section 5.4.9 can be detected and determined by various methods known and practiced in the field, for example, by Next-generation sequencing (NGS) or by PCR.

The various embodiments the DNA molecules packaged in viral particles provided herein of various purities with respect to the specific contaminants as described in the preceding paragraphs (e.g. fragments of the DNA molecules, nucleic acid contaminants that are not fragments of the DNA molecules, and/or baculoviral DNA) of this Section 5.4.9 are not mutually exclusive and thus can be combined in various combinations by selecting and combining any embodiments provided in the list of the preceding paragraphs of this Section 5.4.8. Furthermore, the isolated DNA molecules provided in this Section 5.4.8 and those in the paragraphs between the heading of Section 5.4 and the heading of Section 5.4.1 can also be combined in various combinations by selecting and combining any suitable embodiments provided in the list described therein.

### 5.5 Hairpin-Ended DNA Molecules

The disclosure provides that the hairpin-ended DNA molecules of this Section (Section 5.5) can be produced by performing the method steps described in Section 5.2 (including Sections 5.3.3, 5.3.4, and 5.3.5) on DNA molecules provided in Section 5.4. As such, the hairpin-ended DNA molecules of this Section (Section 5.5) can (1) comprise the various features of the DNA molecules provided in Sections 3 and 5.4, including IRs or ITRs that can form hairpins as described in Section 5.4.1 and this Section (Section 5.5), specific sequences, origins, and identities of IRs or ITRs as described in Section 5.4.1 and this Section (Section 5.5), expression cassette as described in 5.4.3, restriction sites for nicking endonucleases or restriction enzymes as described in Sections 5.4.2, 5.3.4, and 5.4.7, and the targeting sites for programmable nicking enzymes as described in Section 5.3.4, and/or (2) lacks the RABS and/or TRS sequences as described in Section 5.4.5. Therefore, the disclosure provides that the hairpin-ended DNA molecules of this Section (Section 5.5) can (1) comprise any combination of embodiments of IRs or ITRs that can form hairpins as described in Sections 5.4.1 and this Section (Section 5.5), expression cassette encoding a human FVIII or a catalytically active fragment thereof as described in 5.4.3, restriction sites for nicking endonucleases or restriction enzymes as described in Sections 5.4.2, 5.3.4, and 5.4.7, the targeting sites for programmable nicking enzymes as described in Section 5.3.4, and additional features for the vectors provided in this Section (Section 5.5), and/or (2) lacks the RABS and/or TRS sequences as described in Section 5.4.5.

As is clear from the descriptions, the ITRs or the hairpinned ITRs in the hairpin-ended DNA molecules provided in this Section (Section 5.5) can be formed from the ITRs or IRs provided above in Sections 3 and 5.4.1, for example upon performing the method steps described in Sections 3, 5.3.3, 5.3.4, and 5.3.5. Accordingly, in some embodiments, the two ITRs or the two hairpinned ITRs in the hairpin-ended DNA molecules provided in this Section (Section 5.5) can comprise any embodiments of the IRs or ITRs provided in Sections 3 and 5.4.1 and additional embodiments provided in this Section (Section 5.5), in any combination.

In one aspect, provided herein is a double strand DNA molecule comprising in 5′ to 3′ direction of the top strand: a.) a first hairpinned inverted repeat (e.g. as described in Section 5.4.1 and this Section (Section 5.5)); b.) a nick of the bottom strand (e.g. as described in Sections 5.3.4 and 5.4.2, and this Section (Section 5.5)); c.) an expression cassette encoding a human FVIII or a catalytically active fragment thereof (e.g. as described 5.4.3 and this Section (Section 5.5)); d.) a nick of the bottom strand (e.g. as described in Sections 5.3.4 and 5.4.2, and this Section (Section 5.5)); and e.) a second hairpinned inverted repeat (e.g. as described in Section 5.4.1 and this Section (Section 5.5)).

In another aspect, provided herein is a double strand DNA molecule comprising in 5′ to 3′ direction of the top strand: a.) a first hairpinned inverted repeat (e.g. as described in Section 5.4.1 and this Section (Section 5.5)); b.) a nick of the top strand (e.g. as described in Sections 5.3.4 and 5.4.2, and this Section (Section 5.5)); c.) an expression cassette encoding a human FVIII or a catalytically active fragment thereof (e.g. as described 5.4.3 and this Section (Section 5.5)); d.) a nick of the top strand (e.g. as described in Sections 5.3.4 and 5.4.2, and this Section (Section 5.5)); and e.) a second hairpinned inverted repeat (e.g. as described in Section 5.4.1 and this Section (Section 5.5)).

In yet another aspect, provided herein is a double strand DNA molecule comprising in 5′ to 3′ direction of the top strand: a.) a first hairpinned inverted repeat (e.g. as described in Section 5.4.1 and this Section (Section 5.5)); b.) a nick of the bottom strand (e.g. as described in Sections 5.3.4 and 5.4.2, and this Section (Section 5.5)); c.) an expression cassette encoding a human FVIII or a catalytically active fragment thereof (e.g. as described 5.4.3 and this Section (Section 5.5)); d.) a nick of the top strand (e.g. as described in Sections 5.3.4 and 5.4.2, and this Section (Section 5.5)); and e.) a second hairpinned inverted repeat (e.g. as described in Section 5.4.1 and this Section (Section 5.5)).

In a further aspect, provided herein is a double strand DNA molecule comprising in 5′ to 3′ direction of the top strand: a.) a first hairpinned inverted repeat (e.g. as described in Section 5.4.1 and this Section (Section 5.5)); b.) a nick of the top strand (e.g. as described in Sections 5.3.4 and 5.4.2, and this Section (Section 5.5)); c.) an expression cassette encoding a human FVIII or a catalytically active fragment thereof (e.g. as described 5.4.3 and this Section (Section 5.5)); d.) a nick of the bottom strand (e.g. as described in Sections 5.3.4 and 5.4.2, and this Section (Section 5.5)); and e.) a second hairpinned inverted repeat (e.g. as described in Section 5.4.1 and this Section (Section 5.5)).

The secondary structure is formed based on conformations (e.g. domains) that include base pair stacking, stems, hairpins, bulges, internal loops, and multi-branch loops. A domain-level description of IRs represents the strand and formed complexes in terms of domains rather than specific nucleotide sequences. At the sequence level, each domain is assigned a particular nucleotide sequence or motif, and its complement's sequence is determined by Watson-Crick base pairing. This spans the full range of binding between any pair of complementary nucleotides, including G-T wobble base pairs. The overall set of bound (e.g. base paired) and unbound domains form a unimolecular complex and exhibit various secondary structure. In some embodiments, hairpins can have a base-paired stem and a small loop of unpaired bases. In certain embodiments, the presence of interweaved non-palindromic polynucleotides sections in the polynucleotide sequence can lead to unpaired nucleotides known as bulges. Bulges can have one or more nucleotides and are classified in different types depending on their location: in the top strand (bulge), in both strands (internal loop) or at a junction. The collection of these base pairs constitutes the secondary structure of DNA, which occur in its three-dimensional structure.

A domain-level description for the DNA molecules provided herein are also provided to represent multiple strands and their complexes in terms of domains rather than specific nucleotide sequences. In some embodiments, domains (e.g. sequences motifs) of interacting single stranded DNA strands can exhibit particular secondary structures on a single strand level that can interact with other DNA strands and in some cases take on a hybridized structure when a first strand is bound to a complementary domain on a second strand to form a duplex. Interactions of different DNA strands that generate new complexes or changes in secondary structure can be viewed as “reactions.” Additional unimolecular and bimolecular reactions are also possible at the sequence level. Poor sequence design can lead to sequence-level structures or interactions (e.g. multiple domains of complimentary in the expression cassette) that interfere with the intended reactions of a system comprising one or more DNA molecules provided herein. Undesired interactions can be avoided by design, resulting in reliable and predictable secondary structure formation.

The disclosure provides that the underlying forces leading to the secondary structure of DNA are governed by hydrophobic interactions that underlie thermodynamic laws and the overall conformation may be influenced by physicochemical conditions. An exemplary list of factors determining equilibrium state include the type of solvent, chemical agents crowding, salt concentrations, pH, and temperature. While free energy change parameters and enthalpy change parameters derived from experimental literature allow for a prediction of conformation stability, the overall three-dimensional structures of the hairpin formed from the IR sequences, as usual in statistical mechanics, corresponds to an ensemble of molecular conformations, not just one conformation. Predominant conformations cam transition as the physical or chemical conditions (e.g. salts, pH, or temperature) are permutated.

“Stem domain” or “stem” refers to a self-complementary nucleotide sequence of the overhang strand that will form Watson-Crick base pairs. The stem comprises primarily Watson-Crick base pairs formed between the two antiparallel stretches of DNA pairs and can be a right-handed helix. In one embodiment, the stem comprises the stretch of self-complimentary DNA sequence in a palindromic sequence.

“Primary stem domain” or “primary stem” refers to the part of self-complementary or reverse complement nucleotide sequences of the ITR that is most proximal to the expression cassette or the non-ITR sequences of the DNA molecule. In one embodiment, the primary stem domain is the self-complimentary stretch of a palindromic sequence that forms the termini of the DNA molecules provided herein and is covalently linked to the non-ITR sequences flanked by the ITRs. The primary stem encompasses both the start as well as the end of an IR sequence. In certain embodiments, the primary stems range in length from 1 to 100 or more bp. In certain embodiments, the primary stems of each ITR independently have a length of from about 1 to about 100 nucleotides. In certain embodiments, the primary stems of each ITR independently have a length of more than 100 nucleotides. In yet another embodiment, the primary stem of each ITR independently has a length of at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, or at least 100 nucleotides. In another embodiment, the primary stem of each ITR independently has a length of about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, or about 100 nucleotides. The lengths of primary stem region of each ITR have an effect on the denature/renature kinetics of the ITR and/or the double-stranded DNA molecule. In some specific embodiments, the primary stem region has between at least about 4 and about 25 nucleotides to ensure thermal stability. In other specific embodiments, the primary stem region has between about 4 and about 25 nucleotides to ensure thermal stability. On the other hand, the inverted repeat domains may be of any length sufficient to maintain an approximate three-dimensional structure at physiological conditions.

“Loop” or “loop domain” refers to the region of unpaired nucleotides in an IR or ITR that is not a turning point and not in a stem. In some embodiments, a loop domain is found at the apex of the IR structure. The loop domain can serve as the region in which the local directionality of the DNA strand is reversed to afford the two antiparallel strands of the originating stem. Because of steric repulsion, in certain embodiments, a loop comprises a minimum of two nucleotides to make a turn in a DNA hairpin. In other embodiments, a loop comprises four nucleotides or more. In yet other embodiments, a loop comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides. In some further embodiments, a loop comprises about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 nucleotides. The loop follows a self-complementary sequence of a stem and serves to connect the further nucleotides to the stem domain. In some embodiments, a loop comprises a sequence of oligonucleotides that does not form contiguous duplex structure with other nucleotides in the loop sequence or other elements of the ITR (e.g., the loop remains in flexible, single-stranded form). In one embodiment, the loop sequence that does not form a duplex with other nucleotides in the loop sequence is a series of identical bases (e.g. AAAAAAAA, CCCCCCCC, GGGGGGG or TTTTTTTT). In one embodiment, the loop contains between 2 and 30 nucleotides. In a further embodiment, the loop domain contains between 2 and 15 nucleotides. In yet a further embodiment, the loop comprises a mixture of nucleotides.

As used herein, the term “hairpin” refers to any DNA structure as well as the overall DNA structure, including secondary or tertiary structure, formed from an IR or ITR sequence. As used herein, a “hairpinned” DNA molecule refers to a DNA molecule wherein one or more hairpins has formed in the DNA molecule. In one embodiment, a hairpin comprises a complementary stem and a loop. A hairpin in its simplest form consists of a complementary stem and a loop. A structure encompassing stems and loops are referred to as “stem-loop,” “stem loop,” or “SL.” In another embodiment, a hairpin consists of a complementary stem and a loop. “Branched hairpin” refers to a subset of hairpin that has multiple stem-loops that form branch structures (e.g. as depicted in FIG. 1). An IR or ITR after forming hairpin can be referred to as hairpinned ITR or IR. A “hairpin-ended” DNA molecule refers to a DNA molecule wherein a hairpin has formed at one end of the DNA molecule, or a hairpin has formed at each of the 2 ends of the DNA molecule.

“Turning point” or “apex” refers to the region of unpaired nucleotides at the spatial end of the ITR. The turning point serves as the region in which the global directionality of the DNA strand is reversed to afford the two antiparallel strands of the originating stem. The turning point also marks the point at which the IR or ITR sequence becomes inverted or the reverse compliment.

In some embodiments, the part of ITR following the primary stem domain, can encode a nucleotide sequence, which in contrast to regular double-stranded DNA, can form non-Watson-Crick-based structural elements when folding on itself, including wobbles and mismatches, and structural defects or imperfections, such as bulges and internal loops (see e.g. FIG. 1). A “bulge” contains one or more unpaired nucleotides on one strand, whereas “internal loops” contain one or more unpaired nucleotides on both top and bottom strands. Symmetric internal loops tend to distort the helix less than bulges and asymmetric internal loops, which can kink or bend the helix. In some embodiments, the unpaired nucleotides in a stem can engage in diverse structural interactions, such as noncanonical hydrogen bonding and stacking, which lend themselves to additional thermodynamic stability and functional diversity. Without being bound by theory, it is thought that the structural diversity of IR stems and loops leads to complex secondary structures, and functional diversity.

In some embodiment, a hairpin for the hairpin-ended DNA molecule comprises a primary stem. In one embodiment, a hairpin for the hairpin-ended DNA molecule comprises 1, 2, 3, 4, 5, 6, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 stems. In another embodiment, a hairpin for the hairpin-ended DNA molecule comprises 1, 2, 3, 4, 5, 6, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 loops. In yet another embodiment, a hairpin for the hairpin-ended DNA molecule comprises 1, 2, 3, 4, 5, 6, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 internal loops. In a further embodiment, a hairpin for the hairpin-ended DNA molecule comprises 1, 2, 3, 4, 5, 6, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 bulges. In one embodiment, a hairpin for the hairpin-ended DNA molecule comprises 1, 2, 3, 4, 5, 6, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 branched hairpins. In another embodiment, a hairpin for the hairpin-ended DNA molecule comprises 1, 2, 3, 4, 5, 6, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 apexes. In a further embodiment, a hairpin for the hairpin-ended DNA molecule comprises any number of stems, branched hairpins, loops, bulges, apexes, and/or internal loops, in any combination.

In some embodiments, the hairpin structure in the DNA molecules provided herein is formed by a symmetrical overhang. In order to obtain a symmetrical overhang, the modification in the 5′ stem region will require a cognate 3′ modification at the corresponding position in the stem region so that the modified 5′ position(s) can form base pair(s) with the modified 3′ position(s). Such modification to form a symmetrical overhang can be performed as described in the present disclosure in combination with the state of the art at the time of filing. For example, by generating a BstNBI restriction site for nicking endonuclease by an insertion of an A at position 23 will require an insertion of T at position 105 with respect to the wt AAV2 ITR (e.g., SEQ ID NO:162).

In some embodiments, the 5′ and 3′ hairpinned ITRs from a hairpinned ITR pair can have different reverse complement nucleotide sequences to harbor the antiparallel restriction sites for nicking endonuclease (e.g. 5′ ITR such that nicking results in a bottom strand 5′ overhang and the 3′ ITR such that nicking results in a bottom strand 3′ overhang) but still have the same three-dimensional spatial organization such that both ITRs have mutations that result in the same overall 3D shape.

In some embodiments, hairpinned ITRs for use herein can comprise a modification (e.g., deletion, substitution or addition) of at least 1, 2, 3, 4, 5, 6, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in any one or more of the regions selected from: the primary stem domain, a stem, a branched hairpin, a loop, a bulge or an internal loop. In one specific embodiment, the nucleotide in a right hairpinned ITR can be substituted from an A to a G, C or T or deleted or one or more nucleotides added; a nucleotide in a left hairpinned ITR can be changed from a T to a G, C or A, or deleted or one or more nucleotides added.

In some embodiments, hairpinned ITRs for use herein can comprise a modification (e.g., deletion, substitution or addition) of at least 1, 2, 3, 4, 5, 6, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in any one or more of the regions selected from: the primary stem domain, a stem, a branched hairpin, a loop, a bulge or an internal loop, in order to replace or deplete the occurrence of CpG motifs, thereby; (i) reducing or eliminating the binding of such modified hairpinned ITRs to toll like family of receptors (TLRs) (e.g. TLR9) compared to viral wild type ITRs and/or (ii) reduce or diminish ITR transcriptional activity by removing transcriptionally active CpG islands. Transcriptionally active CpG islands are commonly defined as sequences with a C+G ratio of greater than 50% and observed-to-expected CpG dinucleotides at 60% or higher as described in Gardiner-Garden M, Frommer M. CpG Islands in vertebrate genomes. J Mol Biol 1987; 196:261-282. In one specific embodiment, the nucleotide in a right hairpinned ITR can be substituted from an G or C to a A or T or deleted or one or more nucleotides added between a C and G or a G and C; a nucleotide in a left hairpinned ITR can be changed from a C or G to a T or A, or deleted or one or more nucleotides added between a C and G or a G and C. In one specific embodiment the hairpinned ITRs comprise a CpG depleted sequence of

(SEQ ID NO: 336)
TTGGTCACTCCCTCTCTGTACACTCACT
CACTCACTGATCCCTGGATACCAAAGGT
ATCCAGACACCCAGTCTTTGACTGGGTG
GGATCAGTGAGTGAGTGAGTGTACAGAG
AGGGAGTGACCAA

In some embodiments, hairpinned ITRs for use herein can comprise a modification (e.g., deletion, substitution or addition) of at least 1, 2, 3, 4, 5, 6, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in any one or more of the regions selected from: the primary stem domain, a stem, a branched hairpin, a loop, a bulge or an internal loop, to comprise one or more telomeric sequences (e.g., TTAGGG, TCAGGG or TGAGGG), thereby; (i) reducing or eliminating the activation of such modified hairpinned ITRs to toll like family of receptors (TLRs) (e.g. TLR9), (ii) reduce or diminish ITR transcriptional activity by removing transcriptionally active CpG islands, and/or (iii)) reduce or diminish cGAS activation, compared to viral wildtype ITRs. In some embodiments, hairpinned ITRs for use herein can comprise a modification (e.g., deletion, substitution or addition) resulting a hairpinned ITR comprising at least one telomeric sequence repeat, at least two telomeric sequence repeats, at least three telomeric sequence repeats, at least four telomeric sequence repeats or at least five telomeric sequence repeats. In some embodiments, hairpinned ITRs for use herein can comprise a modification (e.g., deletion, substitution or addition) of the primary stem domain resulting in a primary stem domain comprising a sequence of TBAGGGTBAGGGTBAGGGTBAGGG, where B is a T, G or C. In specific embodiments, hairpinned ITRs for use herein can comprise a modification (e.g., deletion, substitution or addition) of the primary stem domain, resulting in a primary stem domain comprising a sequence of TBAGGGTTAGGGTTAGGGTTAGGG, where B is a T, G or C. In one specific embodiment the hairpinned ITR of the DNA molecules provided herein comprise a telomeric sequence repeat (e.g. TTTGAGGGTTAGGGTTAGGGTTAGGGTCCCTTTAGGGCAAAGCCCTAAAGACAC CCTAACTTTGTTAGGGTGGGACCCTAACCCTAACCCTAACCCTCAAA (SEQ ID NO 339 or as shown in FIG. 29). In certain embodiments, at least one hairpinned ITR of the DNA molecules provided herein comprises a nucleotide sequence at least about 60%, at least about 70%, at least about 80%, at least about 90%, at least about 95%, at least about 96%, at least about 97%, at least about 98%, at least about 99%, or about 100% identical to SEQ ID NO: 339.

In some embodiments, the hairpinned ITR of the DNA molecules provided herein may exhibit reduce or eliminate innate immune activation and thereby enhance efficacy of the DNA molecules by increasing and/or prolonging FVIII expression levels due to the reduced immune-activated state of the host cell, as compared to DNA molecules comprising wildtype viral sequences.

In some embodiments, the hairpinned ITR of the DNA molecules provided herein can comprise primary stem wherein 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more complementary base pairs are removed from each of the primary stem domains such that the primary stem domain is shorter and has a lower free energy of folding. Briefly, in such embodiments, if a base is removed in the portion of the primary stem domain, the complementary base pair in primary stem domain is also removed, thereby shortening the overall primary stem domain.

In some embodiments, the hairpinned ITR of the DNA molecules provided herein can comprise primary stem wherein 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more complementary base pairs are introduced from each of the primary stem domains such that the primary stem domain is longer and has a higher free energy of folding. Briefly, in such embodiments, if a base is introduced in the portion of the primary stem domain, the complementary base pair in primary stem domain is also introduced, thereby lengthening the overall primary stem domain.

In some embodiments, the hairpinned ITR of the DNA molecules provided herein can comprise primary stem wherein 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more complementary base pairs are substituted from A or T to G or C from each of the primary stem domains such that the primary stem domain is more G/C rich and has a higher free energy of folding. Briefly, in such embodiments, if a base is substitute (e.g. T to G) in the portion of the primary stem domain, the complementary base pair in primary stem domain is also substituted (e.g. A to C, thereby increasing the G/C content the overall primary stem domain.

In further embodiments, the hairpinned ITR of the DNA molecules provided herein can comprise a primary stem wherein 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more complementary base pairs are substituted from G or C to A or T, or deleted or one or more nucleotides added between a C and G or a G and C; from each of the primary stem domains such that the primary stem domain contains less or no CpG motifs and has a lower TLR9 binding propensity than a viral ITR and/or fewer transcriptionally active CpG islands compared to a reference DNA (e.g. the same DNA molecule but with a unmodified primary stem sequence comprising CpG motifs).

In yet another embodiment, the hairpinned ITR of the DNA molecules provided herein can comprise a primary stem wherein 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more complementary base pairs are substituted from G or C to A or T from each of the primary stem domains such that RAPs (e.g. Rep) can no longer efficiently bind to the primary stem domain.

In yet another embodiment, the hairpinned ITR of the DNA molecules provided herein can comprise a primary stem wherein 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more complementary base pairs are substituted from A or T to G or C from each of the primary stem domains such that the primary stem domain is more G/C rich and has a higher free energy of folding such that RAPs (e.g. Rep or NS1) can no longer efficiently bind to the primary stem domain.

In some embodiments, a hairpinned ITR sequence in the DNA molecules provide herein can have between 1 and 40 nucleotide deletions relative to a full-length WT viral ITR sequence while the whole wt ITR sequence is still present in the vector. For example, in a symmetric ITR such as the AAV2 ITR, if restriction sites for nicking endonuclease are each 25 bases away from the Apex, the portion after the restriction site for nicking endonuclease of the overhang does not need to be the wt IR sequence as it will be removed from the DNA molecules after incubation with nicking endonuclease (or nicking endonuclease and restriction enzymes) and denaturing as described in Sections 5.3.3 and 5.3.4. In certain embodiments, a hairpinned ITR sequence in the DNA molecules provide herein can have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotide deletions relative to a full-length WT viral ITR sequence while the whole wt ITR sequence is still present in the vector.

In some embodiments, the restriction site for nicking endonuclease is chosen based on the predicted melting temperature of the isolated nucleotide sequence present in the ITR stem region. In some embodiments, the predicted melting temperature is between 40-95° C. Further embodiments are for the restriction site for nicking endonuclease and the embodiments factoring in melting temperature are described in Sections 5.3.3, 5.3.4, 5.3.5 and 5.4.2 above.

In one embodiment, the length and GC content of the nucleotide sequence encompassing stem region of a hairpinned ITR in a DNA molecule provided herein is further modified by a deletion, insertion, and/or substitution so that a hairpin forms when the temperature is maintained at approximately 4° C. For example, the nucleotide sequence of the structural element can be modified as compared to the wild-type sequence of a viral ITR. In one embodiment, the length and GC content of the stem is designed so that a hairpin forms when the temperature is maintained at approximately 10° C. or more below the melting temperature of the total ITR. The hairpin's melting temperature can be designed by changing the GC content, distance between restriction sites for nicking endonuclease and the junction closest to the primary stem (e.g. number 4 in FIG. 1), or sequence mismatch or loop, so that the melting temperature is high enough to allow the hairpinned ITR to remain folded above 50° C. to ensure stable storage. The actual optimal length of the stem can vary with sequence of ITR and micro domains such as branches, loops, and arms of the ITR, which can be determined according to the present disclosure in combination of the state of the art.

In some embodiments, the stem region of the hairpinned ITR encode a restriction site for Class II nicking endonuclease (e.g. NNNN downstream of 5′). In some embodiments, the stem region does not contain a restriction site for Class II nicking endonuclease.

In some embodiments, the stem region of the hairpinned ITR encode a restriction site for Class I nicking endonuclease. In some embodiments, the stem region of the hairpinned ITR encode a restriction site for Class III, IV or V nicking endonuclease. FIG. 3 depicts various exemplary arrangements of the restriction sites for endo nuclease in the primary stem of a hairpin.

In some embodiments, the expression cassette in the hairpin-ended DNA molecules can be any embodiments of the expression cassette described in Section 5.4.3. In certain embodiments, the ITRs in the hairpin-ended DNA molecules can be any embodiments of the IR or ITR described in Section 5.4.1. In further embodiments, the arrangement among the ITR, the expression cassette, and the restriction sites for nicking endonuclease or restriction enzymes can be any arrangement as described in Sections 5.3.3, 5.3.4, 5.3.5, 5.4.1, 5.4.2, 5.4.3 and 5.4.7.

In some embodiments, the hairpin-ended DNA comprises a top strand that is covalently linked to the 3′ ITR as well as 5′ ITR and once the ITR is folded, the bottom strand is flanked by two nicks (a first and a second nick) at either end of the bottom strand such that the expression cassette is in between the first nick and the second nick, wherein the first nick is formed between the 3′ end of the bottom strand and the juxtaposed 5′ end of the top strand as a result of top strand 5′ ITR hairpin and the second nick is formed between the 5′ end of the bottom strand and the juxtaposed 3′ end of the top strand as a result of top strand 3′ ITR hairpin.

In some embodiments, the hairpin-ended DNA comprises a bottom strand that is covalently linked to the 3′ ITR as well as 5′ ITR and once the ITR is folded, the top strand is flanked by two nicks (a first nick and a second nick) at either end of the top strand such that the expression cassette is in between the first nick and the second nick, wherein the first nick is formed between the 5′ end of the top strand and the juxtaposed 3′ end of the bottom strand as a result of bottom strand 3′ ITR hairpin and the second nick is formed between the 3′ end of the top strand and the juxtaposed 5′ end of the bottom strand as a result of bottom strand 3′ ITR hairpin.

In some embodiments, the hairpin-ended DNA comprises a top strand that is covalently linked to the 5′ ITR and the bottom strand is covalently linked to the 5′ ITR so that when the ITRs are folded, the first nick is formed adjacent to the bottom strand between the 3′ end of the bottom strand and the juxtaposed 5′ end of the top strand as a result of top strand 5′ ITR hairpin and the second nick is formed adjacent to the top strand between the 3′ end of the top strand and the juxtaposed 5′ end of the bottom strand as a result of bottom strand 5′ ITR hairpin, with the expression cassette being flanked by the first and second nicks.

In some embodiments, the hairpin-ended DNA comprises a top strand that is covalently linked to the 3′ ITR and the bottom strand is covalently linked to the 3′ ITR so that when the ITRs are folded, the first nick is formed adjacent to the top strand between the 5′ end of the top strand and the juxtaposed 3′ end of the bottom strand as a result of bottom strand 3′ ITR hairpin and the second nick is formed adjacent to the bottom strand between the 5′ end of the bottom strand and the juxtaposed 3′ end of the top strand as a result of top strand 3′ ITR hairpin, with the expression cassette being flanked by the first and second nicks.

In some embodiments, the hairpin-ended DNA comprising the two nicks as described in this Section (Section 5.5) and the preceding 4 paragraphs can be ligated to repair the nicks by forming a covalent bond between the two nucleotides flanking the nick. In some embodiments, one of the two nicks described in this Section (Section 5.5) and the preceding 4 paragraphs can be ligated and repaired such that when denatured, the DNA molecule becomes a linear single stranded DNA molecule. In some embodiments, the two nicks described in this Section (Section 5.5) and the preceding 4 paragraphs can be ligated and repaired such that when denatured, the DNA molecule becomes a circular single stranded DNA molecule.

In some embodiments, the two flanking ITR pairs in the hairpin-ended DNA molecule comprise identical DNA sequence. In some embodiments, the two flanking ITR pairs in the hairpin-ended DNA molecule comprise different DNA sequences. In some embodiments, one of the ITRs in the hairpin-ended DNA molecule is modified by deletion, insertion, and/or substitution as compared to the other ITR in the same hairpin-ended DNA molecule. In another embodiment, the first ITR and the second ITR in the hairpin-ended DNA molecule are both modified, e.g. by deletion, insertion, and/or substitution. In yet another embodiment, the first ITR and the second ITR in the hairpin-ended DNA molecule comprise different DNA sequences and are both modified. In a further embodiment, the first ITR and the second ITR in the hairpin-ended DNA molecule comprise different DNA sequences and are both modified, wherein the modifications for the two ITRs are different. In yet a further embodiment, the first ITR and the second ITR in the hairpin-ended DNA molecule comprise different DNA sequences and are both modified, wherein the modifications for the two ITRs are identical. In one embodiment, the first ITR and the second ITR in the hairpin-ended DNA molecule comprise identical DNA sequence and are both modified, wherein the modifications for the two ITRs are different. In one embodiment, the first ITR and the second ITR in the hairpin-ended DNA molecule comprise identical DNA sequence and are both modified, wherein the modifications for the two ITRs are identical. In one embodiment, the first ITR and the second ITR in the hairpin-ended DNA are both modified ITRs and the two modified ITRs are not identical. In some embodiments, the hairpin-ended DNA molecules comprise two ITRs that are asymmetric, wherein the asymmetry can be a result of any changes in one ITR that are not reflected in the other ITR. In certain embodiments, the hairpin-ended DNA molecules comprise two ITRs that are asymmetric, wherein the ITRs are different with respect to each other in any way. In certain embodiments, the modifications provided in this paragraph, including deletion, insertion, and/or substitution, can be any such modifications described above in this Section (Section 5.5).

In one aspect, a hairpin-ended DNA molecule provided herein comprises, in the 5′ to 3′ direction: a first IR, a nucleotide sequence of interest and a second IR. In one embodiment, the nucleotide sequence of interest comprises an expression cassette encoding a human FVIII or a catalytically active fragment thereof as described herein, e.g. in Sections 5.4.3. In certain embodiments, the hairpin-ended DNA molecules provided herein including in Section 3 and this Section (Section 5.5) comprise an expression cassette encoding a therapeutic FVIII polypeptide, wherein the expression cassette can be any embodiments described in Sections 3 and 5.4.3.

In some embodiments, the hairpin-ended DNA molecules provided herein including in Section 3 and this Section (Section 5.5), comprise in the 5′ to 3′ direction: a first IR, an expression cassette encoding a human FVIII or a catalytically active fragment thereof as described in 5.4.3 and a second IR, wherein said hairpin-ended DNA molecule comprises 0, at most 1, at most 2, at most 3, at most 4, at most 5, at most 6, at most 8, at most 9, at most 10, at most 15, at most 20 or at most 30 CpG motifs. In a specific embodiment, the hairpin-ended DNA molecules provided herein including in Section 3 and this Section (Section 5.5), comprise in the 5′ to 3′ direction: a first IR, an expression cassette encoding a human FVIII or a catalytically active fragment thereof as described in 5.4.3 and a second IR, wherein said hairpin-ended DNA molecule comprises no CpG motifs. In yet some other embodiments, hairpin-ended DNA molecules provided herein including this Section (Section 5.5), comprise in the 5′ to 3′ direction: a first IR, an expression cassette encoding a human FVIII or a catalytically active fragment thereof comprising a promoter operatively linked to a transcription unit as described in 5.4.3, and a second IR, wherein said hairpin-ended DNA molecule comprises no CpG motifs other than at most 1, at most 2, at most 3, at most 4, at most 5, at most 6, at most 8, at most 9, at most 10, at most 15, at most 20 or at most 30 CpG motifs in the promoter sequence of the expression cassette.

The hairpin-ended DNA molecules can comprise a combination of dsDNA and ssDNA. In some embodiments, certain portion of the hairpin-ended DNA molecules provided in this Section (Section 5.5) is dsDNA. In some embodiments, the dsDNA portion of the hairpin-ended DNA molecules provided in this Section (Section 5.5) comprises the expression cassette, a stem region of the ITR, or both. In some embodiments, certain portion of the hairpin-ended DNA molecules provided in this Section (Section 5.5) is ssDNA. In further embodiments, the dsDNA portion of the hairpin-ended DNA molecules provided in this Section (Section 5.5) comprises the expression cassette, a stem region of the ITR, or both. In one embodiment, the dsDNA portion of the hairpin-ended DNA molecules provided in this Section (Section 5.5) accounts for over 90% of the hairpin-ended DNA molecules. In another embodiment, the dsDNA portion of the hairpin-ended DNA molecules provided in this Section (Section 5.5) accounts for at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% of the hairpin-ended DNA molecules. In another embodiment, the dsDNA portion of the hairpin-ended DNA molecules provided in this Section (Section 5.5) accounts for about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% of the hairpin-ended DNA molecules.

In some embodiments, the hairpin-ended DNA molecule provided herein can be efficiently targeted or transported to the nucleus of a cell. In one embodiment, the hairpin-ended DNA molecule provided herein can be efficiently targeted or transported to the nucleus of a cell by the binding between the aptamer formed at the ITR and a nucleus protein. In another embodiment, the hairpin-ended DNA molecule provided herein can be efficiently targeted or transported to the nucleus of a cell, such that the abundance of the hairpin-ended DNA molecules in the nucleus is 10%, 20%, 30%, 40%, 50%, 60%, 70%, 80%, 90%, or 100% higher than that in the cytoplasm. In yet another embodiment, the hairpin-ended DNA molecule provided herein can be efficiently targeted or transported to the nucleus of a cell, such that the abundance of the hairpin-ended DNA molecules in the nucleus is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 fold higher than that in the cytoplasm.

In various embodiments of the hairpin-ended DNA molecule provided herein including in Section (Section 5.5), the hairpin-ended DNA molecule lacks the RABS and/or TRS sequences as described in Section 5.4.5. In others embodiments of the hairpin-ended DNA molecule provided herein including in Section (Section 5.5), the hairpin-ended DNA molecule lacks any or any combination of the DNA sequences, elements, or features as described in Section 5.4.5.

In some additional embodiments, embodiments of the hairpin-ended DNA molecule provided herein including in Section (Section 5.5), the hairpin-ended DNA molecule can be an isolated hairpin-ended DNA molecules in any embodiment with respect to purity as described in Section 5.4.8.

### 5.6 Functional Properties of the Hairpin-Ended DNA Molecules

In some embodiments, the ITR promotes the long-term survival of the nucleic acid molecule in the nucleus of a cell. In some embodiments, the ITR promotes the permanent survival of the nucleic acid molecule in the nucleus of a cell (e.g., for the entire lifespan of the cell). In some embodiments, the ITR promotes the stability of the nucleic acid molecule in the nucleus of a cell. In some embodiments, the ITR inhibits or prevents the degradation of the nucleic acid molecule in the nucleus of a cell.

In some embodiments, when the ITR assumes its folded state, it is resistant to exonuclease digestion (e.g. exonuclease V), e.g. for over an hour at 37° C. In one embodiment, the hairpin-ended DNA molecule is resistant to exonuclease digestion (e.g. digestion by exonuclease V). In another embodiment, the hairpin-ended DNA molecule is resistant to exonuclease digestion (e.g. digestion by exonuclease V) for at least 1, at least 2, at least 3, at least 4, at least 5, at least 6, at least 7, at least 8, at least 9, at least 10 or more hours. In yet another embodiment, the hairpin-ended DNA molecule is resistant to exonuclease digestion (e.g. digestion by exonuclease V) for about 1, about 2, about 3, about 4, about 5, about 6, about 7, about 8, about 9, or about 10 hours. In some embodiments, hairpin-ended DNA molecule that are resistant to exonuclease digestion (e.g. exonuclease V), e.g. for over an hour at 37° C., are transcriptionally active (e.g. express FVIII) for longer

As unexpectedly found by the discloses and provided herein, linear DNA vectors with ITRs similar to viral ITRs can be produced without the need for RAPs and consequently independent of the RABS or TRS sequence for DNA replication. Accordingly, the RABS and TRS can optionally be encoded in the nucleotide sequence disclosed herein but are not required and offer flexibility with regard to designing the ITRs. One of the advantages of the DNA molecules encoding a human FVIII or a catalytically active fragment provided herein and described in Section 5.5, may be that the flexibility with regard to designing the ITRs (e.g. modified ITRs as described in Section 5.4.1) can confer additional functionalities to the DNA molecules without being bound to any or all limitations of ITRs comprising viral sequences (e.g. RABS or TRS). In some embodiments, said additional functionalities of the DNA molecules provided may include (i) block residual transcriptional activity as modified ITR sequences lacking RABS or TRS may have no or diminished endogenous promoter and/or transcriptional activity (e.g. the P5 AAV promoter, which shares a homolog sequence with the RBS) once in a host cell when compared with DNA molecules comprising transcriptionally active CpG islands of wildtype viral RABS or TRS sequences, (ii) circumvent TLR9 recognition by reducing or eliminating the binding of such modified ITRs lacking RABS or TRS sequences to toll like family of receptors (TLRs) (e.g. TLR9) once in a host cell when compared with DNA molecules comprising CpG motifs present in wildtype viral RABS or TRS sequences, (iii) hamper innate immune recognition by antagonist binding of such modified ITRs comprising telomeric repeat sequences (as described in Section 5.5) and lacking RABS or TRS sequences, to cellular double-stranded DNA sensors (e.g. TLR9, cyclic GMP-AMP synthase, etc.) compared with DNA molecules comprising sequences (such as CpG motifs) present in wildtype viral RABS or TRS sequences and/or impede the risk of mobilization or replication once administered to a patient when compared with DNA molecules including such RABS and/or TRS sequences. In some embodiments, said additional functionalities of the DNA molecules lacking wildtype viral RABS or TRS sequences provided herein may reduce or eliminate such immune responses and thereby enhance efficacy of the DNA molecules by increasing and/or prolonging FVIII expression levels due to the reduced immune-activated state of the host cell as compared to DNA molecules comprising wildtype viral RABS or TRS sequences. In one embodiment, the DNA molecules provided herein comprise ITRs that do not comprise RABS. In another embodiment, the DNA molecules provided herein comprise ITRs that do not comprise TRS. In yet another embodiment, the DNA molecules provided herein comprise ITRs that do not comprise either RABS or TRS. In a further embodiment, the DNA molecules provided herein comprise ITRs that comprise RABS, TRS, or both RABS and TRS.

In some embodiments, the hairpin-ended DNA molecules provided herein are stable in the host cell. In some embodiments, the hairpin-ended DNA molecules provided herein are stable in the host cell for long term culture.

In certain embodiments, the hairpin-ended DNA molecules provided herein can be efficiently delivered to a host cell.

The DNA molecules provided herein have superior stability not just for their resistance to exonuclease digestion described above, but also with respect to their structure. In one embodiment, the structure of the DNA molecules remains the same after storage at room temperature for 1 days, 2 days, 3 days, 4 days, 5 days, 6 days, 7 days, 2 weeks, 3 weeks, 4 weeks, 5 weeks, 6 weeks, 7 weeks, 8 weeks, 3 months, 4 months, 5 months, 6 months, 7 months, 8 months, 9 months, 10 months, 11 months, or 12 months. In another embodiment, the ensemble structure of the DNA molecules remains the same after storage at room temperature for 1 days, 2 days, 3 days, 4 days, 5 days, 6 days, 7 days, 2 weeks, 3 weeks, 4 weeks, 5 weeks, 6 weeks, 7 weeks, 8 weeks, 3 months, 4 months, 5 months, 6 months, 7 months, 8 months, 9 months, 10 months, 11 months, or 12 months. In some embodiments, the structure of the DNA molecules provided herein is the same after 2, 3, 4, 5, 10 or 20 cycles of denaturing/renaturing (e.g. denaturing as described in Section 5.3.3 and re-annealing as described in Section 5.3.5) . . . . DNA structures can be described by an ensemble of structures at or around the energy minimum. In certain embodiments, the ensemble DNA structure is the same after 2, 3, 4, 5, 10 or 20 cycles of denaturing/renaturing. In one embodiment, the folded hairpin structure formed from the ITR or IR provided herein is the same after 2, 3, 4, 5, 10 or 20 cycles of denaturing/renaturing. In another embodiment, the ensemble structure of the folded hairpin is the same after 2, 3, 4, 5, 10 or 20 cycles of denaturing/renaturing.

### 5.7 Delivery Vehicles Comprising the Hairpin-Ended DNA Molecules

In some embodiments, the hairpin-ended DNA molecules provided herein can be delivered via a hybridosome as described in U.S. Pat. No. 10,561,610, which is herein incorporated in its entirety by reference. In other embodiments, the DNA molecules provided herein can be delivered via a hybridosome.

In certain embodiments, the DNA molecules provided herein can be delivered via lipid particles including lipid nanoparticles. In other embodiments, the hairpin-ended DNA molecules provided herein can be delivered via lipid nanoparticles. In some embodiments, the lipid nanoparticle comprises any one or more lipids selected from ionizable lipid, non-cationic lipid (e.g. phospholipid), a sterol (e.g., cholesterol) and a PEGylated lipid. In one embodiment, the lipid particle comprises any one or more lipids selected from ionizable lipid, non-cationic lipid (e.g. phospholipid), a sterol (e.g., cholesterol) and a PEGylated lipid, where the molar ratio of lipids ranges from 20 to 70 mole percent or 40 to 60 mole percent for the ionizable lipid, the mole percent of non-cationic lipid ranges from 0 to 30 or 0 to 15, the mole percent of sterol ranges from 20 to 70 or 30 to 50, and the mole percent of PEGylated lipid ranges from 1 to 6 or 2 to 5. In another embodiment, the lipid particle comprises any one or more lipids selected from ionizable lipid, non-cationic lipid (e.g. phospholipid), a sterol (e.g., cholesterol) and a PEGylated lipid, where the molar ratio of lipids ranges from 40 to 60 mole percent for the ionizable lipid, the mole percent of non-cationic lipid ranges from 0 to 15, the mole percent of sterol ranges from 30 to 50, and the mole percent of PEGylated lipid ranges from 2 to 5. In yet another embodiment, the lipid particle comprises any one or more lipids selected from ionizable lipid, non-cationic lipid (e.g. phospholipid), a sterol (e.g., cholesterol) and a PEGylated lipid, where the molar ratio of lipids ranges from 20 to 70 mole percent for the ionizable lipid, the mole percent of non-cationic lipid ranges from 0 to 30, the mole percent of sterol ranges from 20 to 70, and the mole percent of PEGylated lipid ranges from 1 to 6.

The disclosure provides that ionizable lipids can be used employed to condense the nucleic acid cargo, at low pH and to drive membrane association and fusogenicity. Such ionizable lipids can be used as part of the delivery vehicle for the compositions of and methods for the DNA molecules provided herein. In some embodiments, ionizable lipids are lipids comprising at least one amino group that is positively charged or becomes protonated under acidic conditions, for example at pH of 6.5 or lower. In some embodiments, ionizable lipids have at least one protonatable or deprotonatable group, such that the lipid is positively charged at a pH at or below physiological pH (e.g., pH 7.4), and neutral at a second pH, for example at or above physiological pH. It will be understood by one of ordinary skill in the art that the addition or removal of protons as a function of pH is an equilibrium process, and that the reference to a charged or a neutral lipid refers to the nature of the predominant species and does not require that all of the lipid be present in the charged or neutral form. Generally, ionizable lipids have a pKa of the protonatable group in the range of about 4 to about 7.

Further exemplary ionizable lipids are described in PCT patent publications WO2015/095340, WO2015/199952, WO2018/011633, WO2017/049245, WO2015/061467, WO2012/040184, WO2012/000104, WO2015/074085, WO2016/081029, WO2017/004143, WO2017/075531, WO2017/117528, WO2011/022460, WO2013/148541, WO2013/116126, WO2011/153120, WO2012/044638, WO2012/054365, WO2011/090965, WO2013/016058, WO2012/162210, WO2008/042973, WO2010/129709, WO2010/144740, WO2012/099755, WO2013/049328, WO2013/086322, WO2013/086373, WO2011/071860, WO2009/132131, WO2010/048536, WO2010/088537, WO2010/054401, WO2010/054406, WO2010/054405, WO2010/054384, WO2012/016184, WO2009/086558, WO2010/042877, WO2011/000106, WO2011/000107, WO2005/120152, WO2011/141705, WO2013/126803, WO2006/007712, WO2011/038160, WO2005/121348, WO2011/066651, WO2009/127060, WO2011/141704, WO2006/069782, WO2012/031043, WO2013/006825, WO2013/033563, WO2013/089151, WO2017/099823, WO2015/095346, and WO2013/086354, all of which are herein incorporated in their entirety by reference.

In some specific embodiments, the ionizable lipid is MC3 (6Z,9Z,28Z,31Z)-heptatriaconta-6,9,28,31-tetraen-19-yl-4-(dimethylamino) butanoate (DLin-MC3-DMA or MC3).

In some specific embodiments, the ionizable lipid is MC3 (6Z,9Z,28Z,31Z)-heptatriaconta-6,9,28,31-tetraen-19-yl-4-(dimethylamino) butanoate (DLin-MC3-DMA or MC3).

In some specific embodiments, the ionizable lipid is (((4-hydroxybutyl)azanediyl)bis(hexane-6,1-diyl)bis(2-hexyldecanoate).

In some specific embodiments, the ionizable lipid is 9-Heptadecanyl 8-{(2-hydroxyethyl)[6-oxo-6-(undecyloxy) hexyl]amino}octanoate.

In some embodiments, the lipid nanoparticles encapsulation the DNA molecule of provided herein include one or more lipids selected from the group consisting of distearoyl-phosphatidylcholine (DSPC), dioleoyl-phosphatidylcholine (DOPC), dipalmitoyl-phosphatidylcholine (DPPC), dioleoyl-phosphatidylglycerol (DOPG), dipalmitoyl-phosphatidylglycerol (DPPG), dioleoyl-phosphatidylethanolamine (DOPE), palmitoyloleoyl-phosphatidylcholine (POPC), palmitoyloleoyl-phosphatidylethanolamine (POPE) and dioleoyl-phosphatidy-lethanolamine, dipalmitoyl-phosphatidyl-ethanolamine (DPPE), dimyristoylphospho-ethanolamine (DMPE), distearoyl-phosphatidyl-ethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE).

Delivery vehicles provided herein include those for delivering the DNA molecules provided herein to cells, which sometime are referred to as transfection. Further useful transfection methods include, but are not limited to, lipid-mediated transfection, cationic polymer-mediated transfection, or calcium phosphate precipitation. Transfection reagents well known in the art are provided herein and include, but are not limited to, TurboFect Transfection Reagent (Thermo Fisher Scientific), Pro-Ject Reagent (Thermo Fisher Scientific), TRANSPASS™ P Protein Transfection Reagent (New England Biolabs), CHARIOT™ Protein Delivery Reagent (Active Motif), PROTEOJUICE™ Protein Transfection Reagent (EMD Millipore), 293fectin, LIPOFECT AMINE™ 2000, LIPOFECT AMINE™ 3000 (Thermo Fisher Scientific), LIPOFECT AMINE™ (Thermo Fisher Scientific), LIPOFECTIN™ (Thermo Fisher Scientific), DMRIE-C, CELLFECTIN™ (Thermo Fisher Scientific), OLIGOFECT AMINE™ (Thermo Fisher Scientific), LIPOFECT ACE™, FUGENE™ (Roche, Basel, Switzerland), FUGENE™ HD (Roche), TRANSFECT AM™ (Transfectam, Promega, Madison, Wis.), TFX-10™ (Promega), TFX-20™ (Promega), TFX-50™ (Promega), TRANSFECTIN™ (BioRad, Hercules, Calif.), SILENTFECT™ (Bio-Rad), Effectene™ (Qiagen, Valencia, Calif.), DC-chol (Avanti Polar Lipids), GENEPORTER™ (Gene Therapy Systems, San Diego, Calif.), DHARMAFECT 1™ (Dharmacon, Lafayette, Colo.), DHARMAFECT 2™ (Dharmacon), DHARMAFECT 3™ (Dharmacon), DHARMAFECT 4™ (Dharmacon), ESCORT™ III (Sigma, St. Louis, Mo.), and ESCORT™ IV (Sigma Chemical Co.)

In some cases, chemical delivery systems can be used to deliver the DNA molecules provided herein, for example, by using cationic transfection reagents, which include compaction of negatively charged nucleic acid by polycationic chemicals to form cationic liposome/micelle or cationic polymers. Cationic lipids used for the delivery method include, but not limited to monovalent cationic lipids, polyvalent cationic lipids, guanidine containing compounds, cholesterol derivative compounds, cationic polymers, (e.g., poly(ethylenimine), poly-L-lysine, protamine, other cationic polymers), and lipid-polymer hybrids.

In some embodiments, DNA molecules provided herein are delivered by making transient penetration in cell membrane by applying mechanical, electrical, ultrasonic, hydrodynamic, or laser-based energy so that DNA entrance into the targeted cells is facilitated. For example, a DNA molecule provided herein can be delivered by transiently disrupting cell membrane by squeezing the cell through a size-restricted channel or by other means known in the art.

The disclosure provides that the DNA molecules provided herein can be prepared as pharmaceutical compositions. It will be understood that such compositions necessarily comprise one or more active ingredients and, most often, a pharmaceutically acceptable excipient.

Relative amounts of the active ingredient (e.g. DNA molecules provided herein or cells comprising DNA molecules provided herein for transfer or transplantation into a subject), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

Formulations of the present disclosure can include, without limitation, saline, liposomes, lipid nanoparticles, polymers, peptides, proteins, cells comprising DNA molecules provided herein (e.g., for transfer or transplantation into a subject) and combinations thereof.

In the case of viral particles, exosomes or hybridosomes, which may contain endogenous nucleic acids, quantification of DNA molecules may be used as the measure of the dose contained in the formulation. Any method known in the art can be used to determine the DNA molecule number of a composition provided herein. One method for performing DNA molecule number titration is as follows: samples of viral particles, exosomes or hybridosomes compositions comprising hairpin-ended DNA encoding FVIII are first treated with DNase to eliminate contaminating host DNA from the production process. The DNase resistant particles are then subjected to heat treatment to release the genome from the capsid. The released genomes are then quantitated by real-time PCR using primer/probe sets targeting specific region of the viral genome (for example poly A signal). Another suitable method for determining genome copies is quantitative-PCR (qPCR), particularly the optimized qPCR or digital droplet PCR.

Formulations of the pharmaceutical compositions described herein may be prepared by any method known or hereafter developed in the art of pharmacology. As used herein the term “pharmaceutical composition” refers to compositions comprising at least one active ingredient and optionally one or more pharmaceutically acceptable excipients.

In general, such preparatory methods include the step of associating the active ingredient with an excipient and/or one or more other accessory ingredients. As used herein, the phrase “active ingredient” generally refers to either DNA molecules provided herein or cells or substance comprising the DNA molecules provided herein.

Formulations of the DNA molecules and pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

In some embodiments, the formulations described herein may contain sufficient DNA molecules or active ingredients for expression of the ORFs (e.g. encoding a human FVIII or a catalytically active fragment thereof) in the expression cassette for the treatment of a disease (e.g., Hemophilia A).

In some embodiments, DNA molecules of the present disclosure are substantially free of any viral proteins (e.g. AAV Rep78 or NS1). In some embodiments, the isolated DNA molecules of the disclosure are 100% free, 99% free, 98% free, 97% free, 96% free, 95% free, 94% free, 93% free, 92% free, 91% free, or 90% free of viral proteins.

The DNA molecules of the present disclosure can be formulated using one or more excipients or diluents to (1) increase stability; (2) increase cell transfection or transduction; (3) permit the sustained or delayed release of the active ingredients; (4) alter the biodistribution (e.g., target the DNA molecules or active ingredients comprising the DNA molecules to specific tissues or cell types); (5) increase the translation of ORFs encoding a human FVIII or a catalytically active fragment thereof in the expression cassette; (6) alter the release profile of the protein encoded by the ORFs of the expression cassette and/or (7) allow for regulatable expression of the ORFs of the expression cassette.

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, MD, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include those known and used in the art (see Remington: The Science and Practice of Pharmacy, 21st Edition, A. R. Gennaro, Lippincott, Williams & Wilkins, Baltimore, MD, 2006.)

In some embodiments, the pharmaceutical composition for the DNA molecules provided herein can comprise at least one inactive ingredient. As used herein, the term “inactive ingredient” refers to one or more agents that do not contribute to the activity of the active ingredient of the pharmaceutical composition included in formulations. In some embodiments, all, none, or some of the inactive ingredients used in the formulations of the present disclosure can be any one of such approved by the US Food and Drug Administration (FDA) and used in the art.

### 5.8 Method of Using

The disclosure provides that the DNA molecules provided herein can be used to deliver the ORFs or transgenes (e.g. encoding a human FVIII or a catalytically active fragment thereof) in the expression cassette to a cell for expression. ORFs or transgenes as described in Section 5.4.3 can be efficiently delivered. The disclosure provides that the DNA molecules provided herein can be used to deliver the ORFs or transgenes in the expression cassette to a human subject. Any ORFs or transgenes as described in Section 5.4.3 can be efficiently delivered.

In one specific embodiment, the method of delivering a gene of interest to a cell for expression comprises: transfecting the DNA molecules provided herein into the cell. In certain embodiments, the cell is a human cell. In another embodiment, the cell is a human primary cell. In yet another embodiment, the cell is a primary human blood cell. In one embodiment, the DNA molecules can be transfected into the cell via any delivery vehicles described in Section 5.7.

In another specific embodiment, the method of delivering a gene of interest to a human subject for expression comprises: transfecting the DNA molecules provided herein into a cell and administering the cell to a human subject. In certain embodiments, the cell is a human cell. In another embodiment, the cell is a human primary cell. In yet another embodiment, the cell is a primary human blood cell. In one embodiment, the DNA molecules can be transfected into the cell via any delivery vehicles described in Section 5.7.

In some embodiments, the DNA molecules provided herein can be used in gene therapy by delivering a disease correcting genes in the expression cassette into a cell or a human subject as described in the preceding 3 paragraphs.

In certain embodiments, the DNA molecules provided herein can be used to transfect cells that are difficult to transfect as known in the art. Such cells known to be difficult to transfect include cells that are not actively dividing. In some embodiments, such cells can be human primary cells, including, for example, human primary blood cells, human primary hepatocyte, human primary neurons, human primary muscle cells, human primary cardiomyocyte, or human primary liver sinusoidal endothelial cells.

### 5.8.1 Host Cell

As used herein, the term “host cell”, includes any cell type that is susceptible to transformation, transfection, transduction, and the like with a nucleic acid construct or hairpin ended expression vector of the present disclosure.

In some embodiments, a hairpin ended vector for expression of FVIII protein as disclosed herein delivers the therapeutic FVIII protein transgene into a subject host cell. In some embodiments, the subject host cell is a human host cell, including, for example blood cells, stem cells, hematopoietic cells, CD34+ cells, liver cells, cancer cells, vascular cells, muscle cells, pancreatic cells, neural cells, ocular or retinal cells, epithelial or endothelial cells, dendritic cells, fibroblasts, or any other cell of mammalian origin, including, without limitation, hepatic (i.e., liver) cells, lung cells, cardiac cells, pancreatic cells, intestinal cells, diaphragmatic cells, renal (i.e., kidney) cells, neural cells, blood cells, bone marrow cells, or any one or more selected tissues of a subject for which gene therapy is contemplated. In one aspect, the subject host cell is a human host cell.

The present disclosure also relates to recombinant host cells as mentioned above, including a hairpin ended vector for expression of FVIII protein as disclosed herein. Thus, one can use multiple host cells depending on the purpose as is obvious to the skilled artisan. A hairpin ended vector for expression of FVIII protein as disclosed herein can be introduced into a host cell so that the donor sequence is maintained as a chromosomal integrant. The term host cell encompasses any progeny of a parent cell that is not identical to the parent cell due to mutations that occur during replication. The choice of a host cell will to a large extent depend upon the donor sequence and its source.

The host cell may also be a eukaryote, such as a mammalian, insect, plant, or fungal cell. In one embodiment, the host cell is a human cell (e.g., a primary cell, a stem cell, or an immortalized cell line). In some embodiments, the host cell can be administered a hairpin ended vector for expression of FVIII protein as disclosed herein ex vivo and then delivered to the subject after the gene therapy event. A host cell can be any cell type, e.g., a somatic cell or a stem cell, an induced pluripotent stem cell, or a blood cell, e.g., T-cell or B-cell, or bone marrow cell. In certain embodiments, the host cell is an allogenic cell. In some embodiments, gene modified host cells, e.g., bone marrow stem cells, e.g., CD34 cells, or induced pluripotent stem cells can be transplanted back into a patient for expression of a therapeutic protein.

FVIII is predominantly expressed in the liver, heart, skeletal muscles, and thyroid. During fetal development, FVIII can be expressed in the adrenal gland, heart, intestine, kidney lung, and stomach. Accordingly, in some embodiments, one can administer a hairpin ended vector expressing FVIII to any one or more tissues selected from: liver, kidneys, gallbladder, prostate, adrenal. In some embodiments, when a hairpin ended vector expressing FVIII is administered to an infant, or administered to a subject in utero, one can administer a hairpin ended vector expressing FVIII to any one or more tissues selected from: liver, skeletal muscle, heart, tongue, lung, and stomach.

In some embodiments, a hairpin-ended DNA molecule for expression of FVIII protein as disclosed herein can be used to deliver an FVIII protein to skeletal, cardiac or diaphragm muscle, for production of an FVIII protein for secretion and circulation in the blood or for systemic delivery to other tissues to treat, ameliorate, and/or prevent Hemophilia A.

In other embodiments herein, the term host cell refers to cultures of liver or muscle cells of various mammalian species for in vitro assessment of the compositions described herein. Still in other embodiments, the term “host cell” is intended to reference the liver cells or muscle of the subject being treated in vivo for Hemophilia A disease.

### 5.8.2 Testing for Successful Gene Expression Using a Hairpin-Ended DNA Molecule

Assays well known in the art can be used to test the efficiency of gene delivery of an FVIII protein by a hairpin-ended DNA molecule can be performed in both in vitro and in vivo models. Levels of the expression of the FVIII protein by the hairpin-ended DNA can be assessed by one skilled in the art by measuring mRNA and protein levels of the FVIII protein (e.g., reverse transcription PCR, western blot analysis, and enzyme-linked immunosorbent assay (ELISA)). In one embodiment, the DNA comprises a reporter protein that can be used to assess the expression of the FVIII protein, for example by examining the expression of the reporter protein by fluorescence microscopy or a luminescence plate reader. For in vivo applications, protein function assays can be used to test the functionality of a given FVIII protein to determine if gene expression has successfully occurred. One skilled in the art will be able to determine the best test for measuring functionality of an FVIII protein expressed by the hairpin-ended DNA molecule in vitro or in vivo.

It is contemplated herein that the effects of gene expression of an FVIII protein from the hairpin-ended DNA in a cell or subject can last for at least 0.5 month, at least 1 month, at least 2 months, at least 3 months, at least four months, at least 5 months, at least six months, at least 10 months, at least 12 months, at least 18 months, at least 2 years, at least 5 years, at least 10 years, at least 20 years, or can be permanent.

In some embodiments, the expression cassette of hairpin-ended DNA molecules described herein can be codon optimized for the host cell. As used herein, the term “codon optimized” or “codon optimization” refers to the process of modifying a nucleic acid sequence for enhanced expression in the cells of the vertebrate of interest, e.g., mouse or human (e.g., humanized), by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's Gene Forge® codon optimization and custom gene synthesis platform (Aptagen, Inc.) or another publicly available database.

### 5.9 Methods of Treatment

In another aspect, provided herein are methods for treating a disease associated with reduced activity of coagulation factor VIIII (FVIII) in a patient, the method comprising administering to the patient a DNA molecule comprising a transgene encoding human FVIII or a catalytically active fragment thereof. In specific embodiments, the DNA molecule is contained in a hybridosome. In a specific embodiment, the DNA molecule is contained in a lipid nanoparticle.

The DNA molecules may be contained in a single vector or in multiple vectors which are co-administered.

In some embodiments, the patient treated in accordance with the methods described herein is an adult. In some embodiments, the patient is a pediatric patient. The pediatric patient may be, for example, about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, or about 18 years old. In some embodiments, the pediatric patient is an infant. As used herein, the terms “patient” and “subject” are used interchangeably. In some embodiments, the patient is human.

In specific embodiments, the disease treated in accordance with the methods described herein is Hemophilia A.

In specific embodiments, a method of treatment described herein further comprises administering one or more additional therapies to the patient. The one or more additional therapy may be administered prior to, concurrently with, or subsequently to the DNA molecule described herein. In specific embodiments, the additional therapy is for the treatment of a disease associated with reduced activity of FVIII. In specific embodiments, the additional therapy is immunosuppressive therapy. In specific embodiments, a patient treated in accordance with the methods described herein does not receive immunosuppressive therapy. In another specific embodiment, the patient is given a second, third, or higher dose of a DNA molecule described herein without receiving immunosuppressive therapy prior to, concurrently with, and/or subsequently to the second, third, or higher dose of the DNA molecule.

The DNA molecules provided herein are also used for on-demand treatment methods. The term “on-demand treatment” refers to the administration of an isolated nucleic acid molecule provided herein, including in Section 5.5, in response to symptoms of a bleeding episode or before an activity that can cause bleeding. In one aspect, the on-demand treatment can be given to a subject when bleeding starts, such as after an injury, or when bleeding is expected, such as before surgery. In another aspect, the on-demand treatment can be given prior to activities that increase the risk of bleeding, such as contact sports.

5.9.1 Determining Efficacy by Assessing FVIII Protein Expression from the DNA Vector

Essentially any method known in the art for determining protein expression can be used to analyze expression of a FVIII protein from a hairpin-ended DNA molecule. Non-limiting examples of such methods/assays include enzyme-linked immunoassay (ELISA), affinity ELISA, ELISPOT, serial dilution, flow cytometry, surface plasmon resonance analysis, kinetic exclusion assay, mass spectrometry, Western blot, immunoprecipitation, and PCR.

FVIII activity can be measured by any known methods in the art. A number of tests are available to assess the function of the coagulation system: activated partial thromboplastin time (aPTT) test, chromogenic assay, ROTEM assay, prothrombin time (PT) test (also used to determine INR), fibrinogen testing (often by the Clauss method), platelet count, platelet function testing (often by PFA-100), TCT, bleeding time, mixing test (whether an abnormality corrects if the patient's plasma is mixed with normal plasma), coagulation factor assays, antiphospholipid antibodies, D-dimer, genetic tests (e.g., factor V Leiden, prothrombin mutation G20210A), dilute Russell's viper venom time (dRVVT), miscellaneous platelet function tests, thromboelastography (TEG or Sonoclot), thromboelastometry (TEM®, e.g., ROTEM®), or euglobulin lysis time (ELT).

For assessing FVIII protein expression in vivo, a biological sample can be obtained from a subject for analysis. Exemplary biological samples include a biofluid sample, a body fluid sample, blood (including whole blood), serum, plasma, urine, saliva, a biopsy and/or tissue sample etc. A biological sample or tissue sample can also refer to a sample of tissue or fluid isolated from an individual including, but not limited to, tumor biopsy, stool, spinal fluid, pleural fluid, nipple aspirates, lymph fluid, the external sections of the skin, respiratory, intestinal, and genitourinary tracts, tears, saliva, breast milk, cells (including, but not limited to, blood cells), tumors, organs, and also samples of in vitro cell culture constituent. The term also includes a mixture of the above-mentioned samples. The term “sample” also includes untreated or pretreated (or pre-processed) biological samples. In some embodiments, the sample used for the assays and methods described herein comprises a serum sample collected from a subject to be tested.

### 5.9.2 Determining Efficacy of the Expressed FVIII Protein by Clinical Parameters

The efficacy of a given FVIII protein expressed by a hairpin-ended DNA molecule for Hemophilia A (i.e., functional expression) can be determined by the skilled clinician. However, a treatment is considered “effective treatment,” as the term is used herein, if any one or all of the signs or symptoms of Hemophilia A is/are altered in a beneficial manner, or other clinically accepted symptoms or markers of disease are improved, or ameliorated, e.g., by at least 10% following treatment with a DNA vector described herein, encoding a therapeutic FVIII protein as described herein. Efficacy can also be measured by failure of an individual to worsen as assessed by stabilization of Hemophilia A, or the need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting Hemophilia A, e.g., arresting, or slowing progression of Hemophilia A; or (2) relieving the Hemophilia A, e.g., causing regression of Hemophilia A symptoms; and (3) preventing or reducing the likelihood of the development of the Hemophilia A disease, or preventing secondary diseases/disorders associated with Hemophilia A. An effective amount for the treatment of a disease means that amount which, when administered to a mammal in need thereof, is sufficient to result in effective treatment as that term is defined herein, for that disease. Efficacy of an agent can be determined by assessing physical indicators that are particular to Hemophilia A disease. A physician can assess for any one or more of clinical symptoms of Hemophilia A which include: unusual bleeding with minor or no trauma, arthralgia, oral cavity bleeding and spontaneous hematomas. Furthermore, biochemical characteristics are low FVIII activity and prolonged coagulation time.

In some embodiments, a method of treatment described herein results in a reduction in the number of events during which blood FVIII levels are below 0.01 IU/mL, below 0.05 IU/mL, or below 0.1 IU/mL in a subject.

In one embodiment, the methods of treatment described herein result in maintaining, e.g., a FVIII trough level of at least about 1 IU/dL, 2 IU/dL, 3 IU/dL, 4 IU/dL, 5 IU/dL, 6 IU/dL, 7 IU/dL, 8 IU/dL, 9 IU/dL, 10 IU/dL, 11 IU/dL, 12 IU/dL, 13 IU/dL, 14 IU/dL, 15 IU/dL, 16 IU/dL, 17 IU/dL, 18 IU/dL, 19 IU/dL, 20 IU/dL, 25 IU/dL, 30 IU/dL, 35 IU/dL, 40 IU/dL, 45 IU/dL, 50 IU/dL, 55 IU/dL, 60 IU/dL, 65 IU/dL, 70 IU/dL, 75 IU/dL, 80 IU/dL, 85 IU/dL, 90 IU/dL, 95 IU/dL, 100 IU/dL, 105 IU/dL, 110 IU/dL, 115 IU/dL, 120 IU/dL, 125 IU/dL, 130 IU/dL, 135 IU/dL, 140 IU/dL, 145 IU/dL, or 150 IU/dL in a subject by administering an hairpin ended nucleic acid molecule provided herein, including in Section 5.5. In one embodiment, the methods of treatment described herein result in maintaining, e.g., a FVIII trough level of at least about 1 and about 150 IU/dL, about 1 and about 125 IU/dL, about 1 and about 100 IU/dL, about 1 and about 90 IU/dL, about 1 and about 85 IU/dL, about 1 and about 80 IU/dL, about 1 and about 75 IU/dL, about 1 and about 70 IU/dL, about 1 and about 65 IU/dL, about 1 and about 60 IU/dL, about 1 and about 55 IU/dL, about 1 and about 50 IU/dL, about 1 and about 45 IU/dL, about 1 and about 40 IU/dL, about 1 and about 35 IU/dL, about 1 and about 30 IU/dL, about 1 and about 25 IU/dL, about 25 and about 125 IU/dL, about 50 and about 100 IU/dL, about 50 and about 75 IU/dL, about 75 and about 100 IU/dL, about 1 and about 20 IU/dL, about 2 and about 20 IU/dL, about 3 and about 20 IU/dL, about 4 and about 20 IU/dL, about 5 and about 20 IU/dL, about 6 and about 20 IU/dL, about 7 and about 20 IU/dL, about 8 and about 20 IU/dL, about 9 and about 20 IU/dL, or about 10 and about 20 IU/dL. Treatment or treating of a disease or condition can also include maintaining FVIII activity in a subject at a level comparable to at least about 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, or 150% of the FVIII activity in a non-hemophiliac subject. The minimum trough level required for treatment can be measured by one or more known methods and can be adjusted (increased or decreased) for each person. In one aspect, a coding sequence is provided which encodes a functional FVIII protein. By “functional FVIII”, is meant a gene which encodes an FVIII protein which provides at least about 50%, at least about 75%, at least about 80%, at least about 90%, or about the same, or greater than 100% of the biological activity level of the native FVIII protein, or a natural variant or polymorph thereof which is not associated with disease. Activity levels can be measured or determined by any art-recognized method for determining activity levels in biological samples. The term “activity level” as used herein, preferably means the activity of the protein per volume, mass, or weight of sample or total protein within a sample. In exemplary embodiments, the “activity level” is described in terms of units per milliliter of fluid (e.g., bodily fluid, e.g., serum, plasma, urine, and the like) or is described in terms of units per weight of tissue or per weight of protein (e.g., total protein) within a sample. Factor VIII is a component of the coagulation pathway and can be functionally characterized in terms of clotting or bleeding propensity or biologically characterized by binding assays such as ELISA.

In some embodiments the hairpin-ended DNA molecules encoding a functional FVIII protein can be delivered to the liver cells, of a patient in need (e.g., a Hemophilia A patient), and can elevate active FVIII levels of the patient. The hairpin-ended DNA molecule can be used for preventing, treating, ameliorating, or reversing any symptoms of Hemophilia A in the patient.

In further aspects, a hairpin-ended DNA molecule of this disclosure can also be used for reducing the dependence of a Hemophilia A patient on a particular enzyme replacement to control the disease. For instance, a hairpin-ended DNA molecule of this invention can be used to reduce a Hemophilia A patient's dependence on frequent infusions of therapeutic Factor VIII.

### 5.9.3 Administration

In one aspect, the DNA molecule provided herein, including in Section 5.5 may be administered to a subject once or repeatedly. Thus, in specific embodiments, a method for treating a disease associated with reduced activity of FVIII in a human patient comprises the steps of (i) administering a first dose of a DNA molecule comprising an expression cassette comprising a transgene encoding human FVIII or a catalytically active fragment thereof to the patient and (ii) administering a second dose of the DNA molecule to the patient.

In some embodiments, the first dose of the DNA molecule is administered to the patient at least one month, at least two months, at least 3 months, at least 4 months, at least 5 months, at least 6 months, at least 7 months, at least 8 months, at least 9 months, at least 10 months, or at least 11 months before the second dose of the DNA molecule. In some embodiments, the first dose of the DNA molecule is administered to the patient at least 1 year, at least 2 years, at least 3 years, at least 4 years, at least 5 years, at least 10 years, at least 15 years, or at least 20 years before the second dose of the DNA molecule.

In some embodiments, the first dose of the DNA molecule is administered about 1-3 months, about 3-6 months, about 6-9 months, about 9-12 months, about 12-15 months, about 15-18 months, about 18-21 months, about 21-24 months, about 24-27 months, about 27-30 months, about 30-33 months, about 33-36 months, about 3-4 years, about 4-5 years, about 5-6 years, about 6-7 years, about 8-9 years, about 9-10 yeasts, about 10-11 years, about 11-12 years, about 12-13 years, about 13-14 years, about 14-15 years, about 15-16 years, about 16-17 years, about 17-18 years, about 18-19 years, or about 19-20 years before the second dose of the DNA molecule.

The first dose of the double-stranded DNA molecule and the second dose of the DNA molecule may contain the same amount of the DNA molecule or different amounts of the DNA molecule.

In some embodiments, a method of treatment described herein further comprises administering one or more additional doses of the DNA molecule, e.g., administering a total of 3, 4, 5, 6, 7, 8, 9, or 10 doses of the DNA molecule.

The DNA molecule may be administered once weekly, biweekly (every other week), or monthly. In some embodiments, the DNA molecule is administered about every 3 months, about every 6 months, about every 9 months, about every 12 months, about every 15 months, about every 18 months, about every 21 months, about every 2 years, about every 3 years, about every 4 years, about every 5 years, about every 6 years, about every 7 years, about every 8 years, about every 9 years, about every 10 years, about every 11 years, about every 12 years, about every 13 years, about every 14 years, about every 15 years, about every 16 years, about every 17 years, about every 18 years, about every 19 years, or about every 20 years.

In specific embodiments, the DNA molecule is administered to the patient for the duration of the life of the patient.

A DNA molecule described herein may be administered to a subject by any suitable route. In certain embodiments, said route of administration is selected from the group consisting of intravenous, intravascular, intraarterial, intramuscular, intraocular, subcutaneous, and intradermal. In a specific embodiment, said route is intravenous. In other embodiments, said route is an administration route delivering the hairpin-ended DNA to the liver that is other than intravenous, intravascular, intraarterial, intramuscular, intraocular, subcutaneous, and intradermal.

In some embodiments, a method of treating a disease in a subject comprises introducing into a target cell in need thereof (in particular a muscle cell or tissue) of the subject, a therapeutically effective amount of a hairpin ended molecule encoding a FVIII protein, optionally with a pharmaceutically acceptable carrier. In some embodiments, the hairpin-ended DNA molecule for expression of FVIII protein, is administered to a muscle tissue of a subject.

In some embodiments, administration of the hairpin-ended DNA molecule can be to any site in a subject, including, without limitation, a site selected from the group consisting of a smooth muscle, skeletal muscle, the heart, the diaphragm, or muscles of the eye.

Administration of a hairpin-ended DNA molecule for expression of FVIII protein as disclosed herein, to a skeletal muscle according to the present disclosure includes but is not limited to administration to the skeletal muscle in the limbs (e.g., upper leg, lower leg, upper arm and/or lower arm), thorax, abdomen, back, neck, head (e.g., tongue), pelvis/perineum, and/or digits. The hairpin-ended DNA molecule as disclosed herein can be delivered to skeletal muscle by intravenous administration, intra-arterial administration, intraperitoneal administration, limb perfusion, (optionally, isolated limb perfusion of a leg and/or arm; see, e.g. Arruda et al., (2005) Blood 105:3458-3464), and/or direct intramuscular injection. In particular embodiments, the hairpin-ended DNA molecule encoding FVIII as disclosed herein is administered to the liver, eye, a limb (e.g., arm and/or leg) of a subject (e.g., a subject with Hemophilia A) by limb perfusion, optionally isolated limb perfusion (e.g., by intravenous or intra-articular administration.

Furthermore, a composition comprising a hairpin-ended DNA molecule for expression of FVIII protein, as disclosed herein, which is administered to a skeletal muscle, can be administered to a skeletal muscle in the limbs (e.g., upper leg, lower leg, upper arm and or lower arm,), thorax, abdomen, back, neck, head (e.g., tongue), pelvis/perineum, and/or digits. Suitable skeletal muscles include but are not limited to abductor digiti minimi (in the hand), abductor digiti minimi (in the foot), abductor hallucis, abductor ossis metatarsi quinti, abductor pollicis brevis, abductor pollicis longus, adductor brevis, adductor hallucis, adductor longus, adductor magnus, adductor pollicis, anconeus, anterior scalene, articularis genus, biceps brachii, biceps femoris, brachialis, brachioradialis, buccinator, coracobrachialis, corrugator supercilii, deltoid, depressor anguli oris, depressor labii inferioris, digastric, dorsal interossei (in the hand), dorsal interossei (in the foot), extensor carpi radialis brevis, extensor carpi radialis longus, extensor carpi ulnaris, extensor digiti minimi, extensor digitorum, extensor digitorum brevis, extensor digitorum longus, extensor hallucis brevis, extensor hallucis longus, extensor indicis, extensor pollicis brevis, extensor pollicis longus, flexor carpi radialis, flexor carpi ulnaris, flexor digiti minimi brevis (in the hand), flexor digiti minimi brevis (in the foot), flexor digitorum brevis, flexor digitorum longus, flexor digitorum profundus, flexor digitorum superficialis, flexor hallucis brevis, flexor hallucis longus, flexor pollicis brevis, flexor pollicis longus, frontalis, gastrocnemius, geniohyoid, gluteus maximus, gluteus medius, gluteus minimus, gracilis, iliocostalis cervicis, iliocostalis lumborum, iliocostalis thoracis, illiacus, inferior gemellus, inferior oblique, inferior rectus, infraspinatus, inter spinalis, intertransversi, lateral pterygoid, lateral rectus, latissimus dorsi, levator anguli oris, levator labii superioris, levator labii superioris alaeque nasi, levator palpebrae superioris, levator scapulae, long rotators, longissimus capitis, longissimus cervicis, longissimus thoracis, longus capitis, longus colli, lumbricals (in the hand), lumbricals (in the foot), masseter, medial pterygoid, medial rectus, middle scalene, multifidus, mylohyoid, obliquus capitis inferior, obliquus capitis superior, obturator externus, obturator internus, occipitalis, omohyoid, opponens digiti minimi, opponens pollicis, orbicularis oculi, orbicularis oris, palmar interossei, palmaris brevis, palmaris longus, pectineus, pectoralis major, pectoralis minor, peroneus brevis, peroneus longus, peroneus tertius, piriformis, plantar interossei, plantaris, platysma, popliteus, posterior scalene, pronator quadratus, pronator teres, psoas major, quadratus femoris, quadratus plantae, rectus capitis anterior, rectus capitis lateralis, rectus capitis posterior major, rectus capitis posterior minor, rectus femoris, rhomboid major, rhomboid minor, risorius, sartorius, scalenus minimus, semimembranosus, semispinalis capitis, semispinalis cervicis, semispinalis thoracis, semitendinosus, serratus anterior, short rotators, soleus, spinalis capitis, spinalis cervicis, spinalis thoracis, splenius capitis, splenius cervicis, sternocleidomastoid sternohyoid, sternothyroid, stylohyoid, subclavius, subscapularis, superior gemellus, superior oblique, superior rectus, supinator, supraspinatus, temporalis, tensor fascia lata, teres major, teres minor, thoracis, thyrohyoid, tibialis anterior, tibialis posterior, trapezius, triceps brachii, vastus intermedius, vastus lateralis, vastus medialis, zygomaticus major, and zygomaticus minor, and any other suitable skeletal muscle as known in the art.

In certain embodiments Administration of a hairpin-ended DNA molecule for the expression of FVIII protein, as disclosed herein, to diaphragm muscle can be by any suitable method including intravenous administration, intra-arterial administration, and/or intra-peritoneal administration.

Administration of a hairpin-ended DNA molecule for expression of FVIII protein as disclosed herein to cardiac muscle includes administration to the left atrium, right atrium, left ventricle, right ventricle and/or septum. The hairpin-ended DNA molecule as described herein can be delivered to cardiac muscle by intravenous administration, intra-arterial administration such as intra-aortic administration, direct cardiac injection (e.g., into left atrium, right atrium, left ventricle, right ventricle), and/or coronary artery perfusion.

Administration of a hairpin-ended DNA molecule for expression of FVIII protein as disclosed herein to smooth muscle can be by any suitable method including intravenous administration, intra-arterial administration, and/or intra-peritoneal administration. In one embodiment, administration can be to endothelial cells present in, near, and/or on smooth muscle. Non-limiting examples of smooth muscles include the iris of the eye, bronchioles of the lung, laryngeal muscles (vocal cords), muscular layers of the stomach, esophagus, small and large intestine of the gastrointestinal tract, ureter, detrusor muscle of the urinary bladder, uterine myometrium, penis, or prostate gland.

In some embodiments, a hairpin-ended DNA molecule for expression of FVIII protein as disclosed herein is administered to skeletal muscle, diaphragm muscle and/or cardiac muscle. In representative embodiments, a hairpin-ended DNA molecule according to the present disclosure is used to treat and/or prevent disorders of skeletal, cardiac and/or diaphragm muscle.

In some embodiments, a composition comprising a hairpin-ended DNA molecule for expression of FVIII protein, as disclosed herein, can be injected into one or more sites of a given muscle, for example, skeletal muscle (e.g., deltoid, vastus lateralis, ventrogluteal muscle of dorsogluteal muscle, or anterolateral thigh for infants) in a subject using a needle. In certain embodiments, the composition comprising hairpin-ended DNA molecule can be introduced to other subtypes of muscle cells. Non-limiting examples of muscle cell subtypes include skeletal muscle cells, cardiac muscle cells, smooth muscle cells and/or diaphragm muscle cells.

In certain embodiments, the compositions are delivered to multiple sites in one or more muscles of the subject. For example, the composition may be delivered by injections in at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100 injections sites. Such sites can be spread over the area of a single muscle or can be distributed among multiple muscles.

In some embodiments, delivery of an expressed transgene from the hairpin-ended DNA molecule to a target tissue can also be achieved by delivering a synthetic depot comprising the hairpin-ended DNA molecule, where a depot comprising the hairpin-ended DNA molecule is implanted into skeletal, smooth, cardiac and/or diaphragm muscle tissue or the muscle tissue can be contacted matrix comprising the hairpin-ended DNA molecule, as described herein. Such implantable matrices or substrates are described in U.S. Pat. No. 7,201,898, incorporated by reference in its entirety herein.

Methods for intramuscular injection are known to those of skill in the art and as such are not described in detail herein. However, when performing an intramuscular injection, an appropriate needle size should be determined based on the age and size of the patient, the viscosity of the composition, as well as the site of injection.

In certain embodiments, a hairpin-ended DNA molecule for expression of FVIII protein as disclosed herein is administered in the absence of a carrier to facilitate entry of hairpin-ended DNA molecule into the cells, or in a physiologically inert pharmaceutically acceptable carrier (i.e., any carrier that does not improve or enhance uptake of the capsid free, non-viral vectors into the myotubes). In such embodiments, the uptake of the hairpin-ended DNA molecule for expression of FVIII protein can be facilitated by electroporation of the cell or tissue. With electroporation, electrical fields are used to create pores in cells without causing permanent damage to the cells. These pores are large enough to allow hairpin-ended DNA molecule for expression of FVIII to gain access to the interior of the cell. Over time, the pores in the cell membrane close and the cell once again becomes impermeable.

There are a number of methods for in vivo electroporation; electrodes can be provided in various configurations such as, for example, a caliper that grips the epidermis overlying a region of cells to be treated. Alternatively, needle-shaped electrodes may be inserted into the tissue, to access more deeply located cells. In either case, after the composition comprising e.g., hairpin-ended DNA molecule for expression of FVIII are injected into the treatment region, the electrodes apply an electrical field to the region.

In another embodiment, a hairpin-ended DNA molecule for expression of FVIII protein is administered to the liver. The hairpin-ended DNA may also be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes, cortex, basal ganglia, hippocampus, and porta amygdala), limbic system, neocortex, corpus striatum, cerebrum, and inferior colliculus. The hairpin-ended DNA vector may be delivered into the cerebrospinal fluid (e.g., by lumbar puncture). The hairpin-ended DNA for expression of FVIII protein may further be administered intravascularly to the CNS in situations in which the blood-brain barrier has been perturbed (e.g., brain tumor or cerebral infarct).

In some embodiments, the hairpin-ended DNA for expression of FVIII protein can be administered in a liquid formulation by direct injection (e.g., stereotactic injection) to the desired region or compartment in the CNS. In other embodiments, the hairpin-ended DNA molecule can be provided by topical application to the desired region or by intranasal administration of an aerosol formulation.

### 5.9.4 Dosing

Provided herein are methods of treatment comprising administering to the subject an effective amount of a composition comprising a hairpin ended vector encoding an FVIII protein as described herein. As will be appreciated by a skilled practitioner, the term “effective amount” refers to the amount of the hairpin-ended DNA molecule composition administered which results in expression of the FVIII protein in a “therapeutically effective amount” for the treatment of a disease or a disorder associated with reduced presence or function of FVIII in a subject (e.g. Hemophilia A).

In vivo and/or in vitro assays can optionally be employed to help identify optimal dosage ranges for use. The precise dose to be employed in the formulation will also depend on the route of administration, and the seriousness of the condition, and should be decided according to the judgment of the person of ordinary skill in the art and each subject's circumstances. Effective doses can be extrapolated from dose-response curves derived from in vitro or animal model test systems, (e.g. patient derived fibroblasts, murine or canine models)

Hairpin ended vectors for expression of FVIII protein as disclosed herein, can be administered in sufficient amounts to transfect the cells of a desired tissue and to provide sufficient levels of gene expression without undue adverse effects. It is desirable that the lowest effective concentration hairpin ended vector encoding FVIII be utilized in order to reduce the risk of undesirable effects, such as toxicity. In some embodiments other dosages in these ranges may be selected by the attending physician, taking into account the physical state of the subject, preferably human, being treated, the age of the subject, and the degree to which the disorder, has developed. Conventional and pharmaceutically acceptable routes of administration include, but are not limited to, those described above in the “Administration” section, such as direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration can be combined, if desired.

In certain embodiments, the amount (i.e. dose) of a hairpin ended vectors for expression of FVIII protein as disclosed herein required to achieve a particular “therapeutic effect,” will vary based on several factors including, but not limited to: the route of nucleic acid administration, the pharmaceutical carrier, the level of gene expression required to achieve a therapeutic effect, the specific disease or disorder being treated, and the stability of the gene(s), RNA product(s), or resulting expressed protein(s). One of skill in the art can readily determine a hairpin ended vector dose range to treat a patient having a disease or a disorder associated to reduced presence or function of FVIII in a subject (e.g. Hemophilia A) based on the aforementioned factors, as well as other factors that are well known in the art.

In general, the dosage regime can be adjusted to provide the optimum therapeutic response. For example, the hairpin ended vectors for expression of FVIII protein can be repeatedly administered, e.g., several doses can be administered daily, or the dose can be proportionally reduced as indicated by the exigencies of the therapeutic situation. One of ordinary skill in the art will readily be able to determine appropriate doses and schedules of administration of the subject vectors described herein as well as whether the said vectors are to be administered to cells or to subjects.

A “therapeutically effective dose” will fall in a relatively broad range that can be determined through clinical trials and will depend on the particular application (for example, direct ocular injections require very small amounts, while systemic injection would require large amounts). For example, for direct in vivo injection into skeletal or cardiac muscle of a human subject, a therapeutically effective dose will be on the order of from about 1 μg to 100 g of the hairpin-ended DNA molecule. If exosomes or hybridosomes are used to deliver the hairpin-ended DNA molecule vector, then a therapeutically effective dose can be determined experimentally, but is expected to deliver from 1 μg to about 100 g of vector. Moreover, a therapeutically effective dose is an amount hairpin-ended DNA molecule that expresses a sufficient amount of the transgene to have an effect on the subject that results in a reduction in one or more symptoms of the disease, but does not result in significant off-target or significant adverse side effects. In one embodiment, a “therapeutically effective amount” is an amount of an expressed FVIII protein that is sufficient to produce a statistically significant, measurable change in expression of Hemophilia A biomarker or reduction of a given disease symptom. Such effective amounts can be gauged in clinical trials as well as animal studies for a given hairpin-ended DNA molecule composition. In some embodiments, a transgene encodes a catalytically active fragment of FVIII. A “catalytically active fragment of FVIII” is any truncated form of FVIII which retains its catalytic functions.

Formulation of pharmaceutically acceptable excipients and carrier solutions is well-known to those of skill in the art, as is the development of suitable dosing and treatment regimens for using the particular compositions described herein in a variety of treatment regimens.

For in vitro transfection, an effective amount of a hairpin-ended DNA molecule vectors for expression of FVIII protein as disclosed herein to be delivered to cells (1×10⁶ cells) will be on the of 0.1 to 100 μg hairpin-ended DNA molecule vector, preferably 1 to 20 μg, and more preferably 1 to 15 μg or 8 to 10 μg. Larger hairpin-ended DNA molecule vectors will require higher doses. If Hybridosomes, exosomes or lipid nanoparticles are used, an effective in vitro dose can be determined experimentally but would be intended to deliver generally the same amount of the hairpin-ended DNA molecule vector.

For the treatment of Hemophilia A, the appropriate dosage of a hairpin-ended DNA molecule vector that expresses an FVIII protein as disclosed herein will depend on the specific type of disease to be treated, the type of a FVIII protein, the severity and course of the Hemophilia A disease, previous therapy, the patient's clinical history and response to the vector, and the discretion of the attending physician. The hairpin-ended DNA molecule vector encoding a FVIII protein is suitably administered to the patient at one time or over a series of treatments. Various dosing schedules including, but not limited to, single or multiple administrations over various time-points, bolus administration, and pulse infusion are contemplated herein.

Depending on the type and severity of the disease or disorder, a hairpin-ended DNA molecule vector is administered in an amount that the encoded FVIII protein is expressed at about 0.3 mg/kg to 100 mg/kg (e.g. 15 mg/kg-100 mg/kg, or any dosage within that range), by one or more separate administrations, or by continuous infusion. One typical daily dosage of the hairpin-ended DNA molecule is sufficient to result in the expression of the encoded FVIII protein at a range from about 15 mg/kg to 100 mg/kg or more, depending on the factors mentioned above. One exemplary dose of the hairpin-ended DNA molecule is an amount sufficient to result in the expression of the encoded FVIII protein as disclosed herein in a range from about 10 mg/kg to about 50 mg/kg. Thus, one or more doses of a hairpin-ended DNA molecule in an amount sufficient to result in the expression of the encoded FVIII protein at about 0.5 mg/kg, 1 mg/kg, 1.5 mg/kg, 2.0 mg/kg, 3 mg/kg, 4.0 mg/kg, 5 mg/kg, 10 mg/kg, 15 mg/kg, 20 mg/kg, 25 mg/kg, 30 mg/kg, 35 mg/kg, 40 mg/kg, 50 mg/kg, 60 mg/kg, 70 mg/kg, 80 mg/kg, 90 mg/kg, or 100 mg/kg (or any combination thereof) may be administered to the patient.

In some embodiments, a therapeutically effective dose of a hairpin-ended DNA encoding FVIII in vivo can be a dose of about 0.001 to about 500 mg/kg body weight. For instance, the therapeutically effective dose may be about 0.001-0.01 mg/kg body weight, or 0.01-0.1 mg/kg, or 0.1-1 mg/kg, or 1-10 mg/kg, or 10-100 mg/kg. In some embodiments, a hairpin-ended DNA molecule encoding FVIII is provided at a dose ranging from about 0.1 to about 10 mg/kg body weight, e.g., from about 0.5 to about 5 mg/kg, from about 1 to about 4.5 mg/kg, or from about 2 to about 4 mg/kg.

In another embodiment the therapeutically effective dose of an hairpin-ended DNA encoding FVIII in vivo can be a dose of at least about 0.001 mg/kg body weight, or at least about 0.01 mg/kg, or at least about 0.1 mg/kg, or at least about 1 mg/kg, or at least about 2 mg/kg, or at least about 3 mg/kg, or at least about 4 mg/kg, or at least about 5 mg/kg, at least about 10 mg/kg, at least about 20 mg/kg, at least about 50 mg/kg, or more. In some embodiments, a hairpin-ended DNA encoding FVIII is provided at a dose of about 0.1 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 5 mg/kg, or about 6, 7, 8, 9, 10, 15, 20, 25, 50, 75, or 100 mg/kg.

In some embodiments, the hairpin-ended DNA molecule is an amount sufficient to result in the expression of the encoded FVIII protein for a total dose in the range of 50 mg to 2500 mg. An exemplary dose of a hairpin-ended DNA molecule is an amount sufficient to result in the total expression of the encoded FVIII protein at about 50 mg, about 100 mg, 200 mg, 300 mg, 400 mg, about 500 mg, about 600 mg, about 700 mg, about 720 mg, about 1000 mg, about 1050 mg, about 1100 mg, about 1200 mg, about 1300 mg, about 1400 mg, about 1500 mg, about 1600 mg, about 1700 mg, about 1800 mg, about 1900 mg, about 2000 mg, about 2050 mg, about 2100 mg, about 2200 mg, about 2300 mg, about 2400 mg, or about 2500 mg (or any combination thereof). As the expression of the FVIII protein from hairpin-ended DNA molecule can be carefully controlled by regulatory switches herein, or alternatively multiple dose of the hairpin-ended DNA molecule administered to the subject, the expression of the FVIII protein from the hairpin-ended DNA molecule can be controlled in such a way that the doses of the expressed FVIII protein may be administered intermittently, e.g. every week, every two weeks, every three weeks, every four weeks, every month, every two months, every three months, or every six months from the hairpin-ended DNA molecule. The progress of this therapy can be monitored by conventional techniques and assays.

In certain embodiments, a hairpin-ended DNA molecule is administered an amount sufficient to result in the expression of the encoded FVIII protein at a dose of 15 mg/kg, 30 mg/kg, 40 mg/kg, 45 mg/kg, 50 mg/kg, 60 mg/kg, or a flat dose, e.g., 300 mg, 500 mg, 700 mg, 800 mg, or higher.

In some embodiments, the expression of the FVIII protein from the hairpin-ended DNA molecule is controlled such that the FVIII protein is expressed every day, every other day, every week, every 2 weeks, or every 4 weeks for a period of time. In some embodiments, the expression of the FVIII protein from the hairpin-ended DNA molecule is controlled such that the FVIII protein is expressed every 2 weeks or every 4 weeks for a period of time. In certain embodiments, the period of time is 6 months, one year, eighteen months, two years, five years, ten years, 15 years, 20 years, or the lifetime of the patient.

Treatment can involve administration of a single dose or multiple doses. In some embodiments, more than one dose can be administered to a subject. Without wishing to be bound by any particular theory or mechanism, comparison to viral vectors, multiple doses can be administered as needed, because the hairpin-ended DNA molecule does not elicit an anti-viral host immune response due to the absence of proteins of viral origin. As such, one of skill in the art can readily determine an appropriate number of doses. The number of doses administered can, for example, be on the order of 1-100, or on the order of 2-50 doses.

In certain embodiments, the interval between a first administration said hairpin-ended DNA via and second administration said may be about 0.5 hour, 1 hour, about 2 hours, about 3 hours, about 4 hours, about 5 hours, about 6 hours, about 7 hours, about 8 hours, about 9 hours, about 10 hours, about 11 hours, about 12 hours, about 1 day, about 2 days, about 3 days, about 4 days, about 5 days, about 6 days, about 1 week, about 8 days, about 9 days, about 10 days, about 11 days, about 12 days, about 13 days, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 1 month, about 2 months, about 3 months, about 4 months, about 5 months, about 6 months, or more.

Without wishing to be bound by any particular theory, the lack of typical anti-viral immune response (i.e., the absence anti-viral protein responses) elicited by administration of a composition comprising a hairpin-ended DNA molecule described herein allows the hairpin-ended DNA molecule for expression of FVIII protein to be administered to a host on multiple occasions. In some embodiments, the number of occasions in which a hairpin-ended DNA molecule for the expression of FVIII is delivered to a subject is in a range of 2 to 10 times (e.g., 2, 3, 4, 5, 6, 7, 8, 9, or 10 times). In some embodiments, a hairpin-ended DNA molecule is delivered to a subject more than 10 times.

In some embodiments, a dose of a hairpin-ended DNA molecule for expression of FVIII protein as disclosed herein is administered to a subject no more than once per calendar day (e.g., a 24-hour period). In some embodiments, a dose of a hairpin-ended DNA molecule is administered to a subject no more than once per 2, 3, 4, 5, 6, or 7 calendar days. In some embodiments, a dose of a hairpin-ended DNA molecule for expression of FVIII protein as disclosed herein is administered to a subject no more than once per calendar week (e.g., 1 calendar days). In some embodiments, a dose of a hairpin-ended DNA molecule is administered to a subject no more than bi-weekly (e.g., once in a two calendar week period). In some embodiments, a dose of a hairpin-ended DNA molecule is administered to a subject no more than once per calendar month (e.g., once in 30 calendar days). In some embodiments, a dose of a hairpin-ended DNA molecule is administered to a subject no more than once per six calendar months. In some embodiments, a dose of a hairpin-ended DNA molecule is administered to a subject no more than once per calendar year (e.g., 365 days or 366 days in a leap year).

In particular embodiments, more than one administration (e.g., two, three, four or more administrations) of a hairpin-ended DNA molecule for expression of FVIII protein as disclosed herein, may be employed to achieve the desired level of gene expression over a period of various intervals, e.g., daily, weekly, monthly, yearly, etc.

In some embodiments, a therapeutic a FVIII protein encoded by a hairpin-ended DNA molecule as disclosed herein can be regulated by a regulatory switch, inducible or repressible promotor so that it is expressed in a subject for at least 1 hour, at least 2 hours, at least 5 hours, at least 10 hours, at least 12 hours, at least 18 hours, at least 24 hours, at least 36 hours, at least 48 hours, at least 72 hours, at least 1 week, at least 2 weeks, at least 1 month, at least 2 months, at least 6 months, at least 12 months/one year, at least 2 years, at least 5 years, at least 10 years, at least 15 years, at least 20 years, at least 30 years, at least 40 years, at least 50 years or more. In one embodiment, the expression can be achieved by repeated administration of the hairpin-ended DNA molecules described herein at predetermined or desired intervals.

The duration of treatment depends upon the subject's clinical progress and responsiveness to therapy. In one embodiment, repeated, relatively low maintenance doses are contemplated after an initial higher therapeutic dose.

In some embodiments, the pharmaceutical compositions comprising a hairpin-ended DNA molecule for expression of FVIII protein as disclosed herein can conveniently be presented in unit dosage form. A unit dosage form will typically be adapted to one or more specific routes of administration of the pharmaceutical composition. In some embodiments, the unit dosage form is adapted for droplets to be administered directly to the eye. In some embodiments, the unit dosage form is adapted for administration by inhalation. In some embodiments, the unit dosage form is adapted for administration by a vaporizer. In some embodiments, the unit dosage form is adapted for administration by a nebulizer. In some embodiments, the unit dosage form is adapted for administration by an aerosolizer. In some embodiments, the unit dosage form is adapted for oral administration, for buccal administration, or for sublingual administration. In some embodiments, the unit dosage form is adapted for intravenous, intramuscular, or subcutaneous administration. In some embodiments, the unit dosage form is adapted for subretinal injection, suprachoroidal injection or intravitreal injection.

In some embodiments, the unit dosage form is adapted for intrathecal or intracerebroventricular administration. In some embodiments, the pharmaceutical composition is formulated for topical administration. The amount of active ingredient which can be combined with a carrier material to produce a single dosage form will generally be that amount of the compound which produces a therapeutic effect.

### 5.9.5 Outcome Assessments

A therapeutically effective dose can be administered in one or more separate administrations, and by different routes. As will be appreciated in the art, a therapeutically effective dose or a therapeutically effective amount is largely determined based on the total amount of the therapeutic agent contained in the pharmaceutical compositions of the present disclosure. Generally, a therapeutically effective amount is sufficient to achieve a meaningful benefit to the subject (e.g., treating, modulating, curing, preventing and/or ameliorating Hemophilia A). For example, a therapeutically effective amount may be an amount sufficient to achieve a desired therapeutic and/or prophylactic effect. Generally, the amount of a therapeutic agent (e.g., a hairpin-ended DNA molecule encoding FVIII) administered to a subject in need thereof will depend upon the characteristics of the subject. Such characteristics include the condition, disease severity, general health, age, sex, and body weight of the subject. One of ordinary skill in the art will be readily able to determine appropriate dosages depending on these and other related factors. In addition, both objective and subjective assays may optionally be employed to identify optimal dosage ranges.

In some embodiments, administering a therapeutically effective dose of a composition comprising a hairpin-ended DNA molecule as described herein can lead to increased liver FVIII protein levels in a treated subject. In some embodiments, administering a composition comprising a hairpin-ended DNA molecule described herein results in a 5%, 10%, 20%, 30%, 40%, 50%, 60%, 70%, 80%, 90%, or 95% increase in liver FVIII protein levels relative to a baseline FVIII protein level in the subject prior to treatment. In certain embodiments, administering a therapeutically effective dose of a composition comprising a hairpin-ended DNA molecule as described herein will result an increase in liver FVIII levels relative to baseline liver FVIII levels in the subject prior to treatment. In some embodiments, the increase in liver FVIII levels relative to baseline liver FVIII levels will be at least 5%, 10%, 20%, 30%, 40%, 50%, 100%, 200%, or more.

In some embodiments, administering a composition comprising a hairpin-ended DNA molecule described herein results in a 5%, 10%, 20%, 30%, 40%, 50%, 60%, 70%, 80%, 90%, or 95% in liver FVIII protein levels relative to a baseline FVIII protein level in the subject prior to treatment. In certain embodiments, administering a therapeutically effective dose of a composition comprising a hairpin-ended DNA molecule as described herein will result an increase in liver FVIII levels relative to baseline liver FVIII levels in the subject prior to treatment. In some embodiments, the increase in liver FVIII levels relative to baseline liver FVIII levels will be at least 5%, 10%, 20%, 30%, 40%, 50%, 100%, 200%, or more.

In some embodiments, administering a composition comprising a hairpin-ended DNA molecule described herein results in a 5%, 10%, 20%, 30%, 40%, 50%, 60%, 70%, 80%, 90%, or 95% in plasma FVIII protein levels relative to a baseline FVIII protein level in the subject prior to treatment. In certain embodiments, administering a therapeutically effective dose of a composition comprising a hairpin-ended DNA molecule as described herein will result an increase in plasma FVIII levels relative to baseline plasma FVIII levels in the subject prior to treatment. In some embodiments, the increase in plasma FVIII levels relative to baseline liver FVIII levels will be at least 5%, 10%, 20%, 30%, 40%, 50%, 100%, 200%, or more.

In some embodiments, a therapeutically effective dose, when administered regularly, results in increased concentration of FVIII in the plasma as compared to baseline levels prior to treatment. In some embodiments, administering a therapeutically effective dose of a composition comprising a hairpin-ended DNA molecule described herein results in the expression of a FVIII protein level (concentration expressed as international unit [IU]) at or above about 0.02 IUIU/ml, about 0.03 IU/ml, about 0.05 IU/ml, about 0.1 IU/ml, about 0.15 IU/ml, about 0.2 IU/ml, about 0.25 IU/ml, about 0.3 IU/ml, about 0.35 IU/ml, about 0.4 IU/ml, about 0.45 IU/ml, about 0.5 IU/ml, about 0.6 IU/ml, about 0.7 IU/ml, about 0.8 IU/ml, about 0.9 IU/ml, about 1.0 IU/ml, about 1.2 IU/ml or about 1.5 IU/ml of the FVIII concentration in the plasma of a treated subject.

In some embodiments, administering a therapeutically effective dose of a composition comprising a hairpin-ended DNA molecule encoding FVIII described herein will result in reduced levels of one or more of markers selected from alanine transaminase (ALT), aspartate transaminase (AST), alkaline phosphatase (ALP) or creatine phosphokinase (CPK).

In some embodiments, a therapeutically effective dose, results in a reduction of usage of exogenous FVIII replacement therapy, in a patient. In some embodiments, administering a therapeutically effective dose of a composition comprising a hairpin-ended DNA molecule described herein results in a reduction of usage of exogenous FVIII replacement therapy by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% as compared to baseline usage of exogenous FVIII replacement therapy in a patient before treatment with the hairpin-ended DNA molecule.

In other exemplary embodiments, a therapeutically effective dose, when administered once or regularly, results in a reduction of partial thromboplastin time in a biological sample. In some embodiments, administering a therapeutically effective dose of a composition comprising a hairpin-ended DNA molecule of this disclosure results in a reduction of partial thromboplastin time in a biological sample (e.g., a blood sample) by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% as compared to baseline partial thromboplastin time before treatment.

In further embodiments, a therapeutically effective dose, when administered regularly, delays the onset of arthropathy in a treated subject. In some embodiments, a therapeutically effective dose, when administered once or regularly, slows the development of arthropathy or reduces the amount of arthropathy in a subject afflicted with Hemophilia A.

### 5.10 Kits

In another aspect, provided herein are kits for expressing human FVIII in vivo, e.g., in a human patient. In some embodiments, a kit provided herein comprises 0.1-500 mg of one or more DNA molecules provided herein. In some embodiments, the kit further comprises a device for administering the dose. In some embodiments, the device is an injection needle.

All patent applications, publications (patents and patent applications, scientific literature, or any other publications), patents, GenBank citations and other database citations, webpage disclosures, commercial catalogs, and other references cited herein are incorporated by reference in their entirety.

### 6. Examples

A number of embodiments have been described. Nevertheless, it will be understood that various examples in this Section (i.e., Section 6) describes specific embodiments herein solely for the purpose of illustration and do not limit the scope as described in the claims or the disclosure. Various modifications can be made without departing from the spirit and scope of what is provided herein.

### 6.1 Example 1—Production of Plasmids Encoding the Vector

The nucleic acid sequences encoding the vectors were designed in silico. Construct 1 encodes for a left inverted repeat, a mini human PGK promoter, a turboluc ORF, SV40 poly (a) and a right inverted repeat (TGCGCGACTCGCTCGCTCACTGAGGCCGGGCGACCAAAGGTCGCCCGACGCCCG GGCTTTGCCCGGGCGGCCTCAGTGAGCGAGCGAGTCGCGCAGAGAGGTTAAAAC CAACTAGACAACTTTGTATATCTAGAGTTGGGGTTGCGCCTTTTCCAAGGCAGCC CTGGGTTTGCGCAGGGACGCGGCTGCTCTGGGCGTGGTTCCGGGAAACGCAGCG GCGCCGACCCTGGGTCTCGCACATTCTTCACGTCCGTTCGCAGCGTCACCCGGAT CTTCGCCGCTACCCTTGTGGGCCCCCCGGCGACGCTTCCTGCTCCGCCCCTAAGT CGGGAAGGTTCCTTGCGGTTCGCGGCGTGCCGGACGTGACAAACGGAAGCCGCA CGTCTCACTAGTACCCTCGCAGACGGACAGCGCCAGGGAGCAATGGCAGCGCGC CGACCGCGATGGGCTGTGGCCAATAGCGGCTGCTCAGCAGGGCGCGCCGAGAGC AGCGGCCGGGAAGGGGCGGTGCGGGAGGCGGGGTGTGGGGCGGTAGTGTGGGC CCTGTTCCTGCCCGCGCGGTGTTCCGCATTCTGCAAGCCTCCGGAGCGCACGTCG GCAGTCGGCTCCCTCGTTGACCGAATCACCGACCTCTCTCCCCAGGCAAGTTTGT ACAAAAAAGCGCGGCCGCGGCAGGCTGCCACCATGGAAACCGATACACTGCTGC TTTGGGTACTTTTGCTGTGGGTGCCCGGCAGTACGGGCGACGCCGCACAACCAGC TAGGAGAGCAGTCCGGAGCCTGGTCCCCTCTTCCgAGGCCGAGGCTGAACGGGG AAAGCTGCCAGGCAAGAAACTCCCCTTAGAGGTTCTCATCGAGCTGGAAGCAAA CGCACGCAAGGCCGGTTGCACTAGGGGCTGCCTCATCTGCCTCAGCAAGATCAA ATGTACAGCAAAGATGAAGAAGTATATTCCTGGCCGCTGTGCAGACTACGGAGG CGACAAAAAAACAGGACAAGCTGGTATAGTTGGCGCCATCGTAGATATCCCCGA GATTAGTGGGTTCAAGGAAATGGAGCCAATGGAGCAATTCATTGCACAGGTTGA TAGATGTGCGGATTGCACTACTGGTTGCCTTAAGGGCTTGGCGAATGTCAAGTGT AGTGATCTTCTGAAAAAGTGGCTACCCGGCCGGTGTGCCACCTTCGCTGACAAAA TACAGAGCGAAGTCGACAATATTAAAGGACTTGCCGGGGACTAATGATAGTGAC ACAAAGTGACGCGTCCTAGAGCTCGCACTGTGCCTTCTAGTTGCCAGCCATCTGT TGTTTGCCCCTCCCCCGTGCCTTCCTTGACCCTGGAAGGTGCCACTCCCACTGTCC TTTCCTAATAAAATGAGGAAATTGCATCGCATTGTCTGAGTAGGTGTCATTCTAT TCTGGGGGGTGGGGTGGGGCAGGACAGCAAGGGGGAGGATTGGGAAGAGAATA GCAGGCATGCTGGGGAGGGCGCTAGCGCAGGAACCCCTTTTAATGGAGTTGGCG AGTCCCTCTCTGCGCGCTCGCTCGCTCACTGAGGCCGGGCGACCAAAGGTCGCCC GACGCCCGGGCTTTGCCCGGGCGGCCTCAGTGAGCGAGCGAGCGCGCAGAGATC GACTC SEQ ID NO:361) with the four restriction sites for nicking endonuclease arranged to lead to ITR overhangs on the 5′ top strand and 3′ bottom strand. Construct 2 encodes for a modified left ITR, a human PGK promoter, a secreted turboluc ORF, SV40 poly (a), a right ITR and a double restriction sites for nicking endonuclease 115 base pairs downstream of the right ITR (TGCGCGACTCGCTCGCTCACTGAGGCCGGGCGACCAAAGGTCGCCCGACGCCCG GGCTTTGCCCGGGCGGCCTCAGTGAGCGAGCGAGTCGCGCAGAGAGGTTAAAAC CAACTAGACAACTTTGTATATCTAGAGTTGGGGTTGCGCCTTTTCCAAGGCAGCC CTGGGTTTGCGCAGGGACGCGGCTGCTCTGGGCGTGGTTCCGGGAAACGCAGCG GCGCCGACCCTGGGTCTCGCACATTCTTCACGTCCGTTCGCAGCGTCACCCGGAT CTTCGCCGCTACCCTTGTGGGCCCCCCGGCGACGCTTCCTGCTCCGCCCCTAAGT CGGGAAGGTTCCTTGCGGTTCGCGGCGTGCCGGACGTGACAAACGGAAGCCGCA CGTCTCACTAGTACCCTCGCAGACGGACAGCGCCAGGGAGCAATGGCAGCGCGC CGACCGCGATGGGCTGTGGCCAATAGCGGCTGCTCAGCAGGGCGCGCCGAGAGC AGCGGCCGGGAAGGGGCGGTGCGGGAGGCGGGGTGTGGGGCGGTAGTGTGGGC CCTGTTCCTGCCCGCGCGGTGTTCCGCATTCTGCAAGCCTCCGGAGCGCACGTCG GCAGTCGGCTCCCTCGTTGACCGAATCACCGACCTCTCTCCCCAGGCAAGTTTGT ACAAAAAAGCGCGGCCGCGGCAGGCTGCCACCATGGAAACCGATACACTGCTGC TTTGGGTACTTTTGCTGTGGGTGCCCGGCAGTACGGGCGACGCCGCACAACCAGC TAGGAGAGCAGTCCGGAGCCTGGTCCCCTCTTCCgAGGCCGAGGCTGAACGGGG AAAGCTGCCAGGCAAGAAACTCCCCTTAGAGGTTCTCATCGAGCTGGAAGCAAA CGCACGCAAGGCCGGTTGCACTAGGGGCTGCCTCATCTGCCTCAGCAAGATCAA ATGTACAGCAAAGATGAAGAAGTATATTCCTGGCCGCTGTGCAGACTACGGAGG CGACAAAAAAACAGGACAAGCTGGTATAGTTGGCGCCATCGTAGATATCCCCGA GATTAGTGGGTTCAAGGAAATGGAGCCAATGGAGCAATTCATTGCACAGGTTGA TAGATGTGCGGATTGCACTACTGGTTGCCTTAAGGGCTTGGCGAATGTCAAGTGT AGTGATCTTCTGAAAAAGTGGCTACCCGGCCGGTGTGCCACCTTCGCTGACAAAA TACAGAGCGAAGTCGACAATATTAAAGGACTTGCCGGGGACTAATGATAGTGAC ACAAAGTGACGCGTCCTAGAGCTCGCACTGTGCCTTCTAGTTGCCAGCCATCTGT TGTTTGCCCCTCCCCCGTGCCTTCCTTGACCCTGGAAGGTGCCACTCCCACTGTCC TTTCCTAATAAAATGAGGAAATTGCATCGCATTGTCTGAGTAGGTGTCATTCTAT TCTGGGGGGTGGGGTGGGGCAGGACAGCAAGGGGGAGGATTGGGAAGAGAATA GCAGGCATGCTGGGGAGGGCGCTAGCGCAGGAACCCCTTTTAATGGAGTTGGCG AGTCCCTCTCTGCGCGCTCGCTCGCTCACTGAGGCCGGGCGACCAAAGGTCGCCC GACGCCCGGGCTTTGCCCGGGCGGCCTCAGTGAGCGAGCGAGCGCGCAGAGATC GACTCCTCGGCCACTTGGAGGGGCCGGGGGGACGACGCAATCTGGAGTGGAAAG AACCCCCGTCTATGCGGCTTAAAGCACGGCCAGGGAATAGTGGATCAAGTGTAC TGACATGTGCCGGAGTCCCTCCATGCCCAGATCGACTCCCTCGAGATATATGGAT CC SEQ ID NO:362). Construct 1 and 2 were synthetized and cloned into a pUC57 backbone (plasmid 1 and plasmid 2, respectively) by a commercial DNA synthesis vendor.

Plasmids 1 & 2 were transformed and then amplified overnight in the NEBstable or MDS-42 strain followed by plasmid isolation using commercial plasmid isolation kit (Nucleobond Xtra Maxi Plus EF (Macherey Nagel)) and dissolved in nuclease free water.

For construct 1: To induce nicks on construct 1, the nicking endonuclease Nt.BstNBI (6.2 U/μg DNA) was added to the isolated construct 1 in 1× Neb3.1 Buffer and incubated at 55° C. for one hour. The reaction mix containing the nicked plasmid was then heated to 90° C. on a thermo shaker for 10 min, in order to dissociate the ITR flanked transgene from the plasmid back bone and the mix was then left to cool to room temperature for 30 min to allow for ITR folding at the single stranded overhangs ends. The reaction mix was then supplemented with both the restriction enzyme Pull and RecBCD Exonuclease V (0.157 U and 0.625 U per μg of nicked plasmid, respectively) as well as adenosine triphosphate (final concentration of 1 mM). The reaction mix was then placed on a shaker at 37° C. for 120 min to allow for the restriction enzyme to cleave the backbone fragment and the exonuclease to digest backbone fragments. The exonuclease generally does not digest linear fragments protected by hairpin ends. Finally, the reaction mix was purified using Takara NucleoSpin Gel and PCR clean-up kit and remaining ITR flanked vector was eluted according to the manufacturer's instructions.

For construct 2: To induce nicks and linearize construct 2, the nicking endonuclease Nt.BstNBI (0.62 U/μg DNA) was added to the isolated construct 2 in 1× Neb3.1 Buffer and incubated at 55° C. for 120 min. The reaction mix containing the nicked construct 2 was then heated to 95° C. on a thermocycler for 3 min in order to dissociate the ITR flanked transgene from the plasmid back bone and subsequently cooled down to 40° C. in the thermocycler with a slope of 0.05° C./s. The reaction mix was then supplemented with Exonuclease V (2.5 U/μg of DNA) as well as adenosine triphosphate (final concentration of 1 mM). The reaction mix was then placed on a shaker at 37° C. for 120 min to allow for the restriction enzyme to cleave the backbone fragment and the exonuclease to digest backbone fragments. The exonuclease generally does not digest linear fragments protected by hairpin ends. Finally, the reaction mix was purified using a Takara NucleoSpin Gel and PCR clean-up kit and remaining ITR flanked vector was eluted according to the manufacturer's instructions.

Nicked, de/renatured and digestion resistant DNA products were visualized by native agarose gel electrophoresis.

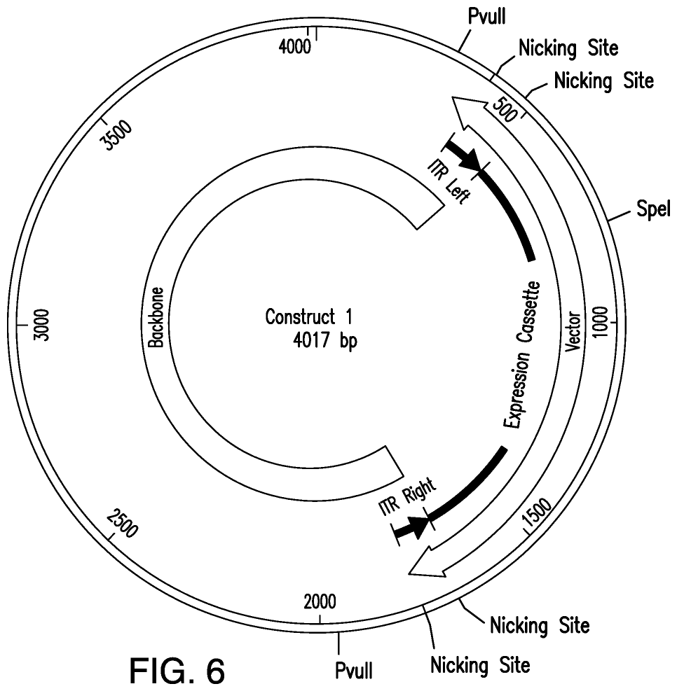

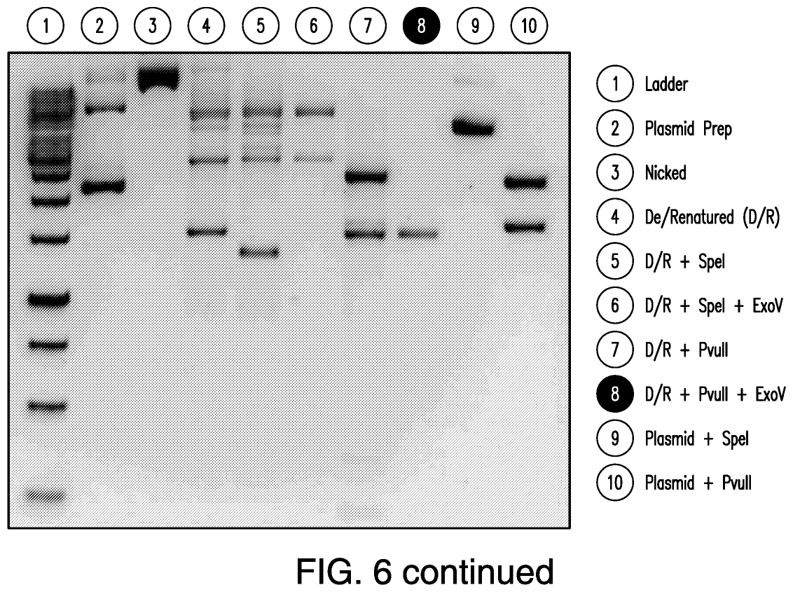

For construct 1, the agarose gel (FIG. 6) shows the nicked plasmid in lane 3, the de/renatured DNA products in lane 4 and the single band of digestion resistant vector in lane 8. To further validate the formation of folded ITRs, the plasmid as well as de/renatured DNA products were digested with the restriction enzyme SpeI, which has a single cut site in the mini PGK promoter region of the vector. As seen in FIG. 6, SpeI digestion cleaves the vector, causing a shift of the lower band in lane 5 and upon treatment with the exonuclease V, the vector fragment is digested while the backbone, which is protected from digestion by the folded anti-sense ITRs, remain. The opposite is true when the de/renatured DNA is digested with the restriction enzyme PvuII, which has two cut sites in the backbone, and following exonuclease V treatment, only the desired product remains (lane 8). Additionally, the construct 1 starting material linearized with SpeI or PvuII, releasing a linear DNA strand in the case of SpeI and two fragments with PvuII. Overall, this demonstrated that a double stranded DNA molecule comprising an expression cassette flanked by two inverted repeats, further each comprising a first and a second restriction site for nicking endonuclease arranged on opposite strands is formed by nicking followed by de/renaturing of the DNA. The formation of such a hairpin ended DNA molecule was further shown to be resistant to exonuclease V digestion.

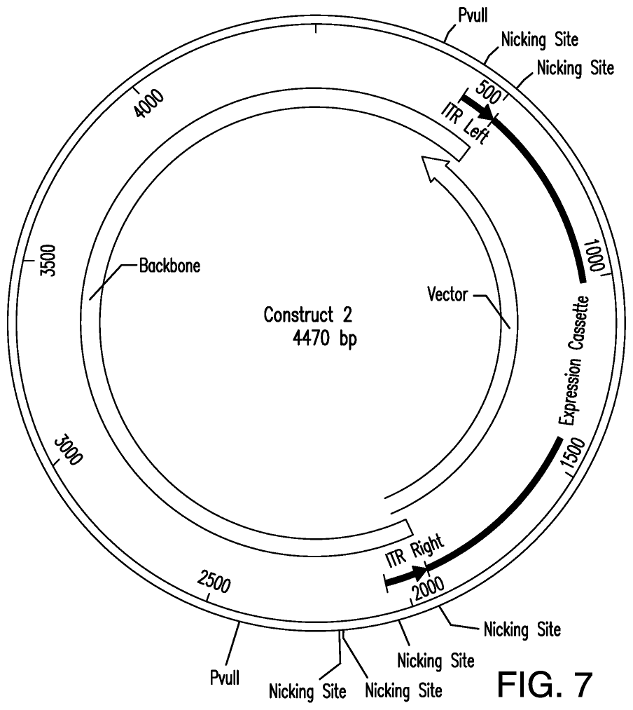

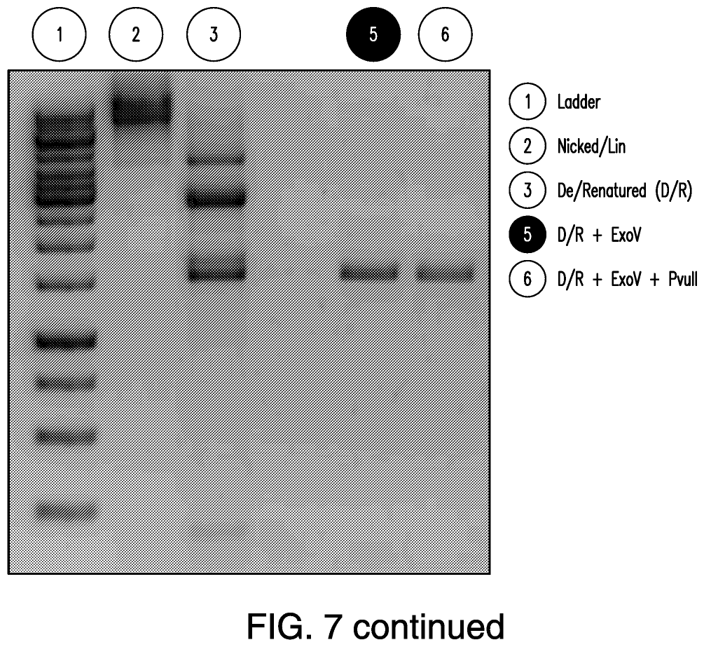

For construct 2, which contains a close proximity double restriction sites for nicking endonuclease downstream of the 3′ ITR, the treatment with the nicking endonuclease BstNBI leads to a linearization of the circular plasmid. As seen in FIG. 7, the agarose gels shows the linearized DNA in lane 2. After de/renaturing, the DNA products are visible in lane 3 and following digestion with RecBCD Exonuclease V a single band of digestion resistant vector is visible in lane 5. To further validate the formation of folded ITRs and the linearization of the plasmid, the re/denatured DNA products were digested with PvuII which has two cut sites in the backbone. Following Exonuclease V digestion, no additional bands were detected. This shows that by incorporating a close proximity double restriction sites for nicking endonuclease upstream of the 5′ ITR or downstream of the 3′ ITR, the backbone can by fragmented simultaneously while the inverted repeat section is nicked, making a further digestion of the backbone by a restriction site obsolete. Thus, the open reading frame of luciferase can easily be replaced with either open reading frame of FVIII by conventional molecular cloning techniques.

### 6.2 Example 2 Repeated De-/Renaturing of DNA Constructs

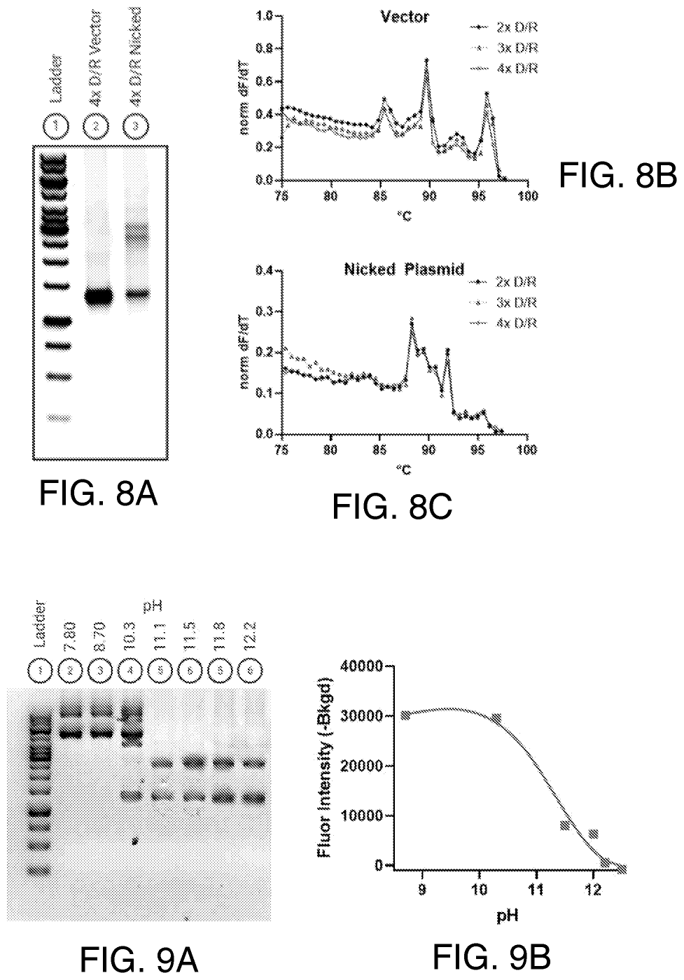

To assess the stability of folding of the DNA constructs, the nicked construct 1 and the corresponding hairpinned vector were subject to multiple cycles of denaturing and annealing. In a first step, 100 ng total DNA was incubated in NEB buffer 3.1 with 1 μM Sybr Green I dye and transferred into a 384 well PCR plate. The plate was sealed and Sybr Green fluorescence was continuously monitored over several 70-98° C. heating cycles performed on a Quantstudio 5 real time PCR machine. Following three heating cycles for the vector (in addition to the heating cycle during production) and 4 cycles for the nicked construct, the samples were visualized by native agarose gel electrophoresis. As seen in FIG. 8A, one band was present for the vector in lane 2 as well as bands corresponding to the vector and back bone were visible in line 3. In addition, the melting curves in FIG. 8B and FIG. 8C show distinct melting fingerprints that remain very similar throughout every cycle.

### 6.3 Example 3 Isothermal Formation of Vector

Aside from dissociating the vector from the nicked plasmid by thermal energy, the possibility of isothermal denaturing using a chemical denaturing agent was tested. Specifically, the capacity of NaOH to act as a denaturing agent to dissociate the nicked construct of example 1 was tested. First the nicked construct (100 ng total DNA) was mixed with 0.2 μM SYBR green I dye in NEB buffer 3.1 that was previously adjusted for corresponding pH values with 1M NaOH. Fluorescence signal was detected at corresponding pH values using a plate reader. As seen in FIG. 9B, as the pH is increased, the background adjusted fluorescence decreases, indicating an increased fraction of denatured DNA. The pH of the denatured DNA was then reverted to pH 7.9 with HCl and the samples were loaded on to an agarose electrophoresis gel. As seen FIG. 9A, at a pH over 10.3 the DNA construct dissociated and formed the expected bands including a band for the corresponding vector. The same band is visible after re/denatured using thermal energy.

### 6.4 Example 4 Transfection of LNPs and Hybridosomes

Lipid nanoparticles were prepared on a Nanoassemblr™ microfluidic system (Precision NanoSystems) according to the manufacturer's instructions. Depending on the desired formulation, an ethanol solution similar to that of the preformed vesicle approach, consisting of an ionizable lipid (e.g. MC3), a zwitterionic lipid (e.g., distearoylphosphatidylcholine (DSPC), dioleoylglycerophosphocholine (DOPC), a component to provide membrane integrity (such as a sterol, e.g., cholesterol) and a conjugated lipid molecule (such as a PEG-lipid, e.g., 1-(monomethoxy-polyethyleneglycol)-2,3-dimyristoylglycerol, with an average PEG molecular weight of 2000 (“PEG-DMG”)) at the appropriate molar ratio (e.g. 40:40:18:2), was prepared at concentrations of 10 mM total lipid. Furthermore, an aqueous DNA solution with a DNA to lipid wt/wt ratio of approximately 14 was prepared in 25 mM acetate buffer at pH 4.0. Depending on the total volume of production 1 and 3 ml syringes where used to create the inlet stream with a total flow rate of 12 ml/min. For each formulation the aqueous DNA solution was mixed with the ethanol-lipid solution with a flow rate ratio of 3:1 (Aq:Et) at room temperature. The product was then dialyzed against PBS to remove the residual ethanol as well as to raise the pH to 7.4.

For exosome production, cells were grown in stirred bioreactors in perfusion mode and exosome isolation was performed by tangential flow filtration followed by Captocore 700 liquid chromatography as described in Nordin et al Methods in Molecular Biology, vol 1953. Humana Press, New York, NY (2019), which is herein incorporated in its entirety by reference.

Human embryonic kidney cells (HEK-293T) were cultured in DMEM supplemented with 10% FCS. The cells were grown at 37° C. in a 5% CO₂-humified incubator. For testing the Luciferase expression, cells (2×10⁴/well) were seeded in a 96-well plate and transfected for 48 h with 100 ng, 10 ng and Ing (0.37 pmol, 0.031 pmol, 0.001 pmol and 0.001 pmol) of DNA vector coding for Turboluc. Transfection was mediated using Hybridosomes generated by fusing exosomes with lipid nanoparticles as outlined in U.S. Ser. No. 15/112,180. As a comparison, cells additionally were transfected with lipid nanoparticles.

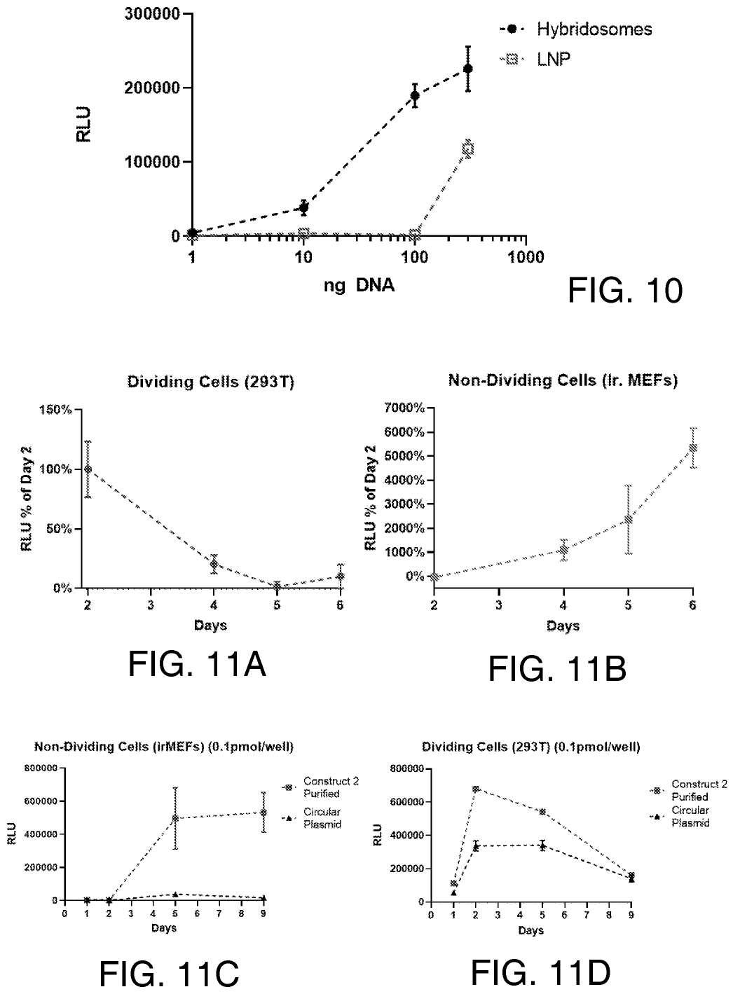

Luciferase expression level was determined 48 h after transfection using the Gluc Glow Assay kit from NanoLight Technology. 150 μl of medium in wells containing transfected cells was removed (50 μl of medium left in wells) and 50 μl Coelenterazine in cell lysis buffer (NanoLight Technology) was added at a final concentration of 50 μM Coelenterazine. The cells then were incubated at room temperature for 5 min. while being shaken in the dark. The cell lysis containing the luciferase substrate was transferred into a white 96-well plate (BRAND). Luciferase activity was determined by measuring the luminescence using a SynergyMX plate reader (BioTek) and shown in FIG. 10.

### 6.5 Example 5 Expression in Dividing and Non-Dividing Cells

Constructs were generated to include an open reading frame encoding the Turboluc reporter gene into the expression cassette flanked by two ITRs. Expression of Turboluc from the vectors over time was determined based on Luciferase activity in each cell culture, confirming that the Luciferase activity resulted from gene expression from the vector.

In detail, human embryonic kidney cells (HEK-293T) were cultured in DMEM (10% FCS, 1% pen/strep) and 2 mM stable Glutamine. Irradiated mouse embryonic fibroblasts (C57BL/6, Thermo Fisher) were cultured in DMEM (10% FCS, 1% pen/strep), 2 mM stable Glutamine and 1 mM Sodium Pyruvate. The cells were grown at 37° C. in a 5% CO₂-humified incubator. Cells (6×10+/well) were seeded in a 96-well plate coated with 0.1% Gelatin solution and transfected with 100 ng (0.13 pmol) of folded ITR DNA vector 1 of example 1. Transfections were done using JetPrime (PolyPlus) transfection reagent according to the manufacturer's instructions. Culture medium was changed 3 days post transfection. Luciferase expression level was determined at different days after transfection, starting with day 1 post transfection using the Gluc Glow Assay kit (NanoLight Technology). The medium in wells containing transfected cells was replaced with phosphate-buffered saline (50 μl) and 50 μl Coelenterazine in cell lysis buffer (NanoLight Technology) was added at a final concentration of 50 μM Coelenterazine. The cells then were incubated at room temperature for 5 min, while being shaken in the dark. The cell lysis containing the luciferase substrate was transferred into a white 96-well plate. For construct 2, which encodes for a secreted Turboluc, a 50 μl sample supernatant was removed at on different days and the complete medium was exchanged. The supernatant sample was incubated for 2 minutes with Coelenterazine at a final concentration of 50 μM. Luciferase activity was determined by measuring the luminescence using a SynergyMX plate reader (BioTek). For the analysis of background, bioluminescence from untreated cells was measured following the protocol mentioned above. As seen in FIG. 11A and FIG. 11B, for construct 1, expressing non-secreted Turboluc, luciferase activity peaks in dividing cells on day 2, while in non-dividing cells the expression continues to increase. As seen in FIG. 11C and FIG. 11D, for construct 2 encoding secreted Turboluc, luciferase activity peaks in dividing cells on day 2, while in non-dividing cells the expression increases and then remains stable over 9 days. As a direct comparison, equimolar amounts of full circular plasmids encoding construct 2 were also transfected and as seen in FIG. 11C and FIG. 11D, generally a lower luciferase activity was recorded, indicating improved nuclear delivery of the purified construct 2 with folded ITRs.

### 6.6 Example 6 Long Term Expression of Ligated and Non-Ligated Constructs

Construct 3 is a construct with the four restriction sites for nicking endonuclease arranged to lead to ITR overhangs on the 5′ top strand and 3′ bottom strand. Construct 3 encodes for a modified left ITR, a truncated SFFV promoter, a mouse kappa IgG leader, a turboluc ORF, bGH poly(A) signal (a), a right ITR and a double restriction sites for nicking endonuclease 115 base pairs downstream of the right ITR (TGCGCGACTCGCTCGCTCACTGAGGCCGGGCGACCAAAGGTCGCCCGACGCCCG GGCTTTGCCCGGGCGGCCTCAGTGAGCGAGCGAGTCGCGCAGAGAGGTTAAAAC CAACTAGACAACTTTGTATATCTAGAGTAACGCCATTTTGCAAGGCATGGAAAA ATACCAAACCAAGAATAGAGAAGTTCAGATCAAGGGCGGGTACATGAAAATAG CTAACGTTGGGCCAAACAGGATATCTGCGGTGAGCAGTTTCGGCCCCGGCCCGG GGCCAAGAACAGATGGTCACCGCAGTTTCGGCCCCGGCCCGAGGCCAAGAACAG ATGGTCCCCAGATATGGCCCAACCCTCAGCAGTTTCTTAAGACCCATCAGATGTT TCCAGGCTCCCCCAAGGACCTGAAATGACCCTGCGCCTTATTTGAATTAACCAAT CAGCCTGCTTCTCGCTTCTGTTCGCGCGCTTCTGCTTCCCGAGCTCTATAAAAGAG CTCACAACCCCTCACTCGGCGCGCCAGTCCTCCGATTGACTGAGTGCGGCCGCGG CAGGCTGCCACCATGGAAACCGATACACTGCTGCTTTGGGTACTTTTGCTGTGGG TGCCCGGCAGTACGGGCGACGCCGCACAACCAGCTAGGAGAGCAGTCCGGAGCC TGGTCCCCTCTTCCGAGGCCGAGGCTGAACGGGGAAAGCTGCCAGGCAAGAAAC TCCCCTTAGAGGTTCTCATCGAGCTGGAAGCAAACGCACGCAAGGCCGGTTGCA CTAGGGGCTGCCTCATCTGCCTCAGCAAGATCAAATGTACAGCAAAGATGAAGA AGTATATTCCTGGCCGCTGTGCAGACTACGGAGGCGACAAAAAAACAGGACAAG CTGGTATAGTTGGCGCCATCGTAGATATCCCCGAGATTAGTGGGTTCAAGGAAAT GGAGCCAATGGAGCAATTCATTGCACAGGTTGATAGATGTGCGGATTGCACTAC TGGTTGCCTTAAGGGCTTGGCGAATGTCAAGTGTAGTGATCTTCTGAAAAAGTGG CTACCCGGCCGGTGTGCCACCTTCGCTGACAAAATACAGAGCGAAGTCGACAAT ATTAAAGGACTTGCCGGGGACTAATGATAGTGACACAAAGTGACGCGTCCTAGA GCTCGCACTGTGCCTTCTAGTTGCCAGCCATCTGTTGTTTGCCCCTCCCCCGTGCC TTCCTTGACCCTGGAAGGTGCCACTCCCACTGTCCTTTCCTAATAAAATGAGGAA ATTGCATCGCATTGTCTGAGTAGGTGTCATTCTATTCTGGGGGGTGGGGTGGGGC AGGACAGCAAGGGGGAGGATTGGGAAGAGAATAGCAGGCATGCTGGGGAGGGC GCTAGCGCAGGAACCCCTTTTAATGGAGTTGGCGAGTCCCTCTCTGCGCGCTCGC TCGCTCACTGAGGCCGGGCGACCAAAGGTCGCCCGACGCCCGGGCTTTGCCCGG GCGGCCTCAGTGAGCGAGCGAGCGCGCAGAGATCGACTCCTCGGCCACTTGGAG GGGCCGGGGGGACGACGCAATCTGGAGTGGAAAGAACCCCCGTCTATGCGGCTT AAAGCACGGCCAGGGAATAGTGGATCAAGTGTACTGACATGTGCCGGAGTCCCT CCATGCCCAGATCGACTCCCTCGAG SEQ ID NO:335). Construct 3 was produced by the same method as for construct 2 in Example 1. Following purification by a Gel and PCR clean-up kit, the purified vector was mixed with T4 ligase and T4 ligation buffer according to the manufacturer instructions and was incubated at 25° C. for 2 h. To confirm complete ligation, the pH of samples of the ligated construct was adjusted with 100 mM Tris-HCl (pH 8.8), T5 exonuclease was added to the mix which was then incubated for 1 h at 37° C. Ligation was further confirmed by dilution of a sample of the ligated construct in water followed by the addition of formamide and loading dye. This mix was incubated at 65° C. for 5 mins and then kept on ice until loading in onto the agarose gel. After the ligation step, the ligated construct was re-purified using a Gel and PCR clean-up kit.

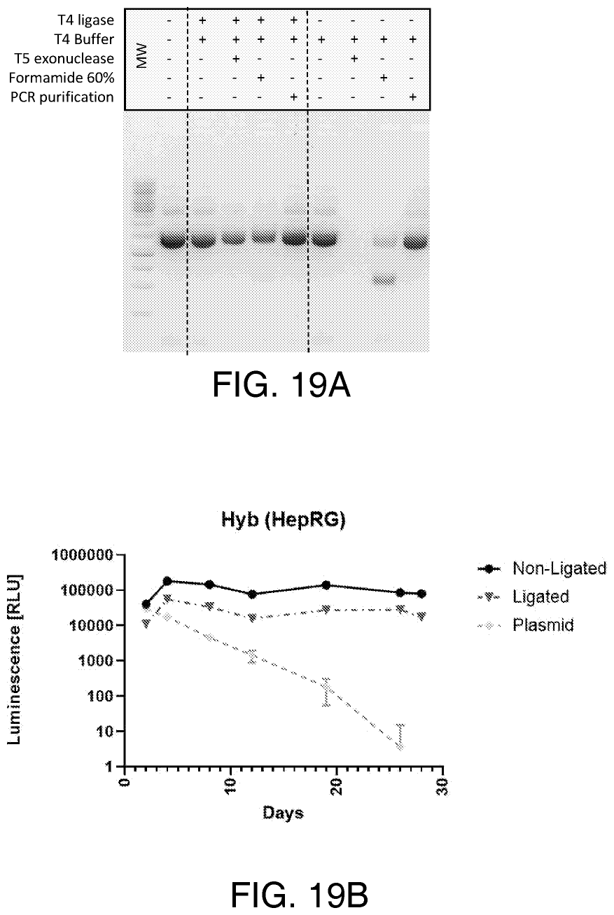

To confirm ligation, results were analyzed using an agarose gel. The agarose gel (FIG. 19A) shows the purified construct in lane 2, the ligase & construct mix after ligation in lane 3, the T5 exonuclease treated, previously ligated construct in lane 4, the formamide denatured, previously ligated construct in lane 5 and the resulting re-purified ligated construct in lane 6. The second half of the agarose gel shows the non-ligated construct in ligation buffer (lane 7), T5 exonuclease treated (lane 8), formamide denatured (lane 9) and re-purified (lane 10).

The purified ligated construct, non-ligated construct and circular parent plasmid were subsequently loaded into LNPs, as well as Hybridosomes as outlined in Example 4. Expression of secreted turboluc following transfection of non-dividing HepRG cells with hybridosome encapsulating ligated construct, non-ligated construct or parent circular plasmid was monitored over time (FIG. 19B). While the expression from the transfection with the circular plasmid quickly diminished, the ligated construct and the non-ligated construct remained stable over time. Surprisingly, expression from the non-ligated construct was observed at levels that are higher than the levels seen in transfection with the ligated construct.

### 6.7 Example 7 Long Term Expression after Transfection with LNPs and Hybridosomes

Constructs were generated to include an open reading frame encoding the Turboluc reporter gene into the expression cassette flanked by two ITRs. Expression of secreted Turboluc from the vectors over time was determined based on luciferase activity.

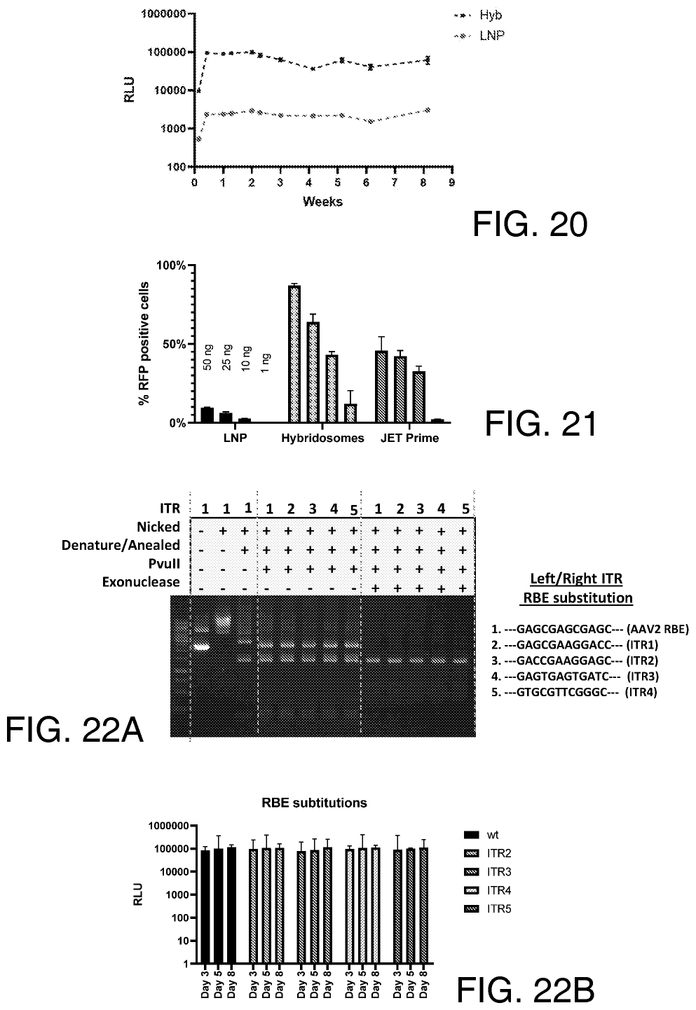

In detail, differentiated non-dividing HepRG cells were maintained in HepaRG™ Maintenance/Metabolism media and transfected with LNPs and Hybridosomes, previously prepared as outlined in example 7. To analyze duration of expression luciferase expression level was determined at different time points for non-dividing cells (FIG. 20) as described in example 7. Luciferase activity was determined by measuring the luminescence using a SynergyMX plate reader (BioTek). For the analysis of background, bioluminescence from untreated cells was measured following the protocol described in Example 1 above. As seen in FIG. 20, for non-dividing cells transfected with a construct encoding secreted Turboluc, luciferase activity remains stable over two months.

### 6.8 Example 8 Transfection with Cre Encoding Hairpin Ended DNA

A construct was designed in silico encoding a left ITR, a EF1a promoter modified to remove nicking sites, a Cre-recombinase ORF, bGH poly(A) signal (a), a right ITR and a double restriction sites for nicking endonuclease 115 base pairs downstream of the right ITR. The construct was ordered to be synthesized and cloned into a pUC mini backbone. The hairpin-ended DNA was produced from circular plasmids as in example 1 and the purified DNA molecules were subsequently loaded into LNPs, as well as Hybridosomes as outlined in Example 4. The percentage of cells transfected was monitored using a monoclonally expanded HEK293-loxP-GFP-RFP (GenTarget) stable cell line. Upon successful transfection Cre recombinase protein is expressed in the cells, enters the nuclease and excises the floxed GFP-stop, to switch the cells from GFP positive to RFP positive. The vector transfected with JetPrime acted as a positive control. The HEK293-loxP-GFP-RFP cells were plated in 96 well plates and transfected with four different doses of DNA. After 72 h the cells were trypsinated and analyzed by flow cytometry for RFP expression. As seen in FIG. 21, at the highest dose, Hybridosomes successfully transfected >85% of the cells while LNPs and positive control JetPrime showed limited transfection efficiency of the cell population.

### 6.9 Example 9 RABS Substitutions

In order to investigate the effect of an AAV RBE on the process and transfection efficiency, de novo DNA backbones encoding a right ITR, a pUC ori, Ampicillin resistance and a left ITR were designed and synthesized as well as cloned into a kanamycin resistance vector by a commercial vendor. The vectors were digested and the synthesized fragment excised from the kanamycin backbone and isolated by gel extraction. The synthesized backbone fragment was then purified and the expression cassette of example 7 encoding for secreted turboluc was ligated with synthesized backbones, to circularize the plasmids. Five backbones encoding the left and right ITRs shown below between the expression cassette were produced and amplified in Stabl3 bacteria. The plasmids were then nicked with Nt.BstNBI and hairpin ended DNA was produced as described in Example 1. The production of the hairpin ended DNA was confirmed by running an agarose gel as shown in FIG. 22. The gel shows no pronounced differences in products nor yield. Next, HepRG cells were transfected with 50 ng of the purified hairpin ended DNAs using JetPrime according to the manufacturer instructions and levels of secreted turboluc were monitored on several days as described in Example 7.

| | Left ITR | Right ITR |
|---|---|---|
| Plasmid | GAGTCCCTC | TTGGCGAGTC |
| 1 | TCTGCGCGC | CCTCTCTGCG |
| AAV2 | TCGCTCGCT | CGCTCGCTCG |
| RBE | CACTGAGGC | CTCACTGAGG |
| | CGGGCGACC | CCGGGCGACC |
| | AAAGGTCGC | AAAGGTCGCC |
| | CCGACGCCC | CGACGCCCGG |
| | GGGCTTTGC | GCTTTGCCCG |
| | CCGGGCGGC | GGCGGCCTCA |
| | CTCAGTGAG | GTGAGCGAGC |
| | CGAGCGAGC | GAGCGCGCAG |
| | GCGCAGAGA | AGAGGGACTC |
| | GGGACTCGC | (SEQ ID |
| | CAA | NO: 369) |
| | (SEQ ID | |
| | NO: 368) | |
| Plasmid | GAGTCCCTC | TTGGCGAGTC |
| 2 | TCTGCGCGG | CCTCTCTGCG |
| ITR1 | TCCTTCGCT | CGGTCCTTCG |
| | CACTGAGGC | CTCACTGAGG |
| | CGGGCGACC | CCGGGCGACC |
| | AAAGGTCGC | AAAGGTCGCC |
| | CCGACGCCC | CGACGCCCGG |
| | GGGCTTTGC | GCTTTGCCCG |
| | CCGGGCGGC | GGCGGCCTCA |
| | CTCAGTGAG | GTGAGCGAAG |
| | CGAAGGACC | GACCGCGCAG |
| | GCGCAGAGA | AGAGGGACTC |
| | GGGACTCGC | (SEQ ID |
| | CAA | NO: 371) |
| | (SEQ ID | |
| | NO: 370) | |
| Plasmid | GAGTCCCTC | TTGGCGAGTC |
| 3 | TCTGCGCGC | CCTCTCTGCG |
| ITR2 | TCCTTCGGT | CGCTCCTTCG |
| | CACTGAGGC | GTCACTGAGG |
| | CGGGCGACC | CCGGGCGACC |
| | AAAGGTCGC | AAAGGTCGCC |
| | CCGACGCCC | CGACGCCCGG |
| | GGGCTTTGC | GCTTTGCCCG |
| | CCGGGCGGC | GGCGGCCTCA |
| | CTCAGTGAC | GTGACCGAAG |
| | CGAAGGAGC | GAGCGCGCAG |
| | GCGCAGAGA | AGAGGGACTC |
| | GGGACTCGC | (SEQ ID |
| | CAA | NO: 373) |
| | (SEQ ID | |
| | NO: 372) | |
| Plasmid | GAGTCCCTC | TTGGCGAGTC |
| 4 | TCTGCGCGA | CCTCTCTGCG |
| ITR3 | TCACTCACT | CGATCACTCA |
| | CACTGAGGC | CTCACTGAGG |
| | CGGGCGACC | CCGGGCGACC |
| | AAAGGTCGC | AAAGGTCGCC |
| | CCGACGCCC | CGACGCCCGG |
| | GGGCTTTGC | GCTTTGCCCG |
| | CCGGGCGGC | GGCGGCCTCA |
| | CTCAGTGAG | GTGAGTGAGT |
| | TGAGTGATC | GATCGCGCAG |
| | GCGCAGAGA | AGAGGGACTC |
| | GGGACTCGC | (SEQ ID |
| | CAA | NO: 375) |
| | (SEQ ID | |
| | NO: 374) | |
| Plasmid | GAGTCCCTC | TTGGCGAGTC |
| 5 | TCTGCGCGC | CCTCTCTGCG |
| ITR4 | CCGAACGCA | CGCCCGAACG |
| | CACTGAGGC | CACACTGAGG |
| | CGGGCGACC | CCGGGCGACC |
| | AAAGGTCGC | AAAGGTCGCC |
| | CCGACGCCC | CGACGCCCGG |
| | GGGCTTTGC | GCTTTGCCCG |
| | CCGGGCGGC | GGCGGCCTCA |
| | CTCAGTGTG | GTGTGCGTTC |
| | CGTTCGGGC | GGGCGCGCAG |
| | GCGCAGAGA | AGAGGGACTC |
| | GGGACTCGC | (SEQ ID |
| | CAA | NO: 377) |
| | (SEQ ID | |
| | NO: 376) | |

### 6.10 Example 10 Production of Hairpin Ended DNA with Double Nicking Sites

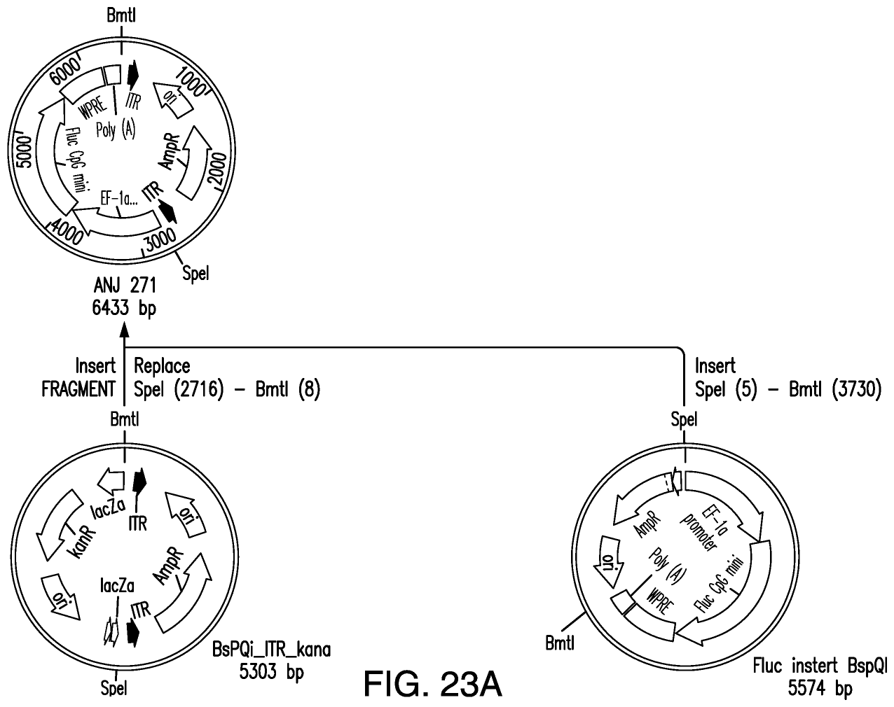

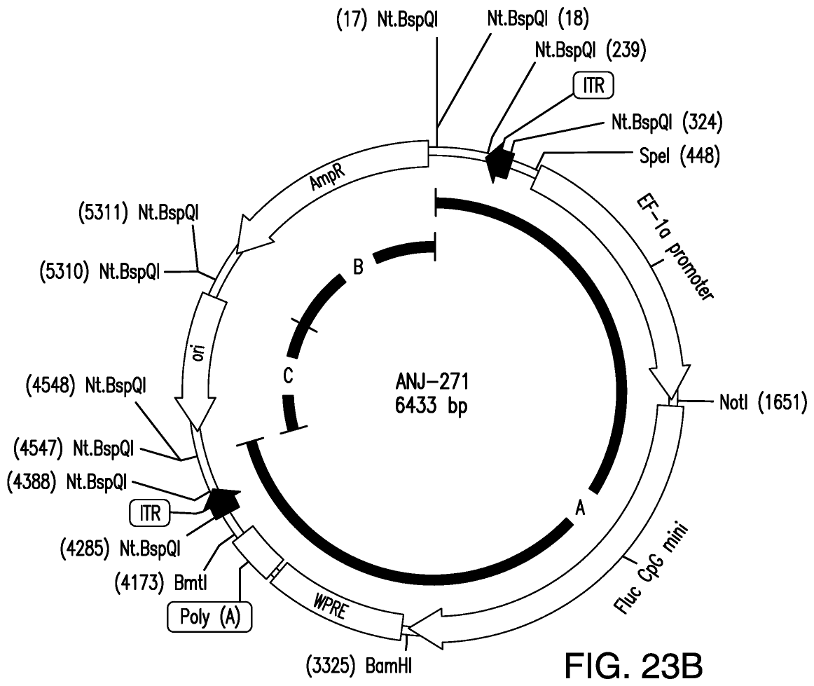

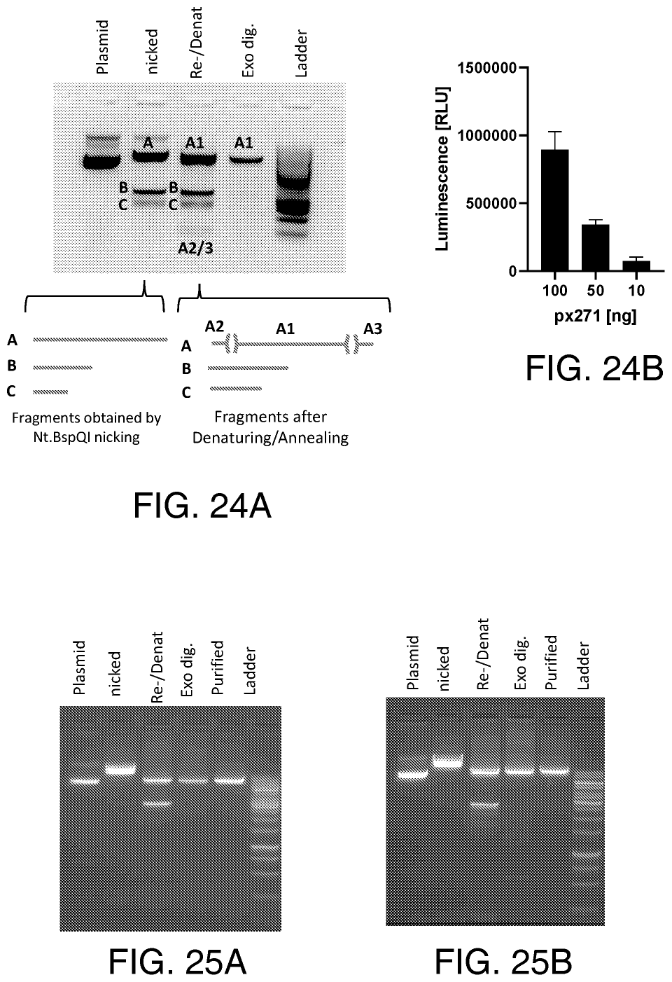

A DNA fragment was de novo designed to contain double nicking sites (i) between the right ITR, the pUC ori, (ii) between the pUC ori and the ampicillin resistance, as well as (iii) between the ampicillin resistance and the left ITR and synthesized as well as cloned into a kanamycin resistance vector by a commercial vendor. Analogous to Example 10, the ITR and backbone fragment was then excised, and gel purified to be ligated with a firefly luciferase expression cassette as depicted in FIG. 23B (corresponding sequence below). The plasmid was amplified, 0.5 mg of plasmid nicked with Nt.BspQI enzyme NEB (40 μl) at 500 ng/μl in nicking buffer and incubated for 1 h at 37 deg. The nicked plasmid was then denatured by addition of 33 mM of NaOH followed by annealing with 41.6 mM Tris pH 7.2. Then the annealed DNA fragments were digested with exonuclease V. Samples of each step were loaded onto an agarose gel and as seen in FIG. 24A, after nicking, the plasmid is split into the 3 expected fragments (A, B & C), all of which have double stranded ends. Upon denaturing and annealing, the A band is further fragmented into a slightly lower A1 band as well as a new faint band labeled A2/A3. This corresponds to the release of the overhangs from the product as depicted in FIG. 24A. After digestion only band A1 is resistant to digestion. The digestion resistant hairpin ended DNA was then purified as described in Example 1 and HEK293T cells were transfected using JetPrime according to the manufacturer instructions. After 48 hours, luciferase expression was recorded using the Steady-Glo luminescence assay on a SynergyMX plate reader (BioTek) and shown in FIG. 24B.

SEQ ID NO: 337
(GTTCGAGATCGCTCTTCAGAAGAGCGCGG
AGTCACATTCGCGACTCGCAATGCATTGCA
GGGCGAGGGCGAGGGCCGCCCCTACGAGGG
CCACAACACCGTGAAGCTGAAGGTGACCAA
GGGCGGCCCCCTGCCCTTCGCCTGGGACAT
CCTGTCCCCCCAGTTCCAGTACGGCTCCAA
GGTGTACGTGAAGCACCCCGCCGACATCCC
AGCTCGGTAGCCACCTGACGTCGCTCTTCG
CGCTCGCTCGCTCACTGAGGCCGGGCGACC
AAAGGTCGCCCGACGCCCGGGCTTTGCCCG
GGCGGCCTCAGTGAGCGAGCGAGCGCGAAG
AGCGGGAGTGAGTGGCCAAAAAGTACCCGG
CCAGAAAGGAACCGTAAAACCAACTAGACA
GCCGGTGACTTTGTTCCCCAGCTCGGTTCA
TTCTCAAGCCTCAGACAGGAGAACCGGGAC
TAGTATATGAGCTCCGGTGCCCGTCAGTGG
GCAGAGCGCACATCGCCCACAGTCCCCGAG
AAGTTGGGGGGAGGGGTCGGCAATTGAACC
GGTGCCTAGAGAAGGTGGCGCGGGGTAAAC
TGGGAAAGTGATGTCGTGTACTGGCTCCGC
CTTTTTCCCGAGGGTGGGGGAGAACCGTAT
ATAAGTGCAGTAGTCGCCGTGAACGTTCTT
TTTCGCAACGGGTTTGCCGCCAGAACACAG
GTAAGTGCCGTGTGTGGTTCCCGCGGGCCT
GGCCTCTTTACGGGTTATGGCCCTTGCGTG
CCTTGAATTACTTCCACGCCCCTGGCTGCA
GTACGTGATTCTTGATCCCGAGCTTCGGGT
TGGAAGTGGGTGGGAGAGTTCGAGGCCTTG
CGCTTAAGGAGCCCCTTCGCCTCGTGCTTG
AGTTGAGGCCTGGCCTGGGCGCTGGGGCCG
CCGCGTGCGAATCTGGTGGCACCTTCGCGC
CTGTCTCGCTGCTTTCGATAAGTCTCTAGC
CATTTAAAATTTTTGATGACCTGCTGCGAC
GCTTTTTTTCTGGCAAGATAGTCTTGTAAA
TGCGGGCCAAGATCGATCTGCACACTGGTA
TTTCGGTTTTTGGGGCCGCGGGCGGCGACG
GGGCCCGTGCGTCCCAGCGCACATGTTCGG
CGAGGCGGGGCCTGCGAGCGCGGCCACCGA
GAATCGGACGGGGGTAGTCTCAAGCTGGCC
GGCCTGCTCTGGTGCCTGGCCTCGCGCCGC
CGTGTATCGCCCCGCCCTGGGCGGCAAGGC
TGGCCCGGTCGGCACCAGTTGCGTGAGCGG
AAAGATGGCCGCTTCCCGGCCCTGCTGCAG
GGAGCTCAAAATGGAGGACGCGGCGCTCGG
GAGAGCGGGCGGGTGAGACACCCACACAAA
GGAAAAGGGCCTTTCCGTCCTCAGCCGTCG
CTTCATGTGACACCACGGAGTACCGGGCGC
CGTCCAGGCACCTCGATTAGTTCTCGATCG
AGCTTTTGGAGTACGTCGTCTTTAGGTTGG
GGGGAGGGGTTTTATGCGATGGAGTTTCCC
CACACTGAGTGGGTGGAGACTGAAGTTAGG
CCAGCTTGGCACTTGATGTAATTCTCCTTG
GAATTTGCCCTTTTTGAGTTTGGATCTTGG
TTCATTCTCAAGCCTCAGACAGTGGTTCAA
AGTTTTTTTCTTCCATTTCAGGTGTCGTGA
GCGGCCGCGGCAGGCTGCCACCATGGAGGA
TGCCAAGAATATTAAGAAAGGCCCTGCCCC
ATTCTACCCTCTGGAAGATGGCACTGCTGG
TGAGCAACTGCACAAGGCCATGAAGAGGTA
TGCCCTGGTCCCTGGCACAATTGCCTTCAC
TGATGCTCACATTGAGGTGGACATCACCTA
TGCTGAATACTTTGAGATGTCTGTGAGGCT
GGCAGAAGCCATGAAAAGATATGGACTGAA
CACCAACCACAGGATTGTGGTGTGCTCTGA
GAACTCTCTCCAGTTCTTCATGCCTGTGTT
GGGAGCCCTGTTCATTGGAGTGGCTGTGGC
CCCTGCCAATGACATCTACAATGAGAGAGA
GCTCCTGAACAGCATGGGCATCAGCCAGCC
AACTGTGGTCTTTGTGAGCAAGAAGGGCCT
GCAAAAGATCCTGAATGTGCAGAAGAAGCT
GCCCATCATCCAGAAGATCATCATCATGGA
CAGCAAGACTGACTACCAGGGCTTCCAGAG
CATGTATACCTTTGTGACCAGCCACCTCCC
CCCTGGCTTCAATGAGTATGACTTTGTGCC
TGAGAGCTTTGACAGGGACAAGACAATTGC
TCTGATTATGAACAGCTCTGGCTCCACTGG
ACTGCCCAAAGGTGTGGCTCTGCCCCACAG
AACTGCTTGTGTGAGATTCAGCCATGCCAG
AGACCCCATCTTTGGCAACCAGATCATCCC
TGACACTGCCATCCTGTCTGTGGTTCCATT
CCATCATGGCTTTGGCATGTTCACAACACT
GGGGTACCTGATCTGTGGCTTCAGAGTGGT
GCTGATGTATAGGTTTGAGGAGGAGCTGTT
TCTGAGGAGCTTGCAAGACTACAAGATCCA
GTCTGCCCTGCTGGTGCCCACTCTGTTCAG
CTTCTTTGCCAAGAGCACCCTCATTGACAA
GTATGACCTGAGCAACCTGCATGAGATTGC
CTCTGGAGGAGCACCCCTGAGCAAGGAGGT
GGGTGAGGCTGTGGCAAAGAGGTTCCATCT
CCCAGGAATCAGACAGGGCTATGGCCTGAC
TGAGACCACCTCTGCCATCCTCATCACCCC
TGAAGGAGATGACAAGCCTGGTGCTGTGGG
CAAGGTGGTTCCCTTTTTTGAGGCCAAGGT
GGTGGACCTGGACACTGGCAAGACCCTGGG
AGTGAACCAGAGGGGTGAGCTGTGTGTGAG
GGGTCCCATGATCATGTCTGGCTATGTGAA
CAACCCTGAGGCCACCAATGCCCTGATTGA
CAAGGATGGCTGGCTGCACTCTGGTGATAT
TGCCTACTGGGATGAGGATGAGCACTTTTT
CATTGTGGACAGGCTGAAGAGTCTCATCAA
GTACAAAGGCTACCAAGTGGCACCTGCTGA
GCTTGAGAGCATCCTGCTCCAGCACCCCAA
CATCTTTGATGCTGGTGTGGCTGGCCTGCC
TGATGATGATGCTGGAGAGCTGCCTGCTGC
TGTTGTGGTTCTGGAGCATGGAAAGACCAT
GACTGAGAAGGAGATTGTGGACTATGTGGC
CAGTCAGGTGACCACTGCCAAGAAGCTGAG
GGGAGGTGTGGTGTTTGTGGATGAGGTGCC
AAAGGGTCTGACTGGCAAGCTGGATGCCAG
AAAGATCAGAGAGATCCTGATCAAGGCCAA
GAAGGGAGGAAAGATTGCAGTTTAAGGATC
CTGACACAAAGTGACGCGTAATCAACCTCT
GGATTACAAAATTTGTGAAAGATTGACTGG
TATTCTTAACTATGTTGCTCCTTTTACGCT
ATGTGGATACGCTGCTTTAATGCCTTTGTA
TCATGCTATTGCTTCCCGTATGGCTTTCAT
TTTCTCCTCCTTGTATAAATCCTGGTTGCT
GTCTCTTTATGAGGAGTTGTGGCCCGTTGT
CAGGCAACGTGGCGTGGTGTGCACTGTGTT
TGCTGACGCAACCCCCACTGGTTGGGGCTT
TGCCACCACCTGTCAGCTCCTTTCCGGGAC
TTTCGCTTTCCCCCTCCCTATTGCCACGGC
GGAACTCATCGCCGCCTGCCTTGCCCGCTG
CTGGACAGGGGCTCGGCTGTTGGGCACTGA
CAATTCCGTGGTGTTGTCGGGGAAATCATC
GTCCTTTCCTTGGCTGCTCGCCTGTGTTGC
CACCTGGATTCTGCGCGGGACGTCCTTCTG
CTACGTCCCTTCGGCCCTCAATCCAGCGGA
CCTTCCTTCCCGCGGCCTGCTGCCGGCTCT
GCGGCCTCTTCCGCGTCTTCGCCTTCGCCC
TCAGACGAGTCGGATCTCCCTTTGGGCCGC
CTCCCCGCACGCGTCCTAGAGCTCGCACTG
TGCCTTCTAGTTGCCAGCCATCTGTTGTTT
GCCCCTCCCCCGTGCCTTCCTTGACCCTGG
AAGGTGCCACTCCCACTGTCCTTTCCTAAT
AAAATGAGGAAATTGCATCGCATTGTCTGA
GTAGGTGTCATTCTATTCTGGGGGGGGGGG
GGGCAGGACAGCAAGGGGGAGGATTGGGAA
GAGAATAGCAGGCATGCTGGGGAGGGCGCT
AGCGCAGGGGGACCCCAAGTGACGATTGAA
CCCCGATTCAGGTACCCATTGGAAATTTTA
ATGGAGTAACATAACTGCTGGAGTAGATGA
AGAACCTCTTGGCCACTCCGCTCCCGAAGA
GCGCACTCGCTCGCTCGGTGGGGCCTGGCG
ACCAAAGGTCGCCAGACGGACGTGCTTTGC
ACGTCCGGCCCCACCGAGCGAGCGAGTGCG
CTCTTCGGGCCACTTGGAGGGGCCGGGGGG
ACGACGCAATCTGGAGTGGAAAGAACCCCC
GTCTATGCGGCTTAAAGCACGGCCAGGGAA
TAGTGGATCAAGTGTACTGACATGTGCCGG
CAATGCATTGCGTCCCGAGTCACATTCGCG
ACTCTCGACGCTCTTCAGAAGAGCAGCTTA
GCTTCAATAGCTCAATGATATCGGAAAGAA
CATGTGAGCAAAAGGCCAGCAAAAGGCCAG
GAACCGTAAAAAGGCCGCGTTGCTGGCGTT
TTTCCATAGGCTCCGCCCCCCTGACGAGCA
TCACAAAAATCGACGCTCAAGTCAGAGGTG
GCGAAACCCGACAGGACTATAAAGATACCA
GGCGTTTCCCCCTGGAAGCTCCCTCGTGCG
CTCTCCTGTTCCGACCCTGCCGCTTACCGG
ATACCTGTCCGCCTTTCTCCCTTCGGGAAG
CGTGGCGCTTTCTCATAGCTCACGCTGTAG
GTATCTCAGTTCGGTGTAGGTCGTTCGCTC
CAAGCTGGGCTGTGTGCACGAACCCCCCGT
TCAGCCCGACCGCTGCGCCTTATCCGGTAA
CTATCGTCTTGAGTCCAACCCGGTAAGACA
CGACTTATCGCCACTGGCAGCAGCCACTGG
TAACAGGATTAGCAGAGCGAGGTATGTAGG
CGGTGCTACAGAGTTCTTGAAGTGGTGGCC
TAACTACGGCTACACTAGAAGAACAGTATT
TGGTATCTGCGCTCTGCTGAAGCCAGTTAC
CTTCGGAAAAAGAGTTGGTAGCTCTTGATC
CGGCAAACAAACCACCGCTGGTAGCGGTGG
TTTTTTTGTTTGCAAGCAGCAGATTACGCG
CAGAAAAAAAGGATCTCAAGAAGATCCTTT
GATCTTTTCTACGGGGTCTGACGCTCAGTG
GAACGAAAACTCACGTTAAGGGGCTCTTCA
GAAGAGCGAGTCACATTCGCGACTCTGCAA
TGCATTGCCATGAGATTATCAAAAAGGATC
TTCACCTAGATCCTTTTAAATTAAAAATGA
AGTTTTAAATCAATCTAAAGTATATATGAG
TAAACTTGGTCTGACAGTTACCAATGCTTA
ATCAGTGAGGCACCTATCTCAGCGATCTGT
CTATTTCGTTCATCCATAGTTGCCTGACTC
CCCGTCGTGTAGATAACTACGATACGGGAG
GGCTTACCATCTGGCCCCAGTGCTGCAATG
ATACCGCGAGACCCACGCTCACCGGCTCCA
GATTTATCAGCAATAAACCAGCCAGCCGGA
AGGGCCGAGCGCAGAAGTGGTCCTGCAACT
TTATCCGCCTCCATCCAGTCTATTAATTGT
TGCCGGGAAGCTAGAGTAAGTAGTTCGCCA
GTTAATAGTTTGCGCAACGTTGTTGCCATT
GCTACAGGCATCGTGGTGTCACGCTCGTCG
TTTGGTATGGCTTCATTCAGCTCCGGTTCC
CAACGATCAAGGCGAGTTACATGATCCCCC
ATGTTGTGCAAAAAAGCGGTTAGCTCCTTC
GGTCCTCCGATCGTTGTCAGAAGTAAGTTG
GCCGCAGTGTTATCACTCATGGTTATGGCA
GCACTGCATAATTCTCTTACTGTCATGCCA
TCCGTAAGATGCTTTTCTGTGACTGGTGAG
TACTCAACCAAGTCATTCTGAGAATAGTGT
ATGCGGCGACCGAGTTGCTCTTGCCCGGCG
TCAATACGGGATAATACCGCGCCACATAGC
AGAACTTTAAAAGTGCTCATCATTGGAAAA
CGTTCTTCGGGGCGAAAACTCTCAAGGATC
TTACCGCTGTTGAGATCCAGTTCGATGTAA
CCCACTCGTGCACCCAACTGATCTTCAGCA
TCTTTTACTTTCACCAGCGTTTCTGGGTGA
GCAAAAACAGGAAGGCAAAATGCCGCAAAA
AAGGGAATAAGGGCGACACGGAAATGTTGA
ATACTCATACTCTTCCTTTTTCAATATTAT
TGAAGCATTTATCAGGGTTATTGTCTCATG
AGCGGATACATATTTGAATGTATTTAGAAA
AATAAACAAATAGGGGTTCCGCGCACATTT
CCCCGAAAAGTA)

### 6.11 Example 11 Hairpinned Inverted Repeat DNA with Hetero-Telomeric HBOV Derived ITRs

A DNA fragment was de novo designed to contain double nicking sites (i) between the right HBOV-derived ITR, the pUC ori, (ii) between the pUC ori and the ampicillin resistance, as well as (iii) between the ampicillin resistance and the left HBOV-derived ITR and synthesized as well as cloned into a kanamycin resistance vector by a commercial vendor. Analogous to Example 10, the ITR and backbone fragment was then excised, and gel purified to be ligated with a firefly luciferase expression cassette fragment and circularized as described in the previous example. The plasmid is amplified, and 0.5 mg of plasmid are nicked with Nt.BspQI enzyme NEB (40 μl) at 500 ng/μl in nicking buffer and incubated for 1 h at 37 deg. The nicked plasmid is then denatured by addition of 33 mM of NaOH to release the overhangs, followed by annealing of the overhangs with 41.6 mM Tris pH 7.2. Then the annealed DNA fragments are digested with RecBCD. Samples of each step care visualized an agarose gel to confirm the production of the vector.

(SEQ ID NO: 338)
GTTCGAGATCGCTCTTCAGAAGAGCGCGGA
GTCACATTCGCGACTCGCAATGCATTGCAG
GGCGAGGGCGAGGGCCGCCCCTACGAGGGC
CACAACACCGTGAAGCTGAAGGTGACCAAG
GGCGGCCCCCTGCCCTTCGCCTGGGACATC
CTGTCCCCCCAGTTCCAGTACGGCTCCAAG
GTGTACGTGAAGCACCCCGCCGACATCCCA
GCTCGGTAGCCACCTGACGTCGCTCTTCGG
TGGTTGTACAGACGCCATCTTGGAATCCAA
TATGTCTGCCGGCTCAGTCATGCCTGCGCT
GCGCGCAGCGCGCTGCGCGCGCGCATGATC
TAATCGCCGGCAGACATATTGGATTCCAAG
ATGGCGTCTGTACAACCACCGAAGAGCGGA
GTGGCCAAAAAGTACCCGGCCAGAAAGGAA
CCGTAAAACCAACTAGACAGCCGGTGACTT
TGTTCCCCAGCTCGGTTCATTCTCAAGCCT
CAGACAGGAGAACCGGGACTAGTATATGAG
CTCCGGTGCCCGTCAGTGGGCAGAGCGCAC
ATCGCCCACAGTCCCCGAGAAGTTGGGGGG
AGGGGTCGGCAATTGAACCGGTGCCTAGAG
AAGGTGGCGCGGGGTAAACTGGGAAAGTGA
TGTCGTGTACTGGCTCCGCCTTTTTCCCGA
GGGTGGGGGAGAACCGTATATAAGTGCAGT
AGTCGCCGTGAACGTTCTTTTTCGCAACGG
GTTTGCCGCCAGAACACAGGTAAGTGCCGT
GTGTGGTTCCCGCGGGCCTGGCCTCTTTAC
GGGTTATGGCCCTTGCGTGCCTTGAATTAC
TTCCACGCCCCTGGCTGCAGTACGTGATTC
TTGATCCCGAGCTTCGGGTTGGAAGTGGGT
GGGAGAGTTCGAGGCCTTGCGCTTAAGGAG
CCCCTTCGCCTCGTGCTTGAGTTGAGGCCT
GGCCTGGGCGCTGGGGCCGCCGCGTGCGAA
TCTGGTGGCACCTTCGCGCCTGTCTCGCTG
CTTTCGATAAGTCTCTAGCCATTTAAAATT
TTTGATGACCTGCTGCGACGCTTTTTTTCT
GGCAAGATAGTCTTGTAAATGCGGGCCAAG
ATCGATCTGCACACTGGTATTTCGGTTTTT
GGGGCCGCGGGCGGCGACGGGGCCCGTGCG
TCCCAGCGCACATGTTCGGCGAGGCGGGGC
CTGCGAGCGCGGCCACCGAGAATCGGACGG
GGGTAGTCTCAAGCTGGCCGGCCTGCTCTG
GTGCCTGGCCTCGCGCCGCCGTGTATCGCC
CCGCCCTGGGCGGCAAGGCTGGCCCGGTCG
GCACCAGTTGCGTGAGCGGAAAGATGGCCG
CTTCCCGGCCCTGCTGCAGGGAGCTCAAAA
TGGAGGACGCGGCGCTCGGGAGAGCGGGCG
GGTGAGACACCCACACAAAGGAAAAGGGCC
TTTCCGTCCTCAGCCGTCGCTTCATGTGAC
ACCACGGAGTACCGGGCGCCGTCCAGGCAC
CTCGATTAGTTCTCGATCGAGCTTTTGGAG
TACGTCGTCTTTAGGTTGGGGGGAGGGGTT
TTATGCGATGGAGTTTCCCCACACTGAGTG
GGTGGAGACTGAAGTTAGGCCAGCTTGGCA
CTTGATGTAATTCTCCTTGGAATTTGCCCT
TTTTGAGTTTGGATCTTGGTTCATTCTCAA
GCCTCAGACAGTGGTTCAAAGTTTTTTTCT
TCCATTTCAGGTGTCGTGAGCGGCCGCGGC
AGGCTGCCACCATGGAGGATGCCAAGAATA
TTAAGAAAGGCCCTGCCCCATTCTACCCTC
TGGAAGATGGCACTGCTGGTGAGCAACTGC
ACAAGGCCATGAAGAGGTATGCCCTGGTCC
CTGGCACAATTGCCTTCACTGATGCTCACA
TTGAGGTGGACATCACCTATGCTGAATACT
TTGAGATGTCTGTGAGGCTGGCAGAAGCCA
TGAAAAGATATGGACTGAACACCAACCACA
GGATTGTGGTGTGCTCTGAGAACTCTCTCC
AGTTCTTCATGCCTGTGTTGGGAGCCCTGT
TCATTGGAGTGGCTGTGGCCCCTGCCAATG
ACATCTACAATGAGAGAGAGCTCCTGAACA
GCATGGGCATCAGCCAGCCAACTGTGGTCT
TTGTGAGCAAGAAGGGCCTGCAAAAGATCC
TGAATGTGCAGAAGAAGCTGCCCATCATCC
AGAAGATCATCATCATGGACAGCAAGACTG
ACTACCAGGGCTTCCAGAGCATGTATACCT
TTGTGACCAGCCACCTCCCCCCTGGCTTCA
ATGAGTATGACTTTGTGCCTGAGAGCTTTG
ACAGGGACAAGACAATTGCTCTGATTATGA
ACAGCTCTGGCTCCACTGGACTGCCCAAAG
GTGTGGCTCTGCCCCACAGAACTGCTTGTG
TGAGATTCAGCCATGCCAGAGACCCCATCT
TTGGCAACCAGATCATCCCTGACACTGCCA
TCCTGTCTGTGGTTCCATTCCATCATGGCT
TTGGCATGTTCACAACACTGGGGTACCTGA
TCTGTGGCTTCAGAGTGGTGCTGATGTATA
GGTTTGAGGAGGAGCTGTTTCTGAGGAGCT
TGCAAGACTACAAGATCCAGTCTGCCCTGC
TGGTGCCCACTCTGTTCAGCTTCTTTGCCA
AGAGCACCCTCATTGACAAGTATGACCTGA
GCAACCTGCATGAGATTGCCTCTGGAGGAG
CACCCCTGAGCAAGGAGGTGGGTGAGGCTG
TGGCAAAGAGGTTCCATCTCCCAGGAATCA
GACAGGGCTATGGCCTGACTGAGACCACCT
CTGCCATCCTCATCACCCCTGAAGGAGATG
ACAAGCCTGGTGCTGTGGGCAAGGTGGTTC
CCTTTTTTGAGGCCAAGGTGGTGGACCTGG
ACACTGGCAAGACCCTGGGAGTGAACCAGA
GGGGTGAGCTGTGTGTGAGGGGTCCCATGA
TCATGTCTGGCTATGTGAACAACCCTGAGG
CCACCAATGCCCTGATTGACAAGGATGGCT
GGCTGCACTCTGGTGATATTGCCTACTGGG
ATGAGGATGAGCACTTTTTCATTGTGGACA
GGCTGAAGAGTCTCATCAAGTACAAAGGCT
ACCAAGTGGCACCTGCTGAGCTTGAGAGCA
TCCTGCTCCAGCACCCCAACATCTTTGATG
CTGGTGTGGCTGGCCTGCCTGATGATGATG
CTGGAGAGCTGCCTGCTGCTGTTGTGGTTC
TGGAGCATGGAAAGACCATGACTGAGAAGG
AGATTGTGGACTATGTGGCCAGTCAGGTGA
CCACTGCCAAGAAGCTGAGGGGAGGTGTGG
TGTTTGTGGATGAGGTGCCAAAGGGTCTGA
CTGGCAAGCTGGATGCCAGAAAGATCAGAG
AGATCCTGATCAAGGCCAAGAAGGGAGGAA
AGATTGCAGTTTAAGGATCCTGACACAAAG
TGACGCGTAATCAACCTCTGGATTACAAAA
TTTGTGAAAGATTGACTGGTATTCTTAACT
ATGTTGCTCCTTTTACGCTATGTGGATACG
CTGCTTTAATGCCTTTTATCATGCTATTGC
TTGCCCGTATGGCTTTCATTTTCTCCTCCT
TGTATAAATCCTGGTTGCTGTCTCTTTATG
AGGAGTTGTGGCCCGTTGTCAGGCAACGTG
GCGTGGTGTGCACTGTGTTTGCTGACGCAA
CCCCCACTGGTTGGGGCTTTGCCACCACCT
GTCAGCTCCTTTCCGGGACTTTCGCTTTCC
CCCTCCCTATTGCCACGGCGGAACTCATCG
CCGCCTGCCTTGCCCGCTGCTGGACAGGGG
CTCGGCTGTTGGGCACTGACAATTCCGTGG
TGTTGTCGGGGAAATCATCGTCCTTTCCTT
GGCTGCTCGCCTGTGTTGCCACCTGGATTC
TGCGCGGGACGTCCTTCTGCTACGTCCCTT
CGGCCCTCAATCCAGCGGACCTTCCTTCCC
GCGGCCTGCTGCCGGCTCTGCGGCCTCTTC
CGCGTCTTCGCCTTCGCCCTCAGACGAGTC
GGATCTCCCTTTGGGCCGCCTCCCCGCACG
CGTCCTAGAGCTCGCACTGTGCCTTCTAGT
TGCCAGCCATCTGTTGTTTGCCCCTCCCCC
GTGCCTTCCTTGACCCTGGAAGGTGCCACT
CCCACTGTCCTTTCCTAATAAAATGAGGAA
ATTGCATCGCATTGTCTGAGTAGGTGTCAT
TCTATTCTGGGGGGTGGGGTGGGGCAGGAC
AGCAAGGGGGAGGATTGGGAAGAGAATAGC
AGGCATGCTGGGGAGGGCGCTAGCGCAGGG
GGACCCCAAGTGACGATTGAACCCCGATTC
AGGTACCCATTGGAAATTTTAATGGAGTAA
CATAACTGCTGGAGTAGATGAAGAACCTCT
TGGCCACTCCGTTGCTTATGCAATCGCGAA
ACTCTATATCTTGCTCTTCTTAATGTGTTG
TTGTTGTACATGCGCCATCTTAGTTTTATA
TCAGCTGGCGCCTTAGTTATATAACATGCA
TGTTATATAACTAAGGCGCCAGCTGATATA
AAACTAAGATGGCGCATGTACAACAACAAC
ACATTAAGAAGAGCGCCGGGGGGACGACGC
AATCTGGAGTGGAAAGAACCCCCGTCTATG
CGGCTTAAAGCACGGCCAGGGAATAGTGGA
TCAAGTGTACTGACATGTGCCGGCAATGCA
TTGCGTCCCGAGTCACATTCGCGACTCTCG
ACGCTCTTCAGAAGAGCAGCTTAGCTTCAA
TAGCTCAATGATATCGGAAAGAACATGTGA
GCAAAAGGCCAGCAAAAGGCCAGGAACCGT
AAAAAGGCCGCGTTGCTGGCGTTTTTCCAT
AGGCTCCGCCCCCCTGACGAGCATCACAAA
AATCGACGCTCAAGTCAGAGGTGGCGAAAC
CCGACAGGACTATAAAGATACCAGGCGTTT
CCCCCTGGAAGCTCCCTCGTGCGCTCTCCT
GTTCCGACCCTGCCGCTTACCGGATACCTG
TCCGCCTTTCTCCCTTCGGGAAGCGTGGCG
CTTTCTCATAGCTCACGCTGTAGGTATCTC
AGTTCGGTGTAGGTCGTTCGCTCCAAGCTG
GGCTGTGTGCACGAACCCCCCGTTCAGCCC
GACCGCTGCGCCTTATCCGGTAACTATCGT
CTTGAGTCCAACCCGGTAAGACACGACTTA
TCGCCACTGGCAGCAGCCACTGGTAACAGG
ATTAGCAGAGCGAGGTATGTAGGCGGTGCT
ACAGAGTTCTTGAAGTGGTGGCCTAACTAC
GGCTACACTAGAAGAACAGTATTTGGTATC
TGCGCTCTGCTGAAGCCAGTTACCTTCGGA
AAAAGAGTTGGTAGCTCTTGATCCGGCAAA
CAAACCACCGCTGGTAGCGGTGGTTTTTTT
GTTTGCAAGCAGCAGATTACGCGCAGAAAA
AAAGGATCTCAAGAAGATCCTTTGATCTTT
TCTACGGGGTCTGACGCTCAGTGGAACGAA
AACTCACGTTAAGGGGCTCTTCAGAAGAGC
GAGTCACATTCGCGACTCTGCAATGCATTG
CCATGAGATTATCAAAAAGGATCTTCACCT
AGATCCTTTTAAATTAAAAATGAAGTTTTA
AATCAATCTAAAGTATATATGAGTAAACTT
GGTCTGACAGTTACCAATGCTTAATCAGTG
AGGCACCTATCTCAGCGATCTGTCTATTTC
GTTCATCCATAGTTGCCTGACTCCCCGTCG
TGTAGATAACTACGATACGGGAGGGCTTAC
CATCTGGCCCCAGTGCTGCAATGATACCGC
GAGACCCACGCTCACCGGCTCCAGATTTAT
CAGCAATAAACCAGCCAGCCGGAAGGGCCG
AGCGCAGAAGTGGTCCTGCAACTTTATCCG
CCTCCATCCAGTCTATTAATTGTTGCCGGG
AAGCTAGAGTAAGTAGTTCGCCAGTTAATA
GTTTGCGCAACGTTGTTGCCATTGCTACAG
GCATCGTGGTGTCACGCTCGTCGTTTGGTA
TGGCTTCATTCAGCTCCGGTTCCCAACGAT
CAAGGCGAGTTACATGATCCCCCATGTTGT
GCAAAAAAGCGGTTAGCTCCTTCGGTCCTC
CGATCGTTGTCAGAAGTAAGTTGGCCGCAG
TGTTATCACTCATGGTTATGGCAGCACTGC
ATAATTCTCTTACTGTCATGCCATCCGTAA
GATGCTTTTCTGTGACTGGTGAGTACTCAA
CCAAGTCATTCTGAGAATAGTGTATGCGGC
GACCGAGTTGCTCTTGCCCGGCGTCAATAC
GGGATAATACCGCGCCACATAGCAGAACTT
TAAAAGTGCTCATCATTGGAAAACGTTCTT
CGGGGCGAAAACTCTCAAGGATCTTACCGC
TGTTGAGATCCAGTTCGATGTAACCCACTC
GTGCACCCAACTGATCTTCAGCATCTTTTA
CTTTCACCAGCGTTTCTGGGTGAGCAAAAA
CAGGAAGGCAAAATGCCGCAAAAAAGGGAA
TAAGGGCGACACGGAAATGTTGAATACTCA
TACTCTTCCTTTTTCAATATTATTGAAGCA
TTTATCAGGGTTATTGTCTCATGAGCGGAT
ACATATTTGAATGTATTTAGAAAAATAAAC
AAATAGGGGTTCCGCGCACATTTCCCCGAA
AAGTA

### 6.12 Example 12: Production of Constructs Encoding the FVIII

The nucleic acid sequences encoding the FVIII expression cassette were designed in silico. The construct encodes for a modified left ITR devoid of RABS, a modified EF-1a promoter, a truncated FVIII ORF (SEQ ID 180), bGH poly (a), a modified right ITR devoid of RABS and a double restriction sites for nicking endonuclease 250 base pairs downstream of the right ITR

(SEQ ID NO: 343)
(GAATTCGAGCTCGGTAGCCGAAGAGCGCG
CTCGCTATCTCACTGAGGCCGGGCGACCAA
AGGTCGCCCGACGCCCGGGCTTTGCCCGGG
CGGCCTCAGTGAGATAGCGAGCGCGCTCTT
CGGGAGTGGCCAAAACCAACTAGACAACTT
TGTATATCTAGAGCTCCGGTGCCCGTCAGT
GGGCAGAGCGCACATCGCCCACAGTCCCCG
AGAAGTTGGGGGGAGGGGTCGGCAATTGAA
CCGGTGCCTAGAGAAGGTGGCGCGGGGTAA
ACTGGGAAAGTGATGTCGTGTACTGGCTCC
GCCTTTTTCCCGAGGGTGGGGGAGAACCGT
ATATAAGTGCAGTAGTCGCCGTGAACGTTC
TTTTTCGCAACGGGTTTGCCGCCAGAACAC
AGGTAAGTGCCGTGTGTGGTTCCCGCGGGC
CTGGCCTCTTTACGGGTTATGGCCCTTGCG
TGCCTTGAATTACTTCCACGCCCCTGGCTG
CAGTACGTGATTCTTGATCCCGAGCTTCGG
GTTGGAAGTGGGTGGGAGAGTTCGAGGCCT
TGCGCTTAAGGAGCCCCTTCGCCTCGTGCT
TGAGTTGAGGCCTGGCCTGGGCGCTGGGGC
CGCCGCGTGCGAATCTGGTGGCACCTTCGC
GCCTGTCTCGCTGCTTTCGATAAGTCTCTA
GCCATTTAAAATTTTTGATGACCTGCTGCG
ACGCTTTTTTTCTGGCAAGATAGTCTTGTA
AATGCGGGCCAAGATCGATCTGCACACTGG
TATTTCGGTTTTTGGGGCCGCGGGCGGCGA
CGGGGCCCGTGCGTCCCAGCGCACATGTTC
GGCGAGGCGGGGCCTGCGAGCGCGGCCACC
GAGAATCGGACGGGGGTAGTCTCAAGCTGG
CCGGCCTGCTCTGGTGCCTGGCCTCGCGCC
GCCGTGTATCGCCCCGCCCTGGGCGGCAAG
GCTGGCCCGGTCGGCACCAGTTGCGTGAGC
GGAAAGATGGCCGCTTCCCGGCCCTGCTGC
AGGGAGCTCAAAATGGAGGACGCGGCGCTC
GGGAGAGCGGGCGGGTGAGACACCCACACA
AAGGAAAAGGGCCTTTCCGTCCTCAGCCGT
CGCTTCATGTGACACCACGGAGTACCGGGC
GCCGTCCAGGCACCTCGATTAGTTCTCGAT
CGAGCTTTTGGAGTACGTCGTCTTTAGGTT
GGGGGGAGGGGTTTTATGCGATGGAGTTTC
CCCACACTGAGTGGGTGGAGACTGAAGTTA
GGCCAGCTTGGCACTTGATGTAATTCTCCT
TGGAATTTGCCCTTTTTGAGTTTGGATCTT
GGTTCATTCTCAAGCCTCAGACAGTGGTTC
AAAGTTTTTTTCTTCCATTTCAGGTGTCGT
GAGCGGCCGCGGCAGGCTGCCACCATGCAG
ATTGAACTGTCTACCTGCTTCTTTCTGTGC
CTCCTGAGGTTCTGTTTTAGTGCCACAAGG
AGATATTACCTAGGAGCAGTTGAGCTGTCC
TGGGATTACATGCAGAGTGATCTGGGGGAG
CTGCCAGTTGATGCTAGGTTCCCTCCAAGG
GTGCCTAAGTCCTTCCCCTTTAATACCTCA
GTGGTGTATAAGAAGACCCTTTTTGTAGAG
TTCACTGATCACCTGTTCAATATAGCTAAA
CCCAGGCCACCTTGGATGGGGTTGTTGGGC
CCAACAATTCAGGCAGAGGTTTATGACACA
GTAGTGATAACCCTCAAGAATATGGCTAGT
CATCCTGTGAGCCTGCATGCAGTAGGTGTG
TCTTACTGGAAGGCTTCAGAGGGTGCTGAG
TATGATGATCAGACTTCTCAGAGAGAGAAG
GAAGATGACAAAGTCTTTCCAGGGGGATCA
CATACATATGTTTGGCAGGTTCTTAAAGAG
AATGGTCCTATGGCCAGTGACCCCCTGTGT
CTGACTTACTCCTACCTGAGCCATGTGGAC
CTGGTGAAGGATCTGAACTCTGGGCTGATA
GGTGCACTGCTGGTGTGTAGGGAAGGTAGC
CTTGCCAAAGAGAAGACCCAAACACTCCAC
AAGTTCATCTTGCTCTTTGCAGTTTTTGAT
GAAGGAAAAAGCTGGCATAGTGAAACCAAA
AACTCTTTGATGCAGGATAGGGATGCAGCT
AGTGCTAGGGCCTGGCCAAAGATGCACACA
GTCAATGGTTATGTGAACAGAAGTCTTCCA
GGTCTGATAGGATGCCACAGGAAAAGTGTG
TACTGGCATGTGATAGGCATGGGAACCACC
CCAGAAGTGCACTCAATATTCCTTGAGGGT
CATACCTTCCTTGTGAGAAATCATAGGCAG
GCATCCTTGGAGATCAGTCCCATCACCTTC
TTAACTGCCCAAACCCTTCTGATGGACCTT
GGTCAATTTCTGCTGTCCTGCCATATTTCT
TCCCATCAGCATGATGGGATGGAGGCCTAT
GTAAAGGTGGATTCTTGCCCAGAGGAACCC
CAGCTGAGGATGAAGAACAATGAGGAAGCA
GAGGATTATGATGATGATCTTACTGATAGT
GAGATGGATGTGGTTAGGTTTGATGATGAC
AACAGCCCCAGCTTTATCCAGATTAGGTCT
GTTGCTAAGAAGCATCCAAAGACATGGGTT
CATTACATAGCTGCAGAAGAAGAGGACTGG
GATTATGCCCCCCTAGTCCTTGCCCCAGAT
GATAGATCCTATAAATCTCAGTATCTGAAC
AATGGACCTCAGAGGATAGGCAGGAAGTAT
AAGAAAGTGAGATTCATGGCATATACAGAT
GAGACCTTCAAGACCAGAGAAGCAATCCAA
CATGAAAGTGGGATTCTGGGCCCACTGCTG
TATGGTGAGGTGGGTGACACCCTGCTGATA
ATCTTCAAGAATCAGGCCAGCAGGCCTTAT
AACATCTATCCACATGGGATAACAGATGTA
AGGCCTCTGTATAGTAGAAGGCTGCCCAAA
GGGGTGAAACACCTGAAAGACTTTCCTATT
CTTCCAGGTGAAATCTTCAAATACAAGTGG
ACAGTCACAGTGGAGGATGGGCCTACTAAA
AGTGACCCCAGGTGCCTGACTAGGTACTAT
AGTTCCTTTGTTAACATGGAAAGGGACCTA
GCTAGTGGGCTTATAGGCCCCCTTCTGATC
TGTTATAAAGAATCTGTTGATCAGAGGGGC
AACCAGATCATGTCAGACAAAAGGAATGTG
ATACTTTTTAGTGTGTTTGATGAGAATAGG
AGCTGGTACCTGACTGAGAACATCCAGAGG
TTTTTGCCCAACCCAGCTGGTGTGCAGCTT
GAGGACCCAGAGTTTCAAGCCTCAAACATC
ATGCACAGTATCAATGGCTATGTTTTTGAT
TCTCTTCAGCTGAGTGTTTGCCTGCATGAA
GTAGCTTACTGGTACATCCTCTCAATTGGG
GCTCAGACAGATTTCCTTTCAGTGTTCTTC
AGTGGCTACACTTTCAAGCACAAAATGGTC
TATGAGGACACTCTGACCTTATTCCCTTTT
AGTGGAGAGACAGTCTTTATGAGTATGGAG
AATCCTGGGCTGTGGATTCTTGGGTGCCAC
AACTCTGATTTCAGAAATAGGGGAATGACT
GCCCTGCTAAAGGTGAGTAGTTGTGACAAA
AACACAGGGGATTACTATGAGGACAGCTAT
GAGGACATCAGTGCCTATCTGCTCTCAAAG
AACAATGCAATAGAGCCTAGGTCCTTCTCA
CAGAATAGCAGGCATCCCTCAACCAGGCAA
AAACAGTTTAATGCCACCACCATACCAGAG
AATGATATTGAAAAGACTGACCCTTGGTTT
GCTCACAGGACCCCAATGCCCAAAATCCAA
AATGTGTCTTCAAGTGACCTGTTAATGCTG
TTGAGGCAGTCACCCACCCCACATGGGCTG
TCCCTGTCAGATCTTCAAGAGGCCAAATAT
GAGACCTTCAGTGATGATCCTTCACCAGGA
GCCATAGATTCTAACAACTCCCTGTCAGAA
ATGACACACTTCAGACCTCAGTTACATCAT
TCTGGGGATATGGTGTTCACACCTGAATCT
GGTCTCCAGCTGAGGTTAAATGAGAAACTT
GGGACTACAGCAGCTACTGAGCTGAAAAAA
CTTGATTTTAAGGTGAGCTCAACAAGCAAT
AACCTGATTTCCACCATTCCTAGTGACAAC
CTAGCTGCTGGCACTGATAACACCTCTTCT
CTGGGCCCTCCTTCAATGCCAGTACATTAT
GATTCCCAACTTGATACCACCCTCTTTGGG
AAGAAGTCTAGCCCACTTACAGAATCAGGT
GGCCCACTTAGTCTCTCAGAGGAGAATAAT
GATTCCAAACTTTTGGAGAGTGGGCTGATG
AACTCTCAAGAGAGCAGTTGGGGGAAGAAT
GTATCCCAGAGAGAGATAACCAGAACAACC
CTTCAGTCTGACCAGGAGGAGATAGATTAT
GATGACACTATCTCAGTTGAGATGAAAAAG
GAGGACTTTGATATCTATGATGAAGATGAA
AATCAGTCTCCCAGGTCCTTCCAGAAAAAA
ACAAGGCATTACTTCATAGCAGCAGTAGAG
AGACTGTGGGATTATGGGATGTCATCCAGT
CCTCATGTCCTGAGGAACAGAGCTCAGAGT
GGCTCAGTGCCTCAATTCAAGAAAGTGGTG
TTCCAGGAGTTCACAGATGGCTCCTTCACA
CAGCCTCTCTACAGAGGAGAGCTCAATGAA
CACTTGGGGCTGCTGGGGCCCTATATCAGG
GCAGAGGTGGAGGATAATATTATGGTGACA
TTTAGGAACCAAGCCTCAAGGCCATACTCA
TTTTATTCTTCCCTGATCTCATATGAAGAG
GATCAGAGGCAGGGGGCAGAACCTAGGAAG
AATTTTGTAAAGCCTAATGAGACTAAGACT
TACTTTTGGAAGGTGCAACACCACATGGCT
CCCACCAAAGATGAATTTGATTGCAAGGCT
TGGGCCTACTTCTCTGATGTGGATCTGGAG
AAAGATGTGCATTCTGGCTTGATAGGTCCC
CTACTGGTTTGTCATACAAATACCCTGAAC
CCTGCACATGGCAGGCAGGTGACTGTGCAG
GAGTTTGCTCTCTTCTTCACTATATTTGAT
GAGACCAAGTCCTGGTACTTCACAGAGAAT
ATGGAAAGAAACTGCAGGGCCCCATGTAAT
ATCCAGATGGAAGACCCCACCTTTAAGGAA
AATTATAGGTTTCATGCTATTAATGGATAT
ATAATGGACACCCTCCCAGGTCTGGTCATG
GCACAGGACCAGAGGATAAGGTGGTATCTG
CTTTCTATGGGCTCTAATGAAAATATTCAT
AGCATCCACTTTAGTGGGCATGTGTTCACT
GTCAGGAAAAAAGAGGAGTATAAAATGGCC
CTGTATAACCTGTACCCAGGAGTGTTTGAG
ACAGTAGAGATGCTTCCTAGTAAGGCTGGC
ATTTGGAGAGTAGAGTGCCTGATTGGAGAA
CATCTCCATGCTGGAATGAGCACCCTGTTC
CTGGTCTATAGTAACAAGTGTCAGACCCCA
CTGGGCATGGCCTCAGGTCATATTAGGGAC
TTCCAAATCACTGCCAGTGGCCAATATGGG
CAGTGGGCCCCCAAACTAGCCAGGTTGCAC
TATTCTGGGAGTATTAATGCCTGGTCTACA
AAGGAACCATTTTCCTGGATCAAAGTGGAT
CTGCTGGCCCCAATGATCATACATGGAATT
AAGACCCAGGGAGCCAGGCAGAAGTTTTCC
TCCCTGTACATTAGTCAGTTCATCATTATG
TACAGTTTGGATGGGAAAAAGTGGCAGACA
TATAGGGGTAACAGTACAGGAACCCTTATG
GTGTTCTTTGGTAATGTTGACAGCTCAGGA
ATCAAGCACAATATTTTCAACCCCCCAATC
ATAGCCAGGTACATCAGGCTCCACCCTACT
CATTATAGCATCAGGAGCACCCTGAGAATG
GAGCTTATGGGATGTGACCTAAACTCCTGT
AGTATGCCTCTGGGAATGGAATCAAAGGCC
ATCTCTGATGCCCAAATTACAGCCTCATCC
TACTTTACAAACATGTTTGCTACTTGGTCA
CCATCCAAAGCTAGACTGCACCTGCAGGGG
AGGTCAAATGCCTGGAGGCCACAAGTCAAC
AACCCCAAAGAATGGCTCCAAGTTGACTTT
CAGAAGACTATGAAAGTGACTGGGGTGACT
ACTCAAGGGGTAAAGTCTCTTCTTACCAGT
ATGTATGTCAAAGAGTTTTTGATAAGCTCC
TCCCAGGATGGCCACCAGTGGACACTGTTT
TTTCAGAATGGCAAGGTGAAAGTGTTCCAA
GGAAACCAGGATTCTTTTACTCCAGTGGTG
AACAGCCTGGACCCCCCCCTTCTGACCAGA
TACTTGAGAATCCACCCCCAGTCCTGGGTT
CATCAGATTGCTCTGAGGATGGAAGTGCTG
GGATGTGAGGCCCAGGACCTCTACTAAGGA
TCCTGACACAAAGTGACGCGTCCTAGAGCT
CGCACTGTGCCTTCTAGTTGCCAGCCATCT
GTTGTTTGCCCCTCCCCCGTGCCTTCCTTG
ACCCTGGAAGGTGCCACTCCCACTGTCCTT
TCCTAATAAAATGAGGAAATTGCATCGCAT
TGTCTGAGTAGGTGTCATTCTATTCTGGGG
GGTGGGGTGGGGCAGGACAGCAAGGGGGAG
GATTGGGAAGAGAATAGCAGGCATGCTGGG
GAGGGCGCTAGCGCAGGAACCCCTTTTAAT
GGAGGGCCACTCCCGAAGAGCGCGCTCGCT
ATCTCACTGAGGCCGGGCGACCAAAGGTCG
CCCGACGCCCGGGCTTTGCCCGGGCGGCCT
CAGTGAGATAGCGAGCGCGCTCTTCGGGCG
CAAGCTTGGCGTAATCATGGTCATAGCTGT
TTCCTGTGTGAAATTGTTATCCGCTCACAA
TTCCACACAACATACGAGCCGGAAGCATAA
AGTGTAAAGCCTGGGGTGCCTAATGAGTGA
GCTAACTCACATTAATTGCGTTGCGCTCAC
TGCCCGCTTTCCAGTCGGGAAACCTGTCGT
GCCAGCTGCATTAATGAATCGGCCAACGCG
CGGGGAGAGGCGGTTTGCGTATTGGGCGCT
CTTCCGGGGAAGAGCC.

As second construct was designed in which the ORF of SEQ ID NO 343 was replaced with a full length FVIII CpG minimized sequence (SEQ ID NO: 175). The constructs were synthetized and cloned into backbone comprising a pUC Ori and an antibiotic resistance by a commercial DNA synthesis vendor.

The plasmids were transformed and then amplified overnight in the NEBstable or MDS-42 strain, followed by plasmid isolation using commercial plasmid isolation kit (Nucleobond Xtra Maxi Plus EF (Macherey Nagel)) and dissolved in TE buffer.

To produce the hairpin ended DNA, the plasmids were nicked with the nicking endonuclease Nt.BspQI (6.2 U/μg DNA) was added to the isolated construct 1 in 1× Neb3.1 Buffer and incubated at 55° C. for one hour. The reaction mix containing the nicked plasmid was then heated to 95° C. on a thermo shaker for 3 min, in order to dissociate the ITR flanked transgene from the plasmid back bone and the mix was then left to cool to room temperature for 30 min to allow for ITR folding at the single stranded overhangs ends. The reaction mix was then supplemented with and RecBCD Exonuclease V (0.625 U per μg of nicked plasmid) as well as adenosine triphosphate (final concentration of 1 mM). The reaction mix was then placed on a shaker at 37° C. for 120 min to allow for the restriction enzyme to cleave the backbone fragment and the exonuclease to digest backbone fragments. The exonuclease generally does not digest linear fragments protected by closed ends. Finally, the reaction mix was purified using Takara NucleoSpin Gel and PCR clean-up kit and remaining ITR flanked vector was eluted according to the manufacturer's instructions.

Nicked, de/renatured and digestion resistant DNA products were visualized by native agarose gel electrophoresis. For the DNA construct encoding the truncated FVIII, the agarose gel (FIG. 25A) shows the nicked plasmid in lane 2, the de/renatured DNA products in lane 3, a single band of digestion resistant vector in lane 4 and the purified product in lane 5. For the DNA construct encoding the full length FVIII, the agarose gel (FIG. 25B) shows the nicked plasmid in lane 2, the de/renatured DNA products in lane 3, a single band of digestion resistant vector in lane 4 and the purified product in lane 5.

### 6.13 Example 13: FVIII Activity Assay

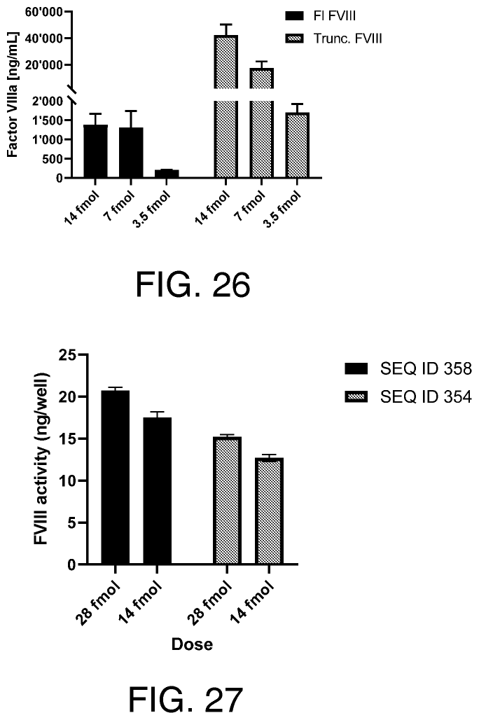

To determine the level of FVIII activity, 10′000 HEK293T cells were transfected with 14 fmol, 7 fmol and 3.5 fmol of hairpinned DNA molecules encoding truncated and full length FVIII of Example 12. Constructs were transfected using JetPrime according to according to manufacturer's manual. After 72 h the transfection the Coagulation factor VIII in the supernatants was analyzed by Factor VIIIa activity assay kit (fluorometric, Abcam ab204696) according to manufacturer's manual and is shown in FIG. 26.

### 6.14 Example 14: Activity CpG Free ITR Construct Encoding Truncated FVIII Variants

In order to demonstrate the additional utility of preparing hairpin ended DNA vectors provided herein, without the need for RAPs and consequently independent of the RABS and/or TRS for genome DNA replication was well as any other viral sequence; genetic expression systems were generated flanked with T-shaped ITRs comprising CpG-free ITRs. The CpG free ITR constructs comprised a modified CpG free left ITR (SEQ ID 344), a modified right CpG free ITR (SEQ ID 345), a liver specific promoter and a CpG free polyadenylation signal. Two constructs were generated by cloning in a transgene region encoding truncated versions of FVIII, one with a partial B-domain deletion (deletion of amino acids 984-1665 of the FVIII wild type sequence) and one with the same partial B-domain deletion as well as a N-terminal linker a3 deletion (deletion of amino acids 984-1677 of the wildtype sequence) (SEQ ID NOs: 354 and 358, respectively). The open reading frame sequence was further depleted of CpG and nicking sites. The hairpin ended DNA molecules were prepared and purified as described above in Example 12.

To determine the level of FVIII expression, 20′000 HUH-7 cells were transfected with 28 fmol and 14 fmol hairpinned DNA molecules encoding two versions of truncated FVIII (SEQ ID NOs: 358 and 354). Constructs were transfected using JetPrime according to the manufacturer's manual. After 48 h the transfection of the Coagulation factor VIII in the supernatants was analyzed by Factor VIIIa activity assay kit (fluorometric, Abcam ab204696) according to the manufacturer's manual and the results are shown in FIG. 27. The construct with the partially deleted linker a3 exhibited increased Factor VIII activity compared to the B-domain deleted construct.

| | |
|---|---|
| L- | AGAAGAGCTCTCTGTACACTCACTCAC |
| ITR | TCACTGATCCCTGGATACCAAAGGTAT |
| CpG | CCAGACACCCAGTCTTTGACTGGGTGG |
| Free | GATCAGTGAGTGAGTGAGTGTACAGAG |
| | AGCTCTTCT |
| | (SEQ ID NO: 344) |
| R- | GCTCTTCTTCTCTGTACACTCACTCAC |
| ITR | TCACTGATCCCACCCAGTCAAAGACTG |
| CpG | GGTGTCTGGATACCTTTGGTATCCAGG |
| Free | GATCAGTGAGTGAGTGAGTGTACAGAG |
| | AAGAAGAGCT |
| | (SEQ ID NO: 345) |

### 6.15 Example 15: Treatment of Hemophilia a with Hairpin-Ended FVIII DNA Constructs

A hairpin-ended DNA encoding FVIII, described herein, is deemed useful for treatment of Hemophilia A when expressed as a transgene. A subject presenting with Hemophilia A is administered a hairpin ended DNA-based vector that encodes FVIII intravenously at a dose sufficient to deliver and maintain a therapeutically effective concentration of FVIII protein. Following treatment, the subject is evaluated for improvement in symptoms of Hemophilia A. The ability of the hairpin ended DNA-based vector to increase plasma FVIII levels is determined after 72 h.

### 6.16 Example 16: Treatment of Hemophilia A in Animals Models with FVIII

A human FVIII-based vector is deemed useful for treatment of Hemophilia A when expressed as a transgene. An animal model for Hemophilia A, for example animal models described in Yen, C T., Fan, M N., Yang, Y L. et al. Current animal models of hemophilia: the state of the art. Thrombosis J 14, 22 (2016), is administered a hairpin-ended DNA molecule described herein that encodes FVIII intravenously at a dose sufficient to deliver and maintain a therapeutically effective concentration of FVIII protein. Following treatment, the animal is evaluated for improvement in symptoms consistent with the disease in the particular animal model. The ability of the hairpin ended DNA-based vector to increase FVIII levels in plasma after 72 hours is determined.

Long term expression can be determined as follows: 8-12-weeks old HemeA mice are administered either LNP-construct encoding therapeutic FVIII, or LNP-pDNA (plasmid control) at 0.3, 0.5, 1, and 2 mg/kg via IV injection, N=4/group. Blood samples are collected at selected time points starting at 48 hours post-injection for up to 3 months and FVIII activity in blood is analyzed by the chromogenic FVIII activity assay. FVIII expression profile in mice treated with LNP-construct can be compared to that of mice treated with LNP-pDNA.

A subset of mice treated with LNP-construct or LNP-pDNA is given an additional IV injection boost of the corresponding LNPs at the same dose 1 month after the initial injection. Blood samples are collected at selected time points starting at 48 hours after the booster injection for up to 6 months. FVIII activity in blood will be analyzed by the chromogenic FVIII activity assay. FVIII expression profile in mice treated with LNP-construct will be compared to that of mice treated with corresponding LNP-pDNA.

### 6.17 Example 17 In Vitro Evaluation of FVIII Expression by Hairpin-Ended DNA Molecules in Cultured Hepatocytes

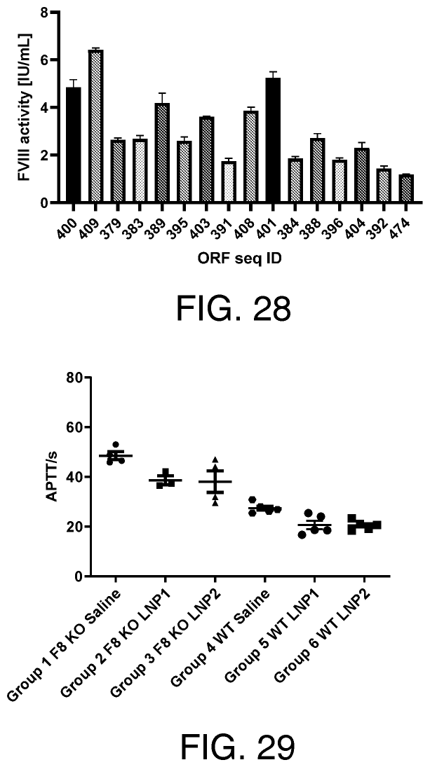

The nucleic acid sequences encoding the FVIII expression cassette were designed to encode for a modified left ITR (SEQ ID 417), a liver-specific promoter (SEQ ID 410), an enhancer (SEQ ID 416), FVIII gene variants (corresponding SEQ IDs are described in FIG. 28), a poly (a) signal and a modified right ITR (SEQ ID 418). Corresponding hairpin-ended DNA molecules were prepared and characterized by determining the level of FVIII activity after transfection into cultured hepatocytes. On the day prior to transfection, 20′000 HUH-7 cells were seeded in 100 μl in a 96 well plate. After 24 hours, the cells were transfected with 17.5 fmol of each construct of hairpinned DNA using Lipofectamine 3000 according to the manufacturer's manual. After 48 h of transfection, supernatant of cells was removed and transferred into a new 96-well plate and placed on ice (if tested later, supernatant was frozen at −80° C.). The harvested supernatants were tested for FVIII activity using the Chromogenix Coatest SP4 FVIII Activity assay. Absorbance values of test supernatants were interpolated from the standard curve provided by the kit and a calibration plasma.

### 6.18 Example 18 a Study to Evaluate Hairpin-Ended DNA LNP Formulations Via IV Delivery in Male B6.129S-F8^{tm1Smoc} and Wildtype C57BL/6 Mice

The following blood coagulation study was carried out to determine the activated partial thromboplastin (aPTT) clotting time after IV administration of LNP formulated hairpin-ended DNA encoding the FVIII variant protein sequence of SEQ ID 382 (for example, DNA SEQ ID NO: 385) under the regulation of a liver-specific promoter. Male wildtype C57BL/6 and FVIII knockout B6.129S-F8^{tm1Smoc} mice were divided into 3 groups each, of which two groups were IV administered with 1.5 mg/kg of hairpin-ended DNA molecules formulated in two LNP formulations each composing a different ionizable lipid species (for example, dlin-MC3-DMA and CKK-E12) and one group was treated with saline. After 3 days, the animals were sacrificed and blood samples were analyzed for aPTT using a Sysmex CS-2500 automated coagulation analyzer.

As shown in FIG. 29, it was observed that aPTT clotting time in mice in the studies was reduced in both knockout mice and wildtype mice as compared to control (saline) treated mice, suggesting that the LNP formulated FVIII encoding hairpin-ended DNA constructs disclosed herein could be effectively delivered and expressed to improve clotting time.

From the foregoing, it will be appreciated that, although specific embodiments have been described herein for the purpose of illustration, various modifications may be made without deviating from the spirit and scope of what is provided herein. All of the references referred to above are incorporated herein by reference in their entireties.

1. (canceled)

2. A method for treating a disease associated with reduced activity of Coagulation Factor VIII (FVIII) in a human patient, the method comprising administering to the patient a DNA molecule comprising an expression cassette comprising a transgene encoding human FVIII or a catalytically active fragment thereof, wherein the DNA molecule is contained within a single delivery vector, a biocompatible carrier (hybridosome) or a lipid nanoparticle.

3.-16. (canceled)

17. The method of claim 2, wherein the disease is Hemophilia A.

18. The method of claim 2, wherein the transgene comprises a sequence that is at least 90% identical to the sequence set forth in SEQ ID NO: 174, 175, 176, 177, 178, 179, 180, 181, 379, 380, 381, 383, 384, 385, 387, 386, 389, 391, 392, 393, 395, 396, 397, 399, 400, 401, 403, 404, 405, 407, 408, or 409.

19.-26. (canceled)

27. A double-stranded DNA molecule comprising in 5′ to 3′ direction of the top strand:
a. a first inverted repeat, wherein a first and a second restriction site for nicking endonuclease are arranged on opposite strands in proximity of the first inverted repeat such that nicking results in a top strand 5′ overhang or a bottom strand 3′ overhang comprising the first inverted repeat upon separation of the top from the bottom strand of the first inverted repeat;
b. an expression cassette comprising a transgene encoding human FVIII or a catalytically active fragment thereof; and
c. a second inverted repeat, wherein a third and a fourth restriction site for nicking endonuclease are arranged on opposite strands in proximity of the second inverted repeat such that nicking results in a top strand 3′ overhang or a bottom strand 5′ overhang comprising the second inverted repeat upon separation of the top from the bottom strand of the second inverted repeat.

28.-32. (canceled)

33. The DNA molecule of claim 27, wherein the first and the second inverted repeats are the same.

34. The DNA molecule of claim 27, wherein the first and/or the second inverted repeat is an inverted terminal repeat (ITR) of a parvovirus, wherein the parvovirus is a Dependoparvovirus, a Bocaparvovirus, an Erythroparvovirus, a Protoparvovirus, or a Tetraparvovirus.

35.-37. (canceled)

38. The DNA molecule of claim 34, wherein the ITR comprises a viral replication-associated protein binding sequence (“RABS”).

39.-40. (canceled)

41. The DNA molecule of claim 34, wherein the ITR does not comprise a replication-associated protein binding sequence (RABS).

42. (canceled)

43. The DNA molecule of claim 27, wherein the transgene comprises a sequence of SEQ ID NO: 174, 175, 176, 177, 178, 179, 180, 181, 379, 380, 381, 383, 384, 385, 387, 386, 389, 391, 392, 393, 395, 396, 397, 399, 400, 401, 403, 404, 405, 407, 408, or 409.

44.-49. (canceled)

50. The DNA molecule of claim 27, wherein the DNA molecule is a plasmid, wherein the plasmid comprises a bacterial origin of replication.

51.-53. (canceled)

54. The DNA molecule of claim 50, wherein the plasmid further comprises a fifth and a sixth restriction site for nicking endonuclease in the region 5′ to the first inverted repeat and 3′ to the second inverted repeat, wherein the fifth and sixth restriction sites for nicking endonuclease are:
a. on opposite strands; and
b. create a break in the double stranded DNA molecule such that the single strand overhangs of the break do not anneal at detectable levels inter- or intramolecularly under conditions that favor annealing of the first and/or second inverted repeat.

55.-56. (canceled)

57. The DNA molecule of claim 27, wherein the nicking endonuclease that recognizes the first, second, third, fourth, fifth, and/or sixth restriction site for nicking endonuclease is Nt. BsmAI; Nt. BtsCI; N. ALwl; N. BstNBI; N. BspD6I; Nb. Mval269I; Nb. BsrDI; Nt. BtsI; Nt. Bsal; Nt. BpulOI; Nt. BsmBI; Nb. BbvCI; Nt. BbvCI; or Nt. BspQI.

58. (canceled)

59. The DNA molecule of claim 27, wherein the nicking endonuclease that recognizes the first, second, third, fourth, fifth, and/or sixth restriction site for nicking endonuclease is a programmable nicking endonuclease.

60. (canceled)

61. The DNA molecule of claim 59, wherein the nicking endonuclease is a Cas nuclease.

62.-68. (canceled)

69. A composition comprising one or more DNA molecules of claim 27, and a pharmaceutically acceptable carrier, wherein the pharmaceutically acceptable carrier comprises a transfection agent, a nanoparticle, a lipid nanoparticle, a hybridosome, or a liposome.

70.-71. (canceled)

72. The use of a composition of claim 69 for preparing or manufacturing a medicament for ameliorating, preventing, delaying onset, or treating a disease or disorder associated with reduced activity of FVIII in a subject need thereof.

73. The DNA molecule of claim 27, wherein the DNA molecule:
a. lacks any functional replication-associated protein binding sequence (RABS); and/or
b. lacks any functional terminal resolution site (TRS); and/or
c. lacks or has reduced (by at least 50%, 60%, 70%, 80%, 90%, 95%, 98%, 99%, or at least 99.9%) PS AAV promoter activity; and/or
d. lacks any other elements required for viral replication.

74.-82. (canceled)
